(19) United States
(12) Patent Application Publication
(10) Pub. No.: US 2014/0073634 A1
(43) Pub. Date: Mar. 13, 2014

(54) HETEROCYCLIC MODULATORS OF HIF ACTIVITY FOR TREATMENT OF DISEASE

(71) Applicant: Institute For Applied Cancer Science/The University of Texas MD Anderson Cancer Center, Houston, TX (US)

(72) Inventors: Philip Jones, Houston, TX (US); Maria Emilia DiFrancesco, Houston, TX (US); Alessia Petrocchi, Houston, TX (US); Joe Marszalek, Houston, TX (US); Gang Liu, Houston, TX (US); Zhijun Kang, Houston, TX (US); Chris Carroll, Houston, TX (US); Timothy McAfoos, Houston, TX (US); Barbara Czako, Houston, TX (US)

(73) Assignee: Institute For Applied Cancer Science/The University of Texas MD Anderson Cancer Center, Houston, TX (US)

(21) Appl. No.: 13/974,266

(22) Filed: Aug. 23, 2013

Related U.S. Application Data
(60) Provisional application No. 61/743,132, filed on Aug. 24, 2012.

(51) Int. Cl. C07D 417/14; A61K 31/501; C07D 413/14; C07D 401/06; C07D 237/24; A61K 31/541; C07D 413/04; C07D 213/82; A61K 31/5377; A61K 31/4545; A61K 31/444; A61K 31/4439; A61K 45/06; A61K 31/496

(52) U.S. Cl. CPC C07D 417/14; A61K 45/06; A61K 31/501; C07D 413/14; C07D 401/06; C07D 237/24; A61K 31/496; C07D 413/04; C07D 213/82; A61K 31/5377; A61K 31/4545; A61K 31/444; A61K 31/4439; A61K 31/541
USPC 514/227.8; 514/252.02; 544/238; 544/364; 514/253.1; 514/253.12; 544/114; 514/236.5; 546/194; 514/318; 546/256; 514/333; 544/131; 514/236.2; 514/252.19; 544/333; 546/269.4; 514/340; 514/252.05; 544/58.2

(57) ABSTRACT

The present invention relates to compounds and methods which may be useful as inhibitors of HIF pathway activity for the treatment or prevention of cancer and other hypoxia-mediated diseases.

This application claims the benefit of priority of U.S. provisional application No. 61/743,132, filed Aug. 24, 2012, the disclosure of which is hereby incorporated by reference as if written herein in its entirety.

Disclosed herein are new heterocyclic compounds, compositions and their application as a pharmaceutical for the treatment of disease. Methods to inhibit HIF pathway activity through the degradation of the HIFα protein subunits in a human or animal subject are also provided for the treatment of diseases mediated by HIF pathway activity.

The heterodimeric HIF transcription factor is composed of a stable HIF1β (aka ARNT) and an oxygen regulatable HIFα subunit (HIF1α or EPAS1 (aka HIF2α)(Semenza, 2012b). Under normal physiological conditions, the capacity of the cell to degrade the HIFα subunits exceeds the amount of HIFα protein that is being synthesized. The HIFα subunit is regulated by hydroxylation at two key proline residues (ie. Pro⁴⁰² and Pro⁵⁶⁴ in HIF1α) by a family of proline hydroxylases (PHD1, PHD2 and PHD3), that utilize α-ketoglutarate and oxygen as substrates to generate hydroxylated HIFα, succinate and CO₂ (Kaelin and Ratcliffe, 2008). Hydroxylation of HIFα makes it a substrate for the VHL ubiquitin ligase complex, which promotes HIFα polyubiquitination, thus targeting HIFα for proteosomal degradation. This process is very rapid at normal oxygen levels, with a <5 minute half-life of HIFα protein, thus enabling rapid regulation of the complex and HIF activity in response to changes in oxygen levels (Maxwell et al., 1999).

Frequently in disease, the HIF pathway is activated by either reduced oxygen levels or genetic alterations that increase the amount of stabilized HIFα subunit (Semenza, 2012a). Increased HIFα levels occur through several mechanisms that include increased in HIFα subunit mRNA expression, HIFα protein translation, or through a decrease in HIFα protein degradation. Increased HIF leads to several biological pathways being activated through HIF mediated transcription of genes that promote stem cell maintenance, metabolic reprogramming, endothelial to mesenchymal transition (EMT), survival, proliferation, migration, pH regulation and angiogenesis.

A substantial body of preclinical experimentation and clinical evidence has implicated HIF as an important therapeutic target that is essential for the maintenance of a subset of tumors and a potential major contributor to therapeutic resistance and residual disease (Kaelin, 2011; Kaelin and Ratcliffe, 2008; Li et al., 2005; Semenza, 2012a; Semenza, 2012b). In numerous clinical studies, tumor hypoxia has been reported to correlate with poor prognosis and incomplete response to current therapeutic agents, including various chemotherapies as well as radiotherapy (Harada et al., 2012; Rohwer and Cramer, 2011; Wilson and Hay, 2011). This is most likely due to HIF regulation of procancerous mechanisms, including increased proliferation, activation of survival pathways such as autophagy, enhanced glycolysis as part of a metabolic reprogramming shift away from oxidative phosphorylation, increased migration/invasion promoting metastasis, maintenance of pluripotent “stem cell” population and stimulation of angiogenesis through the synthesis and secretion of pro-angiogenic growth factors.

The loss of any of several tumor suppressors (i.e. VHL, SDH, FH, TSC and others) and/or dysregulation of several oncogenic pathways (i.e. RAS and Pi3K) activate the HIF pathway and its downstream effector pathways, but do so in the presence of oxygen creating a “pseudohypoxic” state. These subsets of tumors become dependent on the HIF pathway for their continued growth. An example of a genetically driven HIF tumor indication is renal cell carcinoma (RCC), in which the tumor suppressor VHL is inactivated by mutation, deletion or promoter hypermethylation in 70% of tumors (Kim and Kaelin, 2004). VHL inactivation results in HIFα stabilization that is independent of oxygen concentration. In another example, tumors where either fumarate hydratase (FH) or a subunit in the succinate dehydrogenase (SDH) complex is inactivated, HIFα accumulation occurs due to inhibition of PHDs by succinate and fumarate (Bardella et al., 2011; Gill, 2012; Isaacs et al., 2005; Pollard et al., 2005). The lack of HIFα hydroxylation prevents VHL mediated degradation.

In other tumors, the Pi3K pathway is frequently mutated (ie., PTEN loss, AKT, PIK3CA, TSC1/2, LKB1 and others) ultimately leading to an increase in the activity of mammalian target of rapaycin (mTOR), which results in an increase in HIFα protein translation to the point where it overwhelms the degradation pathway. Therefore, in tumors with active Pi3K pathway, HIF pathway activity is frequently increased (Wouters and Koritzinsky, 2008). Taken together, in tumors where the HIF pathway is driven by specific genetic changes, therapeutic interventions that inactivate the HIF pathway in genetically driven HIF dependent tumors may provide substantial therapeutic benefit as monotherapy or as part of a combination therapy.

In addition to the activitation of HIF through genetic alterations, HIF is also activated in hypoxia that results from the tumor outgrowing the vasculature as well as a result of therapeutic intervention. HIF mediated survival of cells in hypoxia is a major contributor to resistance to therapies, lack of durable response and the foundation of residual disease. When tumor cells become hypoxic, several HIF dependent mechanisms prolong the survival of the cells in the harsh nutrient and oxygen deprived environment. These include genomic instability to promote adaptation (Klein and Glazer, 2010; Koi and Boland, 2011), metabolic reprogramming, induction of autophagy to recycle energy (Mazure and Pouyssegur, 2010), secretion of pro-angiogenic factors to promote neovascularization and cessation of pro-growth pathways. Severe hypoxia mediates innate resistance to radiotherapy and chemotherapy, which require oxygen and proliferation, respectively, as part of their mechanisms of action. Alternatively, resistance can be adaptive as in the case of anti-angiogenic therapies, such as anti-VEGF therapies, that create hypoxic niches due to the destruction of the vasculature, which creates more intratumoral hypoxia thus activating HIF and promoting its milieu of procancerous pathways. Multiple reports in a mouse models of cancer show that treatment with an anti-VEGF therapy promoted metastasis, most likely through HIF mediated activation of tumor cell migration/invasion (Ebos et al., 2009; Paez-Ribes et al., 2009). Hypoxia has also been proposed to promote genomic alteration by increasing DNA damage, including impairment of mismatch repair, nucleotide excision repair, double strand break repair and homologous recombination repair. The introduction of point mutations, frameshifts, insertions, deletions, amplifications and translocations give rise to tumor heterogeneity and evolution that provide the genetic alterations that enable adaptive resistance of tumors.

In most tumor types, inhibition of the HIF pathway activity will sensitize tumors to standard of care therapies such as anti-angiogenic therapies, radiotherapies, chemotherapies and targeted therapies by either improving the perfusion of drug and oxygen throughout the tumor via normalization of vascular function (Carmeliet and Jain, 2011; Chauhan et al., 2012) and by directly targeting the resistant HIF activated tumor cells to inhibit HIF mediated survival pathways.

In addition to cancer, inactivation of HIF pathway activate would be beneficial for conditions where activation of HIF promotes the disease state through aberrant survival or through promotion of neovascularization. These include traumatic shock, pulmonary arterial hypertension, obstructive sleep apnea, cardiovascular diseases such as cardiac arrhythmia and heart failure, diseases that involve neoangiogenesis such as ocular macular degeneration and rheumatoid arthritis, sepsis and inflammation and diseases of the lung and kidney where fibrosis occurs due HIF mediated EMT (Arjamaa et al., 2009; Semenza, 2012a; Westra et al., 2010).

To date, numerous small molecules have been reported that downregulate the HIF pathway via several direct and indirect mechanisms which target various HIF intervention points (Jones and Harris, 2012; Poon et al., 2009; Semenza, 2012b). These include reducing HIFα mRNA, reducing HIFα protein translation, reducing reactive oxygen species (ROS), increasing HIFα degradation, disrupting HIFα/HIF1β dimerization or the HIFα interaction with p300, a co-factor for HIF translation. Genetic and pharmacological inhibition of the HIF pathway utilizing RNAi, genetic ablation or via small molecule inhibitors have been reported to reduce the growth of tumors in preclinical models clearly establishing that the HIF pathway performs a critical function in tumor growth and maintenance (Onnis et al., 2009). Promoting HIFα degradation as part of a therapeutic intervention regime would be highly beneficial to patients. Herein we describe a series of selective small molecule inhibitors of HIF pathway activity that promote VHL and PHD mediated degradation of HIF.

Novel compounds and pharmaceutical compositions, certain of which have been found to inhibit HIF pathway activity have been discovered, together with methods of synthesizing and using the compounds including methods for the treatment of HIF pathway-mediated diseases in a patient by administering the compounds.

In certain embodiments of the present invention, compounds have structural Formula I:

(R₁)ₙ-A-Y₁—(B—(R₂)ₘ)-D-E-(R₃)ₚ (I)

or a salt thereof, wherein:

n is 0, 1, or 2;

p is 0, 1, or 2;

q is 0, 1, 2, 3, or 4;

u is 0, 1, or 2;

A is selected from the group consisting of aryl and heteroaryl;

B is selected from the group consisting of

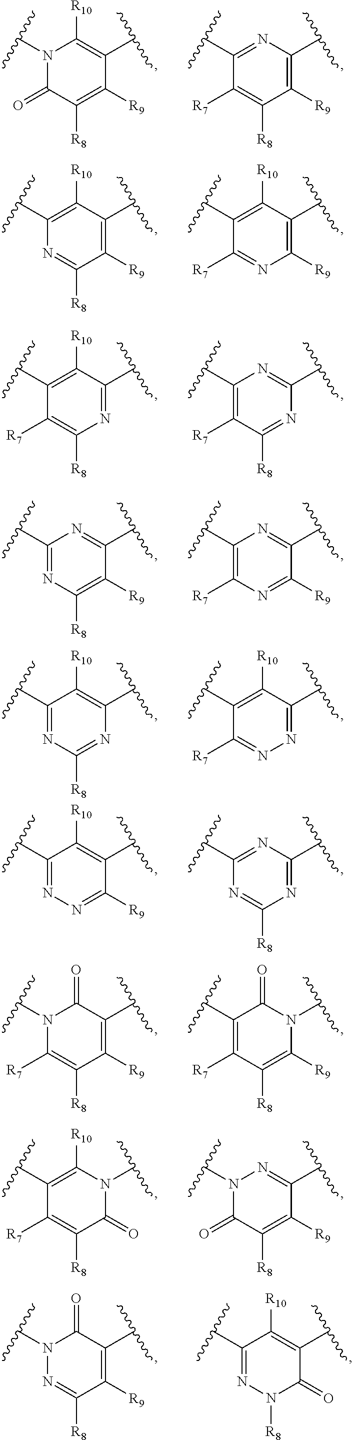

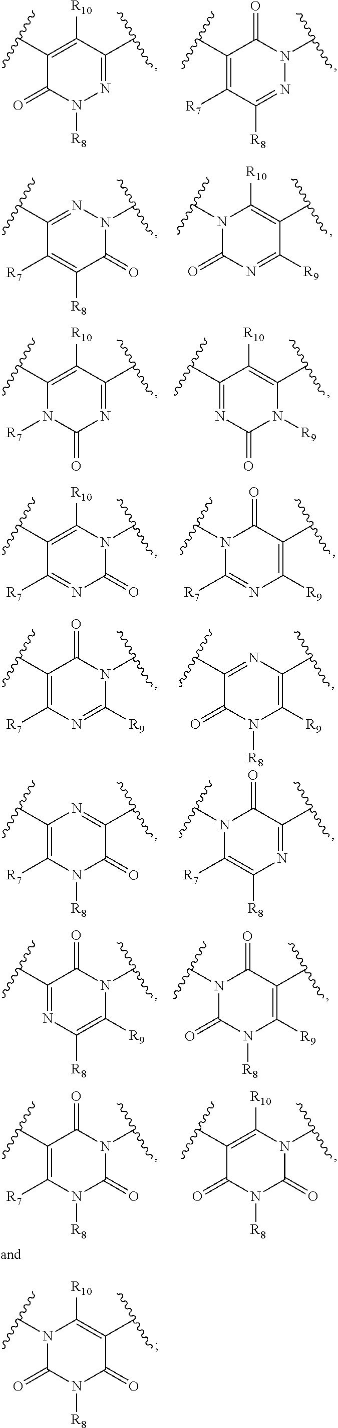

D is selected from the group consisting of alkyl, heteroalkyl, alkoxy, alkylthio, carbonyl, alkylcarbonyl, carboxyl, oxy, thio, sulfinyl, sulfonyl, sulfonamido, amino, amido, alkylamino, and heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, cyano, hydroxy, alkoxy, haloalkoxy, perhaloalkoxy, cycloalkyl, aryl, heterocycloalkyl, heteroaryl, and oxo, any of which may be optionally substituted;

E is selected from the group consisting of aryl and heteroaryl;

G is selected from the group consisting of saturated 3- to 7-membered cycloalkyl and saturated 3- to 7-membered heterocycloalkyl;

R₁ is selected from the group consisting of —Y₂-alkyl-N(R₄)R₅, hydrogen, deuterium, halogen, alkyl, alkenyl, alkynyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, mercaptyl, thiol, sulfonate, sulfonyl, sulfonamido, alkylsulfonyl, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy,

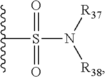

cycloalkylcarbonyl, arylcarbonyl, heterocycloalkylcarbonyl, heteroarylcarbonyl, cycloalkylalkyl, arylalkyl, heterocycloalkylalkyl, heterocycloalkylcarbonylalkyl, and heteroarylalkyl, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, amidoalkyl, acyl, carbonyl, carboxyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, alkoxyalkyl, carboxyl, cyano, hydroxy, alkoxy, haloalkoxy, perhaloalkoxy, oxo, alkylthio, thiol, acylthio, sulfonamido, alkylsulfonyl, amino, amido, carbamate, alkylamino, dialkylamino, alkylaminoalkyl, dialkylaminoalkyl, nitro, trisubstituted silyl, trisubstituted siloxy, cycloalkyl, aryl, heterocycloalkyl, heteroaryl, alkylheterocycloalkyl, any of which may be optionally substituted;

R₃ is selected from the group consisting of hydrogen, deuterium, halogen, alkyl, haloalkyl, perhaloalkyl, heteroalkyl, hydroxyalkyl, alkoxyalkyl, aminoalkyl, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, mercaptyl, thiol, haloalkylthio, perhaloalkylthio, cyanoalkylthio, haloalkylsulfonyl, alkylsulfonyl, alkoxyalkylsulfonyl, cyanoalkylsulfonyl, sulfonate, sulfonamido, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, trisubstituted silyl, —SF₅, —(C(R₃₁)(R₃₂))_{q}—O-alkyl, —(C(R₃₁)(R₃₂))_{q}—O-cycloalkyl, —S(O)ᵤ-alkyl, —S(O)ᵤ-cycloalkyl, cycloalkylthio, —CF₃, —OCF₃, —(C(R₃₁)(R₃₂))_{q}—OCF₃, saturated heterocycloalkyloxy, —(C(R₃₁)(R₃₂))_{q}—O-saturated heterocycloalkyl, —(C(R₃₁)(R₃₂))_{q}-saturated heterocycloalkyl, saturated heterocycloalkylthio, —S(O)ᵤ-saturated heterocycloalkyl, —(C(R₃₁)(R₃₂))_{q}—OCF₃,

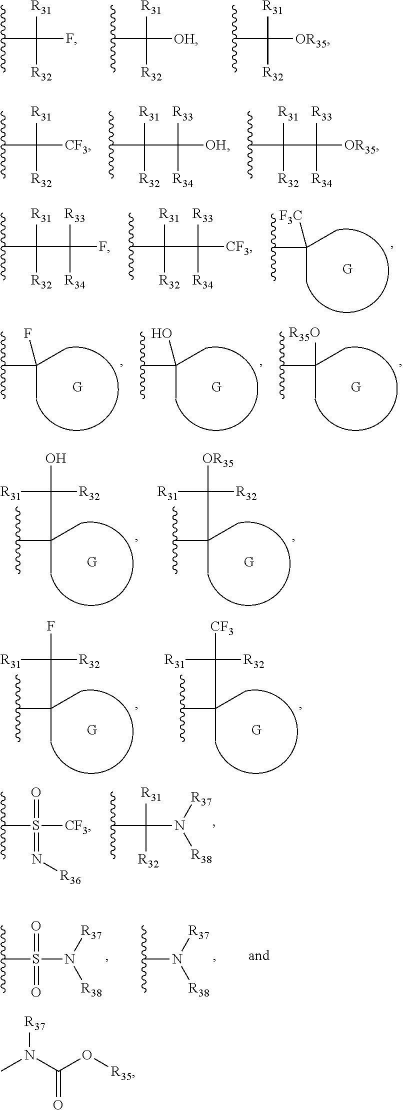

any of which may be optionally substituted;

R₄ and R₅ are independently selected from the group consisting of hydrogen, deuterium, alkyl, alkenyl, alkynyl, haloalkyl, perhaloalkyl, heteroalkyl, hydroxyalkyl, acyl, carbonyl, carboxyl, alkoxy, haloalkoxy, perhaloalkoxy, alkylsulfonyl, sulfonamido, amido, cycloalkyl, aryl, heterocycloalkyl, heteroaryl, cycloalkylalkyl, arylalkyl, heterocycloalkylalkyl, and heteroarylalkyl, or R₄ and R₅, taken together, form a heterocyloalkyl or heteroaryl, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonate, sulfonamido, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, any of which may be optionally substituted;

R₇, R₈, R₉, and R₁₀ are each independently selected from the group consisting of hydrogen, deuterium, halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, nitro, cycloalkyl, aryl, and heteroaryl, any of which may be optionally substituted;

R₃₁, R₃₂, R₃₃, R₃₄, and R₃₆ are independently selected from the group consisting of hydrogen, deuterium, alkyl, and perfluoroalkyl, any of which can be optionally substituted;

R₃₅ is selected from the group consisting of hydrogen, deuterium, alkyl, perfluoroalkyl, cycloalkyl, and saturated heterocycloalkyl, any of which can be optionally substituted;

R₃₇ and R₃₈ are independently selected from the group consisting of alkyl and perfluoroalkyl, or R₃₇ and R₃₈, taken together, form a heterocyloalkyl, any of which can be optionally substituted;

Y₁ is selected from the group consisting of alkyl, alkenyl, alkynyl, heteroalkyl, alkoxy, alkylthio, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, dialkylamino, and cycloalkyl, any of which may be optionally substituted; and

Y₂ is selected from the group consisting of a bond, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonate, sulfonamido, amino, amido, alkylamino, dialkylamino, carbamate, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, any of which may be optionally substituted.

Certain compounds disclosed herein may possess useful HIF pathway inhibiting activity, and may be used in the treatment or prophylaxis of a disease or condition in which the HIF pathway plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting the HIF pathway. Other embodiments provide methods for treating a HIF pathway-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of the HIF pathway.

In further embodiments,

if A is phenyl, Y₁ is —CH₂—, B is

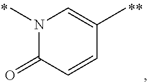

### D is

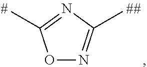

E is phenyl, n is 1, p is 1, and R₃ is —OCF₃, then R₁ is not chloro, bromo, methyl, —NH₂, —NO₂, —C(═O)Cl, —CO₂H,

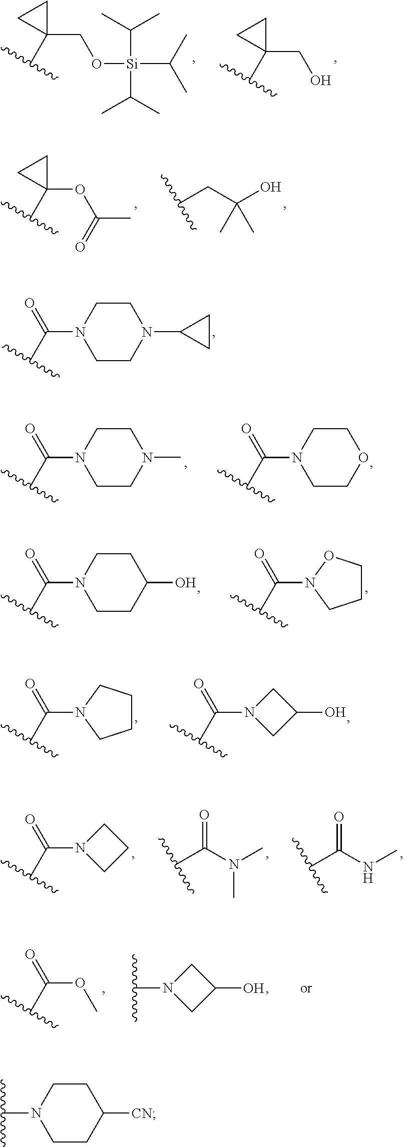

if A is pyridyl, Y₁ is —CH₂—, B is

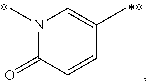

### D is

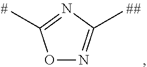

E is phenyl, n is 1, p is 1, and R₃ is —OCF₃, then R₁ is not chloro, bromo,

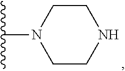 [SMILES: *N1CCNCC1]

### —NHCH₃, —NHCH₂CH₃,

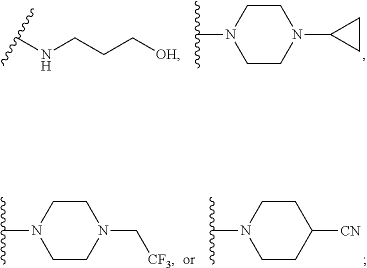

if A is phenyl, Y₁ is —CH₂—, B is

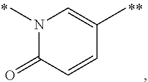

### D is

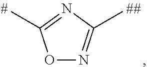

E is phenyl, n is 1, p is 1, and R₃ is —SCF₃, then R₁ is not

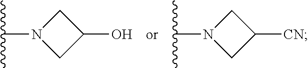 [SMILES: *N1CC(O)C1 ; "or" ; *N1CC(C#N)C1]

if A is pyridyl Y₁ is —CH₂—, B is

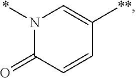

### D is

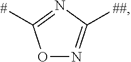

E is phenyl, n is 1, p is 1, and R₃ is —SCF₃, then R₁ is not chloro,

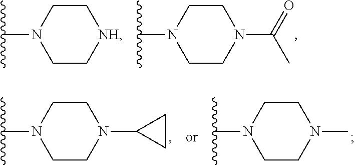

if A is phenyl, Y₁ is —CH₂—, B is

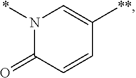

### D is

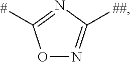

E is phenyl, n is 1, p is 1, and R₃ is —C(CH₃)₂CF₃, then R₁ is not chloro, bromo, methyl, —NH₂, —NO₂, —C(═O)Cl, —CO₂H,

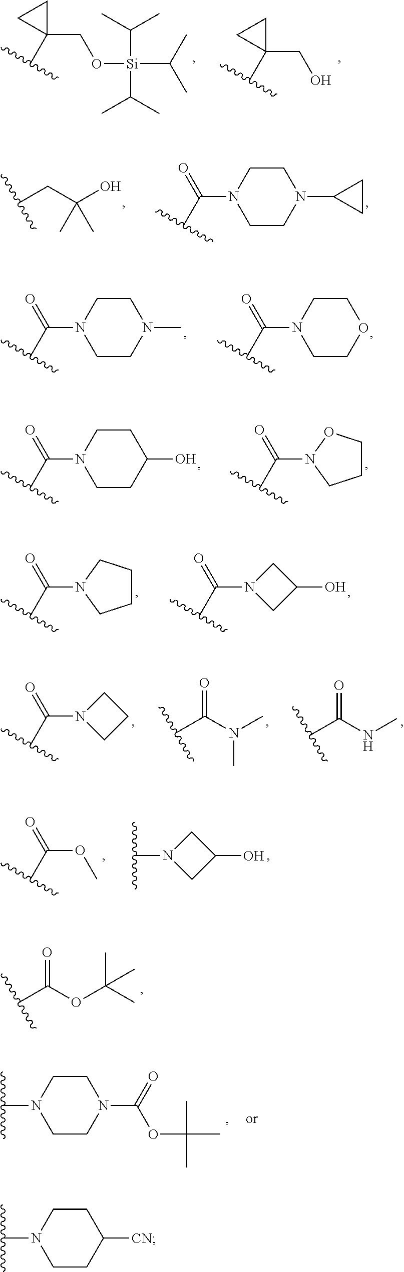

if A is pyridyl, Y₁ is —CH₂—, B is

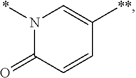

### D is

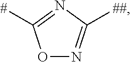

E is phenyl, n is 1, p is 1, and R₃ is —C(CH₃)₂CF₃, then R₁ is not chloro, —NHCH₃, —NHCH₂CH₃,

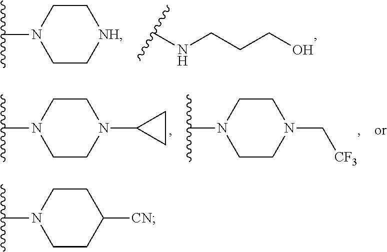

if A is phenyl, Y₁ is —CH₂—, B is

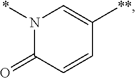

### D is

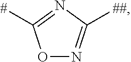

E is phenyl, n is 1, p is 1, and R₃ is

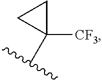 [SMILES: *C1(C(F)(F)F)CC1]

then R₁ is not bromo,

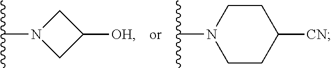 [SMILES: *N1CC(O)C1 ; "or" ; *N1CCC(C#N)CC1]

if A is pyridyl, Y₁ is —CH₂—, B is

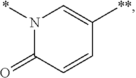

### D is

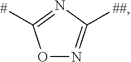

E is phenyl, n is 1, p is 1, and R₃ is

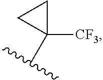 [SMILES: *C1(C(F)(F)F)CC1]

then R₁ is not chloro,

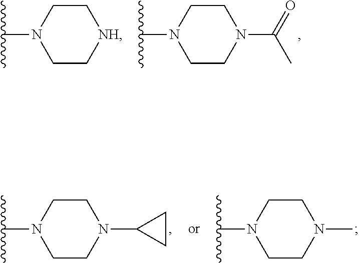

if A is phenyl, Y₁ is —CH₂—, B is

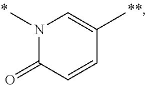

### D is

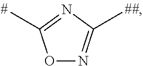

E is phenyl, n is 1, p is 1, and R₃ is methyl, then R₁ is not chloro;

if A is phenyl, Y₁ is —CH₂—, B is

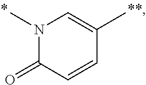

### D is

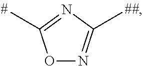

E is phenyl, n is 1, p is 1, and R₃ is chloro, then R₁ is not methyl;

if A is phenyl, Y₁ is —CH₂—, B is

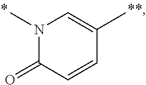

### D is

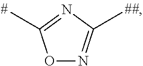

E is phenyl, n is 1, p is 1, and R₃ is methoxy, then R₁ is not methyl;

if A is phenyl, Y₁ is —CH₂—, B is

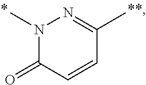

### D is

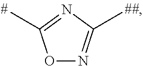

E is phenyl, n is 1, p is 1, and R₃ is —C(CH₃)₂CF₃, then R₁ is not —C(═O)Cl, —CO₂H,

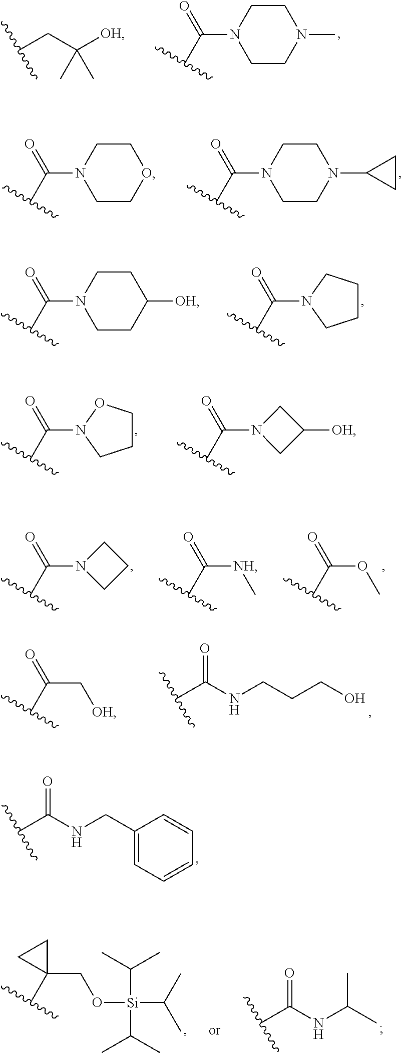

if A is phenyl, Y₁ is —CH₂—, B is

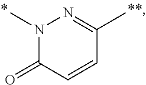

### D is

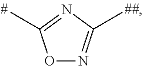

E is phenyl, n is 1, p is 1, and R₃ is —OCF₃, then R₁ is not methyl, —C(═O)Cl, —CO₂H, bromine,

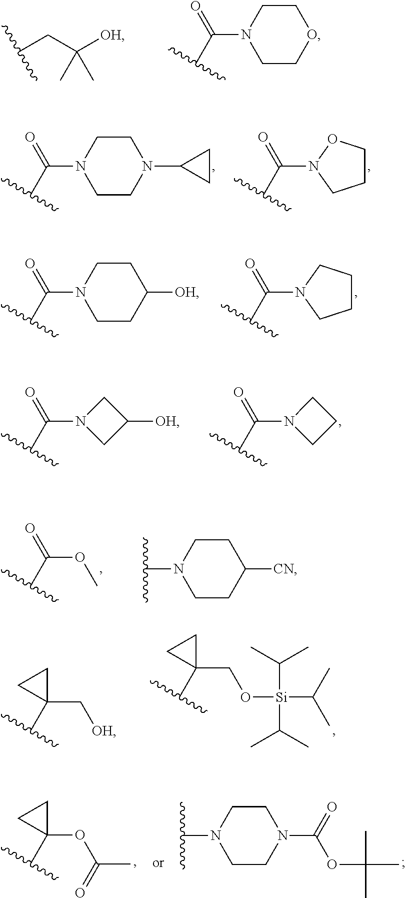

if A is phenyl, Y₁ is —CH₂—, B is

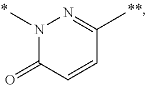

### D is

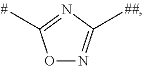

E is phenyl, n is 1, p is 1, and R₃ is methyl, then R₁ is not methoxy;

if A is pyridyl, Y₁ is —CH₂—, B is

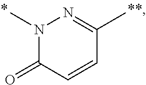

### D is

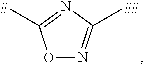

E is phenyl, n is 1, p is 1, and R₃ is —C(CH₃)₂CF₃, then R₁ is not chloro, —NHCH₃,

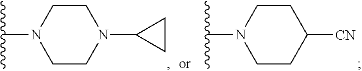

if A is pyridyl, Y₁ is —CH₂—, B is

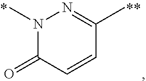

### D is

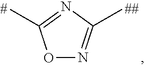

E is phenyl, n is 1, p is 1, and R₃ is —OCF₃, then R₁ is not chloro, —NHCH₃,

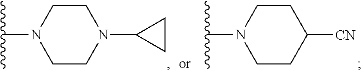

and

wherein * represents the point of attachment to Y₁ and ** represents the point of attachment to D, and # represents the point of attachment to B and ## represents the point of attachment to E.

In further embodiments, D is selected from the group consisting of

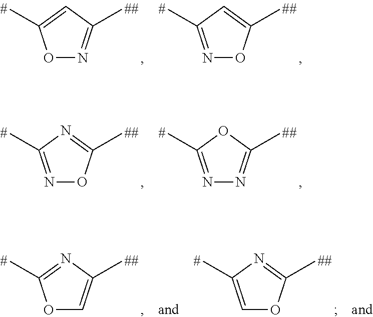

# represents the point of attachment to B and ## represents the point of attachment to E.

In further embodiments, if A is

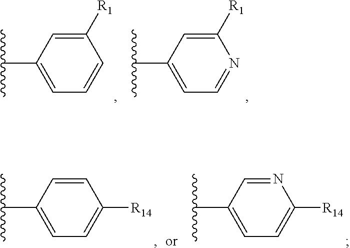

Y₁ is —CH₂—;

B is

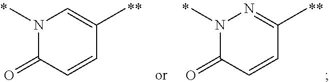

D is

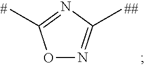

E is

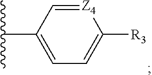

Z₄ is N or CR₁₇;

R₃ is halogen, cyano, —SF₅, tri-C₁-C₄ alkylsilyl, C₁-C₆ alkyl, C₁-C₆ alkoxy, C₁-C₆ alkylthio, C₁-C₆ alkylsulfonyl, C₃-C₆ cycloalkyl, or 4- to 6-membered hererocycloalkyl, wherein said C₁-C₆ alkyl, C₁-C₆ alkoxy, C₁-C₆ alkylthio, and C₁-C₆ alkylsulfonyl are optionally substituted with hydroxy, methoxy, ethoxy, and one to six fluorine atoms, and wherein said C₃-C₆ cycloalkyl and 4- to 6-membered hererocycloalkyl are optionally substituted with one to two substituents selected from the group consisting of fluoro, C₁-C₄ alkyl, trifluoromethyl, hydroxy, methoxy, and ethoxy;

R₁₄ is chloro, cyano, nitro, amino, C₁-C₄ alkyl, C₁-C₄ alkoxy, or C₁-C₄ monoalkylamino, wherein said C₁-C₄ alkyl, C₁-C₄ alkoxy, and C₁-C₄ monoalkylamino are optionally substituted by hydroxyl or one to three fluorine atoms;

R₁₇ is hydrogen, fluoro, chloro, methyl, or trifluoromethyl;

R₂₀₁ and R₂₀₂, taken together with the nitrogen atom to which they are attached, form a 4- to 6-membered heterocycloalkyl which can contain a further heteroatom selected from the group consisting of NR₂₀₅, O, S, and S(O)₂, and which is optionally substituted by one to two substituents selected from the group consisting of fluoro, cyano, C₁-C₄ alkyl, hydroxy, methoxy, and ethoxy, wherein said C₁-C₄ alkyl is optionally substituted with hydroxy and one to three fluorine atoms;

R₂₀₃ and R₂₀₄, is hydrogen or C₁-C₄ alkyl, wherein said C₁-C₄ alkyl is optionally substituted with hydroxy, methoxy, ethoxy, phenyl, and one to three fluorine atoms; or R₂₀₃ and R₂₀₄, taken together with the nitrogen atom to which they are attached, form a 4- to 6-membered heterocycloalkyl which can contain a further heteroatom selected from the group consisting of NR₂₀₅, O, S, and S(O)₂, and which is optionally substituted by one to two substituents selected from the group consisting of fluoro, cyano, C₁-C₄ alkyl, hydroxy, methoxy, and ethoxy, wherein said C₁-C₄ alkyl is optionally substituted with hydroxy and one to three fluorine atoms; and

R₂₀₅ is C₁-C₄ alkyl, C₃-C₆ cycloalkyl, C₁-C₄ alkylcarbonyl, or C₁-C₄ alkoxycarbonyl, wherein said C₁-C₄ alkyl is optionally substituted with one to three fluorine atoms;

then R₁ is not C₁-C₆ alkyl, C₃-C₆ cycloalkyl, C₁-C₄ alkoxycarbonyl, —NR₂₀₁R₂₀₂, or —C(═O)—NR₂₀₃R₂₀₄, wherein said C₁-C₆ alkyl is optionally substituted with hydroxy and one to three fluorine atoms, and said C₃-C₆ cycloalkyl is optionally substituted with a substituent selected from the group consisting hydroxy, C₁-C₄ hydroxyalkyl, and C₁-C₄ alkoxycarbonyl.

In further embodiments, B is selected from the group consisting of

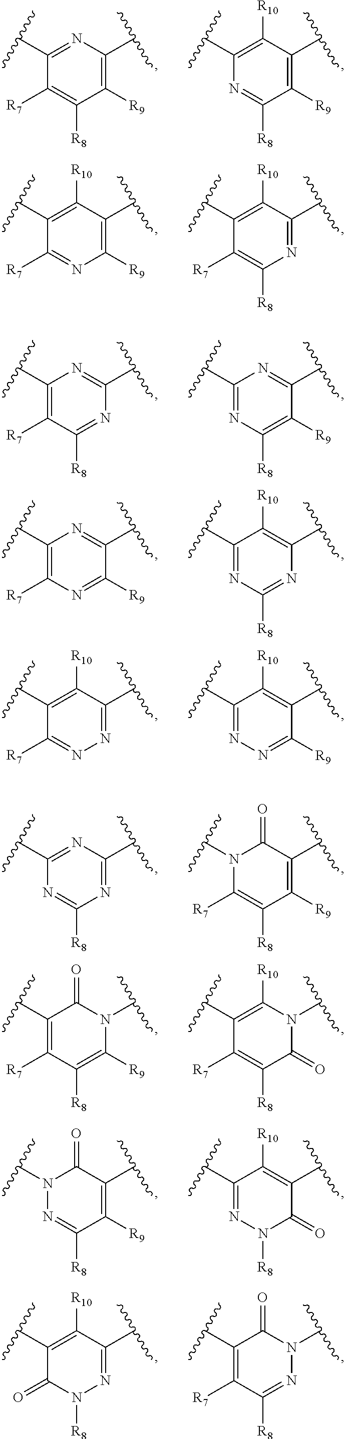

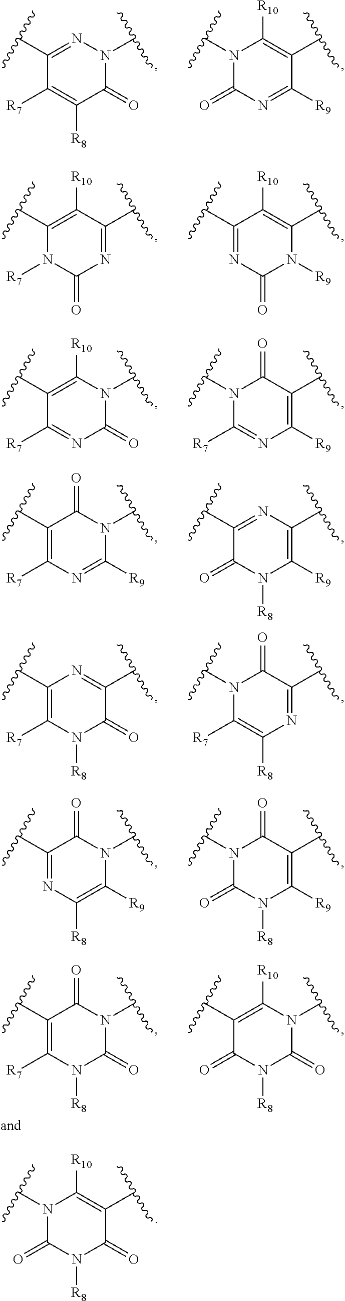

In further embodiments, D is selected from the group consisting of alkyl, heteroalkyl, alkoxy, alkylthio, carbonyl, alkylcarbonyl, carboxyl, oxy, thio, sulfinyl, sulfonyl, sulfonamido, amino, amido, alkylamino, and 6-membered heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, cyano, hydroxy, alkoxy, haloalkoxy, perhaloalkoxy, cycloalkyl, aryl, heterocycloalkyl, heteroaryl, and oxo, any of which may be optionally substituted.

In further embodiments, E is selected from the group consisting of bicyclic aryl, bicyclic heteroaryl, and 5-membered heteroaryl.

In further embodiments, R₃ is selected from the group consisting of hydrogen, deuterium, aminoalkyl, acyl, carbonyl, carboxyl, cyanoalkyl, hydroxyoxo, mercaptyl, thiol, cyanoalkylthio, alkoxyalkylsulfonyl, cyanoalkylsulfonyl, sulfonate, sulfonamido, amino, amido, alkylamino, dialkylamino, carbamate, nitro, arylcycloalkyloxy, aryloxy, heterocycloalkyloxy, heteroaryloxy, cycloalkylalkyl, arylalkyl, heterocycloalkylalkyl, and heteroarylalkyl, —(C(R₃₁)(R₃₂))_{q}—O-alkyl, —(C(R₃₁)(R₃₂))_{q}-β-cycloalkyl, —S(O)ᵤ-cycloalkyl, cycloalkylthio, —(C(R₃₁)(R₃₂))_{q}—OCF₃, saturated heterocycloalkyloxy, —(C(R₃₁)(R₃₂))_{q}—O-saturated heterocycloalkyl, —(C(R₃₁)(R₃₂))_{q}— saturated heterocycloalkyl, saturated heterocycloalkylthio, —S(O)ᵤ-saturated heterocycloalkyl, —(C(R₃₁)(R₃₂))_{q}—OCF₃,

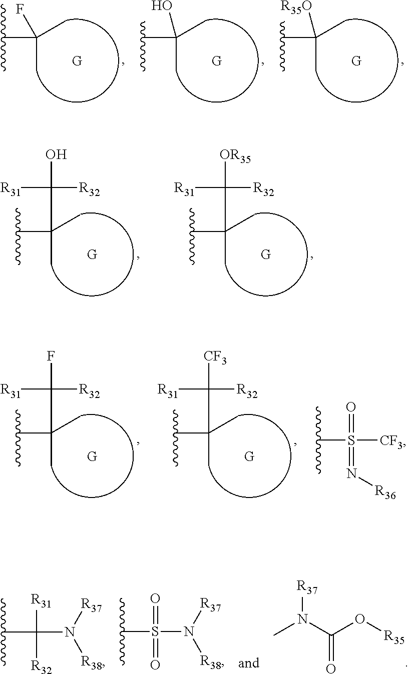

In further embodiments, R₁ is selected from the group consisting of —Y₂-alkyl-N(R₄)R₅, hydrogen, deuterium, alkenyl, alkynyl, aminoalkyl, acyl, carboxylalkyl, cyano, hydroxy, alkoxy, haloalkoxy, perhaloalkoxy, oxo, alkylthio, thiolalkyl, mercaptyl, thiol, sulfonate, sulfonyl, sulfonamido, alkylsulfonyl, carbamate, aryl, heteroaryl, cycloalkyloxy, aryloxy, heterocycloalkyloxy, heteroaryloxy,

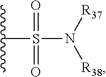

cycloalkylcarbonyl, arylcarbonyl, heteroarylcarbonyl, cycloalkylalkyl, arylalkyl, heterocycloalkylalkyl, heterocycloalkylcarbonylalkyl, and heteroarylalkyl, any of which can be optionally substituted with one or more substituents selected from the group consisting of hydrogen, deuterium, alkyl, alkenyl, alkynyl, haloalkyl, perhaloalkyl, heteroalkyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, alkoxyalkyl, carboxyl, cyano, hydroxy, haloalkoxy, perhaloalkoxy, oxo, alkylthio, thiol, acylthio, sulfonamido, alkylsulfonyl, amino, amido, carbamate, alkylamino, dialkylamino, alkylaminoalkyl, dialkylaminoalkyl, nitro, trisubstituted silyl, trisubstituted siloxy, cycloalkyl, aryl, heterocycloalkyl, heteroaryl, alkylheterocycloalkyl, any of which may be optionally substituted.

In further embodiments, at least one of R₇, R₈, R₉, and R₁₀ is halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, nitro, cycloalkyl, aryl, and heteroaryl, any of which may be optionally substituted.

In further embodiments,

Y₁ is selected from the group consisting of alkyl, alkenyl, alkynyl, heteroalkyl, alkoxy, alkylthio, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, dialkylamino, and cycloalkyl, any of which may be optionally substituted; and

if Y₁ is C₁ alkyl, it is substituted with at least one substituent selected from the group consisting of halogen, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, dialkylamino, and cycloalkyl, any of which may be optionally substituted.

In further embodiments,

A is selected from the group consisting of aryl and mono- or bicyclic heteroaryl;

B is selected from the group consisting of

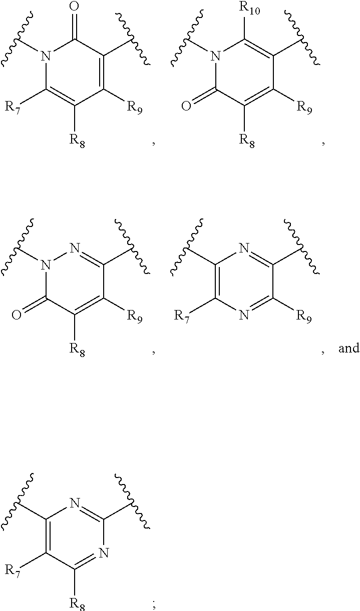

D is selected from the group consisting of amido, 5-membered heteroaryl, and 6-membered heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, cyano, hydroxy, alkoxy, haloalkoxy, perhaloalkoxy, cycloalkyl, aryl, heterocycloalkyl, heteroaryl, and oxo, any of which may be optionally substituted;

E is selected from the group consisting of phenyl, 5-membered heteroaryl, 6-membered heteroaryl, and 9-membered bicyclic heteroaryl;

R₄ and R₅ are independently selected from the group consisting of hydrogen, deuterium, alkyl, alkenyl, alkynyl, haloalkyl, perhaloalkyl, heteroalkyl, hydroxyalkyl, acyl, carbonyl, carboxyl, alkoxy, haloalkoxy, perhaloalkoxy, alkylsulfonyl, sulfonamido, amido, cycloalkyl, aryl, heterocycloalkyl, heteroaryl, cycloalkylalkyl, arylalkyl, heterocycloalkylalkyl, and heteroarylalkyl, or R₄ and R₅, taken together, form a heterocyloalkyl or heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonamido, amino, amido, alkylamino, dialkylamino, carbamate, and cycloalkyl, any of which may be optionally substituted;

R₇, R₈, R₉, and R₁₀ are each independently selected from the group consisting of null, hydrogen, deuterium, halogen, alkyl, haloalkyl, perhaloalkyl, heteroalkyl, hydroxyalkyl, cyano, hydroxy, alkoxy, haloalkoxy, perhaloalkoxy, alkylthio, amino, alkylamino, cycloalkyl, aryl, and heteroaryl;

Y₁ is alkyl, which can be optionally substituted with one or more substituents selected from the group consisting of hydrogen, deuterium, alkyl, cycloalkyl, and halogen; and

Y₂ is selected from the group consisting of a bond, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonamido, amino, amido, alkylamino, dialkylamino, carbamate, and cycloalkyl, any of which may be optionally substituted.

In further embodiments,

D is selected from the group consisting of —C(═O)NR₁₁—, 5-membered heteroaryl, and 6-membered heteroaryl;

E is selected from the group consisting of phenyl, pyrimidine, 1,3-benzodioxol, indole, and 1-benzofuran;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy,

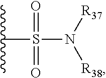

cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;

R₃ is selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted;

R₁₁ is selected from the group consisting of hydrogen, deuterium, alkyl, haloalkyl, perhaloalkyl, heteroalkyl, hydroxyalkyl, cycloalkyl, aryl, heterocycloalkyl, and heteroaryl, any of which may be optionally substituted;

Y₁ is —CH₂—; and

Y₂ is selected from the group consisting of a bond, carbonyl, amino, and alkylamino.

In further embodiments,

A is selected from the group consisting of phenyl, 5-membered heteroaryl, and 6-membered heteroaryl;

E is phenyl;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, acyl, carboxylalkyl, carboxyl, carbonyl, cyano, hydroxy, alkoxy, haloalkoxy, perhaloalkoxy, oxo, thiolalkyl, sulfonyl, sulfonamido, alkylsulfonyl, amino, amido, alkylamino, dialkylamino, nitro, heterocycloalkyl, heterocycloalkyloxy,

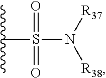

heterocycloalkylcarbonylalkyl, and heterocycloalkylcarbonyl, any of which can be optionally substituted with one or more substituents selected from the group consisting of hydrogen, deuterium, halogen, alkyl, alkenyl, amidoalkyl, acyl, carboxylalkyl, hydroxyalkylcarbonyl, alkynylcarbonyl, heteroalkyl, hydroxyalkyl, alkoxyalkyl, carboxyl, cyano, hydroxy, alkoxy, oxo, sulfonamido, alkylsulfonyl, amino, amido, carbamate, dialkylamino, dialkylaminoalkyl, trisubstituted siloxy, cycloalkyl, heterocycloalkyl, alkylheterocycloalkyl, any of which may be optionally substituted;

R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, and 4- to 6-membered heterocycloalkyl; and

R₁₁ is selected from the group consisting of hydrogen, deuterium, alkyl, and cycloalkyl, any of which may be optionally substituted.

In further embodiments,

n is 1;

p is 1; and

R₇, R₈, R₉, and R₁₀ are each independently selected from the group consisting of alkyl, haloalkyl, perhaloalkyl, hydroxy, and cyclopropyl.

In certain embodiments, disclosed herein are compounds having structural Formula II

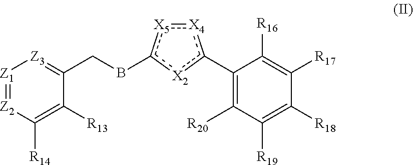

or a salt thereof, wherein:

B is selected from the group consisting of

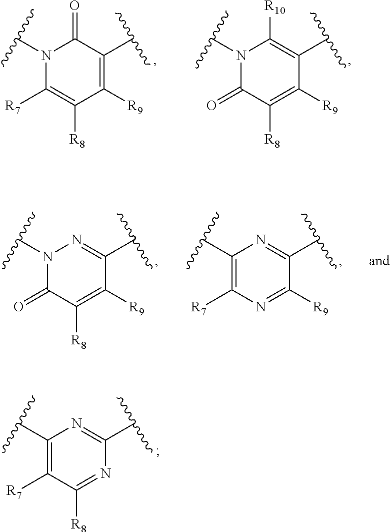

X₂, X₄, and X₅ are independently selected from the group consisting of CR₂₁, N, O, and S, and wherein, X₂, X₄, and X₅, taken together, form a 5-membered heteroaryl;

Z₁ and Z₂ are independently selected from the group consisting of N, NR₁, C═O, and CR₁;

Z₃ is selected from the group consisting of N, NR₁₂, C═O, and CR₁₂;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy,

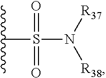

cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;

R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;

R₁₂, R₁₃, and R₁₄ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted;

R₁₆, R₁₉, and R₂₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and cycloalkyl, any of which may be optionally substituted;

R₁₇ and R₁₈ are independently selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted; and

R₂₁ is selected from the group consisting of null, hydrogen, deuterium, halogen, alkyl, haloalkyl, perhaloalkyl, heteroalkyl, hydroxyalkyl, cyano, hydroxy, alkoxy, haloalkoxy, perhaloalkoxy, alkylthio, amino, alkylamino, and dialkylamino.

In further embodiments,

two of X₂, X₄, and X₅ are N, and one of X₂, X₄, and X₅ are O; or

one of X₂, X₄, and X₅ is N; one of X₂, X₄, and X₅ is O; and one of X₂, X₄, and X₅ is CH.

In further embodiments,

at least one of Z₁ or Z₂ is CR₁;

Z₃ is CR₁₂;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, acyl, carboxylalkyl, carboxyl, carbonyl, cyano, hydroxy, alkoxy, haloalkoxy, perhaloalkoxy, oxo, thiolalkyl, sulfonyl, sulfonamido, alkylsulfonyl, amino, amido, alkylamino, dialkylamino, nitro, heterocycloalkyl, heterocycloalkyloxy,

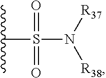

heterocycloalkylcarbonylalkyl, and heterocycloalkylcarbonyl, any of which can be optionally substituted with one or more substituents selected from the group consisting of hydrogen, deuterium, halogen, alkyl, alkenyl, amidoalkyl, acyl, carboxylalkyl, hydroxyalkylcarbonyl, alkynylcarbonyl, heteroalkyl, hydroxyalkyl, alkoxyalkyl, carboxyl, cyano, hydroxy, alkoxy, oxo, sulfonamido, alkylsulfonyl, amino, amido, carbamate, dialkylamino, dialkylaminoalkyl, trisubstituted siloxy, cycloalkyl, heterocycloalkyl, alkylheterocycloalkyl, any of which may be optionally substituted;

R₁₂, R₁₃, and R₁₄ are hydrogen;

R₁₆, R₁₇, R₁₉, and R₂₀ are hydrogen; and

R₂₁ is selected from the group consisting of null, hydrogen, deuterium, halogen, and alkyl.

In further embodiments,

R₁ is selected from the group consisting of hydrogen, deuterium, fluorine, bromine, cyano, methyl, isopropyl,

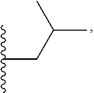 [SMILES: *CC(C)C]

ethylene,

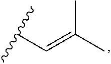 [SMILES: *C=C(C)C]

trifluoromethyl, bromomethyl, hydroxymethyl, difluoromethoxy, methoxy, ethoxy, isopropoxy, hydroxy, nitro, acetyl, carboxyl, —CO₂CH₃,

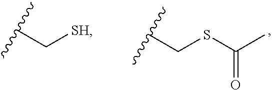

—SO₂CH₃, —SO₂CH₂CH₃, SO₂CH₂CH₂CH₃, —SO₂NH₂,

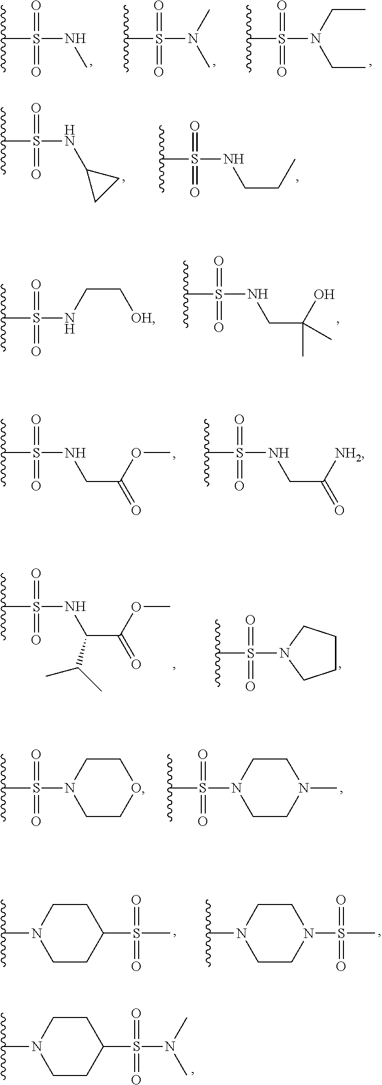

amino, methylamino, dimethylamino,

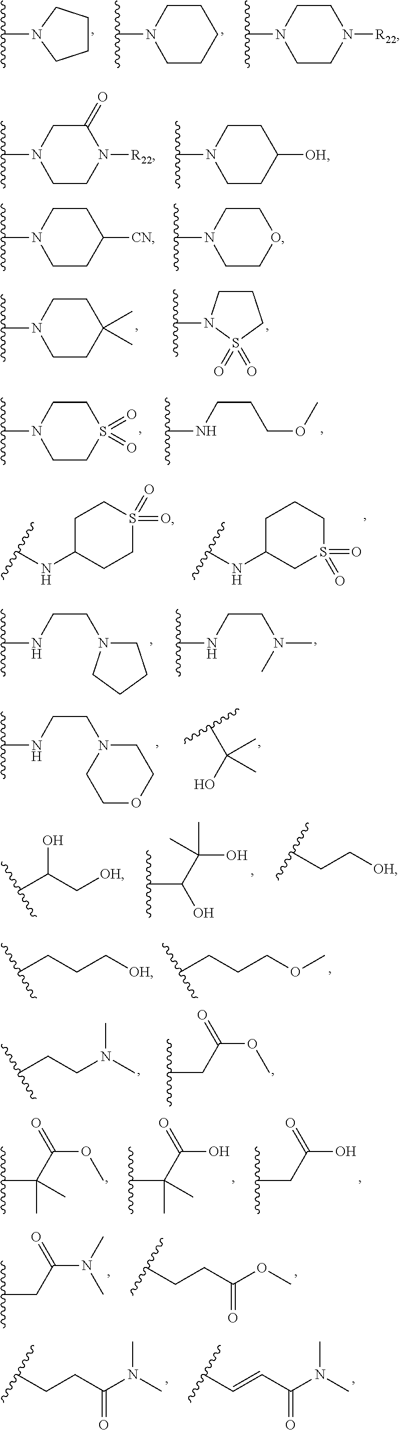

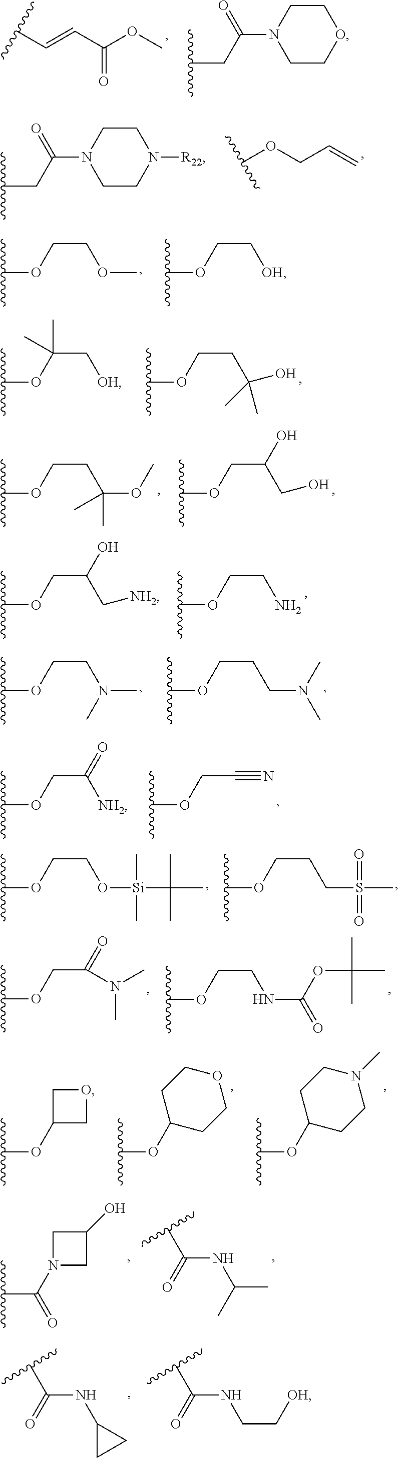

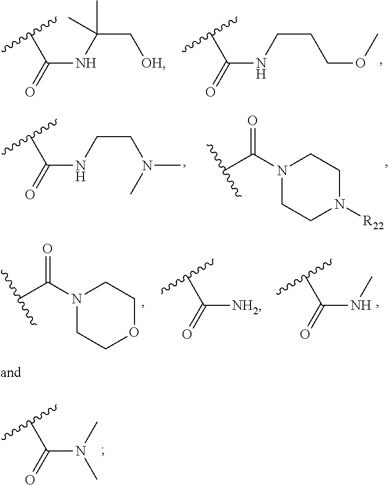

R₇, R₈, R₉, and R₁₀ are each independently selected from the group consisting of hydrogen, deuterium, C₁-C₃ alkyl, 4-pyridyl, and cyclopropyl;

R₁₈ is selected from the group consisting of hydrogen, deuterium, halogen, methyl, isopropyl, tert-butyl, cyclopropyl, cyclohexyl, acetyl, hydroxymethyl, methoxymethyl, methoxy, isopropoxy, methylamino, dimethylamino, methylthio, cyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃,

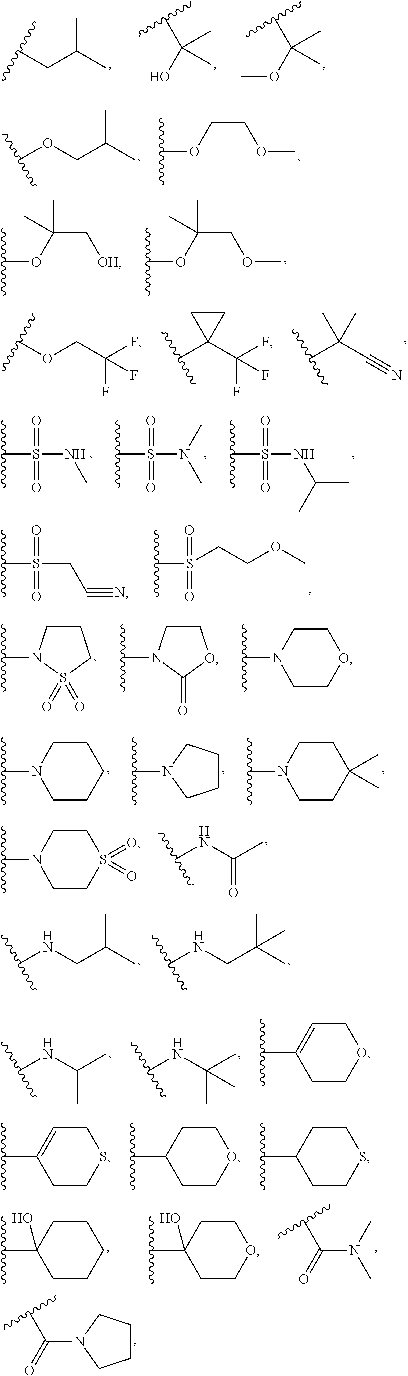

trifluoromethyl, trifluoromethylthio, difluoromethoxy, and trifluoromethoxy; and

R₂₂ is selected from the group consisting of hydrogen, deuterium, methyl, acetyl,

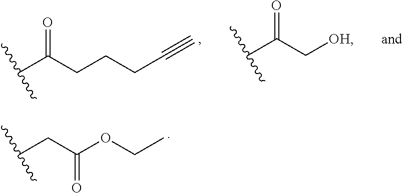

In further embodiments,

R₁ is selected from the group consisting of hydrogen, deuterium, chloro, cyano, methyl, ethylene,

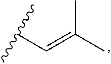 [SMILES: *C=C(C)C]

bromomethyl, hydroxymethyl, difluoromethoxy, methoxy, ethoxy, hydroxy, nitro, —CO₂CH₃,

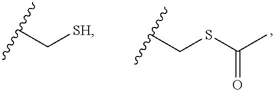

—SO₂CH₃, —SO₂CH₂CH₃, SO₂CH₂CH₂CH₃, —SO₂NH₂,

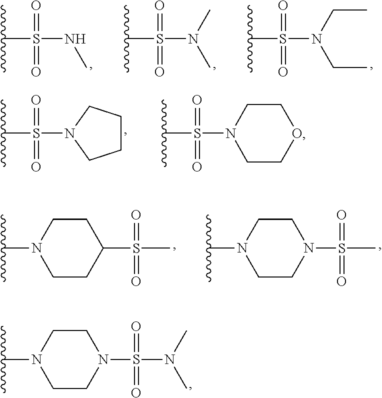

dimethylamino,

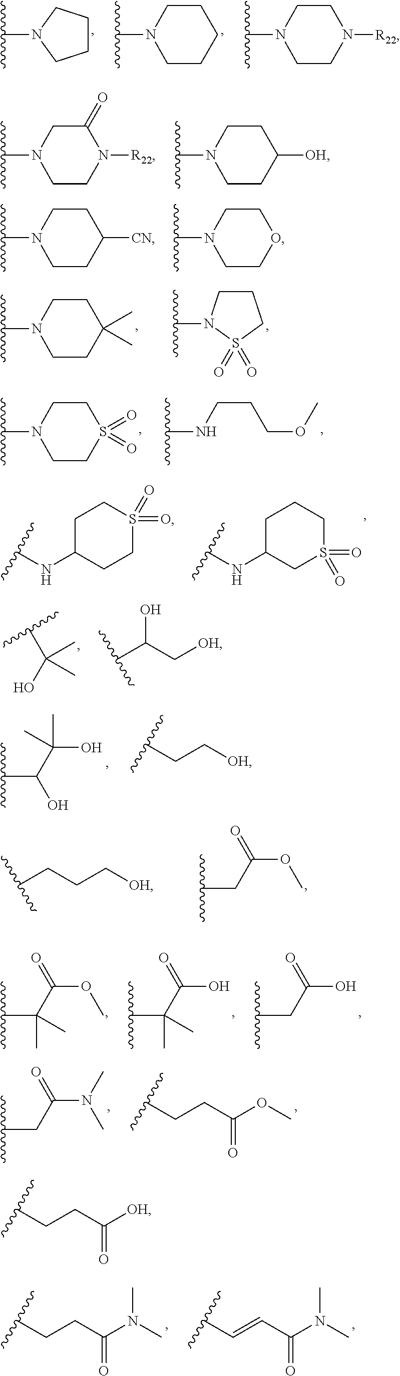

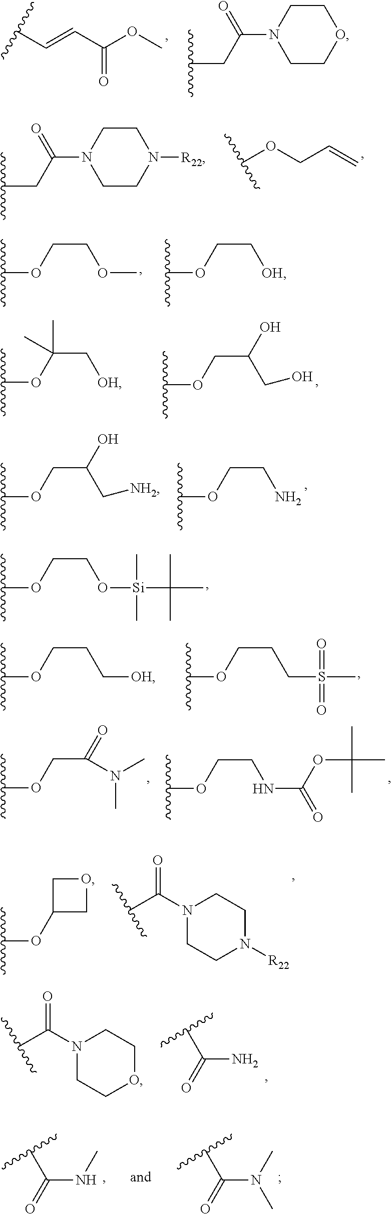

R₇, R₈, R₉, and R₁₀ are each independently selected from the group consisting of hydrogen, deuterium and methyl;

R₁₈ is selected from the group consisting of hydrogen, deuterium, chloro, methyl, isopropyl, tert-butyl, cyclopropyl, cyclohexyl, acetyl, hydroxymethyl, methoxymethyl, methoxy, isopropoxy, methylaminocyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃,

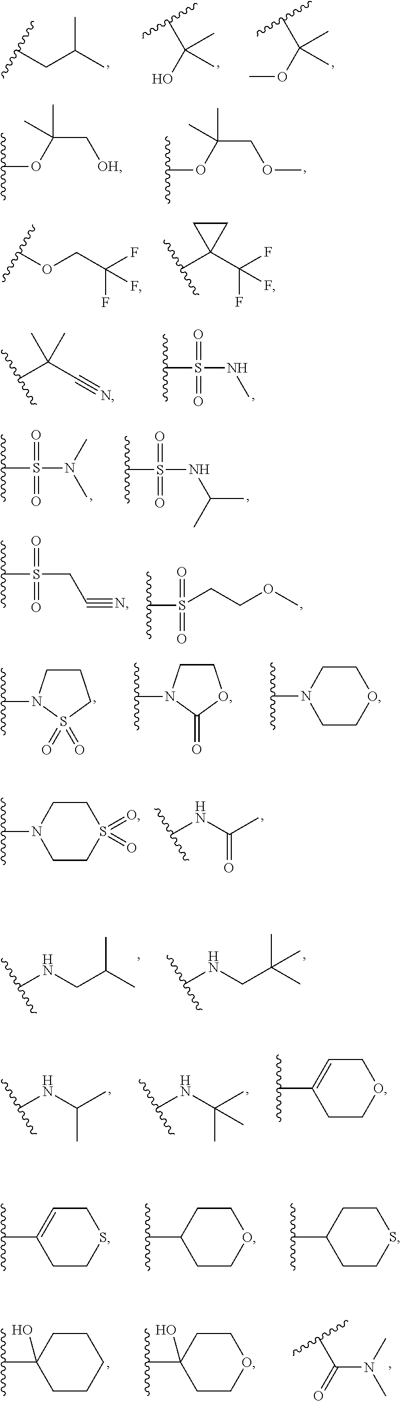

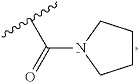 [SMILES: *C(=O)N1CCCC1]

trifluoromethyl, trifluoromethylthio, difluoromethoxy, and trifluoromethoxy; and

R₂₂ is selected from the group consisting of hydrogen, deuterium and methyl.

In further embodiments, R₇ is selected from the group consisting of hydrogen and C₁-C₃ alkyl.

In further embodiments, two of X₂, X₄, and X₅ are N, and one of X₂, X₄, and X₅ are O.

In further embodiments, one of X₂, X₄, and X₅ is N; one of X₂, X₄, is X₅ are O; and one of X₂, X₄, and X₅ is CH.

In certain embodiments, disclosed herein are compounds having structural Formula III

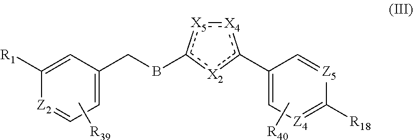

or a salt thereof, wherein:

B is selected from the group consisting of

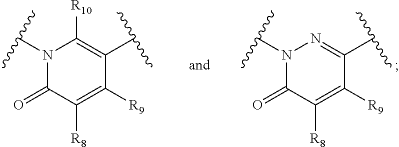

X₄ and X₅ are N and X₂ is O; X₂ and X₅ are N and X₄ is O; X₂ is CH, X₄ is N, and X₅ is O; X₂ is O, X₄ is CH, and X₅ is N; X₂ is CH, X₄ is O, and X₅ is N; X₂ is N, X₄ is CH, and X₅ is O;

Z₂ is selected from the group consisting of N and CR₁₄;

Z₄ is selected from the group consisting of N and CR₁₇;

Zₛ is selected from the group consisting of N and CR₁₉;

R₁ is selected from the group consisting of alkoxy, hydroxyalkyl, dihydroxylkyl, dialkylamidoalkyl, carboxylalkyl, hydroxyalkoxy, dihydroxyalkoxy, alkoxyalkoxy, alkylsulfonylalkoxy, dialkylamidoalkoxy, heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, oxoheterocycloalkyl, dialkylsulfonamido, and alkylsulfonyl, any of which may be optionally substituted with one or more substituents selected from the group consisting hydrogen, hydroxy, alkyl, hydroxyalkylcarbonyl, alkylsulfonyl, alkylsulfonamide, cyano, and oxo;

R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;

R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and

R₁₈ is selected from the group consisting of hydrogen, alkyl, hydroxyalkyl, hydroxyalkoxy, alkoxyalkyl, alkoxyalkoxy, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

In further embodiments, Z₄ and Z₅ are CH; and R₃₉ and R₄₀ are hydrogen.

In further embodiments, R₁ is selected from the group consisting of hydrogen, ethoxy, —SO₂CH₃, —SO₂CH₂CH₃,

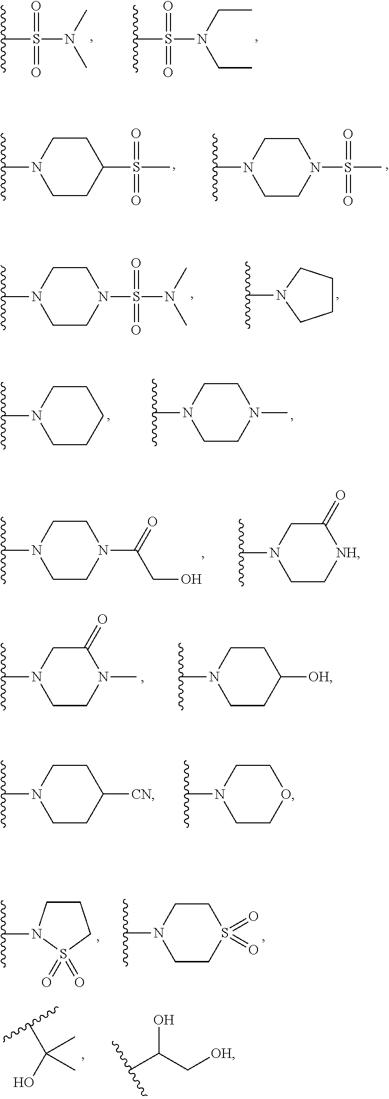

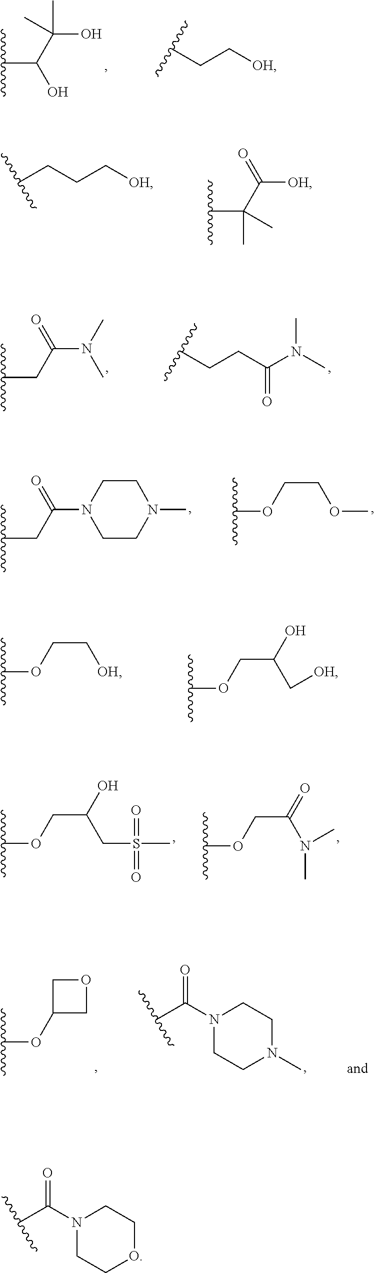

In further embodiments, R₁ is selected from the group consisting of hydrogen, ethoxy, —SO₂CH₃, —SO₂CH₂CH₃,

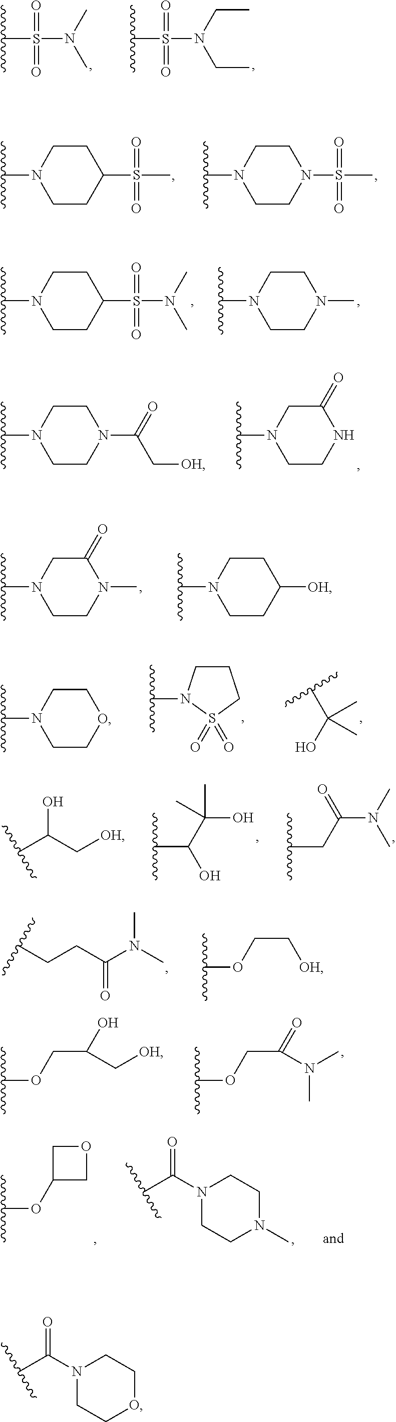

In further embodiments, wherein R₁ is selected from the group consisting of

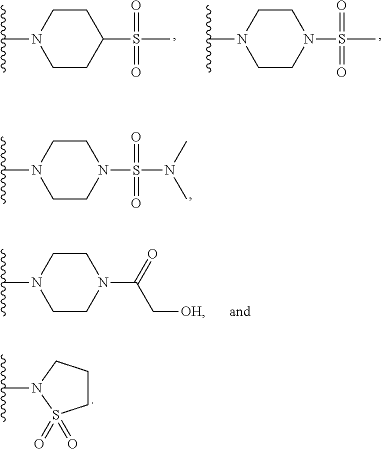

In further embodiments, R₁₈ is selected from the group consisting of hydrogen, isopropyl, tert-butyl, cyclopropyl, cyclohexyl, hydroxymethyl, isopropoxy, —SF₅, —SCF₃, —SO₂CH₃, —SO₂CHF₂, —SO₂CF₃,

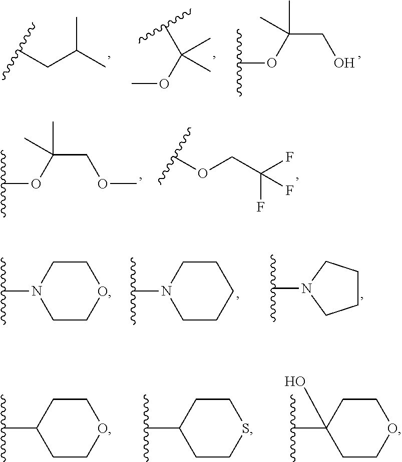

trifluoromethyl, difluoromethoxy, and trifluoromethoxy.

In further embodiments, R₁₈ is selected from the group consisting of hydrogen, isopropyl, tert-butyl, cyclopropyl, cyclohexyl, isopropoxy, —SO₂CH₃, —SO₂CHF₂, —SO₂CF₃,

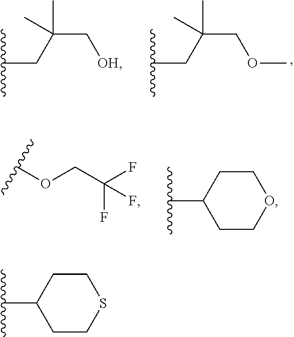

trifluoromethyl, difluoromethoxy, and trifluoromethoxy.

In further embodiments, R₁₈ is selected from the group consisting of isopropyl, tert-butyl, cyclopropyl, isopropoxy, —SO₂CH₃, —SO₂CF₃,

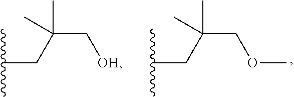

trifluoromethyl, difluoromethoxy, and trifluoromethoxy.

In certain embodiments, disclosed herein are compounds having structural Formula IV

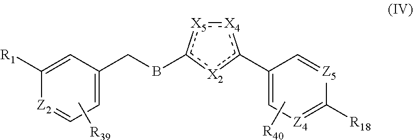

or a salt thereof, wherein:

B is selected from the group consisting of

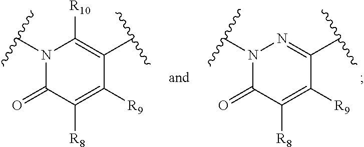

X₂ and X₄ are N and X₅ is O; X₄ and X₅ are N and X₂ is O; X₂ and X₅ are N and X₄ is O; X₂ is CH, X₄ is N, and X₅ is O; X₂ is CH, X₄ is O, and X₅ is N; X₂ is N, X₄ is CH, and X₅ is O;

Z₂ is selected from the group consisting of N and CR₁₄;

Z₄ is selected from the group consisting of N and CR₁₇;

Zₛ is selected from the group consisting of N and CR₁₉;

R₁ is selected from the group consisting of alkoxy, dialkylamidoalkyl, carboxylalkyl, hydroxyalkoxy, alkoxyalkoxy, alkylsulfonylalkoxy, dialkylamidoalkoxy, alkylsulfonylheterocycloalkyl, alkylsulfonamidoheterocycloalkyl, hydroxyalkylcarbonylheterocycloalkyl, oxoheterocycloalkyl, heterocycloalkylalkyl, heterocycloalkyloxy, dialkylsulfonamido, and alkylsulfonyl, any of which may be optionally substituted with one or more substituents selected from the group consisting hydrogen, hydroxy, alkyl, hydroxyalkylcarbonyl, alkylsulfonyl, alkylsulfonamide, cyano, and oxo;

R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;

R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and

R₁₈ is selected from the group consisting of alkyl, hydroxyalkyl, alkoxyalkyl, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

In further embodiments, Z₄ and Z₅ are CH; and R₁₃ and R₁₆ are hydrogen.

In further embodiments, R₁ is selected from the group consisting of hydrogen, ethoxy, —SO₂CH₃, —SO₂CH₂CH₃,

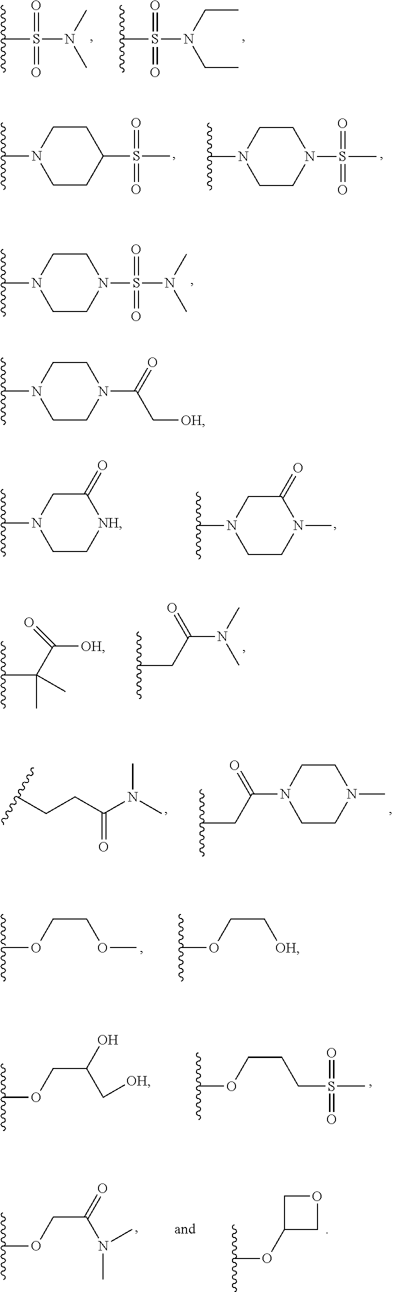

In further embodiments, R₁ is selected from the group consisting of hydrogen, ethoxy, —SO₂CH₃, —SO₂CH₂CH₃,

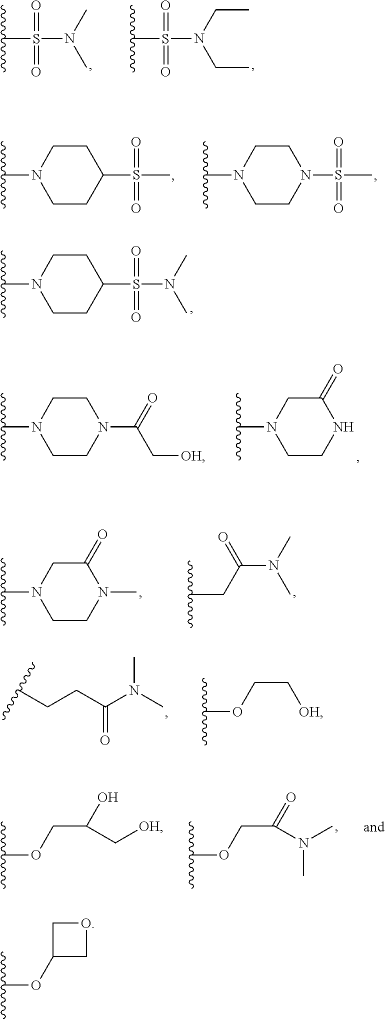

In further embodiments, R₁ is selected from the group consisting of

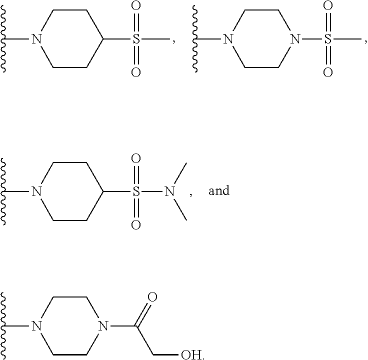

In further embodiments, R₁₈ is selected from the group consisting of hydrogen, isopropyl, tert-butyl, cyclopropyl, cyclohexyl, hydroxymethyl, isopropoxy, —SO₂CH₃, —SO₂CHF₂, —SO₂CF₃,

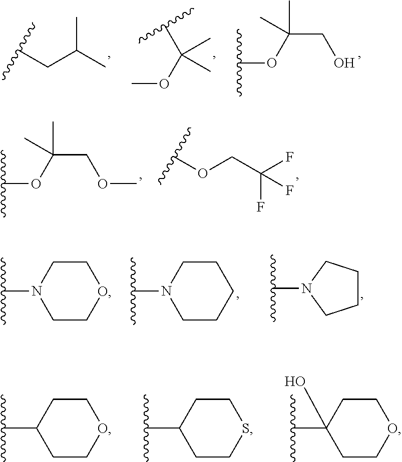

trifluoromethyl, difluoromethoxy, and trifluoromethoxy.

In further embodiments, R₁₈ is selected from the group consisting of hydrogen, isopropyl, tert-butyl, cyclopropyl, cyclohexyl, isopropoxy, —SO₂CH₃, —SO₂CHF₂, —SO₂CF₃,

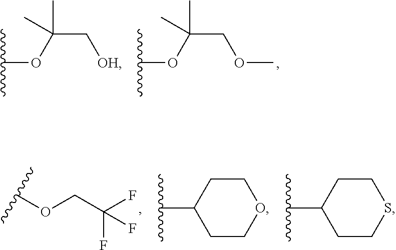

trifluoromethyl, difluoromethoxy, and trifluoromethoxy.

In further embodiments, R₁₈ is selected from the group consisting of isopropyl, tert-butyl, cyclopropyl, isopropoxy, —SO₂CH₃, —SO₂CF₃,

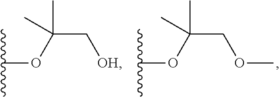

trifluoromethyl, difluoromethoxy, and trifluoromethoxy.

In certain embodiments, disclosed herein are compounds having structural Formula V

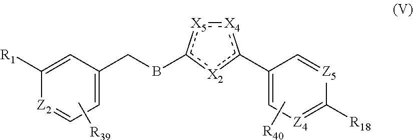

or a salt thereof, wherein:

B is selected from the group consisting of

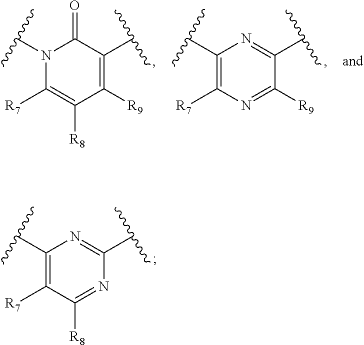

X₂ and X₄ are N and X₅ is O; X₄ and X₅ are N and X₂ is O; X₂ and X₅ are N and X₄ is O; X₂ is CH, X₄ is N, and X₅ is O; X₂ is CH, X₄ is O, and X₅ is N; X₂ is N, X₄ is CH, and X₅ is O;

Z₂ is selected from the group consisting of N and CR₁₄;

Z₄ is selected from the group consisting of N and CR₁₇;

Zₛ is selected from the group consisting of N and CR₁₉;

R₁ is selected from the group consisting of alkoxy, hydroxyalkyl, dihydroxylkyl, dialkylamidoalkyl, carboxylalkyl, hydroxyalkoxy, dihydroxyalkoxy, alkoxyalkoxy, alkylsulfonylalkoxy, dialkylamidoalkoxy, heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, oxoheterocycloalkyl, dialkylsulfonamido, and alkylsulfonyl, wherein said heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, and oxoheterocycloalkyl can be optionally substituted with one or more substituents selected from the group consisting hydrogen, hydroxy, alkyl, hydroxyalkylcarbonyl, alkylsulfonyl, alkylsulfonamide, cyano, and oxo;

R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;

R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and

R₁₈ is selected from the group consisting of hydrogen, alkyl, hydroxyalkyl, hydroxyalkoxy, alkoxyalkyl, alkoxyalkoxy, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

In further embodiments,

Z₄ and Z₅ are CH; and

R₁ is selected from the group consisting of hydrogen, ethoxy, —SO₂CH₃, —SO₂CH₂CH₃,

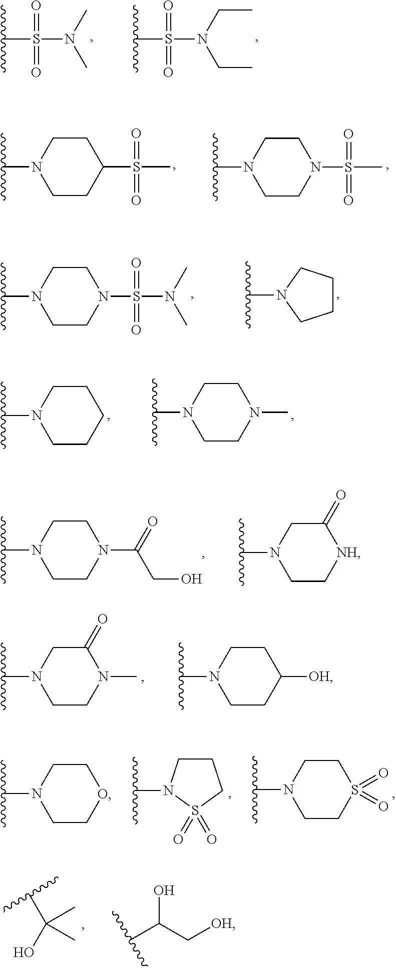

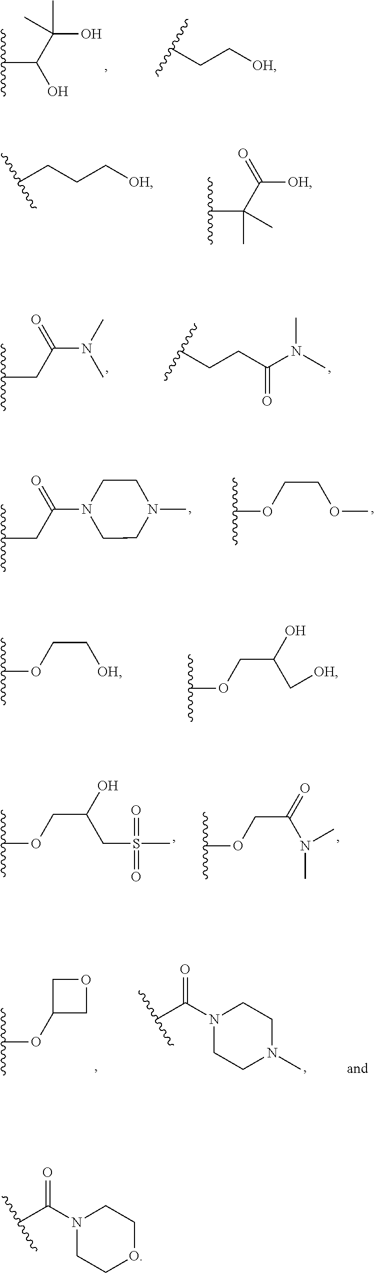

R₁₈ is selected from the group consisting of cyclopropyl, isopropoxy, and

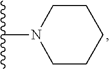 [SMILES: *N1CCCCC1]

and

R₃₉ and R₄₀ are hydrogen.

In certain embodiments, disclosed herein are compounds having structural Formula VI

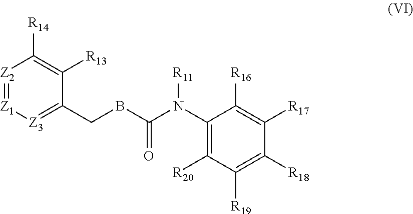

or a salt thereof, wherein:

B is selected from the group consisting of

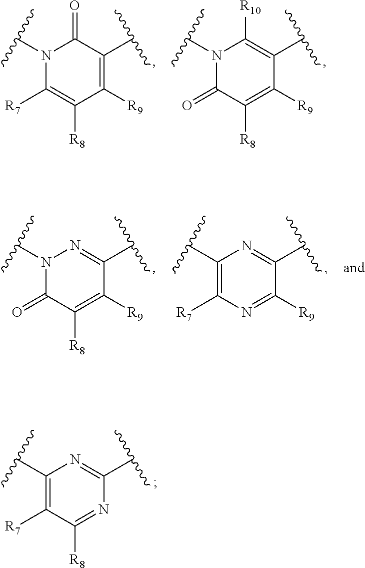

Z₁ and Z₂ are independently selected from the group consisting of N, NR₁, C═O, and CR₁;

Z₃ is selected from the group consisting of N, NR₁₂, C═O, and CR₁₂;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, carbonyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy,

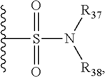

cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;

R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;

R₁₁ is selected from the group consisting of hydrogen, deuterium, alkyl, haloalkyl, perhaloalkyl, heteroalkyl, hydroxyalkyl, cycloalkyl, aryl, heterocycloalkyl, and heteroaryl;

R₁₂, R₁₃, and R₁₄ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted;

R₁₆, R₁₉, and R₂₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and cycloalkyl, any of which may be optionally substituted; and

R₁₇ and R₁₈ are independently selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted.

In further embodiments,

at least one of Z₁ or Z₂ is CR₁;

Z₃ is CR₁₂;

R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, acyl, carboxylalkyl, carboxyl, carbonyl, cyano, hydroxy, alkoxy, haloalkoxy, perhaloalkoxy, oxo, thiolalkyl, sulfonyl, sulfonamido, alkylsulfonyl, amino, amido, alkylamino, dialkylamino, nitro, heterocycloalkyl, heterocycloalkyloxy,

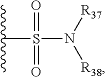

heterocycloalkylcarbonylalkyl, and heterocycloalkylcarbonyl, any of which can be optionally substituted with one or more substituents selected from the group consisting of hydrogen, deuterium, halogen, alkyl, alkenyl, amidoalkyl, acyl, carboxylalkyl, hydroxyalkylcarbonyl, alkynylcarbonyl, heteroalkyl, hydroxyalkyl, alkoxyalkyl, carboxyl, cyano, hydroxy, alkoxy, oxo, sulfonamido, alkylsulfonyl, amino, amido, carbamate, dialkylamino, dialkylaminoalkyl, trisubstituted siloxy, cycloalkyl, heterocycloalkyl, alkylheterocycloalkyl, any of which may be optionally substituted;

R₁₁ is selected from the group consisting of hydrogen, deuterium, alkyl, and cycloalkyl;

R₁₃ and R₁₄ are hydrogen; and

R₁₆, R₁₇, R₁₉, and R₂₀ are hydrogen.

In further embodiments, R₁₁ is hydrogen.

In further embodiments,

R₁ is selected from the group consisting of hydrogen, deuterium, fluorine, bromine, cyano, methyl, isopropyl,

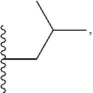 [SMILES: *CC(C)C]

ethylene,

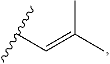 [SMILES: *C=C(C)C]

trifluoromethyl, bromomethyl, hydroxymethyl, difluoromethoxy, methoxy, ethoxy, isopropoxy, hydroxy, nitro, acetyl, carboxyl, —CO₂CH₃,

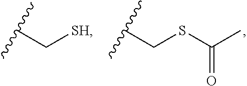

—SO₂CH₃, —SO₂CH₂CH₃, SO₂CH₂CH₂CH₃, —SO₂NH₂.

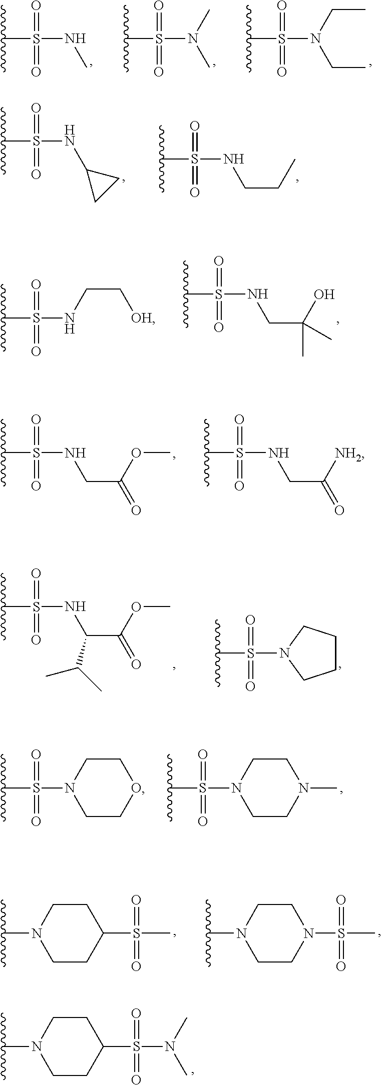

amino, methylamino, dimethylamino,

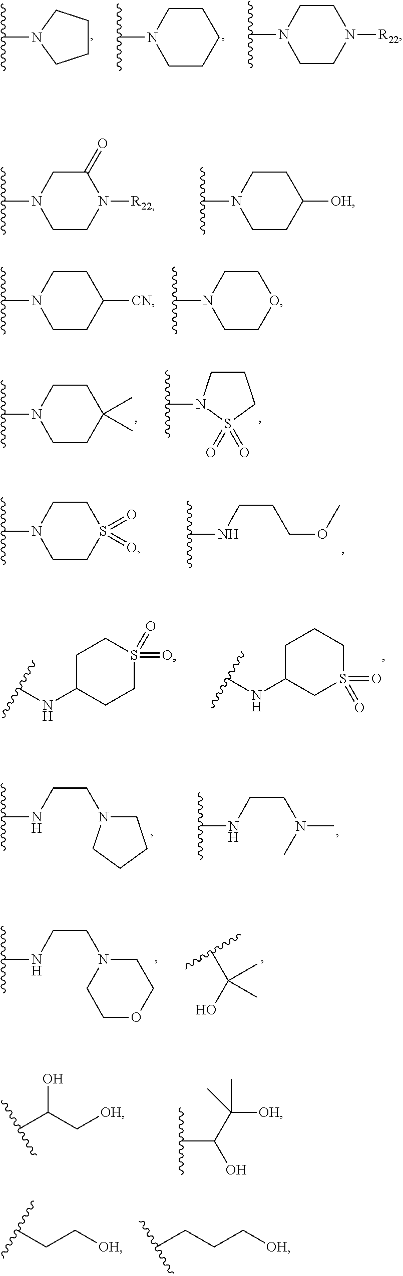

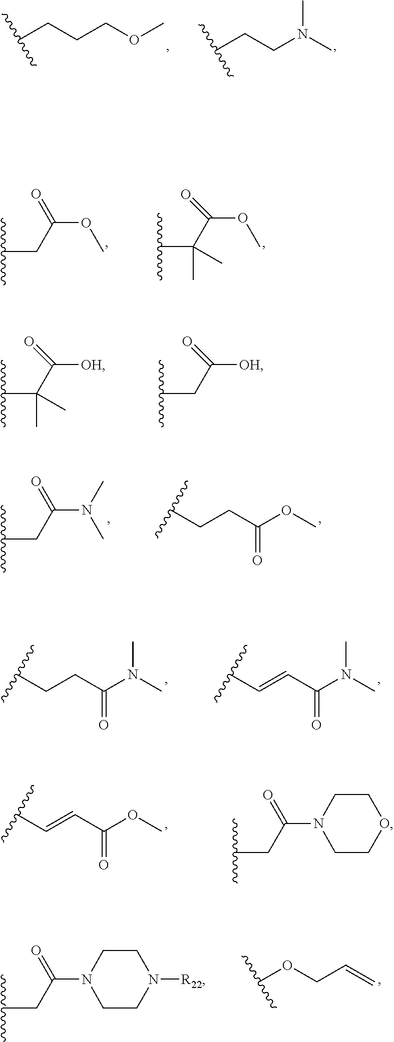

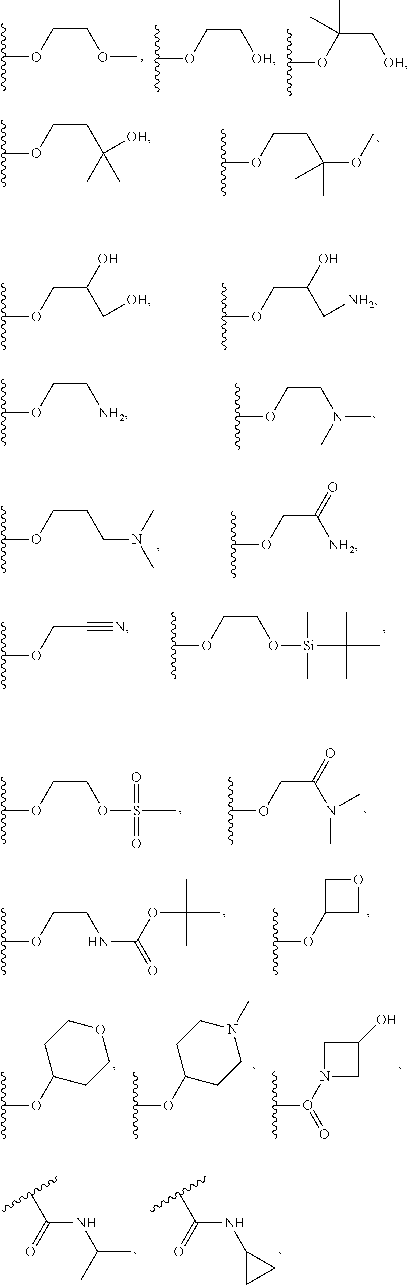

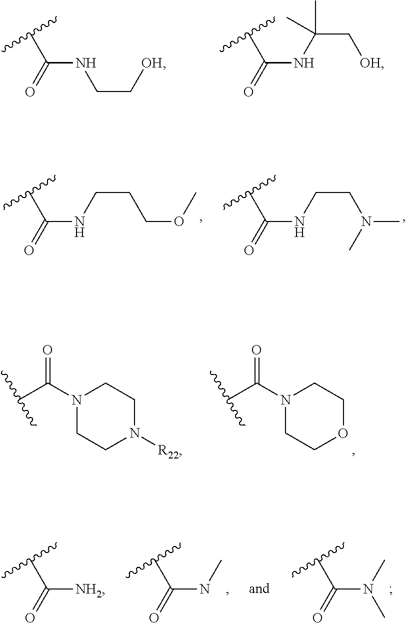

R₇, R₈, R₉, and R₁₀ are each independently selected from the group consisting of hydrogen, deuterium, C₁-C₃ alkyl, 4-pyridyl, and cyclopropyl;

R₁₈ is selected from the group consisting of hydrogen, deuterium, halogen, methyl, isopropyl, tert-butyl, cyclopropyl, cyclohexyl, acetyl, hydroxymethyl, methoxymethyl, methoxy, isopropoxy, methylamino, dimethylamino, methylthio, cyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃,

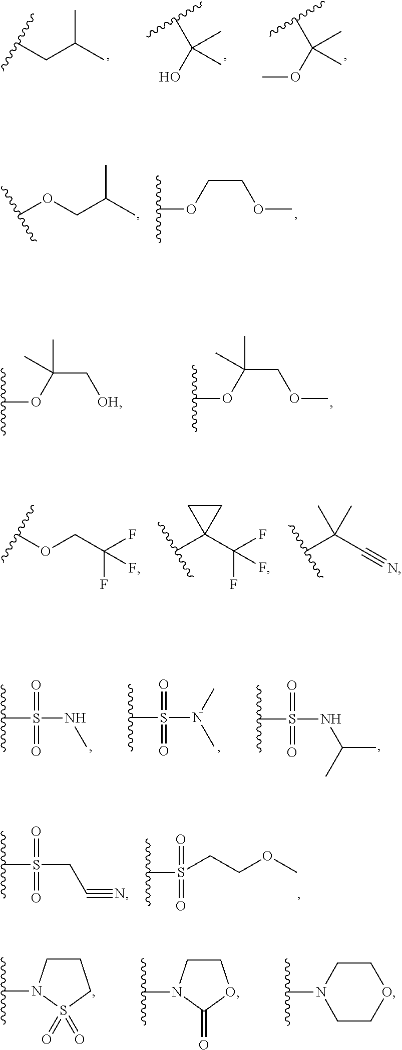

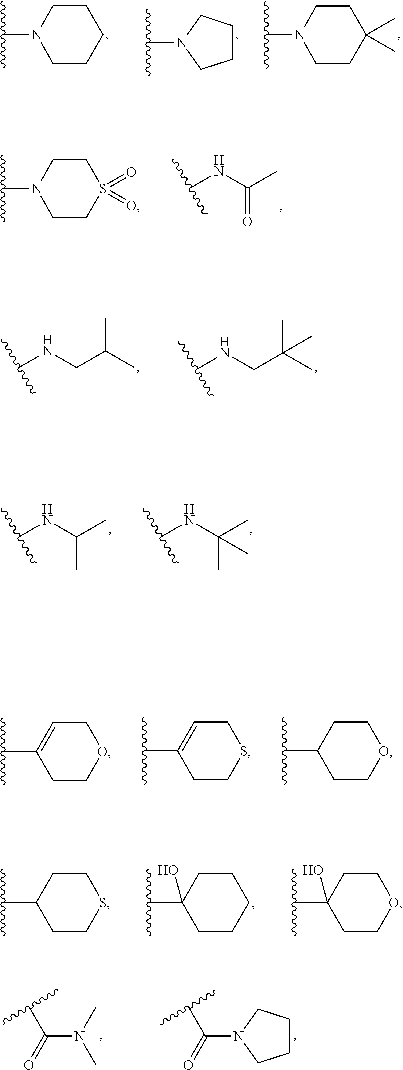

trifluoromethyl, trifluoromethylthio, difluoromethoxy, and trifluoromethoxy; and

R₂₂ is selected from the group consisting of hydrogen, deuterium, methyl, acetyl,

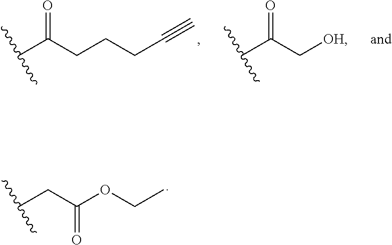

In further embodiments,

R₁ is selected from the group consisting of hydrogen, deuterium, chloro, cyano, methyl, ethylene,

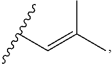 [SMILES: *C=C(C)C]

bromomethyl, hydroxymethyl, difluoromethoxy, methoxy, ethoxy, isopropoxy, hydroxy, nitro, —CO₂CH₃,

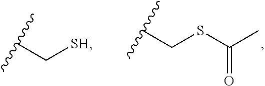

—SO₂CH₃, —SO₂CH₂CH₃, SO₂CH₂CH₂CH₃, —SO₂NH₂,

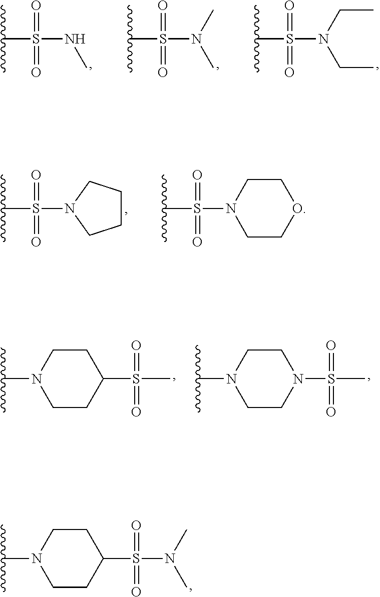

dimethylamino,

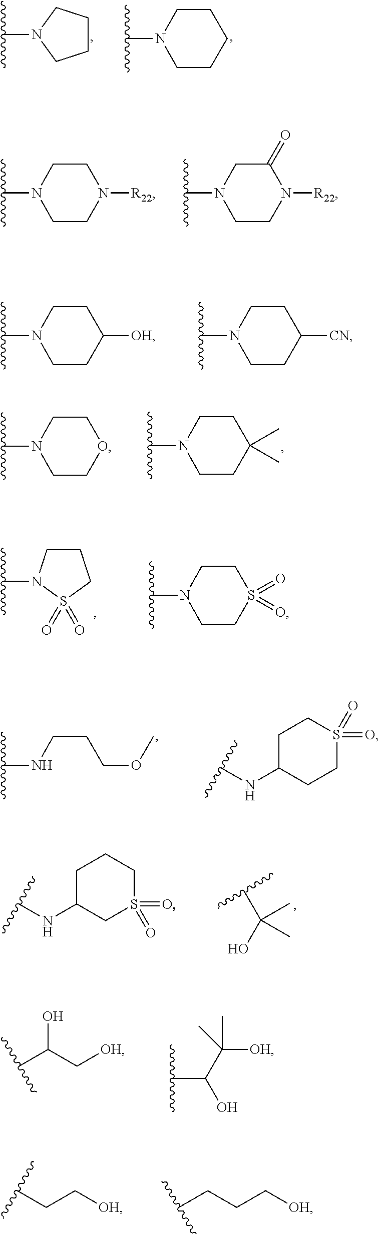

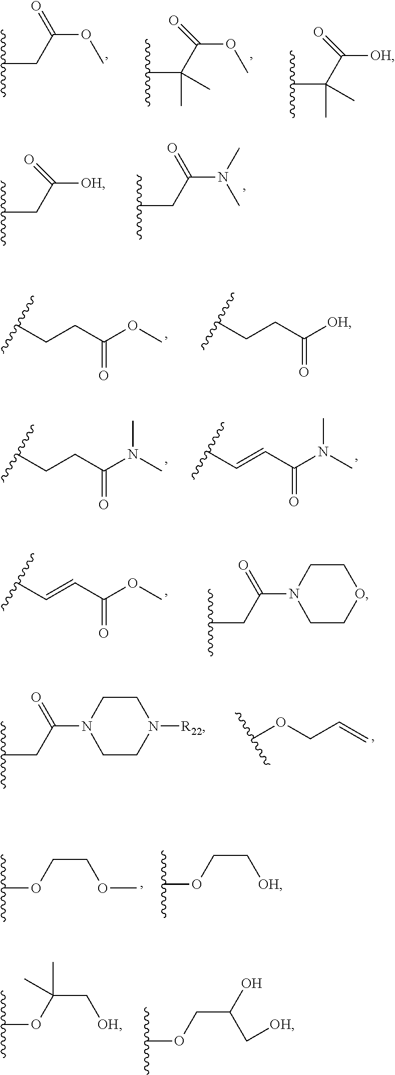

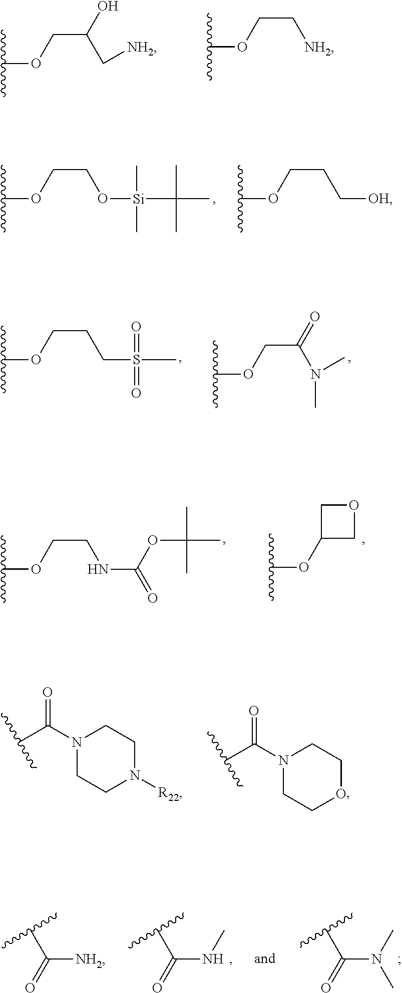

R₇, R₈, R₉, and R₁₀ are each independently selected from the group consisting of hydrogen, deuterium and methyl;

R₁₈ is selected from the group consisting of hydrogen, deuterium, chloro, methyl, isopropyl, tert-butyl, cyclopropyl, cyclohexyl, acetyl, hydroxymethyl, methoxymethyl, methoxy, isopropoxy, methylamino, dimethylamino, methylthio, cyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃,

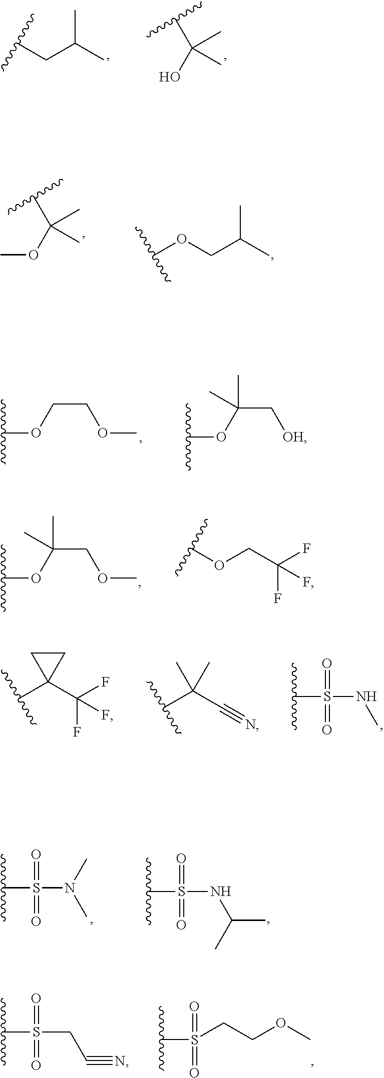

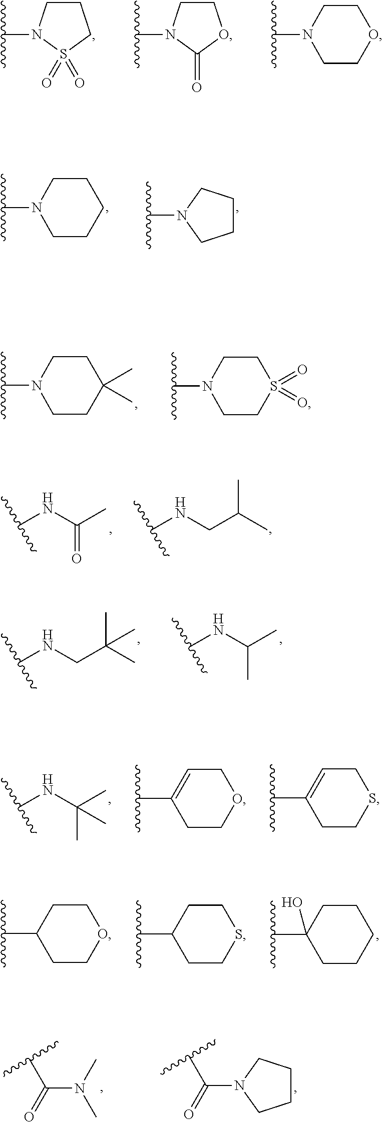

trifluoromethyl, trifluoromethylthio, difluoromethoxy, and trifluoromethoxy; and

R₂₂ is selected from the group consisting of hydrogen, deuterium and methyl.

In further embodiments, R₇ is C₁-C₃ alkyl.

In further embodiments, R₁₁ is hydrogen.

In certain embodiments, disclosed herein are compounds having structural Formula VII

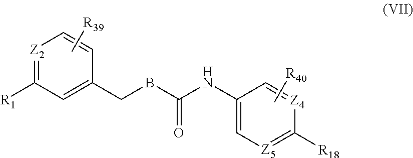

or a salt thereof, wherein:

B is selected from the group consisting of

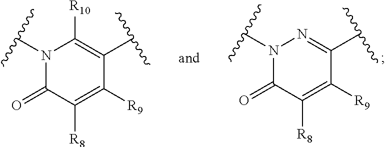

Z₁ is selected from the group consisting of N and CR₁₄;

Z₄ is selected from the group consisting of N and CR₁₇;

Zₛ is selected from the group consisting of N and CR₁₉;

R₁ is selected from the group consisting of alkoxy, hydroxyalkyl, dialkylamidoalkyl, carboxylalkyl, hydroxyalkoxy, alkoxyalkoxy, alkylsulfonylalkoxy, dialkylamidoalkoxy, heterocycloalkyl, heterocycloalkylalkyl, heterocycloalkyloxy, heterocycloalkylcarbonyl, dialkylsulfonamido, and alkylsulfonyl, wherein said heterocycloalkyl, heterocycloalkylalkyl, heterocycloalkyloxy, heterocycloalkylcarbonyl can be optionally substituted with one or more substituents selected from the group consisting hydrogen, hydroxy, alkyl, hydroxyalkylcarbonyl, alkylsulfonyl, alkylsulfonamide, and oxo;

R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and

R₁₈ is selected from the group consisting of alkyl, hydroxyalkyl, alkoxyalkyl, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, and hydroxyheterocycloalkyl.

In further embodiments, Z₄ and Z₅ are CH; and R₁₃ and R₁₆ are hydrogen.

In further embodiments, R₁ is selected from the group consisting of hydrogen, ethoxy, —SO₂CH₃, —SO₂CH₂CH₃,

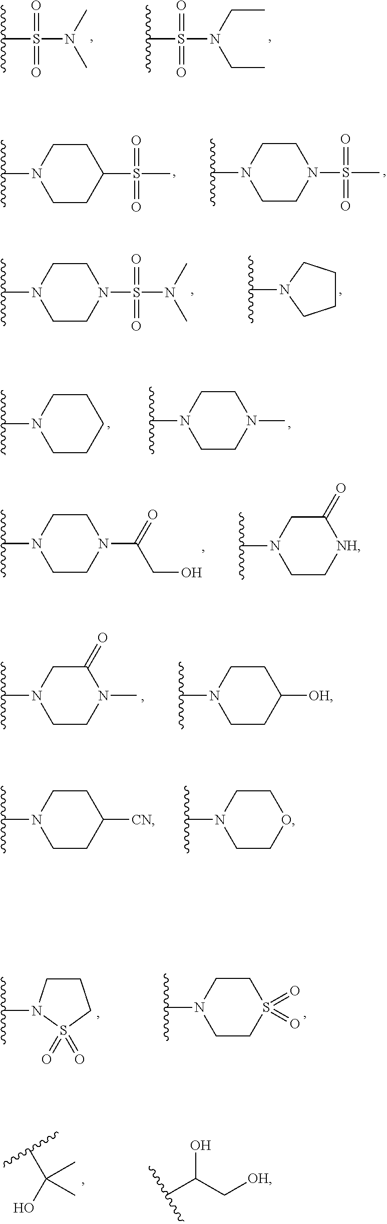

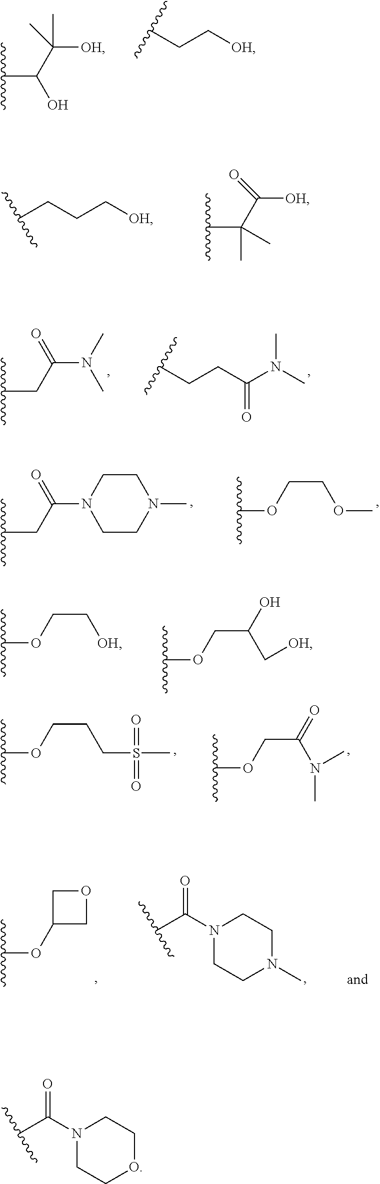

In further embodiments, R₁ is selected from the group consisting of hydrogen, ethoxy, —SO₂CH₃, —SO₂CH₂CH₃,

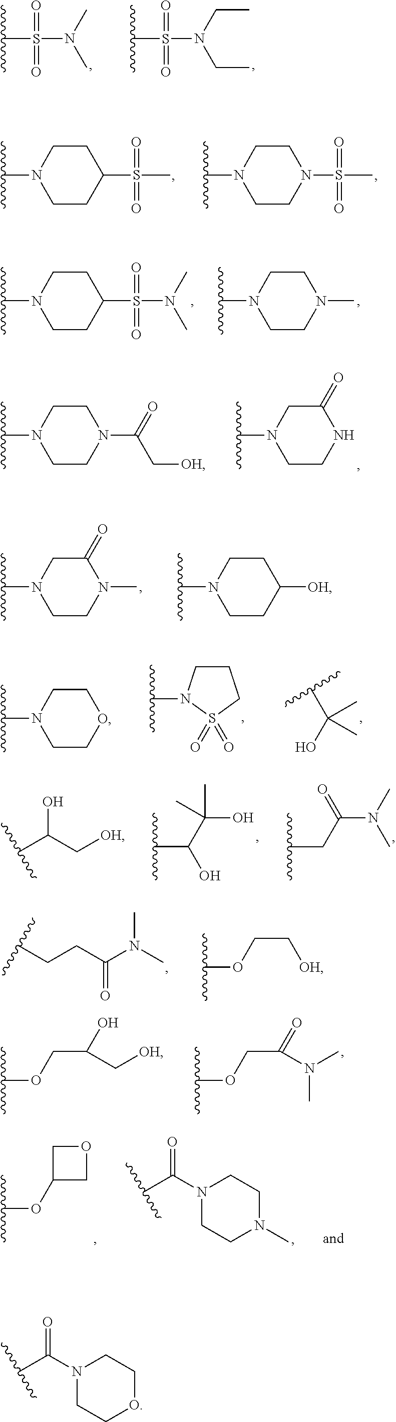

In further embodiments, R₁ is selected from the group consisting of

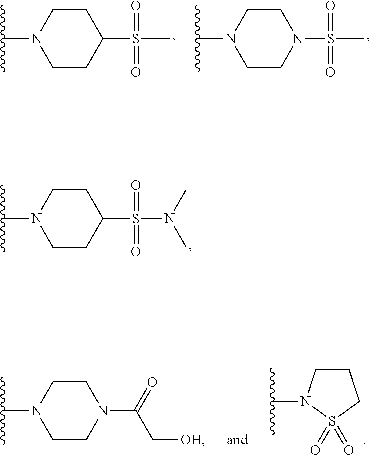

In further embodiments, R₁₈ is selected from the group consisting of hydrogen, isopropyl, tert-butyl, cyclopropyl, cyclohexyl, hydroxymethyl, isopropoxy, —SO₂CH₃, —SO₂CHF₂, —SO₂CF₃,

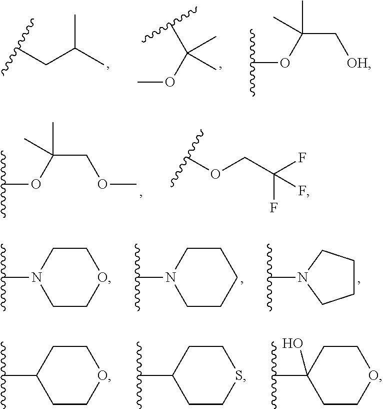

trifluoromethyl, difluoromethoxy, and trifluoromethoxy.

In further embodiments, R₁₈ is selected from the group consisting of hydrogen, isopropyl, tert-butyl, cyclopropyl, cyclohexyl, isopropoxy, —SO₂CH₃, —SO₂CHF₂, —SO₂CF₃,

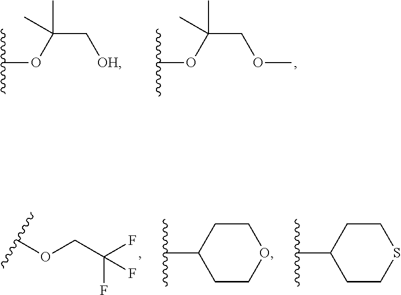

trifluoromethyl, difluoromethoxy, and trifluoromethoxy.

In further embodiments, R₁₈ is selected from the group consisting of isopropyl, tert-butyl, cyclopropyl, isopropoxy, —SO₂CH₃, —SO₂CF₃,

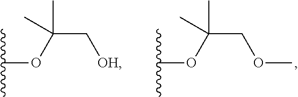

trifluoromethyl, difluoromethoxy, and trifluoromethoxy.

In further embodiments, disclosed herein is a compound selected from the group consisting of Examples 1 to 23, 25, 27 to 106, 108, 111 to 113, 116 to 132, 135 to 152, and 154 to 270, or a salt thereof.

In further embodiments, disclosed herein is a pharmaceutical composition comprising a compound as disclosed herein together with a pharmaceutically acceptable carrier.

In further embodiments, disclosed herein is a method of treatment of a HIF pathway-mediated disease comprising the administration of a therapeutically effective amount of a compound as disclosed herein to a patient in need thereof.

In further embodiments, said disease is cancer.

In further embodiments, said cancer is selected from the group consisting of colon cancer, breast cancer, ovarian cancer, lung cancer, prostrate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease, non-Hodgkin's lymphomas, multiple myeloma, hematopoietic malignancies including leukemias (Chronic Lymphocytic Leukemia (CLL), Acute Lymphocytic Leukemia (ALL)) and lymphomas including lymphocytic, granulocytic and monocytic; adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In further embodiments, disclosed herein is a method of treatment of a disease caused by abnormal cell proliferation comprising the administration of a therapeutically effective amount of a compound as disclosed herein to a patient in need thereof.

In further embodiments, disclosed herein is a method of treatment of a HIF pathway-mediated disease comprising the administration of:

a. a therapeutically effective amount of a compound as disclosed herein; and

b. another therapeutic agent.

In further embodiments, disclosed herein is a method for achieving an effect in a patient comprising the administration of a therapeutically effective amount of a compound as disclosed herein to a patient, wherein the effect is selected from the group consisting of preventing or reducing resistance to radiotherapy and chemotherapy, preventing or reducing tumor invasion and tumor metastasis, and preventing or reducing angiogenesis.

In certain embodiments, the compositions and methods disclosed herein may be used to inhibit HIF pathway activity, to downregulate HIF-1α (which is induced by hypoxia or genetic alterations, as well as in various disease states, e.g. in persons with certain genetic backgrounds), by increasing HIF-1α degradation, decreasing HIF heterodimer formation, increasing HIF-1α prolyl hydroxylation, and/or to reduce transcription of hypoxia response element (HRE) downstream elements.

In certain embodiments, the compositions and methods disclosed herein may be used to reduce tumor growth, to inhibit neoangiogenesis (e.g., by downregulating VEGF), to normalize tumor vasculature, to enhance radiotherapy and chemotherapy, to prevent metastasis, to reduce tumor stem cell numbers, and to prevent induction of anaerobic cellular metabolism.

In certain embodiments, the compositions and methods disclosed herein may be used to treat HIF-deregulated diseases with an inflammatory component, such as cancers, stroke, and rheumatoid arthritis.

In certain embodiments, the compositions and methods disclosed herein may be used to treat HIF-deregulated diseases cardiovascular diseases such as cardiac arrhythmia and heart failure.

In certain embodiments, the compositions and methods disclosed herein are useful for preventing or reducing resistance to radiotherapy and chemotherapy.

In certain embodiments, the compositions and methods disclosed herein are useful for preventing or reducing tumor invasion and tumor metastasis.

In certain embodiments, the compositions and methods disclosed herein are useful for preventing or reducing angiogenesis and disorders related to angiogenesis.

In certain embodiments, the compounds disclosed herein may be used as a medicament.

In further embodiments, said compounds which may be used as a medicament include the compounds of Formula I, II, III, IV, V, VI, and VII, optionally including any further limitation to the scope of said Formulas as defined above. In further embodiments, said compounds may be selected from the group consisting of Examples 1 to 163, or a salt thereof.

In certain embodiments, the disclosed are compounds for use in the treatment of a HIF pathway-mediated disease.

In further embodiments, said compounds which may be used in the treatment of a HIF pathway-mediated disease include the compounds of Formula I, II, III, IV, V, VI, and VII, optionally including any further limitation to the scope of said Formulas as defined above. In further embodiments, said compounds may be selected from the group consisting of Examples 1 to 23, 25, 27 to 106, 108, 111 to 113, 116 to 132, 135 to 152, and 154 to 270, or a salt thereof.

In further embodiments, said disease is cancer.

In further embodiments, said cancer is selected from the group consisting of colon cancer, breast cancer, ovarian cancer, lung cancer, prostrate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease, non-Hodgkin's lymphomas, multiple myeloma, hematopoietic malignancies including leukemias (Chronic Lymphocytic Leukemia (CLL), Acute Lymphocytic Leukemia (ALL)) and lymphomas including lymphocytic, granulocytic and monocytic; adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In certain embodiments, disclosed herein are compounds for use in the treatment of a disease caused by abnormal cell proliferation.

In certain embodiments, disclosed herein are compounds for use in the treatment of HIF-deregulated diseases with an inflammatory component, such as cancers, stroke, and rheumatoid arthritis.

In certain embodiments, disclosed herein are compounds for use in the treatment of HIF-deregulated cardiovascular diseases such as cardiac arrhythmia and heart failure.

In certain embodiments, disclosed herein are compounds for use in the treatment of preventing or reducing resistance to radiotherapy and chemotherapy.

In certain embodiments, disclosed herein are compounds for use in the prevention or reduction of tumor invasion and tumor metastasis.

In certain embodiments, disclosed herein are compounds for use in the prevention or reduction of angiogenesis and disorders related to angiogenesis.

In further embodiments, said compounds which may be used in the treatment of a disease caused by abnormal cell proliferation, HIF-deregulated diseases with an inflammatory component, such as cancers, stroke, and rheumatoid arthritis, HIF-deregulated cardiovascular diseases such as cardiac arrhythmia and heart failure, for use in preventing or reducing resistance to radiotherapy and chemotherapy, prevention or reduction of tumor invasion and tumor metastasis, or prevention or reduction of angiogenesis and disorders related to angiogenesis include the compounds of Formula I, II, III, IV, V, VI, and VII, optionally including any further limitation to the scope of said Formulas as defined above. In further embodiments, said compounds may be selected from the group consisting of Examples 1 to 23, 25, 27 to 106, 108, 111 to 113, 116 to 132, 135 to 152, and 154 to 270, or a salt thereof.

### BRIEF DESCRIPTION OF THE DRAWINGS

FIG. 1—Compounds of this invention inhibit the growth of diffuse large B-cell lymphoma TMD8 cells as shown by reduced number of viable cells following treatment with Example 7.

FIG. 2—Compounds of this invention inhibit the growth of neuroblastoma NB-1 cells (FIG. 2a) and D423 cells (FIG. 2b) as shown by reduced number of viable cells following treatment with Example 7.

FIG. 3—Compounds of this invention inhibit the growth of glioblastoma Gli56 cells as shown by reduced number of viable cells following treatment with Example 7.

FIG. 4—Compounds of this invention inhibit the growth of NB-1 xenografts in vivo, daily oral treatment with 40 mg/kg of Example 7 reduces the tumor growth.

FIG. 5—Compounds of this invention inhibit the growth of H460 xenografts in vivo, daily oral treatment with 40 mg/kg of Example 7 reduces the tumor growth.

FIG. 6—Compounds of this invention reduce the level of hypoxia in H460 xenografts, daily oral treatment with 40 mg/kg of Example 7 reduce the level of hypoxia as measure by hypoxyprobe.

FIG. 7—Compounds of this invention reduce the level of the HIF regulated gene carbonic anhydrase IX in H460 xenografts, daily oral treatment with 40 mg/kg of Example 7 reduce the level of CAIX as shown by IHC.

FIG. 8—Compounds of this invention inhibit the growth of leukemia OCI-AML3 cells as shown by reduced number of viable cells following treatment with Example 7.

FIG. 9—Compounds of this invention inhibit the growth of leukemia CD45+ primary AML cells from an AML patient as shown by reduced number of viable cells following treatment with Example 7 (FIG. 9a), and increase the increase the number of apoptotic Annexin V positive cells (FIG. 9b), while CD45+ normal bone marrow cells from a healthy volunteer show minimal response to Example 7.

FIG. 10—Compounds of this invention reduce disease burden in human leukemia model, daily oral treatment with 60 mg/kg of Example 7 reduces disease burden in OCI-AML3 models in NSG mice as measured by IVIS imaging.

FIG. 11—Compounds of this invention prolong the survival in human leukemia model, daily oral treatment with 60 mg/kg of Example 7 extends survival in OCI-AML3 models in NSG mice.

FIG. 12—Compounds of this invention enhance fractionated irradidation in head and neck xenograft model-experimental design.

FIG. 13—Compounds of this invention enhance fractionated irradidation in head and neck xenograft model-tumor diameter of HN5 xenograft following daily oral treatment with 60 mg/kg of Example 7 and/or 4 Gy dose of irradiation for 5 days.

Unless otherwise defined, all technical and scientific terms used herein have the same meaning as commonly understood by one of ordinary skill in the art to which this invention belongs. When a conflict occurs, the meaning ascribed herein controls.

When ranges of values are disclosed, and the notation “from n1 . . . to n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)—group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to—NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type, including spiro-ring fused systems. The bicyclic and tricyclic types of isomer are exemplified in general by, bicyclo[1,1,1]pentane, camphor, adamantane, bicyclo[3,2,1]octane, and [4,4.1]-bicyclononane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂ —), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, cyclic sulfonamides, sulfoxides, N-oxides of tertiary nitrogen ring members, and carbocyclic fused, benzo fused, and spiro-ring fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, isothiazolidine, and the like. The heterocycle groups may be optionally substituted unless specifically prohibited.

The term “hydrogen,” as used herein, refers to both protium (¹H) and deuterium (²H). This definition extends to hydrogen atoms which appear in chemical structural drawings disclosed herein, including at sites where hydrogen atoms are not explicitly shown. For example, a chemical structure disclosed herein may include an ethyl group represented as

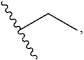 [SMILES: *CC]

which includes five hydrogen atoms which are not explicitly drawn, any of which can be protium (¹H) or deuterium (²H). This definition also extends to hydrogen atoms which form a part of a named chemical substituent disclosed herein. For example, a generic chemical structure disclosed herein may recite an aryl group, which encompasses specific embodiments such as a phenyl group, which comprises five hydrogen atoms, any of which can be protium (¹H) or deuterium (²H).

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

In certain embodiments, compounds disclosed herein are enriched with deuterium. Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate, including cases where a C—H bond is broken during metabolism of a compound disclosed herein. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. The deuteration approach has the potential to slow the metabolism of the compounds disclosed herein. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. Deuterium can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques where deuterium is directly and specifically inserted by a deuterated reagent of known isotopic content, can yield high deuterium abundance, but can be limited by the chemistry required. Exchange techniques, on the other hand, may yield lower deuterium incorporation, often with the isotope being distributed over many sites on the molecule.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position. When a group in a chemical formula is designated to be “a bond,” the group reduces to a linkage between the groups to which it is linked in the formula. By way of example, in Formula I, when Y₂ is a bond, it becomes a direct link between A and -alkyl-N(R₄)R₅, forming R₅(R₄)N-alkyl-A-Y₁—(B—(R₂)ₘ)-D-E-(R₃)ₚ.

As used herein, the term “modulate” means to increase or decrease the activity of a target or the amount of a substance.

As used herein, the term “increase” or the related terms “increased,” “enhance” or “enhanced” refers to a statistically significant increase, and the terms “decreased,” “suppressed,” or “inhibited” to a statistically significant decrease. For the avoidance of doubt, an increase generally refers to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control, baseline, or prior-in-time value. Inhibition generally refers to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease over the control value.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single formulation (e.g., a capsule or injection) having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, polymorphs, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis.

In the present invention, the term “radiation” means ionizing radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art. The amount of ionizing radiation needed in a given cell generally depends on the nature of that cell. Means for determining an effective amount of radiation are well known in the art. Used herein, the term “an effective dose” of ionizing radiation means a dose of ionizing radiation that produces an increase in cell damage or death.

The term “radiation therapy” refers to the use of electromagnetic or particulate radiation in the treatment of neoplasia and includes the use of ionizing and non-ionizing radiation.

As used herein, the term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound and N-oxides of amines or heterocyclic groups such as pyridine.

The term “metabolite” refers to a compound produced through biological transformation of a compound following administration to a subject. In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C)-bond, N-oxidation, or covalent bonding of a polar molecule or functional group (such as sulfate, glucuronic acid, glutathione, or glycine, to the therapeutic agent. The resultant metabolites may be stable or unstable under physiological conditions, and can have substantially different pharmacokinetic, pharmacodynamic, and acute and long-term toxicity profiles relative to the parent compounds. Certain compounds disclosed herein may, after administration to a subject result in formation of metabolites, which in some cases have biological activity as HIF pathway modulators or activity against other biological systems. In certain embodiments, metabolites of the compounds disclosed herein include N-oxides, particularly N-oxides of heterocyclic groups such as pyridine. In further embodiments, metabolites of compounds disclosed herein may themselves have substantial activity as HIF pathway inhibitors.

The compounds disclosed herein can exist as therapeutically acceptable salts. Suitable acid addition salts include those formed with both organic and inorganic acids, and will normally be pharmaceutically acceptable. However, salts of non-pharmaceutically acceptable salts may be of utility in the preparation and purification of the compound in question. Basic addition salts may also be formed and be pharmaceutically acceptable. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion.

Basic addition salts can be prepared during the final isolation and purification of the compounds, often by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium (e.g., NaOH), potassium (e.g., KOH), calcium (including Ca(OH)₂), magnesium (including Mg(OH)₂ and magnesium acetate), zinc, (including Zn(OH)₂ and zinc acetate) and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-diethylethanolamine, N,N′-dimethylethanolamine, triethanolamine, and tromethamine. Basic amino acids such as 1-glycine and 1-arginine, and amino acids which may be zwitterionic at neutral pH, such as betaine (N,N,N-trimethylglycine) are also contemplated.

Salts disclosed herein may combine in 1:1 molar ratios, and in fact this is often how they are initially synthesized. However, it will be recognized by one of skill in the art that the stoichiometry of one ion in a salt to the other may be otherwise. Salts shown herein may be, for the sake of convenience in notation, shown in a 1:1 ratio; all possible stoichiometric arrangements are encompassed by the scope of the present invention.

The terms, “polymorphs” and “polymorphic forms” and related terms herein refer to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing, for example, one polymorph might be more likely to form solvates or might be difficult to filter and wash free of impurities (i.e., particle shape and size distribution might be different between polymorphs).

Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

While it may be possible for the compounds and prodrugs disclosed herein to be administered as the raw chemical, it is also possible to present them as a pharmaceutical formulation. Accordingly, provided herein are pharmaceutical formulations which comprise one or more of certain compounds and prodrugs disclosed herein, or one or more pharmaceutically acceptable salts, esters, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds and prodrugs disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds and prodrugs may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds and prodrugs may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds and prodrugs which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds and prodrugs to allow for the preparation of highly concentrated solutions.

In addition to the formulations described previously, a compound or prodrug as disclosed herein may also be formulated as a depot preparation. Such long acting formulations may be administered by implantation (for example subcutaneously or intramuscularly) or by intramuscular injection. Thus, for example, the compounds and prodrugs may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds and prodrugs may also be formulated in rectal compositions such as suppositories or retention enemas, e.g., containing conventional suppository bases such as cocoa butter, polyethylene glycol, or other glycerides.

Certain compounds and prodrugs disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds and prodrugs may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds and prodrugs disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors. In humans, intranasal insulin has been shown to improve memory in normal adults and patients with Alzheimer's disease. Hanson L R and Frey W H, 2nd, J Neuroimmune Pharmacol. 2007 March; 2(1):81-6. Epub 2006 Sep. 15.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds and prodrugs may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compound or prodrug which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds and prodrugs can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds and prodrugs described herein (or a pharmaceutically acceptable salt or ester thereof) in combination with another therapeutic agent. By way of example only, if one of the side effects experienced by a patient upon receiving one of the compounds herein for the treatment of cancer is nausea, then it may be appropriate to administer an antiemetic agent in combination. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

The compounds disclosed herein, including compounds of Formula I, are also useful as chemo- and radio-sensitizers for cancer treatment. They are useful for the treatment of mammals who have previously undergone or are presently undergoing or will be undergoing treatment for cancer. Such other treatments include chemotherapy, radiation therapy, surgery or immunotherapy, such as cancer vaccines.

The instant compounds are particularly useful in combination with therapeutic, anti-cancer and/or radiotherapeutic agents. Thus, the present invention provides a combination of the presently compounds of Formula I with therapeutic, anti-cancer and/or radiotherapeutic agents for simultaneous, separate or sequential administration. The compounds of this invention and the other anticancer agent can act additively or synergistically. A synergistic combination of the present compounds and another anticancer agent might allow the use of lower dosages of one or both of these agents and/or less frequent dosages of one or both of the instant compounds and other anticancer agents and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

The therapeutic agent, anti-cancer agent and/or radiation therapy can be administered according to therapeutic protocols well known in the art. It will be apparent to those skilled in the art that the administration of the therapeutic agent, anti-cancer agent and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse affects.

Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Any suitable means for delivering radiation to a tissue may be employed in the present invention. Common means of delivering radiation to a tissue is by an ionizing radiation source external to the body being treated. Alternative methods for delivering radiation to a tissue include, for example, first delivering in vivo a radiolabeled antibody that immunoreacts with an antigen of the tumor, followed by delivering in vivo an effective amount of the radio labeled antibody to the tumor. In addition, radioisotopes may be used to deliver ionizing radiation to a tissue or cell. Additionally, the radiation may be delivered by means of a radiomimetic agent. As used herein a “radiomimetic agent” is a chemotherapeutic agent, for example melphalan, that causes the same type of cellular damage as radiation therapy, but without the application of radiation.

In one embodiment, the compounds of formula I can be administered in combination with one or more agent selected from aromatase inhibitors, antiestrogens, anti-progesterons, anti-androgens, or gonadorelin agonists, anti-inflammatory agents, antihistamines, anti-cancer agent, inhibitors of angiogenesis, topoisomerase 1 and 2 inhibitors, microtubule active agents, alkylating agents, antineoplastic, antimetabolite, dacarbazine (DTIC), platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, HSP90 inhibitor, smoothened antagonist, inhibitors of Ras isoforms, telomerase inhibitors, protease inhibitors, metalloproteinase inhibitors, aminopeptidase inhibitors, imununomodulators, therapeutic antibody and a protein kinase inhibitor, e.g., a tyrosine kinase or serine/threonine kinase inhibitor.

In another embodiment is provided a combination of a compound of formula I and an anti-cancer agent for simultaneous, separate or sequential administration.

Examples of cancer agents or chemotherapeutic agents for use in combination with the compounds as disclosed herein can be found in Cancer Principles and Practice of Oncology by V. T. Devita and S. Hellman (editors), 6th edition (Feb. 15, 2001), Lippincott Williams & Wilkins Publishers, and WO 2006/061638. A person of ordinary skill in the art would be able to discern which combinations of agents would be useful based on the particular characteristics of the drugs and the cancer involved. Classes of such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs), agents that interfere with cell cycle checkpoints, PARP inhibitors, HDAC inhibitors, Smo antagonists (HH inhibitors), HSP90 inhibitors, CYP17 inhibitors, 3rd generation AR antagonists, JAK inhibitors e.g. Ruxolitinib (trade name Jakafi, and BTK kinase inhibitors.

Anticancer agents suitable for use in the combination therapy with compounds as disclosed herein include, but are not limited to:

1) alkaloids and natural product drugs, including, microtubule inhibitors (e.g., Vincristine, Vinblastine, and Vindesine, and vinorelbine etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-161, and Teniposide [VM-261, etc.), and agents that target topoisomerase I (e.g., Camptothecin, topotecan (Hycamtin) and Irinotecan [CPT-11], rubitecan (Orathecin) etc.);

2) covalent DNA-binding agents [alkylating agents], including, nitrogen mustards (e.g., Mechlorethamine, chlormethine, Chlorambucil, Cyclophosphamide, estramustine (Emcyt, Estracit), ifosfamide, Ifosphamide, melphalan (Alkeran) etc.); alkyl sulfonates like Busulfan [Myleran], nitrosoureas (e.g., Carmustine or BCNU (bis-chloroethylnitrosourea), fotemustine Lomustine, and Semustine, streptozocin etc.), and other alkylating agents (e.g., Dacarbazine, procarbazine ethylenimine/methylmelamine, thriethylenemelamine (TEM), triethylene thiophosphoramide (thiotepa), hexamethylmelamine (HMM, altretamine), and Mitocycin, uramustine etc.) including Temozolomide (brand names Temodar and Temodal and Temcad), altretamine (also hexylen) and mitomycin;

3) noncovalent DNA-binding agents [antitumor antibiotics], including nucleic acid inhibitors (e.g., Dactinomycin [Actinomycin D1, etc.), anthracyclines (e.g., Daunorubicin [Daunomycin, and Cerubidine], Doxorubicin [Adrianycin], epirubicin (Ellence), and Idarubicin [Idamycin], valrubicin (Valstar) etc.), anthracenediones (e.g., anthracycline analogues, such as, [Mitoxantrone], etc.), bleomycins (Blenoxane), etc., amsacrine and plicamycin (Mithramycin), dactinomycin, mitomycin C:

4) antimetabolites, including, antifolates (e.g., Methotrexate, Folex, aminopterin, pemetrexed, raltitrexed and Mexate, trimetrexate etc.), purine antimetabolites (e.g., 6-Mercaptopurine [6-MP, Purinethol], cladribine, 6-Thioguanine [6-TG], clofarabine (Clolar, Evoltra), Azathioprine, Acyclovir, Fludarabine or fludarabine phosphate (Fludara) Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)], capecitabine Carmofur or HCFU (1-hexylcarbamoyl-5-fluorouracil), tegafur etc.), gemcitabine (Gemzar), and cytosine arabinosides (e.g., Cytarabine, or cytosine arabinoside, Cytosar [ara-C] and Fludarabine, 5-azacytidine, 2,2′-difluorodeoxycytidine etc.) and hydroxyurea (Hydrea and Droxia, hydroxycarbamide), plus lonidamine;

5) enzymes, including, L-asparaginase and derivatives such as pegaspargase (Oncaspar), and RNAse A;

7) hormones and antagonists, Examples of hormones and hormonal analogues believed to be useful in the treatment of neoplasms include, but are not limited to antiestrogens and selective estrogen receptor modulators (SERMs), such as tamoxifen, toremifene, raloxifene, iodoxyfene, droloxifene, 4-hydroxytamoxifen, trioxifene, keoxifene, onapristone; anti-androgens; such as enzalutamide (Xtandi®), flutamide, nilutamide, bicalutamide, leuprolide, and goserelin, and cyproterone acetate; adrenocorticosteroids such as prednisone and prednisolone; aminoglutethimide, finasteride and other aromatase inhibitors such as anastrozole, letrazole, vorazole, exemestane, formestanie, and fadrozole; Estrogen Receptor Downregulators (EROs) including Faslodex or fulvestrant, progestrins such as megestrol acetate; Sa-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

8) platinum compounds (e.g., Cisplatin and Carboplatin, oxaliplatin, Triplatin tetranitrate (rINN; also known as BBR3464), eptaplatin, lobaplatin, nedaplatin, or satraplatin etc.);

9) retinoids such as bexarotene (Targretin).

10) proteasome inhibitors such as bortezomib and carfilzomib (Kyprolis®).

11) anti-mitotics in addition to diterpenoids and vinca alkaloids include polo-like kinase (PLK) inhibitors, mitotic kinesin spindle protein (KSP) inhibitors including SB-743921 and MK-833 and CenpE inhibitors.

12) monoclonal antibodies, including cancer immunotherapy monoclonal antibodies and humanized monoclonal antibodies. For example:

12-a) cancer immunotherapy monoclonal antibodies include agents selected from the group consisting of Trastuzumab (Herceptin®), an example of an anti-erbB2 antibody inhibitor of growth factor function; cetuximab (Erbitux™, C225), an example of an anti-erbB 1 antibody inhibitor of growth factor function; bevacizumab (Avastin®), an example of a monoclonal antibody directed against VEGFR; rituximab, alemtuzumab, gemtuzumab, panitumumab, tositumomab, pertuzumab.

12-b) humanized monoclonal antibodies with therapeutic potential as chemotherapeutic agents in combination include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab (Perjeta®), pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab;

13) monoclonal antibodies conjugated with anticancer drugs, toxins, and/or radionuclides, etc. gemtuzumab ozogamicin (MYLOTARG), trastuzumab emtansine (T-DM1)/ado-trastuzumab emtansine (Kadcyla®);

14) biological response modifiers (e.g., interferons [e.g., IFN-.alpha., etc.] and interleukins [e.g., IL-2, etc.], denileukin diftitox (Ontak), G-CSF, GM-CSF: etc.);

15) adoptive immunotherapy; Immunotherapeutic regimens include ex-vivo and in-vivo approaches to increasing immunogenicity of patient tumor cells such as transfection with cytokines (eg. IL-2 or aldesleukin, IL-4, GMCFS), as well as IL-1, IL-3, IL-4, IL-5, IL-6, IL-7, IL-8, IL-9, IL-10, IL-11, IL-12, and active biological variants approaches to increase T-cell activity, approaches with transfected immune cells and approaches with antiidiotypic antibodies;

16) immunosuppressant selected from the group consisting of fingolimod, cyclosporine A, Azathioprine, dexamethasone, tacrolimus, sirolimus, pimecrolimus, mycophenolate salts, everolimus, basiliximab, daclizumab, anti-thymocyte globulin, anti-lymphocyte globulin, and tofacitinib. Agents capable of enhancing antitumor immune responses, such as CTLA4 (cytotoxic lymphocyte antigen 4) antibodies such as Ipilimumab (MDX-010 or MDX-101, Yervoy) and tremelimumab, and other agents capable of blocking CTLA4;

17) immune modulators, for use in conjunction with the compound as disclosed herein include staurosprine and macrocyclic analogs thereof, including UCN-01, CEP-701 and midostaurin; squalamine; DA-9601; alemtuzumab; interferons (e.g. IFN-a, IFN-b etc.); altretamine (Hexylen®); SU 101 or leflunomide; imidazoquinolines such as resiquimod, imiquimod, anti-PD-1 human monoclonal antibodies MDX-1106 (also known as BMS-936558), MK3475, CT-011, and AMP-224, anti-PD-L1 monoclonal antibodies such as MDX-1105, anti-OX40 monoclonal antibodies, and LAG3 fusion proteins such as IMP321g, anti-B7-H3 monoclonal antibodies such as MGA271, anti-B7-H4 monoclonal antibodies, and anti-TIM3 monoclonal antibodies;

18) hematopoietic growth factors;

19) agents that induce tumor cell differentiation (e.g., tretinoin (all trans retinoic acid) (brand names Aberela, Airol, Renova, Atralin, Retin-A, Avita, Retacnyl, Refissa, or Stieva-A));

20) gene therapy techniques; such as gene therapy vaccines, for example, ALLOVECTIN®, LEUVECTIN®, and V AXID®;

21) antisense therapy techniques;

22) tumor vaccines; include Avicine®; oregovomab (OvaRex®); Theratope® (STn-KLH); Melanoma Vaccines; G1-4000 series (GI-4014, G1-4015, and G1-4016), which are directed to five mutations in the Ras protein; GlioVax-1; MelaVax; Advexin® or INGN-201; Sig/E7/LAMP-1, encoding HPV-16 E7; MAGE-3 Vaccine or M3TK; HER-2VAX; ACTIVE, which stimulates T-cells specific for tumors; GM-CSF cancer vaccine; and Listeria onocytogenes-based vaccines;

23) therapies directed against tumor metastases (e.g., Batimistat, etc.);

24) inhibitors of angiogenesis. Receptor kinase angiogenesis inhibitors may also find use in the present invention. Inhibitors of angiogenesis related to VEGFR and TIE-2. Other inhibitors may be used in combination with the compounds of the invention. For example, anti-VEGF antibodies, which do not recognize VEGFR (the receptor tyrosine kinase), but bind to the ligand; small molecule inhibitors of integrin (alphav beta3) that inhibit angiogenesis; endostatin and angiostatin (non-RT) may also prove useful in combination with the compounds of the invention. One example of a VEGFR antibody is bevacizumab (Avastin®). Other anti-angiogenic compounds include acitretin, fenretinide, thalidomide, zoledronic acid, angiostatin, aplidine, cilengtide, combretastatin A-4, endostatin, halofuginone, rebimastat, removab, Lenalidomid (Revlimid), squalamine, Vitaxin, and pomalidomide (Pomalyst®);

25) signal transduction pathway inhibitors. Signal transduction pathway inhibitors are those inhibitors which block or inhibit a chemical process which evokes an intracellular change. As used herein these changes include, but are not limited to, cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositoi-3-0H kinases, myoinositol signaling, and Ras oncogenes. Signal transduction pathway inhibitors may be employed in combination with the compounds of the invention;

26) kinase inhibitors, including tyrosine kinases, serine/threonine kinases, kinases involved in the IGF-1 R signaling axis, PI3k/AKT/mTOR pathway inhibitors, and SH2/SH3 domain blockers. Examples of relevant kinases include:

26-a) tyrosine kinases. Several protein tyrosine kinases catalyze the phosphorylation of specific tyrosine residues in various proteins involved in the regulation of cell growth. Such protein tyrosine kinases can be broadly classified as receptor or non-receptor kinases. Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1 R, IR and IRR); epidermal growth factor family receptors (EGFR, ErbB2, and ErbB4); platelet derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-FMS), c-KIT, cMET, fibroblast growth factor receptors (FGFRs), hepatocyte growth factor receptors (HGFRs), Trk receptors (TrkA, TrkB, and TrkC), ephrin (Eph) receptors, the RET protooncogene, and Human Epidermal Growth Factor Receptor 2 (HER-2). Examples of small molecule inhibitors of epidermal growth factor receptors include but are not limited to gefitinib, lapatinib (Tykerb®), erlotinib (Tarceva®), afatinib (Gilotrif®, Tomtovok®, and Tovok®), and lmatinib (Gleevec®) is one example of a PDGFR inhibitor. Examples of VEGFR inhibitors include pazopanib (Votrient™), Vandetanib (ZD6474), AZD2171, vatalanib (PTK-787), Axitinib (AG013736; Inlyta®), dovitinib (CHIR-258), cabozantinib (Cometriq®), sunitinib, and sorafenib. Protein Kinase C (PKC) inhibitors, such as ruboxistaurin, AEB071 (Sotrastaurin) LY-317615 and perifosine. Examples of small molecule inhibitors of multiple tyrosine kinases include but are not limited to bosutinib (Bosulif®) and. Other kinase inhibitors include but are not limited to BIBF-1120, dasatinib (sprycel), pelitinib, nilotinib, and lestaurtinib (CEP-701). Tyrosine kinases that are not transmembrane growth factor receptor kinases are termed non-receptor, or intracellular tyrosine kinases. Inhibitors of non-receptor tyrosine kinases are sometimes referred to as “anti-metastatic agents” and are useful in the present invention. Targets or potential targets of anti-metastatic agents, include, but are not limited to, c-Src, Lck, Fyn, Yes, Jak, Abl kinase (c-Abl and Bcr-Abl), FAK (focal adhesion kinase) and Bruton's tyrosine kinase (BTK). Examples of small molecule inhibitors of Bcr-Abl include but are not limited to ponatinib (Iclusig®). Non-receptor kinases and agents, which inhibit non-receptor tyrosine kinase function, are described in Sinha, S. and Corey, S. J., J. Hematother. Stem Cell Res. (1999) δ 465-80; and Bolen, J. B. and Brugge, J. S., Annu. Rev. of Immunol. (1997) 15 371-404;

26-b) serine/threonine kinases. Inhibitors of serine/threonine kinases may also be used in combination with the compounds of the invention in any of the compositions and methods described above. Examples of serine/threonine kinase inhibitors that may also be used in combination with a compound of the present invention include, but are not limited to, polo-like kinase inhibitors (Pik family e.g., Plk1, Plk2, and Plk3), which play critical roles in regulating processes in the cell cycle including the entry into and the exit from mitosis; MAP kinase cascade blockers, which include other Ras/Raf kinase inhibitors, mitogen or extracellular regulated kinases (MEKs), and extracellular regulated kinases (ERKs); Aurora kinase inhibitors (including inhibitors of Aurora A and Aurora B); protein kinase C (PKC) family member blockers, including inhibitors of PKC subtypes (alpha, beta, gamma, epsilon, mu, lambda, iota, zeta); inhibitors of kappa-B (1 kB) kinase family (IKK-alpha, IKK-beta); PKB/Akt kinase family inhibitors; and inhibitors of TGF-beta receptor kinases. Examples of Plk inhibitors are described in PCT Publication No. W004/014899 and W007/03036;

26-c) kinases involved in the IGF-1 R signaling axis. Inhibitors of kinases involved in the IGF-1 R signaling axis may also be useful in combination with the compounds of the present invention. Such inhibitors include but are not limited to inhibitors of JNK1/2/3, PI3K, AKT and MEK, and 14.3.3 signaling inhibitors;

26-d) PI3k/AKT/mTOR pathway inhibitors, including GDC-0941, XL-147, GSK690693 and temsirolimus, SF-1126 (PI3K inhibitor), BEZ-235 (PI3K inhibitor);

26-e) SH2/SH3 domain blockers. SH2/SH3 domain blockers are agents that disrupt SH2 or SH3 domain binding in a variety of enzymes or adaptor proteins including, but not limited to, PI3-K p85 subunit, Src family kinases, adaptor molecules (She, Crk, Nck, Grb2) and Ras-GAP. Examples of Src inhibitors include, but are not limited to, dasatinib and BMS-354825 (J. Med. Chern. (2004) 4 7 6658-6661);

27) inhibitors of Ras oncogenes. Inhibitors of Ras oncogene may also be useful in combination with the compounds of the present invention. Such inhibitors include, but are not limited to, inhibitors of farnesyltransferase, geranyl-geranyl transferase, and CAAX proteases as well as anti-sense oligonucleotides, ribozymes and immunotherapy. Such inhibitors have been shown to block Ras activation in cells containing mutant Ras, thereby acting as antiproliferative agents.

28) Raf/MEK/ERK pathway modulators. The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability. Small molecule inhibitors of B-Raf are being developed for anticancer therapy. Examples of small molecule inhibitors of B-Raf include but are not limited to dabrafenib (Tafinlar®). Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example GSK-2118436, RAF-265, vemurafenib (Zelboraf, PLX-4032), PLX3603 and XL-281. Examples of small molecule inhibitors of MEK include but are not limited to trametinib (Mekinist®), Other MEK inhibitors include ARRY-886 (AZD6244);

29) Cell cycle signaling inhibitors, including inhibitors of cyclin dependent kinases (CDKs) are also useful in combination with the compounds of the invention in the compositions and methods described above. Examples of cyclin dependent kinases, including CDK2, CDK4, and CDK6 and inhibitors for the same are described in, for instance, Rosania G. R. et al., Exp. Opin. Ther. Patents (2000) 10 215-230;

30) Inhibitors of phosphatidyl inositoi-3-0H kinase family members including blockers of P13-kinase, ATM, DNA-PK, and Ku may also be useful in combination with the present invention;

31) Antagonists of smoothened receptor (SMO) may also be useful in combination with the present invention. Examples of antagonists of smoothened receptor include but are not limited to vismodegib (Erivedge®);

32) Inhibitors of protein translation may also be useful in combination with the present invention. Examples of inhibitors of protein translation include but are not limited to omacetaxine mepesuccinate (Synribo®); and

33) anti-cancer agents with other mechanisms of action including miltefosine (Impavido and Miltex), masoprocol, mitoguazone, alitretinoin, mitotane, arsenic trioxide, celecoxib, and anagrelide.

Compounds disclosed herein may also be employed in conjunction with anti-emetic agents to treat nausea or emesis, including acute, delayed, late-phase, and anticipatory emesis, which may result from the use of a compound as disclosed herein, alone or with radiation therapy. For the prevention or treatment of emesis, a compound as disclosed herein may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the instant compounds.

A compound as disclosed herein may also be administered with an agent useful in the treatment of anemia. Such an anemia treatment agent is, for example, a continuous eythropoiesis receptor activator (such as epoetin alfa).

A compound as disclosed herein may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound as disclosed herein may also be useful for treating or preventing cancer in combination with siRNA therapeutics.

A compound as disclosed herein may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); Axitinib (Inlyta®); azacitidine (Vidaza®); bevacuzimab (Avastin®); bexarotene capsules (Targretin®); bexarotene gel (Targretin®); bicalutamide (Casodex®), bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); flutamide (Eulexin®), fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); ipilimumab (Yervoy®), irinotecan (Camptosar®); lapatinib (TYKERB®), lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); panitumumab (VECTIBIX®), pamidronate (Aredia®); Pazopanib (Votrient®), pegademase (Adagen (Pegademase Bovine)®); pegaspargase (Oncaspar®); Pegfilgrastim (Neulasta®); pemetrexed disodium (Alimta®); pentostatin (Nipent®); pertuzumab (OMNITARG®, 2C4), pipobroman (Vercyte®); plicamycin, mithramycin (Mithracin®); porfimer sodium (Photofrin®); procarbazine (Matulane®); quinacrine (Atabrine®); Rapamycin (Sirolimus, RAPAMUNE®), Rasburicase (Elitek®); Rituximab (Rituxan®); rubitecan (Orathecin), ruxolitinib (Jakafi®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/1-131 tositumomab (Bexxar®); Trastuzumab (Herceptin®); tretinoin, ATRA (Vesanoid®); Uracil Mustard (Uracil Mustard Capsules®); valrubicin (Valstar®); vandetanib (ZACTIMA®), vemurafenib (Zelboraf®), vinblastine (Velban®); vincristine (Oncovin®); vinorelbine (Navelbine®); vorinostat (Zolinza®); zoledronate (Zometa®), nilotinib (Tasigna®); and dasatinib (Sprycel®). ARRY-886 (Mek inhibitor, AZD6244), SF-1126 (PI3K inhibitor), BEZ-235 (PI3K inhibitor), XL-147 (PI3K inhibitor), PTK787/ZK 222584, crizotinib (Xalkori®), and vemurafenib (Zelboraf®).

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating disorders and symptoms relating cancer in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of disorders and symptoms relating to cancer.

The compounds, compositions, and methods disclosed herein are useful for the treatment of disease. In certain embodiments, the diseases is one of dysregulated cellular proliferation, including cancer. The cancer may be hormone-dependent or hormone-resistant, such as in the case of breast cancers. In certain embodiments, the cancer is a solid tumor. In other embodiments, the cancer is a lymphoma or leukemia. In certain embodiments, the cancer is and a drug resistant phenotype of a cancer disclosed herein or known in the art. Tumor invasion, tumor growth, tumor metastasis, and angiogenesis may also be treated using the compositions and methods disclosed herein. Precancerous neoplasias are also treated using the compositions and methods disclosed herein.

Cancers to be treated by the methods disclosed herein include colon cancer, breast cancer, ovarian cancer, lung cancer and prostrate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL)) and lymphomas including lymphocytic, granulocytic and monocytic. Additional types of cancers which may be treated using the compounds and methods of the invention include, but are not limited to, adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In certain embodiments, the compositions and methods disclosed herein are useful for preventing or reducing tumor invasion and tumor metastasis.

In certain embodiments, the compositions and methods disclosed herein are useful for preventing or reducing angiogenesis and disorders related to angiogenesis. Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### LIST OF ABBREVIATIONS

CHCl₃=chloroform; i-PrOH=isopropanol; H₂O=water; DCM=dichloromethane; Na₂SO₄=sodium sulfate; MgSO₄=magnesium sulfate; EtOAc=ethyl acetate; EtOH=ethanol; Et₂O=diethyl ether; THF=tetrahydrofuran; NaOH=sodium hydroxide; NMP=N-Methyl-2-pyrrolidone; MeOH=methanol; CDCl₃=deuterated chloroform; HCl=hydrochloric acid; MeCN=acetonitrile; Cs₂CO₃=cesium carbonate; DMF=N,N-dimethylformamide; CD₃OD=deuterated methanol; DMSO-d₆=deuterated dimethyl sulfoxide; DMSO=dimethyl sulfoxide; TFA=trifluoroacetic acid; AcOH=acetic acid; HBr=hydrobromic acid; HCOOH=formic acid; K₂CO₃=potassium carbonate; DBU=1,8-diazabicyclo[5.4.0]undec-7-ene; NaHCO₃=sodium hydrogen carbonate; KCN=potassium cyanide; TEA=Et₃N=triethylamine; DMAP=4-dimethylaminopyridine; NH₂OH.HCl=hydroxylammonium chloride; DIEA=N,N-diisopropylethylamine; LiOH=lithium hydroxide; NH₄HCO₃=ammonium hydrogen carbonate; NH₄OH=ammonium hydroxide; K₃PO₄=potassium phosphate tribasic; NaOtBu=sodium t-butoxide; CuBr₂=copper (II) bromide; CuCl₂=copper (II) chloride; CuCN(LiCl)₂=Copper(I) cyanide di(lithium chloride) complex; EDC.HCl=1-Ethyl-3-(3-dimethylaminopropyl)carbodiimide hydrochloride; HOBT=1-hydroxybenzotriazole; PyBop=(Benzotriazol-1-yloxy)tripyrrolidinophosphonium hexafluorophosphate; CDI=1,1′-Carbonyldiimidazole; LiCl=lithium chloride; NaI=sodium iodide; NaBr=sodium bromide; N₂=nitrogen; Ar=argon; MnO₂=manganese dioxide; HATU=2-(1H-7-Azabenzotriazol-1-yl)-1,1,3,3-tetramethyl uronium hexafluorophosphate methanaminium; BH₃-THF=borane tetrahydrofuran complex solution; POCl₃=phosphorus oxychloride; Ac₂O=acetic anhydride; NH₂NH₂.H₂O=hydrazine hydrate; NaBH₄=sodium borohydride; LAH=lithium aluminiumhydride; NaBH₃CN=sodium cyanoborohydride; n-BuLi=n-butyllithium; CH₃I=methyl iodide; CS₂=carbon disulfide; AIBN=azobisisobutyronitrile; KF=potassium fluoride; Bu₃SnH=tributyltin hydride; RuPhos=2-Dicyclohexylphosphino-2′,6′-diisopropoxybiphenyl; XPhos=2-Dicyclohexylphosphino-2′,4′,6′-triisopropylbiphenyl; and Pd₂(dba)₃=Tris(dibenzylideneacetone)dipalladium(0); Pd(Ph₃)₄=tetrakis(triphenylphosphine)palladium(0); NBS=N-bromosuccinimide; NCS=N-chlorosuccinimide; CBr₄=tetrabromomethane; DEAD=diethyl azodicarboxylate; OsO₄=osmium tetraoxide; DIBAL-H=di-iso-butyl aluminium hydride; t-BuOH=tert-butanol; Py=pyridine; NaOMe=sodium methoxide; prep-HPLC=preparative high-performance liquid chromatography.

### General Methods for Preparing Compounds

The following schemes can be used to practice the present invention. Additional structural groups, including but not limited to those defined elsewhere in the specification and not shown in the compounds described in the schemes can incorporated to give various compounds disclosed herein, or intermediate compounds which can, after further manipulation using techniques known in the art, be converted to compounds of the present invention. For example, in ceratin embodiments the A, B, D, and E rings in the structures described in the schemes can be substituted with various groups as defined herein.

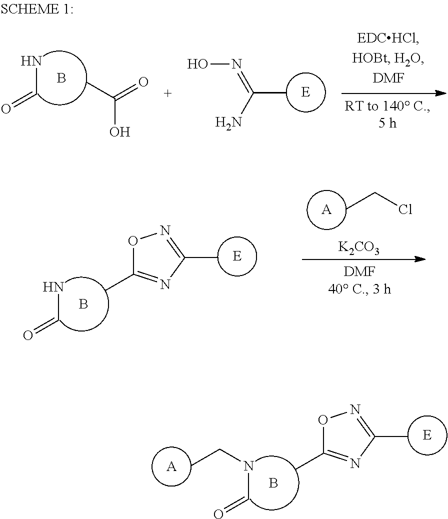

One route for preparation of compounds of the present invention is depicted in Scheme 1. A substituted functionalized heteroaromatic carboxylic acid is coupled with a hydroxyamidine in a solvent such as DMF, using reagents such as EDC and HOBT, warming the reaction to 140° C. for several hours. An alternative method for coupling the carboxylic acids and the hydroxylamidine is utilizing CDI in DCM, after formation of the initial adduct exchanging the solvent for DMF, and heating the reaction at 140° C., resulting in formation of the desired oxadiazole. The resulting heterocyclic derivative can then be alkylated with a suitable substituted benzylic halide or heteroaromatic methyl halide, or synthetic equivalent, for example using a base such as K₂CO₃ or CS₂CO₃, adding NaI to facilitate the alkylation if necessary. This transformation is typically conducted in a polar solvent like DMF at 25-60° C. to yield a mixture of O— and N— alkylated products, that can be separated using techniques known to those trained in the art like column chromatography of preparative HPLC. Separation of the regioisomers yields compounds of this invention.

Compounds of the present invention can be further manipulated using synthetic transformations known to those trained in the art to yield alternative compounds. For instance, as is depicted in Scheme 2, compounds bearing a protected amine can be deprotected, for example a Cbz-protecting group can be removed utilizing HBr in AcOH/H₂O. In turn the free amine can undergo a reductive amination reaction to yield higher substituted amines. For instance, by reacting the amine with an aldehyde in the presence of a reducing agent such as NaBH₃(CN) in a alcoholic solvent.

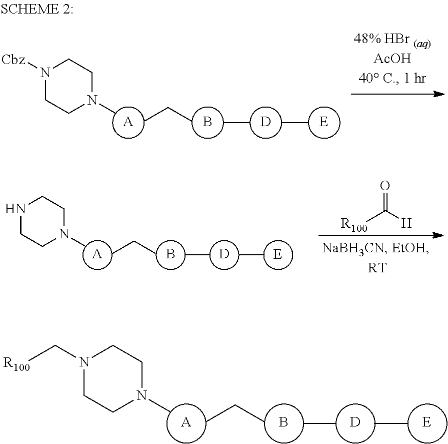

Alternatively, compounds of the present invention containing a primary or a secondary amine can be further manipulated by reaction with a carboxylic acid in the presence of coupling agents like EDCI or HATU, a carboxylic acid chloride, or a sulfonyl chloride in the presence of a base such as triethylamine in a suitable solvent, for example DCM, as depicted in Scheme 3.

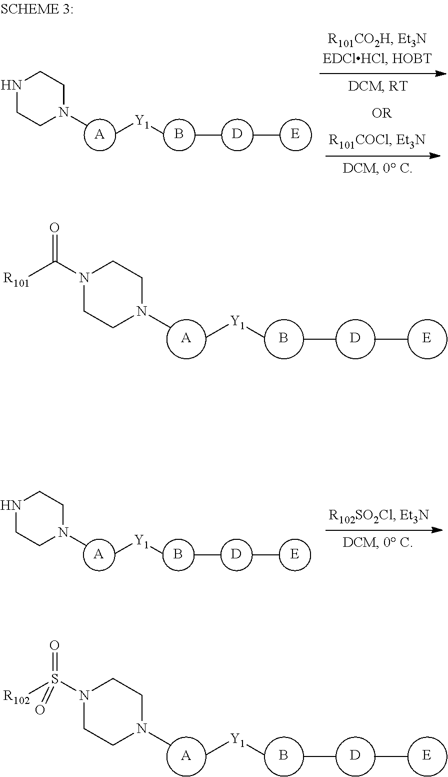

Another route for preparation of compounds of the present invention is depicted in Scheme 4, whereby a suitably elaborated heterocyclic carboxylic acid is coupled to an aniline using reagents such as PyBOP and DIPEA, in a polar solvent such as DMF. The resulting heterocyclic amide can then be alkylated with a suitable substituted benzylic halide or heteroaromatic methyl halide, or synthetic equivalent. For example using a base such as K₂CO₃ or CS₂CO₃ in a polar solvent like DMF at 25-40° C., to yield a mixture of O— and N— alkylated products that can be separated using techniques known to those trained in the art like column chromatography or preparative HPLC. Separation of the regioisomers yields compounds of the invention.

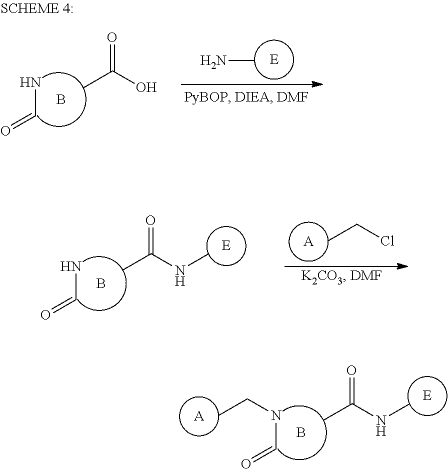

Compounds of the invention bearing 2-halopyridines, such as 2-chloropyridine, or heterocycles bearing a similarly reactive halogen substituents, for instance 2-chloropyrimidines or 2-chlorothiazoles, can be displaced with a variety of nucleophiles, such as primary and secondary amines [Scheme 5]. For example, by refluxing an excess of the amine at 120-150° C. overnight, using a solvent such as DMSO if required.

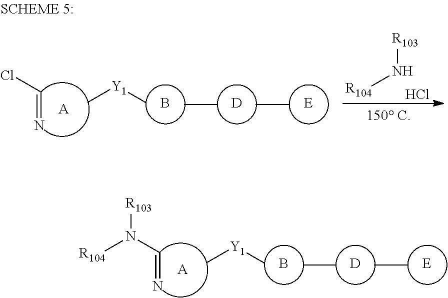

Alternatively, these heterocyclic halides or aromatic halides can be cross coupled with amines using palladium catalysis, using methods known to those trained in the art [Scheme 6]. For example, aromatic bromides can be coupled using a catalyst system comprising of dicyclohexyl(2′,6′-diisopropoxy-[1,1′-biphenyl]-2-yl)phosphine and chloro-(2-dicyclohexylphosphino-2′,6′-diisopropoxy-1,1′-biphenyl)[2-(2′-amino-1,1′-biphenyl)]palladium(II) in the presence of a base such as NaOtBu in THF at 65° C. for 2 h. Alternatively, a catalyst system comprising of XPhos, Pd₂(dba)₃ and N′,N′-dimethylethane-1,2-diamine in the presence of a base such as Cs₂CO₃ can be used for the transformation in a solvent such as dioxane, the reaction being conducted by microwave irradiation at 110-120° C. for 1 h. In addition, this transformation can be conducted using a catalytic system comprising of X-Phos and Pd₂(dba)₃ in the presence of a base such as Cs₂CO₃ in a solvent such as toluene, by heating at 140° C. up to 18 h.

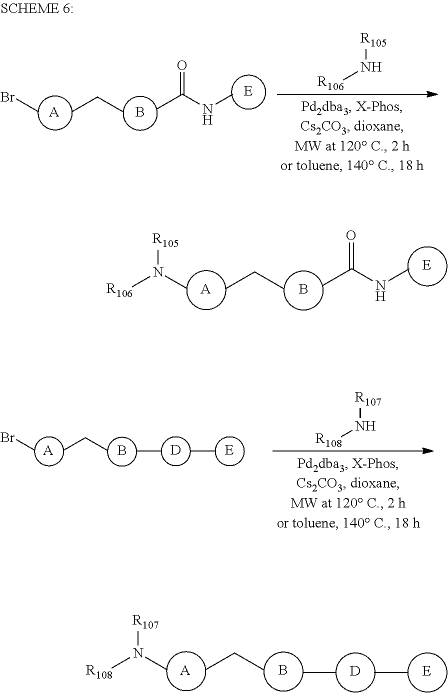

Compounds of the invention bearing a phenol moiety can be further manipulated by reaction with a variety of alkylating reagents such as alkyl-, benzyl- or allyl-halides, in the presence of a base such as K₂CO₃ or CsCO₃ in a suitable solvent like DMF, occasionally heating the reaction if necessary [Scheme 7a]. In addition, if such transformations are carried out with compounds of the invention bearing 2-hydroxypyridines, the products of N-alkylation can be obtained as well as the products of O-alkylation. An additional way to functionalize the compounds of this invention bearing a phenol moiety is described in Scheme 7b, and consists in reacting a suitable alcohol in the presence of an azodicarboxylate reagent, like diethyl azodicarboxylate (DEAD) and a phosphine, for example Ph₃P, according to the methodology described by Mistunobu et al. (Synthesis 1981, 1-28) or any modification thereof known to those trained in the art.

### Scheme 7

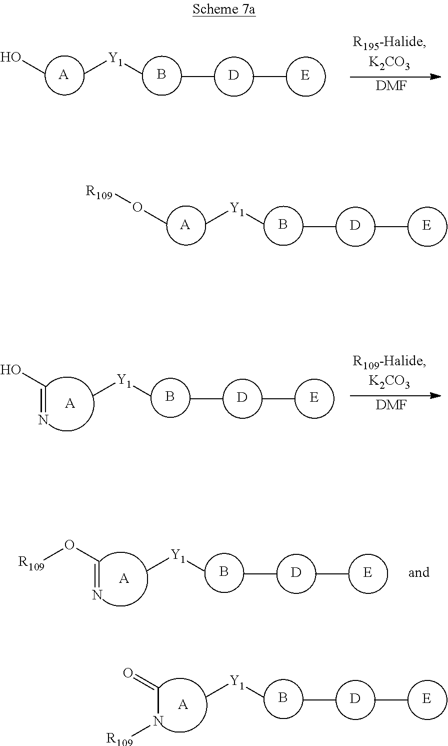

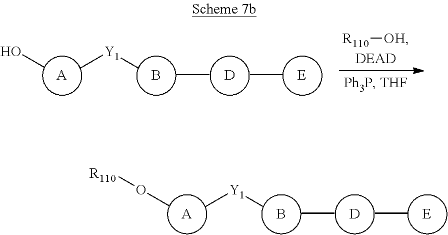

Furthermore, these heterocyclic halides or aromatic halides can be cross coupled with stannanes or boronates using palladium catalysis, applying methods known to those trained in the art [Scheme 8]. For example, aromatic bromides can be coupled with alkenyl stannanes using a catalyst system comprising of tetrakis(triphenylphosphine)palladium(0) in the presence of a base such as K₂CO₃ in a solvent such as dioxane, conducting the reaction thermally at 110° C. for 12 h.

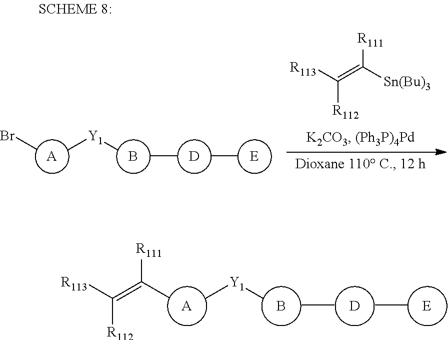

An additional route to prepare the compounds of this invention is described in Scheme 9. Compounds bearing an alkenyl group can be further manipulated by oxidative transformations. For example, they can undergo di-hydroxylation by applying methods known to those trained in the art, including using a catalytic amount of OsO₄ in the presence of N-methylmorpholine-oxide in a suitable solvent such as tert-butanol. Alternatively, the double bond can be converted into the corresponding epoxide by a suitable oxidizing reagent, for example 3-chloro-benzoperoxy acid, and the epoxide species could in turn be further functionalized by reaction with a suitable nucleophilic reagent such as an amine [Scheme 9].

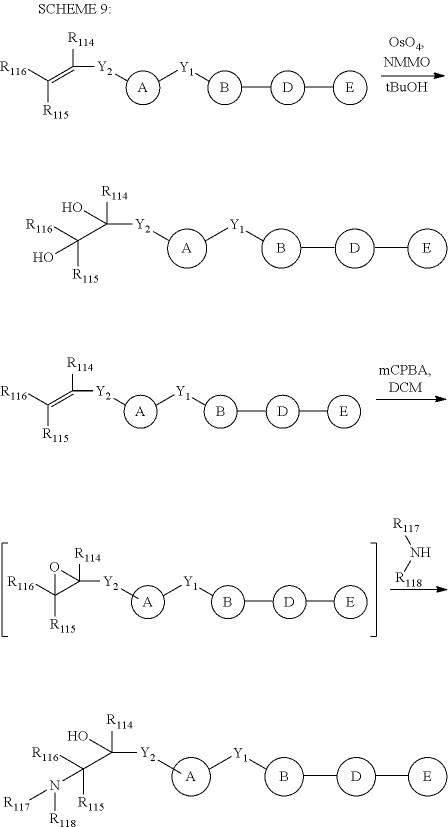

Alternatively, compounds of this invention can be prepared starting from a suitably substituted pyrimidine derivative which can be cross coupled with substituted benzylic and heterocyclicmethyl zinc derivatives using palladium catalysis in a solvent such as THF/toluene at elevated temperatures [Scheme 10]. Displacement of a halide group on the pyrimidine can then be accomplished using a cyanide source like KCN in a polar solvent such as DMSO with heating, for instance microwave irradiation. Palladium catalyzed cross coupling of a suitable aromatic bromides to a amine can be coupled using a catalyst system such as Ru-Phos and Ru-Phos precatalysts in the presence of a base such as Cs₂CO₃ in DMF at 95° C. in a microwave. Conversion of the nitrile to an oxadiazole can be accomplished using a hydroxyamidine in the presence of zinc chloride and pTSA in a solvent such as DMF at 100° C. to yield compounds of this invention.

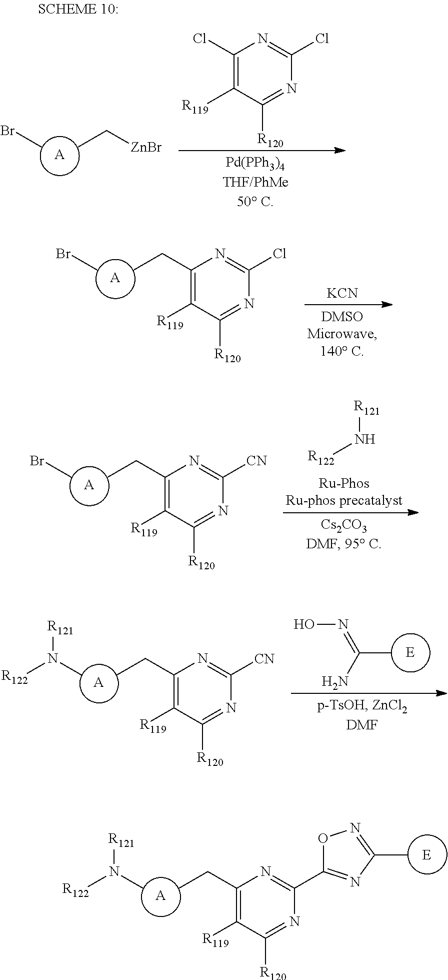

In addition, compounds of this invention containing a functionalized pyrazine ring can be prepared as described in Scheme 11. Reaction of acetylacetone with a suitably functionalized benzyl halide in the presence of a base such as LiOH, and in a polar solvent like DMF, results in the formation of alpha-benzyl substituted acetylacetone derivatives. The latter can in turn be converted to functionalized alpha-keto methyl ketones by treatment with nitrosobenzene in the presence of a base such as NaH, in a solvent like THF at low temperature. Condensation with a 2,3-diaminopropanoate followed by treatment with a suitable oxidizing agent like DDQ completes the synthesis of a highly functionalized pyrazine intermediate. The latter can then be converted to the compounds described in this invention for example by treatment with a suitable amideoxime, as previously described herein. Alternatively, the pyrazine intermediate can be further manipulated to form a variety of D rings according to the methods described in this invention. Examples of D ring that can be obtained include the isomeric oxadiazoles, as well as oxazoles, thiazoles and isoxazoles. Substituents on the A and E rings can also be further manipulated according to the methods described herein or to other chemical transformations known to those skilled in the art.

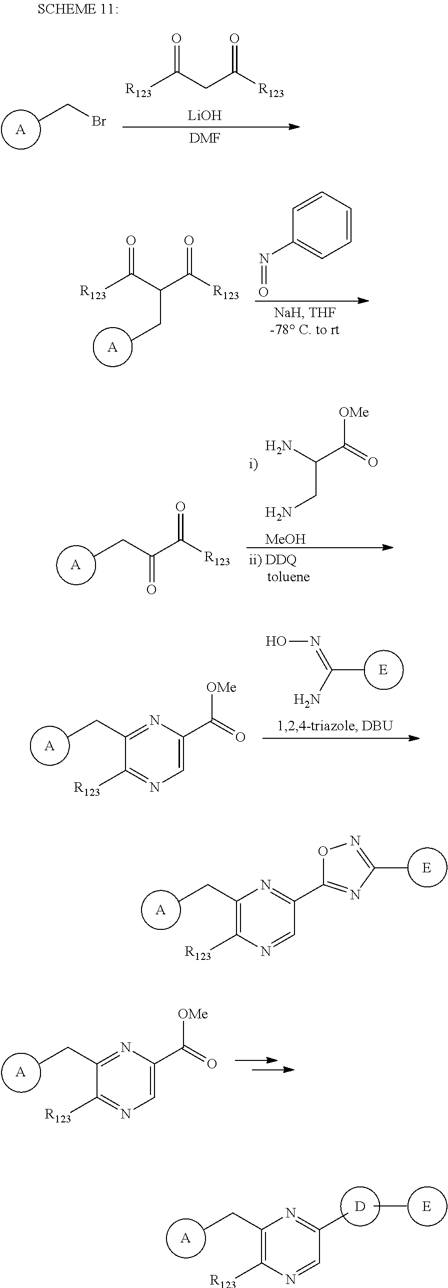

Another route for preparation of compounds of the present invention is depicted in Scheme 12, whereby a suitably elaborated heterocyclic derivative is alkylated with a benzylic or heterocyclic methyl halide or tosylate bearing a ester functional group, using a base such as Cs₂CO₃ in a solvent such as DMF. The ester can be transformed into amides of this invention using a variety method known to those trained in the art. For instance, the ester can be treated with an excess of the amine, in the presence of KCN, in a solvent such as THF under microwave irradiation to yield the desired compounds. Alternatively, the ester can be hydrolyzed to the corresponding acid using a base such as LiOH in a solvent mixture of MeOH, THF and water. In turn, the carboxylic acid can be coupled with amines using a variety of coupling reagents such as PyBOP in the presence of a tertiary amine base in solvents like DCM/THF. Another alternative is utilizing a mixture of the amine, 1,2,4-triazole, and DBU at elevated temperature.

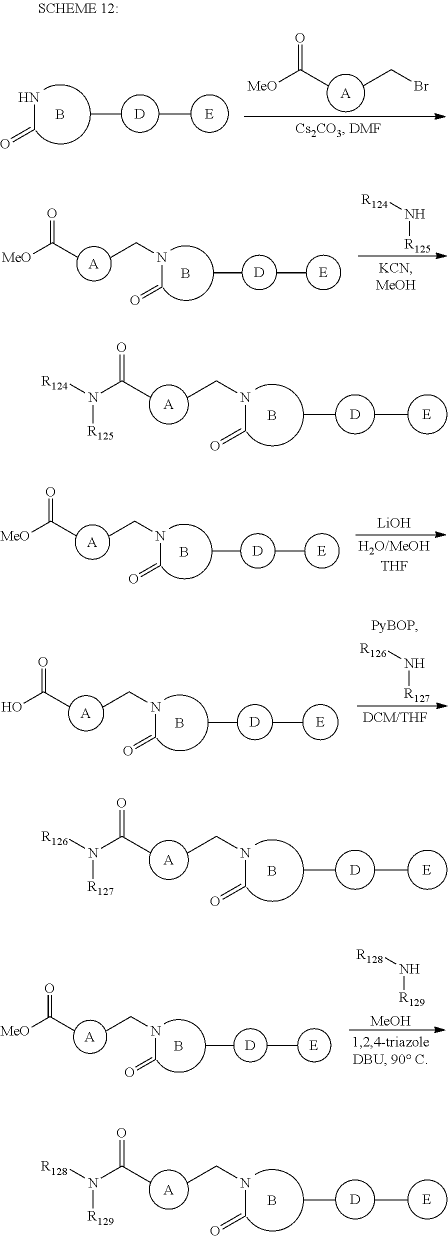

Additionally, compounds described in this invention bearing a carboxylic acid group can be further manipulated as described in Scheme 13. Treatment with a Grignard reagent, for example MeMgBr in a solvent such as THF results in the formation of the corresponding tertiary alcohol. An alternative way to prepare the required alcohol analogs is by treating the compounds bearing a substituted acetate group with an alkylating agent, such as MeI, in the presence of a strong base like NaH, in a solvent such as THF. Reduction of the carboxylic ester with a suitable agent such as LAH in THF gives then the 2-substituted alcohol compounds.

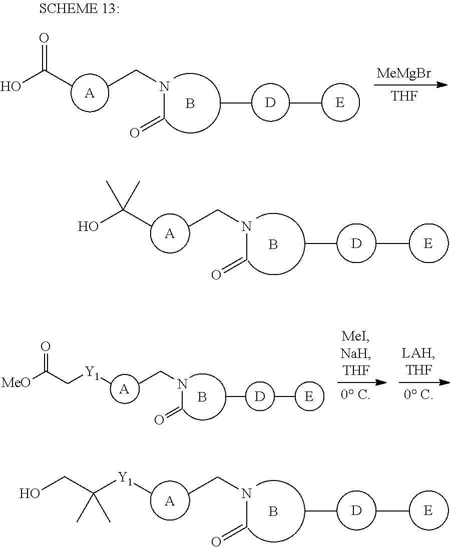

An additional route to prepare the compounds described in this invention which contain a 1,2,4-oxadiazole ring is depicted in Scheme 14. A suitably substituted heterocyclic nitrile can be alkylated with a functionalized benzyl halide in the presence of a base like Cs₂CO₃, and further progressed to the corresponding amidoxime by reaction with hydroxylamine hydrochloride in a solvent such as EtOH and in the presence of a base like TEA. The amideoxime can then be further reacted with a carboxylic acid of choice in the presence of a coupling reagent such as CDI to give the targeted 1,2,4-oxadiazole analog. Reactive functional groups that might be present on the A and/or the E ring (including halides, phenol, carboxylic acid, nitrile, amine, sulfide etc.) might then be further manipulated according to the transformations described herein, or to other general functional group transformations known to those skilled in the art.

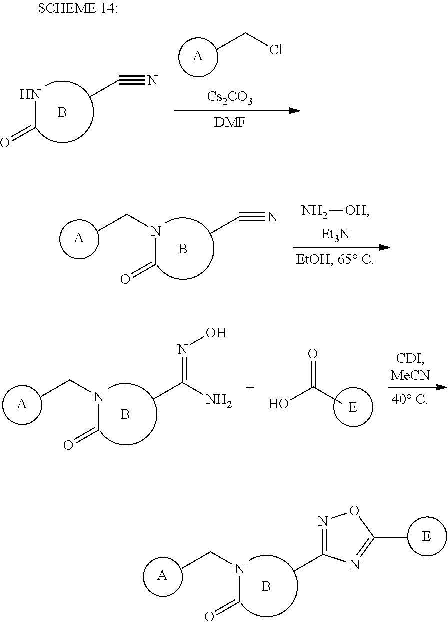

Alternatively, compounds of this invention can be prepared starting from a suitably substituted heterocyclic carboxylic acid which can be coupled with an acyl hydrazide to yield the intermediate, for example by using HATU and DIPEA in DMF [Scheme 15]. The resulting acyclic material can be cyclized to the corresponding 1,3,4-oxadiazole for instance by using Burgess' reagent in THF with microwave irradiation at 60° C. Alkylation with substituted benzylic and heterocyclic methyl halides and tosylates yields isomeric derivatives, for example using a base such as Cs₂CO₃ and NaI in a solvent like DMF at elevated temperature. These derivatives can be separated using chromatographic methods, and the desired regioisomer can be isolated. In turn they can be further functionalized using the transformations described elsewhere to yield other examples of this invention.

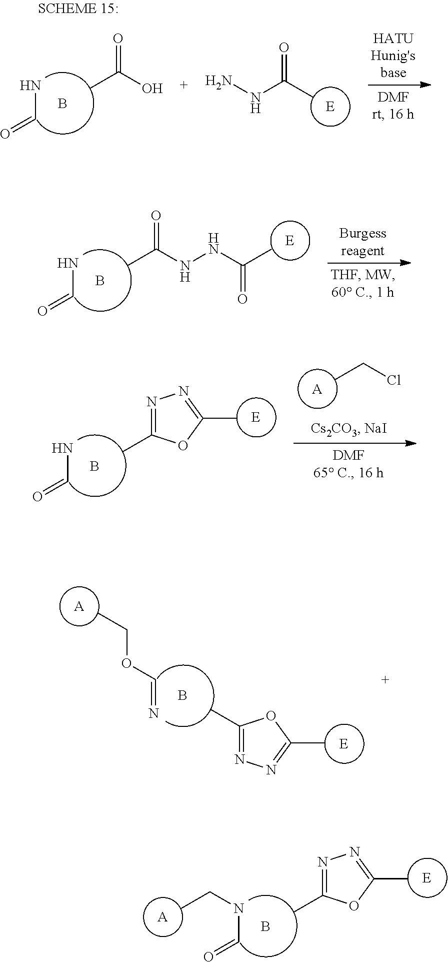

Compounds of this invention bearing a 2,5-disubstituted oxazole ring can be prepared starting from a suitable heterocyclic carboxylic acid [Scheme 16a]. Coupling with an alpha-amino ketone in the presence of a reagent such as CDI in a solvent like DCM, followed by condensation in the presence of a strong acid like H₂SO₄, results in the formation of an oxazole-containing tricyclic intermediate. The latter can in turn be alkylated to install substituent A. Both substituents A and E can be further manipulated as described in this invention or according to other chemical transformations known to those skilled in the art. Alternatively, compounds of this invention containing a 2,4-disubstituted oxazole ring can be prepared starting from a suitably substituted amino acid, as described in Scheme 16b. Treatment with a reducing agent like LAH in a solvent such as THF, with heating if necessary, results in the formation of the corresponding 2-amino-alcohol, which can in turn be progressed the 4,5-dihydro-oxazolidine intermediate by condensation in the presence of a suitable reagent like SOCl₂. Treatment with an oxidative reagent such as DDQ in a solvent like toluene results in the formation of an oxazole-containing tricyclic intermediate, which can then be alkylated with a suitable benzyl halide. Substituents A and E can be further manipulated as described in this invention or according to other chemical transformations known to those skilled in the art.

### Scheme 16

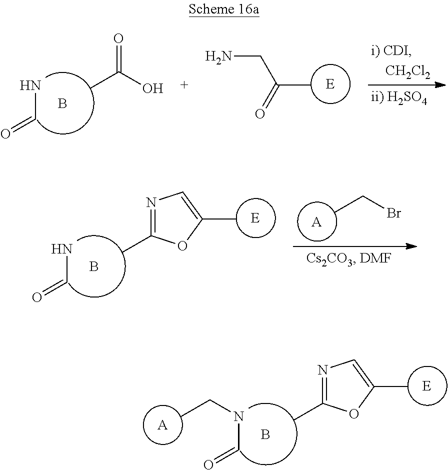

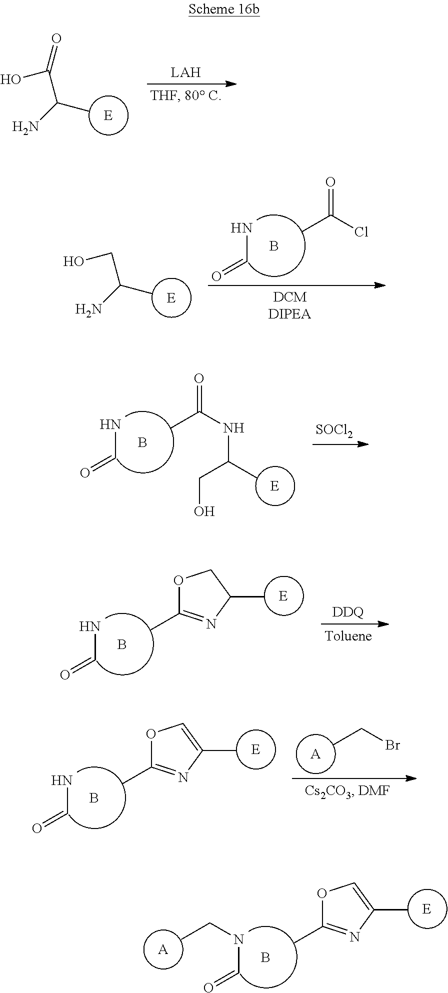

In addition, compounds of this invention bearing a substituted isoxazole can be prepared according to the route described in Scheme 17. A suitably substituted heterocyclic methyl ketone can be further functionalized by alkylation with a benzyl halide in the presence of a base like Cs₂CO₃, and then condensed with a suitable methyl ester in the presence of a base such as NaH in a solvent like THF. The 1,3-diketo-compound thus obtained can then be treated with hydroxylamine in a solvent like EtOH to give a mixture of the regioisomeric oxazole products. The latter can be separated by techniques known to those skilled in the art, such as preparative HPLC and SiO₂ gel chromatography. Rings A and E can be further manipulated according to the methods described herein and to other chemical transformations known to those skilled in the art.

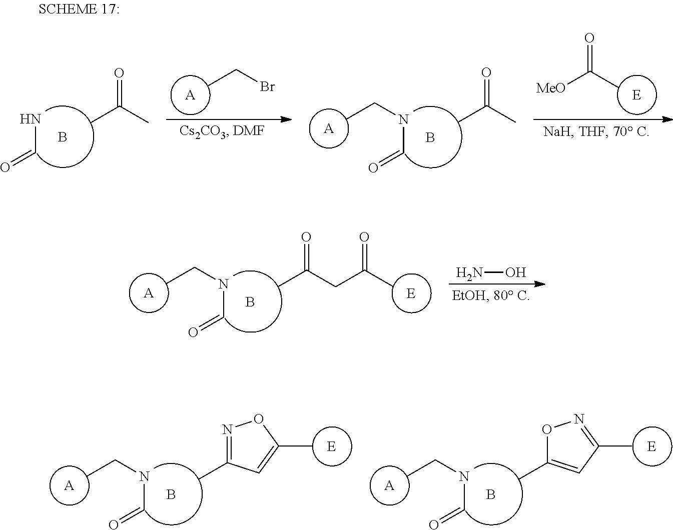

Alternatively, the compounds of this invention containing an isoxazole group can be obtained in a regioselective manner according to the routes described in Scheme 18a and Scheme 18b. A suitably functionalized methylketone can be condensed in a solvent like pyridine with an O-protected hydroxylamine, such as O-(4-methoxybenzyl)hydroxylamine [Scheme 18a]. Reaction with a suitably functionalized carboxaldehyde in the presence of a strong base, like n-BuLi, in a solvent like THF at low temperature, affords the corresponding hydroxyl addition product. The latter can in turn be oxidized with an agent such as Dess Martin periodinane in a solvent such as DCM. Upon deprotection of the hydroxylamine moiety, for example with an acid such as TFA, the resulting ketone can be cyclized into the required isoxazole product, which is obtained as a single regioisomer. The other isoxazole regioisomer can be accessed starting from an different set of methyl ketone and aldehyde building blocks, and using very similar transformations to those just described above, as described in Scheme 18b. In both cases, rings A and E can then be further functionalized employing the methods described herein or other chemical transformations known to those skilled in the art.

### Scheme 18

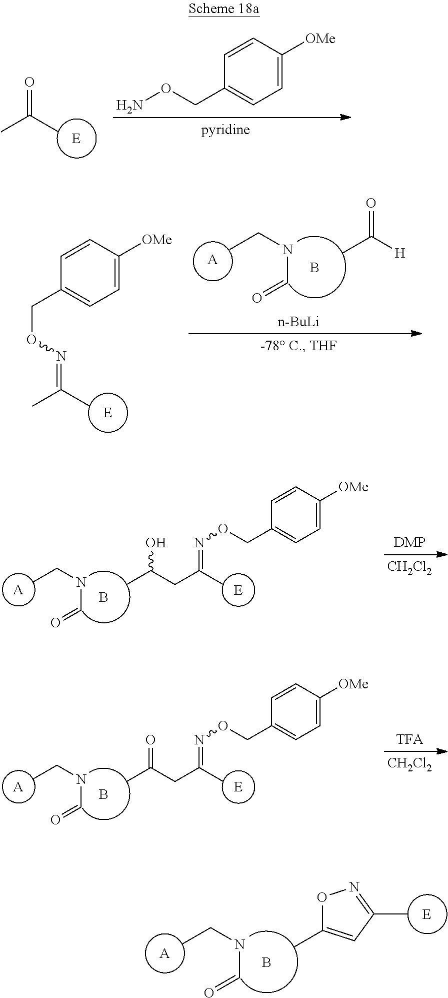

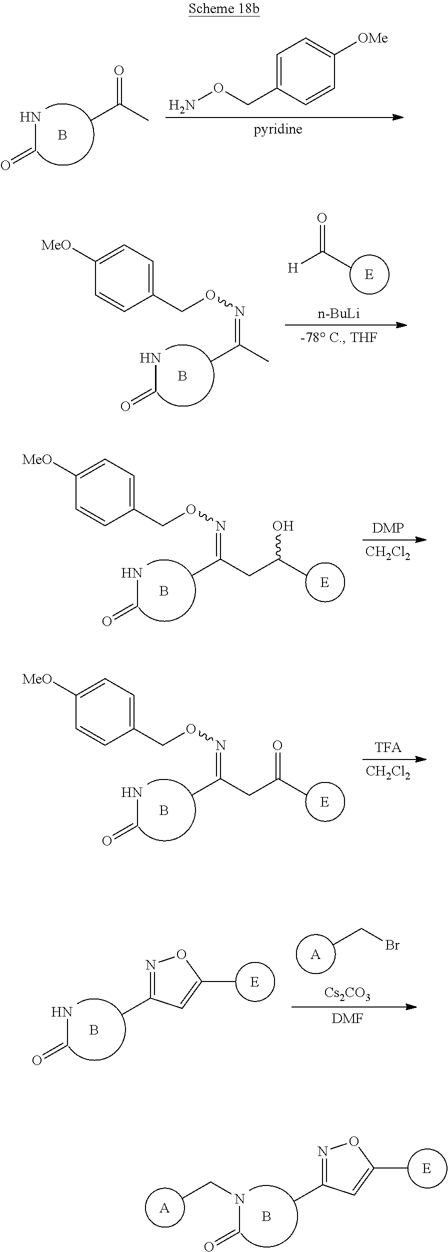

The invention is further illustrated by the following examples, which may be made my methods known in the art and/or as shown below. Additionally, these compounds may be commercially available.

### EXAMPLE 1

### 2-(2-(piperazin-1-yl)pyridin-4-yl)methyl)-6-(3-(4-(trifluoromethoxy)phenyl)-1,2,4-oxadiazol-5-yl)pyridazin-3(2H)-one

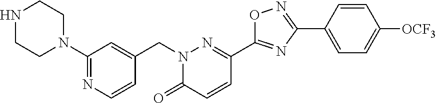 [SMILES: O=c1ccc(-c2nc(-c3ccc(OC(F)(F)F)cc3)no2)nn1Cc1ccnc(N2CCNCC2)c1]

### Step 1

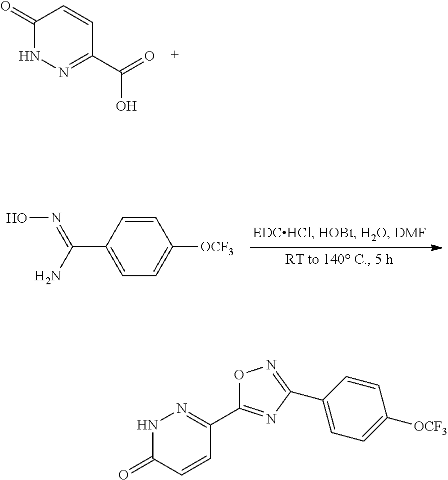 [SMILES: O=C(O)c1ccc(=O)[nH]n1 ; N/C(=N\O)c1ccc(OC(F)(F)F)cc1 ; O=c1ccc(-c2nc(-c3ccc(OC(F)(F)F)cc3)no2)n[nH]1]

### 6-(3-(4-Trifluoromethoxy)phenyl)-1,2,4-oxadiazol-5-yl)pyridazin-3(2H)-one

To a solution of 6-oxo-1,6-dihydropyridazine-3-carboxylic acid (1.00 g, 7.14 mmol) and EDC.HCl (1.9 g, 10.71 mmol) in DMF (20 mL), HOBT (546 mg, 3.57 mmol) and (Z)—N′-hydroxy-4-(trifluoromethoxy)benzimidamide (1.7 g, 7.86 mmol) were added. The reaction mixture was stirred at 140° C. for 5 h, then cooled to RT, treated with H₂O (100 mL), and extracted with EtOAc (100 mL). The organic layer was washed with H₂O (3×20 mL) and brine (3×20 mL), dried over Na₂SO₄, filtered, and concentrated to afford 6-(3-(4-(trifluoromethoxy)phenyl)-1,2,4-oxadiazol-5-yl)pyridazin-3(2H)-one as a yellow solid (1.1 g, 47%). MS (ES+) C₁₃H₇F₃N₄O₃ requires: 324. found: 325 [M+H]⁺.

### Step 2

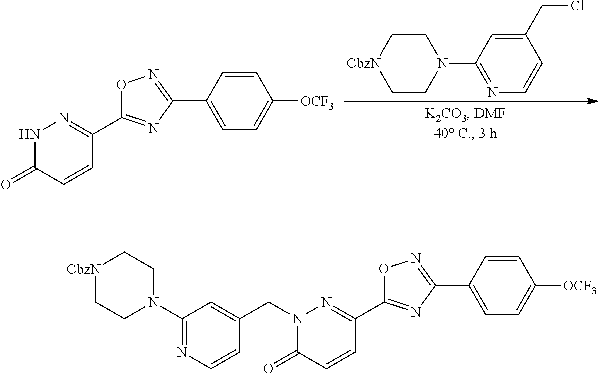 [SMILES: O=c1ccc(-c2nc(-c3ccc(OC(F)(F)F)cc3)no2)n[nH]1 ; O=C(OCc1ccccc1)N1CCN(c2cc(CCl)ccn2)CC1 ; O=C(OCc1ccccc1)N1CCN(c2cc(Cn3nc(-c4nc(-c5ccc(OC(F)(F)F)cc5)no4)ccc3=O)ccn2)CC1]

### Benzyl 4-(4-((6-oxo-3-(3-(4-(trifluoromethoxy)phenyl)-1,2,4-oxadiazol-5-yl)pyridazin-1(6H)-yl)methyl)pyridin-2-yl)piperazine-1-carboxylate

To a solution of 6-(3-(4-(trifluoromethoxy)phenyl)-1,2,4-oxadiazol-5-yl)pyridazin-3(2H)-one (400 mg, 1.23 mmol) in DMF (10 mL), benzyl 4-(4-(chloromethyl)pyridine-2-yl)piperazine-1-carboxylate (290 mg, 1.48 mmol) and K₂CO₃ (340 mg, 2.46 mmol) were added. The reaction mixture was stirred at 40° C. for 3 h, then cooled, treated with H₂O (50 mL), and extracted with EtOAc (50 mL). The organic layer was washed with H₂O (3×20 mL) and brine (3×20 mL), dried over Na₂SO₄, filtered, and concentrated to afford benzyl 4-(4-((6-oxo-3-(3-(4-(trifluoromethoxy)phenyl)-1,2,4-oxadiazol-5-yl)pyridazin-1 (6H)-yl)methyl)pyridin-2-yl)piperazine-1-carboxylate as a yellow solid (460 mg, 58%). MS (ES+) C₃₁H₂₆F₃N₇O₅ requires: 633. found: 634 [M+H]⁺.

### Step 3

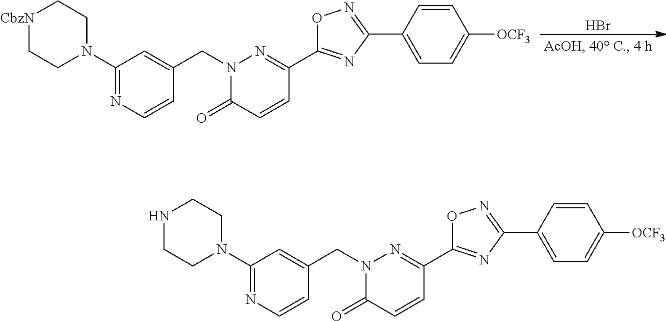 [SMILES: O=C(OCc1ccccc1)N1CCN(c2cc(Cn3nc(-c4nc(-c5ccc(OC(F)(F)F)cc5)no4)ccc3=O)ccn2)CC1 ; Br ; O=c1ccc(-c2nc(-c3ccc(OC(F)(F)F)cc3)no2)nn1Cc1ccnc(N2CCNCC2)c1]

### 2-(2-(piperazin-1-yl)pyridin-4-yl)methyl)-6-(3-(4-(trifluoromethoxy)phenyl)-1,2,4-oxadiazol-5-yl)pyridazin-3(2H)-one

To a solution of benzyl 4-(4-((6-oxo-3-(3-(4-(trifluoromethoxy)phenyl)-1,2,4-oxadiazol-5-yl)pyridazin-1(6H)-yl)methyl)pyridin-2-yl)piperazine-1-carboxylate (460 mg, 0.72 mmol) in AcOH (10 mL), HBr (5 mL, 48% in H₂O) was added. The reaction mixture was stirred at 40° C. for 4 h. After the removal of the volatiles under reduced pressure, the residue was treated neutralized with saturated aqueous NaHCO₃ and extracted with EtOAc (50 mL). The organic layer was washed with H₂O (3×20 mL) and brine (3×20 mL), then dried over Na₂SO₄, filtered, and concentrated to afford the crude product, which was purified by silica gel column chromatography (EtOAc:Petroleum ether=1:7) to afford 2-((2-(piperazin-1-yl)pyridin-4-yl)methyl)-6-(3-(4-(trifluoromethoxy)phenyl)-1,2,4-oxadiazol-5-yl)pyridazin-3(2H)-one as a yellow solid (300 mg, 83%). MS (ES+)C₂₃H₂₀F₃N₇O₃ requires: 499. found: 500 [M+H]⁺. ¹H-NMR (500 MHz, CD₃OD) δ ppm 8.20 (d, J=8.5 Hz, 2H), 8.19 (d, J=10.0 Hz, 1H), 8.11 (d, J=5.5 Hz, 1H), 7.45 (d, J=8.5 Hz, 2H), 7.20 (d, J=9.5 Hz, 1H), 6.95 (s, 1H), 6.74 (d, J=5.0 Hz, 1H), 5.42 (s, 2H), 3.80 (t, J=5.0 Hz, 4H), 3.34-3.32 (m, 1H), 3.28 (t, J=5.5 Hz, 4H).

### EXAMPLE 2

### 2-(2-(4-Methylpiperazin-1-yl)pyridin-4-yl)methyl)-6-(3-(4-(trifluoromethoxy)phenyl)-1,2,4-oxadiazol-5-yl)pyridazin-3(2H)-one

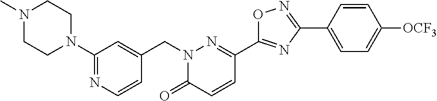 [SMILES: CN1CCN(c2cc(Cn3nc(-c4nc(-c5ccc(OC(F)(F)F)cc5)no4)ccc3=O)ccn2)CC1]

### Step 1

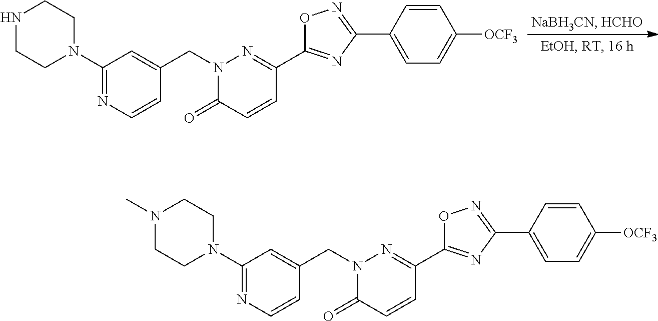

### 2-((2-(4-Methylpiperazin-1-yl)pyridin-4-yl)methyl)-6-(3-(4-(trifluoromethoxy)phenyl)-1,2,4-oxadiazol-5-yl)pyridazin-3(2H)-one

To a solution of 2-((2-(piperazin-1-yl)pyridin-4-yl)methyl)-6-(3-(4-(trifluoromethoxy)phenyl)-1,2,4-oxadiazol-5-yl)pyridazin-3(2H)-one (200 mg, 0.4 mmol) in EtOH (6 mL), NaBH₃CN (101 mg, 2 mmol) and formaldehyde (3 mL, 36.0 mmol, 38% in H₂O) were added. The reaction mixture was stirred at RT for 16 h, treated with H₂O (30 mL), and extracted with EtOAc (30 mL). The organic layer was washed with H₂O (3×20 mL) and brine (3×20 mL), dried over Na₂SO₄, filtered, and concentrated to afford the crude product, which was purified by pre-HPLC (Mobile phase: A=0.01% HCOOH/H₂O, B=MeCN; Gradient: B=60%-95% in 18 min; Column: XBridge C18, 5 um, 30 mm×150 mm) to afford 2-((2-(4-methylpiperazin-1-yl)pyridin-4-yl)methyl)-6-(3-(4-(trifluoromethoxy)phenyl)-1,2,4-oxadiazol-5-yl)pyridazin-3(2H)-one as a yellow solid (16 mg, 8%). MS (ES+) C₂₄H₂₂F₃N₇O₃ requires: 513. found: 514 [M+H]⁺; ¹H-NMR (500 MHz, CD₃OD) δ ppm 8.27 (d, J=8.5 Hz, 2H), 8.24 (d, J=10.0 Hz, 1H), 8.08 (d, J=5.5 Hz, 1H), 7.50 (d, J=8.5 Hz, 2H), 7.23 (d, J=9.5 Hz, 1H), 6.90 (s, 1H), 6.71 (d, J=5.5 Hz, 1H), 5.44 (s, 2H), 3.58 (t, J=4.5 Hz, 4H), 2.60 (t, J=5.0 Hz, 4H)), 2.38 (s, 3H).

### EXAMPLE 3

### 1-(2-(4-Methylpiperazin-1-yl)pyridin-4-yl)methyl)-6-oxo-N-(4-(trifluoromethoxy)phenyl)-1,6-dihydropyridazine-3-carboxamide bis trifluoroacetate salt

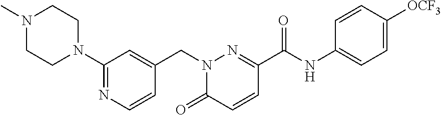 [SMILES: CN1CCN(c2cc(Cn3nc(C(=O)Nc4ccc(OC(F)(F)F)cc4)ccc3=O)ccn2)CC1]

### Step 1

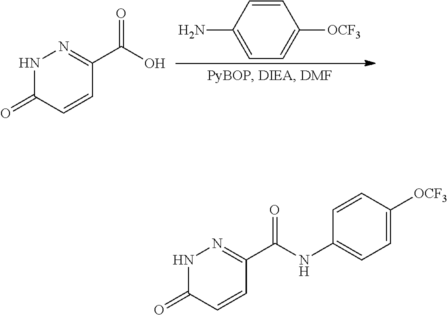 [SMILES: O=C(O)c1ccc(=O)[nH]n1 ; Nc1ccc(OC(F)(F)F)cc1 ; O=C(Nc1ccc(OC(F)(F)F)cc1)c1ccc(=O)[nH]n1]

### 6-oxo-N-(4-(trifluoromethoxy)phenyl)-1,6-dihydropyridazine-3-carboxamide

To a solution of 6-oxo-1,6-dihydropyridazine-3-carboxylic acid (500 mg, 3.16 mmol) in DMF (5 mL) at 25° C. 4-(trifluoromethoxy)aniline (616 mg, 3.48 mmol), DIEA (1.1 mL, 6.3 mmol) and PyBOP (1.643 g, 3.16 mmol) were added. The resulting mixture was stirred at 25° C. for 16 h, then diluted EtOAc and washed with brine. The organic layer was dried (Na₂SO₄), concentrated under reduced pressure and the residue was triturated with a 1/1 v/v mixture of Et₂O/DCM to give the title compound as a white solid (415 mg, 44%); MS (ES+) C₁₂H₈F₃N₃O₃ requires: 299. found: 300 [M+H]+.

### Step 2

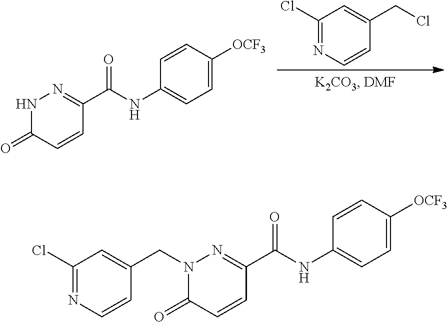 [SMILES: O=C(Nc1ccc(OC(F)(F)F)cc1)c1ccc(=O)[nH]n1 ; ClCc1ccnc(Cl)c1 ; O=C(Nc1ccc(OC(F)(F)F)cc1)c1ccc(=O)n(Cc2ccnc(Cl)c2)n1]

### 1-((2-Chloropyridin-4-yl)methyl)-6-oxo-N-(4-(trifluoromethoxy)phenyl)-1,6-dihydropyridazine-3-carboxamide

To a solution of 6-oxo-N-(4-(trifluoromethoxy)phenyl)-1,6-dihydropyridazine-3-carboxamide (200 mg, 0.669 mmol) in DMF (3 mL) 2-chloro-4-(chloromethyl)pyridine (140 mg, 0.702 mmol) and Cs₂CO₃ (436 mg, 1.338 mmol) were added and the resulting mixture was stirred at 25° C. for 16 h. The reaction mixture was then diluted with EtOAc and washed with brine. The organic layer was dried (Na₂SO₄) and concentrated under reduced pressure. The residue was purified by Biotage (50 g SNAP; 10 to 100% EtOAc in Hexane) to provide the title compound as a white solid (240 mg, 56%); MS (ES+) C₁₈H₁₂ClF₃N₄O₃ requires: 424. found: 425 [M+H]+.

### Step 3

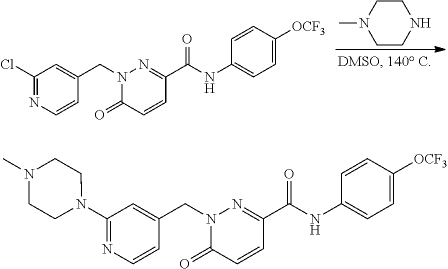

### 1-((2-(4-Methylpiperazin-1-yl)pyridin-4-yl)methyl)-6-oxo-N-(4-(trifluoromethoxy)phenyl)-1,6-dihydropyridazine-3-carboxamide bis trifluoroacetate salt

To a solution of 1-((2-chloropyridin-4-yl)methyl)-6-oxo-N-(4-(trifluoro methoxy)phenyl)-1,6-dihydropyridazine-3-carboxamide from Step 2 (26 mg, 0.06 mmol) in DMSO (0.5 mL) 1-methylpiperazine (61 mg, 0.61 mmol) was added and the resulting mixture was stirred at 140° C. for 16 h in a pressure vial. The reaction mixture was then diluted with DMSO (0.2 mL) and purified by Mass-triggered RP-HPLC to provide the title compound as a white solid after lyophilization (18 mg, 41%); MS (ES+)C₂₃H₂₃F₃N₆O₃ requires: 488. found: 489 [M+H]+; ¹H NMR (600 MHz, DMSO-d₆) δ10.46 (s, 1H), 9.65 (bs, 1H), 8.13 (d, J=5.2 Hz, 1H), 8.05 (d, J=9.7 Hz, 1H), 7.90 (d, J=9.1 Hz, 2H), 7.40 (d, J=8.7 Hz, 2H), 7.14 (d, J=9.8 Hz, 1H), 6.93 (s, 1H), 6.68 (d, J=5.2 Hz, 1H), 5.34 (s, 2H), 3.51-3.49 (m, 4H), 3.12-3.04 (m, 4H), 2.84 (s, 3H).

### EXAMPLE 4

### 1-(3-(4-Methylpiperazin-1-yl)benzyl)-6-oxo-N-(4-(trifluoromethoxy)phenyl)-1,6-dihydropyridazine-3-carboxamide trifluoroacetate salt

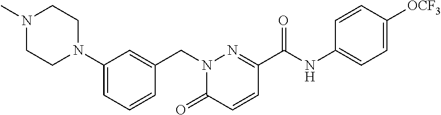 [SMILES: CN1CCN(c2cccc(Cn3nc(C(=O)Nc4ccc(OC(F)(F)F)cc4)ccc3=O)c2)CC1]

### Step 1

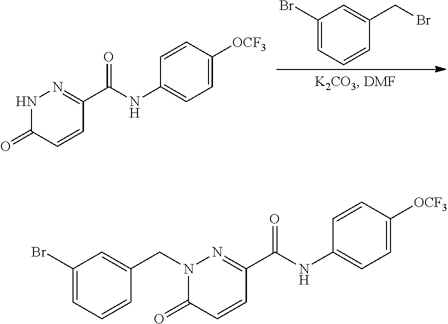 [SMILES: O=C(Nc1ccc(OC(F)(F)F)cc1)c1ccc(=O)[nH]n1 ; BrCc1cccc(Br)c1 ; O=C(Nc1ccc(OC(F)(F)F)cc1)c1ccc(=O)n(Cc2cccc(Br)c2)n1]

### 1-(3-Bromobenzyl)-6-oxo-N-(4-(trifluoromethoxy)phenyl)-1,6-dihydropyridazine-3-carboxamide

To a solution of 6-oxo-N-(4-(trifluoromethoxy)phenyl)-1,6-dihydropyridazine-3-carboxamide (200 mg, 0.669 mmol) in DMF (3 mL) 1-bromo-3-(bromomethyl)benzene (175 mg, 0.702 mmol) and Cs₂CO₃ (436 mg, 1.338 mmol) were added and the resulting mixture was stirred at 25° C. for 16 h. The reaction mixture was then diluted with EtOAc and washed with brine. The organic layer was dried (Na₂SO₄) and concentrated under reduced pressure. The residue was purified by Biotage (50 g SNAP; 10 to 100% EtOAc in Hexane) to provide the title compound as a pale yellow solid (153 mg, 49%); MS (ES+) C₁₉H₁₃BrF₃N₃O₃ requires: 467, 469 found: 468 [M+H]+, 470 [M+2+H]+(1:1).

### Step 2

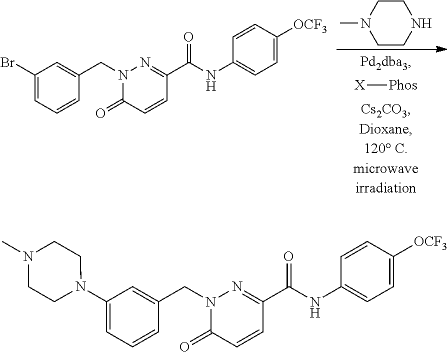

### 1-(3-(4-Methylpiperazin-1-yl)benzyl)-6-oxo-N-(4-(trifluoromethoxy)phenyl)-1,6-dihydropyridazine-3-carboxamide trifluoroacetate salt

To a solution of 1-(3-bromobenzyl)-6-oxo-N-(4-(trifluoromethoxy)phenyl)-1,6-dihydropyridazine-3-carboxamide from Step 1 (52 mg, 0.11 mmol) in 1,4-dioxane (1 mL, previously degassed under N₂ flux) 1-methylpiperazine (17 mg, 0.17 mmol), Pd₂(dba)₃ (4 mg, 0.004 mmol), XPhos (6 mg, 0.011 mmol) and Cs₂CO₃ (72 mg, 0.22 mmol) were added. The resulting mixture was stirred at 120° C. for 2 h under microwave irradiation. The reaction mixture was then filtered through a pad of Celite® and the filtrate was concentrated under reduced pressure. The residue was purified by Mass-triggered RP-HPLC to provide the title compound as a white solid after lyophilization (26 mg, 39%); MS (ES+) C₂₄H₂₄F₃N₅O₃ requires: 487. found: 488 [M+H]+; ¹H NMR (600 MHz, DMSO-d₆) δ 10.48 (s, 1H), 9.58 (bs, 1H), 7.98 (d, J=9.7 Hz, 1H), 7.91 (d, J=9.1 Hz, 2H), 7.41 (d, J=8.8 Hz, 2H), 7.24 (m, 1H), 7.11 (d, J=9.7 Hz, 2H), 6.96 (d, J=6.4 Hz, 1H), 6.87 (d, J=7.6 Hz, 1H), 5.34 (s, 2H), 3.84-3.82 (m, 4H), 2.96-2.92 (m, 4H), 2.85 (s, 3H).

### EXAMPLE 5

### 1-methyl-4-(4-((6-methyl-2-oxo-3-(3-(4-(trifluoromethoxy)phenyl)-1,2,4-oxadiazol-5-yl)pyridin-1(2H)-yl)methyl)pyridin-1-ium-2-yl)piperazin-1-ium bis-trifluoroacetate

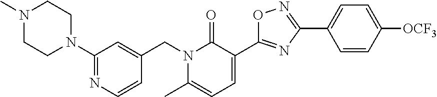 [SMILES: Cc1ccc(-c2nc(-c3ccc(OC(F)(F)F)cc3)no2)c(=O)n1Cc1ccnc(N2CCN(C)CC2)c1]

### Step 1

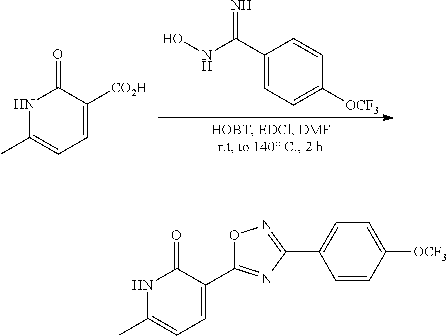 [SMILES: Cc1ccc(C(=O)O)c(=O)[nH]1 ; N=C(NO)c1ccc(OC(F)(F)F)cc1 ; Cc1ccc(-c2nc(-c3ccc(OC(F)(F)F)cc3)no2)c(=O)[nH]1]

### 6-Methyl-3-(3-(4-(trifluoromethoxy)phenyl)-1,2,4-oxadiazol-5-yl)pyridin-2(1H)-one

A reaction mixture of 6-methyl-2-oxo-1,2-dihydropyridine-3-carboxylic acid (200 mg, 1.3 mmol), EDC.HCl (200 mg, 1.3 mmol), and HOBT (200 mg, 1.6 mmol), and N-hydroxy-4-(trifluoromethoxy)benzimidamide (300 mg, 1.3 mmol) in DMF (13 mL) was stirred at RT for 8 h and then at 140° C. for an additional 2 h. The reaction mixture diluted with EtOAc, washed with H₂O, and concentrated to afford the crude product which was purified by silica gel chromatography (EtOAc/Hexane 20%-100% EtOAc) to afford 6-methyl-3-(3-(4-(trifluoromethoxy)phenyl)-1,2,4-oxadiazol-5-yl)pyridin-2(1H)-one as a white solid (114 mg, 26%). MS (ES+) C₁₅H₁₀F₃N₃O₃ requires: 337. found 338 [M+H]⁺.

### Step 2

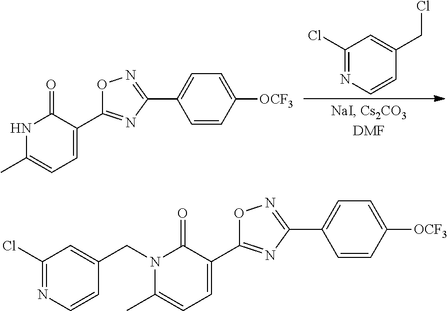 [SMILES: Cc1ccc(-c2nc(-c3ccc(OC(F)(F)F)cc3)no2)c(=O)[nH]1 ; ClCc1ccnc(Cl)c1 ; Cc1ccc(-c2nc(-c3ccc(OC(F)(F)F)cc3)no2)c(=O)n1Cc1ccnc(Cl)c1]

### 1-((2-Chloropyridin-4-yl)methyl)-6-methyl-3-(3-(4-(trifluoromethoxy)phenyl)-1,2,4-oxadiazol-5-yl)pyridin-2(1H)-one

A reaction mixture of 6-methyl-3-(3-(4-(trifluoromethoxy)phenyl)-1,2,4-oxadiazol-5-yl)pyridin-2(1H)-one (180 mg, 0.495 mmol), Cs₂CO₃ (518 mg, 1.6 mmol), NaI (300 mg, 2 mmol) and 2-chloro-4-(chloromethyl)pyridine (211 mg, 1.07 mmol) in DMF (3 mL) was stirred at 75° C. for 40 min. The reaction mixture was concentrated and purified by silica gel chromatography (EtOAc/Hexane 0%-100% EtOAc) to afford 1-((2-chloropyridin-4-yl)methyl)-6-methyl-3-(3-(4-(trifluoromethoxy)phenyl)-1,2,4-oxadiazol-5-yl)pyridin-2(1H)-one (91 mg, 37%). MS (ES+) C₂₁H₁₄ClF₃N₄O₃ requires: 462. found 463 [M+H]⁺

### Step 3

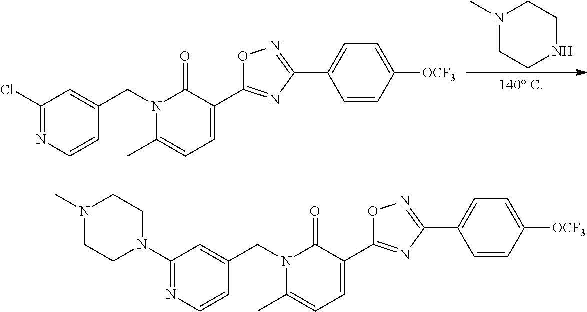

### 1-methyl-4-(4-((6-methyl-2-oxo-3-(3-(4-(trifluoromethoxy)phenyl)-1,2,4-oxadiazol-5-yl)pyridin-1(2H)-yl)methyl)pyridin-1-ium-2-yl)piperazin-1-ium bis-trifluoroacetate

1-((2-chloropyridin-4-yl)methyl)-6-methyl-3-(3-(4-(trifluoromethoxy)phenyl)-1,2,4-oxadiazol-5-yl)pyridin-2(1H)-one (14.3 mg, 0.03 mmol) in 1-methylpiperazine (0.5 mL) was heated at 140° C. for 4.5 h. The mixture was concentrated and purified by prep-HPLC (MeCN/H₂O 20%-60% MeCN, containing 0.1% TFA) to give the titled compound (4.3 mg, 19%). MS (ES+) C₂₆H₂₅F₃N₆O₃ requires: 526. found 527 [M+H]⁺; ¹H-NMR (600 MHz, CD₃OD) δ ppm 8.54 (d, J=7.2 Hz, 1H), 8.26 (d, J=7.8 Hz, 2H), 8.12 (d, J=5.4 Hz, 1H), 7.44 (d, J=8.4 Hz, 2H), 6.87 (s, 1H), 6.65 (d, J=5.4 Hz, 1H), 6.59 (d, J=8.4 Hz, 1H), 5.50 (s, 2H), 3.62-3.87 (m, 4H), 3.96-4.38 (m, 4H), 2.94 (s, 3H), 2.51 (s, 3H).

### EXAMPLE 6

### 1-Methyl-4-(3-((6-methyl-2-oxo-3-(3-(4-(trifluoromethoxy)phenyl)-1,2,4-oxadiazol-5-yl)pyridin-1(2H)-yl)methyl)phenyl)piperazin-1-ium trifluoroacetate

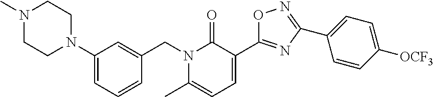 [SMILES: Cc1ccc(-c2nc(-c3ccc(OC(F)(F)F)cc3)no2)c(=O)n1Cc1cccc(N2CCN(C)CC2)c1]

### Step 1

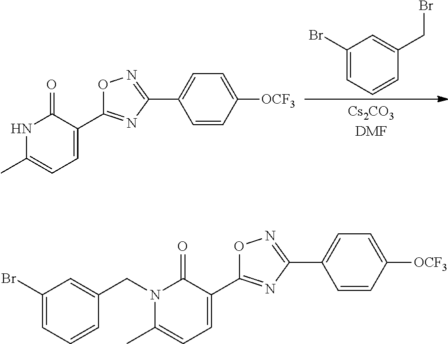 [SMILES: Cc1ccc(-c2nc(-c3ccc(OC(F)(F)F)cc3)no2)c(=O)[nH]1 ; BrCc1cccc(Br)c1 ; Cc1ccc(-c2nc(-c3ccc(OC(F)(F)F)cc3)no2)c(=O)n1Cc1cccc(Br)c1]

### 1-(3-Bromobenzyl)-6-methyl-3-(3-(4-(trifluoromethoxy)phenyl)-1,2,4-oxadiazol-5-yl)pyridin-2(1H)-one

6-Methyl-3-(3-(4-(trifluoromethoxy)phenyl)-1,2,4-oxadiazol-5-yl)pyridin-2(1H)-one (0.472 g, 1.4 mmol), 1-bromo-3-(bromomethyl)benzene (0.525 g, 2.100 mmol), and Cs₂CO₃ (0.685 g, 2.100 mmol) in DMF (10 ml) was stirred at 60° C. for 1 h. The reaction was diluted with EtOAc, washed with brine, and concentrated to give a crude product, which was purified by Biotage (0-100% EtOAc in Hexanes) to give the titled compound (210 mg, 30%). MS (ES+) C₂₂H₁₅BrF₃N₃O₃ requires: 505. found 506 [M+H]⁺

### Step 2

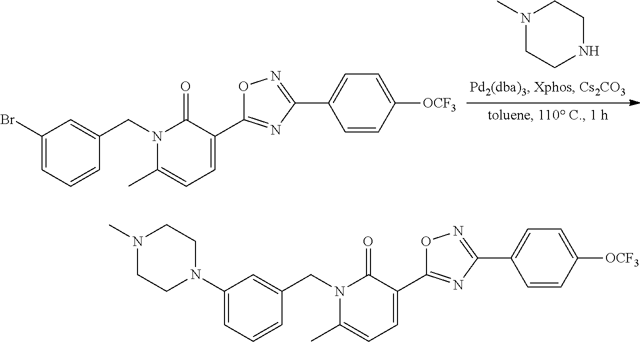

### 1-Methyl-4-(3-((6-methyl-2-oxo-3-(3-(4-(trifluoromethoxy)phenyl)-1,2,4-oxadiazol-5-yl)pyridin-1(2H)-yl)methyl)phenyl)piperazin-1-ium trifluoroacetate

A mixture of Pd₂(dba)₃ (5.1 mg, 5.57 μmol), XPhos (5.4 mg, 0.011 mmol), 1-(3-bromobenzyl)-6-methyl-3-(3-(4-(trifluoromethoxy)phenyl)-1,2,4-oxadiazol-5-yl)pyridin-2(1H)-one (23 mg, 0.045 mmol)1-methylpiperazine (4.55 mg, 0.045 mmol), Cs₂CO₃ (30.6 mg, 0.094 mmol) in toluene (0.6 ml) was degassed and filled with N₂. The reaction was heated to 110° C. for 2 h. The mixture was diluted with EtOAc, passed through a pad of celite, and concentrated to give a crude. Prep-HPLC purification furnished the titled compound (1.4 mg, 6%). MS (ES+) C₂₇H₂₆F₃N₅O₃ requires: 525. found 526 [M+H]⁺; ¹H-NMR (600 MHz, CD₃OD) δ ppm 8.49 (d, J=7.8 Hz, 1H), 8.26 (d, J=9.0 Hz, 2H), 7.45 (d, J=9.0 Hz, 2H), 7.28 (d, J=8.0 Hz, 1H), 6.96 (m, 1H), 6.92 (s, 1H), 6.76 (d, J=8.0 Hz, 1H), 6.50 (d, J=7.8 Hz, 1H), 5.51 (s, 2H), 3.38 (d, J=13.2 Hz, 2H), 3.56 (d, J=13.2 Hz, 2H), 3.23 (t, J=12.6 Hz, 2H), 3.01 (t, J=12.6 Hz, 2H), 2.94 (s, 3H), 2.51 (s, 3H).

### EXAMPLE 7

### 1-methyl-4-(4-((2-oxo-5-(3-(4-(trifluoromethoxy)phenyl)-1,2,4-oxadiazol-5-yl)pyridin-1(2H)-yl)methyl)pyridin-1-ium-2-yl)piperazin-1-ium bis-trifluoroacetate

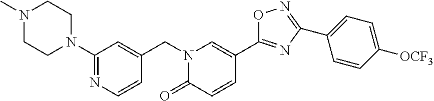 [SMILES: CN1CCN(c2cc(Cn3cc(-c4nc(-c5ccc(OC(F)(F)F)cc5)no4)ccc3=O)ccn2)CC1]

### Step 1

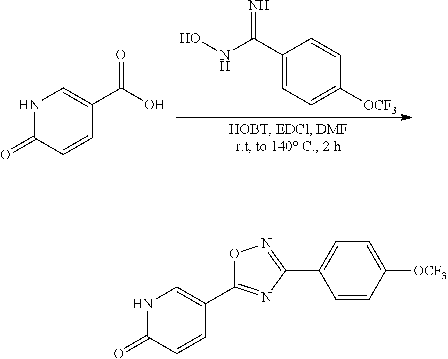 [SMILES: O=C(O)c1ccc(=O)[nH]c1 ; N=C(NO)c1ccc(OC(F)(F)F)cc1 ; O=c1ccc(-c2nc(-c3ccc(OC(F)(F)F)cc3)no2)c[nH]1]

### 5-(3-(4-(Trifluoromethoxy)phenyl)-1,2,4-oxadiazol-5-yl)pyridin-2(1H)-one

Synthesized in an analogous method to Example 5 Step 1 (120 mg, 30%): MS (ES+) C₁₄H₈F₃N₃O₃ requires: 323. found: 324 [M+H]⁺

### Step 2

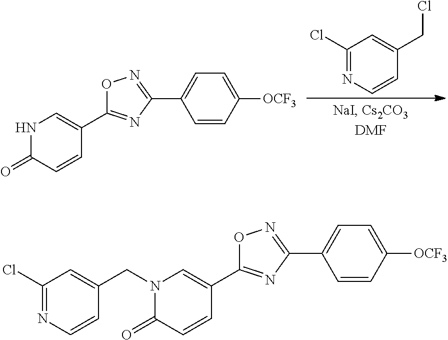 [SMILES: O=c1ccc(-c2nc(-c3ccc(OC(F)(F)F)cc3)no2)c[nH]1 ; ClCc1ccnc(Cl)c1 ; O=c1ccc(-c2nc(-c3ccc(OC(F)(F)F)cc3)no2)cn1Cc1ccnc(Cl)c1]

### 1-((2-Chloropyridin-4-yl)methyl)-5-(3-(4-(trifluoromethoxy)phenyl)-1,2,4-oxadiazol-5-yl)pyridin-2(1H)-one

Synthesized in an analogous method to Example 5 Step 2 (234 mg, 49%): MS (ES+) C₂₀H₁₂ClF₃N₄O₃ requires: 448. found: 449 [M+H]⁺.

### Step 3

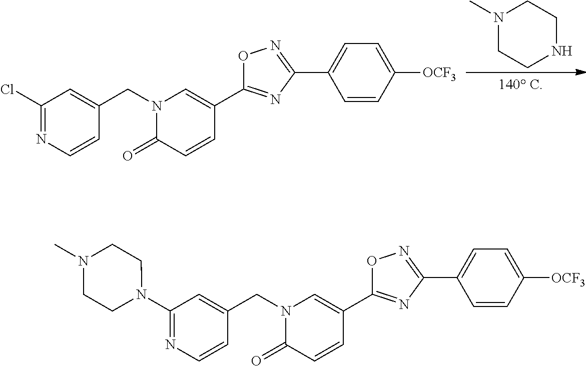

### 1-methyl-4-(4-((2-oxo-5-(3-(4-(trifluoromethoxy)phenyl)-1,2,4-oxadiazol-5-yl)pyridin-1(2H)-yl)methyl)pyridin-1-ium-2-yl)piperazin-1-ium bis-trifluoroacetate

Synthesized in an analogous method to Example 5 Step 3 (9.2 mg, 11%): MS (ES+) C₂₅H₂₃F₃N₆O₃ requires: 512. found: 513 [M+H]⁺; ¹H-NMR (600 MHz, CD₃OD) δ ppm 8.85 (s, 1H), 8.82 (m, 3H), 8.14 (d, J=5.4 Hz, 1H), 7.45 (d, J=8.4 Hz, 2H), 6.96 (s, 1H), 6.74 (m, 2H), 5.28 (s, 2H), 3.01-3.24 (m, 4H), 3.26-3.54 (m, 4H), 2.94 (s, 3H).

### EXAMPLE 8

### 1-methyl-4-(4-((6-methyl-2-oxo-3-((4-(trifluoromethoxy)phenyl)carbamoyl)pyridin-1(2H)-yl)methyl)pyridin-1-ium-2-yl)piperazin-1-ium bis-trifluoroacetate

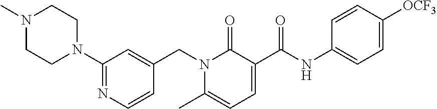 [SMILES: Cc1ccc(C(=O)Nc2ccc(OC(F)(F)F)cc2)c(=O)n1Cc1ccnc(N2CCN(C)CC2)c1]

### Step 1

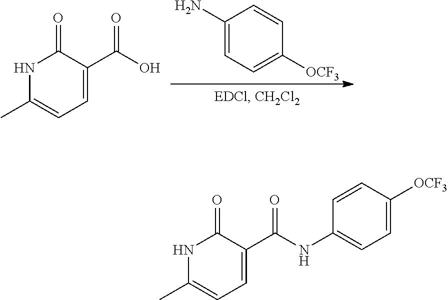 [SMILES: Cc1ccc(C(=O)O)c(=O)[nH]1 ; Nc1ccc(OC(F)(F)F)cc1 ; Cc1ccc(C(=O)Nc2ccc(OC(F)(F)F)cc2)c(=O)[nH]1]

### 6-Methyl-2-oxo-N-(4-(trifluoromethoxy)phenyl)-1,2-dihydropyridine-3-carboxamide

A reaction mixture of 6-methyl-2-oxo-1,2-dihydropyridine-3-carboxylic acid (300 mg, 2 mmol), EDC.HCl (380 mg, 2 mmol), 4-(trifluoromethoxy)aniline (350 mg, 2 mmol), TEA (0.3 mL, 2 mmol) and DMAP (50 mg, 0.4 mmol) in DCM (4 mL) was stirred at RT for 8 h. The reaction mixture was filtered to give the titled compound as a white solid (225 mg, 36%). MS (ES+) C₁₄H₁₁F₃N₂O₃ requires: 312. found: 313 [M+H]⁺

### Step 2

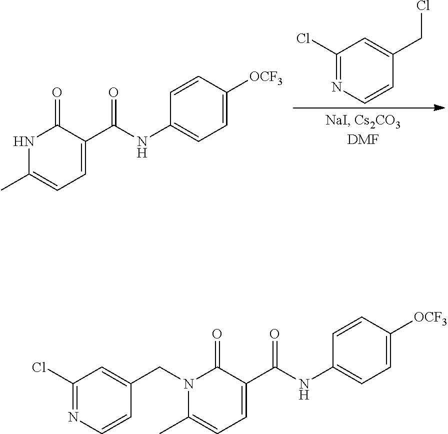 [SMILES: Cc1ccc(C(=O)Nc2ccc(OC(F)(F)F)cc2)c(=O)[nH]1 ; ClCc1ccnc(Cl)c1 ; Cc1ccc(C(=O)Nc2ccc(OC(F)(F)F)cc2)c(=O)n1Cc1ccnc(Cl)c1]

### 1-((2-chloropyridin-4-yl)methyl)-6-methyl-2-oxo-N-(4-(trifluoromethoxy)phenyl)-1,2-dihydropyridine-3-carboxamide

synthesized in an analogous method to Example 5 Step 2 (94 mg, 33%): MS (ES+) C₂₀H₁₅ClF₃N₃O₃ requires: 437. found: 438 [M+H]⁺.

### Step 3

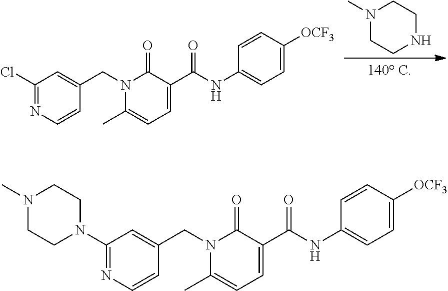

1-methyl-4-(4-((6-methyl-2-oxo-3-((4-(trifluoromethoxy)phenyl)carbamoyl)pyridin-1(2H)-yl)methyl)pyridin-1-ium-2-yl)piperazin-1-ium bis-trifluoroacetate

Synthesized in an analogous method to Example 5 Step 3 (7.6 mg, 15%). MS (ES+) C₂₅H₂₆F₃N₅O₃ requires: 501. found: 502 [M+H]⁺; ¹H-NMR (600 MHz, DMSO-d₆) δ ppm 12.11 (s, 1H), 9.71 (s, 1H), 8.47 (d, J=7.8 Hz, 1H), 8.10 (d, J=4.8 Hz, 1H), 7.81 (d, J=7.8 Hz, 2H), 7.35 (d, J=8.4 Hz, 2H), 6.78 (s, 1H), 6.66 (d, J=7.2 Hz, 1H), 6.41 (d, J=4.8 Hz, 1H), 5.41 (s, 2H), 4.35 (d, J=6.6 Hz, 2H), 3.49 (d, J=10.8 Hz, 2H), 3.08 (m, 4H), 2.84 (s, 3H), 2.44 (s, 3H).

### EXAMPLE 9

### 1-methyl-4-(4-((2-oxo-3-((4-(trifluoromethoxy)phenyl)carbamoyl)pyridin-1(2H)-yl)methyl)pyridin-1-ium-2-yl)piperazin-1-ium bis-trifluoroacetate

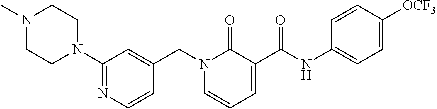 [SMILES: CN1CCN(c2cc(Cn3cccc(C(=O)Nc4ccc(OC(F)(F)F)cc4)c3=O)ccn2)CC1]

### Step 1

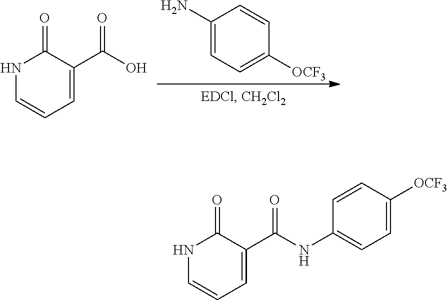 [SMILES: O=C(O)c1ccc[nH]c1=O ; Nc1ccc(OC(F)(F)F)cc1 ; O=C(Nc1ccc(OC(F)(F)F)cc1)c1ccc[nH]c1=O]

### 2-oxo-N-(4-(trifluoromethoxy)phenyl)-1,2-dihydropyridine-3-carboxamide

Synthesized in an analogous method to Example 8 Step 1 without purification of the product. MS (ES+) C₁₃H₉F₃N₂O₃ requires: 298. found: 299 [M+H]⁺.

### Step 2

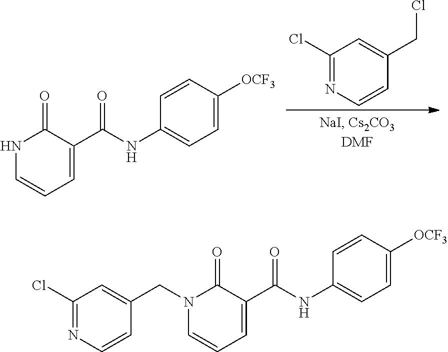 [SMILES: O=C(Nc1ccc(OC(F)(F)F)cc1)c1ccc[nH]c1=O ; ClCc1ccnc(Cl)c1 ; O=C(Nc1ccc(OC(F)(F)F)cc1)c1cccn(Cc2ccnc(Cl)c2)c1=O]

### 1-((2-Chloropyridin-4-yl)methyl)-2-oxo-N-(4-(trifluoromethoxy)phenyl)-1,2-dihydropyridine-3-carboxamide

Synthesized in an analogous method to Example 5 Step 2 (314 mg, 37%): MS (ES+) C₁₉H₁₃ClF₃N₃O₃ requires: 423. found: 424 [M+H]⁺.

### Step 3

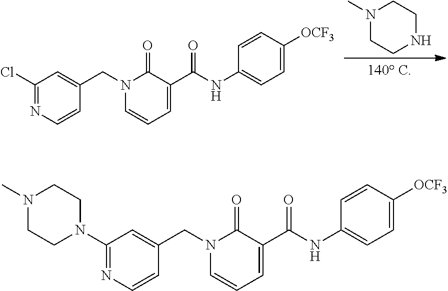

### 1-methyl-4-(4-((2-oxo-3-((4-(trifluoromethoxy)phenyl)carbamoyl)pyridin-1(2H)-yl)methyl)pyridin-1-ium-2-yl)piperazin-1-ium bis-trifluoroacetate

Synthesized in an analogous method to Example 5 Step 3 (5.7 mg, 10%). MS (ES+) C₂₄H₂₄F₃N₅O₃ requires: 487. found: 488 [M+H]⁺; ¹H-NMR (600 MHz, DMSO-d₆) δ ppm 12.1 (s, 1H), 10.1 (s, 1H), 8.52 (dd, J=7.2, 1.8 Hz, 1H), 8.29 (dd, J=7.2, 1.8 Hz, 1H), 8.11 (d, J=5.4 Hz, 1H), 7.79 (d, J=9.0 Hz, 2H), 7.35 (d, J=9.0 Hz, 2H), 6.91 (s, 1H), 6.72 (t, J=7.2 Hz, 1H), 6.57 (d, J=5.4 Hz, 1H), 5.27 (s, 2H), 4.38 (d, J=12 Hz, 2H), 3.51 (d, J=12 Hz, 2H), 3.05-3.18 (m, 4H), 2.85 (s, 3H).

### EXAMPLE 10

### 1-Methyl-4-(3-((6-methyl-2-oxo-3-((4-(trifluoromethoxy)phenyl)carbamoyl)pyridin-1(2H)-yl)methyl)phenyl)piperazin-1-ium trifluoroacetate

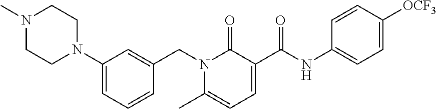 [SMILES: Cc1ccc(C(=O)Nc2ccc(OC(F)(F)F)cc2)c(=O)n1Cc1cccc(N2CCN(C)CC2)c1]

### Step 1

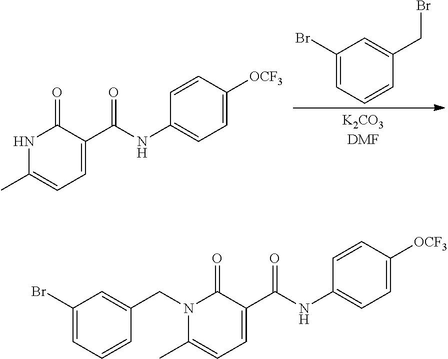 [SMILES: Cc1ccc(C(=O)Nc2ccc(OC(F)(F)F)cc2)c(=O)[nH]1 ; BrCc1cccc(Br)c1 ; Cc1ccc(C(=O)Nc2ccc(OC(F)(F)F)cc2)c(=O)n1Cc1cccc(Br)c1]

### 1-(3-Bromobenzyl)-6-methyl-2-oxo-N-(4-(trifluoromethoxy)phenyl)-1,2-dihydropyridine-3-carboxamide

Synthesized in an analogous method to Example 6 Step 1 (186 mg, 39%). MS (ES+) C₂₁H₁₆BrF₃N₂O₃ requires: 480. found: 481 [M+H]⁺.

### Step 2

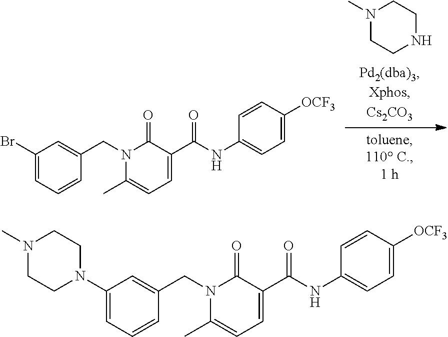

1-Methyl-4-(3-((6-methyl-2-oxo-3-((4-(trifluoromethoxy)phenyl)carbamoyl)pyridin-1(2H)-yl)methyl)phenyl)piperazin-1-ium trifluoroacetate

Synthesized in an analogous method to Example 6 Step 2 (9.7 mg, 26%); MS (ES+) C₂₆H₂₇F₃N₄O₃ requires: 500. found: 501 [M+H]⁺; ¹H-NMR (600 MHz, DMSO-d₆) δ ppm 12.99 (s, 1H), 12.04 (s, 1H), 8.56 (d, J=7.2 Hz, 1H), 7.75 (d, J=9.0 Hz, 2H), 7.28 (t, J=7.8 Hz, 1H), 7.19 (d, J=9.0 Hz, 2H), 6.84 (dd, J=1.8, 7.8 Hz, 1H), 6.69 (s, 1H), 6.67 (t, J=7.8 Hz, 1H), 6.44 (d, J=7.8 Hz, 1H), 5.42 (s, 2H), 3.68 (d, J=12 Hz, 2H), 3.62 (d, J=12 Hz, 2H), 3.30 (t, J=13.2 Hz, 2H), 3.00 (t, J=13.2 Hz, 2H), 2.88 (s, 3H), 2.43 (s, 3H).

### EXAMPLE 11

### 1-Methyl-4-(3-((2-oxo-3-((4-(trifluoromethoxy)phenyl)carbamoyl)pyridin-1(2H)-yl)methyl)phenyl)piperazin-1-ium trifluoroacetate

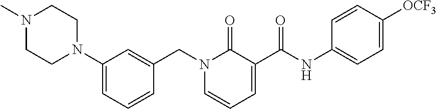 [SMILES: CN1CCN(c2cccc(Cn3cccc(C(=O)Nc4ccc(OC(F)(F)F)cc4)c3=O)c2)CC1]

### Step 1

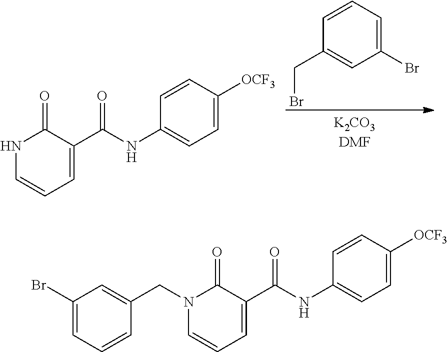 [SMILES: O=C(Nc1ccc(OC(F)(F)F)cc1)c1ccc[nH]c1=O ; BrCc1cccc(Br)c1 ; O=C(Nc1ccc(OC(F)(F)F)cc1)c1cccn(Cc2cccc(Br)c2)c1=O]

### 1-(3-Bromobenzyl)-2-oxo-N-(4-(trifluoromethoxy)phenyl)-1,2-dihydropyridine-3-carboxamide

synthesized in an analogous method to Example 6 Step 1 (157 mg, 17%). MS (ES+)C₂₀H₁₄BrF₃N₂O₃ requires: 466. found: 467 [M+H]⁺.

### Step 2

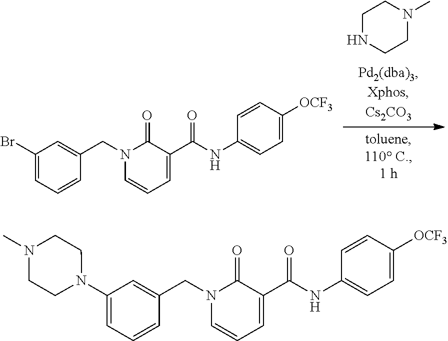

### 1-Methyl-4-(3-((2-oxo-3-((4-(trifluoromethoxy)phenyl)carbamoyl)pyridin-1(2H)-yl)methyl)phenyl)piperazin-1-ium trifluoroacetate

Synthesized in an analogous method to Example 6 Step 2 (3.7 mg, 7%). MS (ES+) C₂₅H₂₅F₃N₄O₃ requires: 486. found: 487 [M+H]⁺; ¹H-NMR (600 MHz, CD₃OD) δ ppm 8.53 (d, J=7.2 Hz, 1H), 8.05 (d, J=7.2 Hz, 1H), 7.77 (d, J=7.8 Hz, 2H), 7.29 (t, J=7.8 Hz, 1H), 7.29 (d, J=8.4 Hz, 2H), 7.05 (s, 1H), 6.98 (d, J=7.8 Hz, 1H), 6.92 (d, J=7.8 Hz, 1H), 6.60 (t, J=7.2 Hz, 1H), 5.29 (s, 2H), 3.82 (d, J=12.6 Hz, 2H), 3.58 (d, J=12.6 Hz, 2H), 3.24 (t, J=12.0 Hz, 2H), 3.04 (t, J=12.0 Hz, 2H), 2.95 (s, 3H).

### EXAMPLE 12

### 2-((2-Morpholinopyridin-4-yl)methyl)-6-(3-(4-(trifluoromethoxy)phenyl)-1,2,4-oxadiazol-5-yl)pyridazin-3(2H)-one

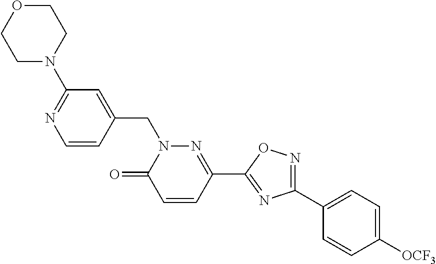 [SMILES: O=c1ccc(-c2nc(-c3ccc(OC(F)(F)F)cc3)no2)nn1Cc1ccnc(N2CCOCC2)c1]

### Step 1

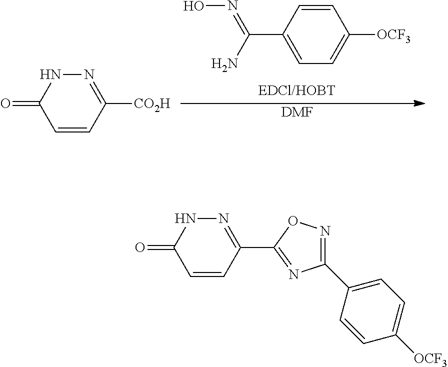

### 6-(3-(4-(Trifluoromethoxy)phenyl)1,2,4-oxadiazol-5-yl)-2H-pyridazin-3-one

1,6-Dihydro-6-oxo-3-pyridazinecarboxylic acid mono-hydrate (244 mg, 1.5 mmol) was mixed with N-hydroxy-4-(trifluoromethoxy)benzenecarboximidamide (396 mg, 1.8 mmol) in DMF (10 mL), followed by EDC.HCl (350 mg, 1.8 mmol) and HOBT (313 mg, 1.8 mmol). The mixture was stirred at RT for 30 min, then heated to 140° C. for 2 h. After cooled to RT, the reaction mixture was diluted with H₂O (40 mL), extracted with EtOAc (4×50 mL), washed with H₂O (50 mL) and brine (50 mL), dried over Na₂SO₄, filtered, and concentrated. The residue was purified by a silica gel column with (EtOAc/Hexane 20% to 100% EtOAc) to give 6-(3-(4-(trifluoromethoxy)phenyl)1,2,4-oxadiazol-5-yl)-2H-pyridazin-3-one as a yellow solid (358 mg, 74%). MS (ES+) C₁₃H₇F₃N₄O₃ requires: 324. found: 325[M+H]⁺.

### Step 2

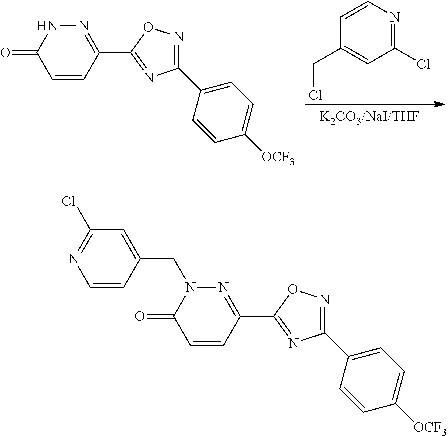 [SMILES: O=c1ccc(-c2nc(-c3ccc(OC(F)(F)F)cc3)no2)n[nH]1 ; ClCc1ccnc(Cl)c1 ; O=c1ccc(-c2nc(-c3ccc(OC(F)(F)F)cc3)no2)nn1Cc1ccnc(Cl)c1]

### 2-((2-Chloropyridin-4-yl)methyl)-6-(3-(4-(trifluoromethoxy)phenyl)-1,2,4-oxadiazol-5-yl)pyridazin-3(2H)-one

To a suspension of 6-(3-(4-(trifluoromethoxy)phenyl)1,2,4-oxadiazol-5-yl)-2H-pyridazin-3-one (142 mg, 0.44 mmol) in THF (5 mL), K₂CO₃ (180 mg, 1.3 mmol), NaI (97 mg, 0.65 mmol), and 4-(chloromethyl)-2-chloropyridine hydrochloride (130 mg, 0.65 mmol) were added at RT. The mixture was stirred at RT for 4 days. The resulting mixture was diluted with H₂O (20 mL), extracted with EtOAc (3×40 mL), washed with brine (2×50 mL) dried over Na₂SO₄, filtered, and concentrated. The residue was purified by a silica gel column (EtOAc/Hexane 16% to 80% EtOAc) to give 2-((2-chloropyridin-4-yl)methyl)-6-(3-(4-(trifluoromethoxy)phenyl)-1,2,4-oxadiazol-5-yl)pyridazin-3(2H)-one as a white solid (130 mg, 66%). MS (ES+) C₁₉H₁₁F₃N₅ClO₃ requires: 449. found: 450[M+H]⁺.

### Step 3

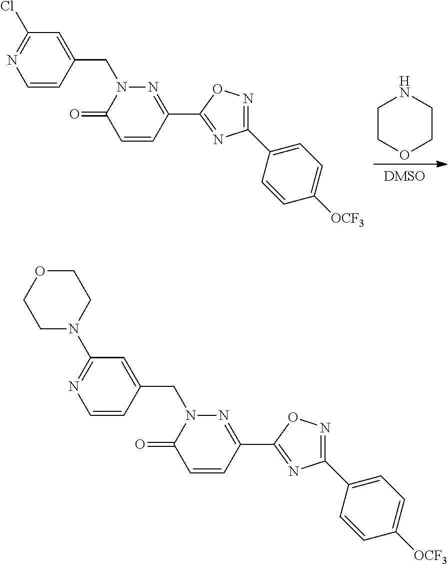 [SMILES: O=c1ccc(-c2nc(-c3ccc(OC(F)(F)F)cc3)no2)nn1Cc1ccnc(Cl)c1 ; C1COCCN1 ; O=c1ccc(-c2nc(-c3ccc(OC(F)(F)F)cc3)no2)nn1Cc1ccnc(N2CCOCC2)c1]

### 2-((2-Morpholinopyridin-4-yl)methyl)-6-(3-(4-(trifluoromethoxy)phenyl)-1,2,4-oxadiazol-5-yl)pyridazin-3(2H)-one

2-((2-Chloropyridin-4-yl)methyl)-6-(3-(4-(trifluoromethoxy)phenyl)-1,2,4-oxadiazol-5-yl)pyridazin-3(2H)-one (45 mg, 0.1 mmol) was mixed with morpholine (87 mg, 1.0 mmol) in DMSO (0.5 mL). The mixture was heated to 140° C. overnight. The resulting crude product was purified by prep-HPLC (Mobile phase: A=0.01% TFA/H₂O, B=0.01% TFA/MeCN; Gradient: B=30%-70% in 12 min; Column: C18) and then a silica gel column with (EtOAc/Hexane 16% to 100% EtOAc) to give 24(2-morpholinopyridin-4-yl)methyl)-6-(3-(4-(trifluoromethoxy)phenyl)-1,2,4-oxadiazol-5-yl)pyridazin-3(2H)-one as a white solid (3.5 mg, 7%). MS (ES+) C₂₃H₁₉F₃N₆O₄ requires: 500. found: 501[M+H]⁺; ¹H NMR (600 MHz, DMSO-d₆) δ 8.22 (d, J=8.7 Hz, 2H), 8.17 (d, J=9.7 Hz, 1H), 8.0 (d, J=5.2 Hz, 1H), 7.62 (d, J=8.5 Hz, 2H), 7.26 (d, J=9.7 Hz, 1H), 6.76 (s, 1H), 6.55 (d, J=5.1 Hz, 1H), 5.36 (s, 2H), 3.68 (t, J=4.8 Hz, 4H), 3.42 (t, J=4.8 Hz, 4H).

### EXAMPLE 13

### 2-(3-(4-Methylpiperazin-1-yl)benzyl)-6-(3-(4-(trifluoromethoxy)phenyl)-1,2,4-oxadiazol-5-yl)pyridazin-3(2H)-one

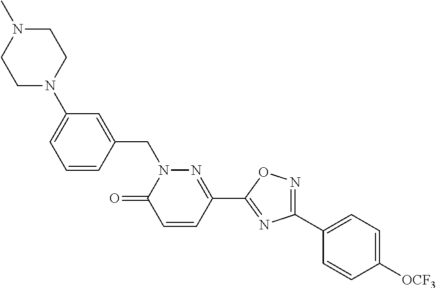 [SMILES: CN1CCN(c2cccc(Cn3nc(-c4nc(-c5ccc(OC(F)(F)F)cc5)no4)ccc3=O)c2)CC1]

### Step 1

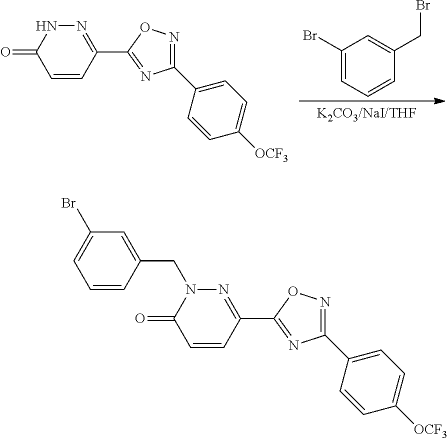 [SMILES: O=c1ccc(-c2nc(-c3ccc(OC(F)(F)F)cc3)no2)n[nH]1 ; BrCc1cccc(Br)c1 ; O=c1ccc(-c2nc(-c3ccc(OC(F)(F)F)cc3)no2)nn1Cc1cccc(Br)c1]

### 2-(3-Bromobenzyl)-6-(3-(4-(trifluoromethoxy)phenyl)-1,2,4-oxadiazol-5-yl)pyridazin-3(2H)-one

To a suspension of 6-(3-(4-(trifluoromethoxy)phenyl)1,2,4-oxadiazol-5-yl)-2H-pyridazin-3-one (324 mg, 1.0 mmol) in THF (10 mL), K₂CO₃ (276 mg, 2.0 mmol), NaI (223 mg, 1.5 mmol), and 4-(bromomethyl)benzene bromide (375 mg, 1.5 mmol) were added at RT. The mixture was stirred at 50° C. for 4 days. The resulting mixture was diluted with H₂O (40 mL), extracted with EtOAc (3×50 mL), washed with brine (2×50 mL) dried over Na₂SO₄, filtered and concentrated. The residue was purified by a silica gel column (EtOAc/Hexane 8% to 66% EtOAc) to give 2-(3-bromobenzyl)-6-(3-(4-(trifluoromethoxy)phenyl)-1,2,4-oxadiazol-5-yl)pyridazin-3(2H)-one as a white solid (330 mg, 67%). MS (ES+) C₂₀H₁₂F₃N₄BrO₃ requires: 492, 494 found: 493, 495[M+H]⁺(1:1).

### Step 2

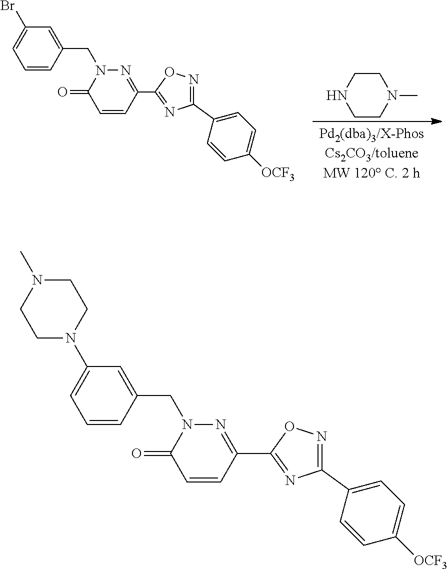

### 2-(3-(4-Methylpiperazin-1-yl)benzyl)-6-(3-(4-(trifluoromethoxy)phenyl)-1,2,4-oxadiazol-5-yl)pyridazin-3(2H)-one

2-(3-Bromobenzyl)-6-(3-(4-(trifluoromethoxy)phenyl)-1,2,4-oxadiazol-5-yl)pyridazin-3(2H)-one (50 mg, 0.1 mmol) was mixed with 1-methylpiperazine (30 mg, 0.3 mmol), Pd₂(dba)₃ (10 mg, 0.01 mmol), XPhos (19 mg, 0.04 mmol), and Cs₂CO₃ (98 mg, 0.25 mmol) in toluene (0.5 mL). The mixture was heated to 120° C. for 2 h under microwave irradiation. The resulting mixture was filtered, washed with DCM, and concentrated. The residue was purified by a silica gel column with (MeOH/EtOAc 0%-20% MeOH) to give 2-(3-(4-methylpiperazin-1-yl)benzyl)-6-(3-(4-(trifluoromethoxy)phenyl)-1,2,4-oxadiazol-5-yl)pyridazin-3(2H)-one as a white solid (7.5 mg, 15%). MS (ES+) C₂₅H₂₃F₃N₆O₃ requires: 512. found: 513[M+H]⁺; ¹H NMR (600 MHz, CDCl₃) δ 8.20 (d, J=8.8 Hz, 2H), 8.01 (d, J=9.7 Hz, 1H), 7.36 (d, J=8.2 Hz, 2H), 7.23 (t, J=7.9 Hz, 1H), 7.14 (s, 1H), 7.06 (d, J=8.7 Hz, 1H), 7.01 (d, J=7.7 Hz, 1H), 6.88 (d, J=8.2 Hz, 1H), 5.41 (s, 2H), 3.23 (t, J=4.9 Hz, 4H), 2.57 (t, J=4.9 Hz, 4H), 2.35 (s, 3H).

### EXAMPLE 14

### 2-(3-Morpholinobenzyl)-6-(3-(4-(trifluoromethoxy)phenyl)-1,2,4-oxadiazol-5-yl)pyridazin-3(2H)-one

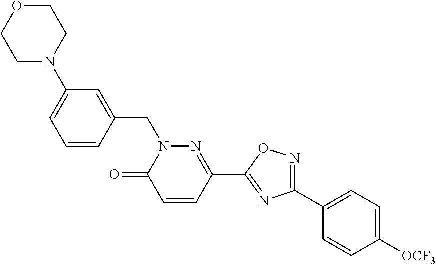 [SMILES: O=c1ccc(-c2nc(-c3ccc(OC(F)(F)F)cc3)no2)nn1Cc1cccc(N2CCOCC2)c1]

### Step 1

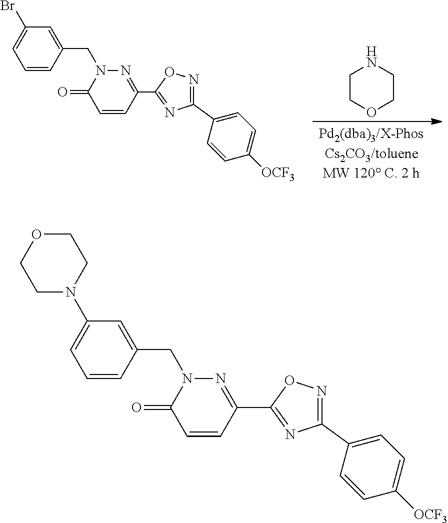 [SMILES: O=c1ccc(-c2nc(-c3ccc(OC(F)(F)F)cc3)no2)nn1Cc1cccc(Br)c1 ; C1COCCN1 ; O=c1ccc(-c2nc(-c3ccc(OC(F)(F)F)cc3)no2)nn1Cc1cccc(N2CCOCC2)c1]

### 2-(3-Morpholinobenzyl)-6-(3-(4-(trifluoromethoxy)phenyl)-1,2,4-oxadiazol-5-yl)pyridazin-3(2H)-one

Synthesized in an analogous method to Example 13, Step 2: (10 mg, 20%). MS (ES+) C₂₄H₂₀F₃N₅O₄ requires: 499. found: 500 [M+H]⁺; ¹H NMR (600 MHz, CDCl₃) δ 8.20 (d, J=8.8 Hz, 2H), 8.02 (d, J=9.7 Hz, 1H), 7.36 (d, J=8.2 Hz, 2H), 7.26 (t, J=7.9 Hz, 1H), 7.14 (s, 1H), 7.07 (d, J=9.6 Hz, 1H), 7.05 (d, J=7.6 Hz, 1H), 6.88 (d, J=8.2 Hz, 1H), 5.42 (s, 2H), 3.85 (t, J=4.9 Hz, 4H), 3.18 (t, J=4.9 Hz, 4H).

### EXAMPLE 15

### 4-((6-Methyl-2-oxo-3-(3-(4-(trifluoromethoxy)phenyl)-1,2,4-oxadiazol-5-yl)pyridin-1(2H)-yl)methyl)-2-(piperidin-1-yl)pyridin-1-ium trifluoroacetate

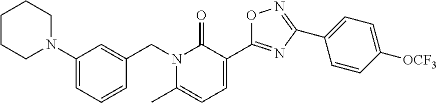 [SMILES: Cc1ccc(-c2nc(-c3ccc(OC(F)(F)F)cc3)no2)c(=O)n1Cc1cccc(N2CCCCC2)c1]

### 4-((6-Methyl-2-oxo-3-(3-(4-(trifluoromethoxy)phenyl)-1,2,4-oxadiazol-5-yl)pyridin-1(2H)-yl)methyl)-2-(piperidin-1-yl)pyridin-1-ium trifluoroacetate

Synthesized in an analogous method to Example 5 (3.0 mg, 14%): MS (ES+) C₂₆H₂₄F₃N₅O₃ requires: 511. found 512 [M+H]⁺; ¹H-NMR (600 MHz, CD₃OD) δ ppm 8.54 (d, J=8.4 Hz, 1H), 8.25 (d, J=7.8 Hz, 2H), 7.83 (d, J=6.0 Hz, 1H), 7.44 (d, J=8.4 Hz, 2H), 7.19 (s, 1H), 6.65 (d, J=6.0 Hz, 1H), 6.63 (d, J=7.8 Hz, 1H), 5.53 (s, 2H), 3.62-3.70 (m, 4H), 2.54 (s, 3H), 1.72-1.70 (m, 6H).

### EXAMPLE 16

### 4-((6-methyl-2-oxo-3-(3-(4-(trifluoromethoxy)phenyl)-1,2,4-oxadiazol-5-yl)pyridin-1(2H)-yl)methyl)-2-(pyrrolidin-1-yl)pyridin-1-ium trifluoroacetate

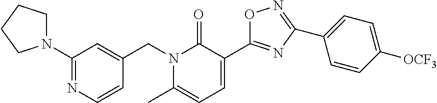 [SMILES: Cc1ccc(-c2nc(-c3ccc(OC(F)(F)F)cc3)no2)c(=O)n1Cc1ccnc(N2CCCC2)c1]

### 4-((6-methyl-2-oxo-3-(3-(4-(trifluoromethoxy)phenyl)-1,2,4-oxadiazol-5-yl)pyridin-1(2H)-yl)methyl)-2-(pyrrolidin-1-yl)pyridin-1-ium trifluoroacetate

Synthesized in an analogous method to Example 5 (2.5 mg, 11%): MS (ES+) C₂₅H₂₂F₃N₅O₃ requires: 497. found 498 [M+H]⁺; ¹H-NMR (600 MHz, CD₃OD) (δ ppm 8.55 (d, J=7.8 Hz, 1H), 8.25 (d, J=8.4 Hz, 2H), 7.82 (d, J=6.6 Hz, 1H), 7.44 (d, J=8.4 Hz, 2H), 6.83 (s, 1H), 6.71 (d, J=6.6 Hz, 1H), 6.70 (d, J=7.8 Hz, 1H), 5.54 (s, 2H), 3.52-3.58 (m, 4H), 2.54 (s, 3H), 2.10-2.17 (m, 4H).

### EXAMPLE 17

### 2-((3-Methoxypropyl)amino)-4-((6-methyl-2-oxo-3-(3-(4-(trifluoromethoxy)phenyl)-1,2,4-oxadiazol-5-yl)pyridin-1(2H)-yl)methyl)pyridin-1-ium trifluoroacetate

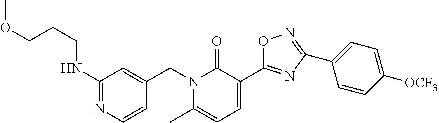 [SMILES: COCCCNc1cc(Cn2c(C)ccc(-c3nc(-c4ccc(OC(F)(F)F)cc4)no3)c2=O)ccn1]

### 2-((3-Methoxypropyl)amino)-4-((6-methyl-2-oxo-3-(3-(4-(trifluoromethoxy)phenyl)-1,2,4-oxadiazol-5-yl)pyridin-1(2H)-yl)methyl)pyridin-1-ium trifluoroacetate

Synthesized in an analogous method to Example 5 (1.4 mg, 7%): MS (ES+) C₂₅H₂₄F₃N₅O₄ requires: 515. found 516 [M+H]⁺; ¹H-NMR (600 MHz, CD₃OD) δ ppm 8.57 (d, J=7.2 Hz, 1H), 8.25 (d, J=7.2 Hz, 2H), 7.80 (d, J=6.0 Hz, 1H), 7.44 (d, J=8.4 Hz, 2H), 6.80 (d, J=6.0 Hz, 1H), 6.64 (d, J=8.4 Hz, 2H), 5.50 (s, 2H), 3.46 (t, J=6.0 Hz, 2H), 3.39 (t, J=6.0 Hz, 2H), 3.30 (s, 3H), 2.53 (s, 3H), 1.89 (t, J=6.0 Hz, 2H).

### EXAMPLE 18

### 4-((6-Methyl-2-oxo-3-(3-(4-(trifluoromethoxy)phenyl)-1,2,4-oxadiazol-5-yl)pyridin-1(2H)-yl)methyl)-2-morpholinopyridin-1-ium trifluoroacetate

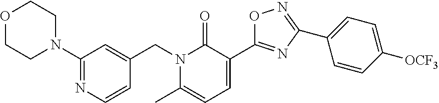 [SMILES: Cc1ccc(-c2nc(-c3ccc(OC(F)(F)F)cc3)no2)c(=O)n1Cc1ccnc(N2CCOCC2)c1]

### 4-((6-Methyl-2-oxo-3-(3-(4-(trifluoromethoxy)phenyl)-1,2,4-oxadiazol-5-yl)pyridin-1(2H)-yl)methyl)-2-morpholinopyridin-1-ium trifluoroacetate

Synthesized in an analogous method to Example 5 (1.1 mg, 6%): MS (ES+) C₂₅H₂₂F₃N₅O₄ requires: 513. found 514 [M+H]⁺; ¹H-NMR (600 MHz, CD₃OD) (δ ppm 8.54 (d, J=8.4 Hz, 1H), 8.25 (d, J=7.8 Hz, 2H), 7.95 (d, J=6.0 Hz, 1H), 7.44 (d, J=8.4 Hz, 2H), 7.08 (s, 1H), 6.72 (d, J=6.0 Hz, 1H), 6.62 (d, J=7.8 Hz, 1H), 5.54 (s, 2H), 3.82 (t, J=6.0 Hz, 4H), 3.59 (t, J=6.0 Hz, 4H), 2.53 (s, 3H).

### EXAMPLE 19

### 5-(5-methyl-4-(3-(4-methylpiperazin-1-yl)benzyl)pyrimidin-2-yl)-3-(4-(trifluoromethoxy)phenyl)-1,2,4-oxadiazole

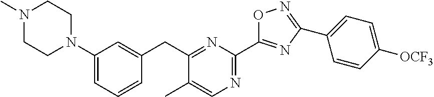 [SMILES: Cc1cnc(-c2nc(-c3ccc(OC(F)(F)F)cc3)no2)nc1Cc1cccc(N2CCN(C)CC2)c1]

### Step 1

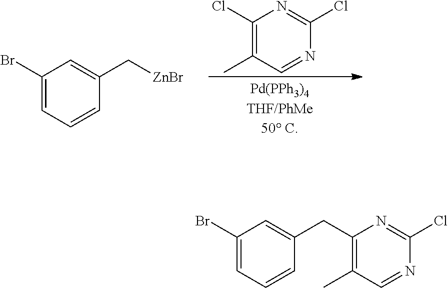

### 4-(3-bromobenzyl)-2-chloro-5-methylpyrimidine

A mixture of Pd(PPh₃)₄ (0.32 g, 0.27 mmol) and 2,4-dichloro-5-methylpyrimidine (1.3 g, 8.0 mmol) in toluene was degassed. (3-bromobenzyl)zinc(II) bromide (11 ml, 5.5 mmol) solution was then added. The reaction mixture was stirred at 50° C. for 18 hours. The reaction was diluted with EtOAc and Water. NH₄OH was then added. The reaction was stirred for 30 mins. The organic layer was separated, concentrated, and loaded to Biotage for purification (0-50% EtOAc in Hexanes) to deliver the title compound (880 mg, 54%) as a colorless liquid. MS(ES⁺) C₁₂H₁₀BrClN₂ requires: 296, 298 found: 297, 299 [M+2+H]⁺(1:1).

### Step 2

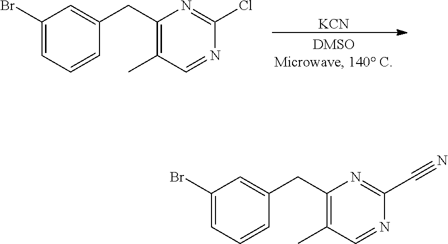 [SMILES: Cc1cnc(Cl)nc1Cc1cccc(Br)c1 ; "KCN" ; Cc1cnc(C#N)nc1Cc1cccc(Br)c1]

### 4-(3-bromobenzyl)-5-methylpyrimidine-2-carbonitrile

A solution of 4-(3-bromobenzyl)-2-chloro-5-methylpyrimidine (677 mg, 2.3 mmol), and cyanopotassium (178 mg, 2.7 mmol) in DMSO (10 ml) was heated to 140° C. under microwave for 1 hour. The reaction mixture was diluted with hexanes and EtOAc, and washed with brine. The organic layer was concentrated and purified by Biotage (10-50% EtOAc in Hexanes) to give the title compound (350 mg, 53%) as a white solid. MS(ES⁺) C₁₃H₁₀BrN₃ requires: 287, 289 found: 288, 290 [M+2+H]⁺(1:1).

### Step 3

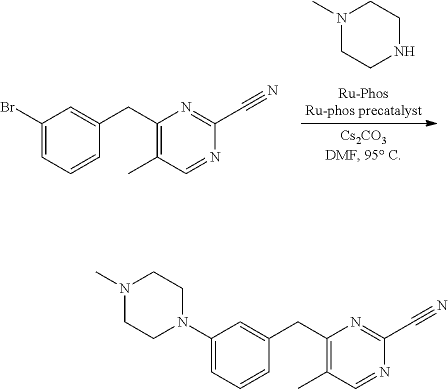

### 5-methyl-4-(3-(4-methylpiperazin-1-yl)benzyl)pyrimidine-2-carbonitrile

A mixture of Ru-phos (37.2 mg, 0.08 mmol), Ru-phos precatalyst (42.5 mg, 0.06 mmol), cesium carbonate (249 mg, 0.764 mmol) was placed in a vial charged with a stir bar. The vial was degassed and filled with nitrogen (three cycles). DMF (2.5 mL) was added and the vial was degassed and filled with nitrogen. The reaction was stirred at 95° C. for 10 mins and then cooled down to room temperature. Another vial containing 4-(3-bromobenzyl)-5-methylpyrimidine-2-carbonitrile (100 mg, 0.35 mmol) was degassed and filled with nitrogen. DMF (2.5 mL) was added, followed by 1-methylpiperazine (0.104 ml, 0.94 mmol). The vial was degassed again and filled with nitrogen. The solution was subsequently transferred to the first reaction vial. The reaction mixture was then heated to 95° C. for 75 mins. The reaction mixture was diluted with dichloromethane and washed with brine. The organic layer was concentrated and purified by Biotage (0-15% methanol in dichloromethane) to give the title compound (98 mg, 60%) as a brownish oil. MS (ES+) C₁₈H₂₁N₅ requires: 307 found: 308 [M+H]⁺.

### Step 4

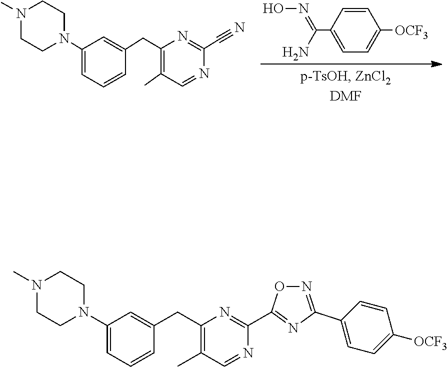 [SMILES: Cc1cnc(C#N)nc1Cc1cccc(N2CCN(C)CC2)c1 ; N/C(=N\O)c1ccc(OC(F)(F)F)cc1 ; Cc1cnc(-c2nc(-c3ccc(OC(F)(F)F)cc3)no2)nc1Cc1cccc(N2CCN(C)CC2)c1]

### 5-(5-methyl-4-(3-(4-methylpiperazin-1-yl)benzyl)pyrimidin-2-yl)-3-(4-(trifluoromethoxy)phenyl)-1,2,4-oxadiazole

A mixture of pTSA.H₂O (23.5 mg, 0.124 mmol), ZnCl₂ (124 μl, 1.0 M solution in DMF, 0.124 mmol), and (Z)—N′-hydroxy-4-(trifluoromethoxy)benzimidamide (27.2 mg, 0.124 mmol) was heated to 100° C. for 40 minutes. The reaction mixture was then diluted with EtOAc and washed with NH₄OH solution. The organic layer was concentrated and purified by preparative HPLC (20-60% acetonitrile in H₂O) to give the title compound (3.1 mg, 8.4%). MS(ES⁺) C₂₆H₂₅F₃N₆O₂ requires: 510. found: 511 [M+H]⁺; ¹H-NMR (600 MHz, CD₃OD) δ ppm 8.76 (s, 1H), 8.31 (d, J=7.2 Hz, 2H), 7.49 (d, J=7.2 Hz, 2H), 7.23 (t, J=6.0 Hz, 1H), 7.03 (s, 1H), 6.90 (dd, J=7.8, 2.4 Hz, 1H), 6.86 (d, J=7.8 Hz, 1H), 4.31 (s, 2H), 3.81 (d, J=13.2 Hz, 2H), 3.56 (d, J=13.2 Hz, 2H), 3.23 (t, J=12.0 Hz, 2H), 3.01 (t, J=12.0 Hz, 2H), 2.93 (s, 3H), 2.39 (s, 3H).

### EXAMPLE 20

### 1-((2-(4-methylpiperazin-1-yl)pyridin-4-yl)methyl)-3-(3-(4-(trifluoromethoxy)phenyl)-1,2,4-oxadiazol-5-yl)pyridin-2(1H)-one

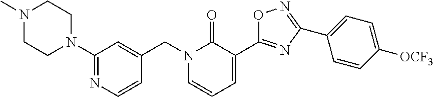 [SMILES: CN1CCN(c2cc(Cn3cccc(-c4nc(-c5ccc(OC(F)(F)F)cc5)no4)c3=O)ccn2)CC1]

### Step 1

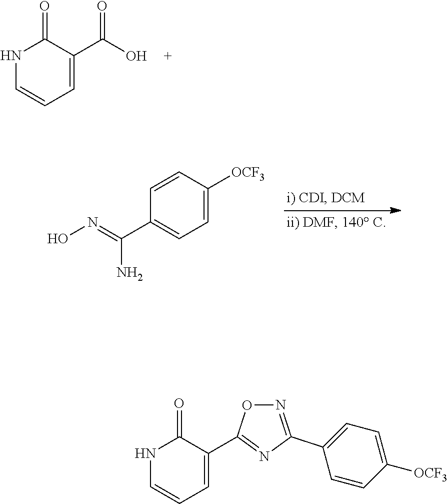 [SMILES: O=C(O)c1ccc[nH]c1=O ; N/C(=N\O)c1ccc(OC(F)(F)F)cc1 ; O=c1[nH]cccc1-c1nc(-c2ccc(OC(F)(F)F)cc2)no1]

### 3-(3-(4-(Trifluoromethoxy)phenyl)-1,2,4-oxadiazol-5-yl)pyridin-2(1H)-one

2-Oxo-1,2-dihydropyridine-3-carboxylic acid (501 mg, 3.6 mmol) and CDI (584 mg, 3.6 mmol) in DCM (10 ml) was stirred at RT for 4 hours. (Z)—N′-Hydroxy-4-(trifluoromethoxy)benzimidamide (660 mg, 3 mmol) was added. The reaction mixture was stirred at 45° C. for 12 hours, then DMF (5 mL) was added. The mixture was concentrated to remove DCM. Another portion of DMF (15 mL) was added. The reaction mixture was then heated to 140° C. for 60 minutes. DCM was added to precipitate a white solid. The mixture was filtered to give the title compound (530 mg, 55%) as a solid, which was used for the next step without further purification. MS(ES⁺) C₁₄H₈F₃N₃O₃ requires: 323. found: 324 [M+H]⁺.

### Step 2

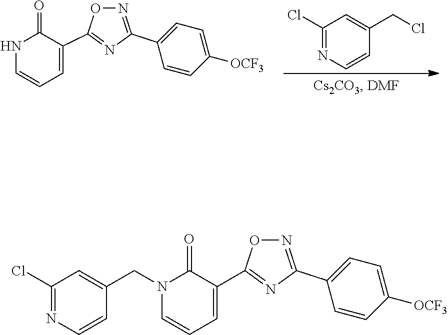 [SMILES: O=c1[nH]cccc1-c1nc(-c2ccc(OC(F)(F)F)cc2)no1 ; ClCc1ccnc(Cl)c1 ; O=c1c(-c2nc(-c3ccc(OC(F)(F)F)cc3)no2)cccn1Cc1ccnc(Cl)c1]

### 1-((2-Chloropyridin-4-yl)methyl)-3-(3-(4-(trifluoromethoxy)phenyl)-1,2,4-oxadiazol-5-yl)pyridin-2(1H)-one

Synthesized in an analogous method to Example 5 (223 mg, 40%): MS(ES⁺) C₂₀H₁₂ClF₃N₄O₃ requires: 448/450. found: 449/451 [M+H]⁺.

### Step 3

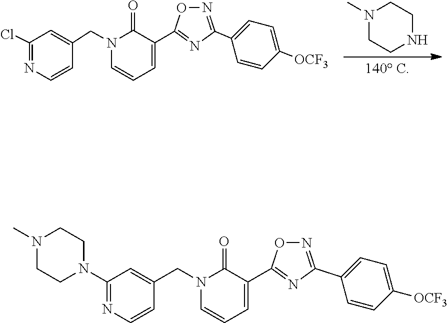

### 1-((2-(4-Methylpiperazin-1-yl)pyridin-4-yl)methyl)-3-(3-(4-(trifluoromethoxy)phenyl)-1,2,4-oxadiazol-5-yl)pyridin-2(1H)-one

Synthesized in an analogous method to Example 5 (7.8 mg, 76%). MS(ES⁺) C₂₅H₂₃F₃N₆O₃ requires: 512. found: 513 [M+H]⁺.

### EXAMPLE 21

### 6-methyl-1-((2-(4-methylpiperazin-1-yl)pyridin-4-yl)methyl)-3-(5-(4-(trifluoromethoxy)phenyl)-1,3,4-oxadiazol-2-yl)pyridin-2(1H)-one

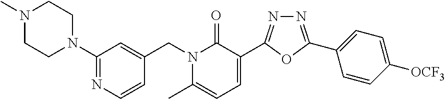 [SMILES: Cc1ccc(-c2nnc(-c3ccc(OC(F)(F)F)cc3)o2)c(=O)n1Cc1ccnc(N2CCN(C)CC2)c1]

### Step 1

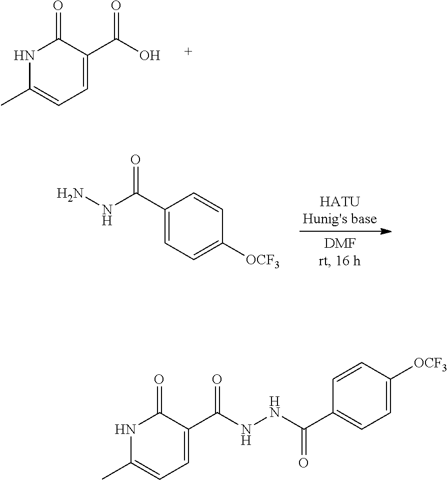 [SMILES: Cc1ccc(C(=O)O)c(=O)[nH]1 ; NNC(=O)c1ccc(OC(F)(F)F)cc1 ; Cc1ccc(C(=O)NNC(=O)c2ccc(OC(F)(F)F)cc2)c(=O)[nH]1]

### 6-Methyl-2-oxo-N′-(4-(trifluoromethoxy)benzoyl)-1,2-dihydropyridine-3-carbohydrazide

A reaction vial was charged with 6-methyl-2-oxo-1,2-dihydropyridine-3-carboxylic acid (500 mg, 3.27 mmol), 4-(trifluoromethoxy)benzohydrazide (719 mg, 3.27 mmol), Hunig's Base (0.85 mL, 4.9 mmol), HATU (1.61 g, 4.24 mmol) and DMF. The reaction mixture was stirred at RT for 16 hours. The reaction mixture was filtered, and the solids were washed with DCM. The filtrate was concentrated under reduced pressure, the residue was taken up in DCM and the solids were filtered. The combined solids were washed with DCM, air-dried and the azeotroped with toluene (3×30 mL). The product was used in the next step without further purification (413 mg, 36%). MS (ES+) C₁₅H₁₂F₃N₃O₄ requires: 355. found: 356 [M+H]⁺; ¹H NMR (600 MHz, DMSO-d₆) δ12.69 (s, 1H), 11.50 (d, J=2.1 Hz, 1H), 10.96 (d, J=2.1 Hz, 1H), 8.28 (d, J=7.4 Hz, 1H), 8.02 (d, J=8.4 Hz, 2H), 7.52 (d, J=8.4 Hz, 2H), 6.38 (d, J=7.4 Hz, 1H), 2.30 (s, 3H).

### Step 2

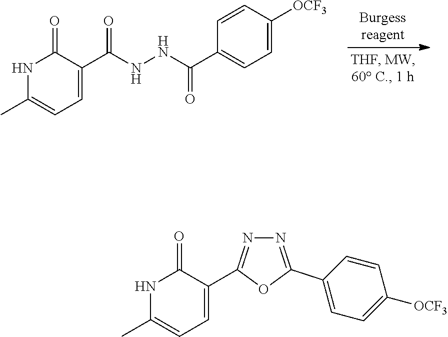 [SMILES: Cc1ccc(C(=O)NNC(=O)c2ccc(OC(F)(F)F)cc2)c(=O)[nH]1 ; Cc1ccc(-c2nnc(-c3ccc(OC(F)(F)F)cc3)o2)c(=O)[nH]1]

### 6-Methyl-3-(5-(4-(trifluoromethoxy)phenyl)-1,3,4-oxadiazol-2-yl)pyridin-2(1H)-one

To a solution of 6-methyl-2-oxo-N′-(4-(trifluoromethoxy)benzoyl)-1,2-dihydropyridine-3-carbohydrazide (200 mg, 0.56 mmol) in THF (5.6 mL) was added Burgess reagent (376 mg, 1.57 mmol) and the reaction mixture was put into the microwave and heated at 60° C. for 1 hour. The reaction mixture was allowed to cool to RT and the solvent was evaporated. The residue was purified by Biotage (25 g SNAP, 2% to 30% MeOH in DCM) to provide the title compound (177 mg, 84%). MS (ES+) C₁₅H₁₀F₃N₃O₃ requires: 337. found: 338 [M+H]⁺; ¹H NMR (600 MHz, DMSO-d₆) δ12.38 (s, 1H), 8.22 (d, J=7.2 Hz, 1H), 8.19 (d, J=8.8 Hz, 2H), 7.64 (d, J=8.8 Hz, 2H), 6.28 (d, J=7.2 Hz, 1H), 2.31 (s, 3H).

### Step 3

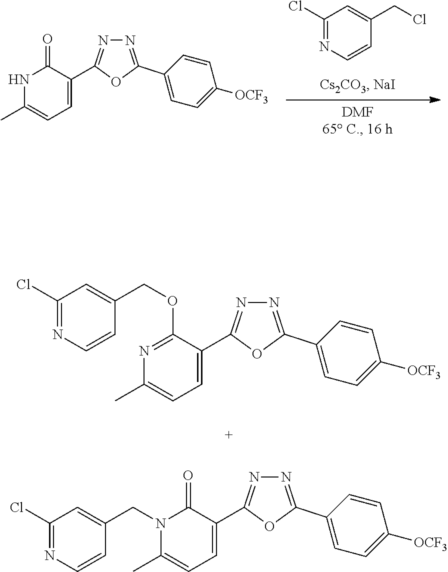 [SMILES: Cc1ccc(-c2nnc(-c3ccc(OC(F)(F)F)cc3)o2)c(=O)[nH]1 ; ClCc1ccnc(Cl)c1 ; Cc1ccc(-c2nnc(-c3ccc(OC(F)(F)F)cc3)o2)c(OCc2ccnc(Cl)c2)n1 ; Cc1ccc(-c2nnc(-c3ccc(OC(F)(F)F)cc3)o2)c(=O)n1Cc1ccnc(Cl)c1]

### 1-((2-Chloropyridin-4-yl)methyl)-6-methyl-3-(5-(4-(trifluoromethoxy)phenyl)-1,3,4-oxadiazol-2-yl)pyridin-2(1H)-one

To a solution of 6-methyl-3-(5-(4-(trifluoromethoxy)phenyl)-1,3,4-oxadiazol-2-yl)pyridin-2(1H)-one (177 mg, 0.52 mmol) in DMF (10 mL) was added Cs₂CO₃ (652 mg, 2.0 mmol), NaI (15 mg, 0.10 mmol) and 2-chloro-4-(chloromethyl)pyridine (172 μl, 1.40 mmol). The reaction mixture was heated at 65° C. for 16 hours. The reaction mixture was allowed to cool to RT, brine was added, and the aqueous phase was extracted with EtOAc (3×10 mL). The combined organic phases were dried over Na₂SO₄, filtered, and the solvent was evaporated. The residue was purified by Biotage (SNAP 25, 20% to 100% EtOAc in hexanes, followed by 2% to 30% MeOH in DCM) to provide 2-(2-((2-chloropyridin-4-yl)methoxy)-6-methylpyridin-3-yl)-5-(4-(trifluoromethoxy)phenyl)-1,3,4-oxadiazole (132 mg, 28%), and 1-((2-chloropyridin-4-yl)methyl)-6-methyl-3-(5-(4-(trifluoromethoxy)phenyl)-1,3,4-oxadiazol-2-yl)pyridin-2(1H)-one (183 mg, 39%). MS (ES+)₂-(2-((2-chloropyridin-4-yl)methoxy)-6-methylpyridin-3-yl)-5-(4-(trifluoromethoxy)phenyl)-1,3,4-oxadiazole C₂₁H₁₄ClF₃N₄O₃ requires: 462/464. found: 463/465 [M+H]⁺; 1-((2-chloropyridin-4-yl)methyl)-6-methyl-3-(5-(4-(trifluoromethoxy)phenyl)-1,3,4-oxadiazol-2-yl)pyridin-2(1H)-one MS (ES+) C₂₁H₁₄ClF₃N₄O₃ requires: 462. found: 463 [M+H]⁺; ¹H NMR (600 MHz, MeOH-d₄) δ8.36 (d, J=5.3 Hz, 1H), 8.34 (d, J=7.4 Hz, 1H), 8.19 (d, J=7.7 Hz, 2H), 7.33 (d, J=7.7 Hz, 2H), 7.26 (s, 1H), 7.05 (d, J=5.3 Hz, 1H), 6.35 (d, J=7.4 Hz, 1H), 5.42 (s, 2H), 2.31 (s, 3H).

### Step 4

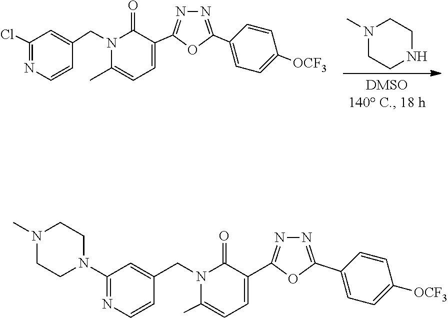

### 6-Methyl-1-((2-(4-methylpiperazin-1-yl)pyridin-4-yl)methyl)-3-(5-(4-(trifluoromethoxy)phenyl)-1,3,4-oxadiazol-2-yl)pyridin-2(1H)-one

To a solution of 1-((2-chloropyridin-4-yl)methyl)-6-methyl-3-(5-(4-(trifluoromethoxy)phenyl)-1,3,4-oxadiazol-2-yl)pyridin-2(1H)-one (40 mg, 0.086 mmol) in DMSO (500 μl) was added 1-methylpiperazine (49 μl, 0.43 mmol). The reaction mixture was heated to 140° C. for 12 hours. Additional 1-methylpiperazine (49 μl, 0.43 mmol) was added and the reaction mixture was heated to 150° C. for 6 hours. The reaction mixture was cooled to room temperature, filtered and purified by Mass-triggered RP-HPLC. The product was neutralized by washing it through a Bond Elut-SCX ion exchange column (50 g, Agilent) to provide the title compound (2.8 mg, 6%); MS (ES+) C₂₆H₂₅F₃N₆O₃ requires: 526. found: 527 [M+H]+; ¹H NMR (600 MHz, CDCl₃) δ 8.32 (d, J=7.7 Hz, 1H), 8.20 (d, J=9.05 Hz, 2H), 8.13 (d, J=5.5 Hz, 1H), 7.33 (d, J=9.05 Hz, 2H), 6.41 (s, 1H), 6.32 (d, J=7.7 Hz, 1H), 6.35 (d, J=5.5 Hz, 1H), 5.37 (s, 2H), 3.64 (m, 4H), 2.72 (m, 4H), 2.49 (s, 3H), 2.41 (s, 3H).

### EXAMPLE 22

### 3-((6-Methyl-2-oxo-3-(3-(4-(trifluoromethoxy)phenyl)-1,2,4-oxadiazol-5-yl)pyridin-1(2H)-yl)methyl)benzamide

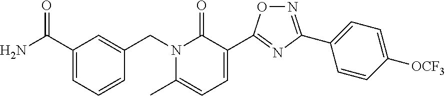 [SMILES: Cc1ccc(-c2nc(-c3ccc(OC(F)(F)F)cc3)no2)c(=O)n1Cc1cccc(C(N)=O)c1]

### Step 1

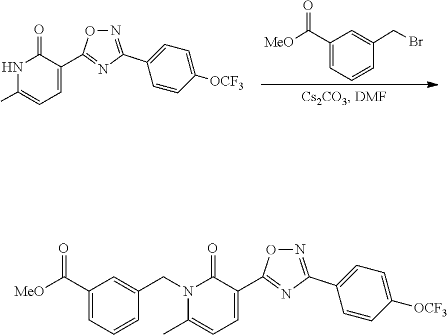 [SMILES: Cc1ccc(-c2nc(-c3ccc(OC(F)(F)F)cc3)no2)c(=O)[nH]1 ; COC(=O)c1cccc(CBr)c1 ; COC(=O)c1cccc(Cn2c(C)ccc(-c3nc(-c4ccc(OC(F)(F)F)cc4)no3)c2=O)c1]

### Methyl 3-((6-methyl-2-oxo-3-(3-(4-(trifluoromethoxy)phenyl)-1,2,4-oxadiazol-5-yl)pyridin-1(2H)-yl)methyl)benzoate

6-Methyl-3-(3-(4-(trifluoromethoxy)phenyl)-1,2,4-oxadiazol-5-yl)pyridin-2(1H)-one (200 mg, 0.593 mmol), methyl 3-(bromomethyl)benzoate (177 mg, 0.771 mmol), and Cs₂CO₃ (251 mg, 0.771 mmol) in DMF (1 ml) was stirred at RT for 2 hours. The reaction was diluted with EtOAc, washed with brine, and concentrated to give a crude product, which was purified by Biotage (10-70% ethyl acetate in hexanes) to give the title compound (196 mg, 68%) as a solid. MS(ES⁺) C₂₄H₁₈F₃N₃O₅ requires: 485. found 486 [M+H]⁺.

### Step 2

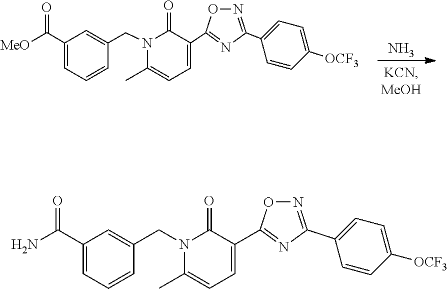

### 3-((6-Methyl-2-oxo-3-(3-(4-(trifluoromethoxy)phenyl)-1,2,4-oxadiazol-5-yl)pyridin-1(2H)-yl)methyl)benzamide

A mixture of KCN (2.01 mg, 0.03 mmol), ammonia (0.44 ml, 3.09 mmol), and methyl 3-((6-methyl-2-oxo-3-(3-(4-(trifluoromethoxy)phenyl)-1,2,4-oxadiazol-5-yl)pyridin-1(2H)-yl)methyl)benzoate (15.00 mg, 0.031 mmol) in THF (0.4 ml) was heated to 100° C. under microwave for 10 hours. The reaction was diluted with EtOAc, washed with brine, and concentrated to give a crude product, which was purified by prep HPLC (30-70% MeCN in H₂O) to give the title compound (1.9 mg 13%) as a solid. MS(ES⁺) C₂₃H₁₇F₃N₄O₄ requires: 470. found 471 [M+H]⁺; ¹H-NMR (600 MHz, CD₃OD) δ ppm 8.50 (d, J=7.8 Hz, 1H), 8.26 (m, 2H), 8.12 (d, J=5.4 Hz, 1H), 7.79 (m, 1H), 7.73 (s, 1H), 7.44 (m, 4H), 6.56 (d, J=7.8 Hz, 1H), 5.59 (s, 2H), 2.51 (s, 3H).

### EXAMPLE 23

### N-Methyl-3-((6-methyl-2-oxo-3-(3-(4-(trifluoromethoxy)phenyl)-1,2,4-oxadiazol-5-yl)pyridin-1(2H)-yl)methyl)benzamide

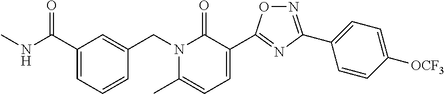 [SMILES: CNC(=O)c1cccc(Cn2c(C)ccc(-c3nc(-c4ccc(OC(F)(F)F)cc4)no3)c2=O)c1]

### Step 1

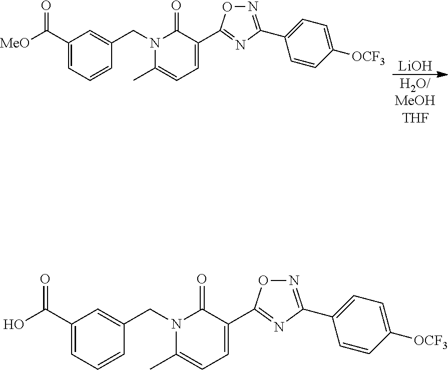 [SMILES: COC(=O)c1cccc(Cn2c(C)ccc(-c3nc(-c4ccc(OC(F)(F)F)cc4)no3)c2=O)c1 ; "LiOH" ; "H2O/MeOHTHF" ; Cc1ccc(-c2nc(-c3ccc(OC(F)(F)F)cc3)no2)c(=O)n1Cc1cccc(C(=O)O)c1]

### 3-((6-Methyl-2-oxo-3-(3-(4-(trifluoromethoxy)phenyl)-1,2,4-oxadiazol-5-yl)pyridin-1(2H)-yl)methyl)benzoic acid

LiOH (4.9 mg, 0.21 mmol) and methyl 3-((6-methyl-2-oxo-3-(3-(4-(trifluoromethoxy)phenyl)-1,2,4-oxadiazol-5-yl)pyridin-1(2H)-yl)methyl)benzoate (20 mg, 0.04 mmol) in a mixing solvent of THF (0.4 ml), MeOH (0.2 ml) and H₂O (0.1 ml) was stirred at RT for 30 minutes. The reaction was diluted with EtOAc, washed with 1N HCl, anc concentrated to give a crude product, which was used for the next step without further purification. An aliquot was purified by prep HPLC (40-60% MeCN in H₂O) to give the title compound for test. MS(ES⁺) C₂₃H₁₆F₃N₃O₅ requires: 471. found 472 [M+H]⁺.

### Step 2

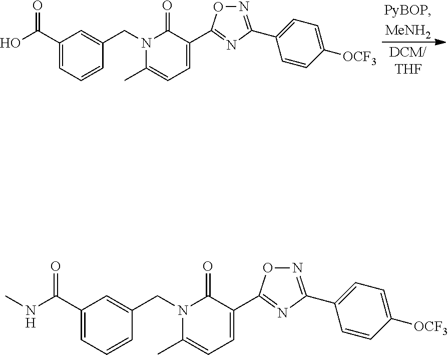 [SMILES: Cc1ccc(-c2nc(-c3ccc(OC(F)(F)F)cc3)no2)c(=O)n1Cc1cccc(C(=O)O)c1 ; "DCM/THF" ; "PyBOP,MeNH2" ; CNC(=O)c1cccc(Cn2c(C)ccc(-c3nc(-c4ccc(OC(F)(F)F)cc4)no3)c2=O)c1]

### N-Methyl-3-((6-methyl-2-oxo-3-(3-(4-(trifluoromethoxy)phenyl)-1,2,4-oxadiazol-5-yl)pyridin-1(2H)-yl)methyl)benzamide

PyBOP (31.5 mg, 0.06 mmol), N-ethyl-N-isopropylpropan-2-amine (0.036 ml, 0.20 mmol), and 3-((6-methyl-2-oxo-3-(3-(4-(trifluoromethoxy)phenyl)-1,2,4-oxadiazol-5-yl)pyridin-1(2H)-yl)methyl)benzoic acid (19 mg, 0.04 mmol) in DCM (0.2 ml) was stirred at RT for 10 mins. Then a solution dimethylamine (0.20 ml, 0.4 mmol) in THF (0.2 ml) was added. The mixture was stirred at RT for 30 mins. The reaction was diluted with MeOH and concentrated to give a crude product, which was purified by prep HPLC (40-80% MeCN in H₂O) to give the title compound (13.8 mg, 69%) as a solid: MS(ES⁺) C₂₄H₁₉F₃N₄O₄ requires: 484. found: 485 [M+H]⁺; ¹H-NMR (600 MHz, CD₃OD) δ ppm 8.48 (d, J=7.8 Hz, 1H), 8.24 (m, 2H), 8.72 (d, J=6.6 Hz, 1H), 7.66 (s, 1H), 7.44 (m, 4H), 6.54 (d, J=7.8 Hz, 1H), 5.57 (s, 2H), 2.87 (s, 3H), 2.49 (s, 3H).

### EXAMPLE 24

### 2-(4-Methylbenzyl)-6-(3-(4-(trifluoromethoxy)phenyl)-1,2,4-oxadiazol-5-yl)pyridazin-3(2H)-one

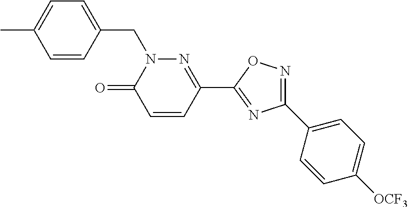 [SMILES: Cc1ccc(Cn2nc(-c3nc(-c4ccc(OC(F)(F)F)cc4)no3)ccc2=O)cc1]

### Step 1

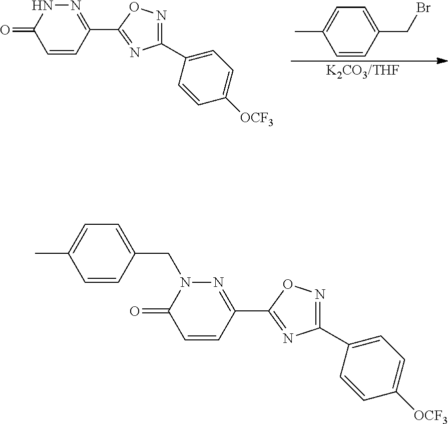

### 2-(4-Methylbenzyl)-6-(3-(4-(trifluoromethoxy)phenyl)-1,2,4-oxadiazol-5-yl)pyridazin-3(2H)-one

To a suspension of 6-(3-(4-(trifluoromethoxy)phenyl)1,2,4-oxadiazol-5-yl)-2H-pyridazin-3-one (65 mg, 0.2 mmol) in THF (1 mL), K₂CO₃ (55 mg, 0.4 mmol) and -bromo-p-xylene (56 mg, 0.3 mmol) were added at RT. The mixture was stirred at 50° C. for 24 h. The resulting mixture was diluted with H₂O (10 mL), extracted with EtOAc (3×20 mL), washed with brine (2×20 mL) dried over Na₂SO₄, filtered and concentrated. The residue was purified by a silica gel column (EtOAc/Hexane 5% to 50% EtOAc) to give 2-(4-methylbenzyl)-6-(3-(4-(trifluoromethoxy)phenyl)-1,2,4-oxadiazol-5-yl)pyridazin-3(2H)-one as a white solid (65 mg, 76%). MS (ES+) C₂₁H₁₅F₃N₄O₃ requires: 428. found: 429 [M+H]⁺. ¹H NMR (600 MHz, CDCl₃) δ 8.20 (d, J=8.8 Hz, 2H), 8.01 (d, J=9.6 Hz, 1H), 7.43 (d, J=8.0 Hz, 2H), 7.36 (d, J=8.8 Hz, 2H), 7.16 (d, J=7.9 Hz, 2H), 7.06 (d, J=9.6 Hz, 1H), 5.43 (s, 2H), 2.33 (s, 3H).

### EXAMPLE 25

### 3-((2-oxo-5-(3-(4-(trifluoromethoxy)phenyl)-1,2,4-oxadiazol-5-yl)pyridin-1(2H)-yl)methyl)benzamide

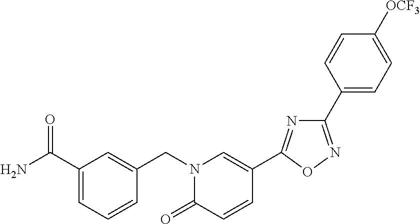 [SMILES: NC(=O)c1cccc(Cn2cc(-c3nc(-c4ccc(OC(F)(F)F)cc4)no3)ccc2=O)c1]

### Step 1

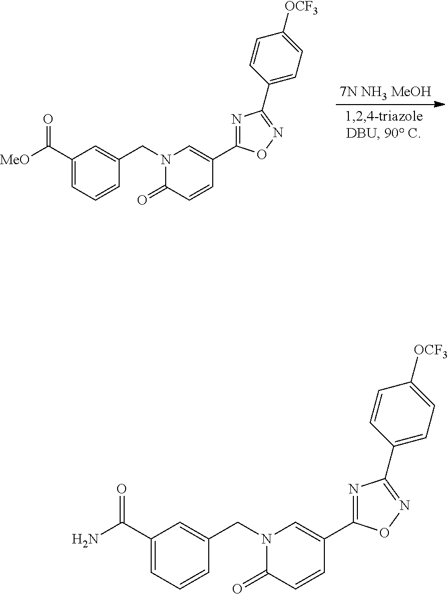 [SMILES: COC(=O)c1cccc(Cn2cc(-c3nc(-c4ccc(OC(F)(F)F)cc4)no3)ccc2=O)c1 ; NC(=O)c1cccc(Cn2cc(-c3nc(-c4ccc(OC(F)(F)F)cc4)no3)ccc2=O)c1]

### 3-((2-oxo-5-(3-(4-(trifluoromethoxy)phenyl)-1,2,4-oxadiazol-5-yl)pyridin-1(2H)-yl)methyl)benzamide

To a mixture of methyl 3-((2-oxo-5-(3-(4-(trifluoromethoxy)phenyl)-1,2,4-oxadiazol-5-yl)pyridin-1(2H)-yl)methyl)benzoate (Prepared according to example 26) (10.0 mg, 0.0212 mmol), 1,2,4-triazole (14.7 mg, 0.212 mmol), and DBU (0.032 mL, 0.21 mmol) was added 7 N NH₃ in MeOH (0.30 mL, 2.1 mmol). The mixture was heated in a sealed vial to 90° C. for 12 h, cooled to RT, and concentrated under reduced pressure. The crude oil was purified by Biotage (100% DCM to 20% MeOH/DCM) to provide a white solid (1.2 mg, 12%): ¹H NMR (600 MHz, CDCl₃) δ ppm 8.39 (d, J=2.2 Hz, 1H), 8.16 (app. dt, J=9.0 Hz, 2.0 Hz, 2H), 8.03 (dd, J=9.6 Hz, 2.5 Hz, 1H), 7.89 (app. t, J=1.5 Hz, 1H), 7.77 (dt, J=7.9 Hz, 1.4 Hz, 1H), 7.58 (d, J=7.8 Hz, 1H), 7.49 (t, J=7.8 Hz, 1H), 7.35 (d, J=8.1 Hz, 2H), 6.76 (d, J=9.3 Hz, 1H), 5.45 (s, 2H); MS(ES⁺) C₂₂H₁₅F₃N₄O₄ requires: 456. found: 457 [M+H]⁺.

### EXAMPLE 26

### 1-(4-Methylbenzyl)-5-(3-(4-(trifluoromethoxy)phenyl)-1,2,4-oxadiazol-5-yl)pyridin-2(1H)-one

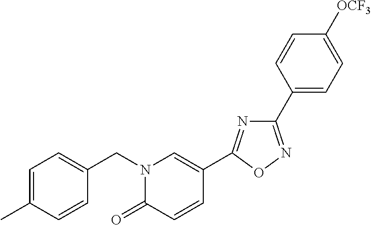 [SMILES: Cc1ccc(Cn2cc(-c3nc(-c4ccc(OC(F)(F)F)cc4)no3)ccc2=O)cc1]

### Step 1

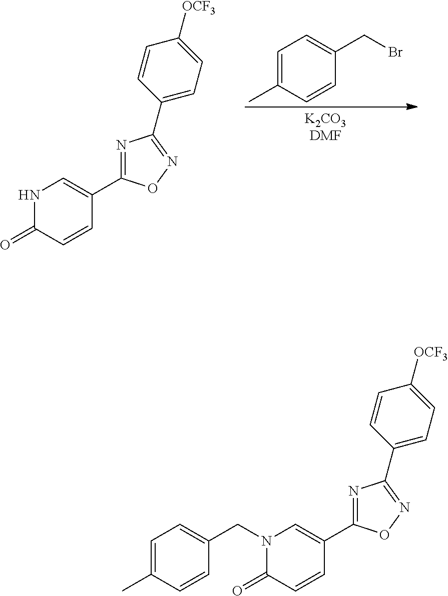

### 1-(4-Methylbenzyl)-5-(3-(4-(trifluoromethoxy)phenyl)-1,2,4-oxadiazol-5-yl)pyridin-2(1H)-one

To a suspension of K₂CO₃ (19.24 mg, 0.139 mmol) and 5-(3-(4-(trifluoromethoxy)phenyl)-1,2,4-oxadiazol-5-yl)pyridin-2(1H)-one (Example 7 Step 1) (30 mg, 0.093 mmol) in DMF (1.5 mL) was added 1-(bromomethyl)-4-methylbenzene (20.6 mg, 0.11 mmol) and the mixture was stirred at RT 2 h. The mixture was partitioned between EtOAC (2 mL) and H₂O (2 mL). The aqueous layer was extracted with EtOAc (3×2 mL) and the combined organic layers were washed with H₂O (3×2 mL) and brine (2 mL), dried (Na₂SO₄), filtered, and concentrated under reduced pressure. The crude oil was purified by Biotage (100% Hex to 30% EtOAc/Hex) to provide a white solid (25 mg, 63% 2-steps): ¹H NMR (600 MHz, CDCl₃) δ ppm 8.33 (d, J=2.3 Hz, 1H), 8.16-8.12 (m, 2H), 8.0 (dd, J=12.2 Hz, 9.5 Hz, 1H), 7.34 (d, J=8.1 Hz, 2H), 7.28 (d, J=8.1 Hz, 2H), 7.20 (d, J=7.9 Hz, 2H), 6.73 (d, J=9.5 Hz, 1H), 5.20 (s, 2H), 2.35 (s, 3H); MS(ES⁺) C₂₂H₁₆F₃N₃O₃ requires: 427. found: 428 [M+H]⁺.

### EXAMPLE 27

### 6-Methyl-1-((2-morpholinopyridin-4-yl)methyl)-3-(5-(4-(trifluoromethoxy)phenyl)-1,3,4-oxadiazol-2-yl)pyridin-2(1H)-one

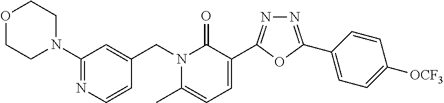 [SMILES: Cc1ccc(-c2nnc(-c3ccc(OC(F)(F)F)cc3)o2)c(=O)n1Cc1ccnc(N2CCOCC2)c1]

### 6-Methyl-1-((2-morpholinopyridin-4-yl)methyl)-3-(5-(4-(trifluoromethoxy)phenyl)-1,3,4-oxadiazol-2-yl)pyridin-2(1H)-one

Synthesized in an analogous method to Example 26: (2.8 mg, 6%). MS (ES+) C₂₅H₂₂F₃N₅O₄ requires: 513. found: 514 [M+H]+; ¹H NMR (600 MHz, CDCl₃) δ8.33 (d, J=7.55 Hz, 1H), 8.20 (d, J=8.03 Hz, 2H), 8.14 (d, J=5.2 Hz, 1H), 7.34 (d, J=8.03 Hz, 2H), 6.42 (d, J=5.2 Hz, 1H), 6.38 (s, 1H), 6.31 (d, J=7.55 Hz, 1H), 5.37 (s, 2H), 3.78 (t, J=5.0, 4H), 3.46 (t, J=5.0, 4H), 2.41 (s, 3H).

### EXAMPLE 28

### 1-(4-((6-Methyl-2-oxo-3-(5-(4-(trifluoromethoxy)phenyl)-1,3,4-oxadiazol-2-yl)pyridin-1(2H)-yl)methyl)pyridin-2-yl)piperidine-4-carbonitrile

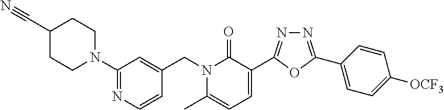 [SMILES: Cc1ccc(-c2nnc(-c3ccc(OC(F)(F)F)cc3)o2)c(=O)n1Cc1ccnc(N2CCC(C#N)CC2)c1]

### 1-(4-((6-Methyl-2-oxo-3-(5-(4-(trifluoromethoxy)phenyl)-1,3,4-oxadiazol-2-yl)pyridin-1(2H)-yl)methyl)pyridin-2-yl)piperidine-4-carbonitrile

Synthesized in an analogous method to Example 26: (1.8 mg, 4%). MS (ES+) C₂₇H₂₃F₃N₆O₃ requires: 536. found: 537 [M+H]+; ¹H NMR (600 MHz, CDCl₃) δ8.33 (d, J=7.5 Hz, 1H), 8.20 (d, J=8.8 Hz, 2H), 8.13 (d, J=5.2 Hz, 1H), 7.34 (d, J=8.8 Hz, 2H), 6.42 (s, 1H), 6.40 (d, J=5.2 Hz, 1H), 6.32 (d, J=7.5 Hz, 1H), 5.37 (s, 2H), 3.76 (m, 2H), 3.45 (m, 2H), 2.84 (m, 1H), 2.42 (s, 3H), 1.97 (m, 2H), 1.90 (m, 2H).

### EXAMPLE 29

### Methyl 2-{3-[(6-oxo-3-{3-[4-(trifluoromethoxy)phenyl]-1,2,4-oxadiazol-5-yl}-1,6-dihydropyridazin-1-yl)methyl]phenyl}acetate

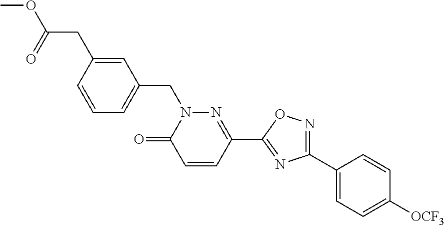 [SMILES: COC(=O)Cc1cccc(Cn2nc(-c3nc(-c4ccc(OC(F)(F)F)cc4)no3)ccc2=O)c1]

### Step 1

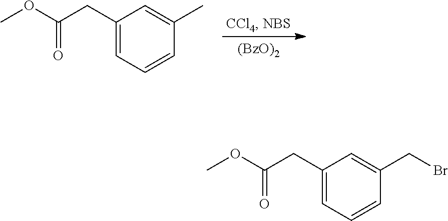

### Methyl 2-(3-(bromomethyl)phenyl)acetate

To a solution of methyl 3-methylphenylacetate (1.0 g, 6.0 mmol) in CCl₄ (6 mL) were added NBS (1.07 g, 6.0 mmol) and benzoyl peroxide (15 mg, 0.06 mmol). The mixture was heated to reflux at 90° C. for 6 h, then cooled to RT, filtered and concentrated under reduced pressure. The residue was purified by SiO₂ gel chromatography (2% to 10% Et₂O/Hexanes) to give methyl 2-(3-(bromomethyl)phenyl)acetate (808 mg, 55%). MS(ES⁺) C₁₀H₁₁BrO₂ requires: 243. found: 243/245 [M+H]⁺.

### Step 2

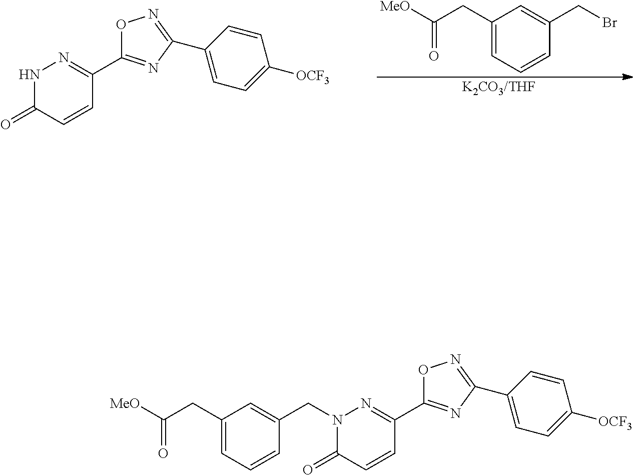

### Methyl 2-(3-((6-oxo-3-(3-(4-(trifluoromethoxy)phenyl)-1,2,4-oxadiazol-5-yl)pyridazin-1(6H)-yl)methyl)phenyl)acetate

Synthesized in an analogous manner to Example 24; MS(ES⁺) C₂₃H₁₇F₃N₄O₅ requires: 486. found: 487 [M+H]⁺; ¹H NMR (600 MHz, CDCl₃) δ 8.26 (d, J=8.4 Hz, 2H), 8.03 (d, J=9.6 Hz, 1H), 7.44 (s, 1H), 7.43 (d, J=7.8 Hz, 1H), 7.36 (d, J=8.4 Hz, 2H), 7.32 (m, 1H), 7.25 (d, J=7.8 Hz, 1H), 7.08 (d, J=9.6 Hz, 1H), 5.45 (s, 2H), 3.69 (s, 3H), 3.63 (s, 2H).

### EXAMPLE 30

### Methyl 2-methyl-2-{3-[(6-oxo-3-{3-[4-(trifluoromethoxy)phenyl]-1,2,4-oxadiazol-5-yl}-1,6-dihydropyridazin-1-yl)methyl]phenyl}propanoate

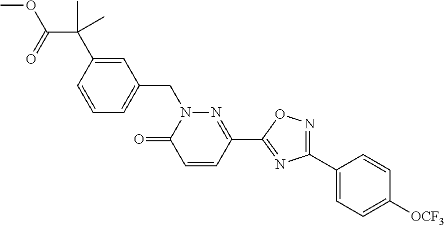 [SMILES: COC(=O)C(C)(C)c1cccc(Cn2nc(-c3nc(-c4ccc(OC(F)(F)F)cc4)no3)ccc2=O)c1]

### Steps 1 and 2

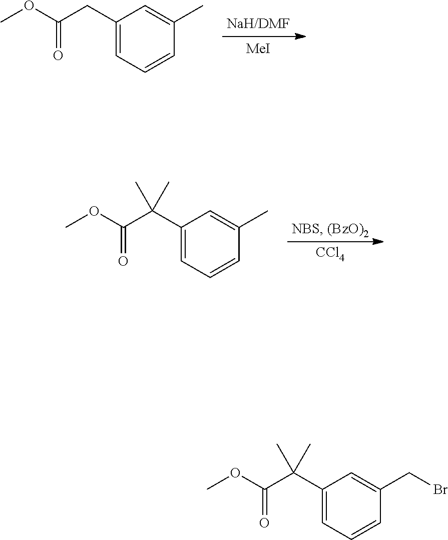

### Step 1: Methyl 2-methyl-2-(m-tolyl)propanoate

To a solution of methyl 2-(m-tolyl)acetate (1.0 g, 6.0 mmol) in DMF (15 mL) at 0° C. was added portionwise NaH (60% dispersion in mineral oil; 720 mg, 18.0 mmol). The resulting mixture was stirred at 0° C. for 1 h, and MeI (1.49 mL, 24.0 mmol) was added. The mixture was stirred at RT for 16 h, then poured into sat. aq. NH₄Cl and extracted with EtOAc. The organic layer was washed with brine, dried over Na₂SO₄, filtered, and concentrated under reduced pressure. The residue was purified by SiO₂ gel chromatography (1% to 5% EtOAc/Hexanes) to give methyl 2-methyl-2-(m-tolyl)propanoate (808 mg, 70%). MS(ES⁺) C₁₂H₁₆O₂ requires: 192. found: 193[M+H]⁺.

### Step 2: Methyl 2-(3-(bromomethyl)phenyl)-2-methylpropanoate

To a solution of methyl 2-methyl-2-(m-tolyl)propanoate (800 mg, 4.16 mmol) in CCl₄ (5 mL) were added NBS (741 mg, 4.16 mmol) and benzoyl peroxide (10 mg, 0.04 mmol). The mixture was heated to reflux at 90° C. for 6 h, cooled to RT, filtered and concentrated under reduced pressure. The residue was purified by SiO₂ gel chromatography (2% to 10% Et₂O/Hexanes) to give the title compound as a clear oil (505 mg, 45%). MS(ES⁺) C₁₂H₁₅BrO₂ requires: 271. found: 271/273[M+H]⁺.

### Step 3

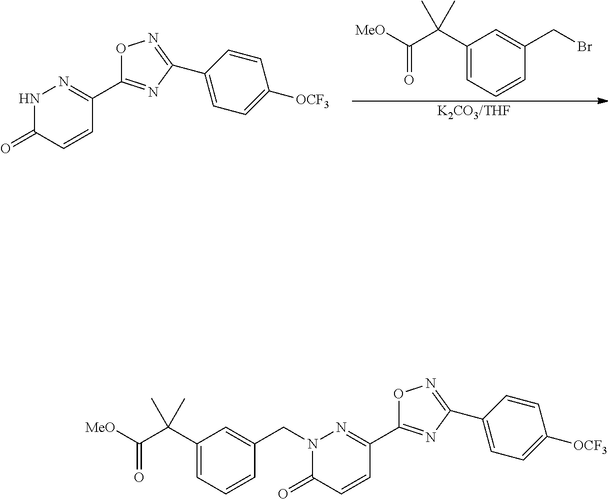

### Methyl 2-methyl-2-(3-((6-oxo-3-(3-(4-(trifluoromethoxy)phenyl)-1,2,4-oxadiazol-5-yl)pyridazin-1(6H)-yl)methyl)phenyl)propanoate

Synthesized in an analogous manner to Example 24; MS(ES⁺) C₂₅H₂₁F₃N₄O₅ requires: 514. found: 515 [M+H]⁺; ¹H NMR (600 MHz, CDCl₃) δ 8.20 (d, J=8.8 Hz, 2H), 8.02 (d, J=9.6 Hz, 1H), 7.53 (s, 1H), 7.39 (d, J=7.8 Hz, 1H), 7.36 (d, J=8.2 Hz, 2H), 7.31 (m, 1H), 7.29 (d, J=7.8 Hz, 1H), 7.08 (d, J=9.6 Hz, 1H), 5.46 (s, 2H), 3.65 (s, 3H), 1.58 (s, 6H).

### EXAMPLE 31

### 1-{[3-(Pyrrolidin-1-yl)phenyl]methyl}-5-{3-[4-(trifluoromethoxy)phenyl]-1,2,4-oxadiazol-5-yl}-1,2-dihydropyridin-2-one

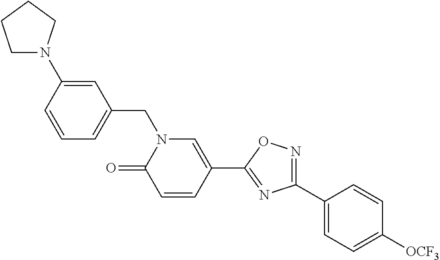 [SMILES: O=c1ccc(-c2nc(-c3ccc(OC(F)(F)F)cc3)no2)cn1Cc1cccc(N2CCCC2)c1]

### Step 1

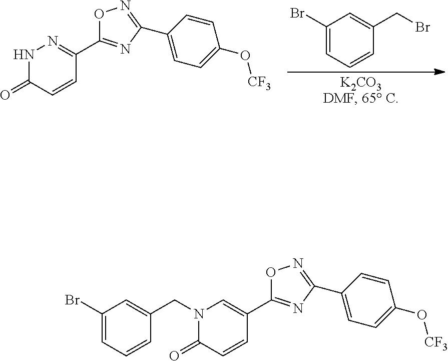

### 1-(3-Bromobenzyl)-5-(3-(4-(trifluoromethoxy)phenyl)-1,2,4-oxadiazol-5-yl)pyridin-2(1H)-one

To a suspension of 5-(3-(4-(trifluoromethoxy)phenyl)-1,2,4-oxadiazol-5-yl)pyridin-2(1H)-one (Example 7, Step 1; 200 mg, 0.619 mmol) and K₂CO₃ (128 mg, 0.928 mmol) in DMF (16 mL) was added 1-bromo-3-(bromomethyl)benzene (186 mg, 0.743 mmol). The mixture was stirred at RT for 1 h, diluted with water (5 mL) and extracted with EtOAc (3×5 mL). The combined organic layers were washed with water (4×5 mL) and brine (5 mL), dried over Na₂SO₄ and concentrated under reduced pressure. The brown solid residue was purified by SiO₂ gel chromatography (0% to 40% EtOAc/Hexanes) to provide the title compound as an off-white solid (220 mg, 72%): MS(ES⁺) C₂₁H₁₃BrF₃N₃O₃ requires: 492. found: 492/494 [M+H]⁺.

### Step 2

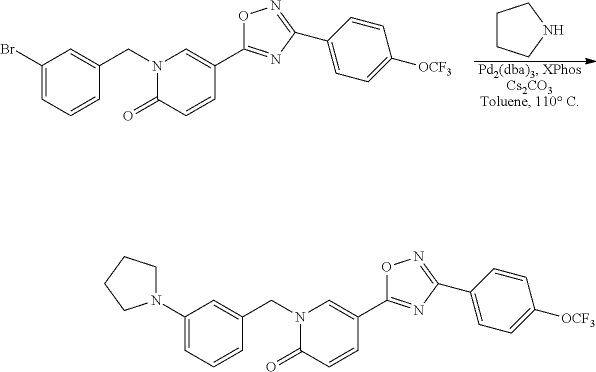

### 1-(3-(Pyrrolidin-1-yl)benzyl)-5-(3-(4-(trifluoromethoxy)phenyl)-1,2,4-oxadiazol-5-yl)pyridin-2(1H)-one

To a suspension of Cs₂CO₃ (36 mg, 0.11 mmol), XPhos (4.8 mg, 5.1 μmol), tris(dibenzylideneacetone)dipalladium(0) (4.7 mg, 6.09 μmol), and 1-(3-bromobenzyl)-5-(3-(4-(trifluoromethoxy)phenyl)-1,2,4-oxadiazol-5-yl)pyridin-2(1H)-one (25 mg, 0.051 mmol) in toluene (0.50 mL), previously degassed with N₂, was added pyrrolidine (3.6 mg, 0.051 mmol). The mixture was sealed in a vial and heated to 110° C. for 2 h, then cooled to RT, filtered through a pad of Celite and concentrated under reduced pressure. The residue was purified by prep-HPLC (Mobile phase: A=0.1% TFA/H₂O, B=0.1% TFA/MeCN; Gradient: B=30% to 70% in 12 min; Column: C18) to give the title compound; ¹H NMR (600 MHz, Chloroform-d) δ 8.38 (d, J=2.5 Hz, 1H), 8.16-8.13 (m, 2H), 8.05 (dd, J=9.5, 2.5 Hz, 1H), 7.34 (d, J=8.0 Hz, 2H), 7.30 (t, J=8.1 Hz, 1H), 6.83-6.79 (m, 3H), 6.76 (m, 1H), 5.21 (s, 2H), 3.42-3.35 (m, 4H), 2.11-2.02 (m, 4H); MS(ES⁺) C₂₅H₂₁F₃N₄O₃ requires: 482. found: 483 [M+H]⁺.

### EXAMPLE 32

### 1-[(3-Hydroxyphenyl)methyl]-5-{3-[4-(trifluoromethoxy)phenyl]-1,2,4-oxadiazol-5-yl}-1,2-dihydropyridin-2-one

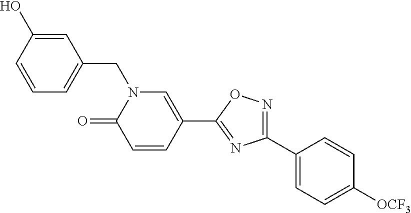 [SMILES: O=c1ccc(-c2nc(-c3ccc(OC(F)(F)F)cc3)no2)cn1Cc1cccc(O)c1]

### Step 1

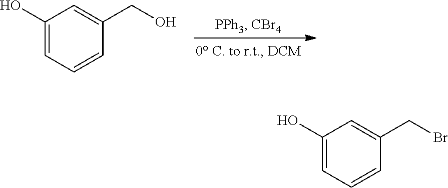 [SMILES: OCc1cccc(O)c1 ; Oc1cccc(CBr)c1]

### 3-(Bromomethyl)phenol

CBr₄ (4.01 g, 12.08 mmol) was added dropwise to a suspension of 3-hydroxybenzylalcohol (1.0 g, 8.06 mmol) and PPh₃ (3.17 g, 12.08 mmol) in DCM (40 mL) at 0° C. The reaction mixture was warmed to RT over 90 minutes and then concentrated under reduced pressure. The residue was purified by SiO₂ gel chromatography (5% to 50% EtOAc/Hexanes) to yield 3-(bromomethyl)phenol as a light brown crystalline solid (1.24 g, 82%).

### Step 2

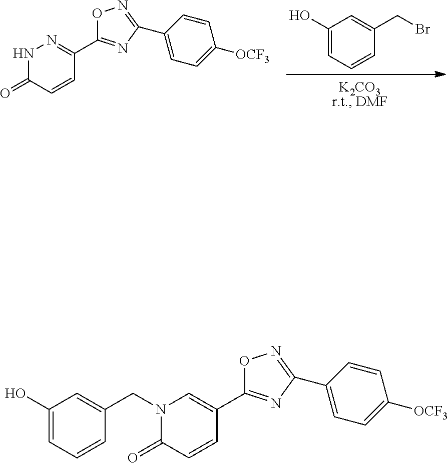

### 1-(3-Hydroxybenzyl)-5-(3-(4-(trifluoromethoxy)phenyl)-1,2,4-oxadiazol-5-yl)pyridin-2(1H)-one

Synthetized in an analogous manner to Example 26; 790 mg, 74%. MS(ES⁺) C₂₁H₁₄F₃N₃O₄ requires: 429. found 430 [M+H]⁺.

### EXAMPLE 33

### 1-{[3-(2-Methoxyethoxy)phenyl]methyl}-5-{3-[4-(trifluoromethoxy)phenyl]-1,2,4-oxadiazol-5-yl}-1,2-dihydropyridin-2-one

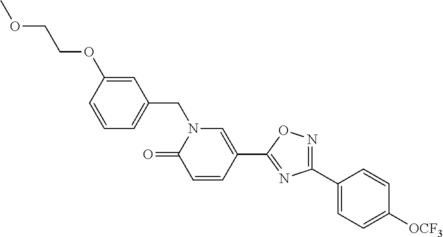 [SMILES: COCCOc1cccc(Cn2cc(-c3nc(-c4ccc(OC(F)(F)F)cc4)no3)ccc2=O)c1]

A mixture of K₂CO₃ (19.3 mg, 0.14 mmol), 1-bromo-2-methoxyethane (19.4 mg, 0.14 mmol) and 1-(3-hydroxybenzyl)-5-(3-(4-(trifluoromethoxy)phenyl)-1,2,4-oxadiazol-5-yl)pyridin-2(1H)-one (Example 32, 30.0 mg, 0.070 mmol) in DMF (0.35 mL) was stirred at RT for 16 h. The reaction was diluted with H₂O and extracted with 4:1 CHCl₃:iPrOH (3×). The combined organic layers were dried over MgSO₄ and concentrated under reduced pressure. The residue was dissolved in DMSO and purified by prep-HPLC to furnish the title compound; MS(ES⁺) C₂₄H₂₀F₃N₃O₅ requires: 487. found 488 [M+H]⁺; ¹H-NMR (600 MHz, CD₃OD) δ ppm 8.32 (d, J=2.5 Hz, 1H), 8.15 (d, J=8.8 Hz, 2H), 8.01 (d, J=9.6 Hz, 1H), 7.34 (d, J=8.2 Hz, 2H), 7.30 (m, 1H), 6.94 (m, 2H), 6.92 (m, 1H), 6.74 (d, J=9.7 Hz, 1H), 5.20 (s, 2H), 4.12 (s, 2H), 3.74 (s, 2H), 3.44 (s, 3H).

### EXAMPLE 34

### 1-{[3-(2-Aminoethoxy)phenyl]methyl}-5-{3-[4-(trifluoromethoxy)phenyl]-1,2,4-oxadiazol-5-yl}-1,2-dihydropyridin-2-one

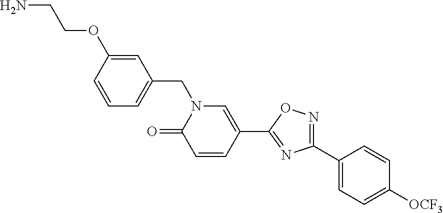 [SMILES: NCCOc1cccc(Cn2cc(-c3nc(-c4ccc(OC(F)(F)F)cc4)no3)ccc2=O)c1]

Acetyl chloride (34 uL, 0.47 mmol) was added dropwise to MeOH (0.16 mL) at 0° C., and the resulting mixture was stirred at 0° C. for 1 h. Tert-butyl (2-(3-((2-oxo-5-(3-(4-(trifluoromethoxy)phenyl)-1,2,4-oxadiazol-5-yl)pyridin-1 (2H)-yl)methyl)phenoxy)ethyl)carbamate (synthetized in an analogous manner to Example 33; 9.0 mg, 0.016 mmol) was then added in one portion, and the reaction mixture was stirred at RT for 16 h. The mixture was then concentrated under reduced pressure, the residue was dissolved in DMSO and purified by prep-HPLC to give the title compound; MS(ES⁺) C₂₃H₁₉F₃N₄O₄ requires: 472. found 473 [M+H]⁺; ¹H-NMR (600 MHz, CD₃OD) δ ppm 8.33 (d, J=2.5 Hz, 1H), 8.14 (d, J=8.8 Hz, 2H), 8.01 (dd, J=9.3 Hz, J=2.6 Hz, 1H), 7.34 (d, J=8.2 Hz, 2H), 7.30 (m, 1H), 6.92 (m, 3H), 6.76 (d, J=9.5 Hz, 1H), 5.21 (s, 2H), 3.99 (t, J=5.0 Hz, 2H), 3.08 (t, J=5.2 Hz, 2H).

### EXAMPLE 35

### N,N-Dimethyl-3-[(2-oxo-5-{3-[4-(trifluoromethoxy)phenyl]-1,2,4-oxadiazol-5-yl}-1,2-dihydropyridin-1-yl)methyl]benzene-1-sulfonamide

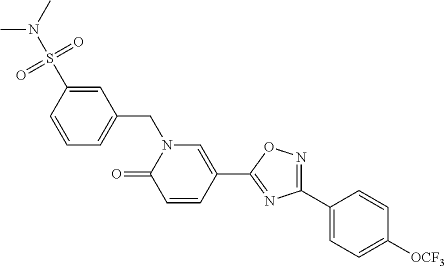 [SMILES: CN(C)S(=O)(=O)c1cccc(Cn2cc(-c3nc(-c4ccc(OC(F)(F)F)cc4)no3)ccc2=O)c1]

Sodium hydride (60% dispersion in mineral oil; 12.2 mg, 0.305 mmol) was added to a solution of 3-((2-oxo-5-(3-(4-(trifluoromethoxy)phenyl)-1,2,4-oxadiazol-5-yl)pyridin-1(2H)-yl)methyl)benzenesulfonamide (Example 119, synthetized in analogous manner to Example 26, 50 mg, 0.102 mmol) in DMF (1.0 ml). The reaction was stirred for 15 minutes and then iodomethane (0.019 ml, 0.305 mmol) was added. The reaction was stirred for 30 minutes at 45° C. and was then partitioned between H₂O (15 mL) and EtOAc (15 mL). The organic layer was separated, washed with H₂O (2×10 mL) and brine (5 mL), dried over Na₂SO₄, filtered, and concentrated under reduced pressure to afford the crude product, which was purified by SiO₂ gel chromatography (0% to 100% EtOAc/Hexanes) to give the title compound as a yellow solid. MS(ES⁺) C₂₃H₁₉F₃N₄O₅S requires: 520. found 521 [M+H]⁺; ¹H NMR (600 MHz, CDCl₃) δ 8.39 (d, J=2.3 Hz, 1H), 8.15 (d, J=8.7 Hz, 2H), 8.05 (dd, J=9.5, 2.3 Hz, 1H), 7.79 (s, 1H), 7.76 (d, J=7.6 Hz, 1H), 7.64-7.55 (m, 2H), 7.34 (d, J=8.3 Hz, 2H), 6.76 (d, J=9.5 Hz, 1H), 5.31 (s, 2H), 2.72 (s, 6H).

### EXAMPLE 36

### 2-Methyl-2-{3-[(2-oxo-5-{3-[4-(trifluoromethoxy)phenyl]-1,2,4-oxadiazol-5-yl}-1,2-dihydropyridin-1-yl)methyl]phenyl}propanoic acid

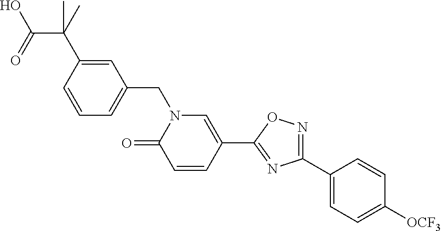 [SMILES: CC(C)(C(=O)O)c1cccc(Cn2cc(-c3nc(-c4ccc(OC(F)(F)F)cc4)no3)ccc2=O)c1]

Methyl 2-methyl-2-(3-((2-oxo-5-(3-(4-(trifluoromethoxy)phenyl)-1,2,4-oxadiazol-5-yl)pyridin-1(2H)-yl)methyl)phenyl)propanoate (synthetized in analogous manner to Example 30; 49 mg, 0.09 mmol) was treated with lithium hydroxide hydrate (38 mg, 0.9 mmol) in THF/MeOH/H₂O (3:1:1, 1 mL). The mixture was stirred at RT for 4 h and the volatiles were then removed under reduced pressure. The residue was diluted with water, the pH was adjusted to pH˜3 by addition of 1N aq. HCl, and the mixture was extracted with EtOAc. The organic layer was washed with brine, dried over Na₂SO₄, filtered, and concentrated under reduced pressure to give the title compound as an off-white solid; MS(ES⁺) C₂₅H₂₀F₃N₃O₅ requires: 499. found: 500 [M+H]+; ¹H NMR (600 MHz, CDCl₃) δ 8.36 (s, 1H), 8.12 (d, J=8.4 Hz, 2H), 7.99 (d, J=9.6 Hz, 1H), 7.45 (s, 1H), 7.40 (d, J=8.4 Hz, 1H), 7.35 (m, 1H), 7.32 (d, J=8.4 Hz, 2H), 7.22 (d, J=8.4 Hz, 1H), 6.74 (d, J=9.6 Hz, 1H), 5.24 (s, 2H), 1.60 (s, 6H).

### EXAMPLE 37

### 1-{[3-(propane-1-sulfonyl)phenyl]methyl}-5-{3-[4-(trifluoromethoxy)phenyl]-1,2,4-oxadiazol-5-yl}-1,2-dihydropyridin-2-one

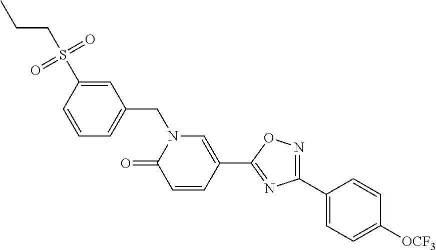 [SMILES: CCCS(=O)(=O)c1cccc(Cn2cc(-c3nc(-c4ccc(OC(F)(F)F)cc4)no3)ccc2=O)c1]

To a solution of 1-(3-(Methylsulfonyl)benzyl)-5-(3-(4-(trifluoromethoxy)phenyl)-1,2,4-oxadiazol-5-yl)pyridin-2(1H)-one (prepared in an analogous manner to Example 26, 50.0 mg, 0.102 mmol) in THF (1.00 ml) at −78° C. was added lithium diisopropylamide (2.0 M in THF; 0.061 ml, 0.122 mmol). The reaction was stirred for 15 minutes at −78° C., at 0° C. for 30 minutes and then cooled to −78° C. again. Bromoethane (0.030 ml, 0.407 mmol) was added and the reaction was allowed to warm to RT and stirred for further 3 h. The reaction mixture was then concentrated under reduced pressure and the residue was purified by prep-HPLC (Mobile phase: A=0.01% TFA/H₂O, B=0.01% TFA/MeCN; Gradient: B=50%-90% in 12 min; Column: C18) to afford the title compound as a yellow solid. MS (ES⁺) C₂₄H₂₀F₃N₃O₅S requires: 519. found 520 [M+H]⁺; ¹H NMR (600 MHz, CDCl₃) δ 8.40 (d, J=2.5 Hz, 1H), 8.19-8.14 (m, 2H), 8.06 (dd, J=9.7, 2.5 Hz, 1H), 7.91 (s, 1H), 7.89 (d, J=7.9 Hz, 1H), 7.66 (d, J=9.5 Hz, 1H), 7.63-7.57 (m, 1H), 7.34 (d, J=8.3 Hz, 2H), 6.77 (d, J=9.5 Hz, 1H), 5.32 (s, 2H), 3.11-3.05 (m, 2H), 1.81-1.72 (m, 2H), 1.00 (t, J=7.4 Hz, 3H).

### EXAMPLE 38

### 1-{[3-(1-Hydroxy-2-methylpropan-2-yl)phenyl]methyl}-5-{3-[4-(trifluoromethoxy)phenyl]-1,2,4-oxadiazol-5-yl}-1,2-dihydropyridin-2-one

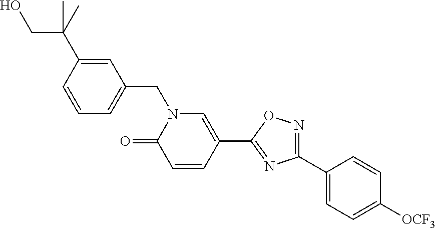 [SMILES: CC(C)(CO)c1cccc(Cn2cc(-c3nc(-c4ccc(OC(F)(F)F)cc4)no3)ccc2=O)c1]

### Steps 1 to 2

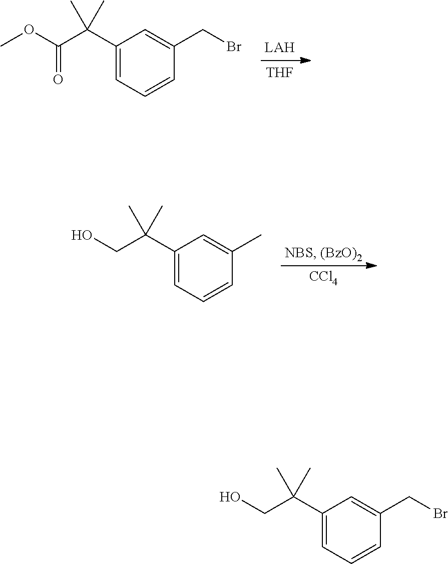

### Step 1: 2-Methyl-2-(m-tolyl)propan-1-ol

To a solution of methyl 2-(3-(bromomethyl)phenyl)-2-methylpropanoate (Example 30, Step 2; 500 mg, 1.84 mmol) in ether (10 mL) at 0° C. was added dropwise lithium aluminum hydride (1M in THF, 0.92 mL, 0.92 mmol). The mixture was stirred at 0° C. for 30 minutes, at RT for 1 h, then cooled to 0° C. again and a further portion of lithium aluminum hydride (1M in THF, 0.92 mL, 0.92 mmol) was added dropwise. The reaction mixture was stirred at 0° C. for 30 minutes and at RT for 1 h. H₂O (50 μL) was added slowly at 0° C. with stirring, followed by 1N aq. NaOH (50 uL) and MeOH (100 μL). The mixture was filtered through a pad of Celite and concentrated under reduced pressure. The residue was purified by SiO₂ gel chromatography (5% to 50% Et₂O/Hexanes) to give 2-methyl-2-(m-tolyl)propan-1-ol as a clear oil (240 mg, 79%). MS(ES⁺) C₁₁H₁₆O requires: 164. found: 165 [M+H]⁺

### Step 2: 2-(3-(Bromomethyl)phenyl)-2-methylpropan-1-ol

Synthesized in an analogous manner to Example 30, Step 2; 207 mg, 64%. MS(ES⁺) C₁₁H₁₅BrO requires: 243. found: 243/245 [M+H]⁺.

### Step 3

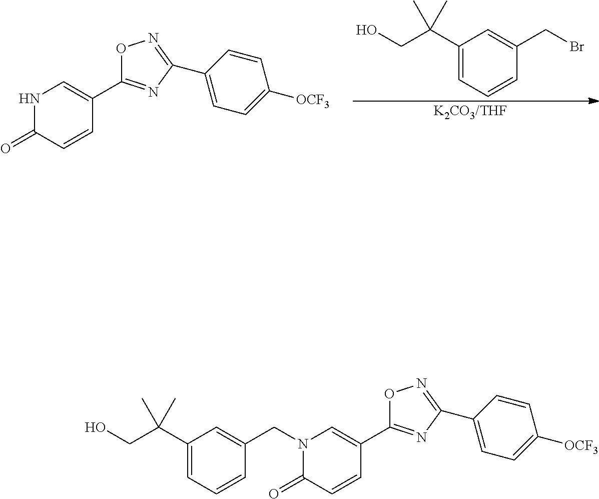

### 1-(3-(1-Hydroxy-2-methylpropan-2-yl)benzyl)-5-(3-(4-(trifluoromethoxy)phenyl)-1,2,4-oxadiazol-5-yl)pyridin-2(1H)-one

Synthesized in an analogous manner to Example 26; MS(ES⁺) C₂₅H₂₂F₃N₃O₄ requires: 485. found: 486 [M+H]⁺; ¹H NMR (600 MHz, CDCl₃) δ 8.36 (s, 1H), 8.13 (d, J=8.4 Hz, 2H), 8.01 (d, J=9.6 Hz, 1H), 7.44 (s, 1H), 7.38 (m, 1H), 7.36 (d, J=8.4 Hz, 1H), 7.33 (d, J=8.4 Hz, 2H), 7.18 (d, J=8.4 Hz, 1H), 6.73 (d, J=9.6 Hz, 1H), 5.24 (s, 2H), 3.63 (s, 2H), 1.34 (s, 6H).

### EXAMPLE 39

### 1-{[3-(2-Hydroxypropan-2-yl)phenyl]methyl}-5-{3-[4-(trifluoromethoxy)phenyl]-1,2,4-oxadiazol-5-yl}-1,2-dihydropyridin-2-one

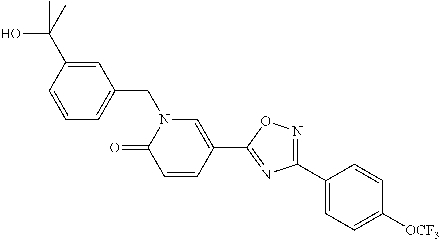 [SMILES: CC(C)(O)c1cccc(Cn2cc(-c3nc(-c4ccc(OC(F)(F)F)cc4)no3)ccc2=O)c1]

### Step 1

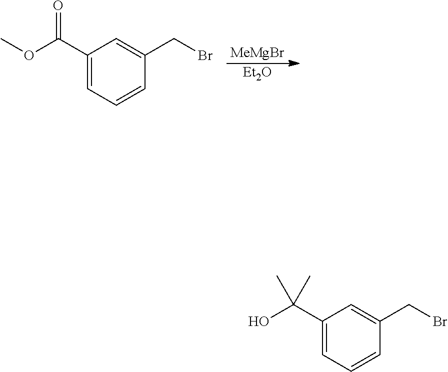

### 2-(3-(Bromomethyl)phenyl)propan-2-ol

To a solution of methyl 3-(bromomethyl)benzoate (0.5 g, 2.18 mmol) in ether (10 mL), at 0° C. was added dropwise methylmagnesium bromide (3M in Et₂O, 2.18 mL, 6.55 mmol). The reaction mixture was warmed up to 45° C. and stirred for 2 h, and then poured into an ice-cooled saturated aq. solution of NH₄Cl and extracted with EtOAc. The organic layer was washed with brine, dried over Na₂SO₄, filtered, and concentrated under reduced pressure. The residue was purified by SiO₂ gel chromatography (8% to 60% EtOAc/Hexanes) to give 2-(3-(bromomethyl)phenyl)propan-2-ol as a clear oil (202 mg, 40%). MS(ES⁺) C₁₀H₁₃BrO requires: 229. found: 229/231 [M+H]⁺.

### Step 2

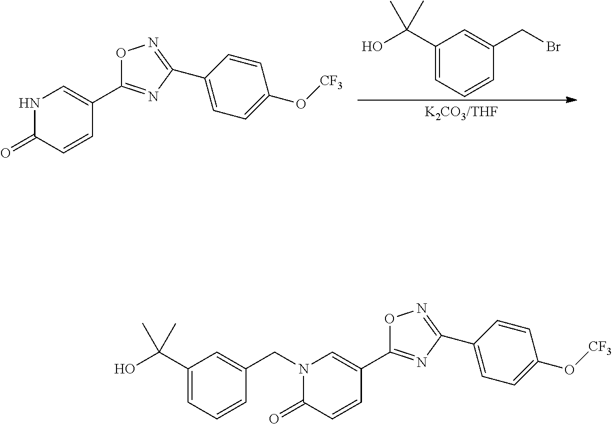 [SMILES: O=c1ccc(-c2nc(-c3ccc(OC(F)(F)F)cc3)no2)c[nH]1 ; CC(C)(O)c1cccc(CBr)c1 ; CC(C)(O)c1cccc(Cn2cc(-c3nc(-c4ccc(OC(F)(F)F)cc4)no3)ccc2=O)c1]

### 1-(3-(2-Hydroxypropan-2-yl)benzyl)-5-(3-(4-(trifluoromethoxy)phenyl)-1,2,4-oxadiazol-5-yl)pyridin-2(1H)-one

Synthesized in an analogous manner to Example 26; MS(ES⁺) C₂₄H₂₀F₃N₃O₄ requires: 471. found: 472 [M+H]⁺; ¹H NMR (600 MHz, CDCl₃) δ 8.35 (s, 1H), 8.13 (d, J=8.4 Hz, 2H), 8.01 (d, J=9.6 Hz, 1H), 7.56 (s, 1H), 7.45 (d, J=8.4 Hz, 1H), 7.36 (d, J=8.4 Hz, 1H), 7.33 (d, J=8.4 Hz, 2H), 7.22 (d, J=8.4 Hz, 1H), 6.73 (d, J=9.6 Hz, 1H), 5.25 (s, 2H), 1.59 (s, 6H).

### EXAMPLE 40

### (2E)-N,N-dimethyl-3-{3-[(2-oxo-5-{3-[4-(trifluoromethoxy)phenyl]-1,2,4-oxadiazol-5-yl}-1,2-dihydropyridin-1-yl)methyl]phenyl}prop-2-enamide

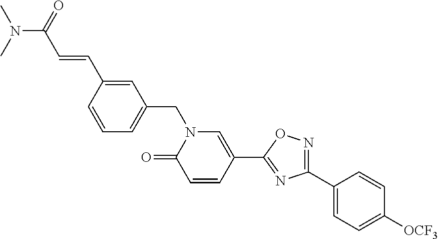 [SMILES: CN(C)C(=O)/C=C/c1cccc(Cn2cc(-c3nc(-c4ccc(OC(F)(F)F)cc4)no3)ccc2=O)c1]

To a solution of 1-(3-bromobenzyl)-5-(3-(4-(trifluoromethoxy)phenyl)-1,2,4-oxadiazol-5-yl)pyridin-2(1H)-one (Example 31, Step 1; 100 mg, 0.20 mmol) in DMF (2 mL) were added N,N-dimethylacrylamide (101 mg, 1.0 mmol), Pd(Oac)₂ (4.6 mg, 0.02 mmol), PPh₃(10.7 mg, 0.04 mmol), and K₂CO₃ (56 mg, 0.40 mmol). The mixture was heated under N2 in a sealed vial at 120° C. for 20 h. The mixture was cooled to RT, diluted with water and extracted with EtOAc. The organic layer was dried over Na₂SO₄, filtered, and concentrated under reduced pressure. The residue was purified by SiO₂ gel chromatography (0% to 5% MeOH/EtOAc) to give the title compound; MS(ES⁺) C₂₆H₂₁F₃N₄O₄ requires: 510. found: 511 [M+H]⁺; ¹H NMR (600 MHz, CDCl₃) δ 8.37 (s, 1H), 8.15 (d, J=8.4 Hz, 2H), 8.03 (d, J=9.6 Hz, 1H), 7.64 (d, J=15.5 Hz, 1H), 7.52 (s, 1H), 7.51 (d, J=8.4 Hz, 2H), 7.39 (m, 1H), 7.35 (m, 2H), 6.90 (d, J=15.5 Hz, 1H), 6.76 (d, J=9.6 Hz, 1H), 5.26 (s, 2H), 3.18 (s, 3H), 3.07 (s, 3H).

### EXAMPLE 41

### 1-{[3-(4-Methanesulfonylpiperidin-1-yl)phenyl]methyl}-5-{3-[4-(trifluoromethoxy)phenyl]-1,2,4-oxadiazol-5-yl}-1,2-dihydropyridin-2-one

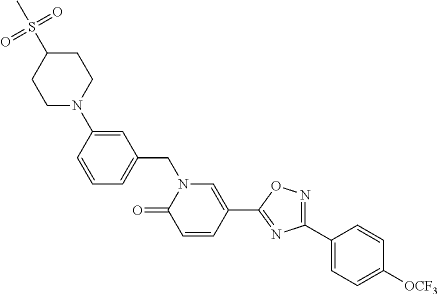 [SMILES: CS(=O)(=O)C1CCN(c2cccc(Cn3cc(-c4nc(-c5ccc(OC(F)(F)F)cc5)no4)ccc3=O)c2)CC1]

Synthesized in an analogous manner to Example 31; MS(ES⁺) C₂₇H₂₅F₃N₄O₅S requires: 574. found: 575 [M+H]⁺; ¹H NMR (600 MHz, CDCl₃) δ 8.35 (s, 1H), 8.14 (d, J=8.4 Hz, 2H), 8.03 (d, J=9.6 Hz, 1H), 7.34 (d, J=8.4 Hz, 2H), 7.31 (m, 1H), 7.04 (s, 1H), 7.97 (dd, J=8.4 Hz, 2.4 Hz, 1H), 6.91 (d, J=8.4 Hz, 1H), 6.77 (d, J=9.6 Hz, 1H), 5.20 (s, 2H), 3.86 (m, 2H), 2.99 (m, 1H), 2.87 (s, 3H), 2.85 (m, 2H), 2.29 (m, 2H), 2.02 (m, 2H).

### EXAMPLE 42

### 1-{[3-(2-Hydroxyethyl)phenyl]methyl}-5-{3-[4-(trifluoromethoxy)phenyl]-1,2,4-oxadiazol-5-yl}-1,2-dihydropyridin-2-one

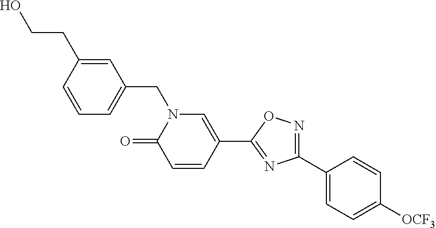 [SMILES: O=c1ccc(-c2nc(-c3ccc(OC(F)(F)F)cc3)no2)cn1Cc1cccc(CCO)c1]

### Steps 1 to 2:

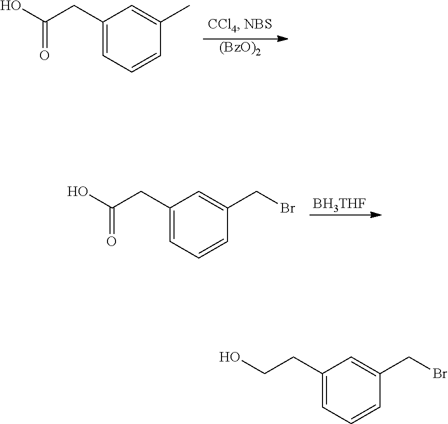

### Step 1: 2-(3-(Bromomethyl)phenyl)acetic acid

Synthesized in an analogous manner to Example 30, Step 2; 178 mg, 23%. MS(ES⁺) C₉H₉BrO₂ requires: 229. found: 229/231 [M+H]⁺

### Step 2: 2-(3-(Bromomethyl)phenyl)ethanol

To a solution of 2-(3-(bromomethyl)phenyl)acetic acid (75 mg, 0.327 mmol) in THF (1 mL) at 0° C. was added BH₃.THF (1M in THF, 0.426 mL, 0.426 mmol) dropwise. The mixture was stirred at 0° C. for 1 h and at RT for 12 h, then diluted with THF/H₂O (1:1 v:v, 2 mL) and washed with saturated aq. K₂CO₃. The phases were separated, the aqueous layer was extracted with THF (2×), the combined organic layers were dried (Na₂SO₄) and concentrated under reduced pressure. The residue was purified by SiO₂ gel chromatography (10% to 100% EtOAc/Hexanes) to give 2-(3-(bromomethyl)phenyl)ethanol (50 mg, 71%). MS(ES⁺) C₉H₁₁BrO requires: 215. found: 215/217 [M+H]⁺.

### Step 3

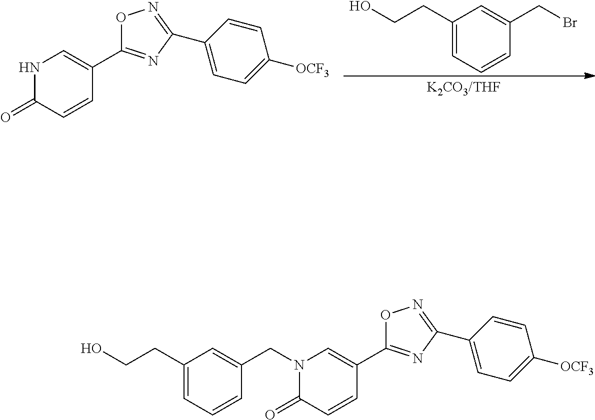 [SMILES: O=c1ccc(-c2nc(-c3ccc(OC(F)(F)F)cc3)no2)c[nH]1 ; OCCc1cccc(CBr)c1 ; O=c1ccc(-c2nc(-c3ccc(OC(F)(F)F)cc3)no2)cn1Cc1cccc(CCO)c1]

### 1-(3-(2-Hydroxyethyl)benzyl)-5-(3-(4-(trifluoromethoxy)phenyl)-1,2,4-oxadiazol-5-yl)pyridin-2(1H)-one

Synthesized in an analogous manner to Example 26; MS(ES⁺) C₂₃H₁₈F₃N₃O₄ requires: 457. found: 458 [M+H]⁺; ¹H NMR (600 MHz, CDCl₃) δ 8.35 (s, 1H), 8.14 (d, J=7.2 Hz, 2H), 8.01 (d, J=8.4 Hz, 1H), 7.34 (m, 3H), 7.26 (s, 1H), 7.22 (d, J=7.2 Hz, 2H), 6.74 (d, J=8.4 Hz, 1H), 5.23 (s, 2H), 3.87 (t, J=6.0 Hz, 2H), 2.88 (t, J=6.0 Hz, 2H).

### EXAMPLE 43

### 1-{[3-(Hydroxymethyl)phenyl]methyl}-5-{3-[4-(trifluoromethoxy)phenyl]-1,2,4-oxadiazol-5-yl}-1,2-dihydropyridin-2-one

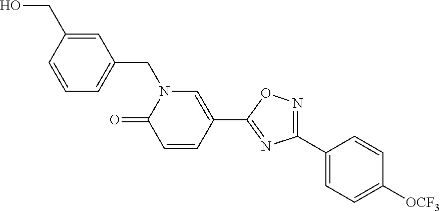 [SMILES: O=c1ccc(-c2nc(-c3ccc(OC(F)(F)F)cc3)no2)cn1Cc1cccc(CO)c1]

A mixture of 1-(3-(bromomethyl)benzyl)-5-(3-(4-(trifluoromethoxy)phenyl)-1,2,4-oxadiazol-5-yl)pyridin-2(1H)-one (prepared in analogous manner to Example 26, 40 mg, 0.079 mmol) and Cs₂CO₃ (77 mg, 0.237 mmol) in dioxane/water (1:1 v:v, 1 mL) was heated at 110° C. for 12 h. After cooling to RT the mixture was acidified with conc. HCl and then extracted with EtOAc. The organic layer was washed with brine, dried over Na₂SO₄, filtered, concentrated, and purified by SiO₂ gel chromatography (20% to 100% EtOAc/Hexanes) to give the title compound; MS(ES⁺) C₂₂H₁₆F₃N₃O₄ requires: 443. found: 444 [M+H]⁺; ¹H NMR (600 MHz, CDCl₃) δ 8.36 (s, 1H), 8.14 (d, J=8.4 Hz, 2H), 8.01 (d, J=9.6 Hz, 1H), 7.39 (m, 2H), 7.34 (m, 3H), 7.29 (d, J=8.4 Hz, 1H), 6.74 (d, J=9.6 Hz, 1H), 5.24 (s, 2H), 4.71 (s, 2H).

### EXAMPLE 44

### 1-({3-[(Acetylsulfanyl)methyl]phenyl}methyl)-5-{3-[4-(trifluoromethoxy)phenyl]-1,2,4-oxadiazol-5-yl}-1,2-dihydropyridin-2-one

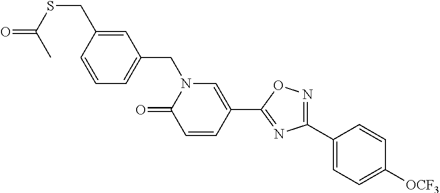 [SMILES: CC(=O)SCc1cccc(Cn2cc(-c3nc(-c4ccc(OC(F)(F)F)cc4)no3)ccc2=O)c1]

To a solution of 1-(3-(bromomethyl)benzyl)-5-(3-(4-(trifluoromethoxy)phenyl)-1,2,4-oxadiazol-5-yl)pyridin-2(1H)-one (prepared in analogous manner to Example 26, 54 mg, 0.11 mmol) in acetone (0.53 ml) was added potassium thioacetate (18 mg, 0.16 mmol). The mixture was stirred for 2 h at RT, filtered and concentrated under reduced pressure. The residue was purified by SiO₂ gel chromatography (10% to 40% EtOAc/Hexanes) to give the title compound as a white solid; ¹H NMR (600 MHz, Chloroform-d) δ 8.33 (d, J=2.5 Hz, 1H), 8.15 (d, J=8.7 Hz, 2H), 8.02 (dd, J=9.6, 2.4 Hz, 1H), 7.36-7.26 (m, 5H), 7.23 (d, J=7.8 Hz, 1H), 6.75 (d, J=9.4 Hz, 1H), 5.21 (s, 2H), 4.11 (s, 2H), 2.34 (s, 3H); MS(ES⁺) C₂₄H₁₈F₃N₃O₄S requires: 501. found: 502 [M+H]⁺.

### EXAMPLE 45

### 1-{[3-(Sulfanylmethyl)phenyl]methyl}-5-{3-[4-(trifluoromethoxy)phenyl]-1,2,4-oxadiazol-5-yl}-1,2-dihydropyridin-2-one

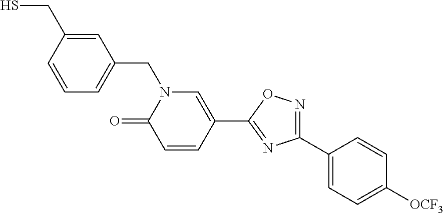 [SMILES: O=c1ccc(-c2nc(-c3ccc(OC(F)(F)F)cc3)no2)cn1Cc1cccc(CS)c1]

To a solution of S-(3-((2-oxo-5-(3-(4-(trifluoromethoxy)phenyl)-1,2,4-oxadiazol-5-yl)pyridin-1(2H)-yl)methyl)benzyl)ethanethioate (Example 44, 40 mg, 0.080 mmol) in MeOH (0.80 mL) was added K₂CO₃ (33 mg, 0.24 mmol). The mixture was stirred at RT for 12 h, diluted with 1 N aq. HCl (1 mL) and extracted with EtOAc (3×2 mL). The organic layer was washed with brine (2 mL), dried over Na₂SO₄, filtered and concentrated under reduced pressure. The residue was purified by SiO₂ gel chromatography (10% to 40% EtOAc/Hexanes) to the title compound as a white solid; MS(ES⁺) C₂₂H₁₆F₃N₃O₃S requires: 459. found: 460 [M+H]⁺; ¹H NMR (600 MHz, Chloroform-d) δ 8.35 (d, J=2.4 Hz, 1H), 8.15 (d, J=8.7 Hz, 2H), 8.02 (dd, J=9.6, 2.4 Hz, 1H), 7.38-7.30 (m, 5H), 7.26-7.22 (m, 1H), 6.75 (d, J=9.6 Hz, 1H), 5.23 (s, 2H), 3.75 (d, J=7.7 Hz, 2H), 1.78 (t, J=7.7 Hz, 1H).

### EXAMPLE 46

### Methyl 3-{3-[(2-oxo-5-{3-[4-(trifluoromethoxy)phenyl]-1,2,4-oxadiazol-5-yl}-1,2-dihydropyridin-1-yl)methyl]phenyl}propanoate

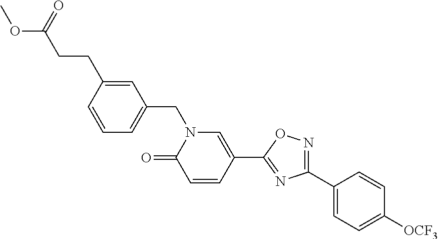 [SMILES: COC(=O)CCc1cccc(Cn2cc(-c3nc(-c4ccc(OC(F)(F)F)cc4)no3)ccc2=O)c1]

### Steps 1 to 3

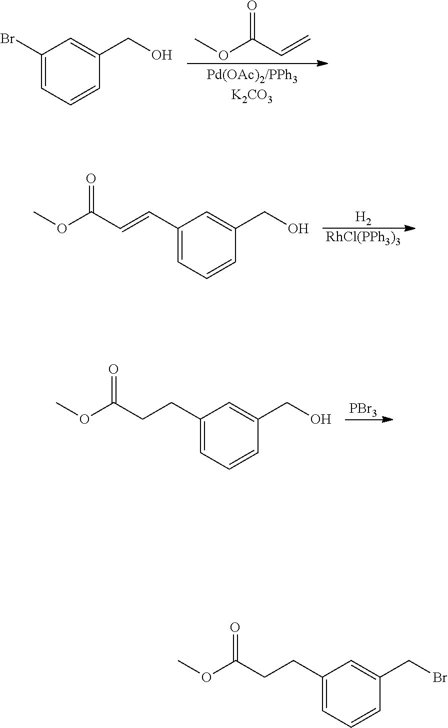

### Step 1: (E)-Methyl 3-(3-(hydroxymethyl)phenyl)acrylate

Synthesized in an analogous manner to Example 40; 802 mg, 39%. MS(ES⁺) C₁₁H₁₂O₃ requires: 192. found: 193 [M+H]⁺.

### Step 2: Methyl 3-(3-(hydroxymethyl)phenyl)propanoate

A mixture of (E)-methyl 3-(3-(hydroxymethyl)phenyl)acrylate (200 mg, 1.04 mmol) and (Ph3P)₃RhCl (96 mg, 0.1 mmol) in EtOH (5 mL) was stirred under H2 (50 PSI) at RT for 20 h, then filtered through a pad of Celite and concentrated under reduced pressure. The residue was purified by SiO₂ gel chromatography (20% to 100% EtOAc/Hexanes) to give methyl 3-(3-(hydroxymethyl)phenyl)propanoate (105 mg, 52%). MS(ES⁺) C₁₁H₁₄O₃ requires: 194. found: 195 [M+H]⁺.

### Step 3: Methyl 3-(3-(bromomethyl)phenyl)propanoate

To a solution of methyl 3-(3-(hydroxymethyl)phenyl)propanoate (100 mg, 0.515 mmol) in DCM (2 mL) was added dropwise PBr₃ (0.058 mL, 0.618 mmol), followed by a catalytic amount of pyridine (0.05 ml). The mixture was stirred at RT for 2 h and then poured onto ice. The organic layer was separated, dried over Na₂SO₄, filtered, and concentrated under reduced pressure. The residue was purified by SiO₂ gel chromatography (0% to 20% EtOAc/Hexanes) to give methyl 3-(3-(bromomethyl)phenyl)propanoate (53 mg, 40%). MS(ES⁺) C₁₁H₁₃BrO₂ requires: 257. found: 257/259 [M+H]⁺.

### Step 4

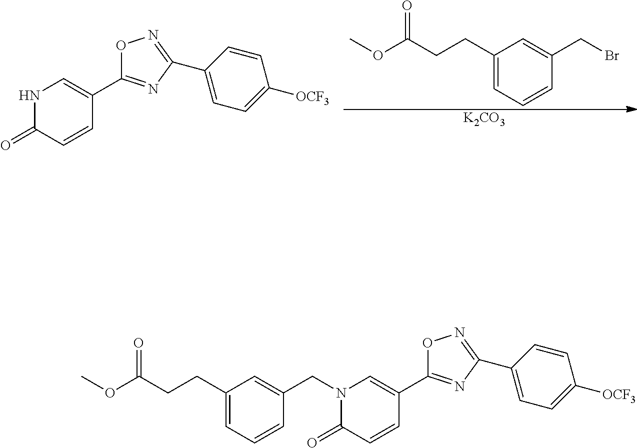 [SMILES: O=c1ccc(-c2nc(-c3ccc(OC(F)(F)F)cc3)no2)c[nH]1 ; COC(=O)CCc1cccc(CBr)c1 ; "K2CO3" ; COC(=O)CCc1cccc(Cn2cc(-c3nc(-c4ccc(OC(F)(F)F)cc4)no3)ccc2=O)c1]

### Methyl 3-(3-((2-oxo-5-(3-(4-(trifluoromethoxy)phenyl)-1,2,4-oxadiazol-5-yl)pyridin-1(2H)-yl)methyl)phenyl)propanoate

Synthesized in an analogous manner to Example 26; MS(ES⁺) C₂₅H₂₀F₃N₃O₅ requires: 499. found: 500 [M+H]⁺; ¹H NMR (600 MHz, CDCl₃) δ 8.34 (s, 1H), 8.14 (d, J=8.4 Hz, 2H), 8.01 (d, J=9.6 Hz, 1H), 7.33 (m, 3H), 7.20 (m, 3H), 6.74 (d, J=9.6 Hz, 1H), 5.22 (s, 2H), 3.65 (s, 3H), 2.96 (t, J=7.8 Hz, 2H), 2.63 (t, J=7.8 Hz, 2H).

### EXAMPLE 47

### 3-{3-[(2-oxo-5-{3-[4-(trifluoromethoxy)phenyl]-1,2,4-oxadiazol-5-yl}-1,2-dihydropyridin-1-yl)methyl]phenyl}propanoic acid

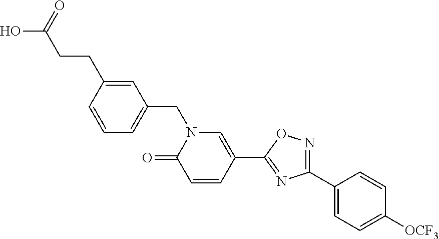 [SMILES: O=C(O)CCc1cccc(Cn2cc(-c3nc(-c4ccc(OC(F)(F)F)cc4)no3)ccc2=O)c1]

To a solution of methyl 3-(3-((2-oxo-5-(3-(4-(trifluoromethoxy)phenyl)-1,2,4-oxadiazol-5-yl)pyridin-1(2H)-yl)methyl)phenyl)propanoate (Example 46, 50 mg, 0.10 mmol) in THF/MeOH/H₂O (3:1:1 v:v:v, 2.5 mL) was added LiOH.H₂O (48 mg, 2.0 mmol). The mixture was stirred at RT for 4 h, and the volatiles were removed under reduced pressure. The residue was diluted with water, the pH was adjusted to pH ˜3 by addition of 1N aq. HCl, and the mixture was extracted with EtOAc. The organic layer was washed with brine, dried over Na₂SO₄, filtered, and concentrated under reduced pressure to give the title compound; MS(ES⁺) C₂₄H₁₈F₃N₃O₅ requires: 485. found: 486 [M+H]⁺; ¹H NMR (600 MHz, DMSO-d₆) δ 12.12 (bs, 1H), 8.98 (s, 1H), 8.18 (d, J=8.4 Hz, 2H), 8.06 (d, J=9.6 Hz, 1H), 7.59 (d, J=8.4 Hz, 2H), 7.27 (m, 2H), 7.17 (d, J=8.4 Hz, 2H), 6.65 (d, J=9.6 Hz, 1H), 5.25 (s, 2H), 2.81 (t, J=7.8 Hz, 2H), 2.52 (t, J=7.8 Hz, 2H).

### EXAMPLE 48

### 1-{[3-(3-Hydroxypropyl)phenyl]methyl}-5-{3-[4-(trifluoromethoxy)phenyl]-1,2,4-oxadiazol-5-yl}-1,2-dihydropyridin-2-one

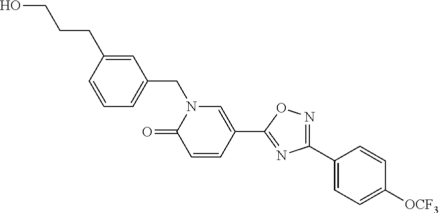 [SMILES: O=c1ccc(-c2nc(-c3ccc(OC(F)(F)F)cc3)no2)cn1Cc1cccc(CCCO)c1]

To a solution of 3-(3-((2-oxo-5-(3-(4-(trifluoromethoxy)phenyl)-1,2,4-oxadiazol-5-yl)pyridin-1(2H)-yl)methyl)phenyl)propanoic acid (Example 47; 20 mg, 0.041 mmol) in THF (1 mL) at 0° C. was added dropwise BH3.THF (1M in THF, 0.054 mL, 0.054 mmol). The mixture was stirred at 0° C. for 1 h and at RT for 12 h, the diluted with THF/H₂O (1:1 v:v, 2 mL) and washed with saturated aq. K₂CO₃. The phases were separated, the aqueous layer was extracted with THF (2×), the combined organic layers were dried (Na₂SO₄) and concentrated under reduced pressure. The residue was purified by SiO₂ gel chromatography (20% to 100% EtOAc/Hexanes; then 0% to 10% MeOH/EtOAc) to give the title compound; MS(ES⁺) C₂₄H₂₀F₃N₃O₄ requires: 471. found: 472 [M+H]⁺; ¹H NMR (600 MHz, DMSO-d₆) δ 8.98 (s, 1H), 8.18 (d, J=8.4 Hz, 2H), 8.06 (d, J=9.6 Hz, 1H), 7.59 (d, J=8.4 Hz, 2H), 7.26 (m, 2H), 7.14 (m, 2H), 6.65 (d, J=9.6 Hz, 1H), 5.26 (s, 2H), 4.46 (m, 1H), 3.40 (m, 2H), 2.59 (m, 2H), 1.70 (m, 2H).

### EXAMPLE 49

### N,N-Dimethyl-3-{3-[(2-oxo-5-{3-[4-(trifluoromethoxy)phenyl]-1,2,4-oxadiazol-5-yl}-1,2-dihydropyridin-1-yl)methyl]phenyl}propanamide

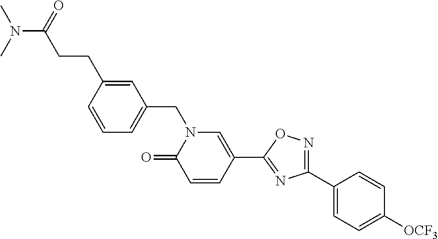 [SMILES: CN(C)C(=O)CCc1cccc(Cn2cc(-c3nc(-c4ccc(OC(F)(F)F)cc4)no3)ccc2=O)c1]

To a solution of 3-(3-((2-oxo-5-(3-(4-(trifluoromethoxy)phenyl)-1,2,4-oxadiazol-5-yl)pyridin-1(2H)-yl)methyl)phenyl)propanoic acid (Example 47, 20 mg, 0.041 mmol) in THF (1 mL) were added a catalytic amount of DMF (0.05 ml) followed by oxalyl chloride (7.84 mg, 0.062 mmol) dropwise. The mixture was stirred at 50° C. for 3 h and then concentrated under reduced pressure. The residue was dissolved in pyridine (0.5 mL), dimethylamine (1M solution in THF, 0.062 mL, 0.062 mmol) was added and the mixture was stirred at RT for further 16 h. The mixture was then partitioned between water and EtOAc, the organic layer was washed with brine, dried over Na₂SO₄, filtered and concentrated under reduced pressure. The residue was purified by SiO₂ gel chromatography (10% to 100% EtOAc/Hexanes; then 0% to 20% MeOH/EtOAc) to give the title compound; MS(ES⁺) C₂₆H₂₃F₃N₄O₄ requires: 512. found: 513 [M+H]⁺; ¹H NMR (600 MHz, DMSO-d₆) δ 8.98 (s, 1H), 8.18 (d, J=8.4 Hz, 2H), 8.06 (d, J=9.6 Hz, 1H), 7.59 (d, J=8.4 Hz, 2H), 7.27 (m, 1H), 7.23 (m, 1H), 7.15 (m, 2H), 6.65 (d, J=9.6 Hz, 1H), 5.25 (s, 2H), 2.89 (s, 3H), 2.78 (s, 3H), 1.40 (m, 2H), 1.32 (m, 2H).

### EXAMPLE 50

### 1-{[2-(2-Hydroxypropan-2-yl)pyridin-4-yl]methyl}-5-{3-[4-(trifluoromethoxy)phenyl]-1,2,4-oxadiazol-5-yl}-1,2-dihydropyridin-2-one

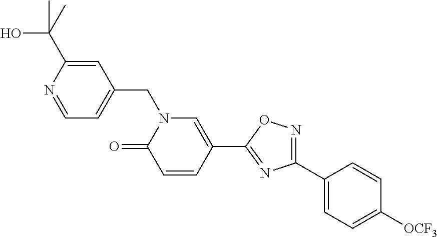 [SMILES: CC(C)(O)c1cc(Cn2cc(-c3nc(-c4ccc(OC(F)(F)F)cc4)no3)ccc2=O)ccn1]

### Steps 1 to 2

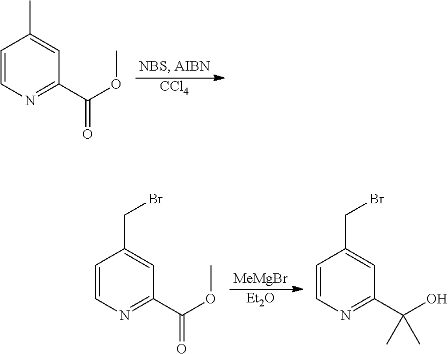

### Step 1: Methyl 4-(bromomethyl)picolinate

To a solution of methyl 4-methylpicolinate (500 mg, 3.31 mmol) in CCl₄ (2 ml) was added NBS (706 mg, 3.97 mmol) followed by AIBN (54.3 mg, 0.331 mmol). The suspension was heated to 80° C. for 4 h. The volatiles were removed under reduced pressure, the residue was treated with Et₂O (10 ml) and the resulting solid was filtered off. The filtrate was concentrated under reduced pressure and the residue was purified first by SiO₂ gel chromatography (10% to 60% EtOAc/Hexanes) and then by prep-HPLC (Mobile phase: A=0.1% TFA/H₂O, B=0.1% TFA/MeCN; Gradient: B=20%-60% in 12 min; Column: C18) to give methyl 4-(bromomethyl)picolinate as a clear oil (122 mg, 16%). MS(ES⁺)

C₈H₈BrNO₂ requires: 229. found: 230 [M+H]⁺.

### Step 2: 2-(4-(Bromomethyl)pyridin-2-yl)propan-2-ol

To a solution of methyl 4-(bromomethyl)picolinate (66 mg, 0.287 mmol) in Et₂O (3 ml) at 0° C. was added dropwise methylmagnesium bromide (3.0 M in Et₂O, 0.287 ml, 0.861 mmol). The reaction was warmed up to RT and stirred for 30 minutes, then quenched with sat. aq. NH₄Cl (5 ml) at 0° C. The mixture was extracted with EtOAc (5 ml), the organic layer was dried with Na₂SO₄, filtered and concentrated under reduced pressure. The residue was purified by SiO₂ gel chromatography (0% to 50% EtOAc/Hexanes) to give 2-(4-(Bromomethyl)pyridin-2-yl)propan-2-ol as a colorless oil (38 mg, 58%). MS (ES⁺) C₉H₁₂BrNO requires: 229. found: 230 [M+H]⁺.

### Step 3

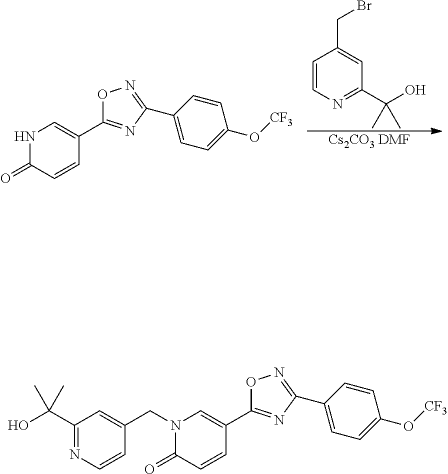

### 1-{[2-(2-Hydroxypropan-2-yl)pyridin-4-yl]methyl}-5-{3-[4-(trifluoromethoxy)phenyl]-1,2,4-oxadiazol-5-yl}-1,2-dihydropyridin-2-one

A mixture of 5-(3-(4-(trifluoromethoxy)phenyl)-1,2,4-oxadiazol-5-yl)pyridin-2(1H)-one (Example 7, Step 1; 50.6 mg, 0.156 mmol), 2-(4-(bromomethyl)pyridin-2-yl)propan-2-ol (36 mg, 0.156 mmol) and Cs₂CO₃ (76 mg, 0.235 mmol) in DMF (200 μl) was stirred at RT for 1 h. The mixture was diluted with water (10 ml) and extracted with EtOAc (3×5 ml). The combined organic layers were washed with brine, dried over Na₂SO₄ and concentrated under reduced pressure. The residue was purified by SiO₂ gel chromatography (15% to 100% MeOH/DCM) to give the title compound as a white solid; MS(ES⁺) C₂₃H₁₉F₃N₄O₄ requires: 472. found: 473 [M+H]⁺; ¹H NMR (600 MHz, Chloroform-d) δ 8.52 (d, J=5.0 Hz, 1H), 8.37 (s, 1H), 8.15 (d, J=8.8 Hz, 2H), 8.08 (d, J=9.6 Hz, 1H), 7.37 (s, 1H), 7.34 (d, J=8.2 Hz, 2H), 7.09 (d, J=5.0 Hz, 1H), 6.79 (d, J=9.5 Hz, 1H), 5.27 (s, 2H), 4.55 (s, 1H), 1.55 (s, 6H).

### EXAMPLE 51

### 1-{[2-(2-Methoxyethoxy)pyridin-4-yl]methyl}-5-{3-[4-(trifluoromethoxy)phenyl]-1,2,4-oxadiazol-5-yl}-1,2-dihydropyridin-2-one

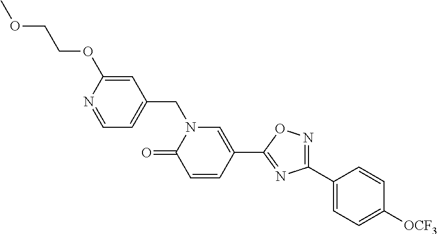 [SMILES: COCCOc1cc(Cn2cc(-c3nc(-c4ccc(OC(F)(F)F)cc4)no3)ccc2=O)ccn1]

### Steps 1 to 2

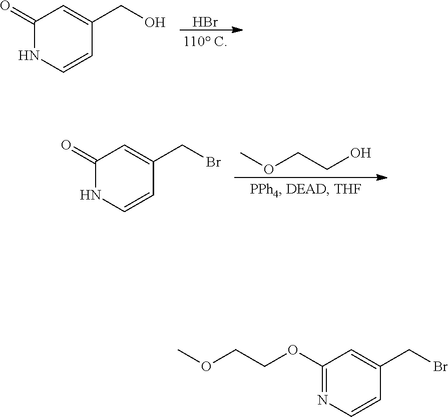

### Step 1: 4-(Bromomethyl)pyridin-2(1H)-one

A mixture of 4-(hydroxymethyl)pyridin-2(1H)-one (500 mg, 4.00 mmol) and HBr (1 ml, 8.84 mmol) was heated to 110° C. for 16 h. The mixture was concentrated under reduced pressure, the residue was treated with water and the resulting suspension was filtered to give 4-(bromomethyl)pyridin-2(1H)-one as a white solid (368 mg, 49%). MS(ES⁺)

C₆H₆BrNO requires: 187. found: 188 [M+H]⁺.

### Step 2: 4-(Bromomethyl)-2-(2-methoxyethoxy)pyridine

To a mixture of 4-(bromomethyl)pyridin-2(1H)-one (50 mg, 0.266 mmol), 2-methoxyethanol (20.24 mg, 0.266 mmol) and triphenylphosphine (105 mg, 0.399 mmol) in THF (3 ml) at 0° C. was added dropwise DEAD (40% wt. in toluene, 0.063 ml, 0.399 mmol). The resulting mixture was stirred at RT for 4 h and the volatiles were removed under reduced pressure. The residue was purified by prep-HPLC (Mobile phase: A=0.1% TFA/H₂O, B=0.1% TFA/MeCN; Gradient: B=30%-70% in 12 min; Column: C18) to give 4-(bromomethyl)-2-(2-methoxyethoxy)pyridine as a clear oil (16 mg, 24%). MS(ES⁺) C₉H₁₂BrNO₂ requires: 245. found: 246 [M+H]⁺.

### Step 3

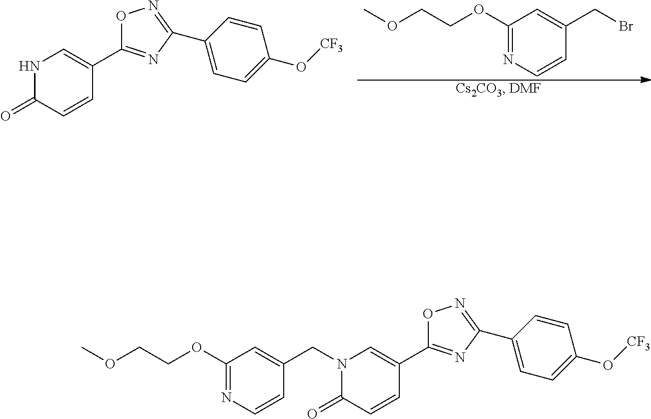

### 1-{[2-(2-Methoxyethoxy)pyridin-4-yl]methyl}-5-{3-[4-(trifluoromethoxy)phenyl]-1,2,4-oxadiazol-5-yl}-1,2-dihydropyridin-2-one

Synthesized in an analogous manner to Example 26; MS(ES⁺) C₂₃H₁₉F₃N₄O₅ requires: 488. found: 489 [M+H]⁺; ¹H NMR (600 MHz, Chloroform-d) δ 8.31 (s, 1H), 8.17 (d, J=8.8 Hz, 2H), 8.06 (d, J=9.5 Hz, 1H), 7.35 (m, 3H), 6.77 (d, J=9.5 Hz, 1H), 6.36 (s, 1H), 6.11 (d, J=7.0 Hz, 1H), 5.06 (s, 2H), 4.09 (t, J=4.8 Hz, 2H), 3.64 (t, J=4.8 Hz, 2H), 3.30 (s, 3H).

### EXAMPLE 52

### 5-(3-(4-(Trifluoromethoxy)phenyl)-1,2,4-oxadiazol-5-yl)-1-(3-vinylbenzyl)pyridin-2(1H)-one

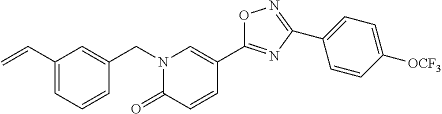 [SMILES: C=Cc1cccc(Cn2cc(-c3nc(-c4ccc(OC(F)(F)F)cc4)no3)ccc2=O)c1]

A mixture of 1-(3-bromobenzyl)-5-(3-(4-(trifluoromethoxy)phenyl)-1,2,4-oxadiazol-5-yl)pyridin-2(1H)-one (Example 31, Step 1; 100 mg, 0.2 mmol), potassium trifluoro-vinyl-boron (27 mg, 0.2 mmol), tetrakis(triphenylphosphine)palladium (46.2 mg, 0.04 mmol) and K₂CO₃ (57 mg, 0.4 mmol) in 1,2-dimethoxyethane (2 mL) and water (1 mL) was stirred at 100° C. under Argon for 2 h. The mixture was then cooled to RT, filtered through a pad of Celite and the filtrate was purified by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to afford the title compound; MS(ES⁺) C₂₃H₁₆F₃N₃O₃ requires: 439. found: 440 [M+H]⁺. ¹H NMR (500 MHz, CDCl₃) δ 8.35 (s, 1H), 8.15 (d, J=8.4 Hz, 2H), 8.02 (d, J=8.4 Hz, 2H), 7.53-7.30 (m, 5H), 6.85-6.61 (m, 2H), 5.77 (d, J=17.5 Hz, 1H), 5.40-5.13 (m, 3H).

### EXAMPLE 53

### 1-(3-(1,2-Dihydroxyethyl)benzyl)-5-(3-(4-(trifluoromethoxy)phenyl)-1,2,4-oxadiazol-5-yl)pyridin-2(1H)-one

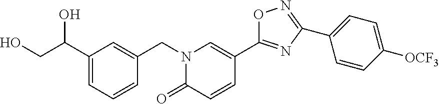 [SMILES: O=c1ccc(-c2nc(-c3ccc(OC(F)(F)F)cc3)no2)cn1Cc1cccc(C(O)CO)c1]

A mixture of 5-(3-(4-(trifluoromethoxy)phenyl)-1,2,4-oxadiazol-5-yl)-1-(3-vinylbenzyl)pyridin-2(1H)-one (Example 10723; 35 mg, 0.08 mmol), 4-Methylmorpholine N-oxide (20 mg, 0.17 mmol) and osmium tetraoxide (0.017 mmol) in tert-butanol (5 mL) was stirred at 30° C. for 16 h. The mixture was then filtered through a pad of Celite, and the filtrate was purified by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to afford the title compound; MS(ES⁺) C₂₃H₁₈F₃N₃O₅ requires: 473. found: 474 [M+H]⁺. ¹H NMR (500 MHz, CDCl₃) δ 8.37 (s, 1H), 8.14 (d, J=8.4 Hz, 2H), 8.02 (d, J=8.5 Hz, 1H), 7.45-7.31 (m, 5H), 7.29 (d, J=7.3 Hz, 1H), 6.74 (d, J=9.4 Hz, 1H), 5.24 (s, 2H), 4.84 (d, J=5.1 Hz, 1H), 3.78 (d, J=8.9 Hz, 1H), 3.64 (m, 1H).

Chiral separation of 1-(3-(1,2-dihydroxyethyl)benzyl)-5-(3-(4-(trifluoromethoxy)phenyl)-1,2,4-oxadiazol-5-yl)pyridin-2(1H)-one by chiral prep-HPLC (Mobile phase: A=0.1% DEA/Hexanes, B=0.1% DEA/EtOH; A:B=82:18; 20 ml/min; Column: AY-H-2 (20×250 mm, Daicel) gave the following enantiomerically pure products: (1R)- or (1S)-1-(3-(1,2-dihydroxyethyl)benzyl)-5-(3-(4-(trifluoromethoxy)phenyl)-1,2,4-oxadiazol-5-yl)pyridin-2(1H)-one (Example 53a): Rt=26 minutes; (1S)- or (1R)-1-(3-(1,2-dihydroxyethyl)benzyl)-5-(3-(4-(trifluoromethoxy)phenyl)-1,2,4-oxadiazol-5-yl)pyridin-2(1H)-one (Example 53b): Rt=35 minutes.

### EXAMPLE 54

### 1-(3-(Allyloxy)benzyl)-5-(3-(4-(trifluoromethoxy)phenyl)-1,2,4-oxadiazol-5-yl)pyridin-2(1H)-one

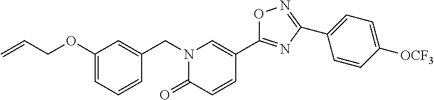 [SMILES: C=CCOc1cccc(Cn2cc(-c3nc(-c4ccc(OC(F)(F)F)cc4)no3)ccc2=O)c1]

To a solution of 3-((5-methyl-3-(3-(4-(trifluoromethoxy)phenyl)-1,2,4-oxadiazol-5-yl)-1H-1,2,4-triazol-1-yl)methyl)phenol (Example 32; 100 mg, 0.24 mmol) and 3-bromoprop-1-ene (57.6 mg, 0.48 mmol) in DCM (5 mL) and H₂O (5 ml), were added tetrabutylammonium hydrogen sulfate (816 mg, 2.4 mmol) and K₂CO₃ (66 mg, 0.48 mmol). The mixture was stirred at RT for 16 h, then it was diluted with H₂O (20 mL) and extracted with DCM (3×20 mL). The combined organic layers were washed with water (20 mL) and brine (20 mL), dried over Na₂SO₄, filtered, and concentrated under reduced pressure. The residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid; MS(ES⁺) C₂₄H₁₈F₃N₃O₄ requires: 469. found: 470 [M+H]⁺. ¹H NMR (500 MHz, CDCl₃) δ 8.33 (d, J=2.4 Hz, 1H), 8.15 (d, J=8.7 Hz, 2H), 8.02 (dd, J=9.5, 2.5 Hz, 1H), 7.37-7.27 (m, 3H), 6.99-6.87 (m, 3H), 6.76 (d, J=9.6 Hz, 1H), 6.03 (m, 1H), 5.41 (dd, J=17.3, 1.3 Hz, 1H), 5.29 (d, J=10.5 Hz, 1H), 5.21 (s, 2H), 4.53 (d, J=5.3 Hz, 2H).

### EXAMPLE 55

### 1-(3-(2,3-Dihydroxypropoxy)benzyl)-5-(3-(4-(trifluoromethoxy)phenyl)-1,2,4-oxadiazol-5-yl)pyridin-2(1H)-one

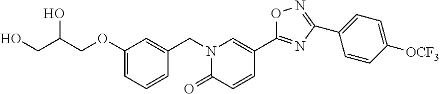 [SMILES: O=c1ccc(-c2nc(-c3ccc(OC(F)(F)F)cc3)no2)cn1Cc1cccc(OCC(O)CO)c1]

A mixture of 1-(3-(allyloxy)benzyl)-5-(3-(4-(trifluoromethoxy)phenyl)-1,2,4-oxadiazol-5-yl)pyridin-2(1H)-one (Example 54; 40 mg, 0.09 mmol) osmium(VIII) oxide (2.6 mg, 0.01 mmol) and 4-Methylmorpholine N-oxide (21 mg, 0.18 mmol) in acetone (3 mL) and H₂O (3 ml) was stirred at RT for 3 h. The mixture was then diluted with H₂O (20 mL) and extracted with DCM (3×20 mL). The combined organic layers were washed with water (20 mL) and brine (20 mL), dried over Na₂SO₄, filtered, and concentrated under reduced pressure. The residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid; MS(ES⁺) C₂₄H₂₀F₃N₃O₆ requires: 503. found: 504 [M+H]⁺. ¹H NMR (500 MHz, CDCl₃) δ 8.34 (s, 1H), 8.15 (d, J=8.7 Hz, 2H), 8.03 (dd, J=9.5, 2.4 Hz, 1H), 7.32 (m, 3H), 7.00-6.87 (m, 3H), 6.76 (d, J=9.6 Hz, 1H), 5.21 (s, 2H), 4.16-4.00 (m, 3H), 3.84 (m, 1H), 3.75 (dd, J=11.4, 5.4 Hz, 1H).

Chiral separation of 1-(3-(2,3-dihydroxypropoxy)benzyl)-5-(3-(4-(trifluoromethoxy)phenyl)-1,2,4-oxadiazol-5-yl)pyridin-2(1H)-one by preparative SFC (Mobile phase: 1/1 CO₂/MeOH (0.1% DEA); 130 g/min; Column: Regiscell (5 μM; 50×250 mm) gave the following enantiomerically pure products: (2R)- or (2S)-1-(3-(2,3-dihydroxypropoxy)benzyl)-5-(3-(4-(trifluoromethoxy)phenyl)-1,2,4-oxadiazol-5-yl)pyridin-2(1H)-one (Example 55a): Rt=8.8 minutes; (2S)- or (2R)-1-(3-(2,3-dihydroxypropoxy)benzyl)-5-(3-(4-(trifluoromethoxy)phenyl)-1,2,4-oxadiazol-5-yl)pyridin-2(1H)-one (Example 55b): Rt=11.9 minutes.

### EXAMPLE 56

### 5-(3-(4-(Trifluoromethoxy)phenyl)-1,2,4-oxadiazol-5-yl)-1-(3-vinylbenzyl)pyridin-2(1H)-one

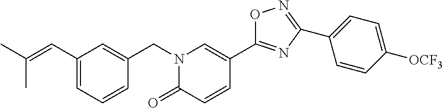 [SMILES: CC(C)=Cc1cccc(Cn2cc(-c3nc(-c4ccc(OC(F)(F)F)cc4)no3)ccc2=O)c1]

A mixture of 1-(3-bromobenzyl)-5-(3-(4-(trifluoromethoxy)phenyl)-1,2,4-oxadiazol-5-yl)pyridin-2(1H)-one (Example 31, Step 1; 200 mg, 0.403 mmol), 4,4,5,5-tetramethyl-2-(2-methylprop-1-enyl)-1,3,2-dioxaborolane (81 mg, 0.403 mmol), tetrakis(triphenylphosphine)palladium (194 mg, 0.08 mmol) and K₂CO₃ (114 mg, 0.406 mmol) in 1,2-dimethoxyethane (1.5 mL) and water (0.5 mL) was stirred at 100° C. under Argon for 3 h. The mixture was then cooled to RT, filtered through a pad of Celite, and the filtrate was purified by prep-HPLC (Mobile phase: A=0.1% ammonium hydroxide/H₂O, B=MeCN; Gradient: B=60% to 95% in 18 min; Column: XBridge (C18, 5 um, 30 mm×150 mm) to afford the title compound; MS(ES⁺) C₂₅H₂₀F₃N₃O₃ requires: 467.15. found: 468.1 [M+H]⁺. ¹H NMR (500 MHz, CDCl₃) δ 8.35 (d, J=2.4 Hz, 1H), 8.15 (d, J=8.7 Hz, 2H), 8.02 (dd, J=9.5, 2.5 Hz, 1H), 7.34 (m, 3H), 7.20 (m, 3H), 6.75 (d, J=9.5 Hz, 1H), 6.25 (bs, 1H), 5.23 (s, 2H), 1.87 (d, J=28.1 Hz, 6H).

### EXAMPLE 57

### 1-(3-(1,2-Dihydroxy-2-methylpropyl)benzyl)-5-(3-(4-(trifluoromethoxy)phenyl)-1,2,4-oxadiazol-5-yl)pyridin-2(1H)-one

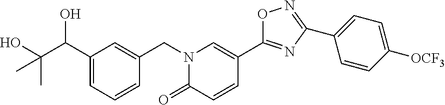 [SMILES: CC(C)(O)C(O)c1cccc(Cn2cc(-c3nc(-c4ccc(OC(F)(F)F)cc4)no3)ccc2=O)c1]

A mixture of 1-(3-(2-methylprop-1-enyl)benzyl)-5-(3-(4-(trifluoromethoxy)phenyl)-1,2,4-oxadiazol-5-yl)pyridin-2(1H)-one (Example 56; 30 mg, 0.064 mmol) osmium tetraoxide (4.4 mg, 0.017 mmol) and 4-methylmorpholine N-oxide (20 mg, 0.17 mmol) in tert-butanol (3 mL) was stirred at 30° C. for 16 h. The mixture was then filtered through a pad of Celite, and the filtrate was purified by prep-HPLC (Mobile phase: A=0.1% ammonium hydroxide/H₂O, B=MeCN; Gradient: B=60% to 95% in 18 min; Column: XBridge (C18, 5 um, 30 mm×150 mm) to afford the title compound; MS(ES⁺) C₂₅H₂₂F₃N₃O₅ requires: 501.15. found: 502.1 [M+H]⁺. ¹H NMR (500 MHz, CDCl₃) δ 8.35 (d, J=2.2 Hz, 1H), 8.14 (d, J=8.6 Hz, 2H), 8.01 (dd, J=9.5, 2.3 Hz, 1H), 7.43 (s, 1H), 7.35 (m, 4H), 7.28 (m, 1H), 6.74 (d, J=9.6 Hz, 1H), 5.24 (s, 2H), 4.54 (s, 1H), 1.24 (s, 3H), 1.08 (s, 3H).

### EXAMPLE 58

### 1-(3-(4-Hydroxypiperidin-1-yl)benzyl)-5-(3-(4-(trifluoromethoxy)phenyl)-1,2,4-oxadiazol-5-yl)pyridin-2(1H)-one

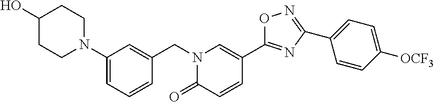 [SMILES: O=c1ccc(-c2nc(-c3ccc(OC(F)(F)F)cc3)no2)cn1Cc1cccc(N2CCC(O)CC2)c1]

To a solution of 1-(3-bromobenzyl)-5-(3-(4-(trifluoromethoxy)phenyl)-1,2,4-oxadiazol-5-yl)pyridin-2(1H)-one (Example 31, Step 1; 100 mg, 0.21 mmol) in toluene (3 mL), were added 2-dicyclohexylphosphino-2′,6′-diisopropoxy-1,1′-biphenyl (10 mg, 0.021 mmol), tris(dibenzylideneacetone)dipalladium (19 mg, 0.021 mmol), piperidin-4-ol (100 mg, 1.0 mmol), and sodium tert-butoxide (40 mg, 0.42 mmol). The mixture was stirred at 100° C. for 3 h, then concentrated under reduced pressure, diluted with H₂O (20 mL) and extracted with EtOAc (3×20 mL). The combined organic layers were washed with water (20 mL) and brine (20 mL), dried over Na₂SO₄, filtered, and concentrated under reduced pressure. The residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid; MS(ES⁺) C₂₆H₂₃F₃N₄O₄ requires: 512. found: 513 [M+H]⁺; ¹H NMR (500 MHz, CDCl₃) δ 8.33 (d, J=2.3 Hz, 1H), 8.14 (d, J=8.8 Hz, 2H), 8.01 (dd, J=9.6, 2.5 Hz, 1H), 7.34 (d, J=8.3 Hz, 2H), 7.26 (m, 1H), 6.96 (s, 1H), 6.92 (d, J=8.1 Hz, 1H), 6.80 (d, J=7.4 Hz, 1H), 6.74 (d, J=9.6 Hz, 1H), 5.18 (s, 2H), 3.86 (s, 1H), 3.59-3.55 (m, 2H), 2.98-2.92 (m, 2H), 2.02-1.99 (m, 2H), 1.71-1.65 (m, 2H).

### EXAMPLE 59

### 1-(3-(piperazin-1-yl)benzyl)-5-(3-(4-(trifluoromethoxy)phenyl)-1,2,4-oxadiazol-5-yl)pyridin-2(1H)-one

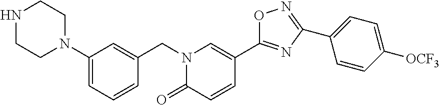 [SMILES: O=c1ccc(-c2nc(-c3ccc(OC(F)(F)F)cc3)no2)cn1Cc1cccc(N2CCNCC2)c1]

A suspension of 1-(3-bromobenzyl)-5-(3-(4-(trifluoromethoxy)phenyl)-1,2,4-oxadiazol-5-yl)pyridin-2(1H)-one (Example 31, Step 1; 200 mg, 0.4 mmol), piperazine (180 mg, 2.09 mmol), tris(dibenzylideneacetone)dipalladium (80 mg, 0.08 mmol), tri-tert-butylphosphine tetrafluoroborate (110 mg, 0.38 mmol) and sodium tert-butoxide (80 mg, 0.833 mmol) in toluene (5 mL) was degassed and refilled with argon three times, and stirred at 140° C. for 4 h. The mixture was then cooled to RT, filtered through a pad of Celite. The filtrate was purified by prep-HPLC (Mobile phase: A=0.1% ammonium hydroxide/H2O, B=MeCN; Gradient: B=60% to 95% in 18 min; Column: XBridge (C18, Sum, 30 mm×150 mm) to afford the title compound; MS(ES⁺) C₂₅H₂₂F₃N₅O₃ requires: 497. found: 498 [M+H]⁺. ¹H NMR (500 MHz, MeOD) δ 8.82 (d, J=2.4 Hz, 1H), 8.35-8.15 (m, 3H), 7.49 (d, J=8.4 Hz, 2H), 7.34 (m, 1H), 7.13 (s, 1H), 7.07-6.96 (m, 2H), 6.76 (d, J=9.5 Hz, 1H), 5.30 (s, 2H), 3.46-3.41 (m, 4H), 3.40-3.37 (m, 4H).

### EXAMPLE 60

### 1-(3-(piperazin-1-yl)benzyl)-5-(3-(4-(trifluoromethoxy)phenyl)-1,2,4-oxadiazol-5-yl)pyridin-2(1H)-one

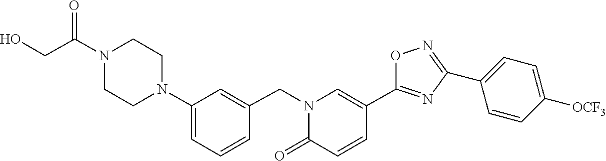 [SMILES: O=C(CO)N1CCN(c2cccc(Cn3cc(-c4nc(-c5ccc(OC(F)(F)F)cc5)no4)ccc3=O)c2)CC1]

A mixture of 1-(3-(piperazin-1-yl)benzyl)-5-(3-(4-(trifluoromethoxy)phenyl)-1,2,4-oxadiazol-5-yl)pyridin-2(1H)-one (Example 59; 20 mg, 0.04 mmol), 2-hydroxyacetic acid (16 mg, 0.06 mmol), 2-(7-aza-1H-benzotriazole-1-yl)-1,1,3,3-tetramethyluronium hexafluorophosphate in (16 mg, 0.04 mmol) and triethylamine (13 mg, 0.12 mmol) in DMF (3 mL) was stirred at RT for 16 h. The mixture was purified directly by prep-HPLC (Mobile phase: A=0.1% ammonium hydroxide/H2O, B=MeCN; Gradient: B=60% to 95% in 18 min; Column: XBridge (C18, Sum, 30 mm×150 mm) to give the title compound; MS(ES⁺) C₂₇H₂₄F₃N₅O₅ requires: 555. found: 556 [M+H]⁺. ¹H NMR (500 MHz, CDCl₃) δ 8.34 (bs, 1H), 8.15 (d, J=8.4 Hz, 2H), 8.03 (bd, J=8.0 Hz, 1H), 7.41-7.27 (m, 3H), 6.97 (s, 1H), 6.90 (d, J=8.6 Hz, 2H), 6.75 (d, J=9.4 Hz, 1H), 5.20 (s, 2H), 4.21 (s, 2H), 3.83 (m, 2H), 3.61 (m, 1H), 3.43 (m, 2H), 3.21 (m, 4H).

### EXAMPLE 61

### 1-(3-(4-Methylpiperazin-1-yl)benzyl)-5-(3-(4-(trifluoromethoxy)phenyl)-1,2,4-oxadiazol-5-yl)pyridin-2(1H)-one

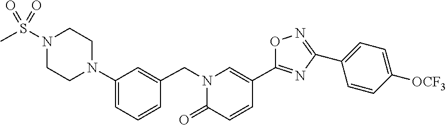 [SMILES: CS(=O)(=O)N1CCN(c2cccc(Cn3cc(-c4nc(-c5ccc(OC(F)(F)F)cc5)no4)ccc3=O)c2)CC1]

To a solution of 1-(3-(piperazin-1-yl)benzyl)-5-(3-(4-(trifluoromethoxy)phenyl)-1,2,4-oxadiazol-5-yl)pyridin-2(1H)-one (Example 59; 100 mg, 0.2 mmol) and triethylamine (120 mg, 1.0 mmol) in DCM (5 mL) at 0° C. was added methanesulfonyl chloride (28 mg, 0.24 mmol). The resulting mixture was stirred at RT for 16 h and then concentrated under reduced pressure. The residue was purified by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to give the title compound; MS(ES⁺) C₂₆H₂₄F₃N₅O₅S requires: 575. found: 576 [M+H]⁺. ¹H NMR (500 MHz, DMSO) δ 8.98 (d, J=2.5 Hz, 1H), 8.18 (d, J=8.8 Hz, 2H), 8.06 (dd, J=9.6, 2.5 Hz, 1H), 7.60 (d, J=8.4 Hz, 2H), 7.22 (m, 1H), 7.08 (s, 1H), 6.92 (d, J=8.0 Hz, 1H), 6.79 (d, J=7.6 Hz, 1H), 6.66 (d, J=9.6 Hz, 1H), 5.22 (s, 2H), 3.24 (s, 8H), 2.92 (s, 3H).

### EXAMPLE 62

### 1-(3-(3-Amino-2-hydroxypropoxy)benzyl)-5-(3-(4-(trifluoromethoxy)phenyl)-1,2,4-oxadiazol-5-yl)pyridin-2(1H)-one

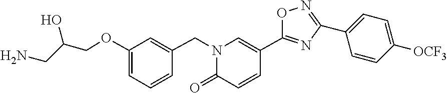 [SMILES: NCC(O)COc1cccc(Cn2cc(-c3nc(-c4ccc(OC(F)(F)F)cc4)no3)ccc2=O)c1]

### Step 1

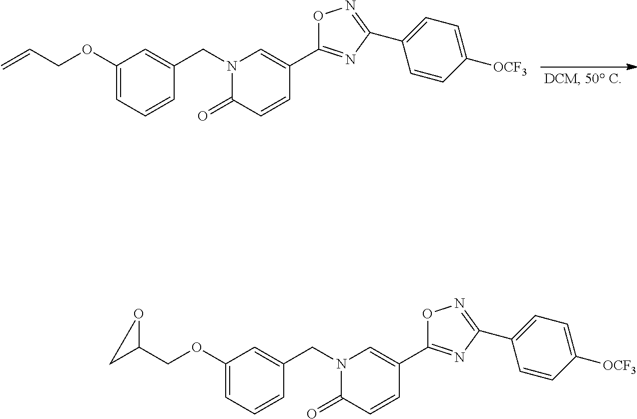 [SMILES: C=CCOc1cccc(Cn2cc(-c3nc(-c4ccc(OC(F)(F)F)cc4)no3)ccc2=O)c1 ; O=c1ccc(-c2nc(-c3ccc(OC(F)(F)F)cc3)no2)cn1Cc1cccc(OCC2CO2)c1]

### 1-(3-(Oxiran-2-ylmethoxy)benzyl)-5-(3-(4-(trifluoromethoxy)phenyl)-1,2,4-oxadiazol-5-yl)pyridin-2(1H)-one

To a solution of 1-(3-(allyloxy)benzyl)-5-(3-(4-(trifluoromethoxy)phenyl)-1,2,4-oxadiazol-5-yl)pyridin-2(1H)-one (Example 54; 100 mg, 0.21 mmol) in DCM (5 mL) was added 3-chlorobenzoperoxoic acid (840 mg, 4.88 mmol). The mixture was stirred at 50° C. for 12 h and then concentrated under reduced pressure. The residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid MS(ES⁺) C₂₄H₁₈F₃N₃O₅ requires: 485. found: 486 [M+H]⁺.

### Step 2

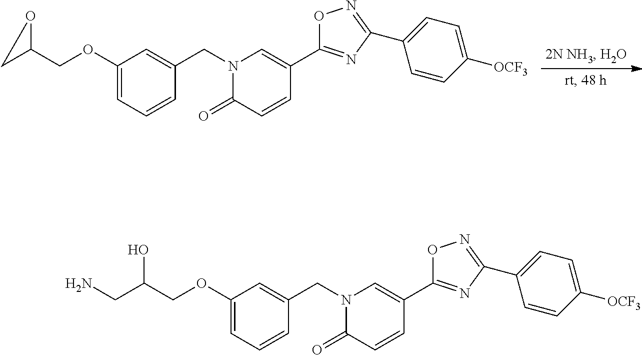 [SMILES: O=c1ccc(-c2nc(-c3ccc(OC(F)(F)F)cc3)no2)cn1Cc1cccc(OCC2CO2)c1 ; NCC(O)COc1cccc(Cn2cc(-c3nc(-c4ccc(OC(F)(F)F)cc4)no3)ccc2=O)c1]

### 1-(3-(3-Amino-2-hydroxypropoxy)benzyl)-5-(3-(4-(trifluoromethoxy)phenyl)-1,2,4-oxadiazol-5-yl)pyridin-2(1H)-one

A solution of 1-(3-(oxiran-2-ylmethoxy)benzyl)-5-(3-(4-(trifluoromethoxy)phenyl)-1,2,4-oxadiazol-5-yl)pyridin-2(1H)-one (50 mg, 0.1 mmol) in 2N aq. ammonia (5 mL) was stirred at RT for 48 h. The volatiles were removed under reduced pressure and the residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid; MS(ES⁺) C₂₄H₂₁F₃N₄O₅ requires: 502. found: 503 [M+H]⁺. ¹H NMR (500 MHz, MeOD) δ 8.71 (d, J=2.3 Hz, 1H), 8.40, (bs, 2H), 8.17-8.04 (m, 3H), 7.37 (d, J=8.2 Hz, 2H), 7.22 (m, 1H), 6.93 (m, 2H), 6.85 (d, J=8.8 Hz, 1H), 6.64 (d, J=9.5 Hz, 1H), 5.20 (s, 2H), 4.04 (m, 1H), 3.98-3.83 (m, 2H), 3.09 (bd, J=15.7 Hz, 1H), 2.88 (m, 1H).

### EXAMPLE 63

### 1-(3-(4-Methylpiperazin-1-yl)benzyl)-5-(3-(4-(trifluoromethyl)phenyl)-1,2,4-oxadiazol-5-yl)pyridin-2(1H)-one

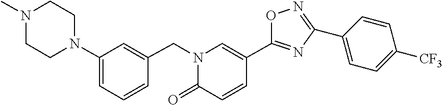 [SMILES: CN1CCN(c2cccc(Cn3cc(-c4nc(-c5ccc(C(F)(F)F)cc5)no4)ccc3=O)c2)CC1]

### Step 1

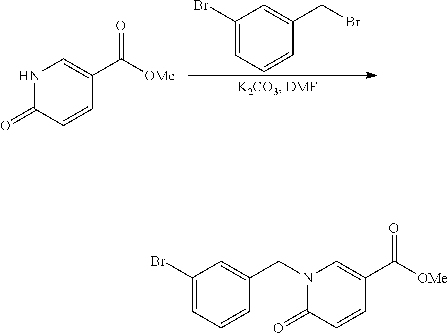 [SMILES: COC(=O)c1ccc(=O)[nH]c1 ; BrCc1cccc(Br)c1 ; COC(=O)c1ccc(=O)n(Cc2cccc(Br)c2)c1]

### Methyl 1-(3-bromobenzyl)-6-oxo-1,6-dihydropyridine-3-carboxylate

To a suspension of K₂CO₃ (1.35 g, 9.80 mmol) and methyl 6-oxo-1,6-dihydropyridine-3-carboxylate (1 g, 6.5 mmol) in DMF (1.89 mL) was added 1-bromo-3-(bromomethyl)benzene (1.96 g, 7.84 mmol). The mixture was stirred at RT for 12 h, filtered through a pad of Celite and concentrated under reduced pressure. The residue was purified by SiO₂ gel chromatography (0% to 40% EtOAc/Hexanes) to give the title compound as a pale yellow oil (1.95 g, 93%): MS(ES⁺) C₁₄H₁₂BrNO₃ requires: 321. found: 322/324 [M+H]⁺.

### Step 2

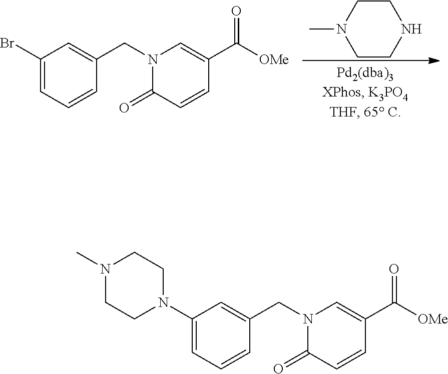

### Methyl 1-(3-(4-methylpiperazin-1-yl)benzyl)-6-oxo-1,6-dihydropyridine-3-carboxylate

To a solution of methyl 1-(3-bromobenzyl)-6-oxo-1,6-dihydropyridine-3-carboxylate (1 g, 3.10 mmol) in THF (15.5 mL) previously degassed with N₂, were added K₃PO₄ (1.45 g, 6.83 mmol), Pd₂(dba)₃ (0.28 g, 0.310 mmol), XPhos (0.30 g, 0.621 mmol) and 1-methylpiperazine (0.516 ml, 4.66 mmol). The mixture was heated at 65° C. for 72 h, cooled to RT, filtered through a pad of Celite and concentrated under reduced pressure. The residue was purified by SiO₂ gel chromatography (0% to 20% MeOH/DCM) to give the title compound as an orange solid (1.04 g, 98%): MS(ES⁺) C₁₉H₂₃N₃O₃ requires: 341. found: 342 [M+H]⁺.

### Step 3

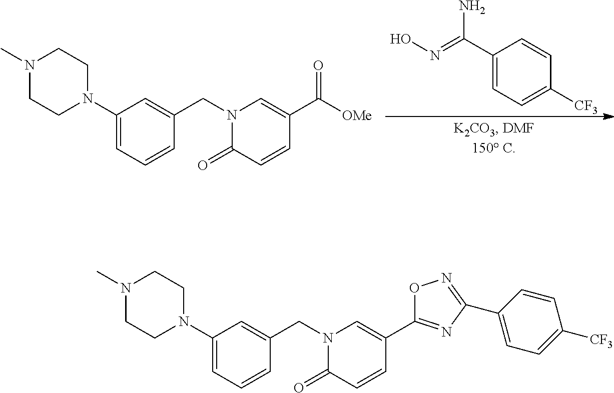 [SMILES: COC(=O)c1ccc(=O)n(Cc2cccc(N3CCN(C)CC3)c2)c1 ; N/C(=N\O)c1ccc(C(F)(F)F)cc1 ; CN1CCN(c2cccc(Cn3cc(-c4nc(-c5ccc(C(F)(F)F)cc5)no4)ccc3=O)c2)CC1]

### 1-(3-(4-Methylpiperazin-1-yl)benzyl)-5-(3-(4-(trifluoromethyl)phenyl)-1,2,4-oxadiazol-5-yl)pyridin-2(1H)-one

To a suspension of K₂CO₃ (16.2 mg, 0.117 mmol) and methyl 1-(3-(4-methylpiperazin-1-yl)benzyl)-6-oxo-1,6-dihydropyridine-3-carboxylate (20 mg, 0.059 mmol) in DMF (0.293 mL) was added (Z)—N′-hydroxy-4-(trifluoromethyl)benzimidamide (14.3 mg, 0.070 mmol). The mixture was heated to 150° C. for 12 h, cooled to RT and partitioned between EtOAc and H₂O. The mixture was extracted with EtOAc (3×1 mL), the combined organic layers were washed with H₂O (3×1 mL), brine (1 mL), dried (Na₂SO₄) and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to provide the title compound; ¹H NMR (600 MHz, Chloroform-d) δ 8.37 (d, J=2.5 Hz, 1H), 8.23 (d, J=7.7 Hz, 2H), 8.04 (dd, J=9.6, 2.5 Hz, 1H), 7.77 (d, J=8.1 Hz, 2H), 7.32 (m, 1H), 6.99 (m, 1H), 6.95 (m, 1H), 6.90 (m, 1H), 6.76 (d, J=9.6 Hz, 1H), 5.20 (s, 2H), 3.67 (bd, J=12.2 Hz, 4H), 3.39-3.28 (m, 2H), 3.00 (bd, J=12.3 Hz, 2H), 2.87 (s, 3H); MS(ES⁺) C₂₆H₂₄F₃N₅O₂ requires: 495. found: 496 [M+H]⁺.

### EXAMPLE 64

### 1-(3-(4-Methylpiperazin-1-yl)benzyl)-5-(5-(4-(trifluoromethyl)phenyl)-1,2,4-oxadiazol-3-yl)pyridin-2(1H)-one

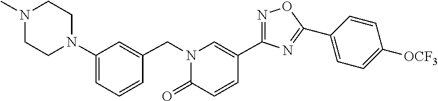 [SMILES: CN1CCN(c2cccc(Cn3cc(-c4noc(-c5ccc(OC(F)(F)F)cc5)n4)ccc3=O)c2)CC1]

### Step 1

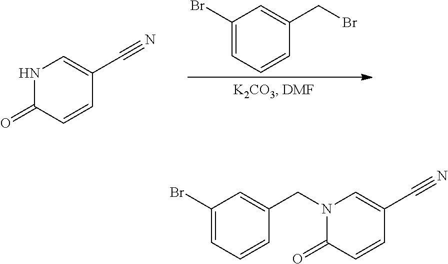 [SMILES: N#Cc1ccc(=O)[nH]c1 ; BrCc1cccc(Br)c1 ; N#Cc1ccc(=O)n(Cc2cccc(Br)c2)c1]

### 1-(3-Bromobenzyl)-6-oxo-1,6-dihydropyridine-3-carbonitrile

To a mixture of K₂CO₃ (345 mg, 2.50 mmol) and 6-oxo-1,6-dihydropyridine-3-carbonitrile (200 mg, 1.67 mmol) in DMF (8.25 mL) was added 1-bromo-3-(bromomethyl)benzene (500 mg, 2.00 mmol). The mixture was stirred at RT for 12 h, filtered through a pad of Celite and concentrated under reduced pressure. The residue was purified by SiO₂ gel chromatography (0% to 40% EtOAc/Hexanes) to give the title compound as a white solid (350 mg, 73%): MS(ES⁺) C₁₃H₉BrN₂O requires: 288. found: 289/291 [M+H]⁺.

### Step 2

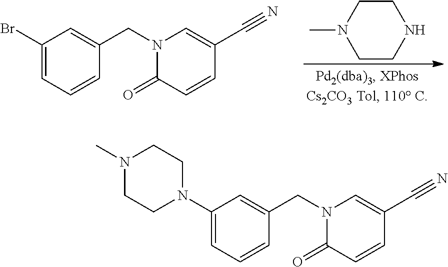

### 1-(3-(4-Methylpiperazin-1-yl)benzyl)-6-oxo-1,6-dihydropyridine-3-carbonitrile

To a mixture of Cs₂CO₃ (248 mg, 0.761 mmol), 1-(3-bromobenzyl)-6-oxo-1,6-dihydropyridine-3-carbonitrile (100 mg, 0.346 mmol), Pd₂(dba)₃ (32 mg, 0.035 mmol), and XPhos (33 mg, 0.069 mmol) in toluene (1.7 mL) previously degassed with N₂ was added 1-methylpiperazine (58 μl, 0.52 mmol). The reaction mixture was heated to 110° C. for 3 h, cooled to RT, filtered through silica gel (20% MeOH/CH₂Cl₂) and concentrated under reduced pressure. The residue was purified by SiO₂ gel chromatography (0% to 20% MeOH/DCM) to give the title compound (85 mg, 80%); MS(ES⁺) C₁₈H₂₀N₄O requires: 308. found: 309 [M+H]⁺.

### Step 3

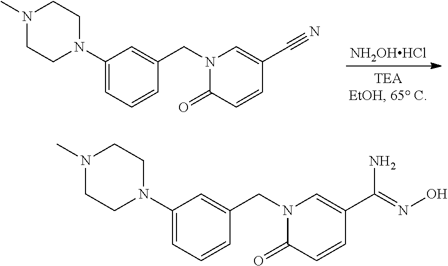

### (Z)—N′-Hydroxy-1-(3-(4-methylpiperazin-1-yl)benzyl)-6-oxo-1,6-dihydropyridine-3-carboximidamide

To a solution of 1-(3-(4-methylpiperazin-1-yl)benzyl)-6-oxo-1,6-dihydropyridine-3-carbonitrile (100 mg, 0.324 mmol) and hydroxylamine hydrochloride (27 mg, 0.39 mmol) in EtOH (1.6 mL) was added Et₃N (54 μL, 0.39 mmol). The mixture as heated to 65° C. for 30 minutes, then stirred at RT for 12 h, diluted with H₂O (3 mL) and extracted with EtOAc (3×3 mL). The organic layer was washed with H₂O (2 mL) and brine (2 mL) and concentrated to give the title compound, which was taken to the next step without further purification; MS(ES⁺) C₁₈H₂₃N₅O₂ requires: 341. found: 342 [M+H]⁺.

### Step 4

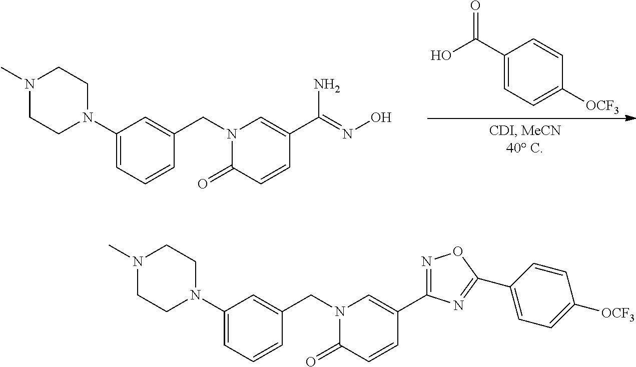 [SMILES: CN1CCN(c2cccc(Cn3cc(/C(N)=N/O)ccc3=O)c2)CC1 ; O=C(O)c1ccc(OC(F)(F)F)cc1 ; CN1CCN(c2cccc(Cn3cc(-c4noc(-c5ccc(OC(F)(F)F)cc5)n4)ccc3=O)c2)CC1]

### 1-(3-(4-Methylpiperazin-1-yl)benzyl)-5-(5-(4-(trifluoromethyl)phenyl)-1,2,4-oxadiazol-3-yl)pyridin-2(1H)-one

To a solution of 4-(trifluoromethoxy)benzoic acid (25 mg, 0.12 mmol) in MeCN (1.5 mL) was added CDI (24 mg, 0.15 mmol), and the mixture was heated to 40° C. for 1 h. (Z)—N′-hydroxy-1-(3-(4-methylpiperazin-1-yl)benzyl)-6-oxo-1,6-dihydropyridine-3-carboximidamide (50 mg, 0.15 mmol) was then added and the mixture was heated at 40° C. for 1 h. DMF (1.5 mL) was added and the mixture was heated to 150° C. for further 2 h, then cooled to RT, diluted with H₂O (2 mL) and extracted with EtOAc (3×2 mL). The organic layer was washed with H₂O (1 mL) and brine (1 mL), dried (Na₂SO₄) and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to give the title compound; MS(ES⁺) C₂₆H₂₄F₃N₅O₂ requires: 495. found: 496 [M+H]⁺; ¹H NMR (600 MHz, Chloroform-d) δ 8.28 (d, J=2.4 Hz, 1H), 8.26-8.18 (m, 3H), 8.16-8.10 (m, 1H), 8.04 (dd, J=9.5, 2.5 Hz, 1H), 7.39 (d, J=8.1 Hz, 2H), 6.99 (m, 1H), 6.95 (d, J=7.8 Hz, 1H), 6.87 (dd, J=8.0, 2.1 Hz, 1H), 6.75 (d, J=9.5 Hz, 1H), 5.20 (s, 2H), 3.66 (bd, J=12.5 Hz, 2H), 3.42-3.28 (m, 4H), 3.04-2.92 (m, 2H), 2.87 (s, 3H).

### EXAMPLE 65

### 1-(3-(2-Methoxyethoxy)benzyl)-5-(5-(4-(trifluoromethyl)phenyl)-1,2,4-oxadiazol-3-yl)pyridin-2(1H)-one

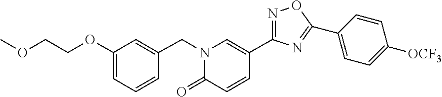 [SMILES: COCCOc1cccc(Cn2cc(-c3noc(-c4ccc(OC(F)(F)F)cc4)n3)ccc2=O)c1]

### Steps 1 to 3

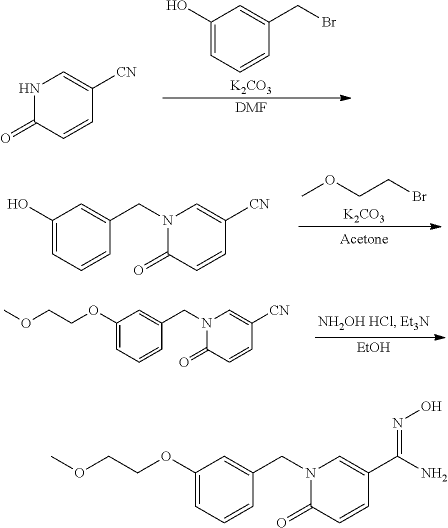

### Step 1: 1-(3-Hydroxybenzyl)-6-oxo-1,6-dihydropyridine-3-carbonitrile

A mixture of 6-oxo-1,6-dihydropyridine-3-carbonitrile (3.800 g, 31.6 mmol) and K₂CO₃ (8.75 g, 63.3 mmol) in DMF (100 ml) was stirred at RT for 5 minutes. 3-(Bromomethyl)phenol (6.51 g, 34.8 mmol) was then added and the reaction was heated at 65° C. for 4 h, diluted with EtOAc (500 mL) and washed with water (4×400 mL). The organic layer was dried over Na₂SO₄, filtered, and concentrated under reduced pressure. The residue was triturated with ether to afford the title compound (4.29 g, 18.96 mmol, 59.9% yield), which was used without further purification. MS(ES⁺) C₁₃H₁₀N₂O₂ requires: 226. found 227 [M+H]⁺.

### Step 2: 1-(3-(2-Methoxyethoxy)benzyl)-6-oxo-1,6-dihydropyridine-3-carbonitrile

A mixture of 1-(3-hydroxybenzyl)-6-oxo-1,6-dihydropyridine-3-carbonitrile (1.00 g, 4.42 mmol) and K₂CO₃ (1.222 g, 8.84 mmol) in acetone (40 ml) was stirred at RT for 5 minutes. 1-bromo-2-methoxyethane (1.246 ml, 13.26 mmol) was added, the reaction mixture was heated at 65° C. for 12 h and then filtered through a pad of Celite and concentrated under reduced pressure. The residue was purified by SiO₂ gel chromatography (EtOAc/Hexanes 0%-60% EtOAc/hex) to afford the title compound (1.257 g, 4.42 mmol, 100% yield) as a colorless oil. MS(ES⁺) C₁₆H₁₆N₂O₃ requires: 284. found 285 [M+H]⁺.

### Step 3: N′-Hydroxy-1-(3-(2-methoxyethoxy)benzyl)-6-oxo-1,6-dihydropyridine-3-carboximidamide

To a solution of 1-(3-(2-methoxyethoxy)benzyl)-6-oxo-1,6-dihydropyridine-3-carbonitrile (1.257 g, 4.42 mmol) and hydroxylamine hydrochloride (0.338 g, 4.86 mmol) in EtOH (11 ml) was added triethylamine (0.678 ml, 4.86 mmol). The mixture was heated at 65° C. for 2 hours, cooled to RT, concentrated to a volume of 2 mL and then partitioned between EtOAc (30 mL) and water (30 mL). The aqueous layer was extracted with EtOAc (3×30 mL). The combined organic layers were washed with water (4×30 mL), brine (30 mL), dried over Na₂SO₄ and concentrated under reduced pressure. The residue was dissolved in a minimal amount of DCM and Hexanes resulting in the formation of a white solid precipitate. The mixture was concentrated under reduced pressure to afford the title compound (645 mg, 2.033 mmol, 46.0% yield) as a white solid. MS (ES⁺) C₁₆H₁₉N₃O₄ requires: 317. found 318 [M+H]⁺.

### Step 4

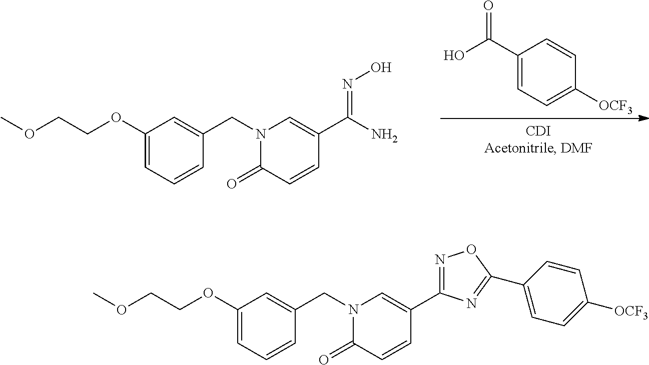 [SMILES: COCCOc1cccc(Cn2cc(/C(N)=N/O)ccc2=O)c1 ; O=C(O)c1ccc(OC(F)(F)F)cc1 ; COCCOc1cccc(Cn2cc(-c3noc(-c4ccc(OC(F)(F)F)cc4)n3)ccc2=O)c1]

### 1-(3-(2-Methoxyethoxy)benzyl)-5-(5-(4-(trifluoromethoxy)phenyl)-1,2,4-oxadiazol-3-yl)pyridin-2(1H)-one

To a solution of 4-(trifluoromethoxy)benzoic acid (17.97 mg, 0.095 mmol) in MeCN (0.500 mL) was added CDI (15.33 mg, 0.095 mmol). The mixture was stirred for 90 minutes at 40° C. and N′-hydroxy-1-(3-(2-methoxyethoxy)benzyl)-6-oxo-1,6-dihydropyridine-3-carboximidamide (30.0 mg, 0.095 mmol) was added. The reaction was stirred at 40° C. for 12 h, DMF (0.500 mL) was added and the reaction was heated at 150° C. for further 3 h. The mixture was diluted with EtOAc (15 mL) and washed three times with water (15 mL). The organic layer was dried over Na₂SO₄, filtered and concentrated under reduced pressure. Purification by SiO₂ gel chromatography (0% to 100% EtOAc/Hexanes) afforded the title compound as a white solid. MS(ES⁺) C₂₄H₂₀F₃N₃O₄ requires: 471. found 472 [M+H]⁺; ¹H NMR (600 MHz, CDCl₃) δ 8.38-8.19 (m, 3H), 8.02 (m, 1H), 7.90-7.75 (m, 2H), 7.25 (m, 1H), 7.04-6.83 (m, 3H), 6.73 (m, 1H), 5.21 (s, 2H), 4.18-4.02 (m, 2H), 3.82-3.66 (m, 2H), 3.44 (s, 3H).

### EXAMPLE 66

### 5-(5-(4-(1,1-Dioxidoisothiazolidin-2-yl)phenyl)-1,2,4-oxadiazol-3-yl)-1-(3-(2-methoxyethoxy)benzyl)pyridin-2(1H)-one

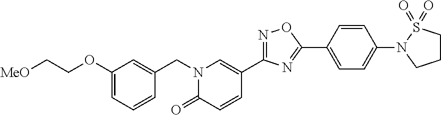 [SMILES: COCCOc1cccc(Cn2cc(-c3noc(-c4ccc(N5CCCS5(=O)=O)cc4)n3)ccc2=O)c1]

To a mixture of Cs₂CO₃ (41 mg, 0.12 mmol), 5-(5-(4-bromophenyl)-1,2,4-oxadiazol-3-yl)-1-(3-(2-methoxyethoxy)benzyl)pyridin-2(1H)-one (synthesized with analogous method to Example 65, using 4-bromobenzoic acid instead of 4-(trifluoromethoxy)benzoic acid; 30 mg, 0.062 mmol), Pd₂(dba)₃ (0.6 mg, 0.62 μmol), and XPhos (1.1 mg, 2.5 μmol) in toluene (311 μL) previously degassed with N₂, was added isothiazolidine 1,1-dioxide (6.4 μl, 0.075 mmol). The mixture was heated to 80° C. for 12 h, cooled to RT, filtered through a pad Celite and concentrated under reduced pressure. The residue was purified by prep-HPLC (Mobile phase: A=0.1% TFA/H₂O, B=0.1% TFA/MeCN; Gradient: B=30%-70% in 12 min; Column: C18) to give the title compound; ¹H NMR (600 MHz, Chloroform-d) δ 8.25 (d, J=2.4 Hz, 1H), 8.13 (d, J=8.8 Hz, 2H), 8.04 (dd, J=9.5, 2.4 Hz, 1H), 7.35 (d, J=8.8 Hz, 2H), 7.28 (m, 1H), 6.95 (d, J=8.0 Hz, 1H), 6.93 (m, 1H), 6.88 (dd, J=8.1, 2.4 Hz, 1H), 6.77 (d, J=9.5 Hz, 1H), 5.22 (s, 2H), 4.14-4.04 (m, 2H), 3.87 (t, J=6.5 Hz, 2H), 3.79-3.68 (m, 2H), 3.46-3.43 (m, 2H), 3.43 (s, 3H), 2.67-2.56 (m, 2H); MS(ES⁺) C₂₆H₂₆N₄O₆S requires: 522. found: 523 [M+H]⁺.

### EXAMPLE 67

### 5-(5-(4-Cyclopropylphenyl)-1,2,4-oxadiazol-3-yl)-1-(3-(2-methoxyethoxy)benzyl)pyridin-2(1H)-one

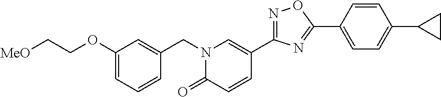 [SMILES: COCCOc1cccc(Cn2cc(-c3noc(-c4ccc(C5CC5)cc4)n3)ccc2=O)c1]

To a mixture of K₃PO₄ (46 mg, 0.22 mmol), 5-(5-(4-bromophenyl)-1,2,4-oxadiazol-3-yl)-1-(3-(2-methoxyethoxy)benzyl)pyridin-2(1H)-one (synthesized in an analogous method to Example 66, 30 mg, 0.062 mmol), Pd(PPh₃)₄ (7.2 mg, 0.62 μmol), and H₂O (31 μL) in toluene (311 μL) previously degassed with N₂ was added cyclopropylboronic acid (7.0 ml, 0.081 mmol). The mixture was heated to 140° C. for 12 h, cooled to RT, filtered through a pad of Celite and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to give the title compound; ¹H NMR (600 MHz, Chloroform-d) δ 8.27 (d, J=2.4 Hz, 1H), 8.07 (dd, J=9.4, 2.4 Hz, 1H), 8.04-7.99 (m, 2H), 7.28 (m, 1H), 7.20 (d, J=8.3 Hz, 2H), 6.97-6.92 (m, 2H), 6.88 (dd, J=8.3, 1.9 Hz, 1H), 6.80 (d, J=9.5 Hz, 1H), 5.22 (s, 2H), 4.14-4.05 (m, 2H), 3.78-3.66 (m, 2H), 3.44 (s, 3H), 2.02-1.93 (m, 1H), 1.15-1.00 (m, 2H), 0.81 (m, 2H); MS(ES⁺) C₂₆H₂₅N₃O₄ requires: 443. found: 444 [M+H]⁺.

### EXAMPLE 68

### 2-(4-(3-(1-(3-(2-Methoxyethoxy)benzyl)-6-oxo-1,6-dihydropyridin-3-yl)-1,2,4-oxadiazol-5-yl)phenyl)-2-methylpropanenitrile

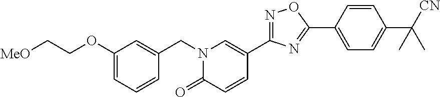 [SMILES: COCCOc1cccc(Cn2cc(-c3noc(-c4ccc(C(C)(C)C#N)cc4)n3)ccc2=O)c1]

To a suspension of sodium hydride (60% in oil, 6.1 mg, 0.15 mmol), in DMF (0.80 mL) at 0° C. was added 2-(4-(3-(1-(3-(2-methoxyethoxy)benzyl)-6-oxo-1,6-dihydropyridin-3-yl)-1,2,4-oxadiazol-5-yl)phenyl)acetonitrile (prepared with analogous method to Example 65 using 4-(cyanomethyl)benzoic acid instead of 4-(trifluoromethoxy)benzoic acid; 30 mg, 0.062 mmol). The mixture was stirred for 30 minutes at 0° C. before the addition of iodomethane (6.5 μL, 0.10 mmol). The mixture was stirred 5 min at 0° C., then warmed to RT and stirred for further 12 h. The reaction mixture was quenched with 1 N HCl (1 mL) and extracted with EtOAc (3×2 mL). The organic layer was washed with H₂O (3×2 mL) and brine (2 mL), dried over Na₂SO₄, filtered and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to give the title compound; ¹H NMR (600 MHz, Chloroform-d) δ 8.26 (d, J=2.4 Hz, 1H), 8.19 (d, J=8.4 Hz, 2H), 8.04 (dd, J=9.5, 2.4 Hz, 1H), 7.67 (d, J=8.4 Hz, 2H), 7.28 (m, 1H), 6.96 (d, J=7.6 Hz, 1H), 6.93 (m, 1H), 6.89 (dd, J=8.3, 2.5 Hz, 1H), 6.77 (d, J=9.5 Hz, 1H), 5.22 (s, 2H), 4.14-4.05 (m, 2H), 3.77-3.69 (m, 2H), 3.44 (s, 3H), 1.78 (s, 6H); MS(ES⁺) C₂₇H₂₆N₄O₄ requires: 470. found: 471 [M+H]⁺.

### EXAMPLE 69

### 1-(3-(3-(Methylsulfonyl)propoxy)benzyl)-5-(5-(4-(trifluoromethoxy)phenyl)-1,2,4-oxadiazol-3-yl)pyridin-2(1H)-one

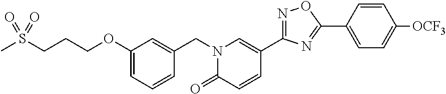 [SMILES: CS(=O)(=O)CCCOc1cccc(Cn2cc(-c3noc(-c4ccc(OC(F)(F)F)cc4)n3)ccc2=O)c1]

### Step 1

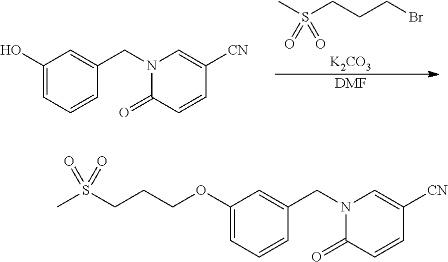

### 1-(3-(3-(Methylsulfonyl)propoxy)benzyl)-6-oxo-1,6-dihydropyridine-3-carbonitrile

To a solution of 1-(3-Hydroxybenzyl)-6-oxo-1,6-dihydropyridine-3-carbonitrile (Example 65, Step 1; 500 mg, 2.21 mmol) in DMF (10 mL) were added 1-bromo-3-(methylsulfonyl)propane (889 mg, 4.42 mmol) and K₂CO₃ (611 mg, 4.42 mmol) The mixture was then heated to 70° C. for 20 h and then diluted with water and extracted with EtOAc. The organic layer was washed with brine, dried over Na₂SO₄, filtered and concentrated under reduced pressure. The residue was purified by SiO₂ gel chromatography (50%-100% EtOAc/Hexanes) to afford the title compound (712 mg, 93%). MS(ES⁺) C₁₇H₁₈N₂O₄S requires: 346. found: 347 [M+H]⁺.

### Step 2

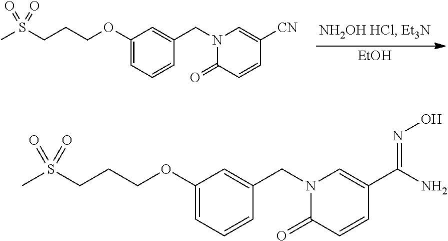 [SMILES: "EtOH" ; CS(=O)(=O)CCCOc1cccc(Cn2cc(C#N)ccc2=O)c1 ; CS(=O)(=O)CCCOc1cccc(Cn2cc(/C(N)=N/O)ccc2=O)c1]

### N′-Hydroxy-1-(3-(3-(methylsulfonyl)propoxy)benzyl)-6-oxo-1,6-dihydropyridine-3-carboximidamide

To a solution of 1-(3-(3-(methylsulfonyl)propoxy)benzyl)-6-oxo-1,6-dihydropyridine-3-carbonitrile (250 mg, 0.722 mmol) and hydroxylamine hydrochloride (55.2 mg, 0.794 mmol) in EtOH (3.00 ml) was added triethylamine (0.111 ml, 0.794 mmol). The mixture was heated at 65° C. for 2 h, cooled to RT, concentrated to a volume of 2 mL and then partitioned between EtOAc (30 mL) and water (30 mL). The aqueous layer was extracted with EtOAc (3×30 mL), the combined organic layers were washed with water (4×30 mL), brine (30 mL), dried over Na₂SO₄, and concentrated under reduced pressure. The residue was dissolved in a minimal amount of DCM and Hexanes resulting in the formation of a white solid percipitate. The mixture was concentrated under reduced pressure to afford the title compound (153 mg, 0.403 mmol, 55.9% yield) as a white solid. MS(ES⁺) C₁₇H₂₁N₃O₅S requires: 379. found 380 [M+H]⁺.

### Step 3

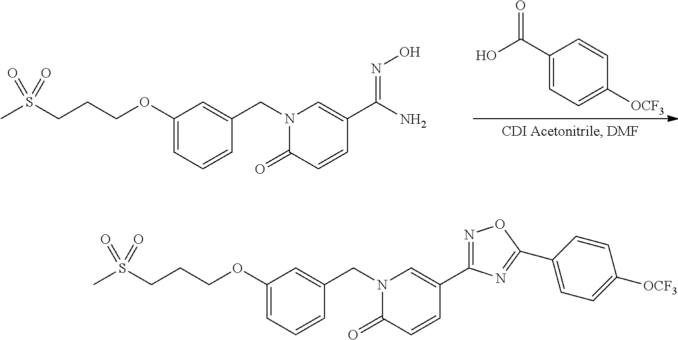 [SMILES: CS(=O)(=O)CCCOc1cccc(Cn2cc(/C(N)=N/O)ccc2=O)c1 ; O=C(O)c1ccc(OC(F)(F)F)cc1 ; CS(=O)(=O)CCCOc1cccc(Cn2cc(-c3noc(-c4ccc(OC(F)(F)F)cc4)n3)ccc2=O)c1]

### 1-(3-(3-(Methylsulfonyl)propoxy)benzyl)-5-(5-(4-(trifluoromethoxy)phenyl)-1,2,4-oxadiazol-3-yl)pyridin-2(1H)-one

Prepared with analogous method to Example 65. Purification by prep-HPLC (Mobile phase: A=0.01% TFA/H₂O, B=0.01% TFA/MeCN; Gradient: B=40%-80% in 12 min; Column: C18) to afford the title compound as a white solid; MS(ES⁺) C₂₅H₂₂F₃N₃O₆S requires: 549. found 550 [M+H]⁺; ¹H NMR (600 MHz, CDCl₃) δ 8.26 (d, J=2.5 Hz, 1H), 8.24-8.19 (m, 2H), 8.02 (dd, J=9.5, 2.5 Hz, 1H), 7.39 (d, J=8.4 Hz, 2H), 7.30 (m, 1H), 6.96 (d, J=7.7 Hz, 1H), 6.90 (m, 1H), 6.83 (dd, J=8.2, 2.3 Hz, 1H), 6.74 (d, J=9.5 Hz, 1H), 5.21 (s, 2H), 4.10 (t, J=5.8 Hz, 2H), 3.24 (t, J=7.7 Hz, 2H), 2.95 (s, 3H), 2.37-2.30 (m, 2H).

### EXAMPLE 70

### 5-(5-(4-(1-Hydroxy-2-methylpropan-2-yl)phenyl)-1,2,4-oxadiazol-3-yl)-1-(3-(2-methoxyethoxy)benzyl)pyridin-2(1H)-one

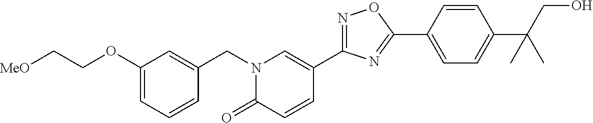 [SMILES: COCCOc1cccc(Cn2cc(-c3noc(-c4ccc(C(C)(C)CO)cc4)n3)ccc2=O)c1]

### Steps 1 to 3:

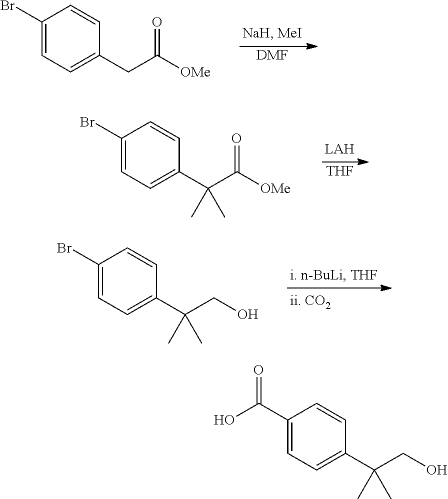

### Step 1: Methyl 2-(4-bromophenyl)-2-methylpropanoate

To a suspension of sodium hydride (60% in oil, 192 mg, 4.80 mmol), in DMF (11 mL) at 0° C. was added methyl 2-(4-bromophenyl)acetate (0.35 mL, 2.2 mmol). The mixture was stirred for 15 minutes at 0° C. and iodomethane (6.5 μL, 0.10 mmol) was added. The mixture was stirred for further 5 minutes at 0° C. and at RT for 12 h, then quenched with 1 N aq. HCl (1 mL) and extracted with EtOAc (3×2 mL). The organic layer washed with H₂O (3×2 mL) and brine (2 mL), dried over Na₂SO₄, filtered and concentrated under reduced pressure. The residue was purified by SiO₂ gel chromatography (0% to 5% EtOAc/Hexanes) to give methyl 2-(4-bromophenyl)-2-methylpropanoate (550 mg, 98%); MS(ES⁺) C₉H₉BrO₂ requires: 228. found: 229/231 [M+H]⁺.

### Step 2: 2-(4-Bromophenyl)-2-methylpropan-1-ol

To a solution of LAH (1.0 M THF, 0.778 mL, 0.778 mmol) in THF (3.9 mL) at 0° C. was added dropwise methyl 2-(4-bromophenyl)-2-methylpropanoate (35 μL, 0.19 mmol). The mixture was stirred 5 minutes at 0° C., warmed to 40° C. for 3 h, then cooled to 0° C. again. H₂O (30 μL) was added slowly with stirring, followed by 1N aq. NaOH (30 uL) and MeOH (90 μL). The mixture was diluted with Et₂O (4 mL) and warmed to RT for 30 minutes. The suspension was filtered through a pad of Celite and concentrated under reduced pressure to give 2-(4-bromophenyl)-2-methylpropan-1-ol as a white solid (34 mg, 76%): MS(ES⁺) C₁₀H₁₃BrO requires: 228. found: 229/230 [M+H]⁺.

### Step 3: 4-(1-Hydroxy-2-methylpropan-2-yl)benzoic acid

To a solution of n-BuLi (1.6 M in Hexane, 0.82 mL, 1.3 mmol) in THF (2.2 mL) at −78° C. was added dropwise 2-(4-bromophenyl)-2-methylpropan-1-ol (100 mg, 0.436 mmol) in THF (0.2 mL). The mixture was stirred at −78° C. for 30 minutes then warmed to −45° C. for 5 minutes. CO₂ was bubbled through the reaction mixture for 1 minute, and the mixture was left under a CO₂ atmosphere, warmed slowly to RT over 20 minutes, then cooled to 0° C. and quenched with 1 N aq. HCl (2 mL). The mixture was diluted with EtOAc (6 mL) and the organic layer was washed with brine (2 mL), dried over Na₂SO₄ and concentrated under reduced pressure. The residue was purified by prep-HPLC (Mobile phase: A=0.1% TFA/H₂O, B=0.1% TFA/MeCN; Gradient: B=20%-50% in 12 min; Column: C18) to give 4-(1-hydroxy-2-methylpropan-2-yl)benzoic acid (12 mg, 14%): MS(ES⁺) C₁₁H₁₄O₃ requires: 194. found: 195 [M+H]⁺.

### Step 4

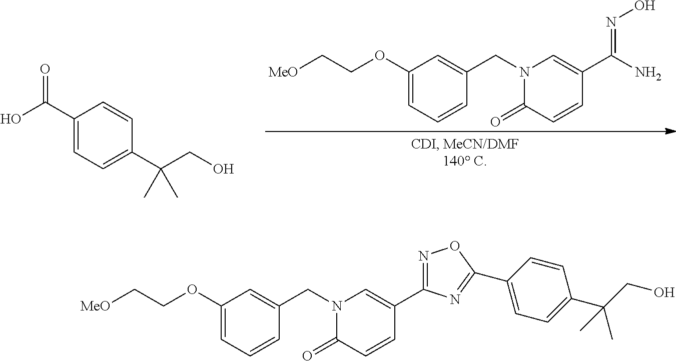 [SMILES: CC(C)(CO)c1ccc(C(=O)O)cc1 ; COCCOc1cccc(Cn2cc(/C(N)=N/O)ccc2=O)c1 ; COCCOc1cccc(Cn2cc(-c3noc(-c4ccc(C(C)(C)CO)cc4)n3)ccc2=O)c1]

Synthesized in an analagous manner to Example 65 using 4-(1-hydroxy-2-methylpropan-2-yl)benzoic acid instead of 4-(trifluoromethoxy)benzoic acid. The residue was purified by prep-HPLC (Mobile phase: A=0.1% TFA/H₂O, B=0.1% TFA/MeCN; Gradient: B=40%-80% in 12 min; Column: C18) to give the title compound; ¹H NMR (600 MHz, Chloroform-d) δ 8.24 (d, J=2.4 Hz, 1H), 8.11 (d, J=8.4 Hz, 2H), 8.02 (dd, J=9.5, 2.4 Hz, 1H), 7.57 (d, J=8.4 Hz, 2H), 7.28 (m, 1H), 6.95 (d, J=8.4 Hz, 1H), 6.94 (m, 1H), 6.88 (dd, J=8.3, 2.5 Hz, 1H), 6.72 (d, J=9.5 Hz, 1H), 5.20 (s, 2H), 4.15-4.05 (m, 2H), 3.76-3.71 (m, 2H), 3.69 (s, 2H), 3.43 (s, 3H), 1.39 (s, 6H); MS(ES⁺) C₂₇H₂₉N₃O₅ requires: 475. found: 476 [M+H]⁺.

### EXAMPLE 71

### 1-(3-(2-Methoxyethoxy)benzyl)-5-(5-(4-(1-(trifluoromethyl)cyclopropyl)phenyl)-1,2,4-oxadiazol-3-yl)pyridin-2(1H)-one

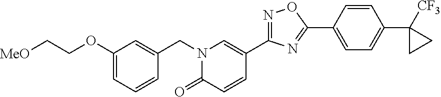 [SMILES: COCCOc1cccc(Cn2cc(-c3noc(-c4ccc(C5(C(F)(F)F)CC5)cc4)n3)ccc2=O)c1]

### Step 1

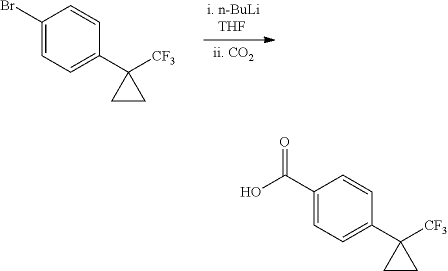 [SMILES: FC(F)(F)C1(c2ccc(Br)cc2)CC1 ; O=C(O)c1ccc(C2(C(F)(F)F)CC2)cc1]

### 4-(1-Hydroxy-2-methylpropan-2-yl)benzoic acid

Prepared in analogous manner to Example 70, step 3; purified by prep-HPLC (Mobile phase: A=0.1% TFA/H₂O, B=0.1% TFA/MeCN; Gradient: B=20%-50% in 12 min; Column: C18) to give 4-(1-hydroxy-2-methylpropan-2-yl)benzoic acid (12 mg, 14%): MS(ES⁺) C₁₁H₁₄O₃ requires: 194. found: 195 [M+H]⁺.

### Step 2

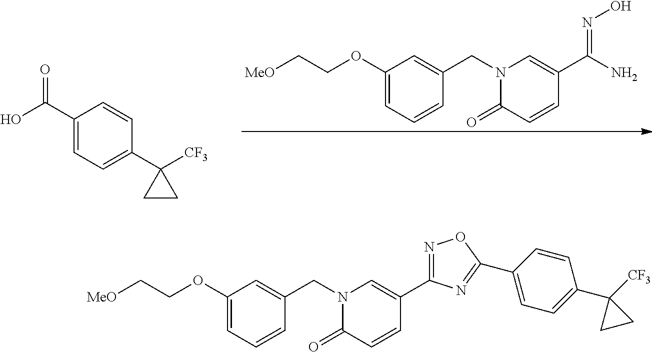 [SMILES: O=C(O)c1ccc(C2(C(F)(F)F)CC2)cc1 ; COCCOc1cccc(Cn2cc(/C(N)=N/O)ccc2=O)c1 ; COCCOc1cccc(Cn2cc(-c3noc(-c4ccc(C5(C(F)(F)F)CC5)cc4)n3)ccc2=O)c1]

### 1-(3-(2-Methoxyethoxy)benzyl)-5-(5-(4-(1-(trifluoromethyl)cyclopropyl)phenyl)-1,2,4-oxadiazol-3-yl)pyridin-2(1H)-one

Synthesized in an analogous method to Example 65, using 4-(1-hydroxy-2-methylpropan-2-yl)benzoic acid instead of 4-(trifluoromethyl)benzoic acid. The residue was purified by prep-HPLC (Mobile phase: A=0.1% TFA/H₂O, B=0.1% TFA/MeCN; Gradient: B=50%-90% in 12 min; Column: C18) to give the title compound; ¹H NMR (600 MHz, Chloroform-d) δ 8.24 (d, J=2.5 Hz, 1H), 8.13 (d, J=8.3 Hz, 2H), 8.02 (dd, J=9.6, 2.5 Hz, 1H), 7.64 (d, J=8.3 Hz, 2H), 7.28 (m, 1H), 6.95 (d, J=8.1 Hz, 1H), 6.93 (m, 1H), 6.88 (dd, J=8.2, 2.5 Hz, 1H), 6.73 (d, J=9.5 Hz, 1H), 5.21 (s, 2H), 4.14-4.06 (m, 2H), 3.78-3.69 (m, 2H), 3.43 (s, 3H), 1.47-1.42 (m, 2H), 1.12-1.07 (m, 2H); MS(ES⁺) C₂₇H₂₄F₃N₃O₄ requires: 511. found: 512 [M+H]⁺.

### EXAMPLE 72

### 15-(5-(4-(1-Methoxy-2-methylpropan-2-yl)phenyl)-1,2,4-oxadiazol-3-yl)-1-(3-(2-methoxyethoxy)benzyl)pyridin-2(1H)-one

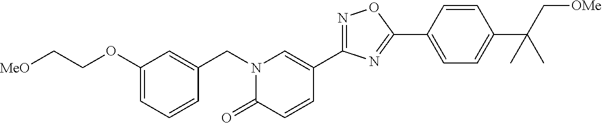 [SMILES: COCCOc1cccc(Cn2cc(-c3noc(-c4ccc(C(C)(C)COC)cc4)n3)ccc2=O)c1]

To a solution of 5-(5-(4-(1-hydroxy-2-methylpropan-2-yl)phenyl)-1,2,4-oxadiazol-3-yl)-1-(3-(2-methoxyethoxy)benzyl)pyridin-2(1H)-one (Example 70, 7.0 mg, 0.015 mmol) in THF (300 μl) at 0° C. was added NaH (60% in mineral oil, 0.9 mg, 0.02 mmol). The resulting mixture was stirred at 0° C. for 30 minutes, iodomethane (2.3 μl, 0.037 mmol) was added and the mixture was stirred for 12 h at RT. The mixture was then quenched with 1 N aq. HCl (1 mL) and extracted with EtOAc (3×1 mL). The combined organic layers were washed with brine (1 mL), dried (Na₂SO₄) and concentrated under reduced pressure. The residue was purified by prep-HPLC (Mobile phase: A=0.1% TFA/H₂O, B=0.1% TFA/MeCN; Gradient: B=50%-90% in 12 min; Column: C18) to give the title compound as a yellow oil; ¹H NMR (600 MHz, Chloroform-d) δ 8.27 (d, J=2.4 Hz, 1H), 8.09 (d, J=8.5 Hz, 2H), 8.06 (dd, J=9.5, 2.5 Hz, 1H), 7.55 (d, J=8.5 Hz, 2H), 7.28 (m, 1H), 6.95 (d, J=7.7 Hz, 1H), 6.94 (m, 1H), 6.89 (dd, J=8.3, 2.5 Hz, 1H), 6.79 (d, J=9.5 Hz, 1H), 5.22 (s, 2H), 4.13-4.09 (m, 2H), 3.75-3.71 (m, 2H), 3.45 (s, 2H), 3.44 (s, 3H), 3.31 (s, 3H), 1.37 (s, 6H); MS (ES⁺) C₂₈H₃₁N₃O₅ requires: 489. found: 490 [M+H]⁺.

### EXAMPLE 73

### 5-(5-(4-(2-Hydroxypropan-2-yl)phenyl)-1,2,4-oxadiazol-3-yl)-1-(3-(2-methoxyethoxy)benzyl)pyridin-2(1H)-one

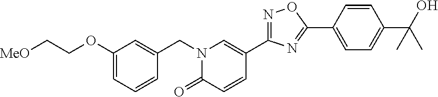 [SMILES: COCCOc1cccc(Cn2cc(-c3noc(-c4ccc(C(C)(C)O)cc4)n3)ccc2=O)c1]

To a solution of 5-(5-(4-acetylphenyl)-1,2,4-oxadiazol-3-yl)-1-(3-(2-methoxyethoxy)benzyl)pyridin-2(1H)-one (prepared in analogous manner to Example 65, using 4-acetylbenzoic acid instead of 4-(trifluoromethoxy)benzoic acid; 15 mg, 0.034 mmol) in THF (350 μl) at 0° C. was added methylmagnesium bromide (56 μl, 0.17 mmol). The mixture was stirred 2 h at 0° C., quenched with 1 N aq. HCl (1 mL) and extracted with EtOAc (3×1 mL). The combined organic layers were dried (Na₂SO₄) and concentrated under reduced pressure. The residue was purified by prep-HPLC (Mobile phase: A=0.1% TFA/H₂O, B=0.1% TFA/MeCN; Gradient: B=30%-70% in 12 min; Column: C18) to give the title compound; ¹H NMR (600 MHz, Chloroform-d) δ 8.25 (d, J=2.4 Hz, 1H), 8.12 (d, J=8.4 Hz, 2H), 8.02 (dd, J=9.6, 2.4 Hz, 1H), 7.67 (d, J=8.4 Hz, 2H), 7.28 (m, 1H), 6.95 (d, J=7.8 Hz, 1H), 6.94 (m, 1H), 6.88 (dd, J=8.3, 2.5 Hz, 1H), 6.73 (d, J=9.5 Hz, 1H), 5.21 (s, 2H), 4.14-4.06 (m, 2H), 3.78-3.70 (m, 2H), 3.43 (s, 3H), 1.63 (s, 6H); MS(ES⁺) C₂₆H₂₇N₃O₅ requires: 461. found: 462 [M+H]⁺.

### EXAMPLE 74

### 2-{[3-(2-Hydroxyethoxy)phenyl]methyl}-6-{3-[4-(trifluoromethoxy)phenyl]-1,2,4-oxadiazol-5-yl}-2,3-dihydropyridazin-3-one

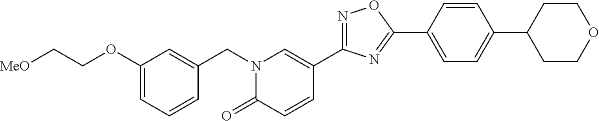 [SMILES: COCCOc1cccc(Cn2cc(-c3noc(-c4ccc(C5CCOCC5)cc4)n3)ccc2=O)c1]

### Step 1 to 3

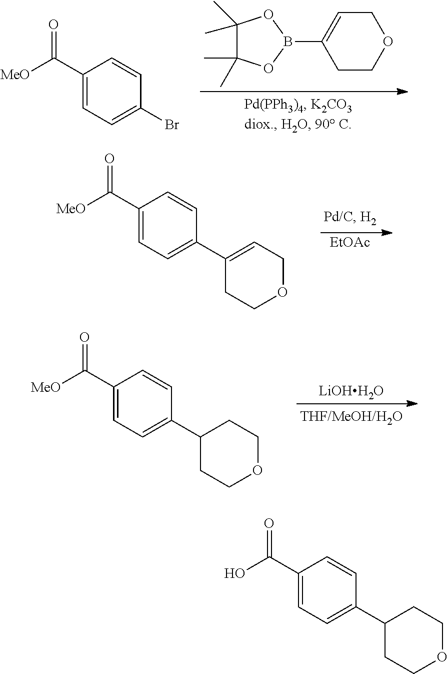

### Step 1: Methyl 4-(3,6-dihydro-2H-pyran-4-yl)benzoate

To a mixture of K₂CO₃ (549 mg, 3.97 mmol), methyl 4-bromobenzoate (388 mg, 1.80 mmol), and Pd₂(PPh₃)₄ (208 mg, 0.180 mmol) in dioxane (15 mL) and H₂O (3.0 mL), previously degassed with N₂, was added 2-(3,6-dihydro-2H-pyran-4-yl)-4,4,5,5-tetramethyl-1,3,2-dioxaborolane (417 mg, 1.99 mmol). The mixture was heated to 90° C. for 12 h, cooled to RT, filtered through a pad of Celite and concentrated under reduced pressure. The residue was purified by SiO₂ gel chromatography (30% to 70% EtOAc/Hexanes) to give methyl 4-(3,6-dihydro-2H-pyran-4-yl)benzoate as a pale yellow solid (357 mg, 91%): MS(ES⁺) C₁₃H₁₄O₃ requires: 218. found: 219 [M+H]⁺.

### Step 2: Methyl 4-(tetrahydro-2H-pyran-4-yl)benzoate

To a solution of 4-(3,6-dihydro-2H-pyran-4-yl)benzoate (1.00 g, 4.58 mmol) in EtOH (23 mL) under a N₂ atmosphere was added 10% Pd/C (488 mg, 0.458 mmol). The mixture was purged with H₂ and stirred for 12 h under a H₂ atmosphere. The mixture was then purged with N₂, filtered through a pad of Celite and concentrated under reduced pressure to give methyl 4-(tetrahydro-2H-pyran-4-yl)benzoate as a white solid (1.00 g, 99%): MS(ES⁺)

C₁₃H₁₆O₃ requires: 220. found: 221 [M+H]⁺.

### Step 3: 4-(Tetrahydro-2H-pyran-4-yl)benzoic acid

To a solution of methyl 4-(tetrahydro-2H-pyran-4-yl)benzoate (1.00 g, 4.54 mmol) in THF/MeOH/H₂O (10 mL, 2.2 mL, 2.2 mL) was added lithium hydroxide hydrate (1.14 g, 27.2 mmol). The mixture was stirred for 72 h at RT and diluted with CH₂Cl₂ (10 mL). The organic layer was washed with 1 N aq. NaOH (3×5 mL), extracted with CH₂Cl₂ (5 mL), and acidified with 6 N aq. HCl until formation of a white precipitate. The aqueous layer was then extracted with CH₂Cl₂ (3×5 mL), the combined organic layers were dried over Na₂SO₄ and concentrated under reduced pressure to give 4-(tetrahydro-2H-pyran-4-yl)benzoic acid as a white solid (700 mg, 75%): MS(ES⁺) C₁₂H₁₄O₃ requires: 206. found: 207 [M+H]⁺.

### Step 4

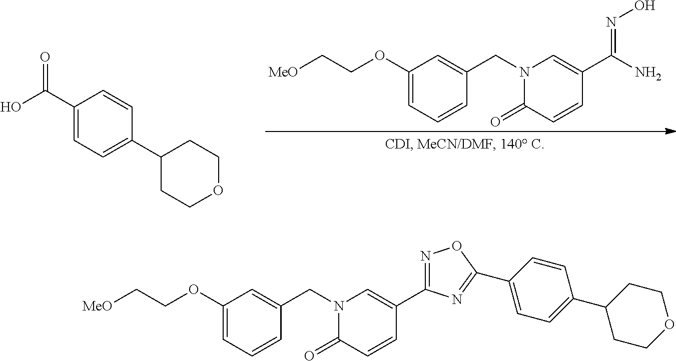 [SMILES: O=C(O)c1ccc(C2CCOCC2)cc1 ; COCCOc1cccc(Cn2cc(/C(N)=N/O)ccc2=O)c1 ; COCCOc1cccc(Cn2cc(-c3noc(-c4ccc(C5CCOCC5)cc4)n3)ccc2=O)c1]

### 1-(3-(2-Methoxyethoxy)benzyl)-5-(5-(4-(tetrahydro-2H-pyran-4-yl)phenyl)-1,2,4-oxadiazol-3-yl)pyridin-2(1H)-one

Synthesized in an analogous manner to Example 65. Purified by prep-HPLC (Mobile phase: A=0.1% TFA/H₂O, B=0.1% TFA/MeCN; Gradient: B=40%-80% in 12 min; Column: C18) to give the title compound; ¹H NMR (600 MHz, Chloroform-d) δ 8.25 (d, J=2.4 Hz, 1H), 8.10 (d, J=8.0 Hz, 2H), 8.02 (dd, J=9.5, 2.4 Hz, 1H), 7.40 (d, J=8.0 Hz, 2H), 7.28 (m, 1H), 6.98-6.92 (m, 2H), 6.88 (m, 1H), 6.73 (d, J=9.5 Hz, 1H), 5.21 (s, 2H), 4.15-4.07 (m, 4H), 3.74 (dd, J=5.5, 3.8 Hz, 2H), 3.60-3.49 (m, 2H), 3.44 (s, 3H), 2.86 (m, 1H), 1.91-1.77 (m, 4H); MS(ES⁺) C₂₈H₂₉N₃O₅ requires: 487. found: 488 [M+H]⁺.

### EXAMPLE 75

### 1-(3-(2-Methoxyethoxy)benzyl)-5-(5-(4-(tetrahydro-2H-thiopyran-4-yl)phenyl)-1,2,4-oxadiazol-3-yl)pyridin-2(1H)-one

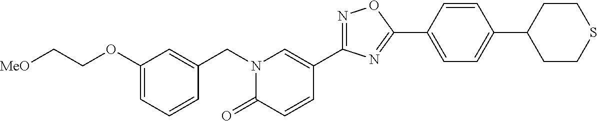 [SMILES: COCCOc1cccc(Cn2cc(-c3noc(-c4ccc(C5CCSCC5)cc4)n3)ccc2=O)c1]

To a solution of 5-(5-(4-(3,6-dihydro-2H-thiopyran-4-yl)phenyl)-1,2,4-oxadiazol-3-yl)-1-(3-(2-methoxyethoxy)benzyl)pyridin-2(1H)-one (synthesized in an analogous manner to Example 74; 10 mg, 0.020 mmol) in EtOAc (2 mL) under N₂ atmosphere was added 10% Pd/C (4.2 mg, 4.0 μmol). The mixture was purged with H₂ and stirred for 12 h under a H₂ atmosphere. The mixture was then purged with N₂, filtered through a pad of Celite and concentrated under reduced pressure. The residue was purified by prep-HPLC (Mobile phase: A=0.1% TFA/H₂O, B=0.1% TFA/MeCN; Gradient: B=50%-90% in 12 min; Column: C18) to give the title compound; MS(ES⁺) C₂₈H₂₉N₃O₄S requires: 503. found: 504 [M+H]⁺; ¹H NMR (600 MHz, Methanol-d₄) δ ¹H NMR (600 MHz, Chloroform-d) δ 8.24 (d, J=2.4 Hz, 1H), 8.09 (d, J=8.3 Hz, 2H), 8.02 (dd, J=9.5, 2.4 Hz, 1H), 7.37 (d, J=8.3 Hz, 2H), 7.27 (m, 1H), 6.96-6.92 (m, 2H), 6.88 (dd, J=8.2, 2.2 Hz, 1H), 6.73 (d, J=9.5 Hz, 1H), 5.20 (s, 2H), 4.16-4.06 (m, 2H), 3.77-3.66 (m, 1H), 3.43 (s, 3H), 2.92-2.80 (m, 2H), 2.73 (bd, J=14.1 Hz, 2H), 2.68-2.57 (m, 2H), 2.17 (dd, J=13.6, 2.8 Hz, 2H), 1.97-1.82 (m, 2H).

### EXAMPLE 76

### 1-(3-(2-Hydroxypropan-2-yl)benzyl)-5-(5-(4-isopropoxyphenyl)-1,2,4-oxadiazol-3-yl)pyridin-2(1H)-one

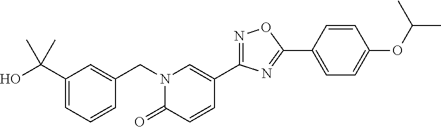 [SMILES: CC(C)Oc1ccc(-c2nc(-c3ccc(=O)n(Cc4cccc(C(C)(C)O)c4)c3)no2)cc1]

### Step 1

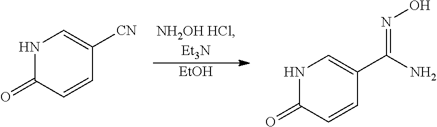

### N′-Hydroxy-6-oxo-1,6-dihydropyridine-3-carboximidamide

To a solution of 6-oxo-1,6-dihydropyridine-3-carbonitrile (1.00 g, 8.33 mmol) and hydroxylamine hydrochloride (1.157 g, 16.65 mmol) in EtOH (10 ml) was added triethylamine (2.32 ml, 16.65 mmol). The mixture was heated at 65° C. for 3 h, cooled to RT and allowed to sit at this temperature overnight. The precipitate was collected by filtration and the solid was dried on the lyophilizer to afford N′-hydroxy-6-oxo-1,6-dihydropyridine-3-carboximidamide (1.083 g, 7.07 mmol, 85% yield) as a white solid.

### Step 2

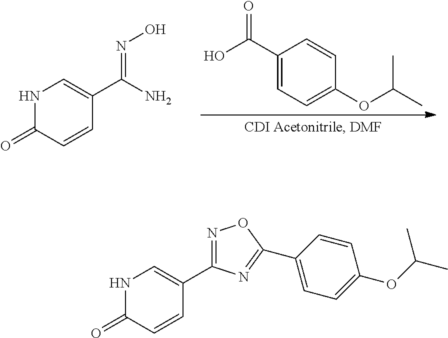 [SMILES: N/C(=N\O)c1ccc(=O)[nH]c1 ; CC(C)Oc1ccc(C(=O)O)cc1 ; CC(C)Oc1ccc(-c2nc(-c3ccc(=O)[nH]c3)no2)cc1]

### 5-(5-(4-Isopropoxyphenyl)-1,2,4-oxadiazol-3-yl)pyridin-2(1H)-one

To a solution of 4-isopropoxybenzoic acid (588 mg, 3.27 mmol) in MeCN (12.60 mL) was added CDI (635 mg, 3.92 mmol). The reaction was stirred for 90 minutes at 40° C. and then N′-hydroxy-6-oxo-1,6-dihydropyridine-3-carboximidamide (500 mg, 3.27 mmol) was added. The reaction was stirred for further 16 h at 40° C., DMF (12.60 mL) was then added and the reaction was heated at 150° C. for 3 h. The reaction was then diluted with EtOAc (15 mL) and washed with water (3×15 mL). The organic layer was dried over Na₂SO₄, filtered and concentrated under reduced pressure. The residue was purified by SiO₂ gel chromatography (0%-10% MeOH/DCM) to afford the title compound (280 mg, 0.942 mmol, 28.8% yield) as a white solid. MS(ES⁺) C₁₆H₁₅N₃O₃ requires: 297. found 298 [M+H]⁺.

### Step 3

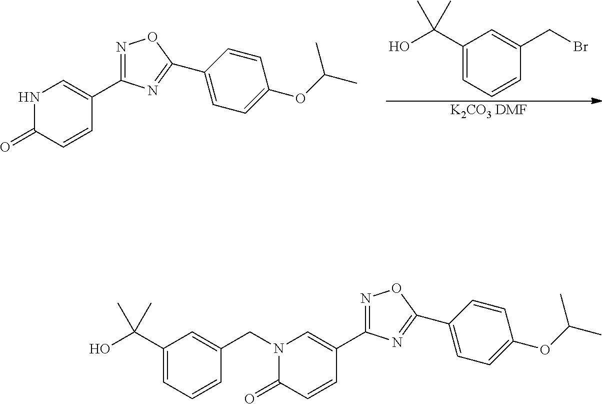

### 1-(3-(2-Hydroxypropan-2-yl)benzyl)-5-(5-(4-isopropoxyphenyl)-1,2,4-oxadiazol-3-yl)pyridin-2(1H)-one

To a solution of 5-(5-(4-Isopropoxyphenyl)-1,2,4-oxadiazol-3-yl)pyridin-2(1H)-one (30.0 mg, 0.101 mmol) in DMF (1.0 ml) was added K₂CO₃ (27.9 mg, 0.202 mmol). The suspension was stirred at RT for 5 minutes and then 2-(3-(bromomethyl)phenyl)propan-2-ol (Example 39, Step 1; 25.4 mg, 0.111 mmol) was added. The reaction was heated at 65° C. for 4 h, diluted with EtOAc (15 mL) and washed with water (3×15 mL). The organic layer was dried over Na₂SO₄, filtered and concentrated under reduced pressure. The residue was purified by prep-HPLC (Mobile phase: A=0.01% TFA/H₂O, B=0.01% TFA/MeCN; Gradient: B=40%-80% in 12 min; Column: C18) to afford the title compound as a white solid. MS (ES⁺) C₂₆H₂₇N₃O₄ requires: 445. found 446 [M+H]⁺ and 428 [M-OH]⁺; ¹H NMR (600 MHz, CDCl₃) δ 8.26 (d, J=2.0 Hz, 1H), 8.06 (d, J=8.6 Hz, 2H), 8.01 (dd, J=9.5, 2.0 Hz, 1H), 7.54 (s, 1H), 7.43 (d, J=7.7 Hz, 1H), 7.34 (m, 1H), 7.21 (d, J=7.7 Hz, 1H), 6.98 (d, J=8.6 Hz, 2H), 6.72 (d, J=9.5 Hz, 1H), 5.25 (s, 2H), 4.67 (septuplet, J=6.0 Hz, 1H), 1.58 (s, 6H), 1.38 (d, J=6.0 Hz, 6H).

### EXAMPLE 77

### 1-(3-(4-(Methylsulfonyl)piperidin-1-yl)benzyl)-5-(5-(4-morpholinophenyl)-1,2,4-oxadiazol-3-yl)pyridin-2(1H)-one

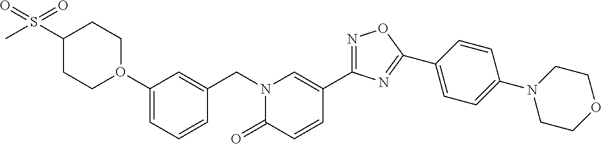

### Step 1

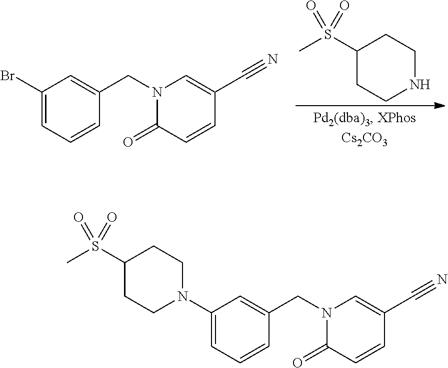

### 1-(3-(4-(Methylsulfonyl)piperidin-1-yl)benzyl)-6-oxo-1,6-dihydropyridine-3-carbonitrile

Synthesized with an analogous manner to Example 31, Step 2 using 1-(3-bromobenzyl)-6-oxo-1,6-dihydropyridine-3-carbonitrile (Example 64, Step 1); 450 mg, 35%. MS(ES⁺) C₁₉H₂₁N₃O₃S requires: 371. found: 372 [M+H]⁺.

### Step 2

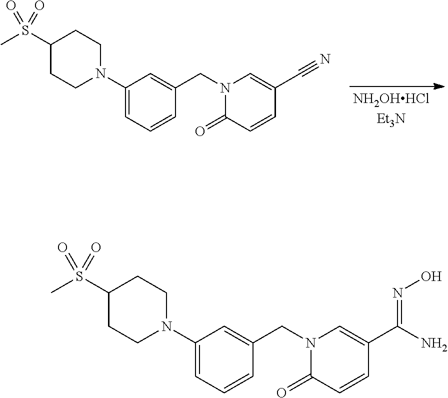 [SMILES: CS(=O)(=O)C1CCN(c2cccc(Cn3cc(C#N)ccc3=O)c2)CC1 ; CS(=O)(=O)C1CCN(c2cccc(Cn3cc(/C(N)=N/O)ccc3=O)c2)CC1]

### (Z)—N′-Hydroxy-1-(3-(4-(methylsulfonyl)piperidin-1-yl)benzyl)-6-oxo-1,6-dihydropyridine-3-carboximidamide

To a solution of 1-(3-(4-(methylsulfonyl)piperidin-1-yl)benzyl)-6-oxo-1,6-dihydropyridine-3-carbonitrile (200 mg, 0.538 mmol) in EtOH (2 mL) were added hydroxylamine hydrochloride (41 mg, 0.592 mmol) and triethylamine (0.083 mL, 0.592 mmol). The mixture was heated to 65° C. for 2 h, cooled to RT, concentrated under reduced pressure and partitioned between EtOAc and water. The aqueous layer was extracted with EtOAc, the combined organic layers were washed with water and brine, dried over Na₂SO₄ and concentrated under reduced pressure. The residue was dissolved in a minimal amount of DCM and Hexanes resulting in the formation of a white solid percipitate. The mixture was concentrated under reduced pressure and then lyophilized to afford the title compound (41 mg, 19%). MS(ES⁺) C₁₉H₂₄N₄O₄S requires: 404. found: 405 [M+H]⁺.

### Step 3

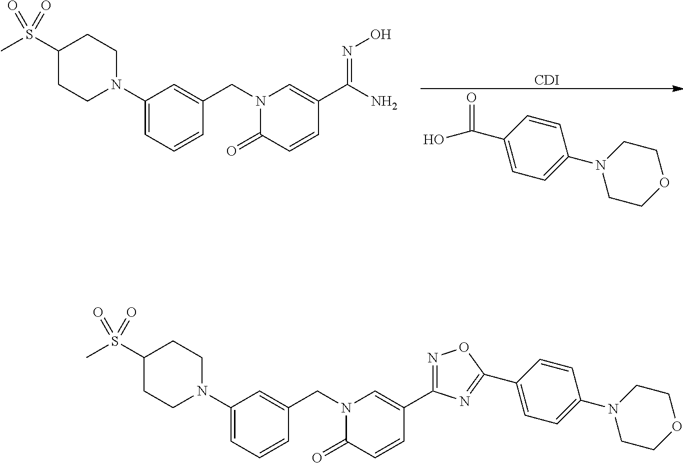

### 1-(3-(4-(Hethylsulfonyl)piperidin-1-yl)benzyl)-5-(5-(4-morpholinophenyl)-1,2,4-oxadiazol-3-yl)pyridin-2(1H)-one

Synthesized in an analogous manner to Example 65; MS(ES⁺) C₃₀H₃₃N₅O₅S requires: 575. found: 576 [M+H]⁺; ¹H NMR (600 MHz, CDCl₃) δ 8.32 (s, 1H), 8.11 (d, J=9.6 Hz, 1H), 8.03 (d, J=9.0 Hz, 2H), 7.35 (m, 1H), 7.23 (s, 1H), 7.12 (d, J=8.4 Hz, 1H), 7.07 (d, J=7.8 Hz, 1H), 6.96 (d, J=9.0 Hz, 2H), 6.83 (d, J=9.6 Hz, 1H), 5.24 (s, 2H), 3.88 (m, 6H), 3.34 (m, 4H), 3.06 (m, 1H), 3.01 (m, 2H), 2.89 (s, 3H), 2.38 (m, 2H), 2.17 (m, 2H).

### EXAMPLE 78

### 5-(5-(4-Isopropoxyphenyl)-1,2,4-oxadiazol-3-yl)-1-(3-(4-(methylsulfonyl)piperidin-1-yl)benzyl)pyridin-2(1H)-one

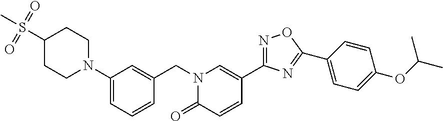 [SMILES: CC(C)Oc1ccc(-c2nc(-c3ccc(=O)n(Cc4cccc(N5CCC(S(C)(=O)=O)CC5)c4)c3)no2)cc1]

To a solution of 5-(5-(4-Isopropoxyphenyl)-1,2,4-oxadiazol-3-yl)pyridin-2(1H)-one (Example 76, Step 2, 40 mg, 0.135 mmol) in DMF (1.0 ml) was added Cs₂CO₃ (175 mg, 0.538 mmol). The reaction was stirred at RT for 5 minutes and then 1-(3-(chloromethyl)phenyl)-4-(methylsulfonyl)piperidine (Example 105, Step 4, 42 mg, 0.148 mmol) was added. The mixture was heated at 65° C. for 4 h, diluted with EtOAc (15 mL) and washed with water (3×15 mL). The organic layer was dried over Na₂SO₄, filtered and concentrated under reduced pressure. The residue was purified by prep-HPLC (Mobile phase: A=0.01% TFA/H₂O, B=0.01% TFA/MeCN; Gradient: B=40%-80% in 12 min; Column: C18) to afford the title compound as a white solid. MS(ES⁺) C₂₉H₃₂N₄O₅S requires: 548. found 549 [M+H]⁺. ¹H NMR (600 MHz, CDCl₃) δ 8.26 (d, J=2.3 Hz, 1H), 8.09-8.04 (m, 2H), 8.02 (dd, J=9.5, 2.3 Hz, 1H), 7.27 (m, 1H), 7.03-6.97 (m, 3H), 6.91 (dd, J=8.1, 2.1 Hz, 1H), 6.88 (d, J=7.6 Hz, 1H), 6.74 (d, J=9.5 Hz, 1H), 5.19 (s, 2H), 4.67 (septuplet, J=6.0 Hz, 1H), 3.88-3.82 (m, 2H), 3.00-2.93 (m, 1H), 2.86 (s, 3H), 2.84-2.77 (m, 2H), 2.30-2.23 (m, 2H), 2.04-1.94 (m, 2H), 1.38 (d, J=6.0 Hz, 6H).

### EXAMPLE 79

### 1-(3-(2-Methoxyethoxy)benzyl)-5-(5-(4-(tetrahydro-2H-pyran-4-yl)phenyl)-1,2,4-oxadiazol-3-yl)pyridin-2(1H)-one

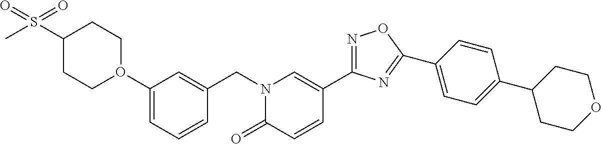

Synthesized from 4-(tetrahydro-2H-pyran-4-yl)benzoic acid and N′-hydroxy-1-(3-(4-(methylsulfonyl)piperidin-1-yl)benzyl)-6-oxo-1,6-dihydropyridine-3-carboximidamide in a manner analogous to Example 77; ¹H NMR (600 MHz, Chloroform-d) δ 8.26 (d, J=2.4 Hz, 1H), 8.12-8.07 (m, 2H), 8.01 (dd, J=9.5, 2.5 Hz, 1H), 7.40 (d, J=8.3 Hz, 2H), 7.25 (m, 1H), 6.97 (m, 1H), 6.91-6.83 (m, 2H), 6.72 (d, J=9.5 Hz, 1H), 5.19 (s, 2H), 4.15-4.07 (m, 2H), 3.87-3.80 (m, 2H), 3.55 (m, 2H), 2.95 (m, 1H), 2.87 (d, J=16.2 Hz, 4H), 2.78 (m, 2H), 2.27-2.21 (m, 2H), 2.00-1.91 (m, 2H), 1.90-1.78 (m, 4H); MS(ES⁺) C₃₁H₃₄N₄O₅S requires: 574. found: 575 [M+H]⁺.

### EXAMPLE 80

### 5-(5-(4-(1-Hydroxycyclohexyl)phenyl)-1,2,4-oxadiazol-3-yl)-1-(3-(2-methoxyethoxy)benzyl)pyridin-2(1H)-one

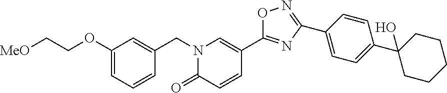 [SMILES: COCCOc1cccc(Cn2cc(-c3nc(-c4ccc(C5(O)CCCCC5)cc4)no3)ccc2=O)c1]

### Steps 1 to 3

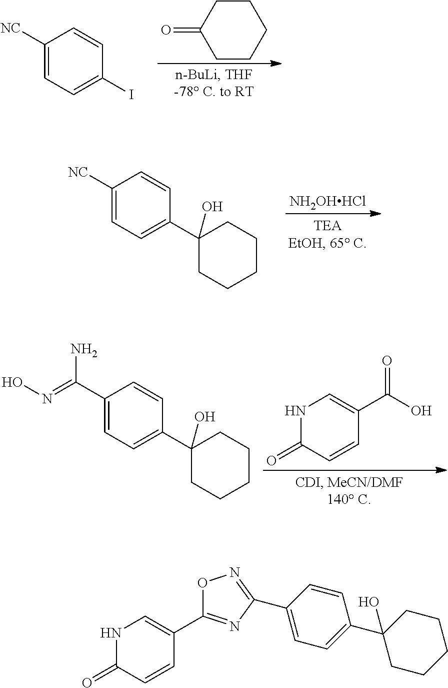

### Step 1: 4-(1-Hydroxycyclohexyl)benzonitrile

To a solution of 4-iodobenzonitrile (100 mg, 0.437 mmol) in THF (4.4 mL) at −78° C. was added n-BuLi (1.6 M in Hexane, 0.27 mL, 0.44 mmol). The mixture was stirred for 10 min at −78° C., before the addition of cyclohexanone (0.091 mL, 0.873 mmol) and then was allowed to slowly warm to RT for 1 h. 1 N aq. HCl (2 mL) was added and the mixture was extracted with EtOAc (3×3 mL). The combined organic layers were washed with brine (2 mL), dried over Na₂SO₄, and concentrated under reduced pressure. The residue was purified by SiO₂ gel chromatography (10% to 70% EtOAc/Hexanes) to give 4-(1-hydroxycyclohexyl)benzonitrile (50 mg, 57%): MS(ES⁺) C₁₃H₁₅NO requires: 201. found: 202 [M+H]⁺.

### Step 2: N′-Hydroxy-4-(1-hydroxycyclohexyl)benzimidamide

To a suspension of 4-(1-hydroxycyclohexyl)benzonitrile (50 mg, 0.25 mmol) and hydroxylamine hydrochloride (35 mg, 0.50 mmol) in EtOH (1.6 mL) was added Et₃N (69 μL, 0.50 mmol). The mixture as heated to 80° C. for 3 h, the cooled to RT and diluted with H₂O (3 mL). The mixture was extracted with EtOAc (3×3 mL), the combined organic layers were washed with H₂O (2 mL), brine (2 mL), dried (Na₂SO₄) and concentrated under reduced pressure to give N′-hydroxy-4-(1-hydroxycyclohexyl)benzimidamide as a white solid (56 mg, 98%): MS(ES⁺) C₁₃H₁₈N₂O₂ requires: 234. found: 235 [M+H]⁺.

### Step 3: 5-(3-(4-(1-Hydroxycyclohexyl)phenyl)-1,2,4-oxadiazol-5-yl)pyridin-2(1H)-one

Synthesized in an analogous method to Example 76, Step 2; MS (ES⁺) C₁₉H₁₉N₃O₃ requires: 337. found: 337 [M+H]⁺.

### Step 4

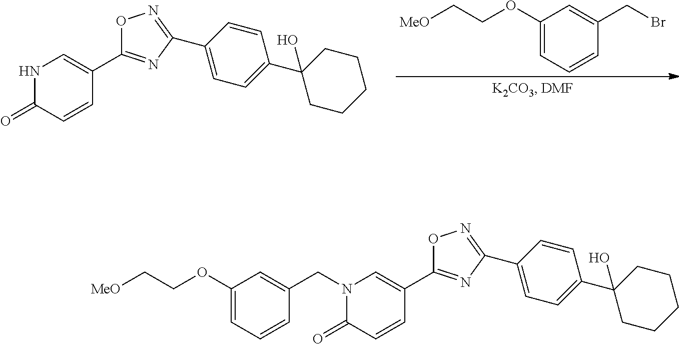 [SMILES: O=c1ccc(-c2nc(-c3ccc(C4(O)CCCCC4)cc3)no2)c[nH]1 ; COCCOc1cccc(CBr)c1 ; COCCOc1cccc(Cn2cc(-c3nc(-c4ccc(C5(O)CCCCC5)cc4)no3)ccc2=O)c1]

### 5-(3-(4-(1-Hydroxycyclohexyl)phenyl)-1,2,4-oxadiazol-5-yl)-1-(3-(2-methoxyethoxy)benzyl)pyridin-2(1H)-one

Synthesized in an analogous method to Example 26. Purified by prep-HPLC (Mobile phase: A=0.1% TFA/H₂O, B=0.1% TFA/MeCN; Gradient: B=40%-80% in 12 min; Column: C18) to give the title compound; ¹H NMR (500 MHz, DMSO-d₆) δ 8.97 (d, J=2.5 Hz, 1H), 8.06 (dd, J=9.6, 2.5 Hz, 1H), 7.99 (d, J=8.1 Hz, 2H), 7.69 (d, J=8.4 Hz, 2H), 7.27 (m, 1H), 6.98-6.84 (m, 3H), 6.65 (d, J=9.5 Hz, 1H), 5.24 (s, 2H), 4.10-4.01 (m, 2H), 3.68-3.60 (m, 2H), 3.28 (s, 3H), 1.85-1.44 (m, 10H), 1.27 (ddd, J=18.0, 13.1, 8.9 Hz, 1H); MS (ES⁺) C₂₉H₃₁N₃O₅ requires: 501. found: 502 [M+H]⁺.

### EXAMPLE 81

### 1-(3-(2-Methoxyethoxy)benzyl)-5-(5-(4-(2-methoxypropan-2-yl)phenyl)-1,2,4-oxadiazol-3-yl)pyridin-2(1H)-one

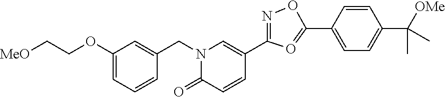

To a solution of NaH (1.3 mg, 0.033 mmol) in THF (0.5 mL) at 0° C. was added 5-(5-(4-(2-hydroxypropan-2-yl)phenyl)-1,2,4-oxadiazol-3-yl)-1-(3-(2-methoxyethoxy)benzyl)pyridin-2(1H)-one (Example 73; 10 mg, 0.022 mmol). The mixture was stirred 15 minutes at 0° C., iodomethane (4.1 μL, 0.065 mmol) was added and the resulting mixture was stirred for 1 h at 0° C. and at RT for 6 h. The mixture was quenched with 1 N aq. HCl (1 mL) and extracted with EtOAc (3×1 mL). The organic layer was washed with brine (1 mL), dried over Na₂SO₄, and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to give the title compound; ¹H NMR (600 MHz, Chloroform-d) δ 8.27 (m, 1H), 8.15-8.10 (m, 2H), 8.05 (dd, J=9.5, 2.4 Hz, 1H), 7.61-7.57 (m, 2H), 7.28 (m, 1H), 6.98-6.92 (m, 2H), 6.91-6.86 (m, 1H), 6.77 (m, 1H), 5.22 (s, 2H), 4.15-4.05 (m, 2H), 3.76-3.71 (m, 2H), 3.44 (s, 3H), 3.12 (s, 3H), 1.57 (s, 6H); MS(ES⁺) C₂₇H₂₉N₃O₅ requires: 475. found: 476 [M+H]⁺.

### EXAMPLE 82

### 6-(3-(4-(Difluoromethoxy)phenyl)-1,2,4-oxadiazol-5-yl)-2-(3-(4-(methylsulfonyl)piperidin-1-yl)benzyl)pyridazin-3(2H)-one

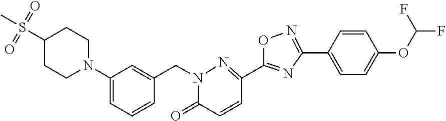 [SMILES: CS(=O)(=O)C1CCN(c2cccc(Cn3nc(-c4nc(-c5ccc(OC(F)F)cc5)no4)ccc3=O)c2)CC1]

### Step 1

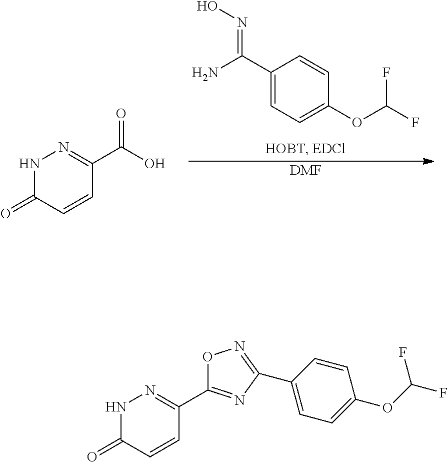

### 6-(3-(4-(Difluoromethoxy)phenyl)-1,2,4-oxadiazol-5-yl)pyridazin-3(2H)-one

To a solution of 1,6-dihydro-6-oxo-3-pyridazinecarboxylic acid mono-hydrate (76 mg, 0.54 mmol) in DMF (2.5 mL) was added 4-(difluoromethoxy)-N′-hydroxybenzimidamide (100 mg, 0.49 mmol), followed by EDC.HCl (114 mg, 0.59 mmol) and HOBT (91 mg, 0.59 mmol). The mixture was stirred at RT for 60 minutes, then heated to 140° C. for 2 h. The reaction mixture was cooled to RT, diluted with H₂O (5 mL) and extracted with EtOAc (4×10 mL). The combined organic layers were washed with H₂O (15 mL) and brine (15 mL), dried over Na₂SO₄, filtered, and concentrated under reduced pressure. The residue was purified prep-HPLC (Mobile phase: A=0.1% TFA/H₂O, B=0.1% TFA/MeCN; Gradient: B=20%-50% in 12 min; Column: C18) to give the title compound as a white solid (26 mg, 17%). MS (ES⁺) C₁₃H₈F₂N₄O₃ requires: 306. found: 307 [M+H]⁺.

### Step 2

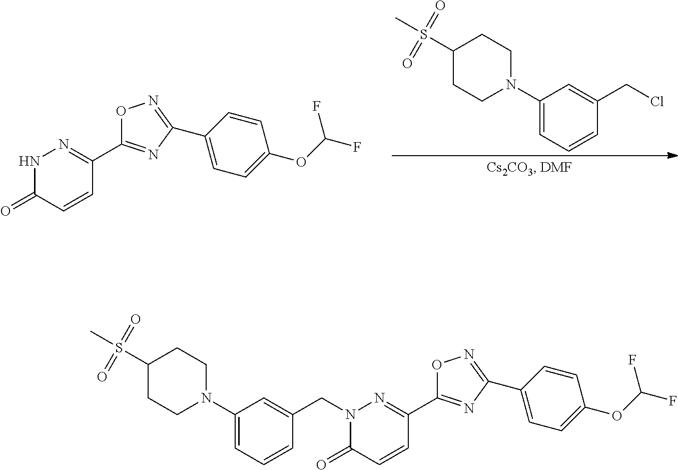 [SMILES: O=c1ccc(-c2nc(-c3ccc(OC(F)F)cc3)no2)n[nH]1 ; CS(=O)(=O)C1CCN(c2cccc(CCl)c2)CC1 ; CS(=O)(=O)C1CCN(c2cccc(Cn3nc(-c4nc(-c5ccc(OC(F)F)cc5)no4)ccc3=O)c2)CC1]

### 6-(3-(4-(Difluoromethoxy)phenyl)-1,2,4-oxadiazol-5-yl)-2-(3-(4-(methylsulfonyl)piperidin-1-yl)benzyl)pyridazin-3(2H)-one

To a suspension of 6-(3-(4-(difluoromethoxy)phenyl)-1,2,4-oxadiazol-5-yl)pyridazin-3(2H)-one (26 mg, 0.085 mmol) in DMF (1.4 mL), were added Cs₂CO₃ (97 mg, 0.29 mmol), and 1-(3-(chloromethyl)phenyl)-4-(methylsulfonyl)piperidine (Example 105, Step 4, 36.7 mg, 0.12 mmol). The mixture was stirred at RT for 18 h and then filtered through a pad of Celite. The filtrate was diluted with H₂O (10 mL) and extracted with EtOAc (3×10 mL). The combined organic layers were washed with brine (2×15 mL), dried over Na₂SO₄, filtered, and concentrated under reduced pressure. The residue was purified by prep-HPLC (Mobile phase: A=0.1% TFA/H₂O, B=0.1% TFA/MeCN; Gradient: B=30%-70% in 12 min; Column: C18) to give the title compound as a white solid; MS (ES⁺) C₂₆H₂₅F₂N₅O₅S requires: 557. found: 558 [M+H]⁺; ¹H NMR (600 MHz, DMSO-d₆) δ 8.17-8.08 (m, 3H), 7.53-7.27 (m, 3H), 7.25-7.18 (m, 2H), 7.03 (m, 1H), 6.94 (m, 1H), 6.75 (d, J=7.5 Hz, 1H), 5.36 (s, 2H), 3.84 (m, 2H), 3.28 (m, 1H), 2.95 (s, 3H), 2.78 (m, 2H), 2.13-2.04 (m, 2H), 1.69 (m, 2H).

### EXAMPLE 83

### 2-(3-(4-(Methylsulfonyl)piperidin-1-yl)benzyl)-6-(3-(4-morpholinophenyl)-1,2,4-oxadiazol-5-yl)pyridazin-3(2H)-one

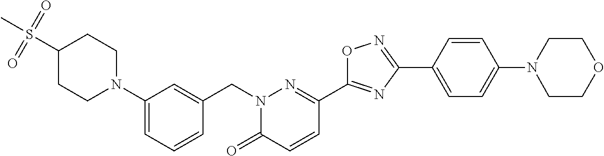 [SMILES: CS(=O)(=O)C1CCN(c2cccc(Cn3nc(-c4nc(-c5ccc(N6CCOCC6)cc5)no4)ccc3=O)c2)CC1]

Synthetized in analogous manner to Example 82 using N′-hydroxy-4-morpholinobenzimidamide instead of 4-(difluoromethoxy)-N′-hydroxybenzimidamide. The crude product was purified by prep-HPLC (Mobile phase: A=0.1% TFA/H₂O, B=0.1% TFA/MeCN; Gradient: B=30%-70% in 12 min; Column: C18) to give the title compound as an off-white solid; MS(ES⁺) C₂₉H₃₂N₆O₅S requires: 576. found: 577 [M+H]⁺; ¹H NMR (600 MHz, DMSO-d₆) δ 8.12 (d, J=9.7 Hz, 1H), 7.94-7.90 (m, 2H), 7.23-7.17 (m, 2H), 7.13-7.09 (m, 2H), 7.02 (t, J=2.0 Hz, 1H), 6.92 (dd, J=8.4, 2.5 Hz, 1H), 6.72 (d, J=7.5 Hz, 1H), 5.35 (s, 2H), 3.84 (m, 2H), 3.75 (m, 4H), 3.29-3.24 (m, 5H), 2.94 (s, 3H), 2.75 (m, 2H), 2.11-2.04 (m, 2H), 1.68 (m, 2H).

### EXAMPLE 84

### 5-(5-(4-Cyclohexylphenyl)-1,2,4-oxadiazol-3-yl)-1-(3-(2-methoxyethoxy)benzyl)pyridin-2(1H)-one

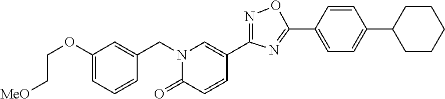 [SMILES: COCCOc1cccc(Cn2cc(-c3noc(-c4ccc(C5CCCCC5)cc4)n3)ccc2=O)c1]

To a mixture of 4-cyclohexylbenzoic acid (65 mg, 0.315 mmol), 3-propanediamine,n′-(ethylcarbonimidoyl)-n,n-dimethylmonhydrochloride (90 mg, 0.473 mmol) and 1-hydroxybenzotriazole (64 mg, 0.473 mmol) in DMF (5 mL) was added (Z)—N′-hydroxy-1-(3-(2-methoxyethoxy)benzyl)-6-oxo-1,6-dihydropyridine-3-carboximidamide (Example 65, Step 3; 100 mg, 0.315 mmol). The mixture was stirred at 100° C. for 16 h, then cooled to RT and purified directly by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to give the title compound as a white solid; MS(ES⁺) C₂₉H₃₁N₃O₄ requires: 485. found: 486 [M+H]⁺; ¹H NMR (500 MHz, CDCl₃) δ 8.25 (d, J=2.4 Hz, 1H), 8.07 (d, J=8.3 Hz, 2H), 8.03 (s, 1H), 7.37 (d, J=8.3 Hz, 2H), 7.28 (m, 1H), 6.94 (m, 2H), 6.88 (dd, J=8.2, 2.2 Hz, 1H), 6.72 (d, J=9.5 Hz, 1H), 5.20 (s, 2H), 4.11 (m, 2H), 3.74 (m, 2H), 3.44 (s, 3H), 2.59 (m, 1H), 1.88 (m, 4H), 1.78 (bd, J=12.9 Hz, 1H), 1.35 (m, 4H), 1.25 (m, 1H).

### EXAMPLE 85

### 2-(4-(3-(1-(3-(2-Methoxyethoxy)benzyl)-6-oxo-1,6-dihydropyridin-3-yl)-1,2,4-oxadiazol-5-yl)phenylsulfonyl)acetonitrile

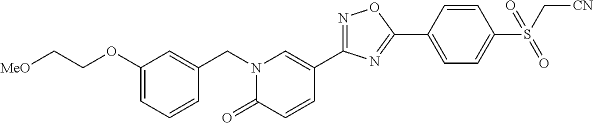 [SMILES: COCCOc1cccc(Cn2cc(-c3noc(-c4ccc(S(=O)(=O)CC#N)cc4)n3)ccc2=O)c1]

### Steps 1 to 4

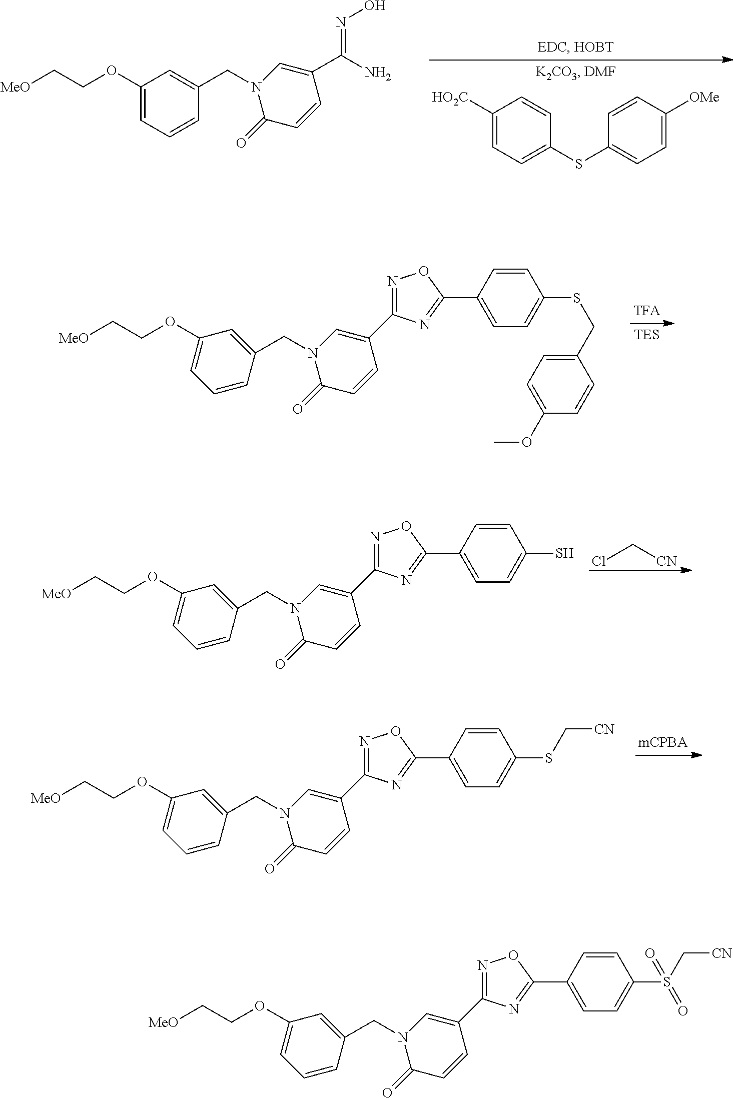

### Step 1: 5-(5-(4-(4-Methoxybenzylthio)phenyl)-1,2,4-oxadiazol-3-yl)-1-(3-(2-methoxy ethoxy)benzyl)pyridin-2(1H)-one

To a solution of (Z)—N′-hydroxy-1-(3-(2-methoxyethoxy)benzyl)-6-oxo-1,6-dihydropyridine-3-carboximidamide (Example 65, Step 3; 500 mg, 1.57 mmol), 1-ethyl-3-(3-dimethylaminopropyl)carbodimide hydrochloride (450 mg, 2.36 mmol), N-hydroxybenzotrazole (320 mg, 2.36 mmol) and K₂CO₃ (440 mg, 3.1 mmol) in DMF (10 mL), was added 4-(4-methoxyphenylthio)benzoic acid (430 mg, 1.57 mmol). The mixture was stirred at 110° C. for 16 h, then cooled to RT, diluted with water (15 mL) and extracted with EtOAc (20 mL×3). The combined organic layers were washed with water (30 mL) and concentrated under reduced pressure. The residue was purified by SiO₂ gel chromatography (Hexanes:EtOAc=3:1) to give the title compound as a white solid (750 mg, 85.7%). MS(ES⁺) C₁₄H₁₂O₃S requires: 555. found: 556 [M+H]⁺.

### Step 2: 5-(5-(4-Mercaptophenyl)-1,2,4-oxadiazol-3-yl)-1-(3-(2-methoxyethoxy)benzyl)pyridin-2(1H)-one

To a solution of 5-(5-(4-mercaptophenyl)-1,2,4-oxadiazol-3-yl)-1-(3-(2-methoxyethoxy)benzyl)pyridin-2(1H)-one (750 mg, 1.35 mmol) in trifluoracetic acid (10 mL) was added triethoxysilane (443 mg, 2.7 mmol). The mixture was stirred at 80° C. for 16 h, then cooled to RT, diluted with water (15 mL) and extracted with EtOAc (15 mL×3). The combined organic layers were concentrated under reduced pressure and the residue was purified by SiO₂ gel chromatography (Hexanes:EtOAc=3:1) to afford the title compound as a white solid (480 mg, 81%). MS(ES⁺) C₂₃H₂₁N₃O₄S requires: 435. found: 436 [M+H]⁺.

### Step 3: 2-(4-(3-(1-(3-(2-Methoxyethoxy)benzyl)-6-oxo-1,6-dihydropyridin-3-yl)-1,2,4-oxadiazol-5-yl)phenylthio)acetonitrile

To a solution of 5-(5-(4-mercaptophenyl)-1,2,4-oxadiazol-3-yl)-1-(3-(2-methoxyethoxy)benzyl)pyridin-2(1H)-one (100 mg, 0.23 mmol) and K₂CO₃ (95 mg, 0.69 mmol) in DMF (8 mL) was added 2-chloroacetonitrile (87 mg, 1.15 mmol). The mixture was stirred at 55° C. for 16 h, then cooled to RT, diluted with water (10 mL) and extracted with EtOAc (10 mL×3). The combined organic layers were concentrated under reduced pressure and the residue was purified by prep-HPLC to afford the title compound as a white solid (80 mg, 73%). MS(ES⁺) C₂₅H₂₂N₄O₄S requires: 474. found: 475 [M+H]⁺.

### Step 4: 2-(4-(3-(1-(3-(2-Methoxyethoxy)benzyl)-6-oxo-1,6-dihydropyridin-3-yl)-1,2,4-oxadiazol-5-yl)phenylsulfonyl)acetonitrile

To a solution of 2-(4-(3-(1-(3-(2-methoxyethoxy)benzyl)-6-oxo-1,6-dihydropyridin-3-yl)-1,2,4-oxadiazol-5-yl)phenylthio)acetonitrile (60 mg, 0.13 mmol) in DCM (8 mL) was added 3-chlorobenzoperoxoic acid (67 mg, 0.39 mmol). The mixture was stirred at RT for 2 h, then concentrated under reduced pressure. The residue was purified by SiO₂ gel chromatography (Hexanes:EtOAc=3:1) to give the title compound as a white solid; MS(ES⁺) C₂₅H₂₂N₄O₆S requires: 506. found: 507 [M+H]⁺. ¹H NMR (500 MHz, CDCl₃) δ 8.46 (d, J=8.5 Hz, 2H), 8.24 (m, 3H), 8.01 (dd, J=9.4, 2.3 Hz, 1H), 7.29 (m, 1H), 7.02-6.92 (m, 2H), 6.91-6.86 (m, 1H), 6.74 (d, J=9.5 Hz, 1H), 5.22 (s, 2H), 4.14 (s, 2H), 4.13-4.09 (m, 2H), 3.76-3.72 (m, 2H), 3.44 (s, 3H).

### EXAMPLE 86

### 1-(3-(2-Methoxyethoxy)benzyl)-5-(5-(2-(methylthio)pyrimidin-5-yl)-1,2,4-oxadiazol-3-yl)pyridin-2(1H)-one

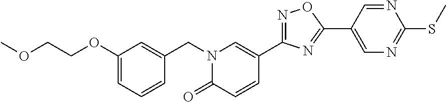

### Step 1

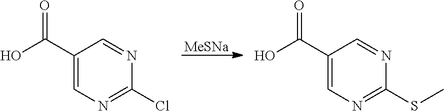

### 2-(Methylthio)pyrimidine-5-carboxylic acid

A mixture of 2-chloropyrimidine-5-carboxylic acid (50 mg, 0.31 mmol), sodium methanethiolate (43 mg, 0.62 mmol) and K₂CO₃ (85 mg, 0.62 mmol) in MeOH (10 mL) was stirred at RT for 16 h. The mixture was purified directly by prep-HPLC (Mobile phase: A=10 mM ammonium bicarbonate/H₂O, B=MeCN; Gradient: B=60%-95% in 18 min; Column: XBridge C18, 5 um, 30 mm×150 mm) to afford 2-(methylthio)pyrimidine-5-carboxylic acid as a white solid (40 mg, 74%). MS(ES⁺) C₆H₇N₃O₂ requires: 170. found: 171 [M+H]⁺.

### Step 2

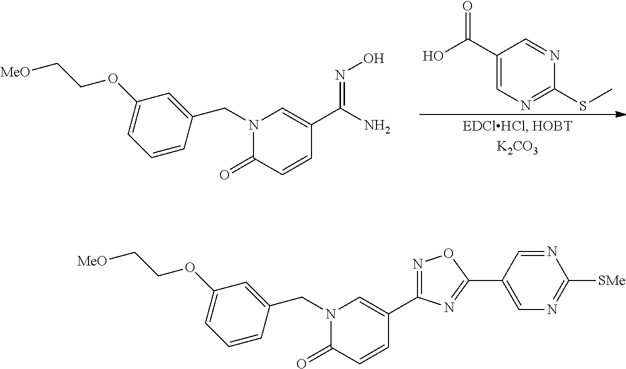 [SMILES: COCCOc1cccc(Cn2cc(/C(N)=N/O)ccc2=O)c1 ; CSc1ncc(C(=O)O)cn1 ; COCCOc1cccc(Cn2cc(-c3noc(-c4cnc(SC)nc4)n3)ccc2=O)c1]

### 1-(3-(2-Methoxyethoxy)benzyl)-5-(5-(2-(methylthio)pyrimidin-5-yl)-1,2,4-oxadiazol-3-yl)pyridin-2(1H)-one

To a solution of 2-(methylthio)pyrimidine-5-carboxylic acid (40 mg, 0.24 mmol) in DMF (5 mL) were added (Z)—N′-hydroxy-1-(3-(2-methoxyethoxy)benzyl)-6-oxo-1,6-dihydropyridine-3-carboximidamide (Example 65, Step 3; 124 mg, 0.39 mmol), 1-ethyl-3-(3-dimethylaminopropyl)carbodiimide hydrochloride (75 mg, 0.39 mmol), 1-hydroxybenzotriazole hydrate (22 mg, 0.13 mmol) and K₂CO₃ (72 mg, 0.52 mmol). The mixture was stirred at RT for 16 h, then diluted with H₂O (20 mL) and extracted with EtOAc (3×20 mL). The combined organic layers were washed with water (20 mL) and brine (20 mL), dried over Na₂SO₄, filtered and concentrated under reduced pressure. The residue was purified by prep-HPLC (Mobile phase: A=10 mM ammonium bicarbonate/H₂O, B=MeCN; Gradient: B=60% to 95% in 18 min; Column: XBridge C18, 5 um, 30 mm×150 mm) to afford the title compound as a white solid; MS(ES⁺) C₂₂H₂₁N₅O₄S requires: 451. found: 452 [M+H]⁺. ¹H NMR (500 MHz, CDCl₃) δ 9.18 (s, 2H), 8.23 (d, J=2.0 Hz, 1H), 8.00 (dd, J=9.5, 2.2 Hz, 1H), 7.29 (m, 1H), 6.98-6.89 (m, 3H), 6.74 (d, J=9.5 Hz, 1H), 5.21 (s, 2H), 4.17-4.02 (m, 2H), 3.84-3.68 (m, 2H), 3.45 (s, 3H), 2.65 (s, 3H).

### EXAMPLE 87

### 5-(5-(2-Isobutoxypyrimidin-5-yl)-1,2,4-oxadiazol-3-yl)-1-(3-(2-methoxyethoxy)benzyl)pyridin-2(1H)-one

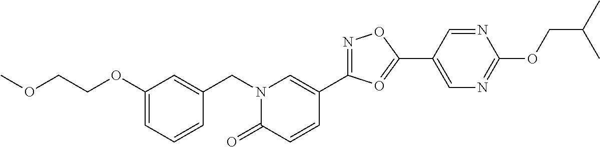

### Step 1

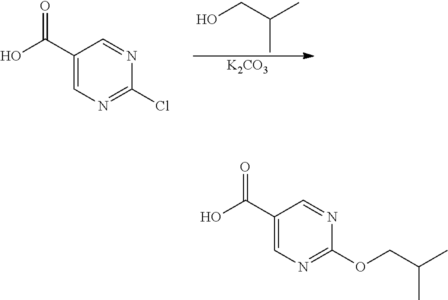

### 2-Isobutoxypyrimidine-5-carboxylic acid

A mixture of 2-chloropyrimidine-5-carboxylic acid (50 mg, 0.315 mmol), 2-methylpropan-1-ol (70 mg, 0.948 mmol) and K₂CO₃ (139 mg, 0.948 mmol) in EtOH (3 mL) was stirred at 30° C. for 16 h. The mixture was treated with 4M aq. hydrochloric acid (ca. 2 mL) until pH=4 was reached, and then purified directly by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to afford 2-isobutoxypyrimidine-5-carboxylic acid (71 mg, 97%). MS(ES⁺) C₉H₁₂N₂O₃ requires: 196. found: 197 [M+H]+.

### Step 2

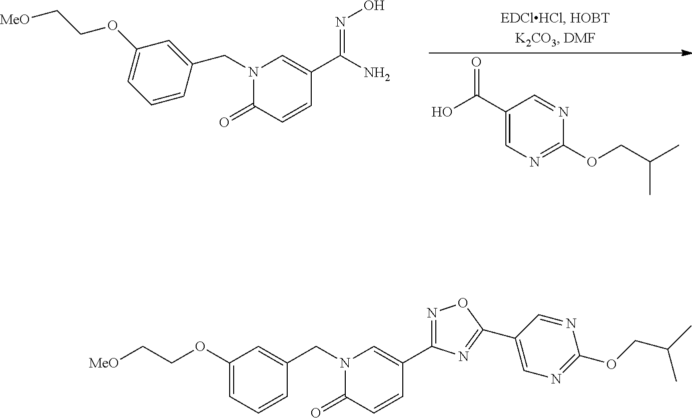 [SMILES: COCCOc1cccc(Cn2cc(/C(N)=N/O)ccc2=O)c1 ; CC(C)COc1ncc(C(=O)O)cn1 ; COCCOc1cccc(Cn2cc(-c3noc(-c4cnc(OCC(C)C)nc4)n3)ccc2=O)c1]

### 5-(5-(2-Isobutoxypyrimidin-5-yl)-1,2,4-oxadiazol-3-yl)-1-(3-(2-methoxyethoxy)benzyl)pyridin-2(1H)-one

To a solution of 2-(2-methoxyethoxy)pyrimidine-5-carboxylic acid (71 mg, 0.362 mmol) in DMF (2 mL) were added 3-propanediamine-N′-(ethylcarbonimidoyl)-N,N-dimethyl hydrochloride (104 mg, 0.543 mmol), 1-hydroxybenzotriazole (74 mg, 0.543 mmol), K₂CO₃ (100 mg, 0.724 mmol) and (Z)—N′-hydroxy-1-(3-(2-methoxyethoxy)benzyl)-6-oxo-1,6-dihydropyridine-3-carboximidamide (Example 65, Step 3; 115 mg, 0.362 mmol). The mixture was stirred at 50° C. for 16 h, then cooled to RT and filtered through a pad of Celite. The filtrate was purified by prep-HPLC (Mobile phase: A=0.1% ammonium hydroxide/H₂O, B=MeCN; Gradient: B=60% to 95% in 18 min; Column: XBridge (C18, Sum, 30 mm×150 mm) to afford the title compound; MS(ES⁺) C₂₅H₂₇N₅O₅ requires: 477. found: 478 [M+H]⁺; ¹H NMR (500 MHz, CDCl₃) δ 9.22 (s, 2H), 8.24 (d, J=1.9 Hz, 1H), 8.00 (dd, J=9.5, 2.1 Hz, 1H), 7.29 (m, 1H), 7.04-6.85 (m, 3H), 6.73 (d, J=9.5 Hz, 1H), 5.21 (s, 2H), 4.27 (d, J=6.7 Hz, 2H), 4.18-4.02 (m, 2H), 3.83-3.67 (m, 2H), 3.44 (s, 3H), 2.19 (m, 1H), 1.07 (d, J=6.7 Hz, 6H).

### EXAMPLE 88

### 1-(3-(2-Methoxyethoxy)benzyl)-5-(5-(2-(piperidin-1-yl)pyrimidin-5-yl)-1,2,4-oxadiazol-3-yl)pyridin-2(1H)-one

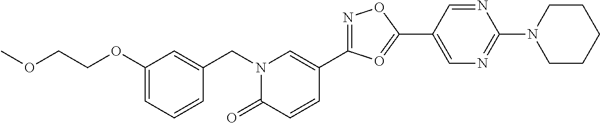

Synthetized in analogous manner to Example 87 using 2-(piperidin-1-yl)pyrimidine-5-carboxylic acid instead of 2-(2-methoxyethoxy)pyrimidine-5-carboxylic acid; MS(ES⁺) C₂₆H₂₈N₆O₄ requires: 488. found: 489 [M+H]⁺. ¹H NMR (500 MHz, CDCl₃) δ 8.95 (s, 2H), 8.22 (bs, 1H), 8.00 (m, 1H), 7.29 (m, 1H), 7.00-6.91 (m, 3H), 6.88 (bd, J=7.5 Hz, 1H), 5.21 (s, 2H), 4.16-4.05 (m, 2H), 3.94 (s, 4H), 3.79-3.68 (m, 2H), 3.42 (s, 3H), 1.70 (m, 6H).

### EXAMPLE 89

### 3-(4-(Tert-butyl)phenyl)-5-(5-methyl-4-(3-(4-methylpiperazin-1-yl)benzyl)pyrimidin-2-yl)-1,2,4-oxadiazole

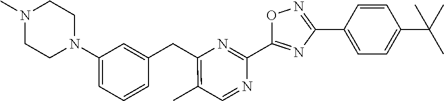 [SMILES: Cc1cnc(-c2nc(-c3ccc(C(C)(C)C)cc3)no2)nc1Cc1cccc(N2CCN(C)CC2)c1]

Synthesized from (Z)-4-(tert-butyl)-N′-hydroxybenzimidamide in an analogous manner to Example 19. MS(ES⁺) C₂₉H₃₄N₆O requires: 482. found: 483 [M+H]⁺; ¹H-NMR (600 MHz, CDCl₃) δ ppm 8.70 (s, 1H), 8.18 (d, J=7.2 Hz, 2H), 7.54 (d, J=7.2 Hz, 2H), 7.22 (m, 1H), 6.93 (s, 1H), 6.84 (dd, J=7.8, 2.4 Hz, 1H), 6.78 (d, J=7.8 Hz, 1H), 4.27 (s, 2H), 3.65 (m, 4H), 3.33 (m, 2H), 2.97 (m, 2H), 2.84 (s, 3H), 2.37 (s, 3H), 1.37 (s, 9H).

### EXAMPLE 90

### 5-(5-Methyl-6-(3-(4-methylpiperazin-1-yl)benzyl)pyrazin-2-yl)-3-(4-(trifluoromethoxy)phenyl)-1,2,4-oxadiazole

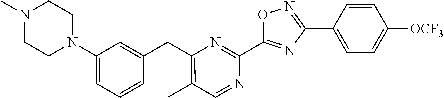 [SMILES: Cc1cnc(-c2nc(-c3ccc(OC(F)(F)F)cc3)no2)nc1Cc1cccc(N2CCN(C)CC2)c1]

### Steps 1 to 4:

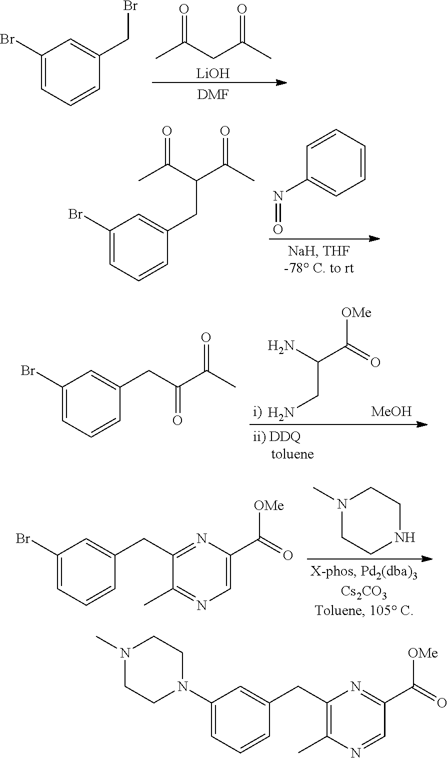

### Step 1: 3-(3-Bromobenzyl)pentane-2,4-dione

A mixture of 1-bromo-3-(bromomethyl)benzene (2.087 g, 8.35 mmol), pentane-2,4-dione (4.18 g, 41.8 mmol), and lithium hydroxide (0.5 g, 20.88 mmol) in DMF (30 mL) was heated to 75° C. for 30 minutes. The mixture was diluted with EtOAc and washed with brine. The organic layer was concentrated under reduced pressure and purified by SiO₂ gel chromatography (0% to 20% EtOAc in Hexanes) to give the title product as a clear oil (1.93 g, 86%). MS(ES⁺) C₁₂H₁₃BrO₂ requires: 268, 270 [M+2] found: 269 [M+H]⁺, 271 [M+2+H]⁺.

### Step 2: 1-(3-Bromophenyl)butane-2,3-dione

To a suspension of sodium hydride (60% dispersion in mineral oil, 149 mg, 3.72 mmol) in THF (8 mL) was added a solution of 3-(3-bromobenzyl)pentane-2,4-dione (1000 mg, 3.72 mmol) in THF (8 mL). The mixture was stirred for 20 minutes and was then cooled down to −78° C. A solution of nitrosobenzene (418 mg, 3.90 mmol) in THF (8 mL) was added and the reaction was stirred at RT for 20 minutes. 1N aq. HCl (24 mL) was added and the resulting mixture was then heated up to 50° C. for 40 minutes. The mixture was diluted with EtOAc and Hexanes (1:1 v:v, 30 mL) and washed with water. The organic layer was concentrated under reduced pressure. The residue was purified by SiO₂ gel chromatography (0% to 100% EtOAc in Hexanes) to give the title compound (596 mg, 67%). MS(ES⁺) C₁₀H₉BrO₂ requires: 240, 242 [M+2] found: 241 [M+H]⁺, 243 [M+2+H]⁺.

### Step 3: Methyl 6-(3-bromobenzyl)-5-methylpyrazine-2-carboxylate

A mixture of 1-(3-bromophenyl)butane-2,3-dione (500 mg, 2.074 mmol), methyl 2,3-diaminopropanoate hydrobromide salt (413 mg, 2.074 mmol), and triethylamine (0.862 ml, 6.22 mmol) in MeOH (5 mL) was heated to 65° C. for 2 h. MeOH was removed under reduced pressure and toluene (5.00 ml) was added, followed by DDQ (471 mg, 2.074 mmol). The mixture was stirred at RT for 1 h and then diluted with DCM. The solvent was then removed and the crude was purified by SiO₂ gel chromatography (0% to 100% EtOAc in Hexanes) to give the title compound (165 mg, 25%). MS(ES⁺) C₁₄H₁₃BrN₂O₂ requires: 320, 322 [M+2] found: 321 [M+H]⁺, 323 [M+2+H]⁺.

### Step 4: Methyl 5-methyl-6-(3-(4-methylpiperazin-1-yl)benzyl)pyrazine-2-carboxylate

A mixture of X-phos (44.5 mg, 0.093 mmol), tris(dibenzylideneacetone)dipalladium(0) (42.8 mg, 0.047 mmol), Cs₂CO₃ (203 mg, 0.623 mmol), methyl 6-(3-bromobenzyl)-5-methylpyrazine-2-carboxylate (100 mg, 0.311 mmol) and 1-methylpiperazine (0.069 ml, 0.623 mmol) in toluene (1.5 ml) was degassed with nitrogen. The mixture was stirred at 105° C. for 2 h, diluted with DCM and washed with water. The organic layer was concentrated under reduced pressure and purified by SiO₂ gel chromatography to give the title compound (64 mg, 60%). MS (ES⁺) C₁₉H₂₄N₄O₂ requires: 340 found: 341 [M+H]⁺.

### Step 5

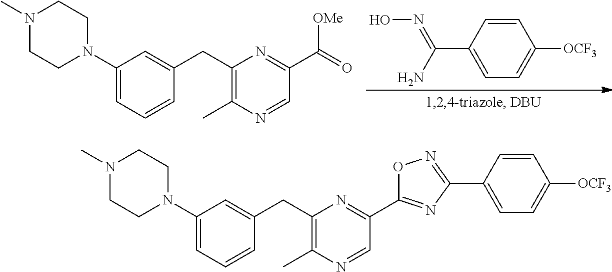

### 1-Methyl-4-(3-((3-methyl-6-(3-(4-(trifluoromethoxy)phenyl)-1,2,4-oxadiazol-5-yl)pyrazin-2-yl)methyl)phenyl)piperazin-1-ium 2,2,2-trifluoroacetate

A mixture of DBU (4.02 mg, 0.026 mmol), 1H-1,2,4-triazole (1.826 mg, 0.026 mmol), methyl 5-methyl-6-(3-(4-methylpiperazin-1-yl)benzyl)pyrazine-2-carboxylate (30.0 mg, 0.088 mmol) and (Z)—N′-hydroxy-4-(trifluoromethoxy)benzimidamide (38.8 mg, 0.176 mmol) in DMSO (0.1 ml) was stirred at 140° C. for 1 h. The mixture was then concentrated under reduced pressure and purified by prep HPLC to give the title compound; MS(ES⁺) C₂₆H₂₅F₃N₆O₂ requires: 510. found: 511 [M+H]⁺; ¹H-NMR (600 MHz, CD₃OD) δ ppm 9.25 (s, 1H), 8.30 (d, J=7.2 Hz, 2H), 7.50 (d, J=8.2 Hz, 2H), 7.24 (m, 1H), 6.99 (s, 1H), 6.91 (dd, J=7.8, 2.4 Hz, 1H), 6.83 (d, J=7.8 Hz, 1H), 4.36 (s, 2H), 3.82 (bd, J=13.2 Hz, 2H), 3.57 (bd, J=13.2 Hz, 2H), 3.23 (m, 2H), 3.01 (m, 2H), 2.94 (s, 3H), 2.62 (s, 3H).

### EXAMPLE 91

### 1-(3-(4-Methylpiperazin-1-yl)benzyl)-5-(5-(4-(trifluoromethoxy)phenyl)oxazol-2-yl)pyridin-2(1H)-one

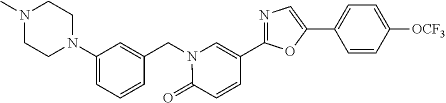 [SMILES: CN1CCN(c2cccc(Cn3cc(-c4ncc(-c5ccc(OC(F)(F)F)cc5)o4)ccc3=O)c2)CC1]

### Step 1

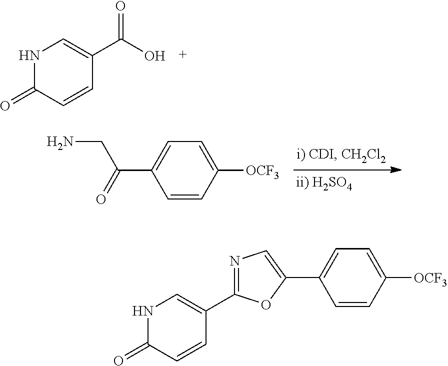

### 5-(5-(4-(Trifluoromethoxy)phenyl)oxazol-2-yl)pyridin-2(1H)-one

A mixture of di(1H-imidazol-1-yl)methanone (127 mg, 0.784 mmol), 6-oxo-1,6-dihydropyridine-3-carboxylic acid (100 mg, 0.719 mmol) and 2-amino-1-(4-(trifluoromethoxy)phenyl)ethanone (143 mg, 0.654 mmol) in DCM (3 ml) was stirred at RT for 8 h. The resulting suspension was filtered and the solid product was dissolved in concentrated sulfuric acid (0.2 mL). The mixture was stirred at RT for 30 minutes and then poured into cold water. The resulting suspension was filtered to give the title compound as a solid which was used in the next step without further purification (169 mg, 80%). MS(ES⁺) C₁₅H₉F₃N₂O₃ requires: 322 found: 323 [M+H]⁺.

### Step 2

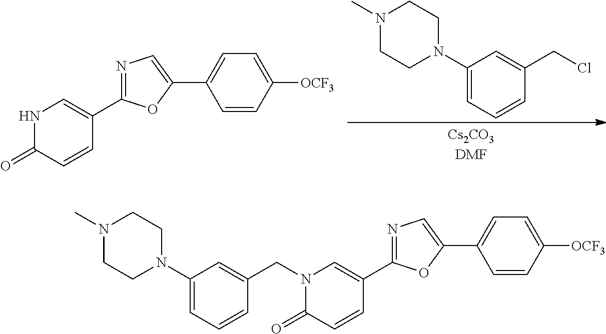 [SMILES: O=c1ccc(-c2ncc(-c3ccc(OC(F)(F)F)cc3)o2)c[nH]1 ; CN1CCN(c2cccc(CCl)c2)CC1 ; CN1CCN(c2cccc(Cn3cc(-c4ncc(-c5ccc(OC(F)(F)F)cc5)o4)ccc3=O)c2)CC1]

### 1-Methyl-4-(3-((2-oxo-5-(5-(4-(trifluoromethoxy)phenyl)oxazol-2-yl)pyridin-1(2H)-yl)methyl)phenyl)piperazin-1-ium 2,2,2-trifluoroacetate

Synthetized from 5-(5-(4-(trifluoromethoxy)phenyl)oxazol-2-yl)pyridin-2(1H)-one (23.01 mg, 0.0714 mmol) and 1-(3-(chloromethyl)phenyl)-4-methylpiperazine

(Example 100, Step 2) as described for Example 26; MS(ES⁺) C₂₇H₂₅F₃N₄O₃ requires: 510. found: 511 [M+H]⁺; ¹H-NMR (600 MHz, CD₃OD) δ ppm 8.57 (d, J=2.4 Hz, 1H), 8.16 (dd, J=2.3, 9.5 Hz, 1H), 7.88 (d, J=9.0 Hz, 2H), 7.61 (s, 1H), 7.38 (d, J=8.5 Hz, 2H), 7.30 (m, 1H), 7.08 (s, 1H), 7.00 (dd, J=2.3, 8.5 Hz, 1H), 6.97 (d, J=7.3 Hz, 1H), 6.71 (d, J=8.3 Hz, 1H), 5.27 (s, 2H), 3.86 (bd, J=13.2 Hz, 2H), 3.58 (bd, J=13.2 Hz, 2H), 3.25 (m, 2H), 3.03 (m, 2H), 2.95 (s, 3H).

### EXAMPLE 92

### 1-((2-(4-Methylpiperazin-1-yl)pyridin-4-yl)methyl)-5-(5-(4-(trifluoromethoxy)phenyl)oxazol-2-yl)pyridin-2(1H)-one

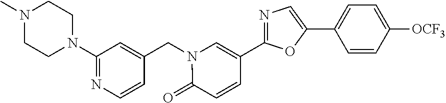 [SMILES: CN1CCN(c2cc(Cn3cc(-c4ncc(-c5ccc(OC(F)(F)F)cc5)o4)ccc3=O)ccn2)CC1]

Synthetized from 5-(5-(4-(trifluoromethoxy)phenyl)oxazol-2-yl)pyridin-2(1H)-one (Example 91, Step 1) in analogous manner to Example 7; MS(ES⁺) C₂₆H₂₄F₃N₅O₃ requires: 511. found: 512 [M+H]⁺; ¹H-NMR (600 MHz, CD₃OD) δ ppm 8.64 (d, J=2.4 Hz, 1H), 8.19 (dd, J=2.5, 9.6 Hz, 1H), 8.12 (d, J=5.4 Hz, 1H), 7.88 (d, J=9.0 Hz, 2H), 7.63 (s, 1H), 7.36 (d, J=8.5 Hz, 2H), 7.11 (s, 1H), 6.84 (d, J=5.2 Hz, 1H), 6.72 (d, J=9.5 Hz, 1H), 5.32 (s, 2H), 3.11-4.15 (b, 8H), 2.96 (s, 3H).

### EXAMPLE 93

### 1-(3-(4-Methylpiperazin-1-yl)benzyl)-5-(4-(4-(trifluoromethoxy)phenyl)oxazol-2-yl)pyridin-2(1H)-one

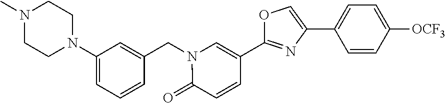 [SMILES: CN1CCN(c2cccc(Cn3cc(-c4nc(-c5ccc(OC(F)(F)F)cc5)co4)ccc3=O)c2)CC1]

### Steps 1 to 4

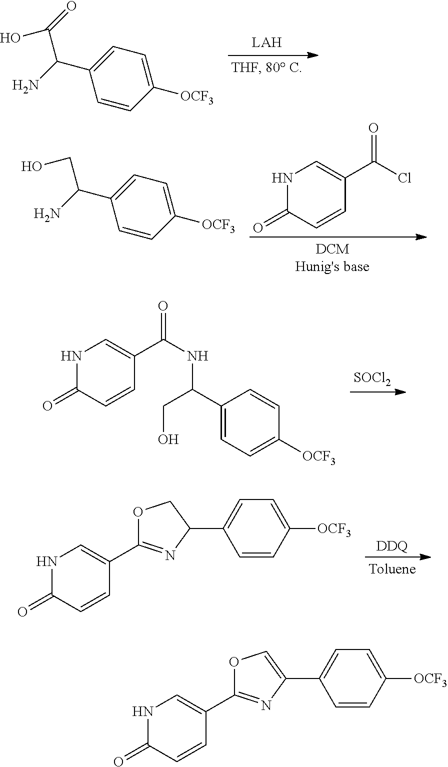

### Step 1: 2-Amino-2-(4-(trifluoromethoxy)phenyl)ethanol

To a solution of 2-amino-2-(4-(trifluoromethoxy)phenyl)acetic acid (200 mg, 0.850 mmol) in THF (1 mL) was added dropwise a solution of lithium aluminum hydride (1 M solution in THF, 4.25 mL, 4.25 mmol). The mixture was stirred at 80° C. for 3 h, cooled to RT and then quenched with water (0.05 mL) followed by NaOH (50 mg). The mixture was then diluted with DCM (30 mL) and heated up to 45° C. for 30 minutes. The mixture was filtered to remove the white precipitate and the filtrate was concentrated under reduced pressure to afford the title compound as a solid, which was used in the next step without further purification. MS(ES⁺) C₉H₁₀F₃NO₂ requires: 221 found: 222 [M+H]⁺.

### Step 2: N-(2-Hydroxy-1-(4-(trifluoromethoxy)phenyl)ethyl)-6-oxo-1,6-dihydropyridine-3-carboxamide

A mixture of 4-dimethylaminopyridine (2.209 mg, 0.018 mmol), N,N-diisopropylethylamine (0.126 mL, 0.723 mmol), 2-amino-2-(4-(trifluoromethoxy)phenyl)ethanol (80 mg, 0.362 mmol), and 6-oxo-1,6-dihydropyridine-3-carbonyl chloride (57.0 mg, 0.362 mmol) in DCM was stirred at RT for 2 h. MeOH (2 mL) was added followed by NaOH (200 mg) and the mixture was stirred at RT for further 10 minutes. The reaction mixture was concentrated under reduced pressure and the residue was purified by SiO₂ gel chromatography (0% to 40% MeOH in EtOAc) to give the title compound (60 mg, 49%). MS(ES⁺) C₁₅H₁₃F₃N₂O₄ requires: 342 found: 343 [M+H]⁺.

### Step 3: 5-(4-(4-(Trifluoromethoxy)phenyl)-4,5-dihydrooxazol-2-yl)pyridin-2(1H)-one

A mixture of N-(2-hydroxy-1-(4-(trifluoromethoxy)phenyl)ethyl)-6-oxo-1,6-dihydropyridine-3-carboxamide (60 mg, 0.175 mmol) in thionyl chloride (209 mg, 1.753 mmol) was stirred at RT for 2 h. The mixture was then concentrated under reduced pressure, the residue was dissolved in EtOAc and washed with water. The organic layer was dried over Na₂SO₄, filtered and concentrated under reduced pressure to give the title compound (20 mg, 35%), which was used in the next step without further purification. MS(ES⁺) C₁₅H₁₁F₃N₂O₃ requires: 324 found: 325 [M+H]⁺.

### Step 4: 5-(4-(4-(Trifluoromethoxy)phenyl)oxazol-2-yl)pyridin-2(1H)-one

A mixture of DDQ (14.00 mg, 0.062 mmol) and 5-(4-(4-(trifluoromethoxy)phenyl)-4,5-dihydrooxazol-2-yl)pyridin-2(1H)-one (20 mg, 0.062 mmol) in toluene (1 mL) was stirred at 100° C. for 30 minutes. The mixture was then directly purified by SiO₂ gel chromatography (0% to 100% EtOAc in Hexanes and then 0% to 40% MeOH in EtOAc) to give the title compound (10 mg, 50%). MS(ES⁺) C₁₅H₉F₃N₂O₃ requires: 322 found: 323 [M+H]⁺.

### Step 5

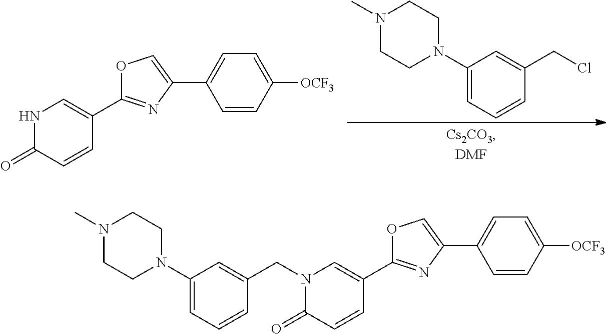 [SMILES: O=c1ccc(-c2nc(-c3ccc(OC(F)(F)F)cc3)co2)c[nH]1 ; CN1CCN(c2cccc(CCl)c2)CC1 ; CN1CCN(c2cccc(Cn3cc(-c4nc(-c5ccc(OC(F)(F)F)cc5)co4)ccc3=O)c2)CC1]

### 1-Methyl-4-(3-((2-oxo-5-(4-(4-(trifluoromethoxy)phenyl)oxazol-2-yl)pyridin-1(2H)-yl)methyl)phenyl)piperazin-1-ium 2,2,2-trifluoroacetate

Synthetized in an analogous manner to Example 26 using 5-(4-(4-(trifluoromethoxy)phenyl)oxazol-2-yl)pyridin-2(1H)-one (6 mg, 0.019 mmol) and 1-(3-(chloromethyl)phenyl)-4-methylpiperazine (Example 100, Step 2; 8.37 mg, 0.037 mmol; MS(ES⁺) C₂₇H₂₅F₃N₄O₃ requires: 510. found: 511 [M+H]⁺; ¹H-NMR (600 MHz, CDCl₃) δ ppm 8.16 (d, J=2.6 Hz, 1H), 8.03 (dd, J=2.3, 9.6 Hz, 1H), 7.88 (s, 1H), 7.77 (d, J=9.0 Hz, 2H), 7.31 (m, 1H), 7.27 (d, J=9.5 Hz, 2H), 6.98 (s, 1H), 6.95 (d, J=7.2 Hz, 1H), 6.88 (d, J=2.3, 9.0 Hz, 1H), 6.73 (d, J=9.3 Hz, 1H), 5.18 (s, 2H), 3.68 (m, 4H), 3.31 (m, 2H), 2.95 (m, 2H), 2.85 (s, 3H).

### EXAMPLE 94

### 1-(3-(2-Methoxyethoxy)benzyl)-5-(4-(4-(trifluoromethoxy)phenyl)oxazol-2-yl)pyridin-2(1H)-one

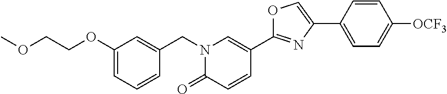 [SMILES: COCCOc1cccc(Cn2cc(-c3nc(-c4ccc(OC(F)(F)F)cc4)co3)ccc2=O)c1]

Synthesized from 5-(4-(4-(trifluoromethoxy)phenyl)oxazol-2-yl)pyridin-2(1H)-one (Example 93, Step 4) in an analogous method to Example 33. MS(ES⁺) C₂₅H₂₁F₃N₂O₅: 486. found: 487 [M+H]⁺; ¹H-NMR (600 MHz, DMSO-d₆) δ ppm 8.72 (s, 1H), 8.64 (d, J=2.4 Hz, 1H), 8.02 (dd, J=2.3, 9.5 Hz, 1H), 7.95 (d, J=9.1 Hz, 2H), 7.45 (d, J=9.1 Hz, 2H), 7.26 (m, 1H), 6.93 (m, 2H), 6.87 (d, J=7.3 Hz, 1H), 6.62 (d, J=9.3 Hz, 1H), 5.20 (s, 2H), 4.07 (m, 2H), 3.65 (m, 2H), 3.29 (s, 3H).

### EXAMPLE 95

### 1-(3-(3-(Methylsulfonyl)propoxy)benzyl)-5-(4-(4-(trifluoromethoxy)phenyl)oxazol-2-yl)pyridin-2(1H)-one

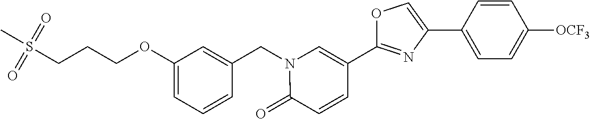 [SMILES: CS(=O)(=O)CCCOc1cccc(Cn2cc(-c3nc(-c4ccc(OC(F)(F)F)cc4)co3)ccc2=O)c1]

Synthesized from 5-(4-(4-(trifluoromethoxy)phenyl)oxazol-2-yl)pyridin-2(1H)-one (Example 93, Step 4) in an analogous manner to Example 33. MS(ES⁺) C₂₆H₂₃F₃N₂O₆S requires: 548. found: 549 [M+H]⁺; ¹H NMR (600 MHz, DMSO-d₆) δ ppm 8.72 (s, 1H), 8.64 (d, J=2.4 Hz, 1H), 8.02 (dd, J=2.3, 9.5 Hz, 1H), 7.95 (d, J=9.1 Hz, 2H), 7.47 (d, J=9.1 Hz, 2H), 7.28 (m, 1H), 6.94 (m, 2H), 6.88 (d, J=7.3 Hz, 1H), 6.62 (d, J=9.3 Hz, 1H), 5.22 (s, 2H), 4.07 (t, J=13.2 Hz, 2H), 3.36 (t, J=13.2 Hz, 2H), 3.00 (s, 3H), 2.12 (m, 2H).

### EXAMPLE 96

### 1-(3-(4-Methylpiperazin-1-yl)benzyl)-5-(5-(4-(trifluoromethoxy)phenyl)isoxazol-3-yl)pyridin-2(1H)-one (Example 96a) and 1-(3-(4-methylpiperazin-1-yl)benzyl)-5-(3-(4-(trifluoromethoxy)phenyl)isoxazol-5-yl)pyridin-2(1H)-on Example 96b)

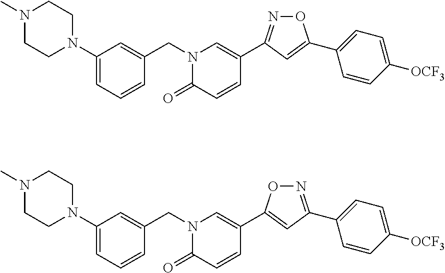 [SMILES: CN1CCN(c2cccc(Cn3cc(-c4cc(-c5ccc(OC(F)(F)F)cc5)on4)ccc3=O)c2)CC1 ; CN1CCN(c2cccc(Cn3cc(-c4cc(-c5ccc(OC(F)(F)F)cc5)no4)ccc3=O)c2)CC1]

### Steps 1 to 3

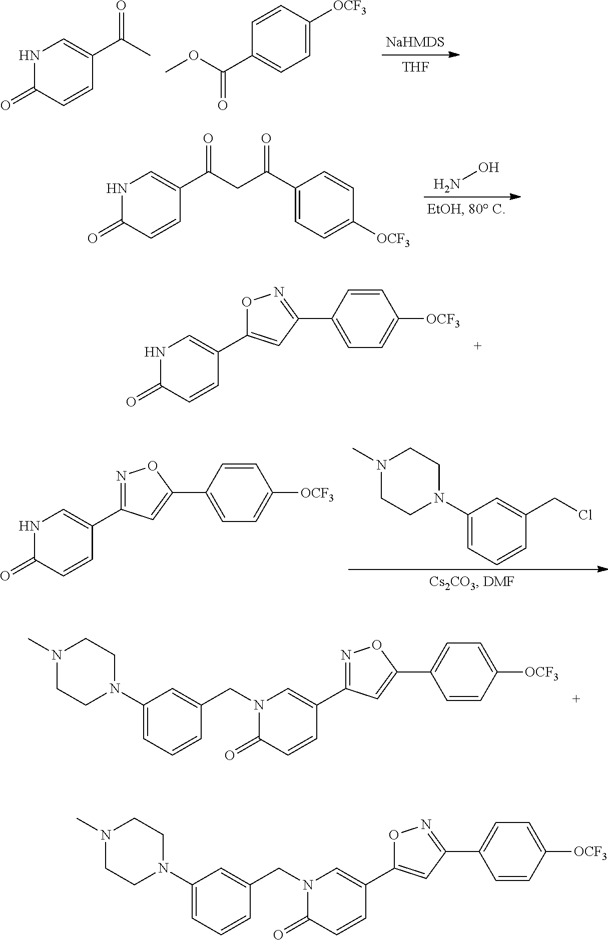

### Step 1: 1-(6-oxo-1,6-dihydropyridin-3-yl)-3-(4-(trifluoromethoxy)phenyl)propane-1,3-dione

To a solution of 5-acetylpyridin-2(1H)-one (50 mg, 0.365 mmol) in THF (2 mL) at −78° C. was added dropwise NaHMDS (0.419 mL, 0.839 mmol). The mixture was warmed up to RT and stirred for 10 minutes. Methyl 4-(trifluoromethoxy)benzoate (225 mg, 1.021 mmol) was subsequently added, the mixture was warmed up to 65° C. and stirred for 1.5 h. The reaction was quenched by puring into a 1M aq. HCl solution. The mixture was diluted with EtOAc and Hexanes, and let sit overnight and RT, resulting in the precipitation of the title compound as a yellow solid which was collected by filtration (96 mg, 81%) and used in the next step without further purification. MS(ES⁺) C₁₅H₁₀F₃NO₄ requires: 325 found: 326 [M+H]⁺.

### Step 2: 5-(3-(4-(Trifluoromethoxy)phenyl)isoxazol-5-yl)pyridin-2(1H)-one and 5-(5-(4-(trifluoromethoxy)phenyl)isoxazol-3-yl)pyridin-2(1H)-one

A mixture of 1-(6-oxo-1,6-dihydropyridin-3-yl)-3-(4-(trifluoromethoxy)phenyl)propane-1,3-dione (30 mg, 0.092 mmol) and hydroxylamine (30.5 mg, 0.922 mmol) in EtOH (1.5 mL) was heated to 80° C. and stirred for 6 h. The mixture was then diluted with EtOAc and washed with water. The organic layer was dried over Na₂SO₄, filtered and concentrated under reduced pressure to give the crude product as a regioisomeric mixture (ca. 3:1). MS(ES⁺) C₁₅H₉F₃N₂O₃ requires: 322. found: 323 [M+H]⁺.

### Step 3: 1-Methyl-4-(3-((2-oxo-5-(3-(4-(trifluoromethoxy)phenyl)isoxazol-5-yl)pyridin-1(2H)-yl)methyl)phenyl)piperazin-1-ium 2,2,2-trifluoroacetate and 1-methyl-4-(3-((2-oxo-5-(5-(4-(trifluoromethoxy)phenyl)isoxazol-3-yl)pyridin-1(2H)-yl)methyl)phenyl)piperazin-1-ium 2,2,2-trifluoroacetate

A mixture of 5-(3-(4-(trifluoromethoxy)phenyl)isoxazol-5-yl)pyridin-2(1H)-one and 5-(5-(4-(trifluoromethoxy)phenyl)isoxazol-3-yl)pyridin-2(1H)-one (ca. 3:1, 30 mg, 0.094 mmol), Cs₂CO₃ (179 mg, 0.549 mmol), and 1-(3-(chloromethyl)phenyl)-4-methylpiperazine (Example 100, Step 2, 30.8 mg, 0.137 mmol) in DMF (1.0 ml) was stirred at RT for 16 h. The reaction mixture was then purified directly by prep-HPLC to give the two isomeric title compounds; 1-methyl-4-(3-((2-oxo-5-(5-(4-(trifluoromethoxy)phenyl)isoxazol-3-yl)pyridin-1(2H)-yl)methyl)phenyl)piperazin-1-ium 2,2,2-trifluoroacetate (Example 96a); MS(ES⁺) C₂₇H₂₅F₃N₄O₃ requires: 510. found: 511 [M+H]⁺; ¹H-NMR (600 MHz, CD₃OD) δ ppm 8.37 (d, J=2.4 Hz, 1H), 8.04 (dd, J=2.3, 9.5 Hz, 1H), 7.95 (d, J=8.3 Hz, 2H), 7.45 (d, J=8.5 Hz, 2H), 7.30 (t, J=8.0 Hz, 1H), 7.20 (s, 1H), 7.07 (s, 1H), 6.98 (dd, J=2.3, 8.5 Hz, 1H), 6.96 (d, J=7.3 Hz, 1H), 6.71 (d, J=9.4 Hz, 1H), 5.24 (s, 2H), 3.85 (bd, J=13.2 Hz, 2H), 3.58 (bd, J=13.2 Hz, 2H), 3.25 (bt, J=12.6 Hz, 2H), 3.02 (bt, J=12.6 Hz, 2H), 2.95 (s, 3H). 1-methyl-4-(3-((2-oxo-5-(3-(4-(trifluoromethoxy)phenyl)isoxazol-5-yl)pyridin-1(2H)-yl)methyl)phenyl)piperazin-1-ium 2,2,2-trifluoroacetate (Example 96b): MS(ES⁺) C₂₇H₂₅F₃N₄O₃ requires: 510. found: 511 [M+H]⁺; ¹H-NMR (600 MHz, CD₃OD) δ ppm 8.42 (d, J=2.6 Hz, 1H), 7.97 (m, 3H), 7.42 (d, J=8.5 Hz, 2H), 7.30 (m, 1H), 7.12 (s, 1H), 7.09 (s, 1H), 7.00 (dd, J=2.3, 8.5 Hz, 1H), 6.96 (d, J=7.3 Hz, 1H), 6.70 (d, J=9.4 Hz, 1H), 5.24 (s, 2H), 3.85 (bd, J=13.2 Hz, 2H), 3.60 (bd, J=13.2 Hz, 2H), 3.25 (bt, J=12.6 Hz, 2H), 3.04 (bt, J=12.6 Hz, 2H), 2.94 (s, 3H).

### EXAMPLE 97

### 1-((2-(4-Methylpiperazin-1-yl)pyridin-4-yl)methyl)-5-(5-(4-(trifluoromethoxy)phenyl)isoxazol-3-yl)pyridin-2(1H)-one (Example 97a) and 1-((2-(4-methyl piperazin-1-yl)pyridin-4-yl)methyl)-5-(3-(4-(trifluoromethoxy)phenyl)isoxazol-5-yl)pyridin-2(1H)-one (Example 97b)

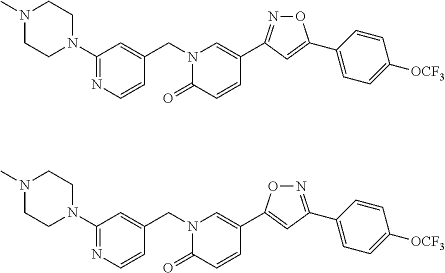 [SMILES: CN1CCN(c2cc(Cn3cc(-c4cc(-c5ccc(OC(F)(F)F)cc5)on4)ccc3=O)ccn2)CC1 ; CN1CCN(c2cc(Cn3cc(-c4cc(-c5ccc(OC(F)(F)F)cc5)no4)ccc3=O)ccn2)CC1]

### Steps 1 to 4

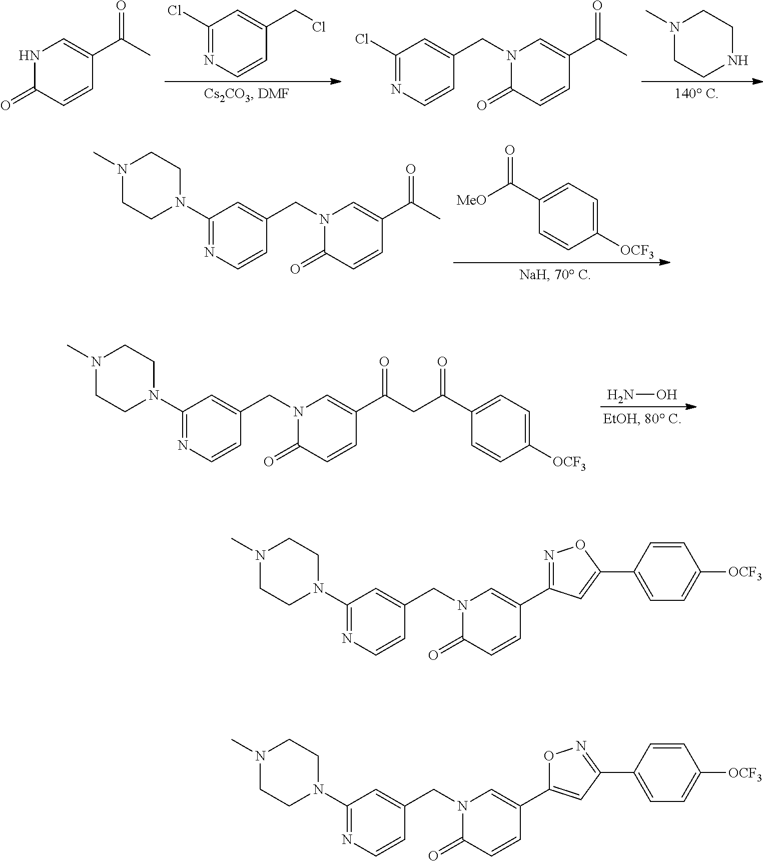

### Step 1: 5-Acetyl-1-((2-chloropyridin-4-yl)methyl)pyridin-2(1H)-one

A mixture of 5-acetylpyridin-2(1H)-one (300 mg, 2.188 mmol), Cs₂CO₃ (927 mg, 2.84 mmol), and 2-chloro-4-(chloromethyl)pyridine (461 mg, 2.84 mmol) in DMF (8 mL) was stirred at RT for 16 h. The mixture was then diluted with EtOAc and washed with water. The organic layer was dried over Na₂SO₄, filtered and concentrated under reduced pressure. The residue was purified by SiO₂ gel chromatography (20% to 100% EtOAc in Hexanes) to give the title compound as a solid (496 mg, 86%). MS(ES⁺) C₁₃H₁₁ClN₂O₂ requires: 262 found: 263 [M+H]⁺.

### Step 2: 5-Acetyl-1-((2-(4-methylpiperazin-1-yl)pyridin-4-yl)methyl)pyridin-2(1H)-one

A mixture of 5-acetyl-1-((2-chloropyridin-4-yl)methyl)pyridin-2(1H)-one (340 mg, 1.294 mmol) and 1-methylpiperazine (1945 mg, 19.41 mmol) was stirred at 140° C. for 6 h. The mixture was concentrated under reduced pressure and the residue was used in the next step without further purification. MS(ES⁺) C₁₈H₂₂N₄O₂ requires: 326. found: 327 [M+H]⁺.

### Step 3: 1-(1-((2-(4-Methylpiperazin-1-yl)pyridin-4-yl)methyl)-6-oxo-1,6-dihydropyridin-3-yl)-3-(4-(trifluoromethoxy)phenyl)propane-1,3-dione

A mixture of 5-acetyl-1-((2-(4-methylpiperazin-1-yl)pyridin-4-yl)methyl)pyridin-2(1H)-one (50.0 mg, 0.153 mmol), methyl 4-(trifluoromethoxy)benzoate (67.4 mg, 0.306 mmol), and sodium hydride (24.51 mg, 0.613 mmol) was stirred at 65° C. for 5 h. The mixture was then quenched with 1N aq. HCl. The mixture was concentrated under reduced pressure and the residue was purified by prep-HPLC to give the title compound (32 mg, 41%). MS(ES⁺) C₂₆H₂₅F₃N₄O₄ requires: 514. found: 515 [M+H]⁺.

### Step 4: 1-((2-(4-Methylpiperazin-1-yl)pyridin-4-yl)methyl)-5-(5-(4-(trifluoromethoxy)phenyl)isoxazol-3-yl)pyridin-2(1H)-one and 1-((2-(4-methyl piperazin-1-yl)pyridin-4-yl)methyl)-5-(3-(4-(trifluoromethoxy)phenyl)isoxazol-5-yl)pyridin-2(1H)-one

A mixture of 1-(1-((2-(4-methylpiperazin-1-yl)pyridin-4-yl)methyl)-6-oxo-1,6-dihydropyridin-3-yl)-3-(4-(trifluoromethoxy)phenyl)propane-1,3-dione (20 mg, 0.039 mmol) and hydroxylamine (3.85 mg, 0.117 mmol) in EtOH (0.5 mL) was stirred at 80° C. for 12 h. The volatiles were removed under reduced pressure and the mixture of oxazole isomers was purified by prep-HPLC; 1-((2-(4-methylpiperazin-1-yl)pyridin-4-yl)methyl)-5-(5-(4-(trifluoromethoxy)phenyl)isoxazol-3-yl)pyridin-2(1H)-one (Example 97a); MS(ES⁺) C₂₆H₂₄F₃N₅O₃ requires: 511. found: 512 [M+H]⁺; ¹H-NMR (600 MHz, CD₃OD) δ ppm 8.41 (d, J=2.4 Hz, 1H), 8.14 (d, J=5.4 Hz, 1H), 8.08 (dd, J=2.3, 9.5 Hz, 1H), 7.97 (d, J=8.5 Hz, 2H), 7.46 (d, J=8.5 Hz, 2H), 7.21 (s, 1H), 6.89 (s, 1H), 6.73 (d, J=9.0 Hz, 2H), 5.24 (s, 2H), 4.46 (m, 2H), 3.55 (m, 2H), 3.18 (m, 4H), 2.95 (s, 3H); 1-((2-(4-methyl piperazin-1-yl)pyridin-4-yl)methyl)-5-(3-(4-(trifluoromethoxy)phenyl)isoxazol-5-yl)pyridin-2(1H)-one (Example 97b); MS(ES⁺) C₂₆H₂₄F₃N₅O₃ requires: 511. found: 512 [M+H]⁺; H-NMR (600 MHz, CD₃OD) δ ppm 8.47 (d, J=2.4 Hz, 1H), 8.14 (d, J=5.4 Hz, 1H), 8.04 (dd, J=2.3, 9.5 Hz, 1H), 7.98 (d, J=8.5 Hz, 2H), 7.43 (d, J=8.5 Hz, 2H), 7.15 (s, 1H), 6.94 (s, 1H), 6.74 (s, 1H), 6.74 (d, J=9.0 Hz, 2H), 5.26 (s, 2H), 3.85-3.08 (m, 8H), 2.95 (s, 3H).

### EXAMPLE 98

### 1-(3-(4-(Methylsulfonyl)piperidin-1-yl)benzyl)-5-(3-(4-(trifluoromethoxy)phenyl)isoxazol-5-yl)pyridin-2(1H)-one

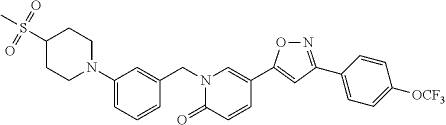 [SMILES: CS(=O)(=O)C1CCN(c2cccc(Cn3cc(-c4cc(-c5ccc(OC(F)(F)F)cc5)no4)ccc3=O)c2)CC1]

### Step 1

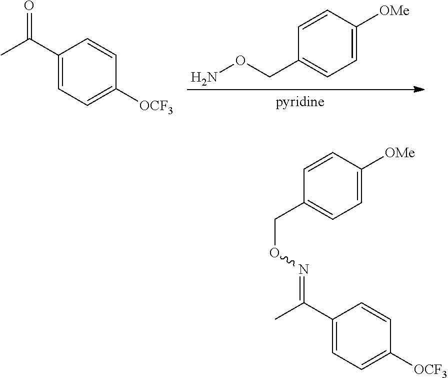 [SMILES: CC(=O)c1ccc(OC(F)(F)F)cc1 ; COc1ccc(CON)cc1 ; COc1ccc(CON=C(C)c2ccc(OC(F)(F)F)cc2)cc1]

### 1-(4-(Trifluoromethoxy)phenyl)ethan-1-one O-(4-(trifluoromethoxy)benzyl)oxime

To a solution of 1-(4-(trifluoromethoxy)phenyl)ethan-1-one (500 mg, 2.5 mmol) in pyridine (2.5 mL) was added O-(4-methoxybenzyl)hydroxylamine hydrochloride (488 mg, 2.5 mmol). The mixture was stirred at RT for 18 h. The reaction mixture was partitioned between CH₂Cl₂ (10 mL) and H₂O (10 mL). The organic layer was washed with H₂O (2×10 mL) and brine (2×10 mL), dried with Na₂SO₄, filtered, and concentrated under reduced pressure to give the title compound as a white solid (388 mg, 47%). MS(ES⁺) C₁₇H₁₆F₃NO₃ requires: 339. found: 340 [M+H]⁺.

### Step 2

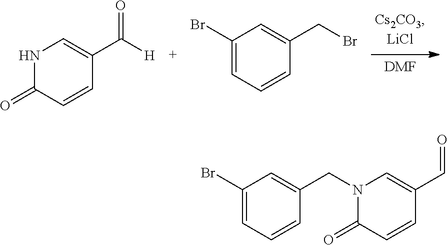

### 1-(3-Bromobenzyl)-6-oxo-1,6-dihydropyridine-3-carbaldehyde

To a mixture of 6-oxo-1,6-dihydropyridine-3-carbaldehyde (500 mg, 4.0 mmol), 1-bromo-3-(bromomethyl)benzene (2.0 g, 8.1 mmol) and LiCl (344 mg, 8.1 mmol) in DMF (20 mL) was added Cs₂CO₃ (1.9 g, 6.0 mmol) at RT. The mixture was then heated to 65° C. for 18 h. Upon cooling the mixture was pardoned between EtOAc (50 mL) and H₂O (50 mL). The organic layer was washed with H₂O (3×50 mL), brine (50 mL), dried over Na₂SO₄, filtered and concentrated under reduced pressure. The residue was purified by SiO₂ gel chromatography with (20% to 70% EtOAc in Hexanes) to give the title compound as an off-white solid (3.3 g, 83%). MS(ES⁺) C₁₃H₁₀BrNO₂ requires: 292. found: 294 [M+H]⁺.

### Steps 3 to 6

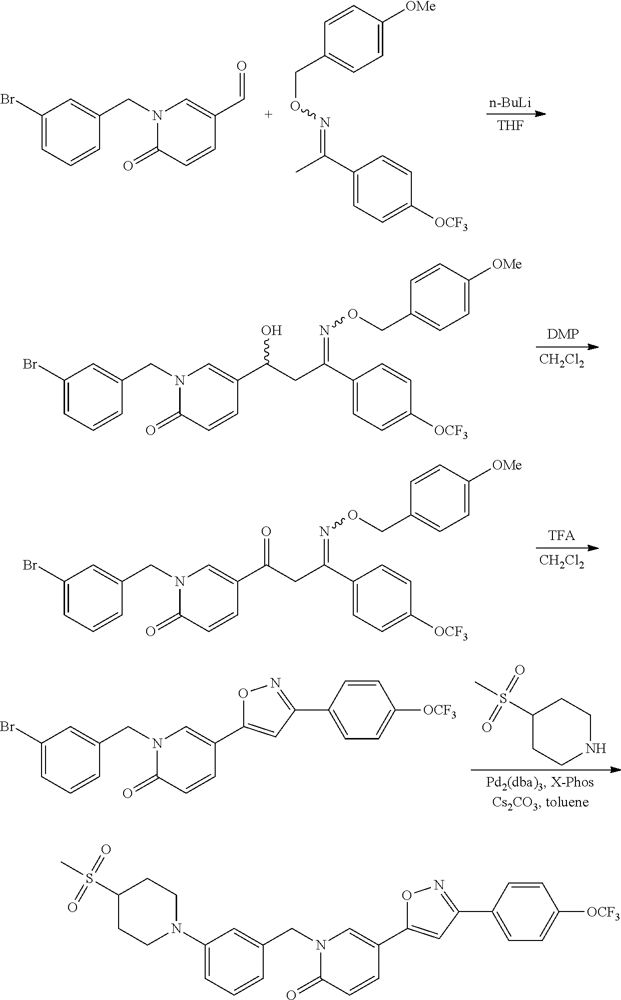

### Step 3: 1-(3-Bromobenzyl)-5-(1-hydroxy-3-(((4-methoxybenzyl)oxy)imino)-3-(4-(trifluoromethoxy)phenyl)propyl)pyridin-2(1H)-one

To a solution of 1-(4-(trifluoromethoxy)phenyl)ethan-1-one O-(4-methoxybenzyl)oxime (100 mg, 0.29 mmol) in THF (3.0 mL) at −78° C. was added dropwise n-BuLi (2.5 M in Hexane, 0.18 mL, 0.44 mmol), and the resulting mixture was stirred at −78° C. for 1 h. A solution of 1-(3-bromobenzyl)-6-oxo-1,6-dihydropyridine-3-carbaldehyde (95 mg, 0.32 mmol) in THF (0.5 mL) was added dropwise to the reaction mixture at −78° C. and the mixture was further stirred at this temperature for 6 h. NH₄Cl (aq. sat., 3 mL) was added and the mixture was allowed to warm to RT, diluted with EtOAc (15 mL) and washed with H₂O (2×15 mL). The organic layer was washed with brine (2×15 mL), dried with Na₂SO₄, filtered and concentrated under reduced pressure. The residue was purified by SiO₂ gel chromatography with (0% to 80% EtOAc in Hexanes) to give the title compound as a yellow oil (112 mg, 60%). MS(ES⁺) C₃₀H₂₆BrF₃N₂O₅ requires: 631. found: 633[M+H]⁺.

### Step 4: 1-(3-Bromobenzyl)-5-(3-(((4-methoxybenzyl)oxy)imino)-3-(4-(trifluoromethoxy)phenyl)propanoyl)pyridin-2(1H)-one

To a solution of 1-(3-bromobenzyl)-5-(1-hydroxy-3-(((4-methoxybenzyl)oxy)imino)-3-(4-(trifluoromethoxy)phenyl)propyl)pyridin-2(1H)-one (112 mg, 0.17 mmol) in CH₂Cl₂ (2 mL) was added Dess-Martin periodinane (83 mg, 0.19 mmol) and the mixture was stirred at RT for 3 h. Na₂S₂O₃ (aq. sat., 2 mL) was added to the mixture and allowed to stir for 15 minutes, then diluted with CH₂Cl₂ (5 mL) and washed with H₂O (2×5 mL). The organic layer was washed with brine (5 mL), dried with Na₂SO₄, filtered and concentrated under reduced pressure. The residue was purified by SiO₂ gel chromatography (0% to 60% EtOAc in Hexanes) to give the title compound as a yellow oil (111 mg, 99%). MS(ES⁺) C₃₀H₂₄BrF₃N₂O₅ requires: 628. found: 629 [M+H]⁺.

### Step 5: 1-(3-Bromobenzyl)-5-(3-(4-(trifluoromethoxy)phenyl)isoxazol-5-yl)pyridin-2(1H)-one

To a suspension of 1-(3-bromobenzyl)-5-(3-(((4-methoxybenzyl)oxy)imino)-3-(4-(trifluoromethoxy)phenyl)propanoyl)pyridin-2(1H)-one (111 mg, 0.17 mmol) in CH₂Cl₂ (2 mL) at RT was added TFA (137 μL, 1.7 mmol). The mixture was then heated to 50° C. for 3 h, cooled to RT and concentrated under reduced pressure. The residue was purified by SiO₂ gel chromatography (20% to 100% EtOAc in Hexanes) to give the title compound as a white solid (48 mg, 55%). MS (ES⁺) C₂₂H₁₄BrF₃N₂O₃ requires: 491. found: 493 [M+H]⁺.

### Step 6: 1-(3-(4-(Methylsulfonyl)piperidin-1-yl)benzyl)-5-(3-(4-(trifluoromethoxy)phenyl)isoxazol-5-yl)pyridin-2(1H)-one

A suspension of 1-(3-bromobenzyl)-5-(3-(4-(trifluoromethoxy)phenyl)isoxazol-5-yl)pyridin-2(1H)-one (26 mg, 0.053 mmol), 4-(methylsulfonyl)piperidine (9.5 mg, 0.058 mmol), and Cs₂CO₃ (35 mg, 0.10 mmol) in toluene (600 μL) was degassed with argon for 5 minutes. Pd₂(dba)₃ (2.4 mg, 0.05 μmol) and dicyclohexyl(2′,4′,6′-triisopropyl-[1,1′-biphenyl]-2-yl)phosphine (5.0 mg, 0.2 mmol) were added and the mixture was degassed a second time with argon for 5 minutes, then heated to 140° C. for 18 h. The mixture was cooled to RT, diluted with EtOAc (5 mL), filtered through a pad of Celite and concentrated under reduced pressure. The residue was purified by prep-HPLC (Mobile phase: A=0.1% TFA/H₂O, B=0.1% TFA/MeCN; Gradient: B=40%-80% in 12 min; Column: C18) to give the title compound as a white solid; MS (ES+) C₂₈H₂₆F₃N₃O₅S requires: 573. found: 574[M+H]⁺; ¹H NMR (600 MHz, DMSO-d₆) δ 8.58 (d, J=2.5 Hz, 1H), 8.01-7.97 (m, 2H), 7.90 (dd, J=9.5, 2.6 Hz, 1H), 7.56 (d, J=8.3 Hz, 2H), 7.39 (s, 1H), 7.19 (t, J=7.9 Hz, 1H), 7.05 (m, 1H), 6.91 (dd, J=8.2, 2.4 Hz, 1H), 6.74 (d, J=7.5 Hz, 1H), 6.64 (d, J=9.5 Hz, 1H), 5.15 (s, 2H), 3.84 (bd, J=13.2 Hz, 2H), 3.28 (m, 1H), 2.95 (s, 3H), 2.75 (ddd, J=12.5, 12.3, 2.5 Hz, 2H), 2.08 (bd, J=11.9 Hz, 2H), 1.68 (m, 2H).

### EXAMPLE 99

### 1-(3-(4-(Methylsulfonyl)piperidin-1-yl)benzyl)-5-(5-(4-(trifluoromethoxy)phenyl)isoxazol-3-yl)pyridin-2(1H)-one

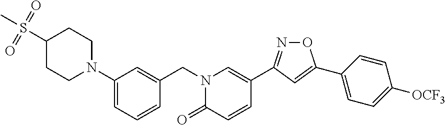 [SMILES: CS(=O)(=O)C1CCN(c2cccc(Cn3cc(-c4cc(-c5ccc(OC(F)(F)F)cc5)on4)ccc3=O)c2)CC1]

### Steps 1 to 6

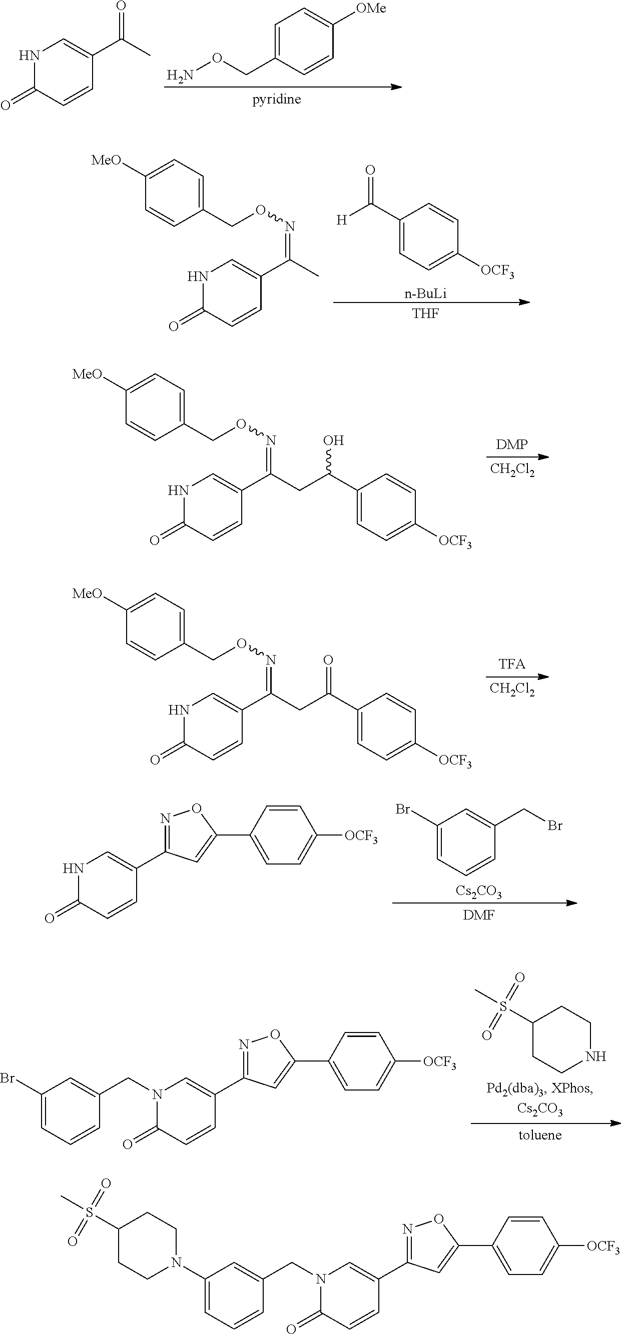

### Step 1: 5-(1-(((4-Methoxybenzyl)oxy)imino)ethyl)pyridin-2(1H)-one

To a suspension of 5-acetylpyridin-2(1H)-one (500 mg, 3.6 mmol) in pyridine (4 mL) was added 0-(4-methoxybenzyl)hydroxylamine hydrochloride (761 mg, 4.0 mmol) and the reaction mixture was stirred at RT for 18 h. The mixture was then partitioned between CH₂Cl₂ (10 mL) and H₂O (10 mL), the organic layer was washed with H₂O (2×10 mL), brine (2×10 mL), dried with Na₂SO₄, filtered and concentrated under reduced pressure. The residue was triturated with toluene to give the title compound as a white solid (991 mg, 99%) MS(ES⁺) C₁₅H₁₆N₂O₃ requires: 272. found: 273 [M+H]⁺.

### Step 2: 5-(3-Hydroxy-1-(((4-methoxybenzyl)oxy)imino)-3-(4-(trifluoromethoxy)phenyl)propyl)pyridin-2(1H)-one

To a solution of 5-(1-(((4-methoxybenzyl)oxy)imino)ethyl)pyridin-2(1H)-one (150 mg, 0.55 mmol) in THF (5.5 mL) at −78° C. was added dropwise n-BuLi (2.5 M in hexane, 485 μL, 1.2 mmol) and the resulting mixture was stirred at −78° C. for 1 h. A solution of 4-(trifluoromethoxy)benzaldehyde (126 mg, 0.66 mmol) in THF (1 mL) was then added dropwise and the reaction mixture was stirred at −78° C. for further 6 h. NH₄Cl (aq. sat., 5 mL) was added and the mixture was allowed to warm to RT, diluted with EtOAc (20 mL) and washed with H₂O (2×20 mL). The organic layer was washed with brine (2×20 mL), dried with Na₂SO₄, filtered and concentrated under reduced pressure. The residue was purified by SiO₂ gel chromatography with (25% to 90% EtOAc in Hexanes) to give the title compound as a white solid (183 mg, 72%). MS(ES⁺) C₂₃H₂₁F₃N₂O₅ requires: 462. found: 463 [M+H]⁺.

### Step 3: 5-(1-(((4-Methoxybenzyl)oxy)imino)-3-oxo-3-(4-(trifluoromethoxy)phenyl)propyl)pyridin-2(1H)-one

To a solution of 5-(3-hydroxy-1-(((4-methoxybenzyl)oxy)imino)-3-(4-(trifluoromethoxy)phenyl)propyl)pyridin-2(1H)-one (180 mg, 0.39 mmol) in CH₂Cl₂ (4 mL) was added Dess-Martin periodinane (182 mg, 0.42 mmol) and the mixture was stirred at RT for 3 h. Na₂S₂O₃ (aq. sat., 2 mL) was added and the mixture was stirred for further 15 minutes, then diluted with CH₂Cl₂ (10 mL) and washed with H₂O (2×10 mL). The organic layer was washed with brine (10 mL), dried with Na₂SO₄, filtered and concentrated under reduced pressure. The residue was purified by SiO₂ gel chromatography (20% to 100% EtOAc in Hexanes) to give the title compound as an off-white solid (110 mg, 62%). MS(ES⁺) C₂₃H₁₉F₃N₂O₅ requires: 460. found: 461 [M+H]⁺.

### Step 4: 5-(5-(4-(Trifluoromethoxy)phenyl)isoxazol-3-yl)pyridin-2(1H)-one

To a suspension of 5-(1-(((4-methoxybenzyl)oxy)imino)-3-oxo-3-(4-(trifluoromethoxy)phenyl)propyl)pyridin-2(1H)-one (110 mg, 0.24 mmol) in CH₂Cl₂ (3 mL) at RT was added TFA (184 μL, 2.3 mmol), and the mixture was heated to 50° C. for 3 h. After cooling to RT the mixture was concentrated under reduced pressure and the residue was purified by SiO₂ gel chromatography (0% to 10% MeOH in DCM) to give the title compound as a white solid (20 mg, 26%). MS(ES⁺) C₁₅H₉F₃N₂O₃ requires: 322. found: 323[M+H]⁺.

### Step 5: 1-(3-Bromobenzyl)-5-(5-(4-(trifluoromethoxy)phenyl)isoxazol-3-yl)pyridin-2(1H)-one

To a mixture of 5-(5-(4-(trifluoromethoxy)phenyl)isoxazol-3-yl)pyridin-2(1H)-one (20 mg, 0.062 mmol), 1-bromo-3-(bromomethyl)benzene (31 mg, 0.12 mmol), and LiCl (5.2 mg, 0.12 mmol) in DMF (700 μL) at RT was added Cs₂CO₃ (30 mg, 0.093 mmol) and the mixture was heated to 65° C. for 18 h. Upon cooling to RT, the mixture was partioned between EtOAc (10 mL) and H₂O (10 mL). The organic layer was washed with H₂O (3×10 mL), brine (10 mL), dried with Na₂SO₄, filtered and concentrated under reduced pressure. The residue was purified by SiO₂ gel chromatography (20% to 70% EtOAc in Hexanes) to give the title compound as a white solid (17 mg, 56%). MS(ES⁺) C₂₂H₁₄BrF₃N₂O₃ requires: 491. found: 493 [M+H]⁺.

### Step 6: 1-(3-(4-(Methylsulfonyl)piperidin-1-yl)benzyl)-5-(5-(4-(trifluoromethoxy)phenyl)isoxazol-3-yl)pyridin-2(1H)-one

A suspension of 1-(3-bromobenzyl)-5-(5-(4-(trifluoromethoxy)phenyl)isoxazol-3-yl)pyridin-2(1H)-one (7 mg, 0.014 mmol), 4-(methylsulfonyl)piperidine (2.5 mg, 0.016 mmol) and Cs₂CO₃ (9.2 mg, 0.028 mmol) in toluene (300 μL) was degassed with argon for 5 minutes. Pd₂(dba)₃ (0.65 mg, 0.71 μmol) and dicyclohexyl(2′,4′,6′-triisopropyl-[1,1′-biphenyl]-2-yl)phosphine (1.3 mg, 2.8 mmol) were added and the mixture was degassed a second time with argon for 5 minutes, then heated to 140° C. for 18 h. The mixture was cooled to RT, diluted with EtOAc (5 mL), filtered through a pad of Celite and concentrated under reduced pressure. The residue was purified by prep-HPLC (Mobile phase: A=0.1% TFA/H₂O, B=0.1% TFA/MeCN; Gradient: B=40%-80% in 12 min minutes; Column: C18) to give the title compound as a white solid; MS(ES⁺) C₂₈H₂₆F₃N₃O₅S requires: 573. found: 574 [M+H]⁺; ¹H NMR (600 MHz, DMSO-d₆) δ 8.52 (d, J=2.5 Hz, 1H), 8.02-7.96 (m, 2H), 7.92 (dd, J=9.5, 2.6 Hz, 1H), 7.62-7.55 (m, 2H), 7.51 (s, 1H), 7.20 (m, 1H), 7.05 (m, 1H), 6.93 (m, 1H), 6.74 (d, J=7.5 Hz, 1H), 6.61 (d, J=9.5 Hz, 1H), 5.14 (s, 2H), 3.83 (bd, J=13.5 Hz, 2H), 3.29 (m, 1H), 2.95 (s, 3H), 2.78 (bt, J=12.5 Hz, 2H), 2.08 (bd, J=10.5 Hz, 2H), 1.69 (m, 2H).

### EXAMPLE 100

### 1-Methyl-4-(3-((2-oxo-5-(5-(4-(trifluoromethoxy)phenyl)-1,3,4-oxadiazol-2-yl)pyridin-1(2H)-yl)methyl)phenyl)piperazin-1-ium 2,2,2-trifluoroacetate

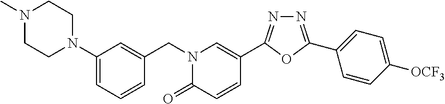 [SMILES: CN1CCN(c2cccc(Cn3cc(-c4nnc(-c5ccc(OC(F)(F)F)cc5)o4)ccc3=O)c2)CC1]

### Steps 1 to 2

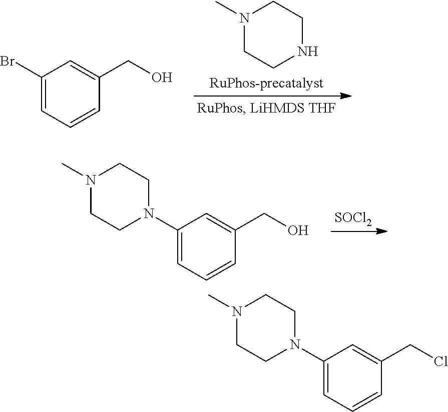

### Step 1: (3-(4-Methylpiperazin-1-yl)phenyl)methanol

To a solution of (3-bromophenyl)methanol (570 mg, 3.05 mmol) in THF (5 mL) was added LHMDS (7.43 mL, 7.43 mmol) and the mixture was stirred at RT for 30 minutes. Ru-phos precatalyst (97 mg, 0.133 mmol), dicyclohexyl(2′,6′-diisopropoxy-[1,1′-biphenyl]-2-yl)phosphine (61.9 mg, 0.133 mmol), and 1-methylpiperazine (0.441 mL, 3.98 mmol) were added, the mixture was degassed with a stream of nitrogen and heated to 75° C. for 1 h. The mixture was then diluted with DCM and washed with water. The organic layer was concentrated under reduced pressure and the residue was purified by SiO₂ gel chromatography (0-20% MeOH in DCM) to give the title compound (506 mg, 80%). MS(ES⁺) C₁₂H₁₈N₂O requires: 206. found: 207 [M+H]⁺.

### Step 2: 1-(3-(Chloromethyl)phenyl)-4-methylpiperazine

A mixture of thionyl chloride (87 mg, 0.727 mmol) and (3-(4-methylpiperazin-1-yl)phenyl)methanol (30.0 mg, 0.145 mmol) in DCM (10 mL) was heated to 60° C. for 2 h. The mixture was then concentrated under reduced pressure to a foam, which was used for the next step without further purification. MS(ES⁺) C₁₂H₁₈N₂O requires: 224. found: 225 [M+H]⁺.

### Steps 3 to 4

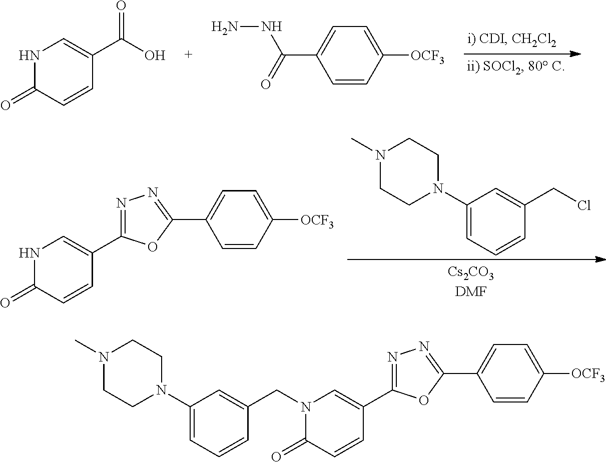

### Step 3: 5-(5-(4-(Trifluoromethoxy)phenyl)-1,3,4-oxadiazol-2-yl)pyridin-2(1H)-one

A mixture of di(1H-imidazol-1-yl)methanone (177 mg, 1.090 mmol) and 6-oxo-1,6-dihydropyridine-3-carboxylic acid (139 mg, 0.999 mmol) in DCM (3 ml) was stirred at 40° C. for 1 h. 4-(trifluoromethoxy)benzohydrazide (200 mg, 0.908 mmol) was then added and the mixture was stirred at RT for 8 h. The resulting suspension was filtered and the solid product was dissolved in thionyl chloride (3.30 ml, 45.4 mmol) and heated to 80° C. for 1 h. The mixture was concentrated under reduced pressure and the residue was dissolved in a small amount of DMF. The mixture was then diluted with DCM resulting in the precipitation of the title compound as a solid (138 mg, 47%). MS(ES⁺) C₁₄H₈F₃N₃O₃ requires: 323 found: 324 [M+H]⁺.

### Step 4: 1-Methyl-4-(3-((2-oxo-5-(5-(4-(trifluoromethoxy)phenyl)-1,3,4-oxadiazol-2-yl)pyridin-1(2H)-yl)methyl)phenyl)piperazin-1-ium 2,2,2-trifluoroacetate

A mixture of 5-(5-(4-(trifluoromethoxy)phenyl)-1,3,4-oxadiazol-2-yl)pyridin-2(1H)-one (23 mg, 0.0714 mmol), Cs₂CO₃ (349 mg, 1.071 mmol) and 1-(3-(chloromethyl)phenyl)-4-methylpiperazine (48.1 mg, 0.214 mmol) in DMF (3.0 ml) was stirred at RT for 2 h. The mixture was then diluted with water and extracted with EtOAc. The organic layer was concentrated under reduced pressure and purified by prep HPLC to give the title compound; MS(ES⁺) C₂₆H₂₄F₃N₅O₃ requires: 511. found: 512 [M+H]⁺; ¹H-NMR (600 MHz, CD₃OD) δ ppm 8.69 (d, J=2.4 Hz, 1H), 8.24 (d, J=9.0 Hz, 2H), 8.15 (dd, J=2.3, 9.5 Hz, 1H), 7.51 (d, J=9.1 Hz, 2H), 7.31 (m, 1H), 7.10 (s, 1H), 6.99 (dd, J=2.3, 8.5 Hz, 1H), 6.97 (d, J=7.3 Hz, 1H), 6.74 (d, J=9.3 Hz, 1H), 5.27 (s, 2H), 3.86 (d, J=13.2 Hz, 2H), 3.69 (d, J=13.2 Hz, 2H), 3.25 (m, 2H), 3.05 (m, 2H), 2.96 (s, 3H).

### EXAMPLE 101

### 1-Methyl-4-(4-((2-oxo-5-(5-(4-(trifluoromethoxy)phenyl)-1,3,4-oxadiazol-2-yl)pyridin-1(2H)-yl)methyl)pyridin-2-yl)piperazin-1-ium 2,2,2-trifluoroacetate

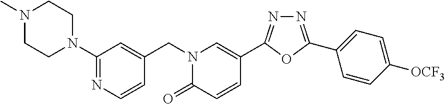 [SMILES: CN1CCN(c2cc(Cn3cc(-c4nnc(-c5ccc(OC(F)(F)F)cc5)o4)ccc3=O)ccn2)CC1]

Synthetized with analogous method as Example 7, using 5-(5-(4-(trifluoromethoxy)phenyl)-1,3,4-oxadiazol-2-yl)pyridin-2(1H)-one (Example 100, Step 3) instead of 5-(3-(4-(trifluoromethoxy)phenyl)-1,2,4-oxadiazol-5-yl)pyridin-2(1H)-one; MS(ES⁺) C₂₅H₂₃F₃N₆O₃ requires: 512. found: 513 [M+H]⁺; ¹H-NMR (600 MHz, CD₃OD) δ ppm 8.76 (d, J=2.4 Hz, 1H), 8.22 (d, J=9.0 Hz, 2H), 8.18 (dd, J=2.5, 9.6 Hz, 1H), 8.11 (d, J=5.4 Hz, 1H), 7.49 (d, J=8.5 Hz, 2H), 7.15 (s, 1H), 6.87 (d, J=5.2 Hz, 1H), 6.75 (d, J=9.5 Hz, 1H), 5.33 (s, 2H), 3.11-4.15 (m, 8H), 2.96 (s, 3H).

### EXAMPLE 102

### 2-{[3-(2-Hydroxyethoxy)phenyl]methyl}-6-{3-[4-(trifluoromethoxy)phenyl]-1,2,4-oxadiazol-5-yl}-2,3-dihydropyridazin-3-one

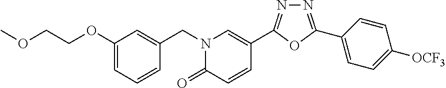 [SMILES: COCCOc1cccc(Cn2cc(-c3nnc(-c4ccc(OC(F)(F)F)cc4)o3)ccc2=O)c1]

Synthesized from 5-(5-(4-(trifluoromethoxy)phenyl)-1,3,4-oxadiazol-2-yl)pyridin-2(1H)-one (Example 100, Step 1) in an analogous method to Example 33. MS(ES⁺) C₂₄H₂₀F₃N₃O₅ requires: 487. found: 488 [M+H]⁺; ¹H-NMR (600 MHz, CDCl₃) δ ppm 8.17 (d, J=2.4 Hz, 1H), 8.12 (d, J=9.0 Hz, 2H), 7.98 (dd, J=2.3, 9.5 Hz, 1H), 7.37 (d, J=9.1 Hz, 2H), 7.29 (m, 1H), 6.93 (m, 2H), 6.89 (d, J=7.3 Hz, 1H), 6.76 (d, J=9.3 Hz, 1H), 5.20 (s, 2H), 4.15 (d, J=13.2 Hz, 2H), 3.74 (d, J=13.2 Hz, 2H), 3.43 (s, 3H).

### EXAMPLE 103

### 2-{[3-(2-Hydroxyethoxy)phenyl]methyl}-6-{3-[4-(trifluoromethoxy)phenyl]-1,2,4-oxadiazol-5-yl}-2,3-dihydropyridazin-3-one

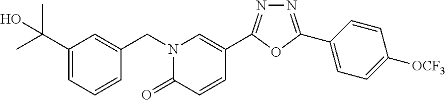 [SMILES: CC(C)(O)c1cccc(Cn2cc(-c3nnc(-c4ccc(OC(F)(F)F)cc4)o3)ccc2=O)c1]

Synthesized from 5-(5-(4-(trifluoromethoxy)phenyl)-1,3,4-oxadiazol-2-yl)pyridin-2(1H)-one (Example 100, Step 3) and 2-(3-(bromomethyl)phenyl)propan-2-ol (Example 39, Step 1) in an analogous method to Example 26. MS(ES⁺) C₂₄H₂₀F₃N₃O₄ requires: 471. found: 472 [M+H]⁺; ¹H-NMR (600 MHz, CDCl₃) δ ppm 8.11 (d, J=2.6 Hz, 1H), 8.09 (d, J=9.0 Hz, 2H), 7.97 (dd, J=2.3, 9.5 Hz, 1H), 7.54 (m, 1H), 7.45 (m, 1H), 7.36 (m, 3H), 7.21 (d, J=7.9 Hz, 1H), 6.77 (d, J=10 Hz, 1H), 5.26 (s, 2H), 1.75 (s, 1H), 1.58 (s, 6H).

### EXAMPLE 104

### 2-{[3-(2-Hydroxyethoxy)phenyl]methyl}-6-{3-[4-(trifluoromethoxy)phenyl]-1,2,4-oxadiazol-5-yl}-2,3-dihydropyridazin-3-one

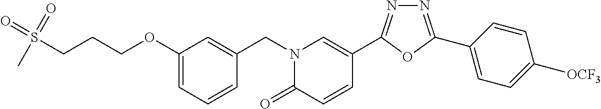 [SMILES: CS(=O)(=O)CCCOc1cccc(Cn2cc(-c3nnc(-c4ccc(OC(F)(F)F)cc4)o3)ccc2=O)c1]

Synthesized from 5-(5-(4-(trifluoromethoxy)phenyl)-1,3,4-oxadiazol-2-yl)pyridin-2(1H)-one (Example 100, Step 3) in an analogous method to Example 69. MS(ES⁺) C₂₅H₂₂F₃N₃O₆S: 549. found: 550 [M+H]⁺; ¹H-NMR (600 MHz, CDCl₃) δ ppm 8.22 (d, J=2.4 Hz, 1H), 8.11 (d, J=9.0 Hz, 2H), 7.98 (dd, J=2.3, 9.5 Hz, 1H), 7.36 (d, J=9.1 Hz, 2H), 7.30 (m, 1H), 6.95 (d, J=9.0 Hz, 1H), 6.89 (m, 1H), 6.85 (m, 1H), 6.77 (d, J=9.0 Hz, 1H), 5.21 (s, 2H), 4.11 (t, J=6.3 Hz, 2H), 3.25 (t, J=6.5 Hz, 2H), 2.94 (t, 3H), 2.33 (m, 2H).

### EXAMPLE 105

### 2-{[3-(2-Hydroxyethoxy)phenyl]methyl}-6-{3-[4-(trifluoromethoxy)phenyl]-1,2,4-oxadiazol-5-yl}-2,3-dihydropyridazin-3-one

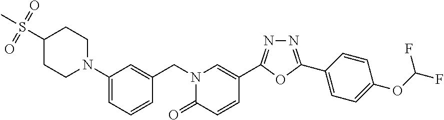 [SMILES: CS(=O)(=O)C1CCN(c2cccc(Cn3cc(-c4nnc(-c5ccc(OC(F)F)cc5)o4)ccc3=O)c2)CC1]

### Steps 1 to 4

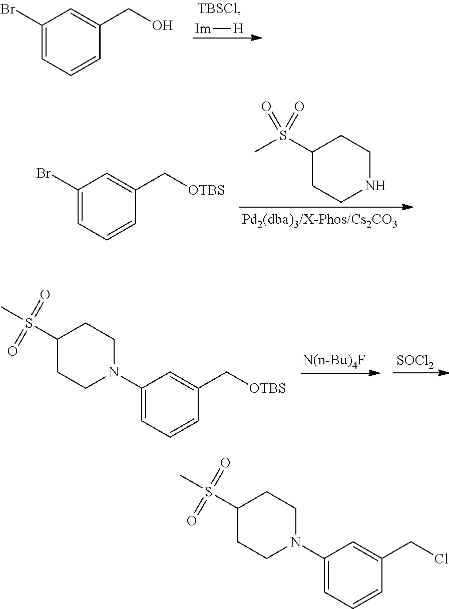

### Step 1: ((3-Bromobenzyl)oxy)(tert-butyl)dimethylsilane

A mixture of (3-bromophenyl)methanol (1.0 g, 5.35 mmol), imidazole (728 mg, 10.69 mmol) and tert-butyldimethylchlorosilane (1.21 g, 8.02 mmol) in DMF (10 mL) was stirred at RT for 16 h. The mixture was diluted with EtOAc and washed with water. The organic layer was dried over Na₂SO₄, filtered, concentrated under reduced pressure and purified by SiO₂ gel chromatography (0% to 20% EtOAc in Hexanes) to give ((3-bromobenzyl)oxy)(tert-butyl)dimethylsilane (1.6 g, 99%). MS(ES⁺) C₁₃H₂₁BrOSi requires: 301. found: 301/303 [M+H]⁺.

### Step 2: 1-(3-(((Tert-butyldimethylsilyl)oxy)methyl)phenyl)-4-(methylsulfonyl)piperidine

Synthesized in an analogous manner to Example 31, Step 2; 818 mg, 70%. MS(ES⁺) C₁₉H₃₃NO₃SSi requires: 383. found: 384 [M+H]⁺.

### Step 3: (3-(4-(Methylsulfonyl)piperidin-1-yl)phenyl)methanol

To a solution of 1-(3-(((tert-butyldimethylsilyl)oxy)methyl)phenyl)-4-(methylsulfonyl)piperidine (200 mg, 0.52 mmol) in THF (3 mL) was added TBAF (1M in THF, 0.52 mL, 0.52 mmol). The mixture was stirred at RT for 3 h and then concentrated under reduced pressure. The residue was purified by SiO₂ gel chromatography (0% to 10% MeOH in EtOAc) to give the title compound (130 mg, 93%). MS(ES⁺) C₁₃H₁₉NO₃S requires: 269. found: 270 [M+H]⁺.

### Step 4: 1-(3-(Chloromethyl)phenyl)-4-(methylsulfonyl)piperidine

To a solution of (3-(4-(Methylsulfonyl)piperidin-1-yl)phenyl)methanol (30 mg, 0.11 mmol) in DCM (1 mL) was added thionyl chloride (0.04 mL, 66 mmol). The mixture was stirred at RT for 1 h. The volatiles were removed under reduced pressure to give 1-(3-(chloromethyl)phenyl)-4-(methylsulfonyl)piperidine (32 mg, 100%), which was used without further purification in the next step. MS(ES⁺) C₁₃H₁₈ClNO₂S requires: 287. found: 288/290 [M+H]⁺.

### Step 5

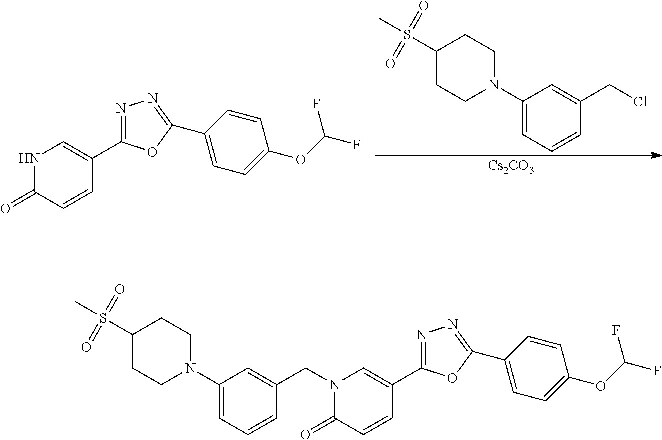 [SMILES: O=c1ccc(-c2nnc(-c3ccc(OC(F)F)cc3)o2)c[nH]1 ; CS(=O)(=O)C1CCN(c2cccc(CCl)c2)CC1 ; "Cs2CO3" ; CS(=O)(=O)C1CCN(c2cccc(Cn3cc(-c4nnc(-c5ccc(OC(F)F)cc5)o4)ccc3=O)c2)CC1]

### 5-(5-(4-(Difluoromethoxy)phenyl)-1,3,4-oxadiazol-2-yl)-1-(3-(4-(methylsulfonyl)piperidin-1-yl)benzyl)pyridin-2(1H)-one

Synthesized in an analogous manner to Example 26 using 5-(5-(4-(difluoromethoxy)phenyl)-1,3,4-oxadiazol-2-yl)pyridin-2(1H)-one (synthetized in analogous manner to Example 100, Step 3); MS(ES⁺) C₂₇H₂₆F₂N₄O₅S requires: 556. found: 557 [M+H]⁺; ¹H NMR (600 MHz, CDCl₃) δ 8.23 (s, 1H), 8.07 (d, J=8.4 Hz, 2H), 7.97 (d, J=9.6 Hz, 1H), 7.27 (m, 3H), 6.96 (s, 1H), 6.90 (d, J=8.4 Hz, 1H), 6.85 (d, J=8.4 Hz, 1H), 6.75 (d, J=9.6 Hz, 1H), 6.62 (m, 1H), 5.19 (s, 2H), 3.84 (m, 2H), 2.97 (m, 1H), 2.86 (s, 3H), 2.79 (m, 2H), 2.24 (m, 2H), 1.96 (m, 2H).

### EXAMPLE 106

### 6-(5-(4-Isopropoxyphenyl)-1,3,4-oxadiazol-2-yl)-2-(3-(4-(methylsulfonyl)piperidin-1-yl)benzyl)pyridazin-3(2H)-one

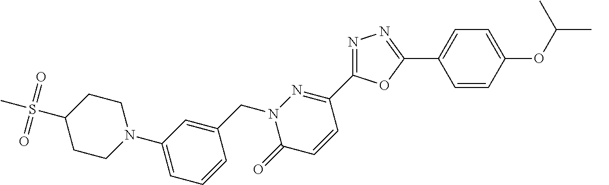 [SMILES: CC(C)Oc1ccc(-c2nnc(-c3ccc(=O)n(Cc4cccc(N5CCC(S(C)(=O)=O)CC5)c4)n3)o2)cc1]

Synthesized from 6-(5-(4-isopropoxyphenyl)-1,3,4-oxadiazol-2-yl)pyridazin-3(2H)-one (prepared in analogous manner to Example 100, Step 3) and 1-(3-(chloromethyl)phenyl)-4-(methylsulfonyl)piperidine in an analogous manner to Example 105. MS(ES⁺) C₂₈H₃₁N₅O₅S requires: 549. found 550 [M+H]⁺; ¹H NMR (600 MHz, CDCl₃) δ 8.12-8.05 (m, 3H), 7.44 (s, 1H), 7.40 (m, 1H), 7.32 (d, J=7.6 Hz, 1H), 7.23 (m, 1H), 7.11 (d, J=9.7 Hz, 1H), 7.05-7.00 (m, 2H), 5.44 (s, 2H), 4.68 (m, 1H), 3.95-3.86 (m, 2H), 3.19-3.11 (m, 2H), 3.08 (m, 1H), 2.90 (s, 3H), 2.51-2.42 (m, 2H), 2.35-2.24 (m, 2H), 1.39 (d, J=6.1 Hz, 6H).

The following compounds in Table 1 were synthesized and tested, and may generally be made by methods disclosed herein, and by methods known in the art.

TABLE 1

| Ex. | | | | | Ex. |
|---|---|---|---|---|---|
| No. | Structure | Name | MWt | [M + H] | Method |
| 107 | 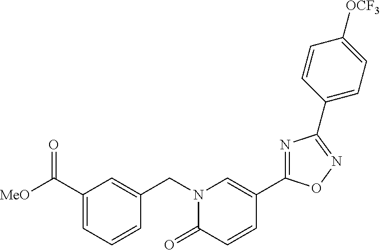 [SMILES: COC(=O)c1cccc(Cn2cc(-c3nc(-c4ccc(OC(F)(F)F)cc4)no3)ccc2=O)c1] | methyl 3- ((2-oxo-5-(3-(4- (trifluoromethoxy) phenyl)-1,2,4- oxadiazol-5- yl)pyridin-1(2H)- yl)methyl) benzoate | 471 | 472 | 26 |
| 108 | 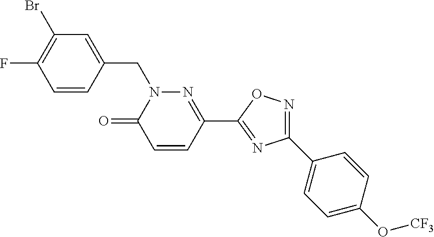 [SMILES: O=c1ccc(-c2nc(-c3ccc(OC(F)(F)F)cc3)no2)nn1Cc1ccc(F)c(Br)c1] | 2-(3-bromo-4- fluorobenzyl)- 6-(3-(4- (trifluoromethoxy) phenyl)-1,2,4- oxadiazol-5- yl)pyridazin- 3(2H)-one | 510/ 512 | 511/ 513 | 24 |
| 109 | 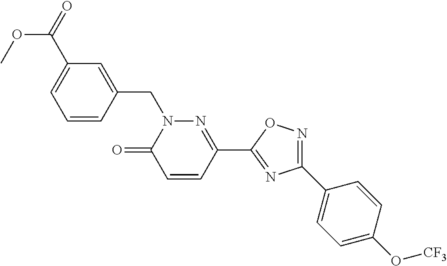 [SMILES: COC(=O)c1cccc(Cn2nc(-c3nc(-c4ccc(OC(F)(F)F)cc4)no3)ccc2=O)c1] | methyl 3- ((6-oxo-3-(3-(4- (trifluoromethoxy) phenyl)-1,2,4- oxadiazol-5- yl)pyridazin- 1(6H)-yl)methyl) benzoate | 472 | 473 | 24 |
| 110 | 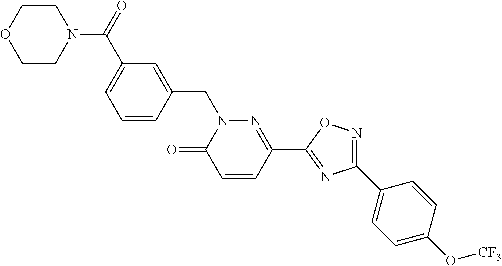 [SMILES: O=C(c1cccc(Cn2nc(-c3nc(-c4ccc(OC(F)(F)F)cc4)no3)ccc2=O)c1)N1CCOCC1] | 2-(3-(morpholine- 4-carbonyl) benzyl)-6-(3-(4- (trifluoromethoxy) phenyl)-1,2,4- oxadiazol-5- yl)pyridazin- 3(2H)-one | 527 | 528 | 25 |
| 111 | 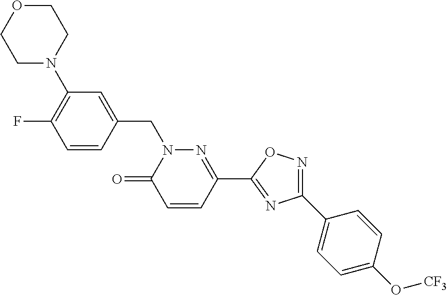 [SMILES: O=c1ccc(-c2nc(-c3ccc(OC(F)(F)F)cc3)no2)nn1Cc1ccc(F)c(N2CCOCC2)c1] | 2-(4-fluoro-3- morpholino benzyl)-6-(3-(4- (trifluoromethoxy) phenyl)-1,2,4- oxadiazol-5- yl)pyridazin- 3(2H)-one | 517 | 518 | 24 |
| 112 | 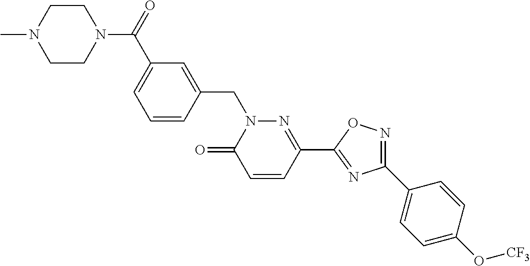 [SMILES: CN1CCN(C(=O)c2cccc(Cn3nc(-c4nc(-c5ccc(OC(F)(F)F)cc5)no4)ccc3=O)c2)CC1] | 2-(3-(4- methylpiperazine- 1-carbonyl) benzyl)-6-(3-(4- (trifluoromethoxy) phenyl)-1,2,4- oxadiazol-5- yl)pyridazin- 3(2H)-one | 510 | 511 | 19 |
| 113 | 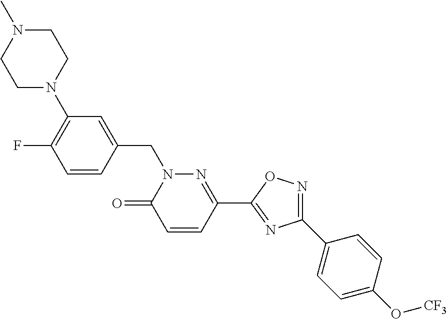 [SMILES: CN1CCN(c2cc(Cn3nc(-c4nc(-c5ccc(OC(F)(F)F)cc5)no4)ccc3=O)ccc2F)CC1] | 2-(4-fluoro-3-(4- methylpiperazin- 1-yl)benzyl)- 6-(3-(4- (trifluoromethoxy) phenyl)-1,2,4- oxadiazol-5- yl)pyridazin- 3(2H)-one | 530 | 531 | 13 |
| 114 | 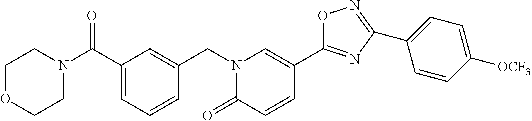 [SMILES: O=C(c1cccc(Cn2cc(-c3nc(-c4ccc(OC(F)(F)F)cc4)no3)ccc2=O)c1)N1CCOCC1] | 1-(3-(morpholine- 4-carbonyl) benzyl)-5-(3-(4- (trifluoromethoxy) phenyl)-1,2,4- oxadiazol-5- yl)pyridin-2(1H)- one | 526 | 527 | 25 |
| 115 | 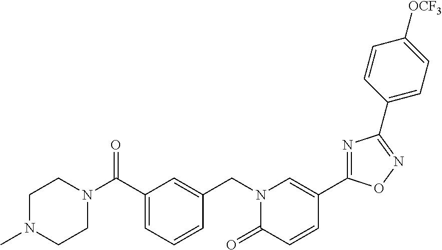 [SMILES: CN1CCN(C(=O)c2cccc(Cn3cc(-c4nc(-c5ccc(OC(F)(F)F)cc5)no4)ccc3=O)c2)CC1] | 1-(3-(4- methylpiperazine- 1-carbonyl) benzyl)-5-(3-(4- (trifluoromethoxy) phenyl)-1,2,4- oxadiazol-5- yl)pyridin-2(1H)- one | 539 | 540 | 25 |
| 116 | 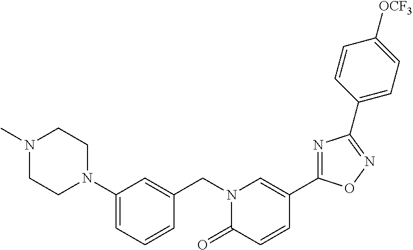 [SMILES: CN1CCN(c2cccc(Cn3cc(-c4nc(-c5ccc(OC(F)(F)F)cc5)no4)ccc3=O)c2)CC1] | 1-(3-(4- methylpiperazin- 1-yl)benzyl)- 5-(3-(4- (trifluoromethoxy) phenyl)-1,2,4- oxadiazol-5- yl)pyridin-2(1H)- one | 511 | 512 | 6 |
| 117 | 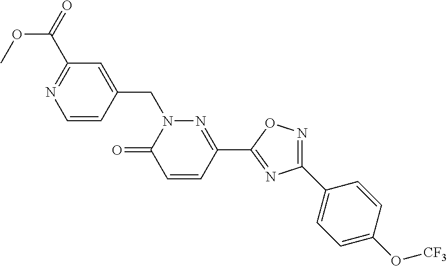 [SMILES: COC(=O)c1cc(Cn2nc(-c3nc(-c4ccc(OC(F)(F)F)cc4)no3)ccc2=O)ccn1] | methyl 4- ((6-oxo-3-(3-(4- (trifluoromethoxy) phenyl)-1,2,4- oxadiazol-5- yl)pyridazin- 1(6H)-yl)methyl) picolinate | 473 | 474 | 24 |
| 118 | 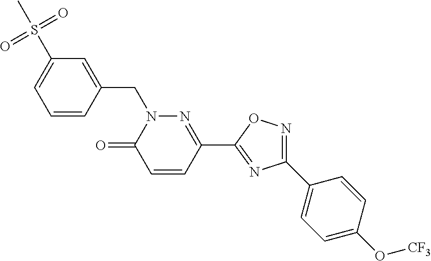 [SMILES: CS(=O)(=O)c1cccc(Cn2nc(-c3nc(-c4ccc(OC(F)(F)F)cc4)no3)ccc2=O)c1] | 2-(3- (methylsulfonyl) benzyl)-6-(3-(4- (trifluoromethoxy) phenyl)-1,2,4- oxadiazol-5- yl)pyridazin- 3(2H)-one | 492 | 493 | 24 |
| 119 | 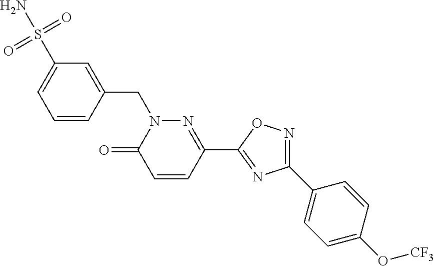 [SMILES: NS(=O)(=O)c1cccc(Cn2nc(-c3nc(-c4ccc(OC(F)(F)F)cc4)no3)ccc2=O)c1] | 3-((6-oxo-3-(3-(4- (trifluoromethoxy) phenyl)-1,2,4- oxadiazol-5- yl)pyridazin- 1(6H)-yl)methyl) benzenesulfon- amide | 493 | 494 | 24 |
| 120 | 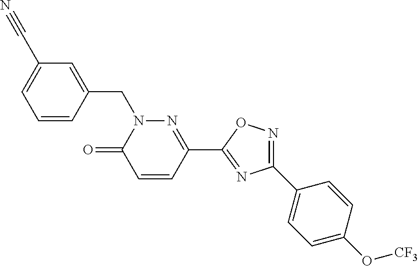 [SMILES: N#Cc1cccc(Cn2nc(-c3nc(-c4ccc(OC(F)(F)F)cc4)no3)ccc2=O)c1] | 3-((6-oxo-3-(3-(4- (trifluoromethoxy) phenyl)-1,2,4- oxadiazol-5- yl)pyridazin- 1(6H)-yl)methyl) benzonitrile | 439 | 440 | 24 |
| 121 | 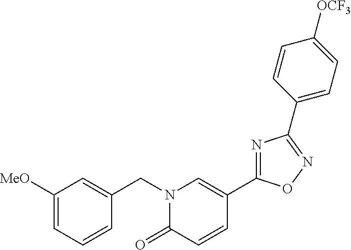 [SMILES: COc1cccc(Cn2cc(-c3nc(-c4ccc(OC(F)(F)F)cc4)no3)ccc2=O)c1] | 1-(3- methoxybenzyl)- 5-(3-(4- (trifluoromethoxy) phenyl)-1,2,4- oxadiazol-5- yl)pyridin-2(1H)- one | 443 | 444 | 26 |
| 122 | 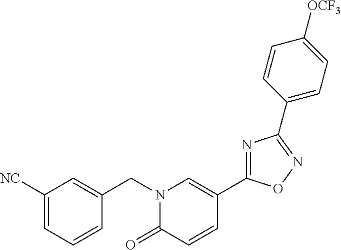 [SMILES: N#Cc1cccc(Cn2cc(-c3nc(-c4ccc(OC(F)(F)F)cc4)no3)ccc2=O)c1] | 3-((2-oxo-5-(3-(4- (trifluoromethoxy) phenyl)-1,2,4- oxadiazol-5- yl)pyridin-1(2H)- yl)methyl) benzonitrile | 438 | 439 | 26 |
| 123 | 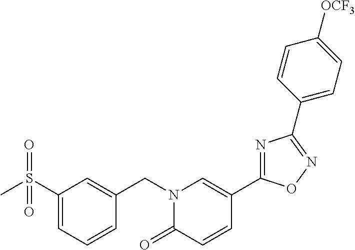 [SMILES: CS(=O)(=O)c1cccc(Cn2cc(-c3nc(-c4ccc(OC(F)(F)F)cc4)no3)ccc2=O)c1] | 1-(3- (methylsulfonyl) benzyl)-5-(3-(4- (trifluoromethoxy) phenyl)-1,2,4- oxadiazol-5- yl)pyridin-2(1H)- one | 491 | 492 | 26 |
| 124 | 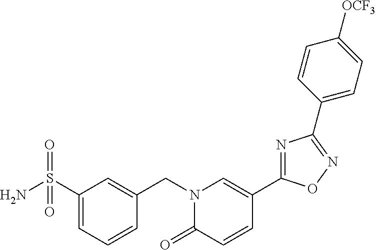 [SMILES: NS(=O)(=O)c1cccc(Cn2cc(-c3nc(-c4ccc(OC(F)(F)F)cc4)no3)ccc2=O)c1] | 3-((2-oxo-5-(3-(4- (trifluoromethoxy) phenyl)-1,2,4- oxadiazol-5- yl)pyridin-1(2H)- yl)methyl) benzenesulfon- amide | 492 | 493 | 26 |
| 125 | 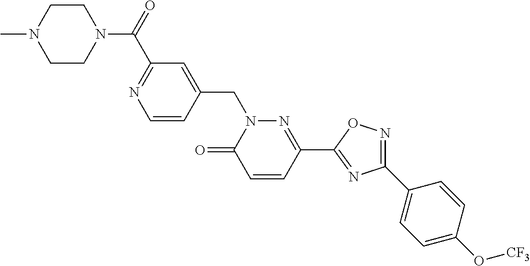 [SMILES: CN1CCN(C(=O)c2cc(Cn3nc(-c4nc(-c5ccc(OC(F)(F)F)cc5)no4)ccc3=O)ccn2)CC1] | 2-((2-(4- methylpiperazine- 1-carbonyl) pyridin-4-yl) methyl)-6-(3-(4- (trifluoromethoxy) phenyl)-1,2,4- oxadiazol-5- yl)pyridazin- 3(2H)-one | 541 | 542 | 25 |
| 126 | 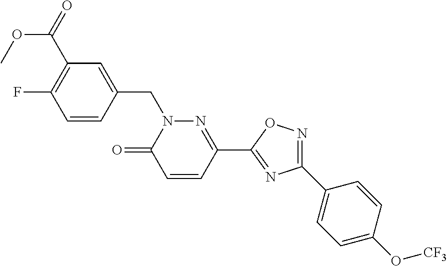 [SMILES: COC(=O)c1cc(Cn2nc(-c3nc(-c4ccc(OC(F)(F)F)cc4)no3)ccc2=O)ccc1F] | methyl 2-fluoro-5- ((6-oxo-3-(3-(4- (trifluoromethoxy) phenyl)-1,2,4- oxadiazol-5- yl)pyridazin- 1(6H)-yl)methyl) benzoate | 490 | 491 | 24 |
| 127 | 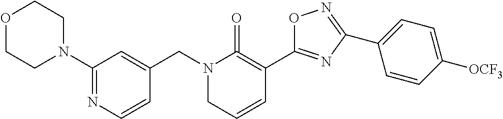 | 1-((2-morpholino pyridin-4-yl) methyl)-3-(3-(4- (trifluoromethoxy) phenyl)-1,2,4- oxadiazol-5- yl)pyridin-2(1H)- one | 499 | 500 | 20 |
| 128 | 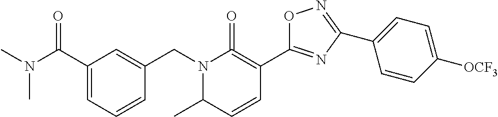 | N,N-dimethyl-3- ((6-methyl- 2-oxo-3-(3-(4- (trifluoromethoxy) phenyl)-1,2,4- oxadiazol-5- yl)pyridin-1(2H)- yl)methyl) benzamide | 498 | 499 | 23 |
| 129 | 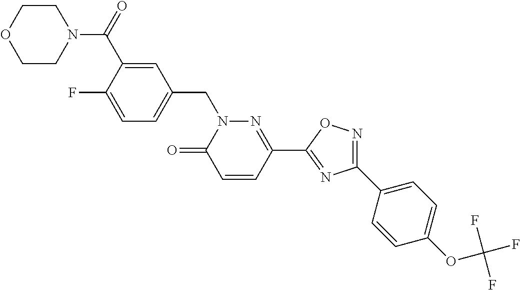 [SMILES: O=C(c1cc(Cn2nc(-c3nc(-c4ccc(OC(F)(F)F)cc4)no3)ccc2=O)ccc1F)N1CCOCC1] | 2-{[4-fluoro-3- (morpholine-4- carbonyl)phenyl] methyl}-6-{3-[4- (trifluoromethoxy) phenyl]-1,2,4- oxadiazol-5-yl}- 2,3-dihydro- pyridazin-3-one | 545 | 546 | 25 |
| 130 | 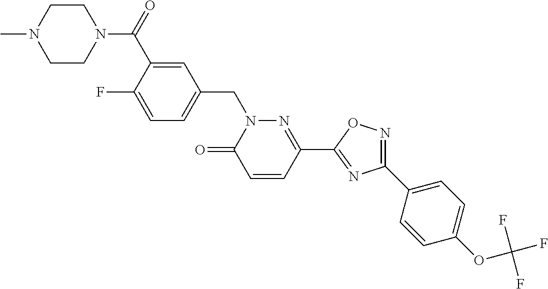 [SMILES: CN1CCN(C(=O)c2cc(Cn3nc(-c4nc(-c5ccc(OC(F)(F)F)cc5)no4)ccc3=O)ccc2F)CC1] | 2-{[4-fluoro-3-(4- methylpiperazine- 1-carbonyl) phenyl]methyl}- 6-{3-[4- (trifluoromethoxy) phenyl]-1,2,4- oxadiazol-5-yl}- 2,3-dihydro- pyridazin-3-one | 558 | 559 | 25 |
| 131 | 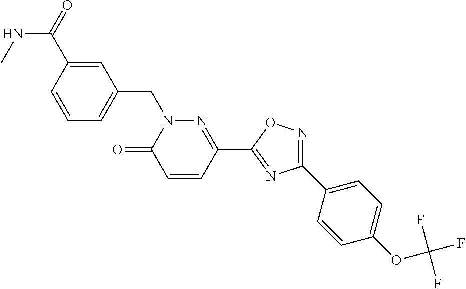 [SMILES: CNC(=O)c1cccc(Cn2nc(-c3nc(-c4ccc(OC(F)(F)F)cc4)no3)ccc2=O)c1] | N-methyl-3- [(6-oxo-3-{3-[4- (trifluoromethoxy) phenyl]-1 2 4- oxadiazol-5-yl}- 1 6-dihydro- pyridazin-1-yl) methyl]benzamide | 471 | 472 | 25 |
| 132 | 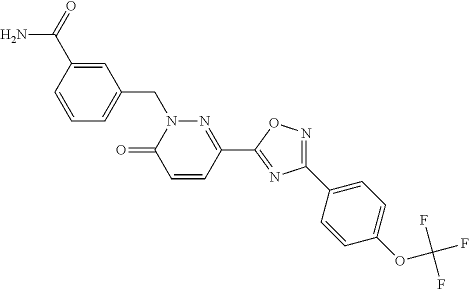 [SMILES: NC(=O)c1cccc(Cn2nc(-c3nc(-c4ccc(OC(F)(F)F)cc4)no3)ccc2=O)c1] | 3-[(6-oxo-3-{3-[4- (trifluoromethoxy) phenyl]-1,2,4- oxadiazol-5-yl}- 1,6-dihydro- pyridazin-1-yl) methyl]benzamide | 457 | 458 | 25 |
| 133 | 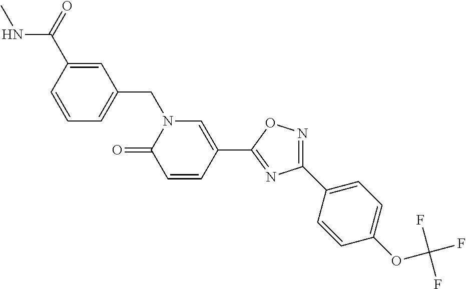 [SMILES: CNC(=O)c1cccc(Cn2cc(-c3nc(-c4ccc(OC(F)(F)F)cc4)no3)ccc2=O)c1] | N-methyl-3- [(2-oxo-5-{3-[4- (trifluoromethoxy) phenyl]-1,2,4- oxadiazol-5-yl}- 1,2-dihydro- pyridin-1-yl) methyl]benzamide | 470 | 471 | 25 |
| 134 | 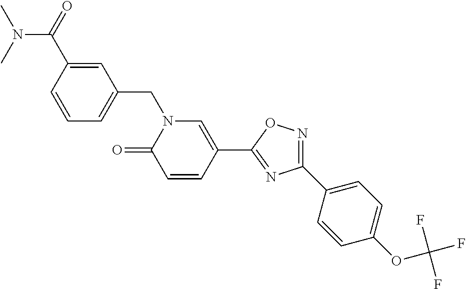 [SMILES: CN(C)C(=O)c1cccc(Cn2cc(-c3nc(-c4ccc(OC(F)(F)F)cc4)no3)ccc2=O)c1] | N,N-dimethyl-3- [(2-oxo-5-{3-[4- (trifluoromethoxy) phenyl]-1,2,4- oxadiazol-5-yl}- 1,2-dihydro- pyridin-1-yl) methyl]benzamide | 484 | 485 | 25 |
| 135 | 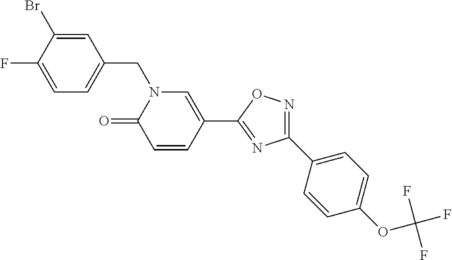 [SMILES: O=c1ccc(-c2nc(-c3ccc(OC(F)(F)F)cc3)no2)cn1Cc1ccc(F)c(Br)c1] | 1-[(3-bromo-4- fluorophenyl) methyl]-5-{3-[4- (trifluoromethoxy) phenyl]-1,2,4- oxadiazol-5-yl}- 1,2-dihydro- pyridin-2-one | 510 | 510; 512 | 26 |
| 136 | 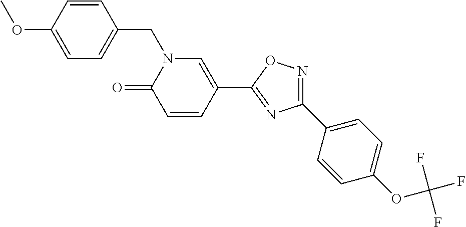 [SMILES: COc1ccc(Cn2cc(-c3nc(-c4ccc(OC(F)(F)F)cc4)no3)ccc2=O)cc1] | 1-[(4- methoxyphenyl) methyl]-5-{3-[4- (trifluoromethoxy) phenyl]-1,2,4- oxadiazol-5-yl}- 1,2-dihydro- pyridin-2-one | 443 | 444 | 26 |
| 137 | 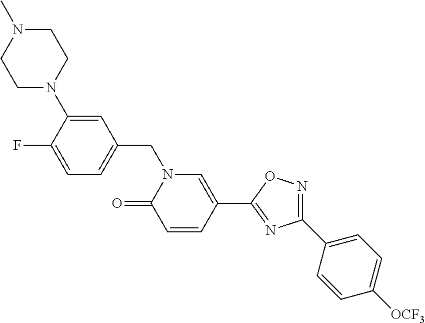 [SMILES: CN1CCN(c2cc(Cn3cc(-c4nc(-c5ccc(OC(F)(F)F)cc5)no4)ccc3=O)ccc2F)CC1] | 1-{[4-fluoro-3-(4- methylpiperazin- 1-yl)phenyl] methyl}-5-{3-[4- (trifluoromethoxy) phenyl]-1,2,4- oxadiazol-5-yl}- 1,2-dihydro- pyridin-2-one | 529 | 530 | 31 |
| 138 | 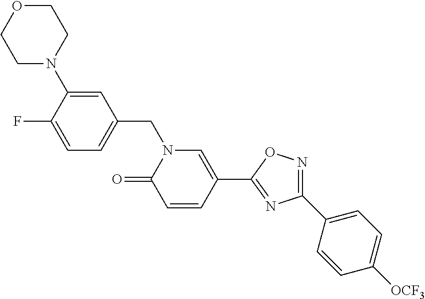 [SMILES: O=c1ccc(-c2nc(-c3ccc(OC(F)(F)F)cc3)no2)cn1Cc1ccc(F)c(N2CCOCC2)c1] | 1-{[4-fluoro-3- (morpholin-4-yl) phenyl]methyl}- 5-{3-[4- (trifluoromethoxy) phenyl]-1,2,4- oxadiazol-5-yl}- 1,2-dihydro- pyridin-2-one | 516 | 517 | 31 |
| 139 | 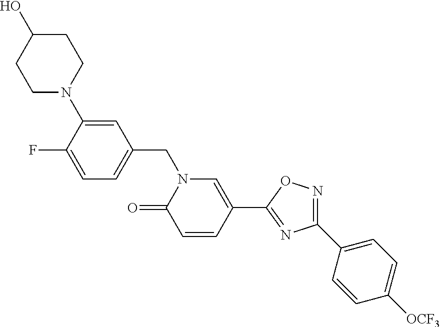 [SMILES: O=c1ccc(-c2nc(-c3ccc(OC(F)(F)F)cc3)no2)cn1Cc1ccc(F)c(N2CCC(O)CC2)c1] | 1-{[4-fluoro-3-(4- hydroxypiperidin- 1-yl)phenyl] methyl}-5-{3-[4- (trifluoromethoxy) phenyl]-1,2,4- oxadiazol-5-yl}- 1,2-dihydro- pyridin-2-one | 530 | 531 | 31 |
| 140 | 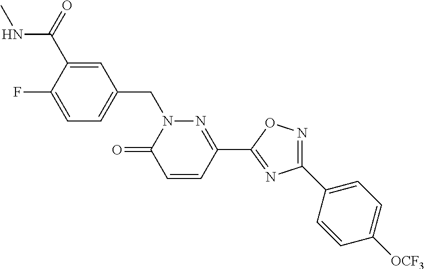 [SMILES: CNC(=O)c1cc(Cn2nc(-c3nc(-c4ccc(OC(F)(F)F)cc4)no3)ccc2=O)ccc1F] | 2-fluoro-N- methyl-5-[(6-oxo- 3-{3-[4- (trifluoromethoxy) phenyl]-1,2,4- oxadiazol-5-yl}- 1,6-dihydro- pyridazin-1-yl) methyl]benzamide | 489 | 490 | 25 |
| 141 | 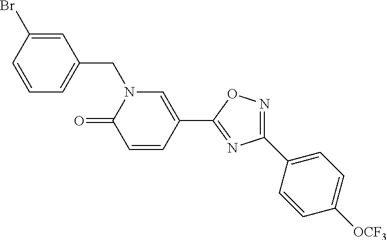 [SMILES: O=c1ccc(-c2nc(-c3ccc(OC(F)(F)F)cc3)no2)cn1Cc1cccc(Br)c1] | 1-[(3- bromophenyl) methyl]-5-{3-[4- (trifluoromethoxy) phenyl]-1,2,4- oxadiazol-5-yl}- 1,2-dihydro- pyridin-2-one | 492 | 492; 493 | 26 |
| 142 | 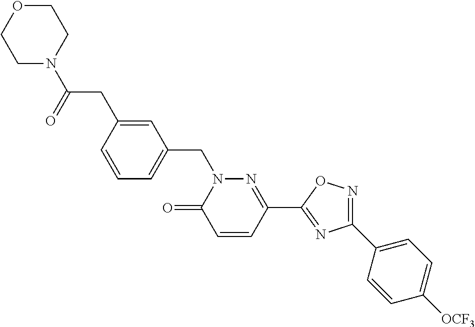 [SMILES: O=C(Cc1cccc(Cn2nc(-c3nc(-c4ccc(OC(F)(F)F)cc4)no3)ccc2=O)c1)N1CCOCC1] | 2-({3-[2- (morpholin-4-yl)- 2-oxoethyl] phenyl}methyl)- 6-{3-[4- (trifluoromethoxy) phenyl]-1,2,4- oxadiazol-5-yl}- 2,3-dihydro- pyridazin-3-one | 541 | 542 | 25 |
| 143 | 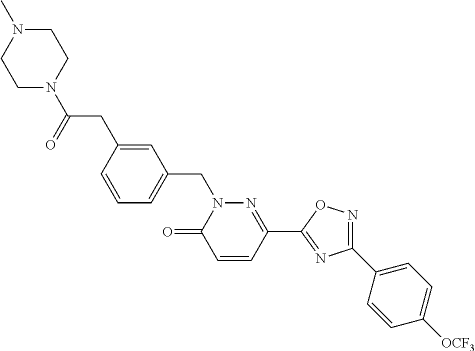 [SMILES: CN1CCN(C(=O)Cc2cccc(Cn3nc(-c4nc(-c5ccc(OC(F)(F)F)cc5)no4)ccc3=O)c2)CC1] | 2-({3-[2-(4- methylpiperazin- 1-yl)-2- oxoethyl]phenyl} methyl)-6-{3-[4- (trifluoromethoxy) phenyl]-1,2,4- oxadiazol-5-yl}- 2,3-dihydro- pyridazin-3-one | 555 | 556 | 25 |
| 144 | 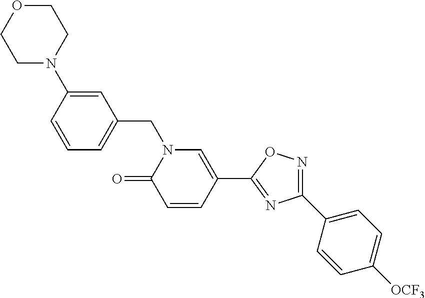 [SMILES: O=c1ccc(-c2nc(-c3ccc(OC(F)(F)F)cc3)no2)cn1Cc1cccc(N2CCOCC2)c1] | 1-{[3-(morpholin- 4-yl)phenyl] methyl}-5-{3-[4- (trifluoromethoxy) phenyl]-1,2,4- oxadiazol-5-yl}- 1,2-dihydro- pyridin-2-one | 498 | 499 | 31 |
| 145 | 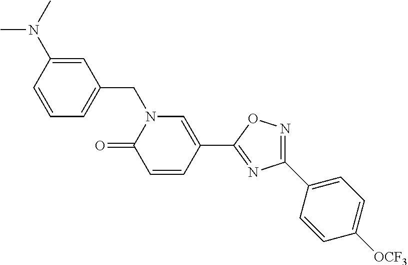 [SMILES: CN(C)c1cccc(Cn2cc(-c3nc(-c4ccc(OC(F)(F)F)cc4)no3)ccc2=O)c1] | 1-{[3- (dimethylamino) phenyl]methyl}- 5-{3-[4- (trifluoromethoxy) phenyl]-1,2,4- oxadiazol-5-yl}- 1,2-dihydro- pyridin-2-one | 456 | 457 | 26 |
| 146 | 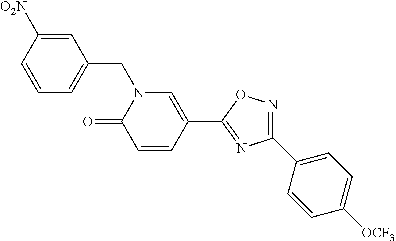 [SMILES: O=c1ccc(-c2nc(-c3ccc(OC(F)(F)F)cc3)no2)cn1Cc1cccc([N+](=O)[O-])c1] | 1-[(3-nitrophenyl) methyl]-5-{3-[4- (trifluoromethoxy) phenyl]-1,2,4- oxadiazol-5-yl}- 1,2-dihydro- pyridin-2-one | 458 | 459 | 26 |
| 147 | 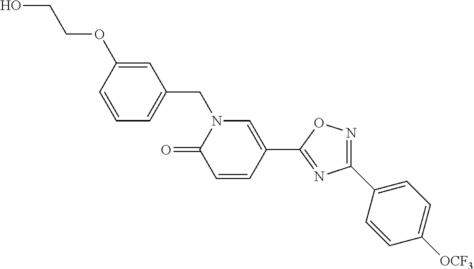 [SMILES: O=c1ccc(-c2nc(-c3ccc(OC(F)(F)F)cc3)no2)cn1Cc1cccc(OCCO)c1] | 1-{[3-(2- hydroxyethoxy) phenyl]methyl}- 5-{3-[4- (trifluoromethoxy) phenyl]-1,2,4- oxadiazol-5-yl}- 1,2-dihydro- pyridin-2-one | 473 | 474 | 33 |
| 148 | 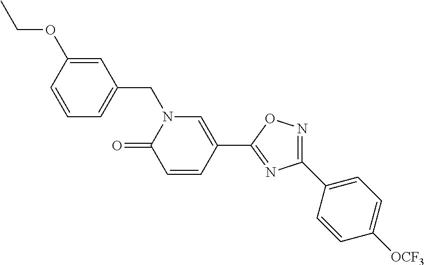 [SMILES: CCOc1cccc(Cn2cc(-c3nc(-c4ccc(OC(F)(F)F)cc4)no3)ccc2=O)c1] | 1-[(3- ethoxyphenyl) methyl]-5-{3-[4- (trifluoromethoxy) phenyl]-1,2,4- oxadiazol-5-yl}- 1,2-dihydro- pyridin-2-one | 457 | 458 | 33 |
| 149 | 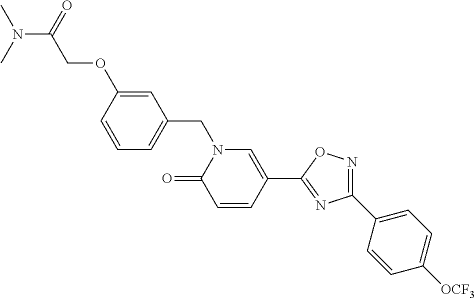 [SMILES: CN(C)C(=O)COc1cccc(Cn2cc(-c3nc(-c4ccc(OC(F)(F)F)cc4)no3)ccc2=O)c1] | N,N-dimethyl-2- {3-[(2-oxo- 5-{3-[4- (trifluoromethoxy) phenyl]-1,2,4- oxadiazol-5-yl}- 1,2-dihydro- pyridin-1-yl) methyl]phenoxy} acetamide | 514 | 515 | 33 |
| 150 | 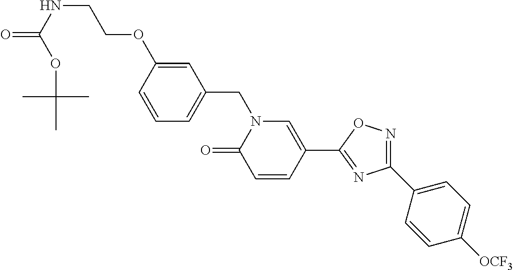 [SMILES: CC(C)(C)OC(=O)NCCOc1cccc(Cn2cc(-c3nc(-c4ccc(OC(F)(F)F)cc4)no3)ccc2=O)c1] | tert-butyl N-(2-{3- [(2-oxo-5-{3-[4- (trifluoromethoxy) phenyl]-1,2,4- oxadiazol-5-yl}- 1,2-dihydro- pyridin-1-yl) methyl]phenoxy} ethyl)carbamate | 573 | 574 | 33 |
| 151 | 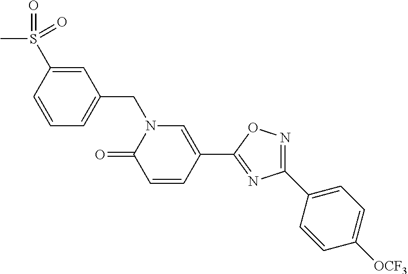 [SMILES: CS(=O)(=O)c1cccc(Cn2cc(-c3nc(-c4ccc(OC(F)(F)F)cc4)no3)ccc2=O)c1] | 1-[(4-methane- sulfonylphenyl) methyl]-5-{3-[4- (trifluoromethoxy) phenyl]-1,2,4- oxadiazol-5-yl}- 1,2-dihydro- pyridin-2-one | 491 | 492 | 26 |
| 152 | 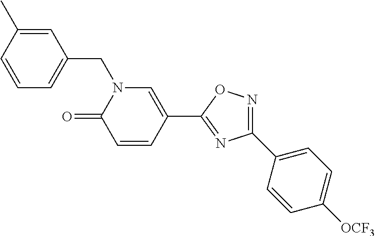 [SMILES: Cc1cccc(Cn2cc(-c3nc(-c4ccc(OC(F)(F)F)cc4)no3)ccc2=O)c1] | 1-[(3- methylphenyl) methyl]-5-{3-[4- (trifluoromethoxy) phenyl]-1,2,4- oxadiazo-5-yl}- 1,2-dihydro- pyridin-2-one | 427 | 428 | 26 |
| 153 | 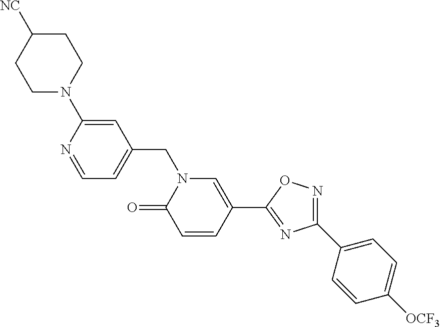 [SMILES: N#CC1CCN(c2cc(Cn3cc(-c4nc(-c5ccc(OC(F)(F)F)cc5)no4)ccc3=O)ccn2)CC1] | 1-{4-[(2- oxo-5-{3-[4- (trifluoromethoxy) phenyl]-1,2,4- oxadiazol-5-yl}- 1,2-dihydro- pyridin-1-yl) methyl]pyridin-2- yl}piperidine-4- carbonitrile | 522 | 523 | 7 |
| 154 | 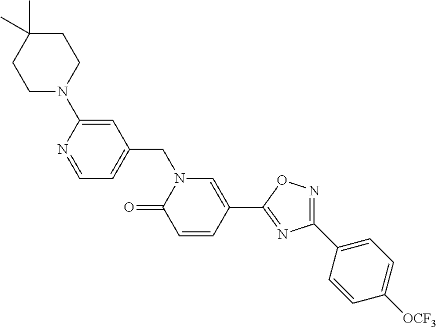 [SMILES: CC1(C)CCN(c2cc(Cn3cc(-c4nc(-c5ccc(OC(F)(F)F)cc5)no4)ccc3=O)ccn2)CC1] | 1-{[2-(4,4- dimethylpiperidin- 1-yl)pyridin-4-yl] methyl}-5-{3-[4- (trifluoromethoxy) phenyl]-1,2,4- oxadiazol-5-yl}- 1,2-dihydro- pyridin-2-one | 526 | 527 | 7 |
| 155 | 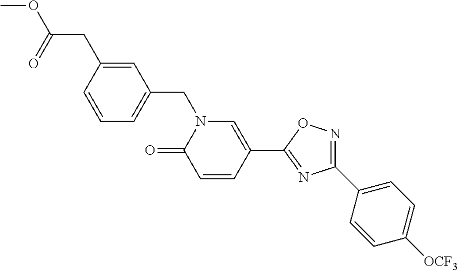 [SMILES: COC(=O)Cc1cccc(Cn2cc(-c3nc(-c4ccc(OC(F)(F)F)cc4)no3)ccc2=O)c1] | methyl 2-{3-[(2- oxo-5-{3-[4- (trifluoromethoxy) phenyl]-1,2,4- oxadiazol-5-yl}- 1,2-dihydro- pyridin-1-yl) methyl]phenyl} acetate | 485 | 486 | 29 |
| 156 | 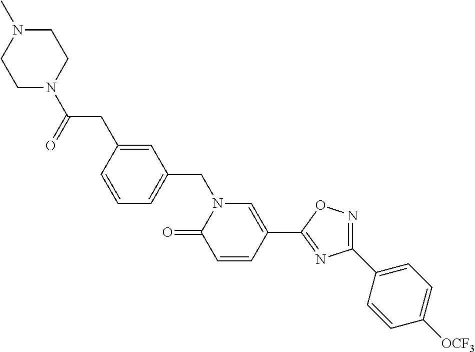 [SMILES: CN1CCN(C(=O)Cc2cccc(Cn3cc(-c4nc(-c5ccc(OC(F)(F)F)cc5)no4)ccc3=O)c2)CC1] | 1-({3-[2-(4- methylpiperazin- 1-yl)-2-oxoethyl] phenyl}methyl)- 5-{3-[4- (trifluoromethoxy) phenyl]-1,2,4- oxadiazol-5-yl}- 1,2-dihydro- pyridin-2-one | 554 | 555 | 25 |
| 157 | 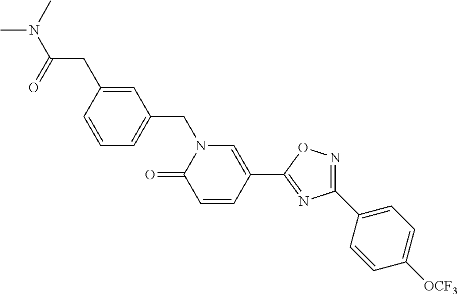 [SMILES: CN(C)C(=O)Cc1cccc(Cn2cc(-c3nc(-c4ccc(OC(F)(F)F)cc4)no3)ccc2=O)c1] | N,N-dimethyl-2- {3-[(2-oxo- 5-{3-[4- (trifluoromethoxy) phenyl]-1,2,4- oxadiazol-5-yl}- 1,2-dihydro- pyridin-1-yl) methyl]phenyl} acetamide | 498 | 499 | 25 |
| 158 | 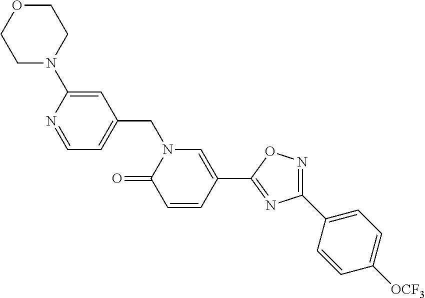 [SMILES: O=c1ccc(-c2nc(-c3ccc(OC(F)(F)F)cc3)no2)cn1Cc1ccnc(N2CCOCC2)c1] | 1-{[2-(morpholin- 4-yl)pyridin-4-yl] methyl}-5-{3-[4- (trifluoromethoxy) phenyl]-1,2,4- oxadiazol-5-yl}- 1,2-dihydro- pyridin-2-one | 499 | 500 | 7 |
| 159 | 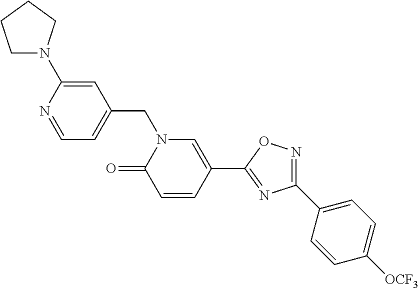 [SMILES: O=c1ccc(-c2nc(-c3ccc(OC(F)(F)F)cc3)no2)cn1Cc1ccnc(N2CCCC2)c1] | 1-{[2-(pyrrolidin- 1-yl)pyridin-4-yl] methyl}-5-{3-[4- (trifluoromethoxy) phenyl]-1,2,4- oxadiazol-5-yl}- 1,2-dihydro- pyridin-2-one | 483 | 484 | 7 |
| 160 | 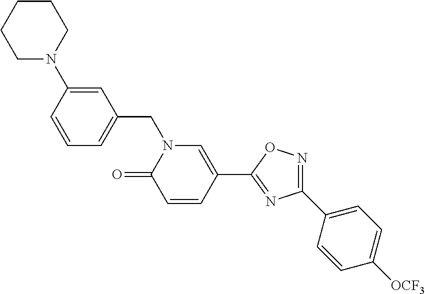 [SMILES: O=c1ccc(-c2nc(-c3ccc(OC(F)(F)F)cc3)no2)cn1Cc1cccc(N2CCCCC2)c1] | 1-{[3-(piperidin-1- yl)phenyl] methyl}-5-{3-[4- (trifluoromethoxy) phenyl]-1,2,4- oxadiazol-5-yl}- 1,2-dihydro- pyridin-2-one | 496 | 497 | 31 |
| 161 | 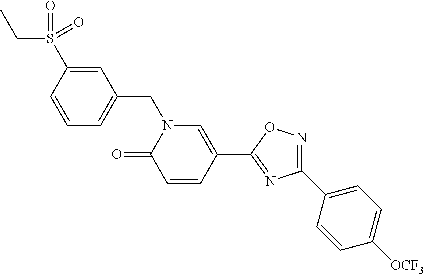 [SMILES: CCS(=O)(=O)c1cccc(Cn2cc(-c3nc(-c4ccc(OC(F)(F)F)cc4)no3)ccc2=O)c1] | 1-{[3- (ethanesulfonyl) phenyl]methyl}- 5-{3-[4- (trifluoromethoxy) phenyl]-1,2,4- oxadiazol-5-yl}- 1,2-dihydro- pyridin-2-one | 505 | 506 | 26 |
| 162 | 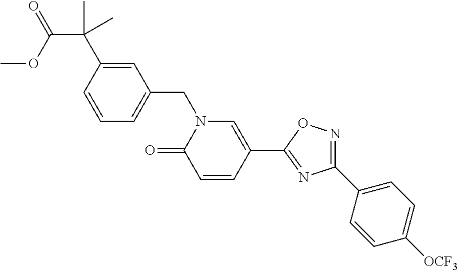 [SMILES: COC(=O)C(C)(C)c1cccc(Cn2cc(-c3nc(-c4ccc(OC(F)(F)F)cc4)no3)ccc2=O)c1] | methyl 2-methyl- 2-{3-[(2-oxo- 5-{3-[4- (trifluoromethoxy) phenyl]-1,2,4- oxadiazol-5-yl}- 1,2-dihydro- pyridin-1-yl) methyl]phenyl} propanoate | 513 | 514 | 30 |
| 163 | 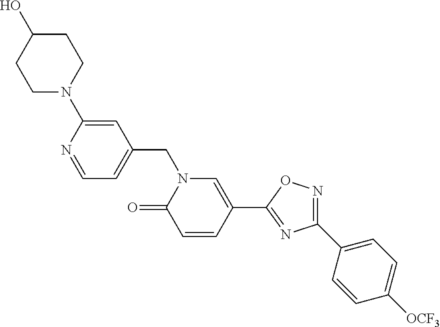 [SMILES: O=c1ccc(-c2nc(-c3ccc(OC(F)(F)F)cc3)no2)cn1Cc1ccnc(N2CCC(O)CC2)c1] | 1-{[2-(4- hydroxypiperidin- 1-yl)pyridin-4-yl] methyl}-5-{3-[4- (trifluoromethoxy) phenyl]-1,2,4- oxadiazol-5-yl}- 1,2-dihydro- pyridin-2-one | 513 | 514 | 7 |
| 164 | 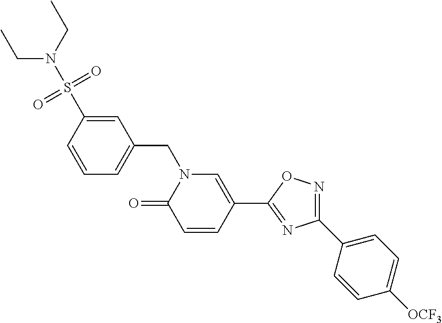 [SMILES: CCN(CC)S(=O)(=O)c1cccc(Cn2cc(-c3nc(-c4ccc(OC(F)(F)F)cc4)no3)ccc2=O)c1] | N,N-diethyl-3- [(2-oxo-5-{3-[4- (trifluoromethoxy) phenyl]-1,2,4- oxadiazol-5-yl}- 1,2-dihydro- pyridin-1-yl) methyl]benzene- 1-sulfonamide | 549 | 550 | 35 |
| 165 | 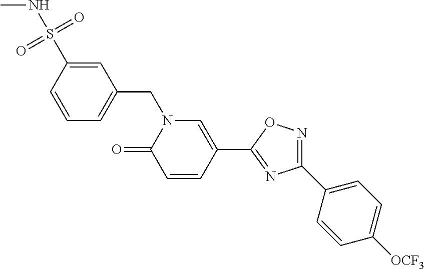 [SMILES: CNS(=O)(=O)c1cccc(Cn2cc(-c3nc(-c4ccc(OC(F)(F)F)cc4)no3)ccc2=O)c1] | N-methyl-3- [(2-oxo-5-{3-[4- (trifluoromethoxy) phenyl]-1,2,4- oxadiazol-5-yl}- 1,2-dihydro- pyridin-1-yl) methyl]benzene- 1-sulfonamide | 506 | 507 | 35 |
| 166 | 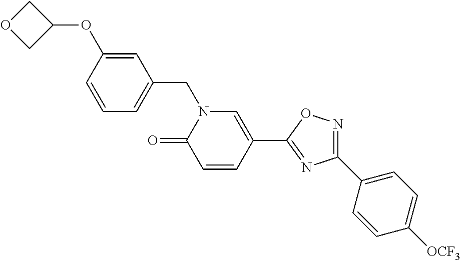 [SMILES: O=c1ccc(-c2nc(-c3ccc(OC(F)(F)F)cc3)no2)cn1Cc1cccc(OC2COC2)c1] | 1-{[3-(oxetan-3- yloxy)phenyl] methyl}-5-{3-[4- (trifluoromethoxy) phenyl]-1,2,4- oxadiazol-5-yl}- 1,2-dihydro- pyridin-2-one | 485 | 486 | 33 |
| 167 | 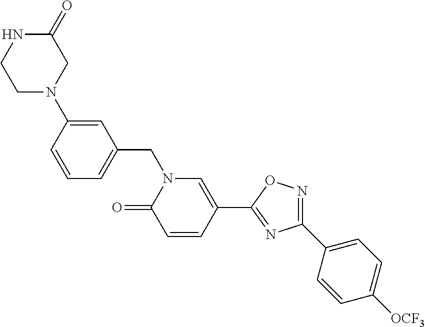 [SMILES: O=C1CN(c2cccc(Cn3cc(-c4nc(-c5ccc(OC(F)(F)F)cc5)no4)ccc3=O)c2)CCN1] | 4-{3-[(2- oxo-5-{3-[4- (trifluoromethoxy) phenyl]-1,2,4- oxadiazol-5-yl}- 1,2-dihydro- pyridin-1-yl) methyl]phenyl} piperazin-2-one | 511 | 512 | 31 |
| 168 | 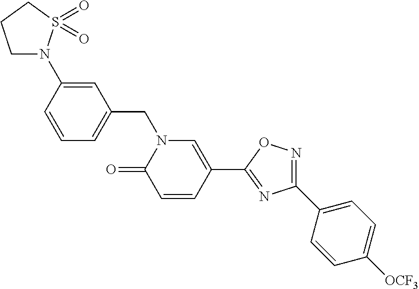 [SMILES: O=c1ccc(-c2nc(-c3ccc(OC(F)(F)F)cc3)no2)cn1Cc1cccc(N2CCCS2(=O)=O)c1] | 1-(3-(1,1-dioxido- isothiazolidin-2- yl)benzyl)-5-(3-(4- (trifluoromethoxy) phenyl)-1,2,4- oxadiazol-5-yl) pyridin-2(1H)-one | 532 | 533 | 31 |
| 169 | 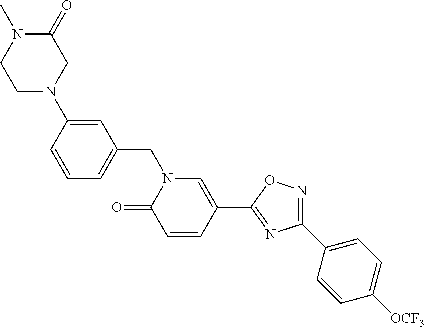 [SMILES: CN1CCN(c2cccc(Cn3cc(-c4nc(-c5ccc(OC(F)(F)F)cc5)no4)ccc3=O)c2)CC1=O] | 1-methyl-4-{3- [(2-oxo-5-{3-[4- (trifluoromethoxy) phenyl]-1,2,4- oxadiazol-5-yl}- 1,2-dihydro- pyridin-1-yl) methyl]phenyl} piperazin-2-one | 525 | 526 | 31 |
| 170 | 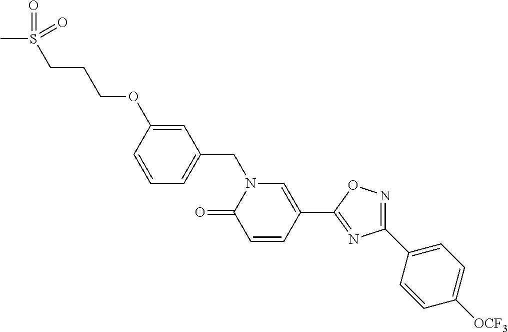 [SMILES: CS(=O)(=O)CCCOc1cccc(Cn2cc(-c3nc(-c4ccc(OC(F)(F)F)cc4)no3)ccc2=O)c1] | 1-{[3-(3- methanesulfonyl- propoxy)phenyl] methyl}-5-{3-[4- (trifluoromethoxy) phenyl]-1,2,4- oxadiazol-5-yl}- 1,2-dihydro- pyridin-2-one | 550 | 551 | 33 |
| 171 | 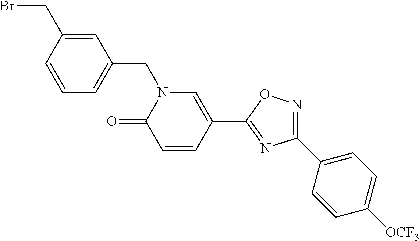 [SMILES: O=c1ccc(-c2nc(-c3ccc(OC(F)(F)F)cc3)no2)cn1Cc1cccc(CBr)c1] | 1-{[3- (bromomethyl) phenyl]methyl}- 5-{3-[4- (trifluoromethoxy) phenyl]-1,2,4- oxadiazol-5-yl}- 1,2-dihydro- pyridin-2-one | 506 | 506; 508 | 26 |
| 172 | 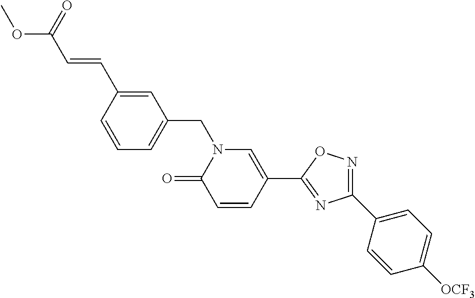 [SMILES: COC(=O)/C=C/c1cccc(Cn2cc(-c3nc(-c4ccc(OC(F)(F)F)cc4)no3)ccc2=O)c1] | methyl (2E)-3-{3- [(2-oxo-5-{3-[4- (trifluoromethoxy) phenyl]-1,2,4- oxadiazol-5-yl}- 1,2-dihydro- pyridin-1-yl) methyl]phenyl} prop-2-enoate | 497 | 498 | 40 |
| 173 | 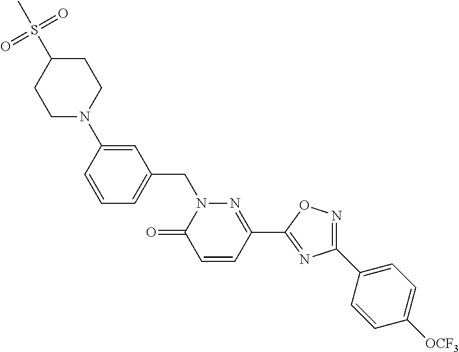 [SMILES: CS(=O)(=O)C1CCN(c2cccc(Cn3nc(-c4nc(-c5ccc(OC(F)(F)F)cc5)no4)ccc3=O)c2)CC1] | 2-{[3-(4- methanesulfonyl- piperidin-1-yl) phenyl]methyl}- 6-{3-[4- (trifluoromethoxy) phenyl]-1,2,4- oxadiazol-5-yl}- 2,3-dihydro- pyridazin-3-one | 576 | 577 | 13 |
| 174 | 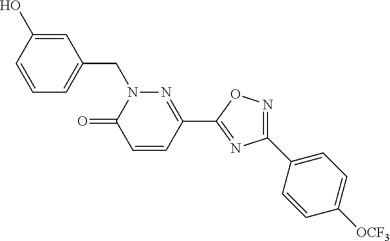 [SMILES: O=c1ccc(-c2nc(-c3ccc(OC(F)(F)F)cc3)no2)nn1Cc1cccc(O)c1] | 2-[(3-hydroxy- phenyl)methyl]- 6-{3-[4- (trifluoromethoxy) phenyl]-1,2,4- oxadiazol-5-yl}- 2,3-dihydro- pyridazin-3-one | 430 | 431 | 24 |
| 175 | 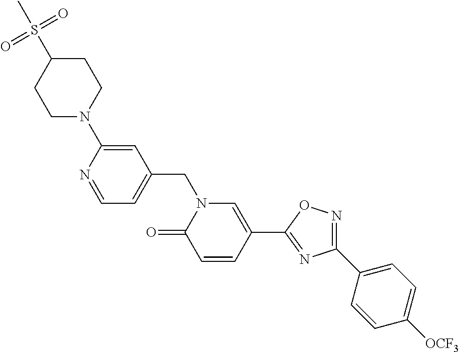 [SMILES: CS(=O)(=O)C1CCN(c2cc(Cn3cc(-c4nc(-c5ccc(OC(F)(F)F)cc5)no4)ccc3=O)ccn2)CC1] | 1-{[2-(4- methanesulfonyl- piperidin-1-yl) pyridin-4-yl] methyl}-5-{3-[4- (trifluoromethoxy) phenyl]-1,2,4- oxadiazol-5-yl}- 1,2-dihydro- pyridin-2-one | 576 | 577 | 7 |
| 176 | 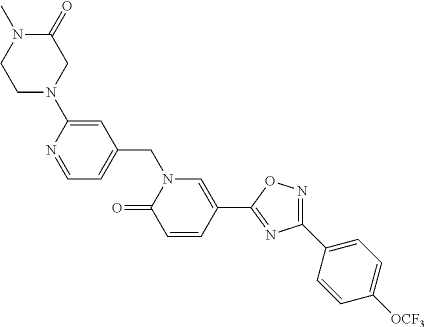 [SMILES: CN1CCN(c2cc(Cn3cc(-c4nc(-c5ccc(OC(F)(F)F)cc5)no4)ccc3=O)ccn2)CC1=O] | 1-methyl-4-{4-[(2- oxo-5-{3-[4- (trifluoromethoxy) phenyl]-1,2,4- oxadiazol-5-yl}- 1,2-dihydro- pyridin-1-yl) methyl]pyridin-2- yl}piperazin-2-one | 526 | 527 | 7 |
| 177 | 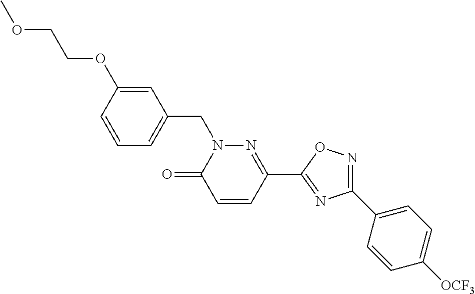 [SMILES: COCCOc1cccc(Cn2nc(-c3nc(-c4ccc(OC(F)(F)F)cc4)no3)ccc2=O)c1] | 2-{[3-(2- methoxyethoxy) phenyl]methyl}- 6-{3-[4- (trifluoromethoxy) phenyl]-1,2,4- oxadiazol-5-yl}- 2,3-dihydro- pyridazin-3-one | 488 | 489 | 33 |
| 178 | 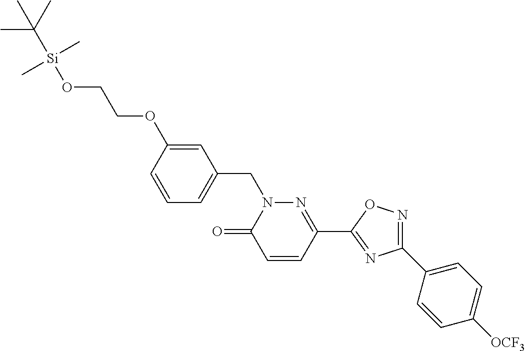 [SMILES: CC(C)(C)[Si](C)(C)OCCOc1cccc(Cn2nc(-c3nc(-c4ccc(OC(F)(F)F)cc4)no3)ccc2=O)c1] | 2-[(3-{2-[(tert- butyldimethyl- silyl)oxy]ethoxy} phenyl)methyl]- 6-{3-[4- (trifluoromethoxy) phenyl]-1,2,4- oxadiazol-5-yl}- 2,3-dihydro- pyridazin-3-one | 589 | 590 | 33 |
| 179 | 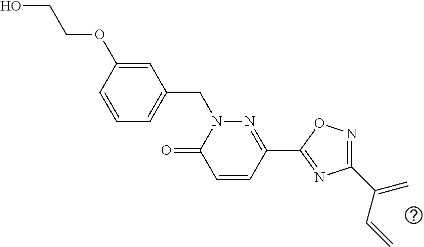 [SMILES: C=CC(=C)c1noc(-c2ccc(=O)n(Cc3cccc(OCCO)c3)n2)n1] | 2-{[3-(2- hydroxyethoxy) phenyl]methyl}- 6-{3-[4- (trifluoromethoxy) phenyl]-1,2,4- oxadiazol-5-yl}- 2,3-dihydro- pyridazin-3-one | 474 | 475 | 33 |
| 180 | 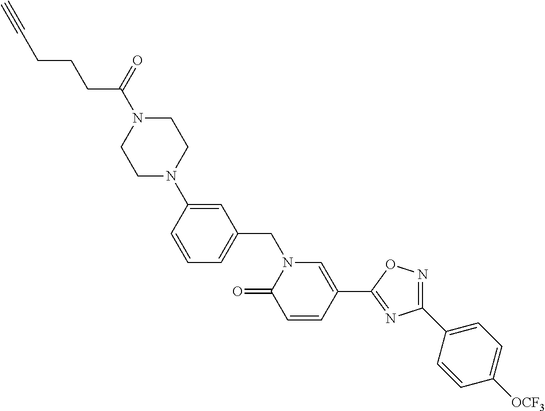 [SMILES: C#CCCCC(=O)N1CCN(c2cccc(Cn3cc(-c4nc(-c5ccc(OC(F)(F)F)cc5)no4)ccc3=O)c2)CC1] | 1-{3-[4-(hex-5- ynoyl)piperazin- 1-yl]phenyl} methyl)-5-{3-[4- (trifluoromethoxy) phenyl]-1,2,4- oxadiazol-5-yl}- 1,2-dihydro- pyridin-2-one | 592 | 593 | 60 |
| 181 | 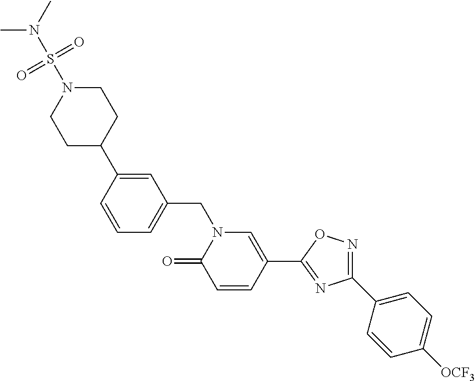 [SMILES: CN(C)S(=O)(=O)N1CCC(c2cccc(Cn3cc(-c4nc(-c5ccc(OC(F)(F)F)cc5)no4)ccc3=O)c2)CC1] | N,N-dimethyl-1- {3-[(2-oxo- 5-{3-[4- (trifluoromethoxy) phenyl]-1,2,4- oxadiazol-5-yl}- 1,2-dihydro- pyridin-1-yl) methyl]phenyl} piperidine-4- sulfonamide | 604 | 605 | 31 |
| 182 | 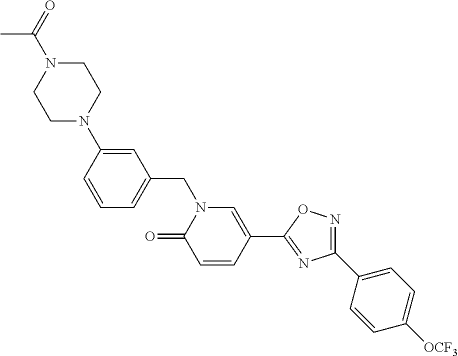 [SMILES: CC(=O)N1CCN(c2cccc(Cn3cc(-c4nc(-c5ccc(OC(F)(F)F)cc5)no4)ccc3=O)c2)CC1] | 1-{[3-(4- acetylpiperazin- 1-yl)phenyl] methyl}-5-{3- (trifluoromethoxy) phenyl]-1,2,4- oxadiazol-5-yl}- 1,2-dihydro- pyridin-2-one | 540 | 541 | 60 |
| 183 | 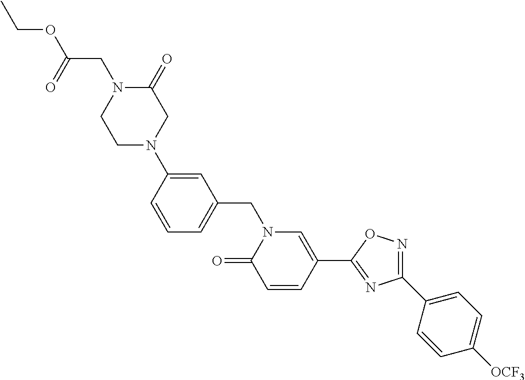 [SMILES: CCOC(=O)CN1CCN(c2cccc(Cn3cc(-c4nc(-c5ccc(OC(F)(F)F)cc5)no4)ccc3=O)c2)CC1=O] | ethyl 2-(2-oxo-4- {3-[(2-oxo- 5-{3-[4- (trifluoromethoxy) phenyl]-1,2,4- oxadiazol-5-yl}- 1,2-dihydro- pyridin-1-yl) methyl]phenyl} piperazin-1- yl)acetate | 598 | 599 | 31 |
| 184 | 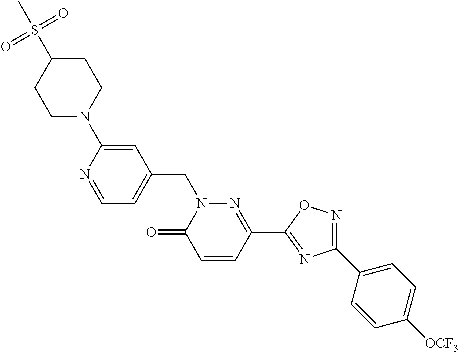 [SMILES: CS(=O)(=O)C1CCN(c2cc(Cn3nc(-c4nc(-c5ccc(OC(F)(F)F)cc5)no4)ccc3=O)ccn2)CC1] | 2-{[2-(4- methanesulfonyl- piperidin-1-yl) pyridin-4-yl] methyl}-6-{3-[4- (trifluoromethoxy) phenyl]-1,2,4- oxadiazol-5-yl}- 2,3-dihydro- pyridazin-3-one | 577 | 578 | 12 |
| 185 | 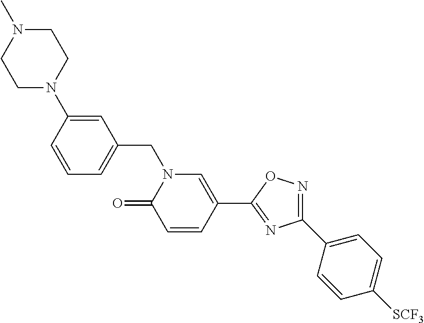 | 1-{[3-(4- methylpiperazin- 1-yl)phenyl] methyl}-5-(3-{4- [(trifluoromethyl) sulfanyl]phenyl}- oxadiazol-5-yl)- 1,2-dihydro- pyridin-2-one | 527 | 528 | 63 |
| 186 | 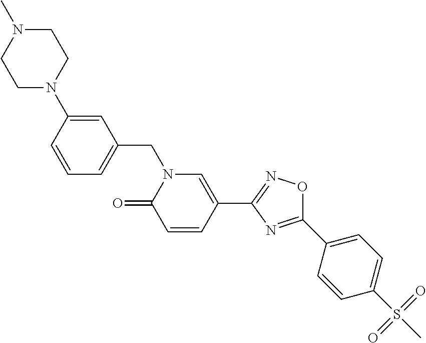 [SMILES: CN1CCN(c2cccc(Cn3cc(-c4noc(-c5ccc(S(C)(=O)=O)cc5)n4)ccc3=O)c2)CC1] | 5-[5-(4- methanesulfonyl- phenyl)-1,2,4- oxadiazol-3- yl]-1-{[3-(4- methylpiperazin- 1-yl)phenyl] methyl}-1,2- dihydropyridin- 2-one | 505 | 506 | 64 |
| 187 | 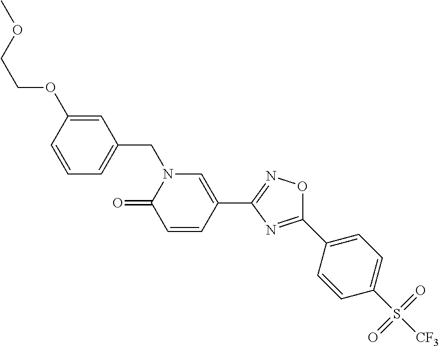 [SMILES: COCCOc1cccc(Cn2cc(-c3noc(-c4ccc(S(=O)(=O)C(F)(F)F)cc4)n3)ccc2=O)c1] | 1-{[3-(2- methoxyethoxy) phenyl]methyl}- 5-[5-(4- trifluoromethane- sulfonylphenyl)- 1,2,4-oxadiazol-3- yl]-1,2-dihydro- pyridin-2-one | 535 | 536 | 65 |
| 188 | 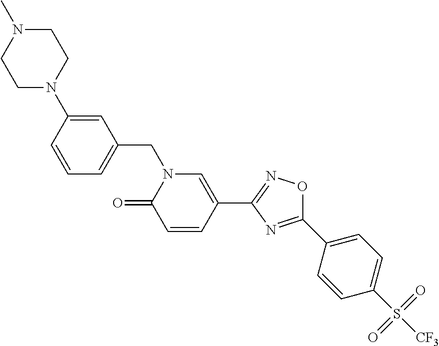 [SMILES: CN1CCN(c2cccc(Cn3cc(-c4noc(-c5ccc(S(=O)(=O)C(F)(F)F)cc5)n4)ccc3=O)c2)CC1] | 1-{[3-(4- methylpiperazin- 1-yl)phenyl] methyl}-5-[5-(4- trifluoromethane- sulfonylphenyl)- 1,2,4-oxadiazol- 3-yl]-1,2-dihydro- pyridin-2-one | 560 | 561 | 64 |
| 189 | 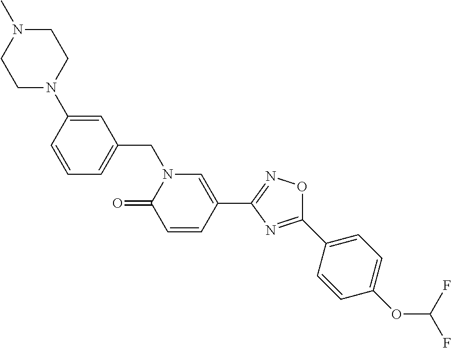 [SMILES: CN1CCN(c2cccc(Cn3cc(-c4noc(-c5ccc(OC(F)F)cc5)n4)ccc3=O)c2)CC1] | 5-{5-[4- (difluoromethoxy) phenyl]-1,2,4- oxadiazol-3- yl}-1-{[3-(4- methylpiperazin- 1-yl)phenyl] methyl}-1,2- dihydropyridin- 2-one | 493 | 494 | 64 |
| 190 | 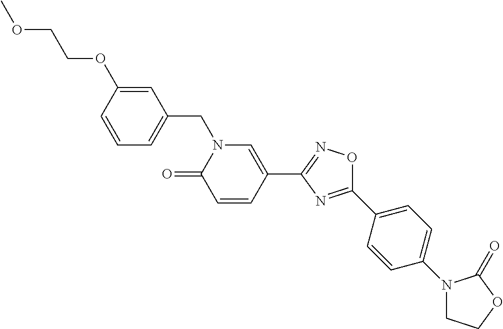 [SMILES: COCCOc1cccc(Cn2cc(-c3noc(-c4ccc(N5CCOC5=O)cc4)n3)ccc2=O)c1] | 1-{[3-(2- methoxyethoxy) phenyl]methyl}- 5-{5-[4-(2-oxo- 1,3-oxazolidin-3- yl)phenyl]-1,2,4- oxadiazol-3-yl}- 1,2-dihydro- pyridin-2-one | 488 | 489 | 66 |
| 191 | 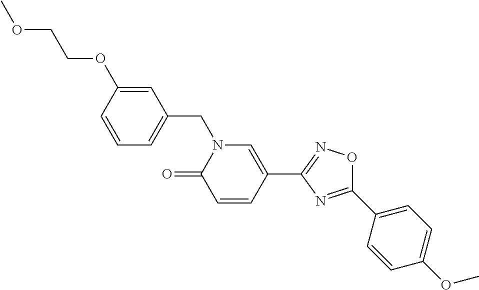 | 1-{[3-(2- methoxyethoxy) phenyl]methyl}- 5-[5-(4- methoxyphenyl)- 1,2,4-oxadiazol-3- yl]-1,2-dihydro- pyridin-2-one | 433 | 434 | 65 |
| 192 | 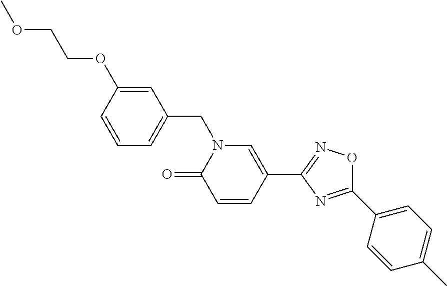 | 1-{[3-(2- methoxyethoxy) phenyl]methyl}- 5-[5-(4- methylphenyl)- 1,2,4-oxadiazol-3- yl]-1,2-dihydro- pyridin-2-one | 417 | 418 | 65 |
| 193 | 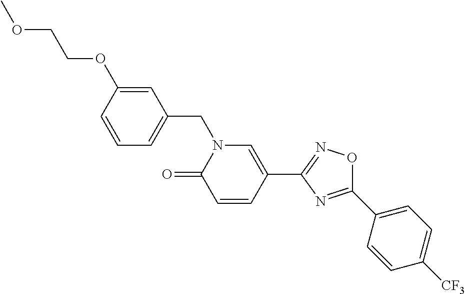 [SMILES: COCCOc1cccc(Cn2cc(-c3noc(-c4ccc(C(F)(F)F)cc4)n3)ccc2=O)c1] | 1-{[3-(2- methoxyethoxy) phenyl]methyl}- 5-{5-[4- (trifluoromethyl) phenyl]-1,2,4- oxadiazol-3-yl}- 1,2-dihydro- pyridin-2-one | 471 | 472 | 65 |
| 194 | 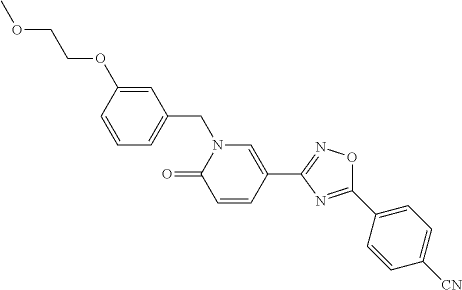 [SMILES: COCCOc1cccc(Cn2cc(-c3noc(-c4ccc(C#N)cc4)n3)ccc2=O)c1] | 4-[3-(1-{[3-(2- methoxyethoxy) phenyl]methyl}-6- oxo-1,6-dihydro- pyridin-3-yl)- 1,2,4-oxadiazol-5- yl]benzonitrile | 428 | 429 | 65 |
| 195 | 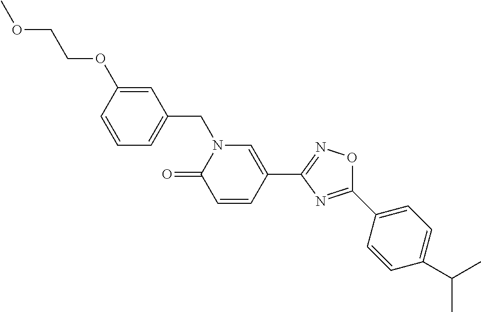 [SMILES: COCCOc1cccc(Cn2cc(-c3noc(-c4ccc(C(C)C)cc4)n3)ccc2=O)c1] | 1-{[3-(2- methoxyethoxy) phenyl]methyl}-5- {5-[4-(propan-2- yl)phenyl]-1,2,4- oxadiazol-3-yl}- 1,2-dihydro- pyridin-2-one | 445 | 446 | 65 |
| 196 | 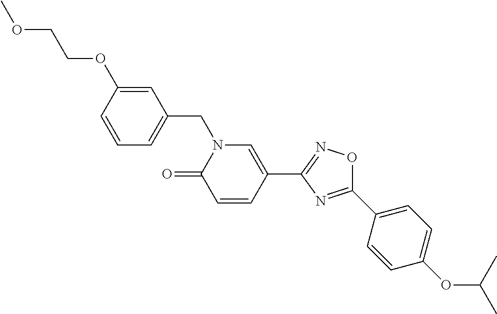 [SMILES: COCCOc1cccc(Cn2cc(-c3noc(-c4ccc(OC(C)C)cc4)n3)ccc2=O)c1] | 1-{[3-(2- methoxyethoxy) phenyl]methyl}- 5-{5-[4-(propan-2- yloxy)phenyl]- 1,2,4-oxadiazol-3- yl}-1,2-dihydro- pyridin-2-one | 461 | 462 | 65 |
| 197 | 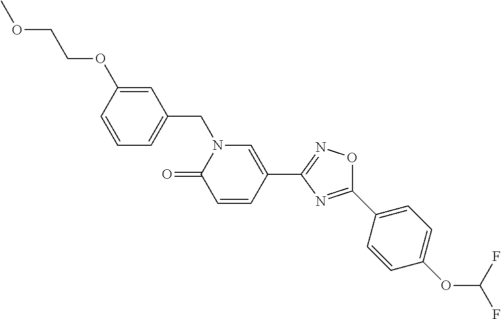 [SMILES: COCCOc1cccc(Cn2cc(-c3noc(-c4ccc(OC(F)F)cc4)n3)ccc2=O)c1] | 5-{5-[4- (difluoromethoxy) phenyl]-1,2,4- oxadiazol-3-yl}- 1-{[3-(2- methoxyethoxy) phenyl]methyl}- 1,2-dihydro- pyridin-2-one | 469 | 470 | 65 |
| 198 | 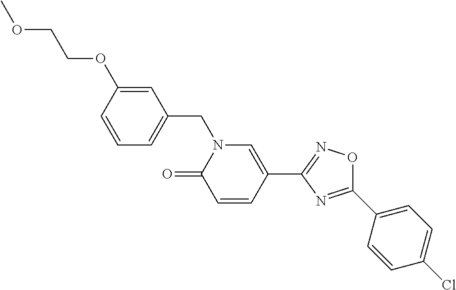 [SMILES: COCCOc1cccc(Cn2cc(-c3noc(-c4ccc(Cl)cc4)n3)ccc2=O)c1] | 5-[5-(4- chlorophenyl)- 1,2,4-oxadiazol-3- yl]-1-{[3-(2- methoxyethoxy) phenyl]methyl}- 1,2-dihydro- pyridin-2-one | 437 | 438 | 65 |
| 199 | 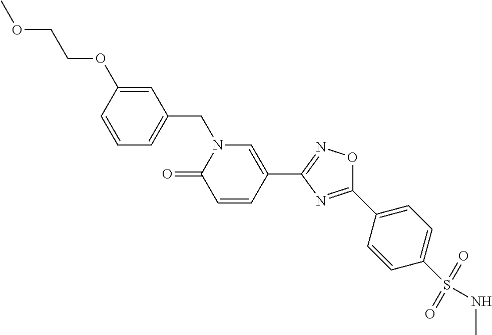 | 4-[3-(1-{[3-(2- methoxyethoxy) phenyl]methyl}-6- oxo-1,6-dihydro- pyridin-3-yl)-1,2, 4-oxadiazol-5-yl]- N-methylbenzene- 1-sulfonamide | 496 | 497 | 65 |
| 200 | 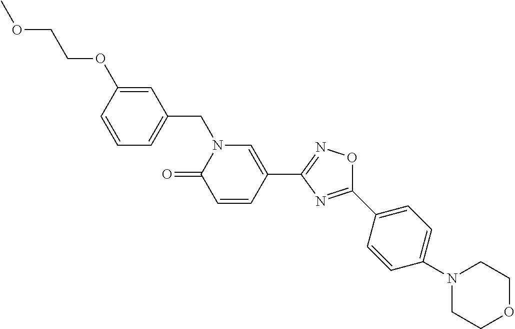 [SMILES: COCCOc1cccc(Cn2cc(-c3noc(-c4ccc(N5CCOCC5)cc4)n3)ccc2=O)c1] | 1-{[3-(2- methoxyethoxy) phenyl]methyl}-5- {5-[4-(morpholin- 4-yl)phenyl]- 1,2,4-oxadiazol-3- yl}-1,2-dihydro- pyridin-2-one | 488 | 489 | 65 |
| 201 | 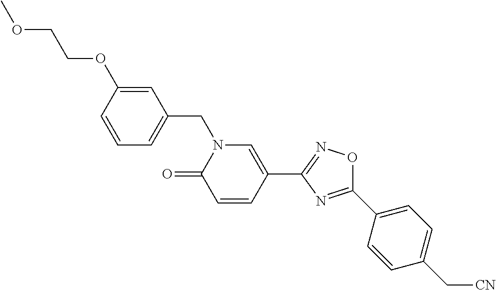 [SMILES: COCCOc1cccc(Cn2cc(-c3noc(-c4ccc(CC#N)cc4)n3)ccc2=O)c1] | 2-{4-[3-(1-{[3-(2- methoxyethoxy) phenyl]methyl}-6- oxo-1,6-dihydro- pyridin-3-yl)- 1,2,4-oxadiazol-5- yl]phenyl} acetonitrile | 442 | 443 | 65 |
| 202 | 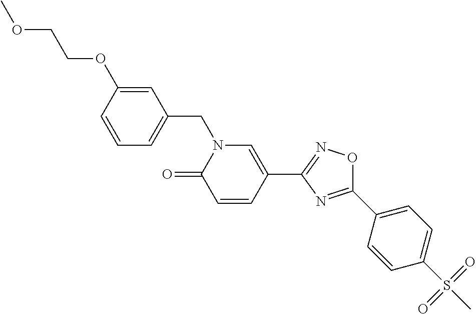 [SMILES: COCCOc1cccc(Cn2cc(-c3noc(-c4ccc(S(C)(=O)=O)cc4)n3)ccc2=O)c1] | 5-[5-(4- methanesulfonyl- phenyl)-1,2,4- oxadiazol-3-yl]- 1-{[3-(2- methoxyethoxy) phenyl]methyl}- 1,2-dihydro- pyridin-2-one | 481 | 482 | 65 |
| 203 | 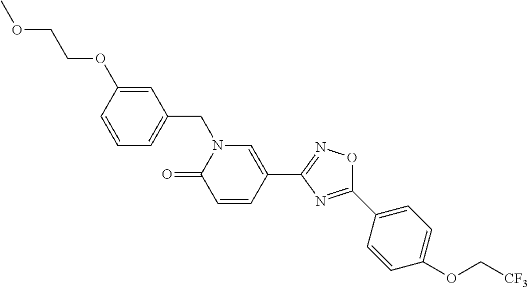 [SMILES: COCCOc1cccc(Cn2cc(-c3noc(-c4ccc(OCC(F)(F)F)cc4)n3)ccc2=O)c1] | 1-{[3-(2- methoxyethoxy) phenyl]methyl}-5- {5-[4-(2,2,2- trifluoroethoxy) phenyl]-1,2,4- oxadiazol-3-yl}- 1,2-dihydro- pyridin-2-one | 501 | 502 | 65 |
| 204 | 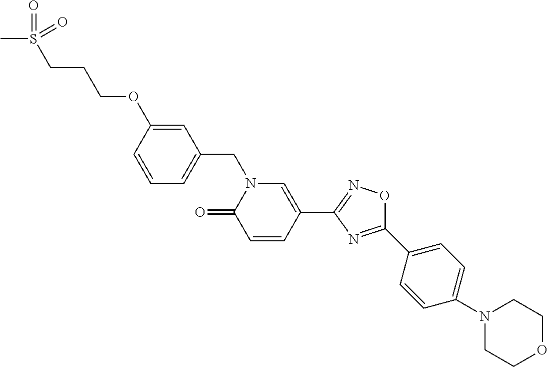 [SMILES: CS(=O)(=O)CCCOc1cccc(Cn2cc(-c3noc(-c4ccc(N5CCOCC5)cc4)n3)ccc2=O)c1] | 1-{[3-(3- methanesulfonyl- propoxy)phenyl] methyl}-5-{5-[4- (morpholin-4- yl)phenyl]-1,2,4- oxadiazol-3-yl}- 1,2-dihydro- pyridin-2-one | 550 | 551 | 69 |
| 205 | 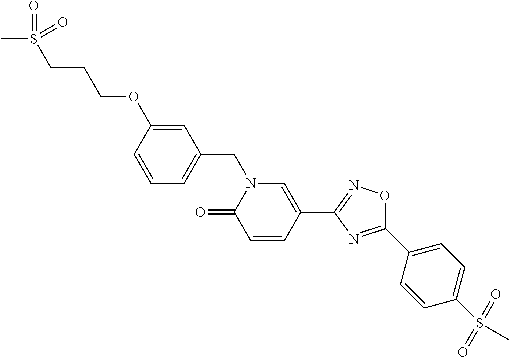 | 5-[5-(4- methanesulfonyl- phenyl)-1,2,4- oxadiazol-3- yl]-1-{[3-(3- methanesulfonyl- propoxy)phenyl] methyl}-1,2- dihydropyridin- 2-one | 543 | 544 | 69 |
| 206 | 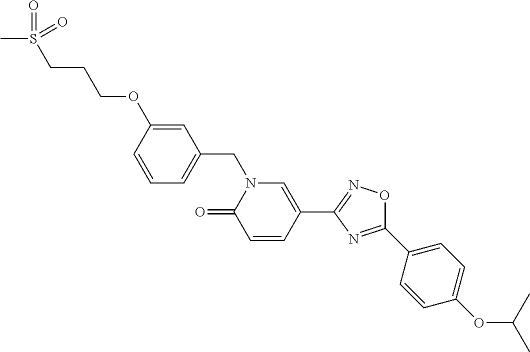 [SMILES: CC(C)Oc1ccc(-c2nc(-c3ccc(=O)n(Cc4cccc(OCCCS(C)(=O)=O)c4)c3)no2)cc1] | 1-{[3-(3- methanesulfonyl- propoxy)phenyl] methyl}-5-{5- [4-(propan-2- yloxy)phenyl]- 1,2,4-oxadiazol-3- yl}-1,2-dihydro- pyridin-2-one | 523 | 524 | 69 |
| 207 | 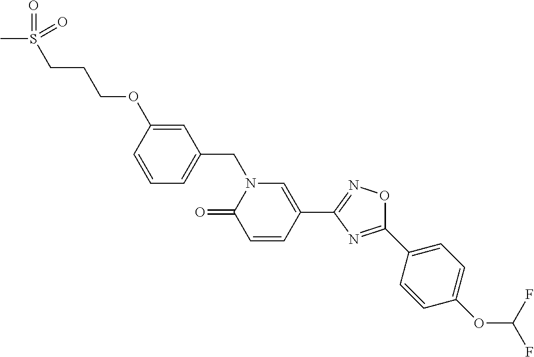 [SMILES: CS(=O)(=O)CCCOc1cccc(Cn2cc(-c3noc(-c4ccc(OC(F)F)cc4)n3)ccc2=O)c1] | 5-{5-[4- (difluoromethoxy) phenyl]-1,2,4- oxadiazol-3- yl}-1-{[3- methanesulfonyl- propoxy)phenyl] methyl}-1,2- dihydropyridin- 2-one | 531 | 532 | 69 |
| 208 | 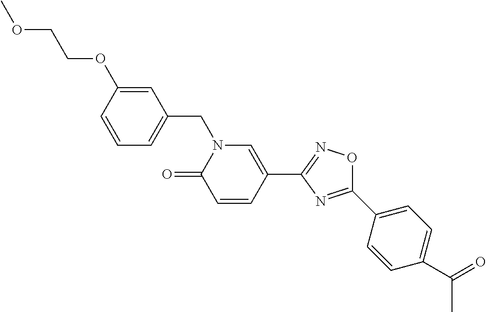 [SMILES: COCCOc1cccc(Cn2cc(-c3noc(-c4ccc(C(C)=O)cc4)n3)ccc2=O)c1] | 5-[5-(4- acetylphenyl)- 1,2,4-oxadiazol-3- yl]-1-{[3-(2- methoxyethoxy) phenyl]methyl}- 1,2-dihydro- pyridin-2-one | 445 | 446 | 65 |
| 209 | 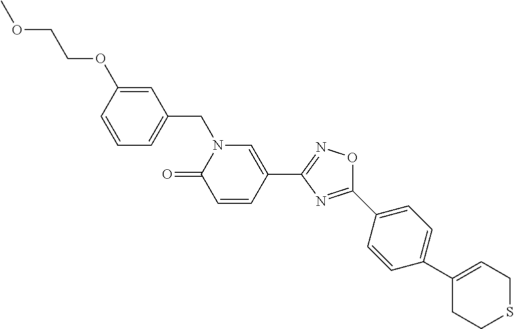 [SMILES: COCCOc1cccc(Cn2cc(-c3noc(-c4ccc(C5=CCSCC5)cc4)n3)ccc2=O)c1] | 5-{5-[4-(3,6- dihydro-2H- thiopyran-4-yl) phenyl]-1,2,4- oxadiazol-3-yl}- 1-{[3-(2- methoxyethoxy) phenyl]methyl}- 1,2-dihydro- pyridin-2-one | 501 | 502 | 74 |
| 210 | 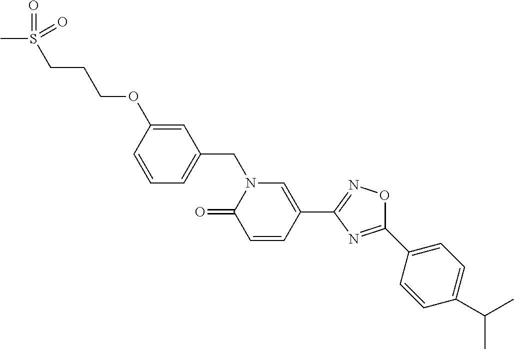 [SMILES: CC(C)c1ccc(-c2nc(-c3ccc(=O)n(Cc4cccc(OCCCS(C)(=O)=O)c4)c3)no2)cc1] | 1-{[3-(3- methanesulfonyl- propoxy)phenyl] methyl}-5-{5- [4-(propan-2- yl)phenyl]-1,2,4- oxadiazol-3-yl}- 1,2-dihydro- pyridin-2-one | 507 | 508 | 69 |
| 211 | 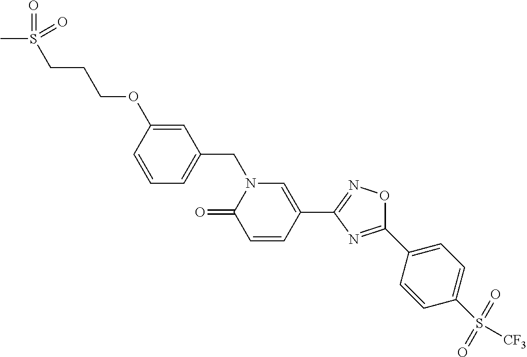 [SMILES: CS(=O)(=O)CCCOc1cccc(Cn2cc(-c3noc(-c4ccc(S(=O)(=O)C(F)(F)F)cc4)n3)ccc2=O)c1] | 1-{[3-(3- methanesulfonyl- propoxy)phenyl] methyl}-5-[5-(4- trifluoromethane- sulfonylphenyl)- 1,2,4-oxadiazol-3- yl]-1,2-dihydro- pyridin-2-one | 597 | 598 | 69 |
| 212 | 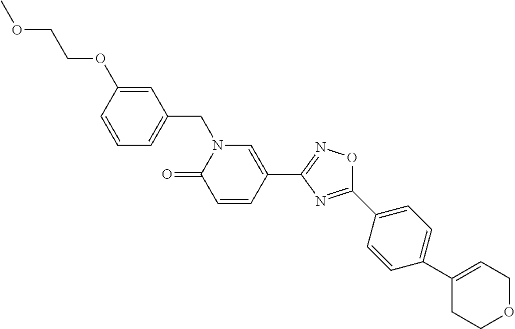 [SMILES: COCCOc1cccc(Cn2cc(-c3noc(-c4ccc(C5=CCOCC5)cc4)n3)ccc2=O)c1] | 5-{5-[4-(3,6- dihydro-2H-pyran- 4-yl)phenyl]- 1,2,4-oxadiazol-3- yl}-1-{[3-(2- methoxyethoxy) phenyl]methyl}- 1,2-dihydro- pyridin-2-one | 485 | 486 | 74 |
| 213 | 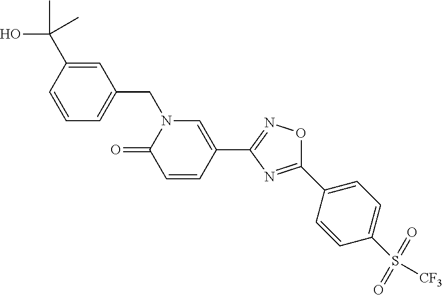 [SMILES: CC(C)(O)c1cccc(Cn2cc(-c3noc(-c4ccc(S(=O)(=O)C(F)(F)F)cc4)n3)ccc2=O)c1] | 1-{[3-(2- hydroxypropan-2- yl)phenyl] methyl}-5-[5-(4- trifluoromethane- sulfonylphenyl)- 1,2,4-oxadiazol-3- yl]-1,2-dihydro- pyridin-2-one | 519 | 520 | 76 |
| 214 | 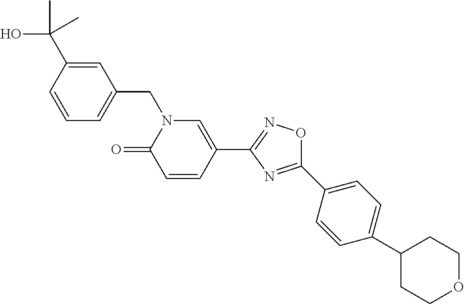 [SMILES: CC(C)(O)c1cccc(Cn2cc(-c3noc(-c4ccc(C5CCOCC5)cc4)n3)ccc2=O)c1] | 1-{[3-(2- hydroxypropan-2- yl)phenyl] methyl}-5-{5- [4-(oxan-4- yl)phenyl]-1,2,4- oxadiazol-3-yl}- 1,2-dihydro- pyridin-2-one | 471 | 472 | 76 |
| 215 | 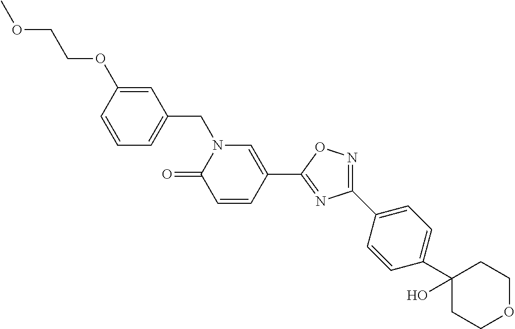 [SMILES: COCCOc1cccc(Cn2cc(-c3nc(-c4ccc(C5(O)CCOCC5)cc4)no3)ccc2=O)c1] | 5-{3-[4-(4- hydroxyoxan-4- yl)phenyl]-1,2,4- oxadiazol-5- yl}-1-{[3-(2- methoxyethoxy) phenyl]methyl}- 1,2-dihydro- pyridin-2-one | 503 | 504 | 80 |
| 216 | 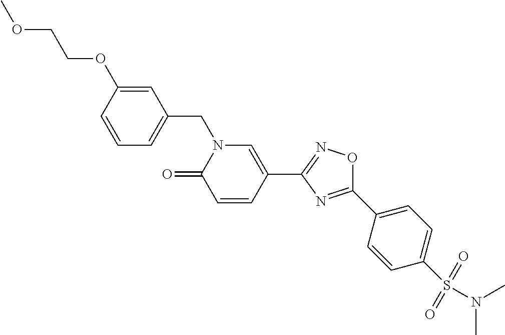 | 4-[3-(1-{[3-(2- methoxyethoxy) phenyl]methyl}-6- oxo-1,6-dihydro- pyridin-3-yl)- 1,2,4-oxadiazol-5- yl]-N,N- dimethylbenzene- 1-sulfonamide | 510 | 511 | 84 |
| 217 | 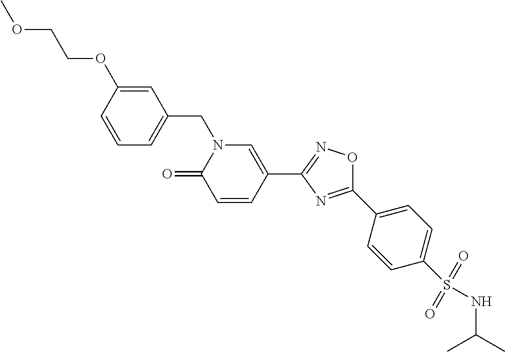 [SMILES: COCCOc1cccc(Cn2cc(-c3noc(-c4ccc(S(=O)(=O)NC(C)C)cc4)n3)ccc2=O)c1] | 4-[3-(1-{[3-(2- methoxyethoxy) phenyl]methyl}-6- oxo-1,6-dihydro- pyridin-3-yl)- 1,2,4-oxadiazol-5- yl}-N-(propan-2- yl)benzene-1- sulfonamide | 524 | 525 | 84 |
| 218 | 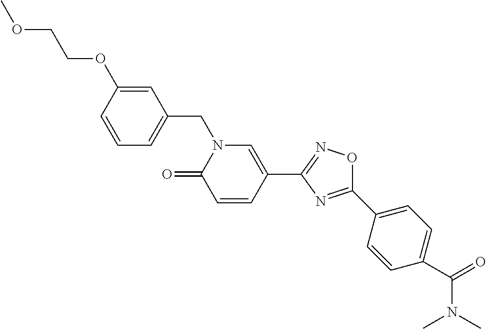 | 4-[3-(1-{[3-(2- methoxyethoxy) phenyl]methyl}-6- oxo-1,6-dihydro- pyridin-3-yl)- 1,2,4-oxadiazol-5- yl]-N,N-dimethyl- benzamide | 474 | 475 | 84 |
| 219 | 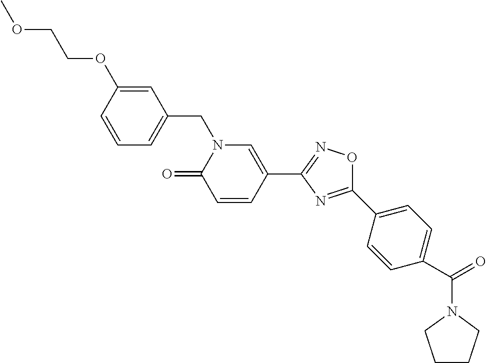 [SMILES: COCCOc1cccc(Cn2cc(-c3noc(-c4ccc(C(=O)N5CCCC5)cc4)n3)ccc2=O)c1] | 1-{[3-(2- methoxyethoxy) phenyl]methyl}-5- {5-[4-(pyrrolidine- 1-carbonyl) phenyl]-1,2,4- oxadiazol-3-yl}- 1,2-dihydro- pyridin-2-one | 500 | 501 | 84 |
| 220 | 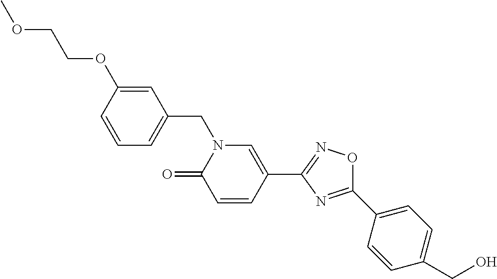 [SMILES: COCCOc1cccc(Cn2cc(-c3noc(-c4ccc(CO)cc4)n3)ccc2=O)c1] | 5-{5-[4- (hydroxymethyl) phenyl]-1,2,4- oxadiazol-3- yl}-1-{[3-(2- methoxyethoxy) phenyl]methyl}- 1,2-dihydro- pyridin-2-one | 433 | 434 | 84 |
| 221 | 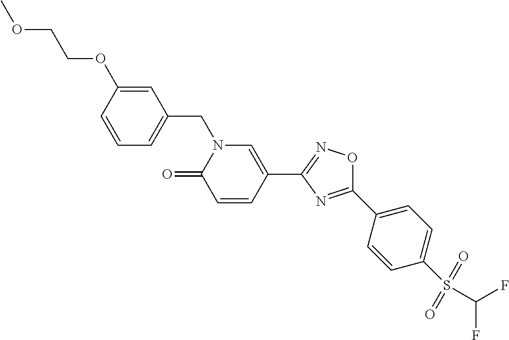 [SMILES: COCCOc1cccc(Cn2cc(-c3noc(-c4ccc(S(=O)(=O)C(F)F)cc4)n3)ccc2=O)c1] | 5-[5-(4- difluoromethane- sulfonylphenyl)- 1,2,4-oxadiazol-3- yl]-1-{[3-(2- methoxyethoxy) phenyl]methyl}- 1,2-dihydro- pyridin-2-one | 517 | 518 | 84 |
| 222 | 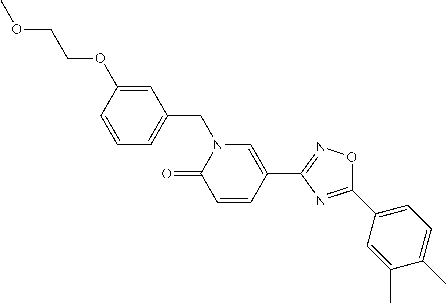 | 5-[5-(3,4- dimethylphenyl)- 1,2,4-oxadiazol-3- yl]-1-{[3-(2- methoxyethoxy) phenyl]methyl}- 1,2-dihydro- pyridin-2-one | 431 | 432 | 84 |
| 223 | 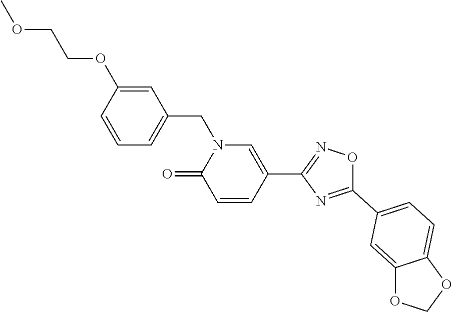 [SMILES: COCCOc1cccc(Cn2cc(-c3noc(-c4ccc5c(c4)OCO5)n3)ccc2=O)c1] | 5-[5-(2H-1,3- benzodioxol-5-yl)- 1,2,4-oxadiazol-3- yl]-1-{[3-(2- methoxyethoxy) phenyl]methyl}- 1,2-dihydro- pyridin-2-one | 447 | 448 | 84 |
| 224 | 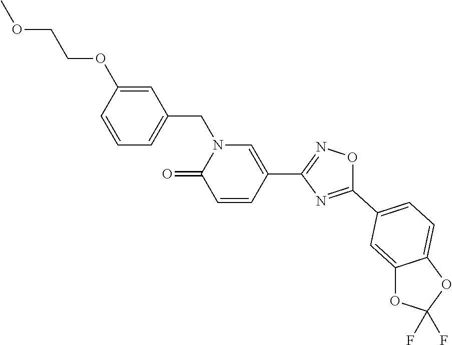 [SMILES: COCCOc1cccc(Cn2cc(-c3noc(-c4ccc5c(c4)OC(F)(F)O5)n3)ccc2=O)c1] | 5-[5-(2,2-difluoro- 2H-1,3- benzodioxol-5-yl)- 1,2,4-oxadiazol-3- yl]-1-{[3-(2- methoxyethoxy) phenyl]methyl}- 1,2-dihydro- pyridin-2-one | 483 | 484 | 84 |
| 225 | 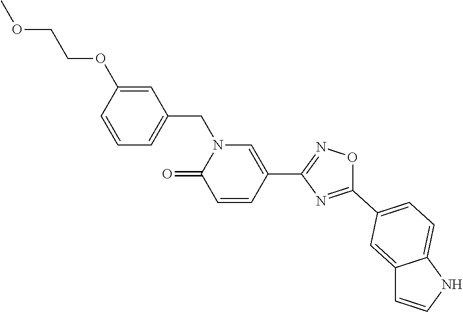 [SMILES: COCCOc1cccc(Cn2cc(-c3noc(-c4ccc5[nH]ccc5c4)n3)ccc2=O)c1] | 5-[5-(1H-indol-5- yl)-1,2,4- oxadiazol-3- yl]-1-{[3-(2- methoxyethoxy) phenyl]methyl}- 1,2-dihydro- pyridin-2-one | 442 | 443 | 84 |
| 226 | 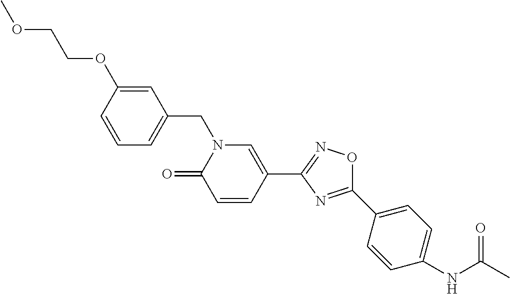 [SMILES: COCCOc1cccc(Cn2cc(-c3noc(-c4ccc(NC(C)=O)cc4)n3)ccc2=O)c1] | N-{4-[3-(1-{[3-(2- methoxyethoxy) phenyl]methyl}-6- oxo-1,6-dihydro- pyridin-3-yl)- 1,2,4-oxadiazol-5- yl]phenyl} acetamide | 460 | 461 | 84 |
| 227 | 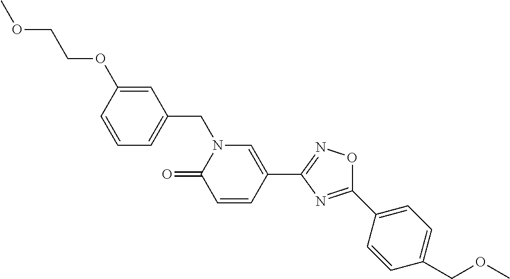 | 1-{[3-(2- methoxyethoxy) phenyl]methyl}-5- {5-[4- (methoxymethyl) phenyl]-1,2,4- oxadiazol-3-yl}- 1,2-dihydro- pyridin-2-one | 447 | 448 | 84 |
| 228 | 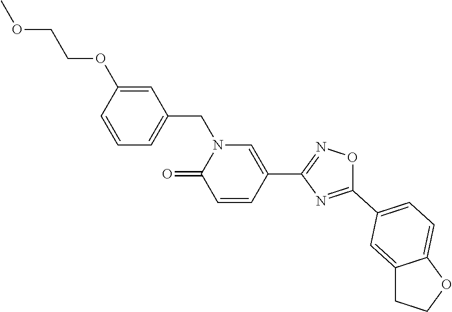 [SMILES: COCCOc1cccc(Cn2cc(-c3noc(-c4ccc5c(c4)CCO5)n3)ccc2=O)c1] | 5-[5-(2,3-dihydro- 1-benzofuran- 5-yl)-1,2,4- oxadiazol-3- yl]-1-{[3-(2- methoxyethoxy) phenyl]methyl}- 1,2-dihydro- pyridin-2-one | 445 | 446 | 84 |
| 229 | 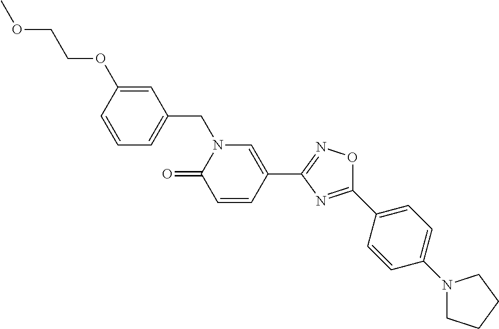 [SMILES: COCCOc1cccc(Cn2cc(-c3noc(-c4ccc(N5CCCC5)cc4)n3)ccc2=O)c1] | 1-{[3-(2- methoxyethoxy) phenyl]methyl}-5- {5-[4-(pyrrolidin- 1-yl)phenyl]- 1,2,4-oxadiazol-3- yl}-1,2-dihydro- pyridin-2-one | 472 | 473 | 84 |
| 230 | 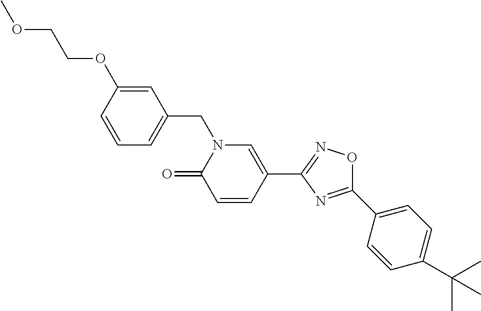 [SMILES: COCCOc1cccc(Cn2cc(-c3noc(-c4ccc(C(C)(C)C)cc4)n3)ccc2=O)c1] | 5-[5-(4-tert- butylphenyl)- 1,2,4-oxadiazol-3- yl]-1-{[3-(2- methoxyethoxy) phenyl]methyl}- 1,2-dihydro- pyridin-2-one | 459 | 460 | 84 |
| 231 | 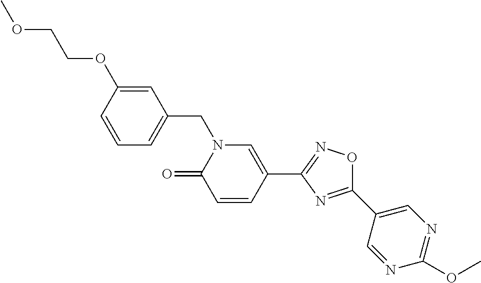 | 1-{[3-(2- methoxyethoxy) phenyl]methyl}-5- [5-(2-methoxy- pyrimidin-5-yl)- 1,2,4-oxadiazol-3- yl]-1,2-dihydro- pyridin-2-one | 435 | 436 | 87 |
| 232 | 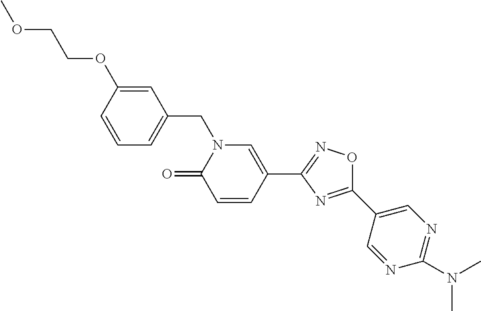 | 5-{5-[2- (dimethylamino) pyrimidin-5-yl]- 1,2,4-oxadiazol-3- yl}-1-{[3-(2- methoxyethoxy) phenyl]methyl}- 1,2-dihydro- pyridin-2-one | 448 | 449 | 88 |
| 233 | 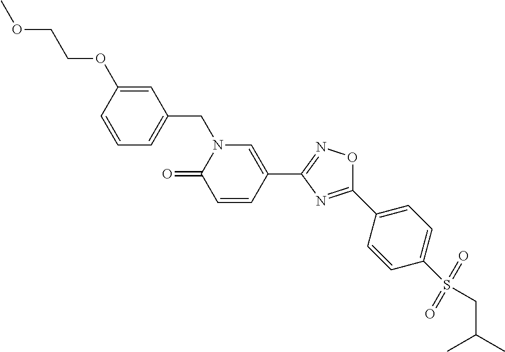 [SMILES: COCCOc1cccc(Cn2cc(-c3noc(-c4ccc(S(=O)(=O)CC(C)C)cc4)n3)ccc2=O)c1] | 1-{[3-(2- methoxyethoxy) phenyl]methyl}-5- {5-[4-(2- methylpropane- sulfonyl)phenyl]- 1,2,4-oxadiazol-3- yl}-1,2-dihydro- pyridin-2-one | 523 | 524 | 84 |
| 234 | 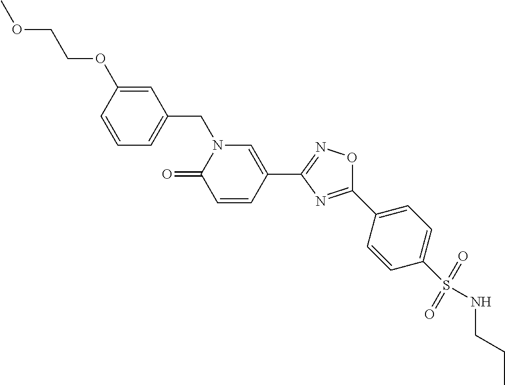 [SMILES: CCCNS(=O)(=O)c1ccc(-c2nc(-c3ccc(=O)n(Cc4cccc(OCCOC)c4)c3)no2)cc1] | 4-[3-(1-{[3-(2- methoxyethoxy) phenyl]methyl}-6- oxo-1,6- dihydroylpyridin- 3-yl)-1,2,4- oxadiazol-5-yl]-N- propylbenzene-1- sulfonamide | 524 | 525 | 84 |
| 235 | 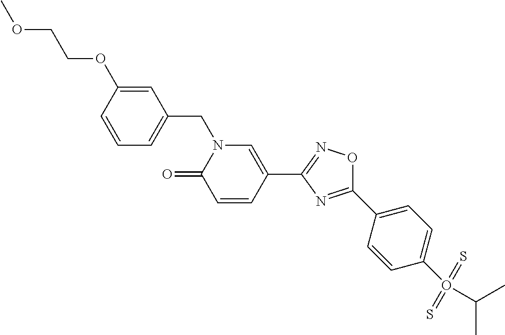 | 1-{[3-(2- methoxyethoxy) phenyl]methyl}-5- {5-[4-(propane-2- sulfonyl)phenyl]- 1,2,4-oxadiazol-3- yl}-1,2-dihydro- pyridin-2-one | 509 | 510 | 84 |
| 236 | 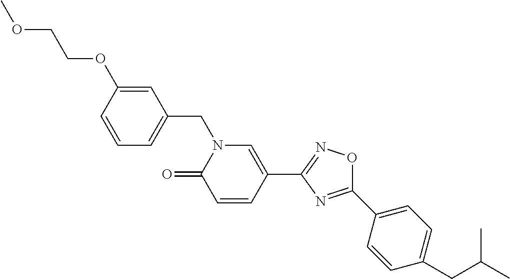 [SMILES: COCCOc1cccc(Cn2cc(-c3noc(-c4ccc(CC(C)C)cc4)n3)ccc2=O)c1] | 1-{[3-(2- methoxyethoxy) phenyl]methyl}-5- {5-[4-(2- methylpropyl) phenyl]-1,2,4- oxadiazol-3-yl}- 1,2-dihydro- pyridin-2-one | 459 | 460 | 84 |
| 237 | 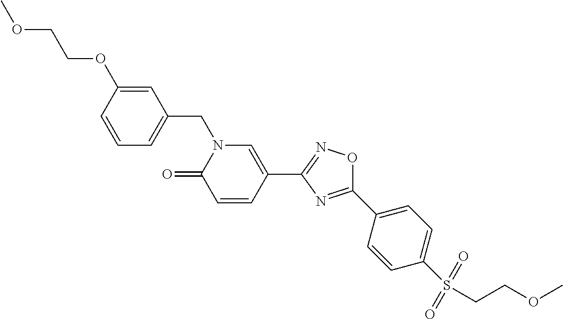 | 5-{5-[4-(2- methoxyethane- sulfonyl)phenyl]- 1,2,4-oxadiazol-3- yl}-1-{[3-(2- methoxyethoxy) phenyl]methyl}- 1,2-dihydro- pyridin-2-one | 525 | 526 | 84 |
| 238 | 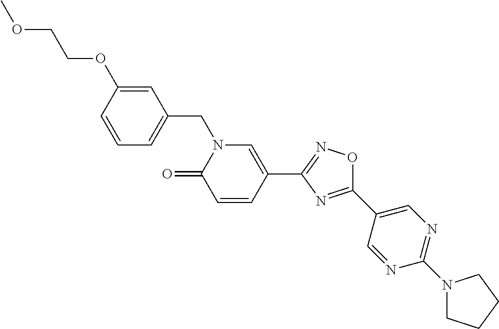 [SMILES: COCCOc1cccc(Cn2cc(-c3noc(-c4cnc(N5CCCC5)nc4)n3)ccc2=O)c1] | 1-{[3-(2- methoxyethoxy) phenyl]methyl}-5- {5-[2-(pyrrolidin- 1-yl)pyrimidin- 5-yl]-1,2,4- oxadiazol-3-yl}- 1,2-dihydro- pyridin-2-one | 474 | 475 | 88 |
| 239 | 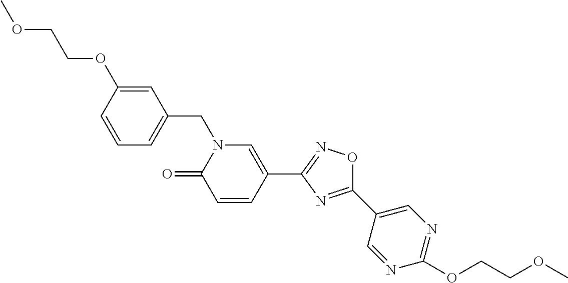 | 1-{[3-(2- methoxyethoxy) phenyl]methyl}-5- {5-[2-(2- methoxyethoxy) pyrimidin-5-yl]- 1,2,4-oxadiazol-3- yl}-1,2-dihydro- pyridin-2-one | 479 | 480 | 87 |
| 240 | 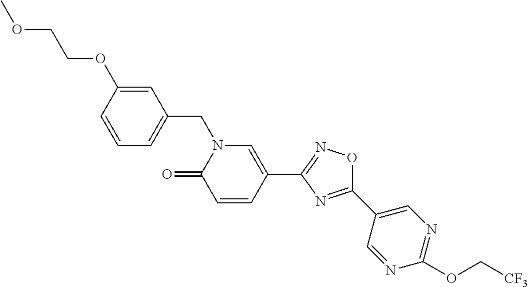 [SMILES: COCCOc1cccc(Cn2cc(-c3noc(-c4cnc(OCC(F)(F)F)nc4)n3)ccc2=O)c1] | 1-{[3-(2- methoxyethoxy) phenyl]methyl}-5- {5-[2-(2,2,2- trifluoroethoxy) pyrimidin-5-yl]- 1,2,4-oxadiazol-3- yl}-1,2-dihydro- pyridin-2-one | 503 | 504 | 87 |
| 241 | 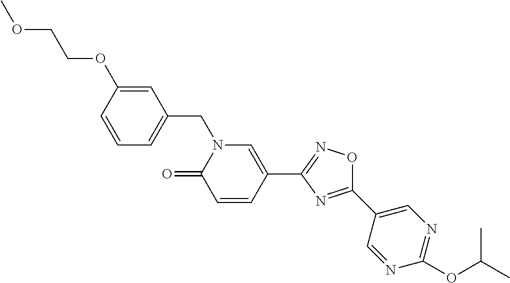 [SMILES: COCCOc1cccc(Cn2cc(-c3noc(-c4cnc(OC(C)C)nc4)n3)ccc2=O)c1] | 1-{[3-(2- methoxyethoxy) phenyl]methyl}-5- {5-[2-(propan-2- yloxy)pyrimidin- 5-yl]-1,2,4- oxadiazol-3-yl}-1, 2-dihydropyridin- 2-one | 463 | 464 | 87 |
| 242 | 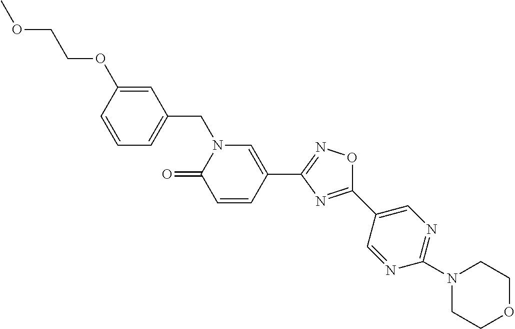 [SMILES: COCCOc1cccc(Cn2cc(-c3noc(-c4cnc(N5CCOCC5)nc4)n3)ccc2=O)c1] | 1-{[3-(2- methoxyethoxy) phenyl]methyl}-5- {5-[2-(morpholin- 4-yl)pyrimidin- 5-yl]-1,2,4- oxadiazol-3-yl}- 1,2-dihydro- pyridin-2-one | 490 | 491 | 88 |
| 243 | 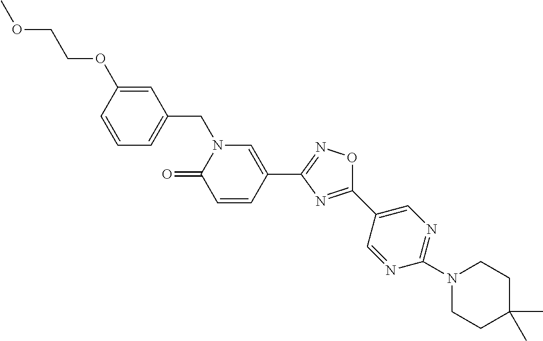 | 5-{5-[2-(4,4- dimethylpiperidin- 1-yl)pyrimidin- 5-yl]-1,2,4- oxadiazol-3- yl}-1-{[3-(2- methoxyethoxy) phenyl]methyl}- 1,2-dihydro- pyridin-2-one | 516 | 517 | 88 |
| 244 | 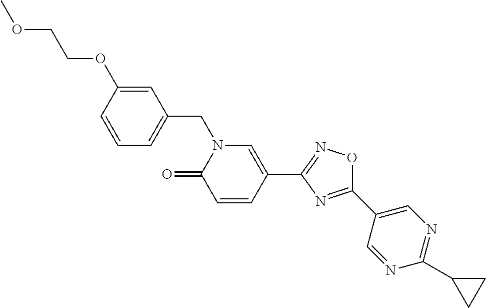 [SMILES: COCCOc1cccc(Cn2cc(-c3noc(-c4cnc(C5CC5)nc4)n3)ccc2=O)c1] | 5-[5-(2-cyclo- propylpyrimidin- 5-yl)-1,2,4- oxadiazol-3- yl]-1-{[3-(2- methoxyethoxy) phenyl]methyl} 1,2-dihydro- pyridin-2-one | 445 | 446 | 84 |
| 245 | 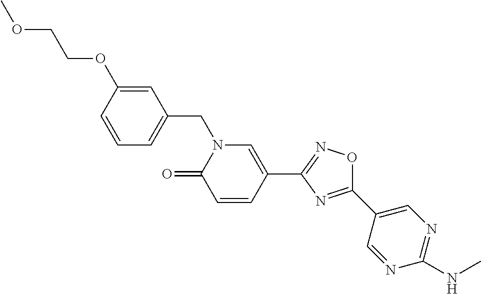 | 1-{[3-(2- methoxyethoxy) phenyl]methyl}- 5-{5-[2- (methylamino) pyrimidin-5-yl]- 1,2,4-oxadiazol-3- yl}-1,2-dihydro- pyridin-2-one | 434 | 435 | 88 |
| 246 | 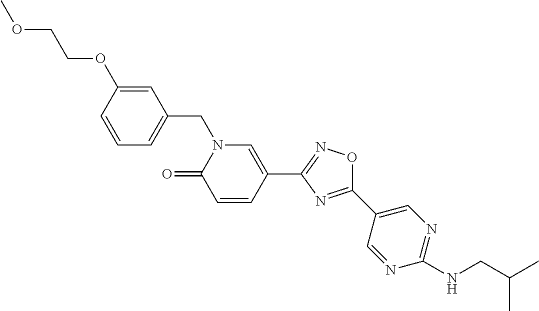 [SMILES: COCCOc1cccc(Cn2cc(-c3noc(-c4cnc(NCC(C)C)nc4)n3)ccc2=O)c1] | 1-{[3-(2- methoxyethoxy) phenyl]methyl}-5- (5-{2-[2- methylpropyl) amino]pyrimidin- 5-yl}-1,2,4- oxadiazol-3- yl)-1,2-dihydro- pyridin-2-one | 476 | 477 | 88 |
| 247 | 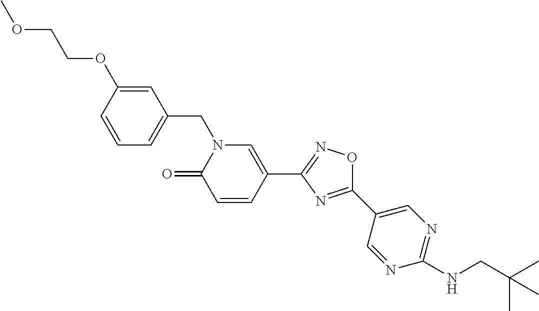 [SMILES: COCCOc1cccc(Cn2cc(-c3noc(-c4cnc(NCC(C)(C)C)nc4)n3)ccc2=O)c1] | 5-(5-{2-[(2,2- dimethylpropyl) amino]pyrimidin- 5-yl}-1,2,4- oxadiazol-3- yl)-1-{[3-(2- methoxyethoxy) phenyl]methyl}- 1,2-dihydro- pyridin-2-one | 490 | 491 | 88 |
| 248 | 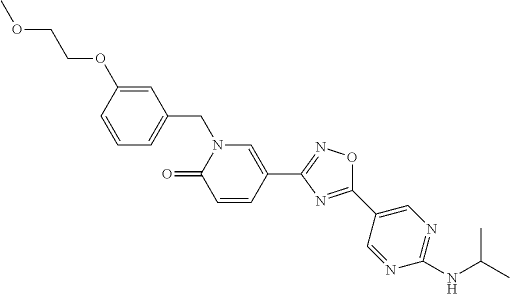 [SMILES: COCCOc1cccc(Cn2cc(-c3noc(-c4cnc(NC(C)C)nc4)n3)ccc2=O)c1] | 1-{[3-(2- methoxyethoxy) phenyl]methyl}-5- (5-{2-[(propan-2- yl)amino] pyrimidin-5-yl}- 1,2,4-oxadiazol-3- yl)-1,2-dihydro- pyridin-2-one | 462 | 463 | 88 |
| 249 | 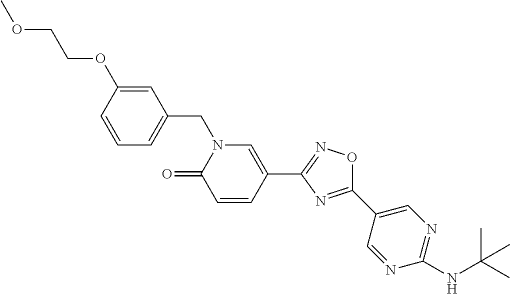 [SMILES: COCCOc1cccc(Cn2cc(-c3noc(-c4cnc(NC(C)(C)C)nc4)n3)ccc2=O)c1] | 5-{5-[2-(tert- butylamino) pyrimidin-5-yl]- 1,2,4-oxadiazol-3- yl}-1-{[3-(2- methoxyethoxy) phenyl]methyl}- 1,2-dihydro- pyridin-2-one | 476 | 477 | 88 |
| 250 | 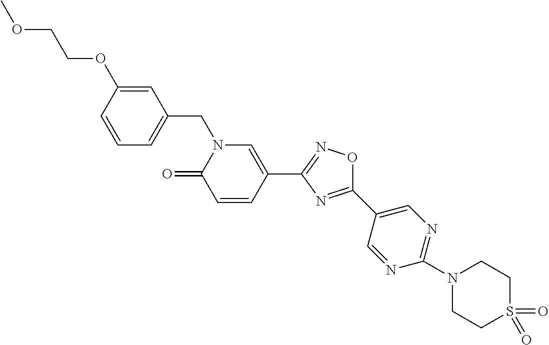 [SMILES: COCCOc1cccc(Cn2cc(-c3noc(-c4cnc(N5CCS(=O)(=O)CC5)nc4)n3)ccc2=O)c1] | 5-(5-(2-(1,1- dioxidothio morpholino) pyrimidin- 5-yl)-1,2,4- oxadiazol-3- yl)-1-(3-(2- methoxyethoxy) benzyl)pyridin- 2(1H)-one | 538 | 539 | 88 |
| 251 | 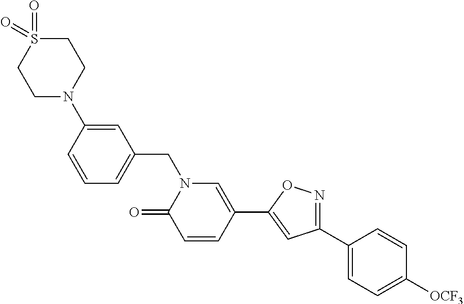 [SMILES: O=c1ccc(-c2cc(-c3ccc(OC(F)(F)F)cc3)no2)cn1Cc1cccc(N2CCS(=O)(=O)CC2)c1] | 1-(3-(1,1- dioxidothio morpholino) benzyl)-5-(3-(4- (trifluoromethoxy) phenyl)isoxazol-5- yl)pyridin-2(1H)- one | 545 | 546 | 98 |
| 252 | 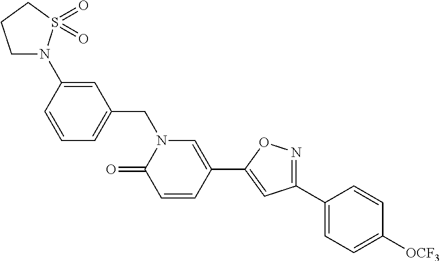 [SMILES: O=c1ccc(-c2cc(-c3ccc(OC(F)(F)F)cc3)no2)cn1Cc1cccc(N2CCCS2(=O)=O)c1] | 1-(3-(1,1- dioxidoiso- thiazolidin-2-yl) benzyl)-5-(3-(4- (trifluoromethoxy) phenyl)isoxazol- 5-yl)pyridin-2(1H)-one | 531 | 532 | 98 |
| 253 | 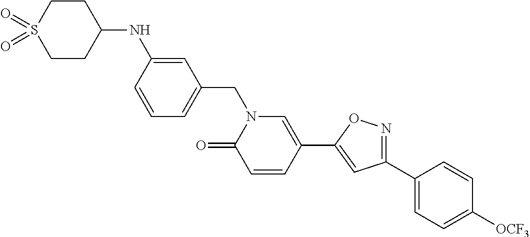 [SMILES: O=c1ccc(-c2cc(-c3ccc(OC(F)(F)F)cc3)no2)cn1Cc1cccc(NC2CCS(=O)(=O)CC2)c1] | 1-(3-((1,1- dioxidotetrahydro- 2H-thiopyran-4- yl)amino)benzyl)- 5-(3-(4- (trifluoromethoxy) phenyl)isoxazol- 5-yl)pyridin- 2(1H)-one | 559 | 560 | 98 |
| 254 | 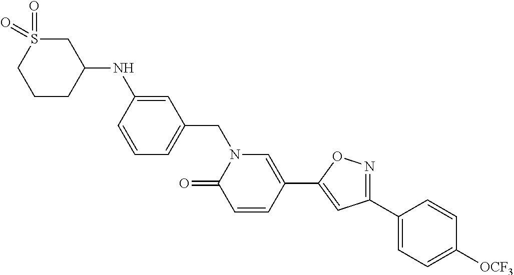 [SMILES: O=c1ccc(-c2cc(-c3ccc(OC(F)(F)F)cc3)no2)cn1Cc1cccc(NC2CCCS(=O)(=O)C2)c1] | 1-(3-((1,1- dioxidotetrahydro- 2H-thiopyran-3- yl)amino)benzyl)- 5-(3-(4- (trifluoromethoxy) phenyl)isoxazol- 5-yl)pyridin- 2(1H)-one | 559 | 560 | 98 |
| 255 | 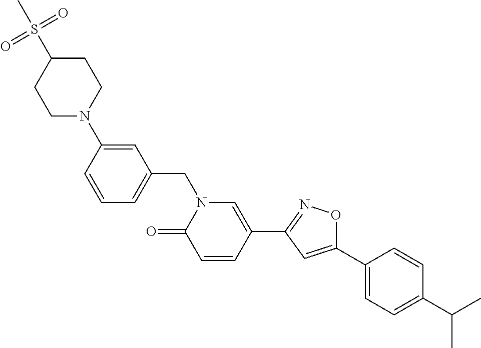 [SMILES: CC(C)c1ccc(-c2cc(-c3ccc(=O)n(Cc4cccc(N5CCC(S(C)(=O)=O)CC5)c4)c3)no2)cc1] | 5-(5-(4- isopropylphenyl) isoxazol-3-yl)-1- (3-(4- (methylsulfonyl) piperidin-1-yl) benzyl)pyridin- 2(1H)-one | 531 | 532 | 99 |
| 257 | 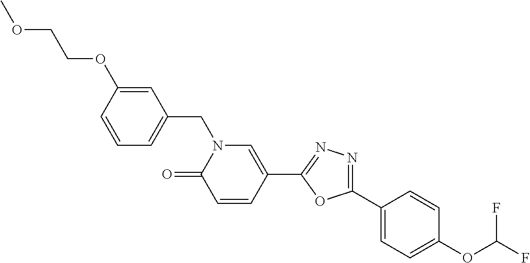 [SMILES: COCCOc1cccc(Cn2cc(-c3nnc(-c4ccc(OC(F)F)cc4)o3)ccc2=O)c1] | 5-{5-[4- (difluoromethoxy) phenyl]-1,3,4- oxadiazol-2- yl}-1-{[3-(2- methoxyethoxy) phenyl]methyl}- 1,2-dihydro- pyridin-2-one | 469 | 470 | 102 |
| 258 | 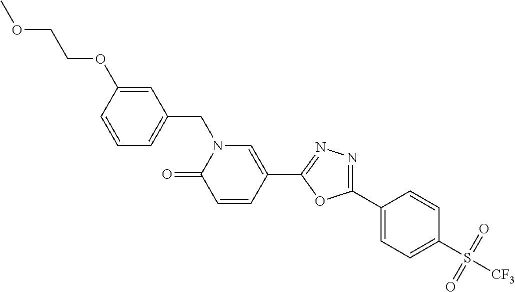 [SMILES: COCCOc1cccc(Cn2cc(-c3nnc(-c4ccc(S(=O)(=O)C(F)(F)F)cc4)o3)ccc2=O)c1] | 1-{[3-(2- methoxyethoxy) phenyl]methyl}-5- [5-(4-trifluoro- methanesulfonyl- phenyl)-1,3,4- oxadiazol-2-yl]- 1,2-dihydro- pyridin-2-one | 534 | 535 | 102 |
| 259 | 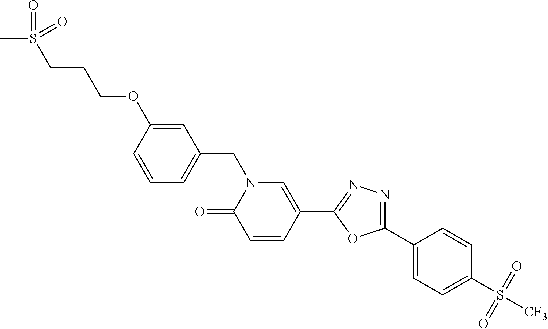 [SMILES: CS(=O)(=O)CCCOc1cccc(Cn2cc(-c3nnc(-c4ccc(S(=O)(=O)C(F)(F)F)cc4)o3)ccc2=O)c1] | 1-{[3-(3- methanesulfonyl- propoxy)phenyl] methyl}-5-[5-(4- trifluoromethane- sulfonylphenyl)- 1,3,4-oxadiazol-2- yl]-1,2-dihydro- pyridin-2-one | 597 | 598 | 105 |
| 260 | 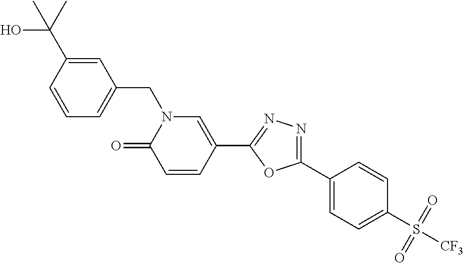 [SMILES: CC(C)(O)c1cccc(Cn2cc(-c3nnc(-c4ccc(S(=O)(=O)C(F)(F)F)cc4)o3)ccc2=O)c1] | 1-{[3-(2- hydroxypropan-2- yl)phenyl] methyl}-5-[5-(4- trifluoromethane- sulfonylphenyl)- 1,3,4-oxadiazol-2- yl]-1,2-dihydro- pyridin-2-one | 519 | 520 | 103 |
| 261 | 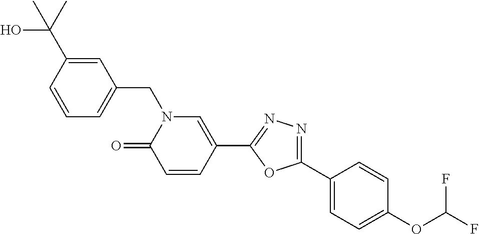 [SMILES: CC(C)(O)c1cccc(Cn2cc(-c3nnc(-c4ccc(OC(F)F)cc4)o3)ccc2=O)c1] | 5-{5-[4- (difluoromethoxy) phenyl]-1,3,4- oxadiazol-2- yl}-1-{[3-(2- hydroxypropan-2- yl)phenyl] methyl}-1,2- dihydropyridin- 2-one | 453 | 454 | 103 |
| 262 | 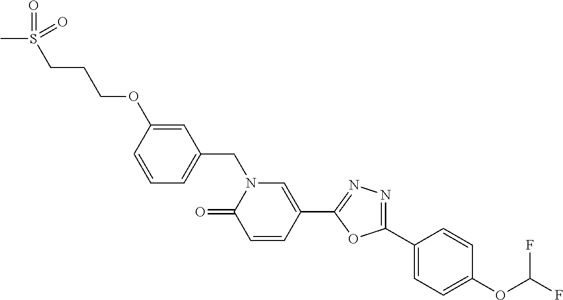 [SMILES: CS(=O)(=O)CCCOc1cccc(Cn2cc(-c3nnc(-c4ccc(OC(F)F)cc4)o3)ccc2=O)c1] | 5-{5-[4- (difluoromethoxy) phenyl]-1,3,4- oxadiazol-2- yl}-1-{[3-(3- methanesulfonyl- propoxy)phenyl] methyl}-1,2- dihydropyridin- 2-one | 532 | 533 | 105 |
| 263 | 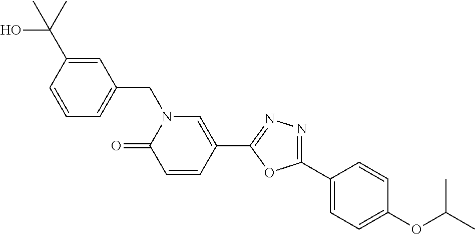 [SMILES: CC(C)Oc1ccc(-c2nnc(-c3ccc(=O)n(Cc4cccc(C(C)(C)O)c4)c3)o2)cc1] | 1-{[3-(2- hydroxypropan-2- yl)phenyl] methyl}-5-{5- [4-(propan-2- yloxy)phenyl]- 1,3,4-oxadiazol-2- yl}-1,2-dihydro- pyridin-2-one | 445 | 446 | 103 |
| 264 | 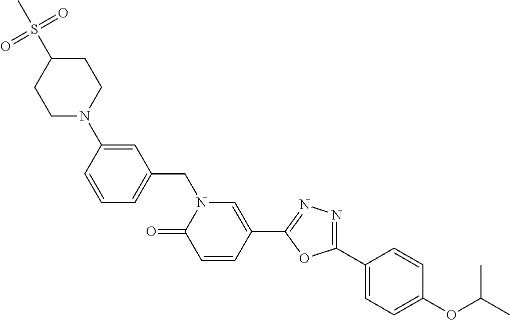 [SMILES: CC(C)Oc1ccc(-c2nnc(-c3ccc(=O)n(Cc4cccc(N5CCC(S(C)(=O)=O)CC5)c4)c3)o2)cc1] | 1-{[3-[4-methane- sulfonylpiperidin- 1-yl)phenyl] methyl}-5-{5- [4-(propan-2- yloxy)phenyl]- 1,3,4-oxadiazol-2- yl}-1,2-dihydro- pyridin-2-one | 548 | 549 | 104 |
| 265 | 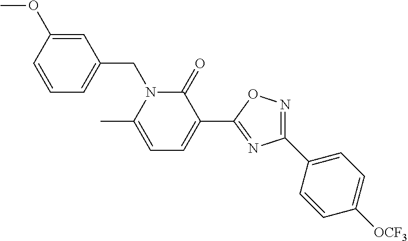 [SMILES: COc1cccc(Cn2c(C)ccc(-c3nc(-c4ccc(OC(F)(F)F)cc4)no3)c2=O)c1] | 1-[(3- methoxyphenyl) methyl]-6-methyl- 3-{3-[4- (trifluoromethoxy) phenyl]-1,2,4- oxadiazol-5-yl}- 1,2-dihydro- pyridin-2-one | 457 | 458 | 6, Step 1 |
| 266 | 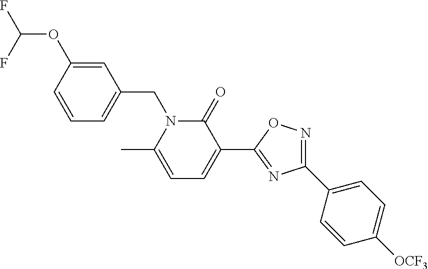 [SMILES: Cc1ccc(-c2nc(-c3ccc(OC(F)(F)F)cc3)no2)c(=O)n1Cc1cccc(OC(F)F)c1] | 1-{[3- (difluoromethoxy) phenyl]methyl}-6- methyl-3-{3-[4- (trifluoromethoxy) phenyl]-1,2,4- oxadiazol-5-yl}- 1,2-dihydro- pyridin-2-one | 493 | 494 | 6, Step 1 |
| 267 | 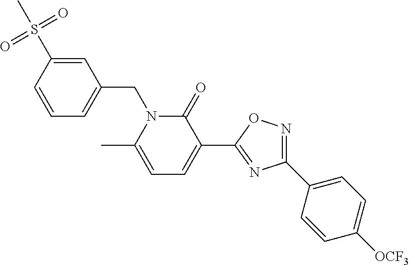 [SMILES: Cc1ccc(-c2nc(-c3ccc(OC(F)(F)F)cc3)no2)c(=O)n1Cc1cccc(S(C)(=O)=O)c1] | 1-[(3- methanesulfonyl- phenyl)methyl]-6- methyl-3-{3-[4- (trifluoromethoxy) phenyl]-1,2,4- oxadiazol-5- yl}-1,2-dihydro- pyridin-2-one | 505 | 506 | 6, Step 1 |
| 268 | 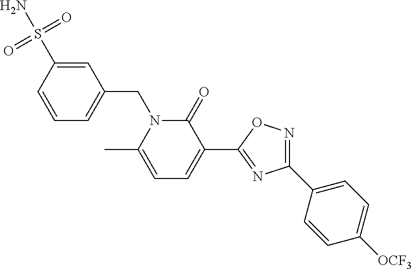 [SMILES: Cc1ccc(-c2nc(-c3ccc(OC(F)(F)F)cc3)no2)c(=O)n1Cc1cccc(S(N)(=O)=O)c1] | 3-[(6-methyl-2- oxo-3-{3-[4- (trifluoromethoxy) phenyl]-1,2,4- oxadiazol-5-yl}- 1,2-dihydro- pyridin-1-yl) methyl]benzene- 1-sulfonamide | 506 | 507 | 6, Step 1 |
| 269 | 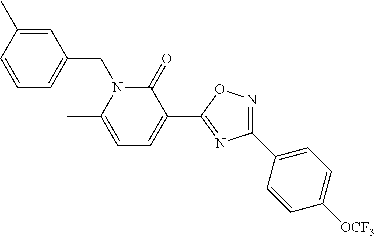 [SMILES: Cc1cccc(Cn2c(C)ccc(-c3nc(-c4ccc(OC(F)(F)F)cc4)no3)c2=O)c1] | 6-methyl-1-[(4- methylphenyl) methyl]-3-{3-[4- (trifluoromethoxy) phenyl]-1,2,4- oxadiazol-5-yl}- 1,2-dihydro- pyridin-2-one | 441 | 442 | 6, Step 1 |
| 270 | 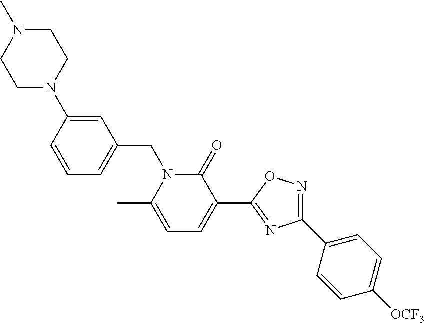 [SMILES: Cc1ccc(-c2nc(-c3ccc(OC(F)(F)F)cc3)no2)c(=O)n1Cc1cccc(N2CCN(C)CC2)c1] | 1-{[3-(4- methylpiperazin- 1-yl)phenyl] methyl}-3-{3-[4- (trifluoromethoxy) phenyl]-1,2,4- oxadiazol-5-yl}- 1,2-dihydro- pyridin-2-one | 511 | 512 | 20, Step 1 |

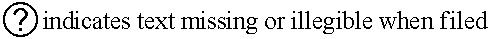

### Cell-Based Reporter Assay for IC₅₀ Determinations

293T-HRE-GFP-luc cells were routinely maintained in DMEM media (high glucose version with GlutaMAX and HEPES, Gibco, catalog #10564) supplemented with 10% fetal bovine serum and 2 μg/mL puromycin (Invitrogen, catalog #A11138-03) using a humidified incubator (normoxia conditions consisting of 37° C., 5% CO₂ and ambient O₂).

In preparation for the reporter assay, cells were harvested and resuspended in DMEM media (high glucose version with GlutaMAX and HEPES) supplemented with 10% fetal bovine serum. Cells were inoculated into 384-well white Culturplates (Perkin Elmer catalog #6007680) at a density of 12,000 cells/well in a volume of 30 L. The microplates were incubated overnight (approximately 17-19 hours) at 37° C. with 5% CO₂ and ambient O₂. Stock solutions of the test compounds were prepared in DMSO (Sigma, Catalog #D2650) and serially diluted 1:3 using DMSO. Compounds were additionally diluted (1:50) with culturemedium and 10 L were added per well to the Culturplate. Following a 30 min. incubation under normoxia conditions, the plates were incubated in hypoxia for 6 hrs. (37° C., 5% CO₂ and 1% O₂). Steadylite Plus (Perkin Elmer, catalog #6016751) was then added (40 L/well), the plates were mixed on an orbital shaker at room temperature in the dark for 15 min., and luminescence was measured using an Envision plate reader (Perkin Elmer). IC₅₀ values were calculated using a four-parameter logistic curve fit. Results are shown below in Table 2; ND indicates no data.

TABLE 2

| | Activity: |
|---|---|
| | A = <100 nM |
| Example | B = 100-1000 nM |
| No. | C = 1-10 uM |
| 1 | A |
| 2 | A |
| 3 | B |
| 4 | A |
| 5 | A |
| 6 | A |
| 7 | A |
| 8 | A |
| 9 | B |
| 10 | A |
| 11 | A |
| 12 | A |
| 13 | A |
| 14 | A |
| 15 | A |
| 16 | A |
| 17 | B |
| 18 | A |
| 19 | A |
| 20 | B |
| 21 | C |
| 22 | B |
| 23 | B |
| 24 | A |
| 25 | A |
| 26 | A |
| 27 | B |
| 28 | B |
| 29 | A |
| 30 | A |
| 31 | A |
| 32 | B |
| 33 | B |
| 34 | A |
| 35 | A |
| 36 | B |
| 37 | A |
| 38 | B |
| 39 | A |
| 40 | A |
| 41 | A |
| 42 | A |
| 43 | A |
| 44 | A |
| 45 | A |
| 46 | A |
| 47 | B |
| 48 | A |
| 49 | A |
| 50 | B |
| 51 | B |
| 52 | A |
| 53 | A |
| 53a | A |
| 53b | A |
| 54 | A |
| 55 | A |
| 55a | A |
| 55b | A |
| 56 | A |
| 57 | A |
| 58 | A |
| 59 | A |
| 60 | A |
| 61 | A |
| 62 | A |
| 63 | A |
| 64 | B |
| 65 | B |
| 66 | B |
| 67 | A |
| 68 | B |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | B |
| 86 | A |
| 87 | A |
| 88 | A |
| 89 | A |
| 90 | A |
| 91 | A |
| 92 | B |
| 93 | A |
| 94 | A |
| 95 | A |
| 96a | A |
| 96b | A |
| 97a | A |
| 97b | A |
| 98 | A |
| 99 | A |
| 100 | A |
| 101 | B |
| 102 | A |
| 103 | A |
| 104 | A |
| 105 | A |
| 106 | A |
| 107 | B |
| 108 | B |
| 109 | B |
| 110 | B |
| 111 | A |
| 112 | B |
| 113 | A |
| 114 | A |
| 115 | A |
| 116 | A |
| 117 | C |
| 118 | A |
| 119 | A |
| 120 | A |
| 121 | A |
| 122 | A |
| 123 | A |
| 124 | A |
| 125 | C |
| 126 | A |
| 127 | B |
| 128 | B |
| 129 | B |
| 130 | B |
| 131 | A |
| 132 | A |
| 133 | A |
| 134 | A |
| 135 | B |
| 136 | A |
| 137 | A |
| 138 | A |
| 139 | A |
| 140 | A |
| 141 | B |
| 142 | B |
| 143 | B |
| 144 | A |
| 145 | A |
| 146 | B |
| 147 | A |
| 148 | A |
| 149 | A |
| 150 | A |
| 151 | B |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | B |
| 171 | A |
| 172 | A |
| 173 | A |
| 174 | B |
| 175 | A |
| 176 | A |
| 177 | A |
| 178 | B |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | B |
| 197 | A |
| 198 | A |
| 199 | B |
| 200 | A |
| 201 | A |
| 202 | B |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | B |
| 216 | B |
| 217 | B |
| 218 | B |
| 219 | B |
| 220 | B |
| 221 | A |
| 222 | B |
| 223 | B |
| 224 | A |
| 225 | B |
| 226 | B |
| 227 | A |
| 228 | B |
| 229 | A |
| 230 | B |
| 231 | B |
| 232 | A |
| 233 | A |
| 234 | B |
| 235 | B |
| 236 | A |
| 237 | B |
| 238 | A |
| 239 | B |
| 240 | A |
| 241 | A |
| 242 | A |
| 243 | A |
| 244 | A |
| 245 | B |
| 246 | A |
| 247 | A |
| 248 | A |
| 249 | A |
| 250 | B |
| 251 | A |
| 252 | A |
| 253 | A |
| 254 | A |
| 255 | A |
| 257 | B |
| 258 | B |
| 259 | A |
| 260 | B |
| 261 | B |
| 262 | A |
| 263 | B |
| 264 | B |
| 265 | B |
| 266 | B |
| 267 | B |
| 268 | B |
| 269 | A |
| 270 | A |

### Diffuse Large B-Cell Lymphoma (DLBCL) Assay

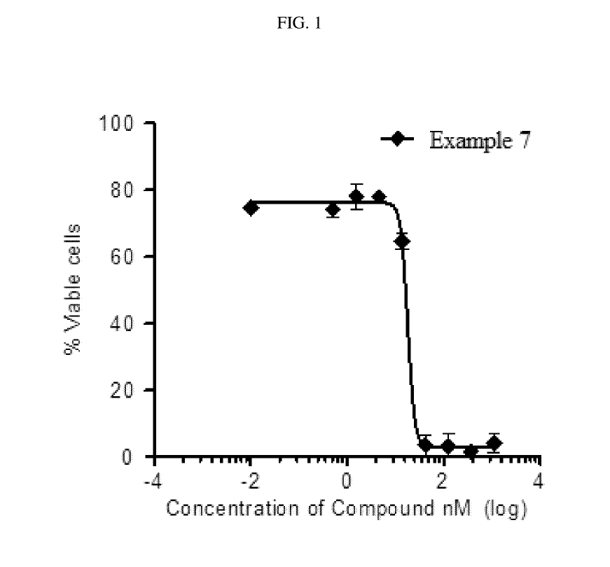

Equal number of TMD8 cells were plated and treated with varying concentrations of the compound of Example 7 for 7 days. Percent of viable cells was determined using Guava ViaCount reagents (EMD Millipore cat #4000-0040) that contains proprietary dyes that enable the determination of the number of live and dead cells in a sample (FIG. 1). TMD8 cells respond robustly to the compound of Example 7 indicating the effectiveness of the compound as an anti-tumor agent in DLBCL.

### Acute Myeloid Leukemia

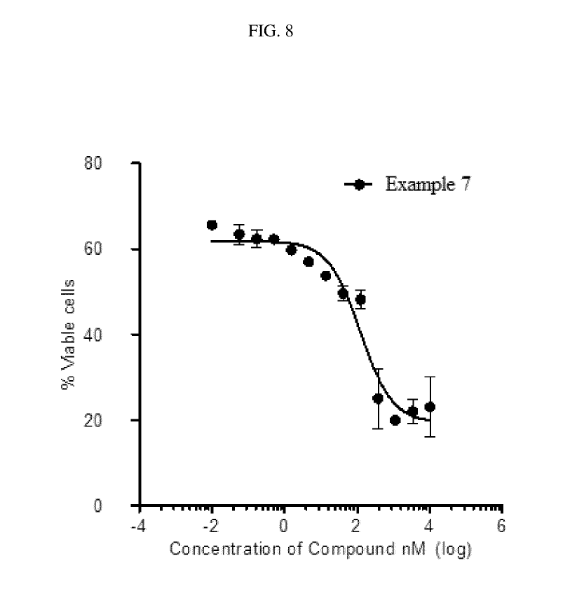

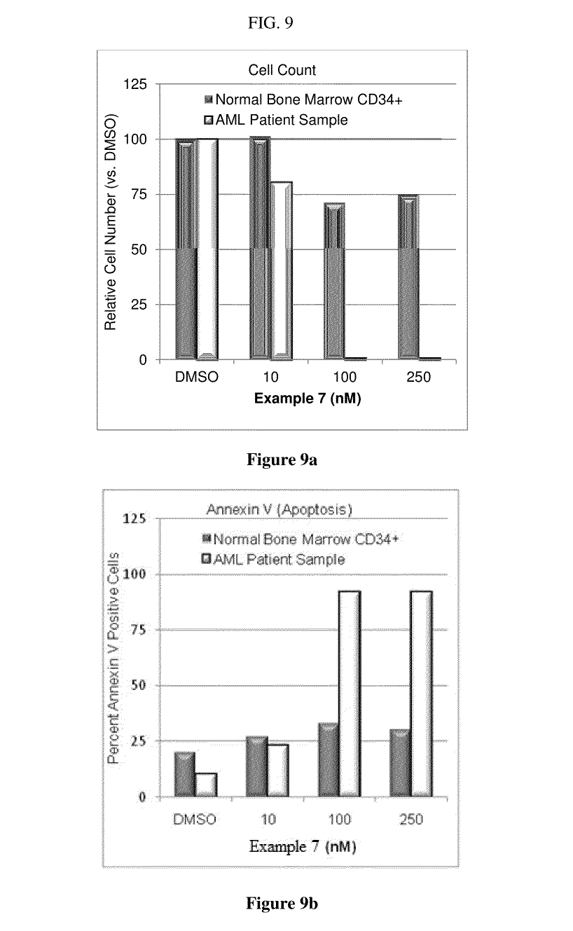

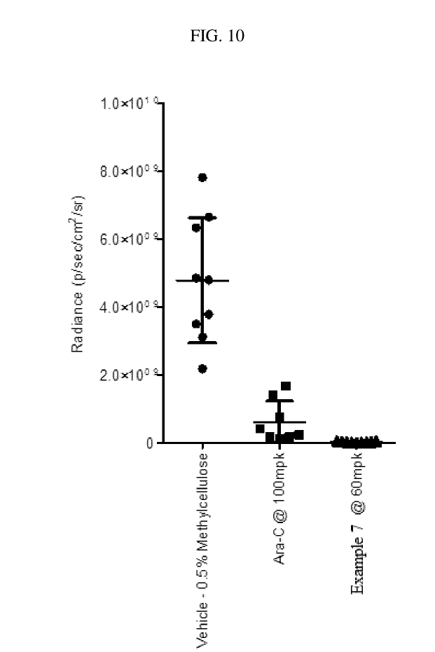

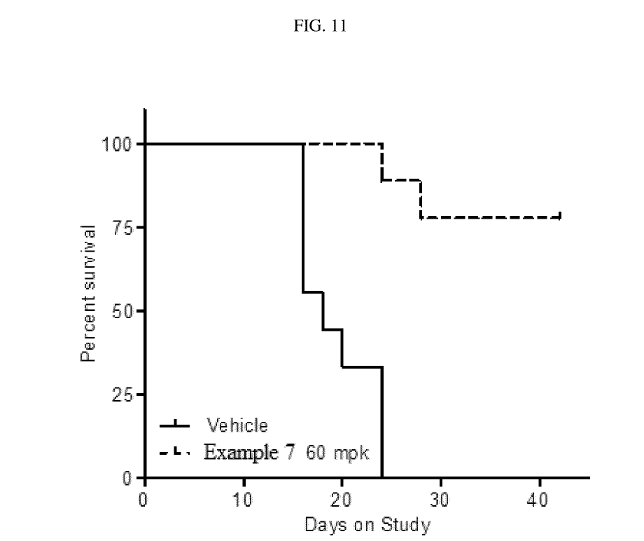

The OCI-AML3 cell line was treated with various concentrations of the compound of Example 7 for 7 days and the percent of viable cells normalized to control cells treated with DMSO (FIG. 8). CD45+ primary AML cells from an AML patient or CD45+ normal bone marrow cells from a healthy volunteer were treated with the compound of Example 7 for 4 days and the number of viable cells and percentage of apoptotic (annexin V) were determined (FIGS. 9a and 9b). A significant decrease in the number of viable cells and significant increase in annexin V cells in the compound of Example 7-treated sample was observed whereas there was a minimal response in normal bone marrow cells. OCI-AML3 cells constitutively expressing luciferase were tail vein injected in NSG nude mice. 17 days after cell injection, luciferin was injected into animals and luciferase signal was measured using an IVIS imaging system to determine tumor burden and for randomization of subjects into study groups. On day 18, animals began receiving daily oral doses of vehicle or 60 mpk of the compound of Example 7 were started and continued throughout the study. On day 28, imaging was performed again to determine tumor burden (FIG. 10). Treatment of tumor cell bearing animals with the compound of Example 7 significantly increased their survival relative to vehicle treated animals (FIG. 11).

### Neuroblastoma and Glioblastoma Cellular Assay and Xenograft Model

Cellular Assay:

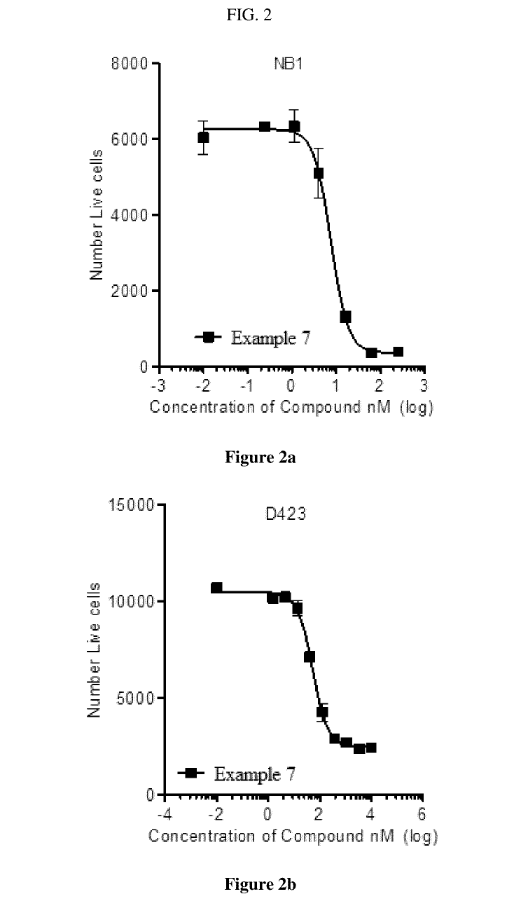

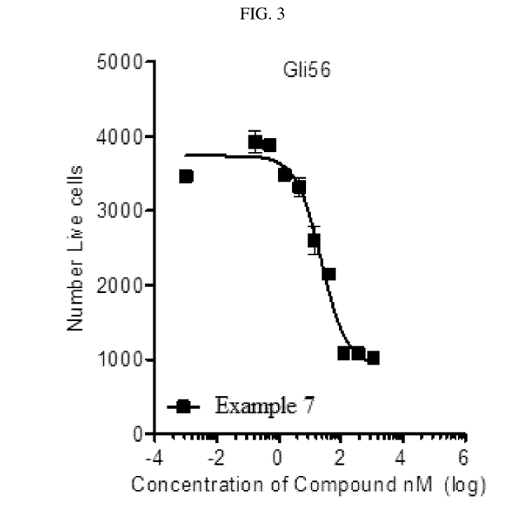

NB-1, Gli56, and D423 cell lines are deleted for ENO-1 (GLI56 and D423) or PGD, which renders them with reduced glycolytic capacity (Muller, F. et al., Nature, 2012, 488, 337-42). When these cell lines are treated with various concentrations of the compound of Example 7, cell number is significantly reduced with cell death readily apparent in NB-1 and Gli56 (FIGS. 2-3).

Xenograft Model:

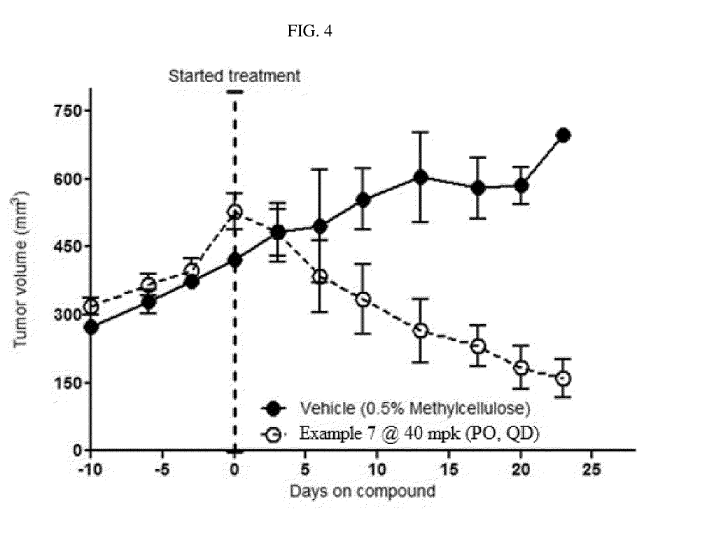

To establish activity and provide in vivo proof of concept, NB-1 cells were implanted into CD-1 nude mice and treated with vehicle or 40 mpk of the compound of Example 7 po daily hen tumors reached 400-500 mm³. Tumor size was measure 3×/week using caliper measurements (FIG. 4).

### In Vivo Murine Xenograft and Models for Tumor Growth Inhibition

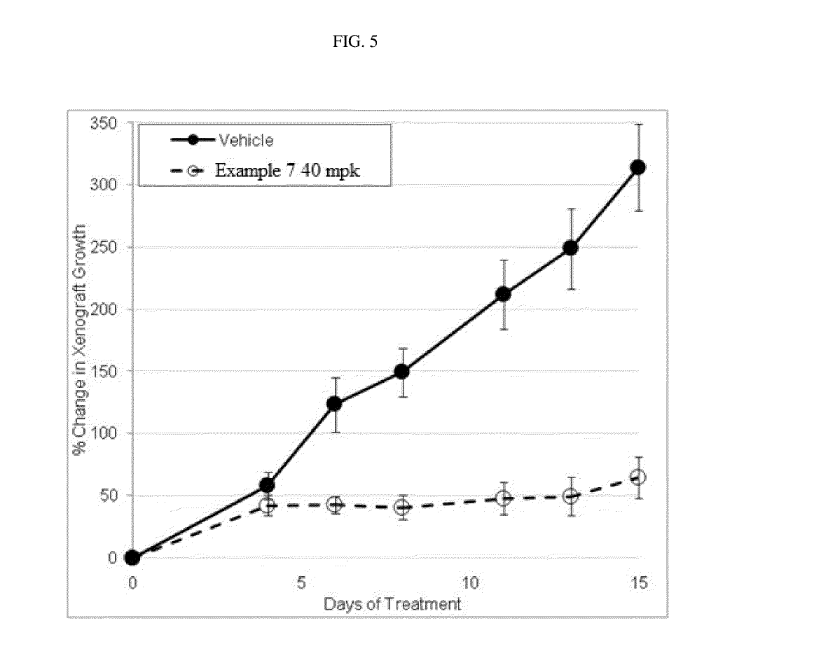

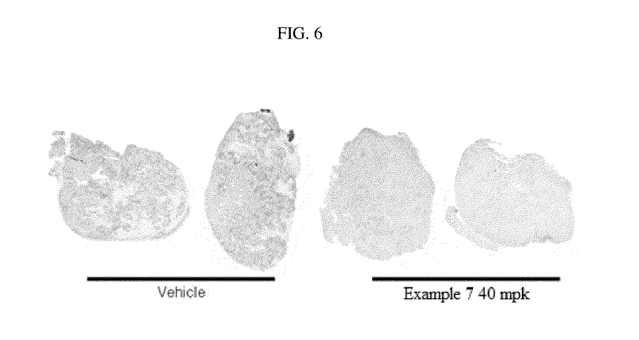

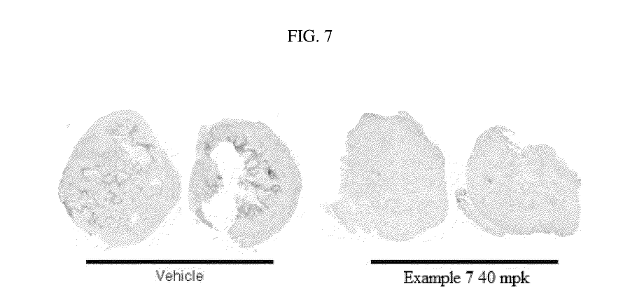

Non-Small Cell Lung Cancer: H460 cells were implanted subcutaneously in CD-1 nude mice and treated with the compound of Example 7 (40 mpk qdx 14) delivered by oral gavage for 14 days. Animals were randomized into study groups and the study initiated when the average tumor volume was 400 mm³. During treatment, tumor volume was measured three times per week to determine tumor growth over the course of the study (FIG. 5). Nine tumor bearing mice were included in each group. On day 15, 3 hours prior to take down, hypoxyprobe (Hypoxyprobe, Inc. cat #HP3) was injected into mice. Tumor sections were stained (dark areas) for the level of hypoxia utilizing an anti-hypoxyprobe antibody and standard IHC methods (FIG. 6). The same tumors were stained for the expression of HIF regulated gene carbonic anhydrase IX (CA9) using standard IHC methods (FIG. 7). Treatment of the mice with the compound of Example 7 inhibited the growth of the H460 xenografts over the course of the study, establishing the anti-tumor activity of the compound. Target engagement, as measure by elimination of hypoxia and CA9 protein expression in the tumor, was achieved establishing that at the anti-tumor activity level, the compound of Example 7 is inhibiting HIF pathway activity.

Head and Neck Cancer:

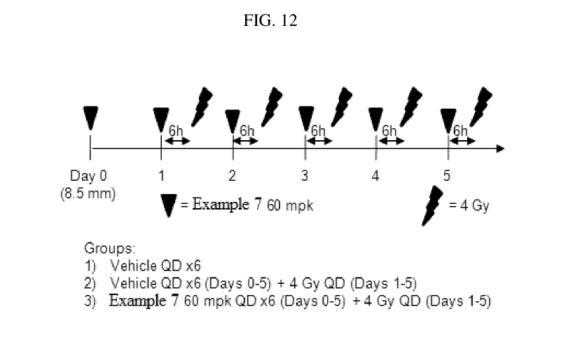

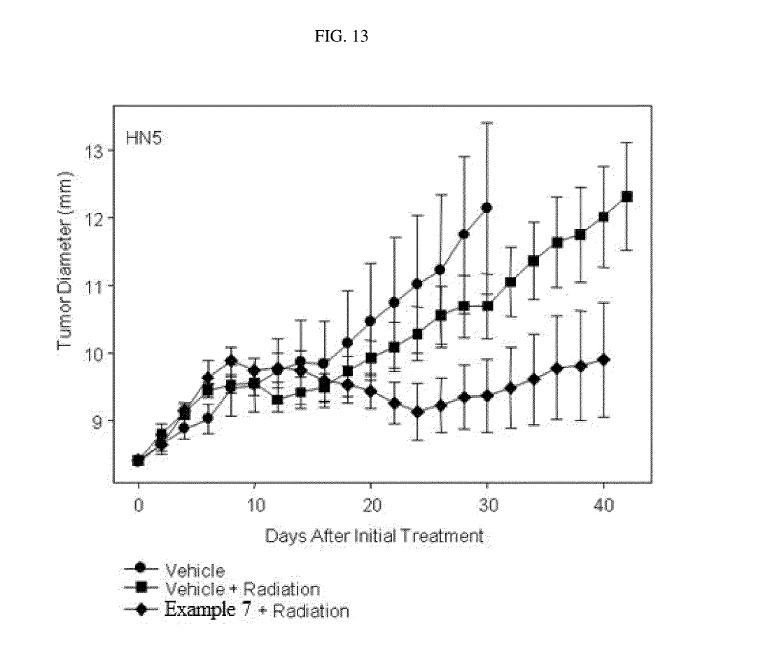

HN5 head and neck cells were injected intramuscularly into CD-1 nude mice. Upon tumors reaching 8.5 mm in diameter, animals were enrolled in the study and received either vehicle or the compound of Example 7 (days 0-5) with or without a 4 Gy dose 6 hours after the compound of Example 7 on days 1-5 of the study (FIG. 12). Tumor size was measured every other day to determine the rate of growth (FIG. 13).

Further examples of xenograft models are given below for glioblastoma cancer.

Glioblastoma Cancer.

In one example of a typical protocol, female athymic nu/nu nude mice, 5 to 6 weeks-old (approx. 18-22 g) may be obtained, for example from Harland Sprague-Dawley, Inc. Nude mice are inoculated with tumor cells. U251, U87-EGFRviii or other human cancer cells, at a concentration of about 1-5×10⁶ in 0.15 ml solution mixed with matrigel and DMEM medium are injected subcutaneously into the right flank of each mouse. When tumor volume reaches around 200 or 600 mm³, animal are randomly assigned to three groups (or more, depending on the umber of dose levels of a compound to be evaluated) and treatment started with test article (for example, at 5 mg/kg/day or 10 mg/kg/day) delivered via oral gavage for up to 21 days. Animals in control group receive the vehicle alone under identical conditions. Tumor volumes are measured by a digital caliper and calculated using the formula (L×W×H)×0.5236. Significant differences are expected to be observed compared with control group (P<0.05, using ANOVA). Animal weight is monitored throughout the experiment. It is expected that no significant difference will be observed between control and treated groups, which further indicates the test article is non-toxic in tumor-bearing nude mice at doses used for inhibiting tumor growth.

The foregoing protocols are versatile, and may be modified to substitute virtually any type of human cancer cell line. Examples include the breast cancer cell lines AG11132A, MCF-7, and T47-D; estrogen, progesterone, and HER-2/neu receptor positive breast cancer cell lines HCC-1428 and ZR-75; estrogen, progesterone, and HER-2/neu receptors negative breast cancer cell lines MDA-231 and BT20; prostate cancer cell lines LNCaP, PC-3, and DU145; colon cancer cell lines DLD-1 and LoVo; ovarian cancer cell lines OVCAR-3 and SK-OV-3; lung cancer cell lines H69AR, NCI-H23, and A549; and pancreatic cancer cell lines Capan-1 and BxPC-3. Additionally, the protocol may be altered to assay the prevention of tumor development by pre-treating with test compound. Combinations of compounds may be tested, and dosing schedules altered to deliver compound in other ways, i.e., by oral gavage, or to skip days of treatment to reduce any toxic signals. Those skilled in the art will recognize and appropriately apply the multitude of variations available.

### REFERENCES

Bardella, C., P. J. Pollard, and I. Tomlinson. 2011. SDH mutations in cancer. Biochim Biophys Acta. 1807:1432-1443.
Ebos, J. M., C. R. Lee, W. Cruz-Munoz, G. A. Bjarnason, J. G. Christensen, and R. S. Kerbel. 2009. Accelerated metastasis after short-term treatment with a potent inhibitor of tumor angiogenesis. Cancer Cell. 15:232-239.
Gill, A. J. 2012. Succinate dehydrogenase (SDH) and mitochondrial driven neoplasia. Pathology. 44:285-292.
Harada, H., M. Inoue, S. Itasaka, K. Hirota, A. Morinibu, K. Shinomiya, L. Zeng, G. Ou, Y. Zhu, M. Yoshimura, W. G. McKenna, R. J. Muschel, and M. Hiraoka. 2012. Cancer cells that survive radiation therapy acquire HIF-1 activity and translocate towards tumour blood vessels. Nat. Commun. 3:783.
Isaacs, J. S., Y. J. Jung, D. R. Mole, S. Lee, C. Torres-Cabala, Y. L. Chung, M. Merino, J. Trepel, B. Zbar, J. Toro, P. J. Ratcliffe, W. M. Linehan, and L. Neckers. 2005. HIF overexpression correlates with biallelic loss of fumarate hydratase in renal cancer: novel role of fumarate in regulation of HIF stability. Cancer Cell. 8:143-153.
Jones, D. T., and A. L. Harris. 2012. Small-molecule inhibitors of the HIF pathway and synthetic lethal interactions. Expert Opin Ther Targets.
Kaelin, W. G., Jr. 2011. Cancer and altered metabolism: potential importance of hypoxia-inducible factor and 2-oxoglutarate-dependent dioxygenases. Cold Spring Harb Symp Quant Biol. 76:335-345.
Kaelin, W. G., Jr., and P. J. Ratcliffe. 2008. Oxygen sensing by metazoans: the central role of the HIF hydroxylase pathway. Mol. Cell. 30:393-402.
Kim, W. Y., and W. G. Kaelin. 2004. Role of VHL gene mutation in human cancer. J Clin Oncol. 22:4991-5004.
Klein, T. J., and P. M. Glazer. 2010. The tumor microenvironment and DNA repair. Semin Radiat Oncol. 20:282-287.
Koi, M., and C. R. Boland. 2011. Tumor hypoxia and genetic alterations in sporadic cancers. J Obstet Gynaecol Res. 37:85-98.
Li, L., X. Lin, M. Staver, A. Shoemaker, D. Semizarov, S. W. Fesik, and Y. Shen. 2005. Evaluating hypoxia-inducible factor-1alpha as a cancer therapeutic target via inducible RNA interference in vivo. Cancer Res. 65:7249-7258.
Maxwell, P. H., M. S. Wiesener, G. W. Chang, S. C. Clifford, E. C. Vaux, M. E. Cockman, C. C. Wykoff, C. W. Pugh, E. R. Maher, and P. J. Ratcliffe. 1999. The tumour suppressor protein VHL targets hypoxia-inducible factors for oxygen-dependent proteolysis. Nature. 399:271-275.
Mazure, N. M., and J. Pouyssegur. 2010. Hypoxia-induced autophagy: cell death or cell survival? Curr Opin Cell Biol. 22:177-180.
Onnis, B., A. Rapisarda, and G. Melillo. 2009. Development of HIF-1 inhibitors for cancer therapy. J Cell Mol. Med. 13:2780-2786.
Paez-Ribes, M., E. Allen, J. Hudock, T. Takeda, H. Okuyama, F. Vinals, M. Inoue, G. Bergers, D. Hanahan, and O. Casanovas. 2009. Antiangiogenic therapy elicits malignant progression of tumors to increased local invasion and distant metastasis. Cancer Cell. 15:220-231.
Pollard, P. J., J. J. Briere, N. A. Alam, J. Barwell, E. Barclay, N. C. Wortham, T. Hunt, M. Mitchell, S. Olpin, S. J. Moat, I. P. Hargreaves, S. J. Heales, Y. L. Chung, J. R. Griffiths, A. Dalgleish, J. A. McGrath, M. J. Gleeson, S. V. Hodgson, R. Poulsom, P. Rustin, and I. P. Tomlinson. 2005. Accumulation of Krebs cycle intermediates and over-expression of HIF1alpha in tumours which result from germline FH and SDH mutations. Hum Mol. Genet. 14:2231-2239.
Poon, E., A. L. Harris, and M. Ashcroft. 2009. Targeting the hypoxia-inducible factor (HIF) pathway in cancer. Expert Rev Mol. Med. 11:e26.
Rohwer, N., and T. Cramer. 2011. Hypoxia-mediated drug resistance: novel insights on the functional interaction of HIFs and cell death pathways. Drug Resist Updat. 14:191-201.
Semenza, G. L. 2012a. Hypoxia-inducible factors in physiology and medicine. Cell. 148:399-408.
Semenza, G. L. 2012b. Hypoxia-inducible factors: mediators of cancer progression and targets for cancer therapy. Trends Pharmacol Sci. 33:207-214.
Wilson, W. R., and M. P. Hay. 2011. Targeting hypoxia in cancer therapy. Nat Rev Cancer. 11:393-410.
Wouters, B. G., and M. Koritzinsky. 2008. Hypoxia signalling through mTOR and the unfolded protein response in cancer. Nat Rev Cancer. 8:851-864.

All references, patents or applications, U.S. or foreign, cited in the application are hereby incorporated by reference as if written herein in their entireties. Where any inconsistencies arise, material literally disclosed herein controls.

What is claimed is:

1. A compound of structural Formula I
(R₁)ₙ-A-Y₁—B-D-E-(R₃)ₚ (I)
or a salt thereof, wherein:
n is 0, 1, or 2;
p is 0, 1, or 2;
q is 0, 1, 2, 3, or 4;
u is 0, 1, or 2;
A is selected from the group consisting of aryl and heteroaryl;
B is selected from the group consisting of 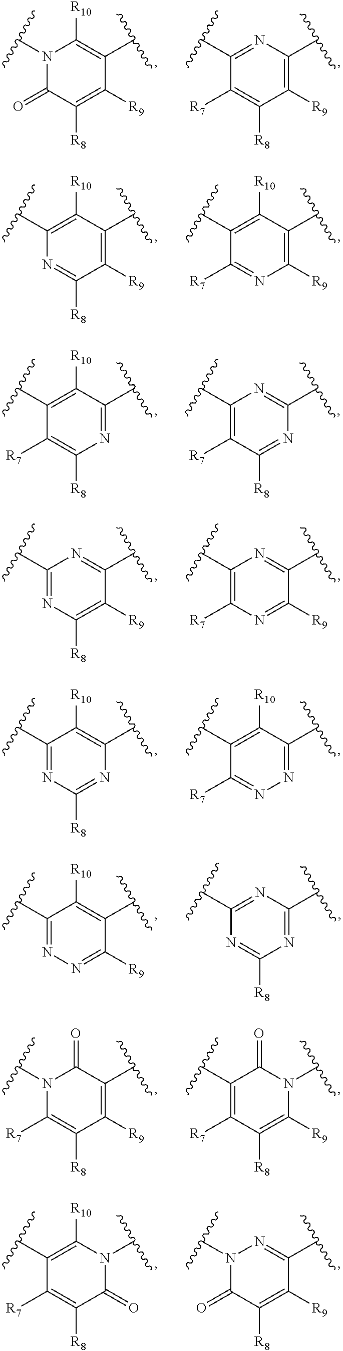 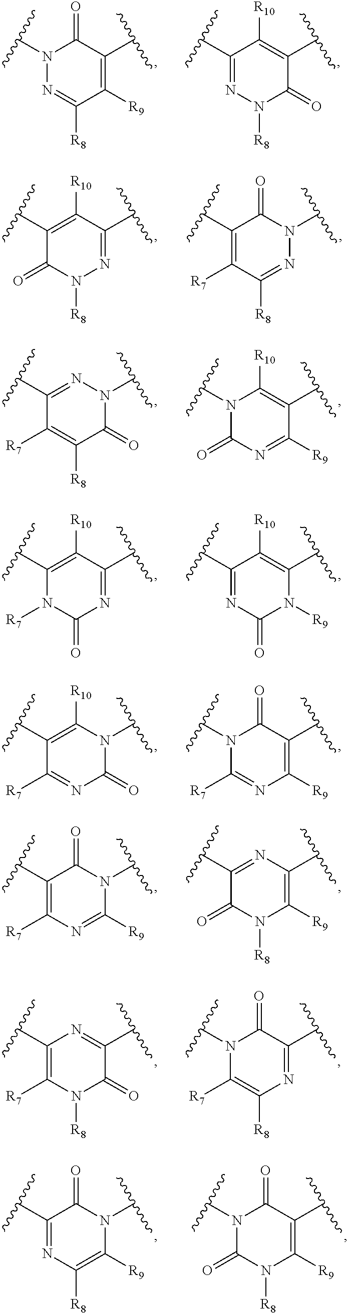 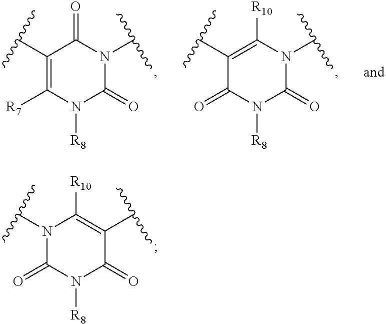
D is selected from the group consisting of alkyl, heteroalkyl, alkoxy, alkylthio, carbonyl, alkylcarbonyl, carboxyl, oxy, thio, sulfinyl, sulfonyl, sulfonamido, amino, amido, alkylamino, and heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, cyano, hydroxy, alkoxy, haloalkoxy, perhaloalkoxy, cycloalkyl, aryl, heterocycloalkyl, heteroaryl, and oxo, any of which may be optionally substituted;
E is selected from the group consisting of aryl and heteroaryl;
G is selected from the group consisting of saturated 3- to 7-membered cycloalkyl and saturated 3- to 7-membered heterocycloalkyl;
R₁ is selected from the group consisting of —Y₂-alkyl-N(R₄)R₅, hydrogen, deuterium, halogen, alkyl, alkenyl, alkynyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, mercaptyl, thiol, sulfonate, sulfonyl, sulfonamido, alkylsulfonyl, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy, 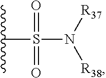
cycloalkylcarbonyl, arylcarbonyl, heterocycloalkylcarbonyl, heteroarylcarbonyl, cycloalkylalkyl, arylalkyl, heterocycloalkylalkyl, heterocycloalkylcarbonylalkyl, and heteroarylalkyl, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, amidoalkyl, acyl, carbonyl, carboxyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, alkoxyalkyl, carboxyl, cyano, hydroxy, alkoxy, haloalkoxy, perhaloalkoxy, oxo, alkylthio, thiol, acylthio, sulfonamido, alkylsulfonyl, amino, amido, carbamate, alkylamino, dialkylamino, alkylaminoalkyl, dialkylaminoalkyl, nitro, trisubstituted silyl, trisubstituted siloxy, cycloalkyl, aryl, heterocycloalkyl, heteroaryl, alkylheterocycloalkyl, any of which may be optionally substituted;
R₃ is selected from the group consisting of hydrogen, deuterium, halogen, alkyl, haloalkyl, perhaloalkyl, heteroalkyl, hydroxyalkyl, alkoxyalkyl, aminoalkyl, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, mercaptyl, thiol, haloalkylthio, perhaloalkylthio, cyanoalkylthio, haloalkylsulfonyl, alkylsulfonyl, alkoxyalkylsulfonyl, cyanoalkylsulfonyl, sulfonate, sulfonamido, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, trisubstituted silyl, —SF₅, —(C(R₃₁)(R₃₂))_{q}—O-alkyl, —(C(R₃₁)(R₃₂))_{q}—O-cycloalkyl, —S(O)ᵤ-alkyl, —S(O)ᵤ-cycloalkyl, cycloalkylthio, —CF₃, —OCF₃, —(C(R₃₁)(R₃₂))_{q}—OCF₃, saturated heterocycloalkyloxy, —(C(R₃₁)(R₃₂))_{q}—O-saturated heterocycloalkyl, —(C(R₃₁)(R₃₂))_{q}-saturated heterocycloalkyl, saturated heterocycloalkylthio, —S(O)ᵤ-saturated heterocycloalkyl, —(C(R₃₁)(R₃₂))_{q}—OCF₃, 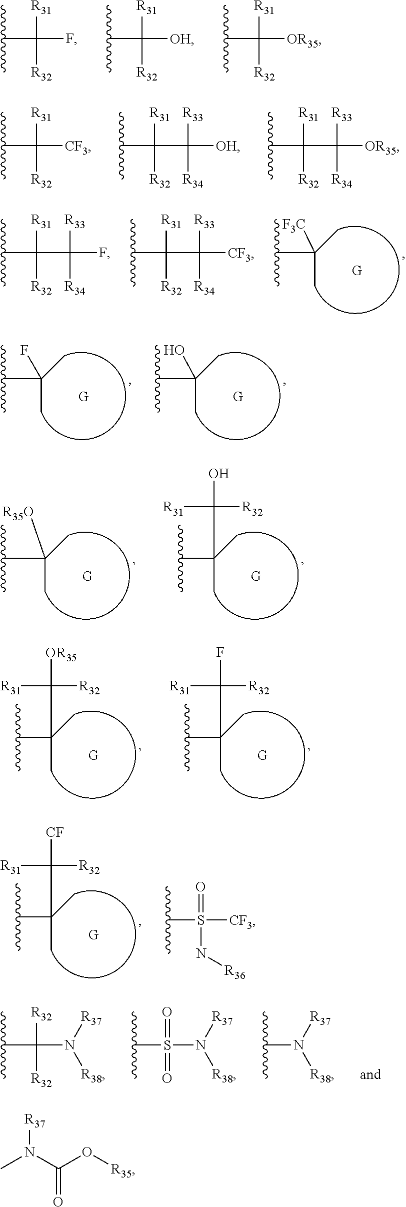
any of which may be optionally substituted;
R₄ and R₅ are independently selected from the group consisting of hydrogen, deuterium, alkyl, alkenyl, alkynyl, haloalkyl, perhaloalkyl, heteroalkyl, hydroxyalkyl, acyl, carbonyl, carboxyl, alkoxy, haloalkoxy, perhaloalkoxy, alkylsulfonyl, sulfonamido, amido, cycloalkyl, aryl, heterocycloalkyl, heteroaryl, cycloalkylalkyl, arylalkyl, heterocycloalkylalkyl, and heteroarylalkyl, or R₄ and R₅, taken together, form a heterocyloalkyl or heteroaryl, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonate, sulfonamido, amino, amido, alkylamino, dialkylamino, carbamate, nitro, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, any of which may be optionally substituted;
R₇, R₈, R₉, and R₁₀ are each independently selected from the group consisting of hydrogen, deuterium, halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, nitro, cycloalkyl, aryl, and heteroaryl, any of which may be optionally substituted;
R₃₁, R₃₂, R₃₃, R₃₄, and R₃₆ are independently selected from the group consisting of hydrogen, deuterium, alkyl, and perfluoroalkyl, any of which can be optionally substituted;
R₃₅ is selected from the group consisting of hydrogen, deuterium, alkyl, perfluoroalkyl, cycloalkyl, and saturated heterocycloalkyl, any of which can be optionally substituted;
R₃₇ and R₃₈ are independently selected from the group consisting of alkyl and perfluoroalkyl, or R₃₇ and R₃₈, taken together, form a heterocyloalkyl, any of which can be optionally substituted;
Y₁ is selected from the group consisting of alkyl, alkenyl, alkynyl, heteroalkyl, alkoxy, alkylthio, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, haloalkyl, perhaloalkyl, heteroalkyl, hydroxyalkyl, aminoalkyl, acyl, carbonyl, carboxyl, cyano, hydroxy, alkoxy, haloalkoxy, perhaloalkoxy, oxo, alkylthio, amino, alkylamino, dialkylamino, and cycloalkyl, any of which may be optionally substituted;
Y₂ is selected from the group consisting of a bond, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, alkenyl, alkynyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonate, sulfonamido, amino, amido, alkylamino, dialkylamino, carbamate, cycloalkyl, aryl, heterocycloalkyl, heteroaryl, cycloalkyloxy, aryloxy, heterocycloalkyloxy, heteroaryloxy, cycloalkylalkyl, arylalkyl, heterocycloalkylalkyl, and heteroarylalkyl, any of which may be optionally substituted;
if A is phenyl, Y₁ is —CH₂—, B is 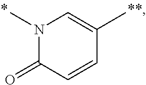
D is 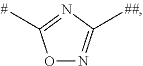
E is phenyl, n is 1, p is 1, and R₃ is —OCF₃, then R₁ is not chloro, bromo, methyl, —NH₂, —NO₂, —C(═O)Cl, —CO₂H, 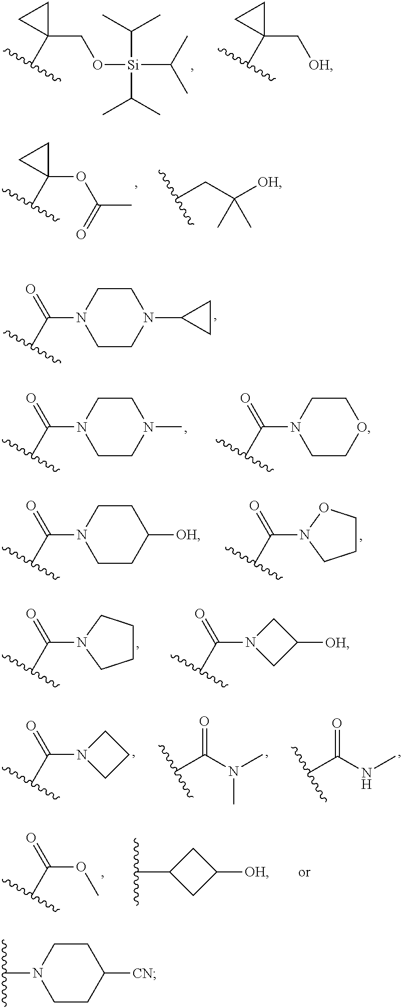
if A is pyridyl, Y₁ is —CH₂—, B is 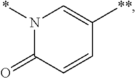
D is 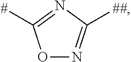
E is phenyl, n is 1, p is 1, and R₃ is —OCF₃, then R₁ is not chloro, bromo, 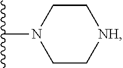 [SMILES: *N1CCNCC1]
—NHCH₃, —NHCH₂CH₃, 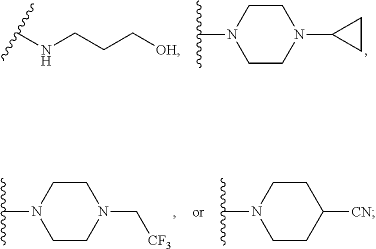
if A is phenyl, Y₁ is —CH₂—, B is 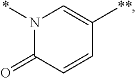
D is 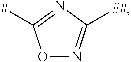
E is phenyl, n is 1, p is 1, and R₃ is —SCF₃, then R₁ is not 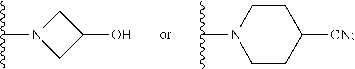 [SMILES: *N1CC(O)C1 ; *N1CCC(C#N)CC1]
if A is pyridyl Y₁ is —CH₂—, B is 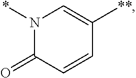
D is 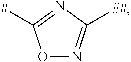
E is phenyl, n is 1, p is 1, and R₃ is —SCF₃, then R₁ is not chloro, 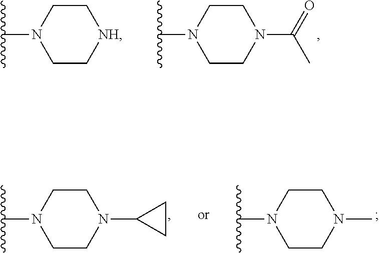
if A is phenyl, Y₁ is —CH₂—, B is 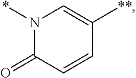
D is 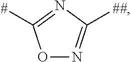
E is phenyl, n is 1, p is 1, and R₃ is —C(CH₃)₂CF₃, then R₁ is not chloro, bromo, methyl, —NH₂, —NO₂, —C(═O)Cl, —CO₂H, 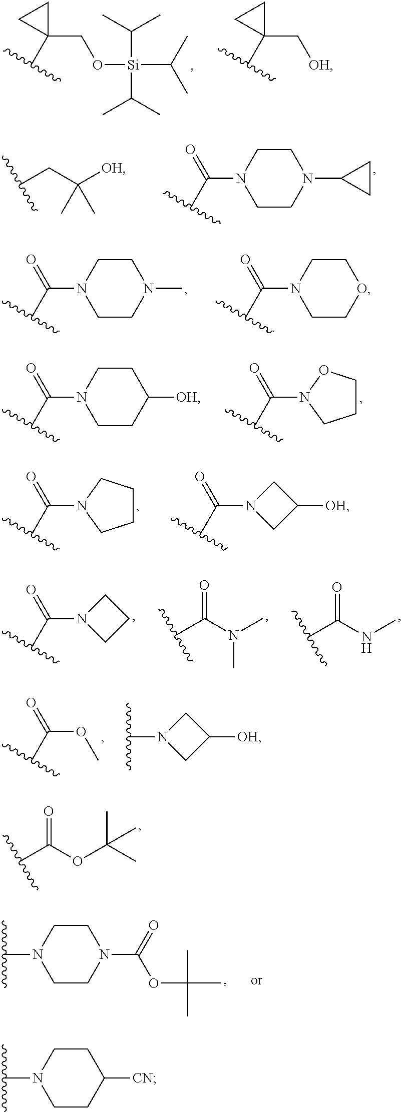
if A is pyridyl, Y₁ is —CH₂—, B is 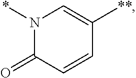
D is 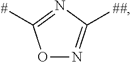
E is phenyl, n is 1, p is 1, and R₃ is —C(CH₃)₂CF₃, then R₁ is not chloro, —NHCH₃, —NHCH₂CH₃, 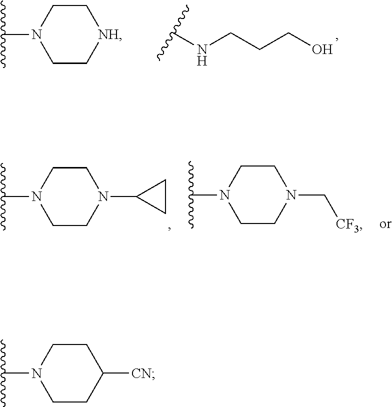
if A is phenyl, Y₁ is —CH₂—, B is 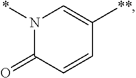
D is 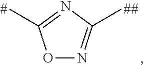
E is phenyl, n is 1, p is 1, and R₃ is 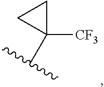 [SMILES: *C1(C(F)(F)F)CC1]
then R₁ is not bromo, 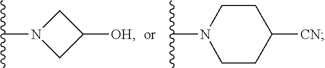 [SMILES: *N1CC(O)C1 ; *N1CCC(C#N)CC1]
if A is pyridyl, Y₁ is —CH₂—, B is 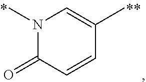
D is 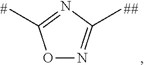
E is phenyl, n is 1, p is 1, and R₃ is 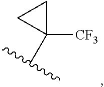 [SMILES: *C1(C(F)(F)F)CC1]
then R₁ is not chloro, 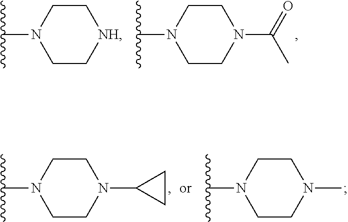
if A is phenyl, Y₁ is —CH₂—, B is 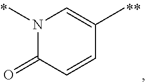
D is 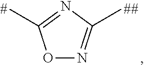
E is phenyl, n is 1, p is 1, and R₃ is methyl, then R₁ is not chloro;
if A is phenyl, Y₁ is —CH₂—, B is 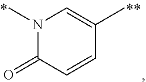
D is 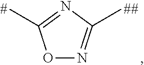
E is phenyl, n is 1, p is 1, and R₃ is chloro, then R₁ is not methyl;
if A is phenyl, Y₁ is —CH₂—, B is 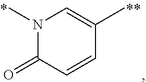
D is 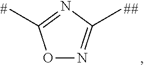
E is phenyl, n is 1, p is 1, and R₃ is methoxy, then R₁ is not methyl;
if A is phenyl, Y₁ is —CH₂—, B is 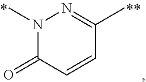
D is 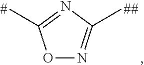
E is phenyl, n is 1, p is 1, and R₃ is —C(CH₃)₂CF₃, then R₁ is not —C(═O)Cl, —CO₂H, 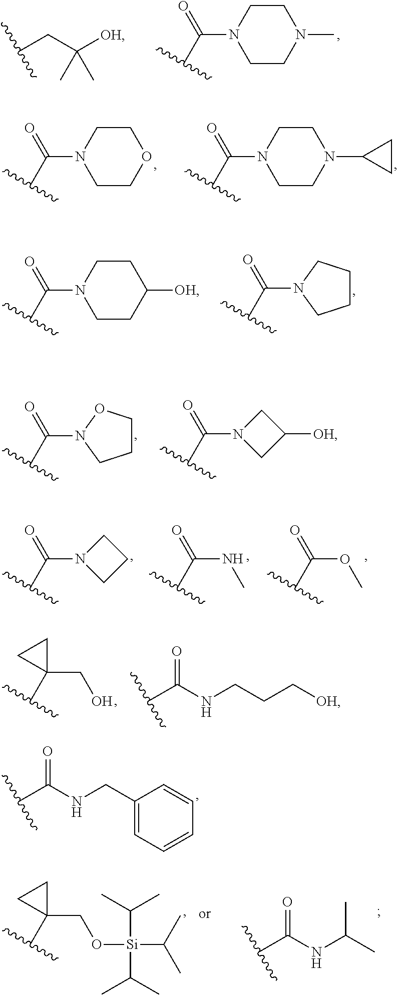
if A is phenyl, Y₁ is —CH₂—, B is 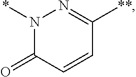
D is 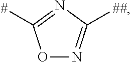
E is phenyl, n is 1, p is 1, and R₃ is —OCF₃, then R₁ is not methyl, —C(═O)Cl, —CO₂H, bromine, 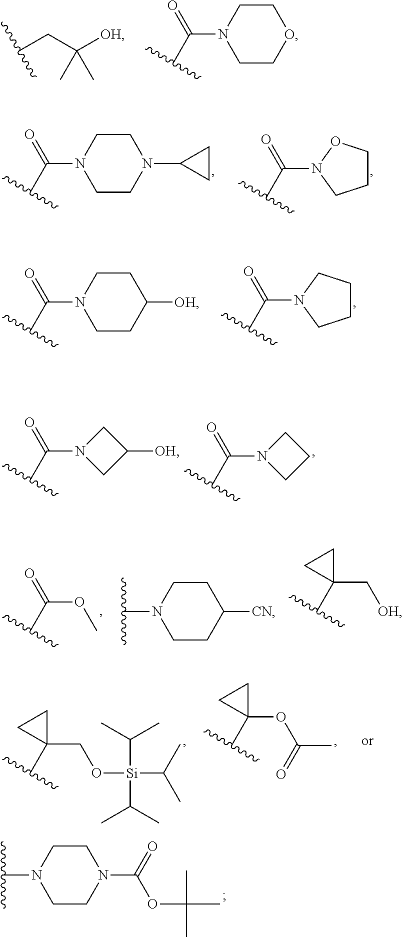
if A is phenyl, Y₁ is —CH₂—, B is 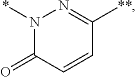
D is 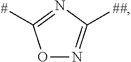
E is phenyl, n is 1, p is 1, and R₃ is methyl, then R₁ is not methoxy;
if A is pyridyl, Y₁ is —CH₂—, B is 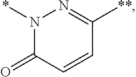
D is 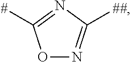
E is phenyl, n is 1, p is 1, and R₃ is —C(CH₃)₂CF₃, then R₁ is not chloro, —NHCH₃, 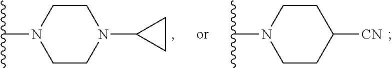 [SMILES: *N1CCN(C2CC2)CC1 ; *N1CCC(C#N)CC1]
if A is pyridyl, Y₁ is —CH₂—, B is 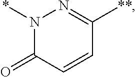
D is 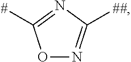
E is phenyl, n is 1, p is 1, and R₃ is —OCF₃, then R₁ is not chloro, —NHCH₃, 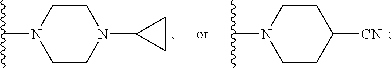 [SMILES: *N1CCN(C2CC2)CC1 ; *N1CCC(C#N)CC1]
and
wherein * represents the point of attachment to Y₁ and ** represents the point of attachment to D, and # represents the point of attachment to B and ## represents the point of attachment to E.

2. The compound as recited in claim 1 wherein:
D is selected from the group consisting of 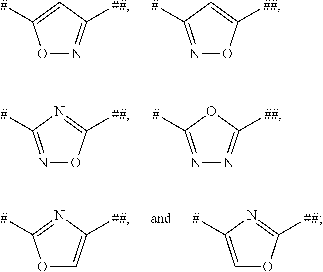
and
# represents the point of attachment to B and ## represents the point of attachment to E.

3. The compound as recited in claim 1 wherein:
if A is 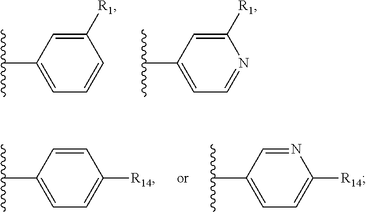
Y₁ is —CH₂—;
B is 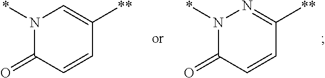
D is 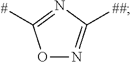
E is 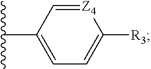
Z₄ is N or CR₁₇;
R₃ is halogen, cyano, —SF₅, tri-C₁-C₄ alkylsilyl, C₁-C₆ alkyl, C₁-C₆ alkoxy, C₁-C₆ alkylthio, C₁-C₆ alkylsulfonyl, C₃-C₆ cycloalkyl, or 4- to 6-membered hererocycloalkyl, wherein said C₁-C₆ alkyl, C₁-C₆ alkoxy, C₁-C₆ alkylthio, and C₁-C₆ alkylsulfonyl are optionally substituted with hydroxy, methoxy, ethoxy, and one to six fluorine atoms, and wherein said C₃-C₆ cycloalkyl and 4- to 6-membered hererocycloalkyl are optionally substituted with one to two substituents selected from the group consisting of fluoro, C₁-C₄ alkyl, trifluoromethyl, hydroxy, methoxy, and ethoxy;
R₁₄ is chloro, cyano, nitro, amino, C₁-C₄ alkyl, C₁-C₄ alkoxy, or C₁-C₄ monoalkylamino, wherein said C₁-C₄ alkyl, C₁-C₄ alkoxy, and C₁-C₄ monoalkylamino are optionally substituted by hydroxyl or one to three fluorine atoms;
R₁₇ is hydrogen, fluoro, chloro, methyl, or trifluoromethyl;
R₂₀₁ and R₂₀₂, taken together with the nitrogen atom to which they are attached, form a 4- to 6-membered heterocycloalkyl which can contain a further heteroatom selected from the group consisting of NR₂₀₅, O, S, and S(O)₂, and which is optionally substituted by one to two substituents selected from the group consisting of fluoro, cyano, C₁-C₄ alkyl, hydroxy, methoxy, and ethoxy, wherein said C₁-C₄ alkyl is optionally substituted with hydroxy and one to three fluorine atoms;
R₂₀₃ and R₂₀₄, is hydrogen or C₁-C₄ alkyl, wherein said C₁-C₄ alkyl is optionally substituted with hydroxy, methoxy, ethoxy, phenyl, and one to three fluorine atoms; or R₂₀₃ and R₂₀₄, taken together with the nitrogen atom to which they are attached, form a 4- to 6-membered heterocycloalkyl which can contain a further heteroatom selected from the group consisting of NR₂₀₅, O, S, and S(O)₂, and which is optionally substituted by one to two substituents selected from the group consisting of fluoro, cyano, C₁-C₄ alkyl, hydroxy, methoxy, and ethoxy, wherein said C₁-C₄ alkyl is optionally substituted with hydroxy and one to three fluorine atoms; and
R₂₀₅ is C₁-C₄ alkyl, C₃-C₆ cycloalkyl, C₁-C₄ alkylcarbonyl, or C₁-C₄ alkoxycarbonyl, wherein said C₁-C₄ alkyl is optionally substituted with one to three fluorine atoms;
then R₁ is not C₁-C₆ alkyl, C₃-C₆ cycloalkyl, C₁-C₄ alkoxycarbonyl, —NR₂₀₁R₂₀₂, or —C(═O)—NR₂₀₃R₂₀₄, wherein said C₁-C₆ alkyl is optionally substituted with hydroxy and one to three fluorine atoms, and said C₃-C₆ cycloalkyl is optionally substituted with a substituent selected from the group consisting hydroxy, C₁-C₄ hydroxyalkyl, and C₁-C₄ alkoxycarbonyl.

4. The compound as recited in claim 1 wherein:
A is selected from the group consisting of aryl and mono- or bicyclic heteroaryl;
B is selected from the group consisting of 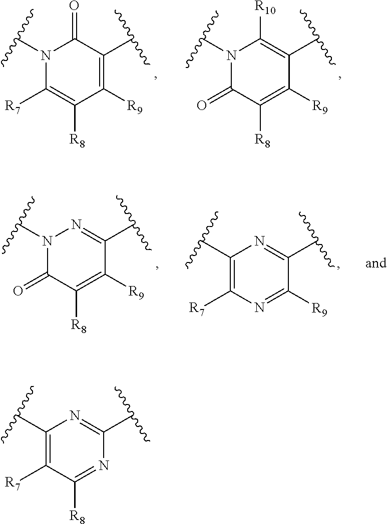
D is selected from the group consisting of amido, 5-membered heteroaryl, and 6-membered heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, cyano, hydroxy, alkoxy, haloalkoxy, perhaloalkoxy, cycloalkyl, aryl, heterocycloalkyl, heteroaryl, and oxo, any of which may be optionally substituted;
E is selected from the group consisting of phenyl, 5-membered heteroaryl, 6-membered heteroaryl, and 9-membered bicyclic heteroaryl;
R₄ and R₅ are independently selected from the group consisting of hydrogen, deuterium, alkyl, alkenyl, alkynyl, haloalkyl, perhaloalkyl, heteroalkyl, hydroxyalkyl, acyl, carbonyl, carboxyl, alkoxy, haloalkoxy, perhaloalkoxy, alkylsulfonyl, sulfonamido, amido, cycloalkyl, aryl, heterocycloalkyl, heteroaryl, cycloalkylalkyl, arylalkyl, heterocycloalkylalkyl, and heteroarylalkyl, or R₄ and R₅, taken together, form a heterocyloalkyl or heteroaryl, any of which can be optionally substituted with one or more substituents selected from the group consisting of hydrogen, deuterium, halogen, alkyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonamido, amino, amido, alkylamino, dialkylamino, carbamate, and cycloalkyl, any of which may be optionally substituted;
R₇, R₈, R₉, and R₁₀ are each independently selected from the group consisting of null, hydrogen, deuterium, halogen, alkyl, haloalkyl, perhaloalkyl, heteroalkyl, hydroxyalkyl, cyano, hydroxy, alkoxy, haloalkoxy, perhaloalkoxy, alkylthio, amino, alkylamino, cycloalkyl, aryl, and heteroaryl;
Y₁ is alkyl, which can be optionally substituted with one or more substituents selected from the group consisting of hydrogen, deuterium, alkyl, cycloalkyl, and halogen; and
Y₂ is selected from the group consisting of a bond, carbonyl, alkylcarbonyl, carboxyl, oxy, thio, sulfinyl, sulfonyl, sulfonamido, amino, amido, alkylamino, and carbamate, any of which can be optionally substituted with one or more substituents selected from the group consisting of hydrogen, deuterium, halogen, alkyl, haloalkyl, perhaloalkyl, heteroalkyl, hydroxyalkyl, acyl, carbonyl, carboxyl, cyano, hydroxy, alkoxy, haloalkoxy, perhaloalkoxy, oxo, alkylthio, mercaptyl, thiol, sulfonamido, amino, amido, alkylamino, dialkylamino, carbamate, and cycloalkyl, any of which may be optionally substituted.

5. The compound as recited in claim 4 wherein:
D is selected from the group consisting of —C(═O)NR₁₁—, 5-membered heteroaryl, and 6-membered heteroaryl;
E is selected from the group consisting of phenyl, pyrimidine, 1,3-benzodioxol, indole, and 1-benzofuran;
R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy, 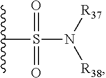
cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;
R₃ is selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted;
R₁₁ is selected from the group consisting of hydrogen, deuterium, alkyl, haloalkyl, perhaloalkyl, heteroalkyl, hydroxyalkyl, cycloalkyl, aryl, heterocycloalkyl, and heteroaryl, any of which may be optionally substituted;
Y₁ is —CH₂—; and
Y₂ is selected from the group consisting of a bond, carbonyl, amino, and alkylamino.

6. The compound as recited in claim 5 wherein:
A is selected from the group consisting of phenyl, 5-membered heteroaryl, and 6-membered heteroaryl;
E is phenyl;
R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, acyl, carboxylalkyl, carboxyl, carbonyl, cyano, hydroxy, alkoxy, haloalkoxy, perhaloalkoxy, oxo, thiolalkyl, sulfonyl, sulfonamido, alkylsulfonyl, amino, amido, alkylamino, dialkylamino, nitro, heterocycloalkyl, heterocycloalkyloxy, 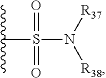
heterocycloalkylcarbonylalkyl, and heterocycloalkylcarbonyl, any of which can be optionally substituted with one or more substituents selected from the group consisting of hydrogen, deuterium, halogen, alkyl, alkenyl, amidoalkyl, acyl, carboxylalkyl, hydroxyalkylcarbonyl, alkynylcarbonyl, heteroalkyl, hydroxyalkyl, alkoxyalkyl, carboxyl, cyano, hydroxy, alkoxy, oxo, sulfonamido, alkylsulfonyl, amino, amido, carbamate, dialkylamino, dialkylaminoalkyl, trisubstituted siloxy, cycloalkyl, heterocycloalkyl, alkylheterocycloalkyl, any of which may be optionally substituted;
R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, and 4- to 6-membered heterocycloalkyl; and
R₁₁ is selected from the group consisting of hydrogen, deuterium, alkyl, and cycloalkyl, any of which may be optionally substituted.

7. The compound as recited in claim 6 wherein:
n is 1;
p is 1; and
R₇, R₈, R₉, and R₁₀ are each independently selected from the group consisting of alkyl, haloalkyl, perhaloalkyl, hydroxy, and cyclopropyl.

8. The compound as recited in claim 5 wherein said compound has structural Formula II 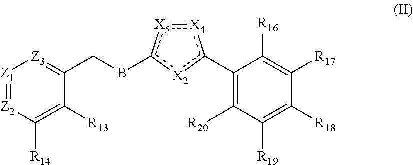
or a salt thereof, wherein:
B is selected from the group consisting of 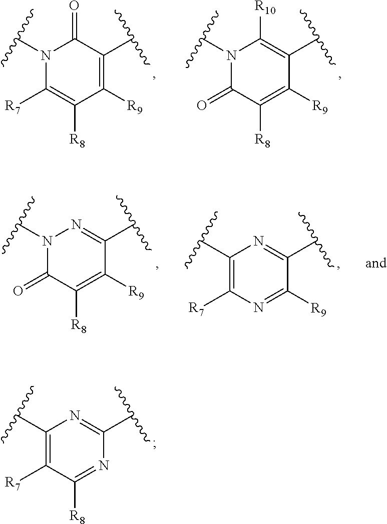
X₂, X₄, and X₅ are independently selected from the group consisting of CR₂₁, N, O, and S, and wherein, X₂, X₄, and X₅, taken together, form a 5-membered heteroaryl;
Z₁ and Z₂ are independently selected from the group consisting of N, NR₁, C═O, and CR₁;
Z₃ is selected from the group consisting of N, NR₁₂, C═O, and CR₁₂;
R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy, 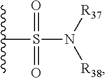
cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;
R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;
R₁₂, R₁₃, and R₁₄ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted;
R₁₆, R₁₉, and R₂₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and cycloalkyl, any of which may be optionally substituted;
R₁₇ and R₁₈ are independently selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted; and
R₂₁ is selected from the group consisting of null, hydrogen, deuterium, halogen, alkyl, haloalkyl, perhaloalkyl, heteroalkyl, hydroxyalkyl, cyano, hydroxy, alkoxy, haloalkoxy, perhaloalkoxy, alkylthio, amino, alkylamino, and dialkylamino.

9. The compound as recited in claim 8 wherein:
two of X₂, X₄, and X₅ are N, and one of X₂, X₄, and X₅ are O; or
one of X₂, X₄, and X₅ is N; one of X₂, X₄, and X₅ is O; and one of X₂, X₄, and X₅ is CH.

10. The compound as recited in claim 8 wherein:
at least one of Z₁ or Z₂ is CR₁;
Z₃ is CR₁₂;
R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, acyl, carboxylalkyl, carboxyl, carbonyl, cyano, hydroxy, alkoxy, haloalkoxy, perhaloalkoxy, oxo, thiolalkyl, sulfonyl, sulfonamido, alkylsulfonyl, amino, amido, alkylamino, dialkylamino, nitro, heterocycloalkyl, heterocycloalkyloxy, 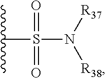
heterocycloalkylcarbonylalkyl, and heterocycloalkylcarbonyl, any of which can be optionally substituted with one or more substituents selected from the group consisting of hydrogen, deuterium, halogen, alkyl, alkenyl, amidoalkyl, acyl, carboxylalkyl, hydroxyalkylcarbonyl, alkynylcarbonyl, heteroalkyl, hydroxyalkyl, alkoxyalkyl, carboxyl, cyano, hydroxy, alkoxy, oxo, sulfonamido, alkylsulfonyl, amino, amido, carbamate, dialkylamino, dialkylaminoalkyl, trisubstituted siloxy, cycloalkyl, heterocycloalkyl, alkylheterocycloalkyl, any of which may be optionally substituted;
R₁₂, R₁₃, and R₁₄ are hydrogen;
R₁₆, R₁₇, R₁₉, and R₂₀ are hydrogen; and
R₂₁ is selected from the group consisting of null, hydrogen, deuterium, halogen, and alkyl.

11. The compound as recited in claim 10 wherein:
R₁ is selected from the group consisting of hydrogen, deuterium, fluorine, bromine, cyano, methyl, isopropyl, 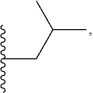 [SMILES: *CC(C)C]
ethylene, 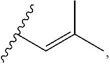 [SMILES: *C=C(C)C]
trifluoromethyl, bromomethyl, hydroxymethyl, difluoromethoxy, methoxy, ethoxy, isopropoxy, hydroxy, nitro, acetyl, carboxyl, —CO₂CH₃, 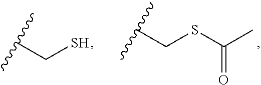
—SO₂CH₃, —SO₂CH₂CH₃, SO₂CH₂CH₂CH₃, —SO₂NH₂, 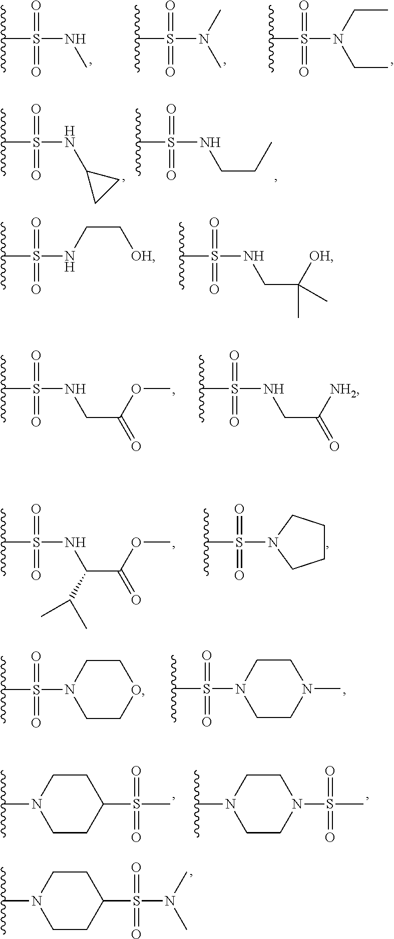
amino, methylamino, dimethylamino, 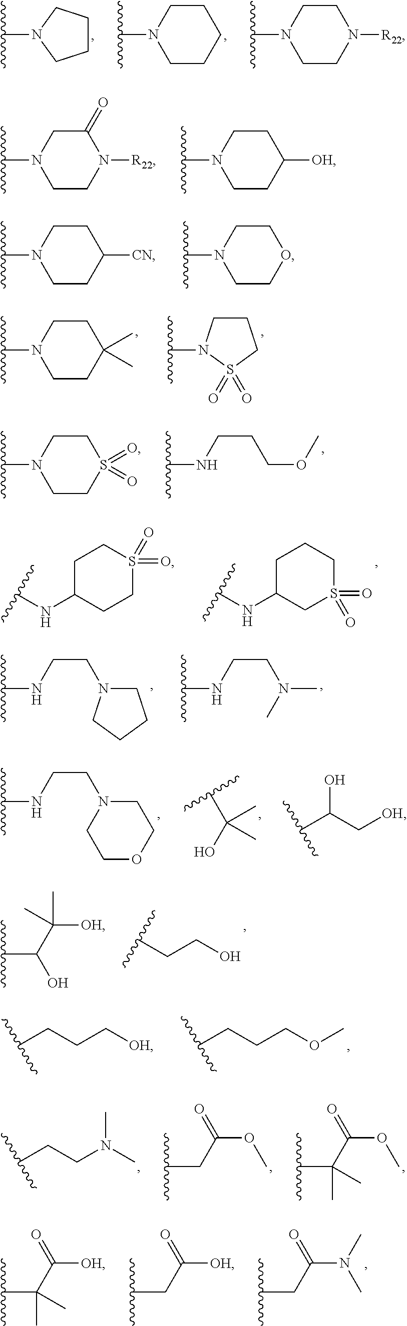 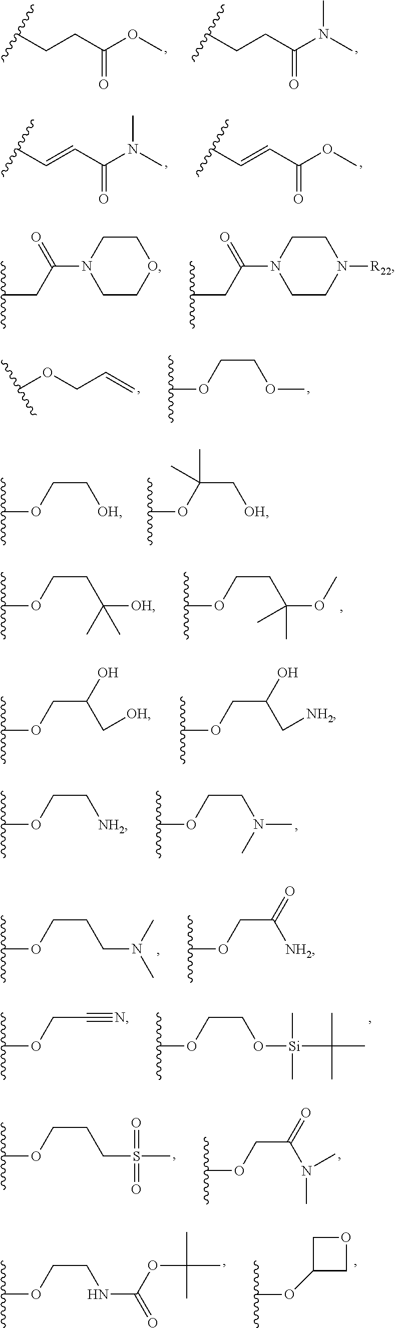 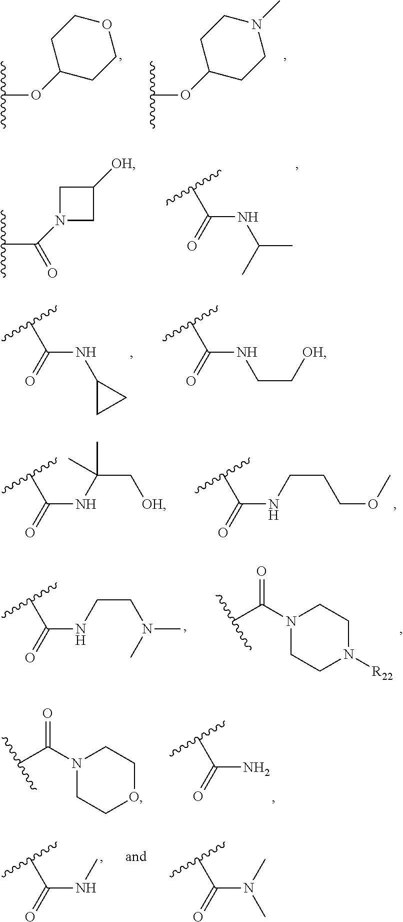
R₇, R₈, R₉, and R₁₀ are each independently selected from the group consisting of hydrogen, deuterium, C₁-C₃ alkyl, 4-pyridyl, and cyclopropyl;
R₁₈ is selected from the group consisting of hydrogen, deuterium, halogen, methyl, isopropyl, tert-butyl, cyclopropyl, cyclohexyl, acetyl, hydroxymethyl, methoxymethyl, methoxy, isopropoxy, methylamino, dimethylamino, methylthio, cyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃, 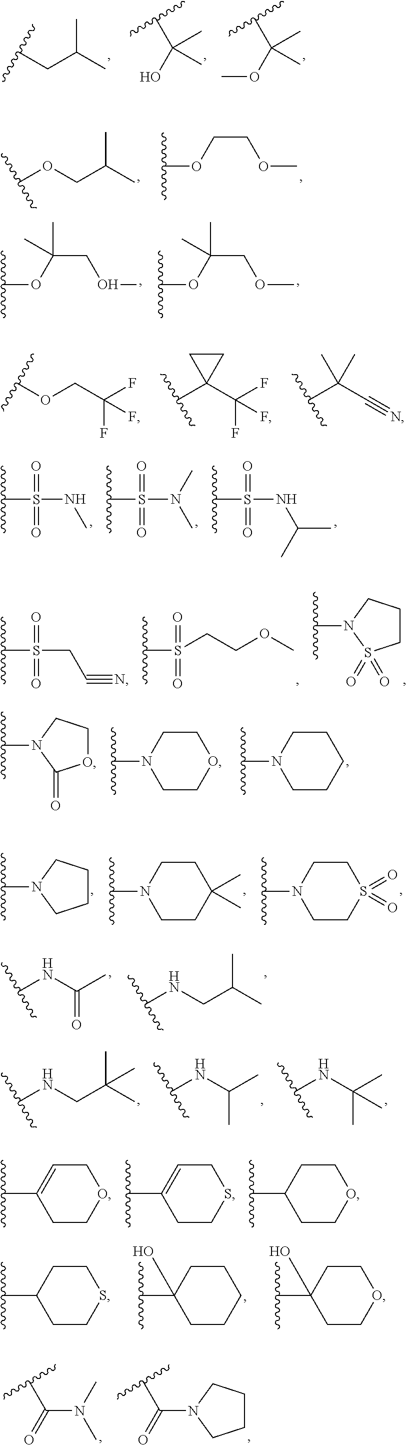
trifluoromethyl, trifluoromethylthio, difluoromethoxy, and trifluoromethoxy; and
R₂₂ is selected from the group consisting of hydrogen, deuterium, methyl, acetyl,

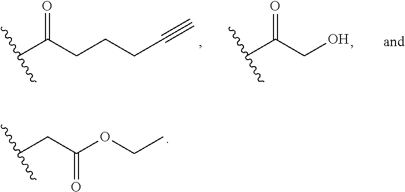

12. The compound as recited in claim 11 wherein:
R₁ is selected from the group consisting of hydrogen, deuterium, chloro, cyano, methyl, ethylene, 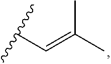 [SMILES: *C=C(C)C]
bromomethyl, hydroxymethyl, difluoromethoxy, methoxy, ethoxy, hydroxy, nitro, —CO₂CH₃, 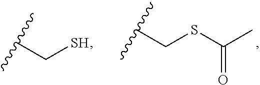
—SO₂CH₃, —SO₂CH₂CH₃, SO₂CH₂CH₂CH₃, —SO₂NH₂, 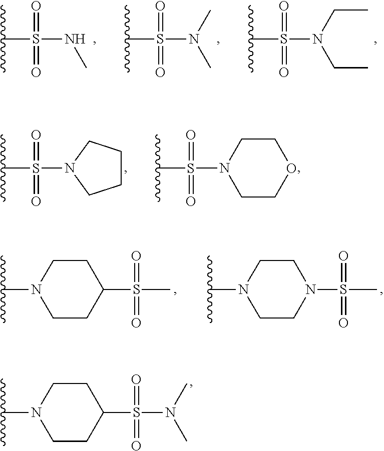
dimethylamino, 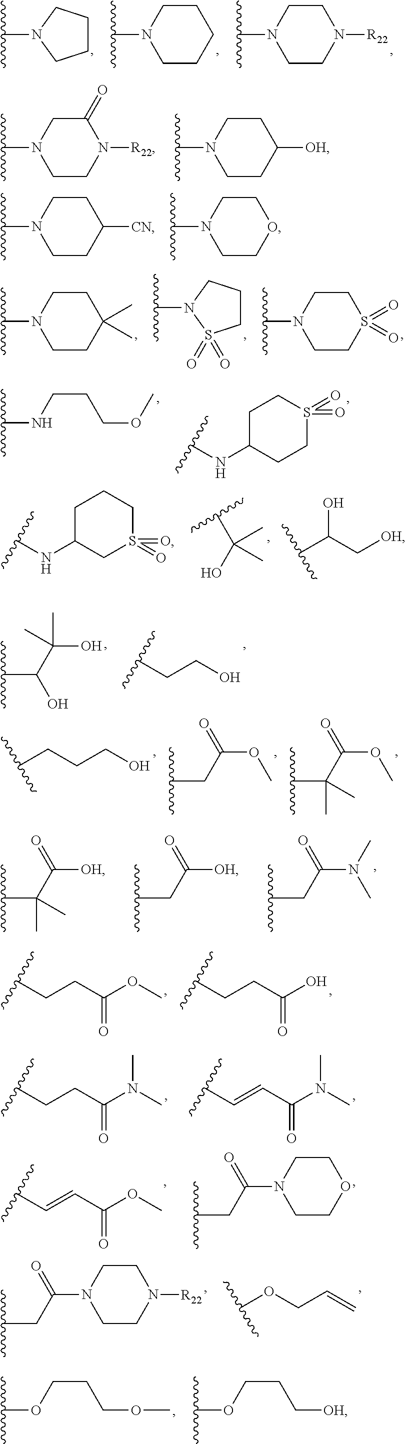 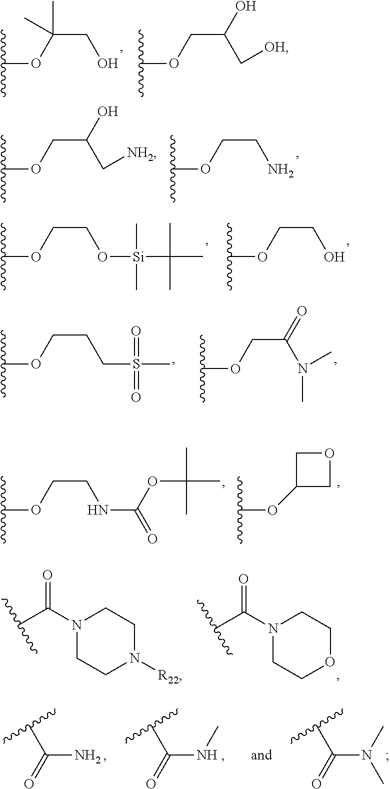
R₇, R₈, R₉, and R₁₀ are each independently selected from the group consisting of hydrogen, deuterium and methyl;
R₁₈ is selected from the group consisting of hydrogen, deuterium, chloro, methyl, isopropyl, tert-butyl, cyclopropyl, cyclohexyl, acetyl, hydroxymethyl, methoxymethyl, methoxy, isopropoxy, methylaminocyanomethyl, cyanomethylthio, cyano, —SO₂CH₃, —SO₂CH(CH₃)₂, —SO₂CH₂CH(CH₃)₂, —SO₂NHCH₂CH₂CH₃, —SO₂CHF₂, —SO₂CF₃, 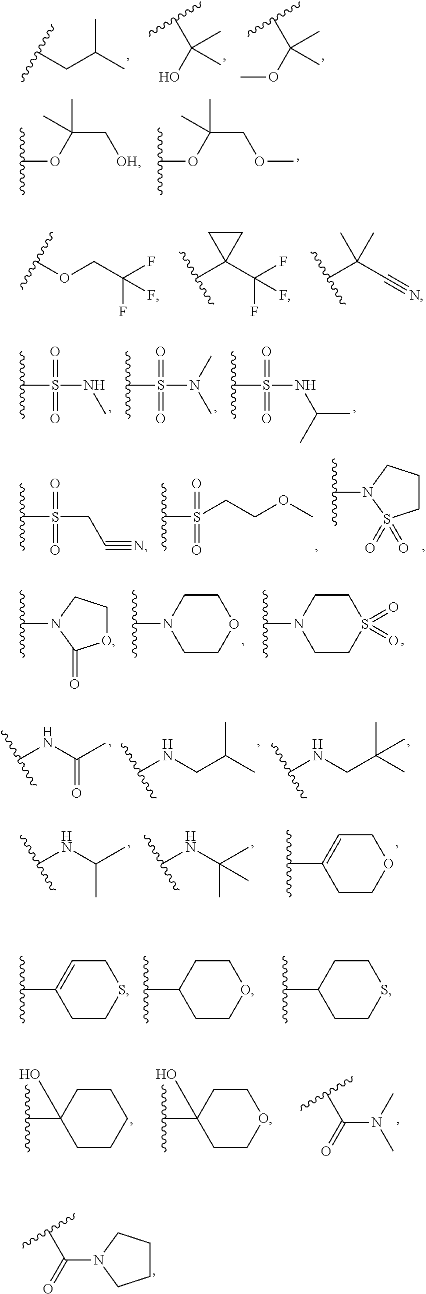
trifluoromethyl, trifluoromethylthio, difluoromethoxy, and trifluoromethoxy; and
R₂₂ is selected from the group consisting of hydrogen, deuterium and methyl.

13. The compound as recited in claim 12 wherein R₇ is selected from the group consisting of hydrogen and C₁-C₃ alkyl.

14. The compound as recited in claim 13 wherein two of X₂, X₄, and X₅ are N, and one of X₂, X₄, and X₅ are O.

15. The compound as recited in claim 13 wherein one of X₂, X₄, and X₅ is N; one of X₂, X₄, is X₅ are O; and one of X₂, X₄, and X₅ is CH.

16. The compound as recited in claim 5 wherein said compound has structural Formula III 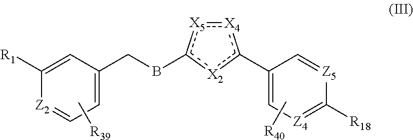
or a salt thereof, wherein:
B is selected from the group consisting of 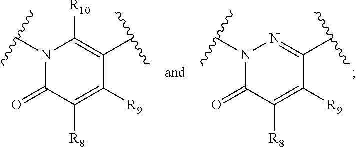
X₄ and X₅ are N and X₂ is O; X₂ and X₅ are N and X₄ is O; X₂ is CH, X₄ is N, and X₅ is O; X₂ is O, X₄ is CH, and X₅ is N; X₂ is CH, X₄ is O, and X₅ is N; X₂ is N, X₄ is CH, and X₅ is O;
Z₂ is selected from the group consisting of N and CR₁₄;
Z₄ is selected from the group consisting of N and CR₁₇;
Zₛ is selected from the group consisting of N and CR₁₉;
R₁ is selected from the group consisting of alkoxy, hydroxyalkyl, dihydroxylkyl, dialkylamidoalkyl, carboxylalkyl, hydroxyalkoxy, dihydroxyalkoxy, alkoxyalkoxy, alkylsulfonylalkoxy, dialkylamidoalkoxy, heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, oxoheterocycloalkyl, dialkylsulfonamido, and alkylsulfonyl, any of which may be optionally substituted with one or more substituents selected from the group consisting hydrogen, hydroxy, alkyl, hydroxyalkylcarbonyl, alkylsulfonyl, alkylsulfonamide, cyano, and oxo;
R₈, R₉, and R₁₀ are each independently selected from the group consisting of hydrogen, deuterium, hydroxyl, alkyl, haloalkyl, perhaloalkyl, cyano, saturated 3-to 6-membered cycloalkyl, 4- to 6-membered heterocycloalkyl, and 5- to 6-membered heteroaryl;
R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and
R₁₈ is selected from the group consisting of hydrogen, alkyl, hydroxyalkyl, hydroxyalkoxy, alkoxyalkyl, alkoxyalkoxy, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

17. The compound as recited in claim 16 wherein:
Z₄ and Z₅ are CH; and
R₃₉ and R₄₀ are hydrogen.

18. The compound as recited in claim 17 wherein R₁ is selected from the group consisting of hydrogen, ethoxy, —SO₂CH₃, —SO₂CH₂CH₃,

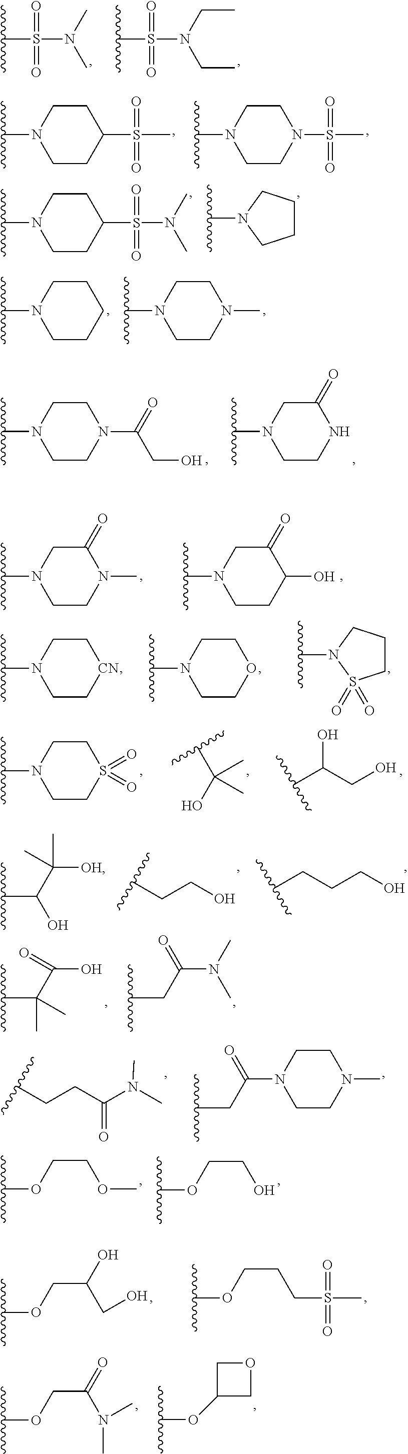

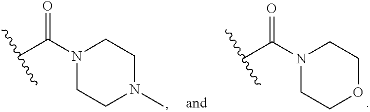

19. The compound as recited in claim 18 wherein R₁ is selected from the group consisting of hydrogen, ethoxy, —SO₂CH₃, —SO₂CH₂CH₃,

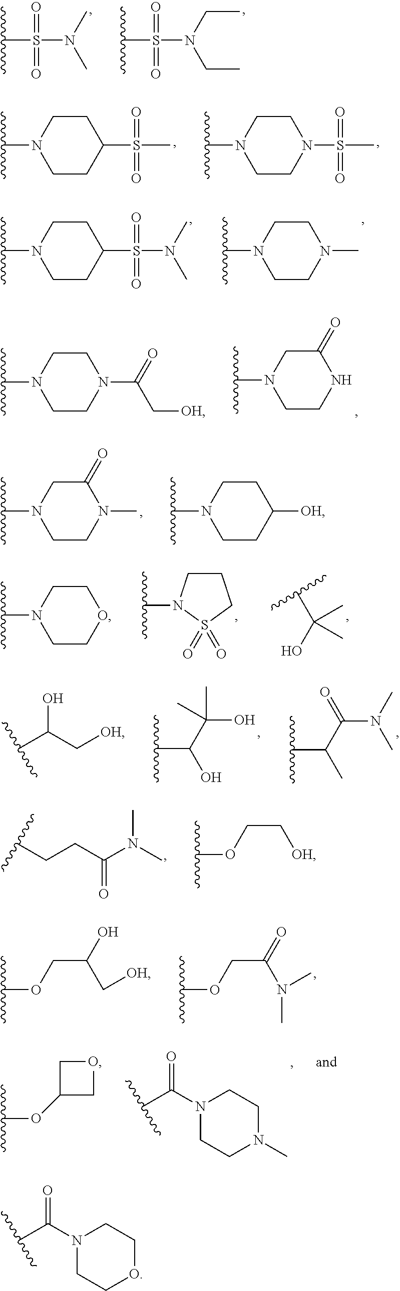

20. The compound as recited in claim 19 wherein R₁ is selected from the group consisting of

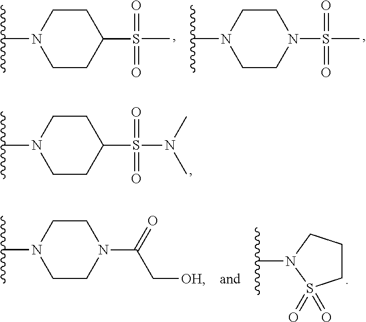

21. The compound as recited in claim 17 wherein R₁₈ is selected from the group consisting of hydrogen, isopropyl, tert-butyl, cyclopropyl, cyclohexyl, hydroxymethyl, isopropoxy, —SF₅, —SCF₃, —SO₂CH₃, —SO₂CHF₂, —SO₂CF₃, 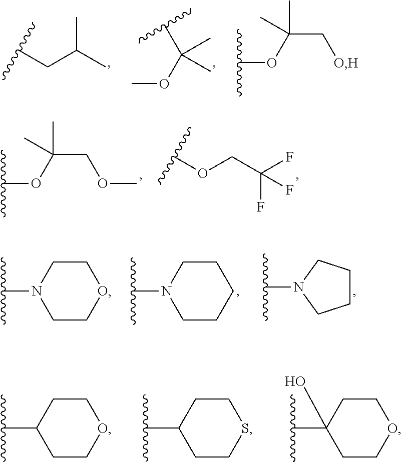
trifluoromethyl, difluoromethoxy, and trifluoromethoxy.

22. The compound as recited in claim 21 wherein R₁₈ is selected from the group consisting of hydrogen, isopropyl, tert-butyl, cyclopropyl, cyclohexyl, isopropoxy, —SO₂CH₃, —SO₂CHF₂, —SO₂CF₃, 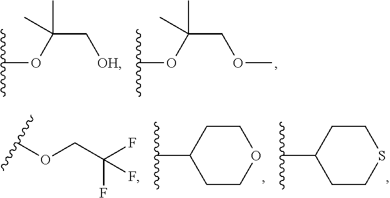
trifluoromethyl, difluoromethoxy, and trifluoromethoxy.

23. The compound as recited in claim 22 wherein R₁₈ is selected from the group consisting of isopropyl, tert-butyl, cyclopropyl, isopropoxy, —SO₂CH₃, —SO₂CF₃, 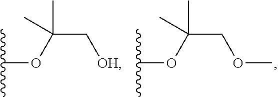
trifluoromethyl, difluoromethoxy, and trifluoromethoxy.

24. The compound as recited in claim 2 wherein said compound has structural Formula IV 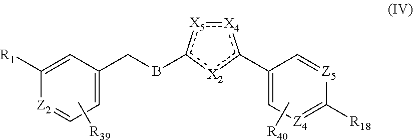
or a salt thereof, wherein:
B is selected from the group consisting of 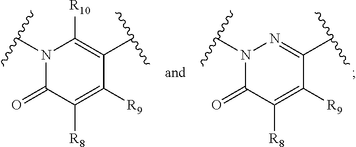
X₂ and X₄ are N and X₅ is O; X₄ and X₅ are N and X₂ is O; X₂ and X₅ are N and X₄ is O; X₂ is CH, X₄ is N, and X₅ is O; X₂ is CH, X₄ is O, and X₅ is N; X₂ is N, X₄ is CH, and X₅ is O;
Z₂ is selected from the group consisting of N and CR₁₄;
Z₄ is selected from the group consisting of N and CR₁₇;
Zₛ is selected from the group consisting of N and CR₁₉;
R₁ is selected from the group consisting of alkoxy, dialkylamidoalkyl, carboxylalkyl, hydroxyalkoxy, alkoxyalkoxy, alkylsulfonylalkoxy, dialkylamidoalkoxy, alkylsulfonylheterocycloalkyl, alkylsulfonamidoheterocycloalkyl, hydroxyalkylcarbonylheterocycloalkyl, oxoheterocycloalkyl, heterocycloalkylalkyl, heterocycloalkyloxy, dialkylsulfonamido, and alkylsulfonyl, any of which may be optionally substituted with one or more substituents selected from the group consisting hydrogen, hydroxy, alkyl, hydroxyalkylcarbonyl, alkylsulfonyl, alkylsulfonamide, cyano, and oxo;
R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;
R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and
R₁₈ is selected from the group consisting of alkyl, hydroxyalkyl, alkoxyalkyl, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

25. The compound as recited in claim 24 wherein:
Z₄ and Z₅ are CH; and
R₁₃ and R₁₆ are hydrogen.

26. The compound as recited in claim 25 wherein R₁ is selected from the group consisting of hydrogen, ethoxy, —SO₂CH₃, —SO₂CH₂CH₃,

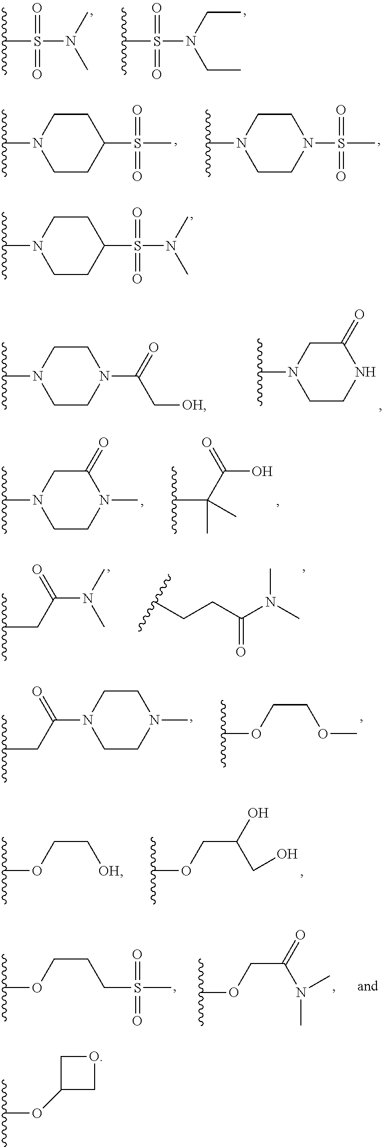

27. The compound as recited in claim 26 wherein R₁ is selected from the group consisting of hydrogen, ethoxy, —SO₂CH₃, —SO₂CH₂CH₃,

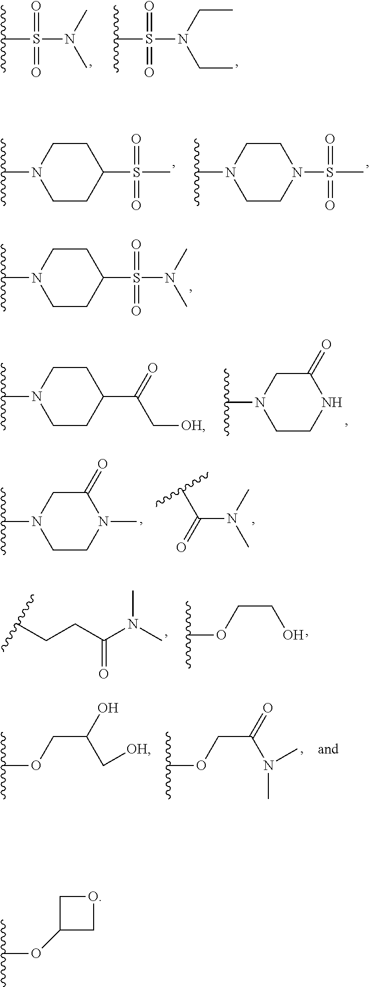

28. The compound as recited in claim 27 wherein R₁ is selected from the group consisting of

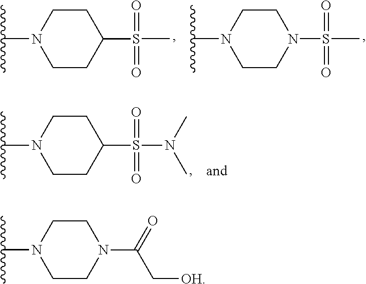

29. The compound as recited in claim 25 wherein R₁₈ is selected from the group consisting of hydrogen, isopropyl, tert-butyl, cyclopropyl, cyclohexyl, hydroxymethyl, isopropoxy, —SO₂CH₃, —SO₂CHF₂, —SO₂CF₃, 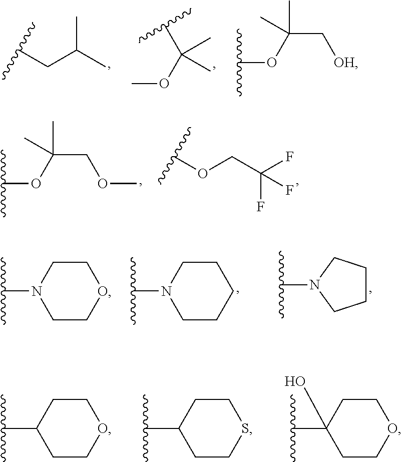
trifluoromethyl, difluoromethoxy, and trifluoromethoxy.

30. The compound as recited in claim 36 wherein R₁₈ is selected from the group consisting of hydrogen, isopropyl, tert-butyl, cyclopropyl, cyclohexyl, isopropoxy, —SO₂CH₃, —SO₂CHF₂, —SO₂CF₃, 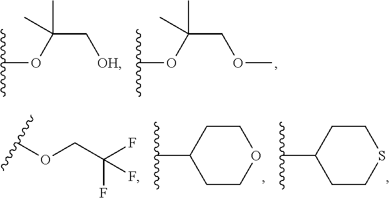
trifluoromethyl, difluoromethoxy, and trifluoromethoxy.

31. The compound as recited in claim 30 wherein R₁₈ is selected from the group consisting of isopropyl, tert-butyl, cyclopropyl, isopropoxy, —SO₂CH₃, —SO₂CF₃, 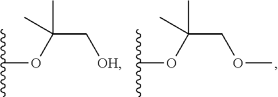
trifluoromethyl, difluoromethoxy, and trifluoromethoxy.

32. The compound as recited in claim 5 wherein said compound has structural Formula V 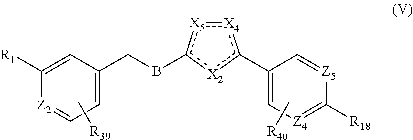
or a salt thereof, wherein:
B is selected from the group consisting of 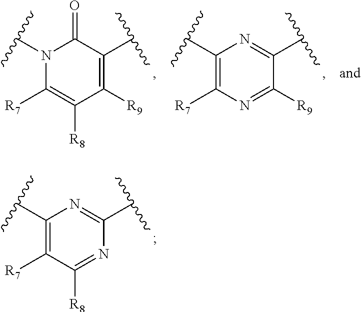
X₂ and X₄ are N and X₅ is O; X₄ and X₅ are N and X₂ is O; X₂ and X₅ are N and X₄ is O; X₂ is CH, X₄ is N, and X₅ is O; X₂ is CH, X₄ is O, and X₅ is N; X₂ is N, X₄ is CH, and X₅ is O;
Z₂ is selected from the group consisting of N and CR₁₄;
Z₄ is selected from the group consisting of N and CR₁₇;
Zₛ is selected from the group consisting of N and CR₁₉;
R₁ is selected from the group consisting of alkoxy, hydroxyalkyl, dihydroxylkyl, dialkylamidoalkyl, carboxylalkyl, hydroxyalkoxy, dihydroxyalkoxy, alkoxyalkoxy, alkylsulfonylalkoxy, dialkylamidoalkoxy, heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, oxoheterocycloalkyl, dialkylsulfonamido, and alkylsulfonyl, wherein said heterocycloalkyl, heterocycloalkylalkyl, heterocycloalkyloxy, heterocycloalkylcarbonyl, alkylsulfonylheterocycloalkyl, alkylsulfonamidoheterocycloalkyl, hydroxyalkylcarbonylheterocycloalkyl, and oxoheterocycloalkyl can be optionally substituted with one or more substituents selected from the group consisting hydrogen, hydroxy, alkyl, hydroxyalkylcarbonyl, alkylsulfonyl, alkylsulfonamide, cyano, and oxo;
R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;
R₁₄, R₁₇, R₁₉, R₃₉, and R₄₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted; and
R₁₈ is selected from the group consisting of hydrogen, alkyl, hydroxyalkyl, hydroxyalkoxy, alkoxyalkyl, alkoxyalkoxy, haloalkyl, perhaloalkyl, alkoxy, haloalkoxy, perhaloalkoxy, alkylthio, haloalkylthio, perhaloalkylthio, alkylsulfonyl, haloalkylsulfonyl, perhaloalkylsulfonyl, cycloalkyl, heterocycloalkyl, hydroxyheterocycloalkyl.

33. The compound as recited in claim 32 wherein:
Z₄ and Z₅ are CH; and
R₁ is selected from the group consisting of hydrogen, ethoxy, —SO₂CH₃, —SO₂CH₂CH₃, 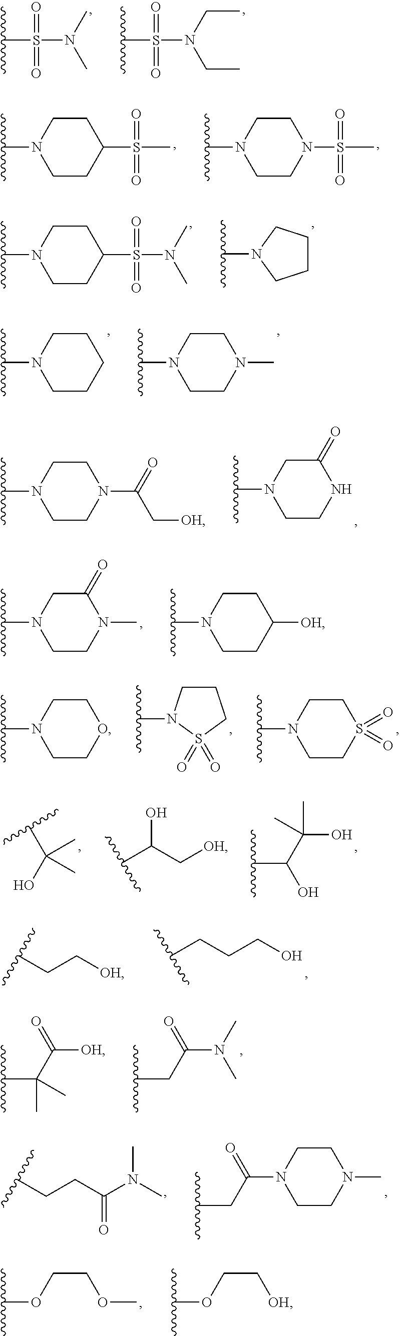 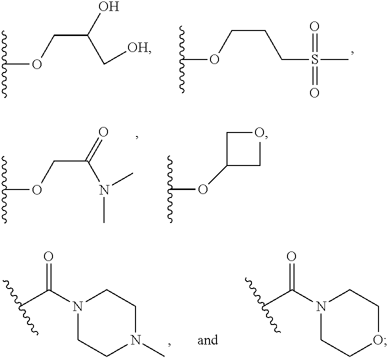
R₁₈ is selected from the group consisting of cyclopropyl, isopropoxy, and 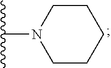 [SMILES: *N1CCCCC1]
and
R₃₉ and R₄₀ are hydrogen.

34. The compound as recited in claim 5 wherein said compound has structural Formula VI 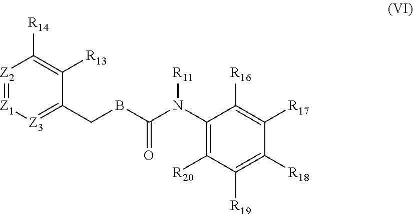
or a salt thereof, wherein:
B is selected from the group consisting of 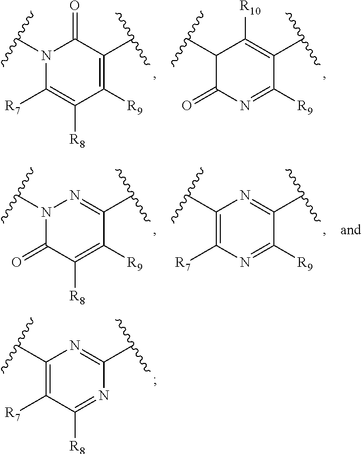
Z₁ and Z₂ are independently selected from the group consisting of N, NR₁, C═O, and CR₁;
Z₃ is selected from the group consisting of N, NR₁₂, C═O, and CR₁₂;
R₁ is selected from the group consisting of —Y₂-alkyl-N(R₄)R₅, hydrogen, deuterium, halogen, alkyl, alkenyl, haloalkyl, perhaloalkyl, heteroalkyl, hydroxyalkyl, aminoalkyl, acyl, carboxylalkyl, carbonyl, carboxyl, carbonyl, cyano, hydroxy, alkoxy, haloalkoxy, perhaloalkoxy, oxo, alkylthio, thiolalkyl, sulfonyl, sulfonamido, alkylsulfonyl, amino, amido, alkylamino, dialkylamino, nitro, cycloalkyl, aryl, heterocycloalkyl, heteroaryl, cycloalkyloxy, aryloxy, heterocycloalkyloxy, heteroaryloxy, 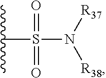
cycloalkylcarbonyl, arylcarbonyl, heterocycloalkylcarbonyl, and heterocycloalkylcarbonylalkyl, any of which can be optionally substituted with one or more substituents selected from the group consisting of hydrogen, deuterium, halogen, alkyl, alkenyl, alkynyl, amidoalkyl, acyl, carboxylalkyl, alkylcarbonyl, heteroalkylcarbonyl, hydroxyalkylcarbonyl, aminoalkylcarbonyl, alkylaminoalkylcarbonyl, alkenylcarbonyl, alkynylcarbonyl, haloalkyl, perhaloalkyl, heteroalkyl, hydroxyalkyl, alkoxyalkyl, carboxyl, cyano, hydroxy, alkoxy, haloalkoxy, perhaloalkoxy, oxo, thiol, acylthio, sulfonamido, alkylsulfonyl, amino, amido, carbamate, alkylamino, dialkylamino, alkylaminoalkyl, dialkylaminoalkyl, trisubstituted silyl, trisubstituted siloxy, cycloalkyl, aryl, heterocycloalkyl, heteroaryl, alkylheterocycloalkyl, any of which may be optionally substituted;
R₇, R₈, R₉, and R₁₀ are each independently selected from the group consisting of hydrogen, deuterium, hydroxyl, alkyl, haloalkyl, perhaloalkyl, cyano, saturated 3- to 6-membered cycloalkyl, 4- to 6-membered heterocycloalkyl, and 5- to 6-membered heteroaryl;
R₁₁ is selected from the group consisting of hydrogen, deuterium, alkyl, haloalkyl, perhaloalkyl, heteroalkyl, hydroxyalkyl, cycloalkyl, aryl, heterocycloalkyl, and heteroaryl;
R₁₂, R₁₃, and R₁₄ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and saturated 3- to 7-membered cycloalkyl, any of which may be optionally substituted;
R₁₆, R₁₉, and R₂₀ are independently selected from the group consisting of hydrogen, deuterium, halogen, alkyl, haloalkyl, perhaloalkyl, cyano, hydroxy, alkoxy, haloalkoxy, perhaloalkoxy, alkylthio, amino, and cycloalkyl, any of which may be optionally substituted; and
R₁₇ and R₁₈ are independently selected from the group consisting of hydrogen, deuterium, halogen, alkyl, haloalkyl, perhaloalkyl, heteroalkyl, hydroxyalkyl, alkoxyalkyl, aminoalkyl, dialkylamino, acyl, carbonyl, carboxyl, cyano, cyanoalkyl, hydroxy, alkoxy, haloalkoxy, perhaloalkoxy, alkoxyalkoxy, hydroxyalkoxy, oxo, alkylthio, haloalkylthio, perhaloalkylthio, cyanoalkylthio, alkylsulfonyl, alkoxyalkylsulfonyl, cyanoalkylsulfonyl, haloalkylsulfonyl, sulfonamido, alkylsulfonamido, amino, alkylamino, dialkylamino, amido, cycloalkyl, aryl, heterocycloalkyl, heteroaryl perhaloalkylcycloalkyl, hydroxyheterocycloalkyl, hydroxycycloalkyl, heterocycloalkylcarbonyl, and heterocycloalkylalkyl, any of which can be optionally substituted.

35. A compound selected from the group consisting of Examples 1 to 23, 25, 27 to 106, 108, 111 to 113, 116 to 132, 135 to 152, and 154 to 270, or a salt thereof.

36. A pharmaceutical composition comprising a compound as recited in claim 1 together with a pharmaceutically acceptable carrier.

37. A method of treatment of a HIF pathway-mediated disease comprising the administration of a therapeutically effective amount of a compound as recited in claim 1 to a patient in need thereof.

38. The method as recited in claim 37 wherein said disease is cancer.

39. The method as recited in claim 38 wherein said cancer is selected from the group consisting of colon cancer, breast cancer, ovarian cancer, lung cancer, prostrate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease, non-Hodgkin's lymphomas, multiple myeloma, hematopoietic malignancies including leukemias (Chronic Lymphocytic Leukemia (CLL), Acute Lymphocytic Leukemia (ALL)) and lymphomas including lymphocytic, granulocytic and monocytic; adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

40. A method of treatment of a disease caused by abnormal cell proliferation comprising the administration of a therapeutically effective amount of a compound as recited in claim 1 to a patient in need thereof.

41. A method of treatment of a HIF pathway-mediated disease comprising the administration of:
a. therapeutically effective amount of a compound as recited in claim 1; and
b. another therapeutic agent.

42. A method for achieving an effect in a patient comprising the administration of a therapeutically effective amount of a compound as recited in claim 1 to a patient, wherein the effect is selected from the group consisting of preventing or reducing resistance to radiotherapy and chemotherapy, preventing or reducing tumor invasion and tumor metastasis, and preventing or reducing angiogenesis.
